SEQUENCE LISTING (132 sequences)

SEQ ID NO: 1 - DNA, 3822 bp, synthetic construct
  misc_feature 1..3822: note = SARS-CoV-2 S Protein
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - protein, 1273 aa, synthetic construct
  REGION 1..1273: note = SARS-CoV-2 S Protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 3822 bp, synthetic construct
  misc_feature 1..3822: note = SARS-CoV-2 D614G S Protein
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 4 - protein, 1273 aa, synthetic construct
  REGION 1..1273: note = SARS-CoV-2 D614G S Protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 672 bp, synthetic construct
  misc_feature 1..672: note = SARS-CoV-2 RBD S Protein
  source 1..672: mol_type = other DNA
cgggtccaacccaccgaaagcattgtgcggttcccaaatatcaccaatctgtgtccctttggcgaagtgttcaatgctacaaggtttgcttctgtgtacgcatggaataggaaacgcatctccaattgtgtcgctgattactccgtgctgtacaattccgcctctttctcaaccttcaagtgttatggcgtttcacctaccaaacttaacgacctgtgcttcactaatgtgtatgccgactcttttgtgatacgaggcgatgaagtgagacagattgcaccagggcagaccggcaaaattgccgactacaactacaagcttccagatgactttaccggatgtgttattgcatggaactcaaacaatctggattccaaggtgggtggcaactataactacctgtatagactgttcaggaaatccaacctgaaaccattcgagcgagatataagcacagaaatctaccaggctggaagtacgccctgcaacggcgtggaagggttcaactgctacttcccattgcagagttacggattccagcctacaaacggggtgggttaccaaccctatcgtgtcgtagtcctgagttttgagctcctccatgccccagccacagtctgtggccccaagaaaagcaccaatctggtgaagaacaaatgcgtgaacttttag

SEQ ID NO: 6 - protein, 224 aa, synthetic construct
  REGION 1..224: note = SARS-CoV-2 RBD S Protein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFG

SEQ ID NO: 7 - DNA, 669 bp, synthetic construct
  misc_feature 1..668: note = SARS-CoV-2 M Protein
  source 1..669: mol_type = other DNA
atggcagattccaacggtactattaccgttgaggaacttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 8 - protein, 222 aa, synthetic construct
  REGION 1..222: note = SARS-CoV-2 M Protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 9 - DNA, 669 bp, synthetic construct
  misc_feature 1..669: note = WA9-UW6 Variant M Protein
  source 1..669: mol_type = other DNA
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctatttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 10 - protein, 222 aa, synthetic construct
  REGION 1..222: note = WA9-UW6 Variant M Protein
MADSNGTITVEELKKLLEQWNLVIGFLFLTWICLLQFAYANRNRFLYIIKLIFLWLLWPVTLACFVLAAIYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 11 - DNA, 669 bp, synthetic construct
  misc_feature 1..669: note = NIHE Variant M Protein
  source 1..669: mol_type = other DNA
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctggtatggccagtaactttagcttgttttgtgcttgctgccgtttacagaataaattggatcaccggtggaattgctatcgcaatggcttgtcttgtacgcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 12 - protein, 222 aa, synthetic construct
  REGION 1..222: note = NIHE Variant M Protein
MADSNGTITVEELKKLLEQWNLVIGFLFLTWICLLQFAYANRNRFLYIIKLIFLWLVWPVTLACFVLAAVYRINWITGGIAIAMACLVRLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 13 - DNA, 669 bp, synthetic construct
  misc_feature 1..669: note = Wuhan_IME_BJO7 Variant M Protein
  source 1..669: mol_type = other DNA
atgtcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 14 - protein, 222 aa, synthetic construct
  REGION 1..222: note = Wuhan_IME_BJO7 Variant M Protein
MSDSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 15 - DNA, 669 bp, synthetic construct
  misc_feature 1..669: note = WA-UW-1753 Variant M Protein
  source 1..669: mol_type = other DNA
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatgtcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 16 - protein, 222 aa, synthetic construct
  REGION 1..222: note = WA-UW-1753 Variant M Protein
MADSNGTITVEELKKLLEQWNLVIGFLFLTWICLLQFAYANRNRFLYIIKLIFLWLLWPVTLACFVLAAVYRINWITGGIAIAMSCLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 17 - DNA, 669 bp, synthetic construct
  misc_feature 1..669: note = WA UW-1755 Variant M Protein
  source 1..669: mol_type = other DNA
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaatgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 18 - protein, 222 aa, synthetic construct
  REGION 1..222: note = WA UW-1755 Variant M Protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRMLSYYKLGASQRVAGDSGFAAYSRYRIGNYKLNTDHSSSSDNIALLVQ

SEQ ID NO: 19 - DNA, 669 bp, synthetic construct
  misc_feature 1..669: note = USA_SC_3572 Variant M Protein
  source 1..669: mol_type = other DNA
atggcaggttccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 20 - protein, 222 aa, synthetic construct
  REGION 1..222: note = USA_SC_3572 Variant M Protein
MAGSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 21 - DNA, 228 bp, synthetic construct
  misc_feature 1..228: note = SARS-CoV-2 E Protein
  source 1..228: mol_type = other DNA
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 22 - protein, 75 aa, synthetic construct
  REGION 1..75: note = SARS-CoV-2 E Protein
MYSFVSEETGTLIVNSVLLFLAFVVFLLVTLAILTALRLCAYCCNIVNVSLVKPSFYVYSRVKNLNSSRVPDLLV

SEQ ID NO: 23 - DNA, 228 bp, synthetic construct
  misc_feature 1..228: note = USA_WA-UW-1588 Variant E Protein
  source 1..228: mol_type = other DNA
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcttgatcttctggtctaa

SEQ ID NO: 24 - protein, 75 aa, synthetic construct
  REGION 1..75: note = USA_WA-UW-1588 Variant E Protein
MYSFVSEETGTLIVNSVLLFLAFVVFLLVTLAILTALRLCAYCCNIVNVSLVKPSFYVYSRVKNLNSSRVLDLLV

SEQ ID NO: 25 - DNA, 228 bp, synthetic construct
  misc_feature 1..228: note = SNU01 Variant E Protein
  source 1..228: mol_type = other DNA
atgtactcattcgtttcggaagagacaggtacgttaatagttaatagcgtacttctttttcttgctttcgtggtattcttgctagttacactagccatccttactgcgcatcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 26 - protein, 75 aa, synthetic construct
  REGION 1..75: note = SNU01 Variant E Protein
MYSFVSEETGTLIVNSVLLFLAFVVFLLVTLAILTAHRLCAYCCNIVNVSLVKPSFYVYSRVKNLNSSRVPDLLV

SEQ ID NO: 27 - DNA, 1260 bp, synthetic construct
  misc_feature 1..1260: note = SARS-CoV-2 N Protein
  source 1..1260: mol_type = other DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 28 - protein, 419 aa, synthetic construct
  REGION 1..419: note = SARS-CoV-2 N Protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 29 - DNA, 234 bp, synthetic construct
  misc_feature 1..234: note = SV40 p(A) tail
  source 1..234: mol_type = other DNA
ctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgg

SEQ ID NO: 30 - DNA, 225 bp, synthetic construct
  misc_feature 1..225: note = bGH p(A) tail
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 31 - DNA, 742 bp, synthetic construct
  misc_feature 1..742: note = Human CMV promoter
  source 1..742: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatc

SEQ ID NO: 32 - DNA, 419 bp, synthetic construct
  misc_feature 1..418: note = SV40 promoter
  source 1..419: mol_type = other DNA
gcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 33 - DNA, 508 bp, synthetic construct
  misc_feature 1..508: note = CAG promoter
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 34 - protein, 14 aa, synthetic construct
  REGION 1..14: note = Chikungunya virus antigen
PPFGAGRPGQFGDI

SEQ ID NO: 35 - protein, 9 aa, synthetic construct
  REGION 1..9: note = Chikungunya virus antigen
TAECKDKNL

SEQ ID NO: 36 - protein, 9 aa, synthetic construct
  REGION 1..9: note = Chikungunya virus antigen
VRYKCNCGG

SEQ ID NO: 37 - DNA, 251 bp, synthetic construct
  misc_feature 1..251: note = SV40 Enhancer
  source 1..251: mol_type = other DNA
ggacctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagt

SEQ ID NO: 38 - DNA, 601 bp, synthetic construct
  misc_feature 1..601: note = hEF1-HTLV Promoter
  source 1..601: mol_type = other DNA
gctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgcagttacagatccaagctgtgaccggcgcctacctgagatcaccggtcaccatggagatcaaggtgctgtttgccctcatctgtattgctgttgctgaggca

SEQ ID NO: 39 - DNA, 2010 bp, synthetic construct
  misc_feature 1..2010: note = SARS-CoV-2 D614G Spike Truncated S1
  source 1..2010: mol_type = other DNA
gttaatctcaccacccgaactcaactcccacccgcatatacaaattccttcaccagaggagtgtactatcctgacaaagtgtttcggtcaagtgtcctccactctactcaggacctctttctgcctttcttttctaacgttacatggtttcatgcaatccatgtgtctgggacaaacggcaccaaacgcttcgacaaccctgtattgccattcaatgatggggtgtactttgcctccacagagaaatccaacatcattcgaggatggattttcgggactactctggactcaaagacacagagcctgctgatcgttaacaacgccacaaacgttgtcatcaaagtgtgcgaattccagttttgcaatgatcccttcctgggagtgtactatcacaagaataacaagtcctggatggagagcgaatttcgggtctacagcagcgcaaacaactgcaccttcgagtacgtgagtcaaccctttctgatggacctggaagggaaacagggaaacttcaagaacctgagagagtttgtctttaagaacatcgacggctattttaagatctatagtaagcatacgcctatcaacctggtaagggatcttccccagggcttttcagccctggaacctttggttgacttgcctattggtatcaatatcaccagatttcagacccttctggcattgcatcggtcttatcttactccaggtgattcctcctccgggtggactgccggcgccgctgcctactatgtcggctatctgcaaccaagaacgttcctgctcaagtacaacgaaaacggcactattacggatgctgttgattgtgccctggaccctctgtctgagactaaatgcaccctcaagagctttaccgttgagaaggggatttaccaaaccagtaatttccgggtccaacccaccgaaagcattgtgcggttcccaaatatcaccaatctgtgtccctttggcgaagtgttcaatgctacaaggtttgcttctgtgtacgcatggaataggaaacgcatctccaattgtgtcgctgattactccgtgctgtacaattccgcctctttctcaaccttcaagtgttatggcgtttcacctaccaaacttaacgacctgtgcttcactaatgtgtatgccgactcttttgtgatacgaggcgatgaagtgagacagattgcaccagggcagaccggcaaaattgccgactacaactacaagcttccagatgactttaccggatgtgttattgcatggaactcaaacaatctggattccaaggtgggtggcaactataactacctgtatagactgttcaggaaatccaacctgaaaccattcgagcgagatataagcacagaaatctaccaggctggaagtacgccctgcaacggcgtggaagggttcaactgctacttcccattgcagagttacggattccagcctacaaacggggtgggttaccaaccctatcgtgtcgtagtcctgagttttgagctcctccatgccccagccacagtctgtggccccaagaaaagcaccaatctggtgaagaacaaatgcgtgaactttaactttaacggactcacaggaaccggcgtattgacggagagtaacaagaagttcctgccattccagcagttcggtcgcgatattgccgacactaccgacgctgtccgagatccccagacattggagattcttgatatcacaccctgtagtttcggcggagtgagcgtgattacgcccggaaccaataccagcaatcaggttgccgtcctgtatcaggacgtgaattgcaccgaggtacctgtcgccatccacgctgaccaacttacacccacatggcgagtatattccaccggctccaacgtctttcagacacgtgctggatgtctgatcggtgcagaacacgttaataatagctacgagtgtgatatccccatcggtgctggaatatgcgcctcttatcaaactcaaaccaactctcctaggcgggcacgt

SEQ ID NO: 40 - protein, 670 aa, synthetic construct
  REGION 1..670: note = SARS-CoV-2 D614G Spike Truncated S1
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 41 - DNA, 568 bp, synthetic construct
  misc_feature 1..568: note = IRES Bridge
  source 1..568: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacga

SEQ ID NO: 42 - DNA, 648 bp, synthetic construct
  misc_feature 1..648: note = Mouse IL-12 p35
  source 1..648: mol_type = other DNA
atgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

SEQ ID NO: 43 - protein, 215 aa, synthetic construct
  REGION 1..215: note = Mouse IL-12 p35
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 44 - DNA, 1008 bp, synthetic construct
  misc_feature 1..1008: note = Mouse IL-12 p40
  source 1..1008: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatagacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag

SEQ ID NO: 45 - protein, 335 aa, synthetic construct
  REGION 1..335: note = Mouse IL-12 p40
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNIDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 46 - DNA, 660 bp, synthetic construct
  misc_feature 1..660: note = Human IL-12 p35
  source 1..660: mol_type = other DNA
atgggtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 47 - protein, 219 aa, synthetic construct
  REGION 1..219: note = Human IL-12 p35
MGPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 48 - DNA, 989 bp, synthetic construct
  misc_feature 1..989: note = Human IL-12 p40
  source 1..989: mol_type = other DNA
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagac

SEQ ID NO: 49 - protein, 328 aa, synthetic construct
  REGION 1..328: note = Human IL-12 p40
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 50 - skipped

SEQ ID NO: 51 - DNA, 59 bp, synthetic construct
  misc_feature 1..59: note = AgeIMhis-F
  source 1..59: mol_type = other DNA
accggcgcctacctgagatcaccggtaacgtagataagcgcttgccacaacccgggatc

SEQ ID NO: 52 - DNA, 72 bp, synthetic construct
  misc_feature 1..72: note = pA-R
  source 1..72: mol_type = other DNA
tatttgaatgtatttagaaaaataaacaaatagggaaatgaagagcccataccacatttgtagaggttttac

SEQ ID NO: 53 - DNA, 57 bp, synthetic construct
  misc_feature 1..57: note = AgeIsp1-F
  source 1..57: mol_type = other DNA
accggcgcctacctgagatcaccggtgccgccaccatggagatcaaggtgctgtttg

SEQ ID NO: 54 - DNA, 80 bp, synthetic construct
  misc_feature 1..80: note = IRESSalI-R
  source 1..80: mol_type = other DNA
tgagggaagcggccgcccgggtcgactctagaggatcccgggttgtggcaagcttatcatcgtgtttttcaaaggaaaac

SEQ ID NO: 55 - skipped

SEQ ID NO: 56 - DNA, 41 bp, synthetic construct
  misc_feature 1..41: note = PspXI-SV40pA-F
  source 1..41: mol_type = other DNA
cggctaagcggccgcagcctcgagcagctagctggccagac

SEQ ID NO: 57 - DNA, 34 bp, synthetic construct
  misc_feature 1..34: note = Cls-SapI-R
  source 1..34: mol_type = other DNA
tatttgaatgtatttagaaaaataaacaaatagg

SEQ ID NO: 58 - DNA, 48 bp, synthetic construct
  misc_feature 1..48: note = Cls-BglII-R-2
  source 1..48: mol_type = other DNA
attggacatgagccaatataaatgtacgaagatctgtcgagccatgtg

SEQ ID NO: 59 - DNA, 25 bp, synthetic construct
  misc_feature 1..25: note = Cls-XmnI-R
  source 1..25: mol_type = other DNA
agaactttaaaagtgctcatcattg

SEQ ID NO: 60 - DNA, 23 bp, synthetic construct
  misc_feature 1..23: note = CLS-PspXI-IRES-F
  source 1..23: mol_type = other DNA
atacgtaagcggccgcagcctcg

SEQ ID NO: 61 - DNA, 24 bp, synthetic construct
  misc_feature 1..24: note = IRES-R
  source 1..24: mol_type = other DNA
tcgtgtttttcaaaggaaaaccac

SEQ ID NO: 62 - DNA, 47 bp, synthetic construct
  misc_feature 1..47: note = Mhis-F
  source 1..47: mol_type = other DNA
acgtggttttcctttgaaaaacacgatgataagcttgccacaacccg

SEQ ID NO: 63 - DNA, 20 bp, synthetic construct
  misc_feature 1..20: note = CLS-M-R
  source 1..20: mol_type = other DNA
atcatgtctggccagctagc

SEQ ID NO: 64 - DNA, 49 bp, synthetic construct
  misc_feature 1..49: note = CLS-XbaI-M-F
  source 1..49: mol_type = other DNA
ctagactcgagacagtccaccacaatctagagccaccatggctgactct

SEQ ID NO: 65 - DNA, 47 bp, synthetic construct
  misc_feature 1..47: note = CLS-MluI-M-R
  source 1..47: mol_type = other DNA
actcatcaatgtatcttatcatgtctgctcgaacgcgtttattgtac

SEQ ID NO: 66 - DNA, 1638 bp, synthetic construct
  misc_feature 1..1638: note = RSV F
  source 1..1638: mol_type = other DNA
atggaacttttgatactcaaggccaatgctataactaccattttaaccgctgtcacgttttgtttcgcttctggccaaaatattaccgaagaattctatcaatctacttgctccgcagtgagcaaaggttacttatctgctttgagaacaggatggtacacatccgtgataacaattgagctctctaatataaaggagaacaagtgtaatggtaccgatgcaaaggtcaaacttataaagcaagagctggataaatacaagaatgcagtaactgagttgcaactgctgatgcaatctactcctgctactaataatagagcgagacgggaacttcccaggttcatgaactacacacttaataatgctaagaaaactaacgttacgctcagtaagaagcggaagcgccgtttcttaggatttctccttggcgttggcagtgccatagcttccggagtcgcagtctctaaagtcttgcaccttgaaggtgaagtcaacaaaattaaatcagcactgttaagcaccaataaggcagtcgtatctctgagtaatggcgtctctgtgctcacttccaaggtcctggaccttaagaactacatagacaaacaattactcccgattgtgaacaaacagagctgcagcatatcaaatatagaaaccgtgatcgaattccaacagaagaataaccgtttactggagataacgagggaattctccgtaaatgccggtgtcacaactcctgtgtctacttatatgttgaccaacagcgaactgctgtcacttataaacgacatgcccatcactaatgaccaaaagaagcttatgtccaacaacgttcaaatcgtcagacaacagagctattctatcatgtctataataaaagaggaagttcttgcatacgtcgtacaactgccattgtacggtgtgatcgacactccctgctggaaattgcatacatctccgctttgtacaacaaatacgaaggaaggcagtaatatatgtttgacccgtacagatcgtggttggtattgcgataacgccgggtccgtgtccttcttcccccaagcagaaacctgtaaagtccagtctaacagggtcttctgtgacaccatgaatagcctcaccttaccatccgaggtgaacttatgtaatgttgatattttcaatcccaagtatgactgtaaaataatgaccagtaaaaccgacgttagcagtagtgtgattacctctttgggtgcaattgttagctgttacggtaaaacaaagtgcacagcgtccaacaagaatcgcggcattatcaagacattctctaacggatgtgactatgtcagcaacaaaggagtggatacggtctctgtgggcaacaccctctactacgtcaacaaacaagaggggaagagcttatatgtgaaaggcgaacctatcatcaacttttacgatccacttgtatttcctagcgatgagttcgatgccagtatctctcaagtaaacgaaaagataaatcagtctctggcgttcattcgcaaaagcgacgagcttttaggtggggggggaagcggttacattcccgaggccccacgggacggccaggcttatgttcgcaaggatggagaatgggtgcttttgagtacgttcctttaa

SEQ ID NO: 67 - protein, 545 aa, synthetic construct
  REGION 1..545: note = RSV F
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGGGSGYIPEAPRDGQAYVRKDGEWVLLSTFL

SEQ ID NO: 68 - DNA, 2724 bp, synthetic construct
  misc_feature 1..2724: note = CMV gB
  source 1..2724: mol_type = other DNA
atggagagcaggatctggtgtttagttgtttgtgttaatctgtgcattgtttgtctgggtgcagcagttagttcaagttccacacgcggcacatcagccactcatagtcaccatagttcccataccaccagcgcagcccatagtaggtctggttcagtgtctcagcgggtgacctcaagccaaacggtctcacacggggtcaatgagacaatttataatacaacactcaagtacggggatgtagtaggtgtaaacactaccaagtatccatacagagtctgttctatggcacagggcacagatcttataaggtttgaacgaaatattgtatgtacgtccatgaaacccattaatgaggacctcgacgaaggaatcatggtggtctacaagcggaatatcgtggcccatacattcaaagtacgtgtttaccagaaagtcctcactttcagacggtcctacgcttatattcataccacatatcttctgggttccaatactgaatatgtcgcaccacctatgtgggaaatccatcatataaactcccactcacaatgctatagctcctattccagagttattgctgggacagtgttcgtggcttatcacagggatagttatgaaaataagaccatgcaactgatgccggacgattacagtaacacacactccacccgttacgtaaccgtcaaggatcagtggcactctcgcggcagtacatggctgtaccgtgagacttgcaacttgaactgtatggtcactattaccactgcaaggtccaaatatccctatcatttcttcgcgacgtctaccggagatgtggttgacatatccccgttttataacggtacaaaccgcaacgccagttacttcggagaaaatgccgataagttcttcatcttccccaactacacaatagtgtcagactttgggcgcccgaatagcgccctcgaaactcaccgcttggtcgcctttcttgagagagcagattctgtcatctcttgggacattcaggacgaaaagaacgtcacttgtcagttaaccttctgggaagcgtctgagcgcaccatcagatctgaggcggaagattcttaccattttagctcagccaagatgaccgctacctttctcagcaagaagcaagaggtgaacatgagcgatagcgctctggactgcgtaagagatgaagcaatcaacaagttacagcaaatatttaacaccagttacaaccaaacgtacgagaagtatgggaacgtatccgttttcgagacaaccggcggacttgtggtcttttggcagggtattaaacaaaagtctttagtcgaactggaaagactggcgaacagatccagcctgaacctgacacacaatcgcactaaacgttcaactgatggaaataatgcaacccatctcagcaacatggagagtgtgcacaaccttgtctacgctcagttacaattcacttatgacaccctgcgaggttacattaatcgagctctggcacaaatcgcagaggcatggtgcgttgatcaacgccggacactggaagtgtttaaggaattgagtaagattaatcccagtgcaatcttgtccgcgatctataacaagcccattgcagctcgtttcatgggagatgtcctcggattagcttcctgcgtcactattaatcaaacatccgttaaagtcctgagagacatgaatgttaaagagtcaccgggacggtgttatagtcggcccgtggtcatattcaattttgccaactctagctacgtccagtatggccaattgggtgaagataatgaaatcttattggggaatcacaggaccgaagaatgccaactccccagcttgaagatctttatagctggtaactcagcctacgagtatgtcgactatctcttcaaaaggatgatcgatctctcaagtatcagtacggtcgactcaatgatcgctctcgacatagacccgctcgaaaacactgatttccgggtgctggaactctactcacaaaaggaactccggtcatcaaatgtctttgatctggaagagatcatgcgcgagtttaattcctacaagcaaagggtcaaatacgttgaagacaaggtcgtggatcctctgcccccatatttgaaaggattagacgatctcatgtctggacttggagccgccggaaaagcagttggtgtagctatcggggcagttggcggtgctgttgctagtgtcgtggaaggggtggcgacctttctgaagaacccctttggcgcattcacgataatcttagttgcgatcgcagttgttatcattatttacttaatatatactagacaaagacgcctgtgtatgcaacccctgcaaaatctgtttccgtatctcgtatctgccgacgggaccaccgtgacgagtgggaacaccaaagacacttccctgcaagctcccccatcttacgaagaaagcgtgtataatagcggtcgaaaagggcctggacccccatcaagtgatgcctctactgctgctcccccttatactaatgaacaagcctaccaaatgctgctcgccctggttcggttagacgccgaacaacgggcgcaacagaacggtactgattcacttgacggacaaaccgggacacaagataaagggcaaaagcccaacttactggacagattgagacacagaaagaacgggtaccggcatcttaaggacagcgatgaggaagagaatgtgtga

SEQ ID NO: 69 - protein, 907 aa, synthetic construct
  REGION 1..907: note = CMV gB
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIIIYLIYTRQRRLCMQPLQNLFPYLVSADGTTVTSGNTKDTSLQAPPSYEESVYNSGRKGPGPPSSDASTAAPPYTNEQAYQMLLALVRLDAEQRAQQNGTDSLDGQTGTQDKGQKPNLLDRLRHRKNGYRHLKDSDEEENV

SEQ ID NO: 70 - DNA, 1698 bp, synthetic construct
  misc_feature 1..1698: note = H1N1 2007 Brisbane HA
  source 1..1698: mol_type = other DNA
atgaaggtgaaactgctggtgctgctgtgtaccttcacagccacctatgctgacaccatctgtattggctaccatgccaacaacagcacagacacagtggacacagtgttggagaagaatgtgacagtgacccactctgtgaacctgttggagaacagccacaatggcaaactgtgtctgctgaaaggcattgccccactccaacttggcaactgttctgtggctggctggattctgggcaaccctgagtgtgaactgctgattagcaaggagtcctggtcctacattgtggagaagccaaaccctgagaatggcacttgttaccctggacactttgctgactatgaggaactgagggaacaactttcctctgtgtcctcctttgagaggtttgagatttttccaaaggagtcctcctggccaaaccacacagtgacaggagtgtctgcctcctgtagccacaatggagagtcctccttctacaggaacctgctgtggctgacaggcaagaatggactctacccaaacctgagcaagtcctatgccaacaacaaggagaaggaggtgctggtgctgtggggagtgcatcaccctccaaacattggagaccagaaggctctctaccacacagagaatgcctatgtgtctgtggtgtccagccactacagcaggaagttcacacctgagattgccaagagaccaaaggtgagggaccaggagggcaggataaactactactggaccctgttggaacctggagacaccatcatctttgaggctaatggcaacctgattgccccaagatatgcctttgccctgagcaggggctttggctctggcatcatcaacagcaatgcccctatggacaagtgtgatgccaagtgtcagacaccacagggagccatcaactcctccctgccattccagaatgtgcatcctgtgaccattggagagtgtccaaaatatgtgaggtctgccaaactgaggatggtgacaggactgaggaacatcccaagcatccagagcaggggactgtttggagccattgctggcttcattgagggaggctggacagggatggtggatggctggtatggctaccaccaccagaatgaacagggctctggctatgctgctgaccagaagagcacccagaatgccatcaatggcatcaccaacaaggtgaactctgtgattgagaagatgaacacccagttcacagcagtgggcaaggagttcaacaaattggagaggaggatggagaacctgaacaagaaggtggatgatggcttcattgacatctggacctacaatgctgaactgctggtgctgttggagaatgagaggaccctggacttccatgacagcaatgtgaagaacctctatgagaaggtgaagagccaacttaaaaacaatgccaaggagattggcaatggctgttttgagttctaccacaagtgtaatgatgagtgtatggagtctgtgaagaatggcacctatgactacccaaaatactctgaggagagcaaactgaacagggagaagattgatggagtgaaattggagagtatgggagtctaccagattctggctatctacagcacagtggcatcctccctggtgctgctggtgtccctgggagccatctccttctggatgtgtagcaatggctccctccaatgtaggatttgtatttaa

SEQ ID NO: 71 - protein, 565 aa, synthetic construct
  REGION 1..565: note = H1N1 2007 Brisbane HA
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGDQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 72 - protein, 1270 aa, synthetic construct
  REGION 1..1270: note = SARS-CoV-2 Omicron BA.1
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 73 - protein, 1270 aa, synthetic construct
  REGION 1..1270: note = SARS-CoV-2 Omicron BA.2
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 74 - protein, 1271 aa, synthetic construct
  REGION 1..1271: note = SARS-CoV-2 Delta Variant
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGEENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 75 - protein, 181 aa, synthetic construct
  REGION 1..181: note = Monkeypox virus A35R Amino Acid Seq.
MMTPENDEEQTSVFSATVYGDKIQGKNKRKRVIGLCIRISMVISLLSMITMSAFLIVRLNQCMSANEAAITDSAVAVAAASSTHRKVASSTTQYDHKESCNGLYYQGSCYILHSDYKSFEDAKANCAAESSTLPNKSDVLTTWLIDYVEDTWGSDGNPITKTTSDYQDSDVSQEVRKYFCT

SEQ ID NO: 76 - protein, 324 aa, synthetic construct
  REGION 1..324: note = Monkeypox virus H3L Amino Acid Seq.
MAAVKTPVIVVPVIDRPPSETFPNVHEHINDQKFDDVKDNEVMQEKRDVVIVNDDPDHYKDYVFIQWTGGNIRDDDKYTHFFSGFCNTMCTEETKRNIARHLALWDSKFFTELENKNVEYVVIIENDNVIEDITFLRPVLKAIHDKKIDILQMREIITGNKVKTELVIDKDHAIFTYTGGYDVSLSAYIIRVTTALNIVDEIIKSGGLSSGFYFEIARIENEMKINRQIMDNSAKYVEHDPRLVAEHRFETMKPNFWSRIGTVAAKRYPGVMYTFTTPLISFFGLFDINVIGLIVILFIMFMLIFNVKSKLLWFLTGTFVTAFI

SEQ ID NO: 77 - protein, 152 aa, synthetic construct
  REGION 1..152: note = Monkeypox virus L1R Amino Acid Seq.
MDHNQYLLTMFFADDDSFFKYFASQDDESSLSDILQITQYLDFLLLLLIQSKNKLEAVGHCYESLSEEYRQLTKFTDSQDFKKLFNKVPIVTDGRVKLNKGYLFDFVISLMRFKKESALATTAIDPVRYIDPRRDIAFSNVMDILKSNKVEQ

SEQ ID NO: 78 - protein, 712 aa, synthetic construct
  REGION 1..712: note = Clostridioides difficile 630 spore coat protein: peroxiredoxin/chitinase Amino Acid Seq.
MIYMPNLPSLGSKAPDFKANTTNGPIRLSDYKGNWIVLFSHPGDFTPVCTTEFLCFAKYYDEFKKRNTELIGLSVDSNSSHLAWMYNISLLTGVEIPFPIIEDRDMRIAKLYGMISKPMSDTSTVRSVFIIDNNQILRTILYYPLTTGRNIPEILRIVDALQTSDRDNIVTPANWFPGMPVILPYPKNYKELKNRVNSCNKKYSCMDWYLCFVPDNYNDEEVSKKIDNTCSWKKEHTKNIENECNCEHEHHDYLNKALDCKQEHKTDIKDDCNHEKKHTKNTNKVHNSKQDKFKDKSCDEMNFNYDKDESCDKINSSYNKEDSSYEDFYKHNYKNYDYTSEKNTKKIAMKTLKDSKKLVRPQITDPYNPIVENANCPDINPIVAEYVLGNPTNVDAQLLDAVIFAFAEIDQSGNLFIPYPRFLNQLLALKGEKPSLKVIVAIGGWGAEGFSDAALTPTSRYNFARQVNQMINEYALDGIDIDWEYPGSSASGITSRPQDRENFTLLLTAIRDVIGDDKWLSVAGTGDRGYINSSAEIDKIAPIIDYFNLMSYDFTAGETGPNGRKHQANLFDSDLSLPGYSVDAMVRNLENAGMPSEKILLGIPFYGRLGATITRTYDELRRDYINKNGYEYRFDNTAQVPYLVKDGDFAMSYDDALSIFLKTQYVLRNCLGGVFSWTSTYDQANILARTMSIGINDPEVLKEELEGIYGQF

SEQ ID NO: 79 - protein, 290 aa, synthetic construct
  REGION 1..290: note = Clostridioides difficile 630 flagellin C Amino Acid Seq.
MRVNTNVSALIANNQMGRNVNGQSKSMEKLSSGVRIKRAADDAAGLAISEKMRAQIKGLDQAGRNVQDGISVVQTAEGSLEETGNILQRMRTLSLQSANEINNTEEREKIADELTQLKDEIERISSSTEFNGKKLLDGTSSTIRLQVGASYGTNVSGTSNNNNEIKIQLVNTASIMASAGITTASIGSMKAGGTTGTDAAKTMVSSLDAALKSLNSSRAKLGAQQNRLESTQNNLNNTLENVTAAESRIRDTDVASEMVNLSKMNILVQASQSMLAQANQQPQGVLQLLG

SEQ ID NO: 80 - protein, 190 aa, synthetic construct
  REGION 1..190: note = Clostridioides difficile Surface layer protein A (Fragment) Amino Acid Seq.
GDGDYVDFLITSPGQGDKITTSKLVALKDLTGASADAIIAGTSSADGVVTNTGAASGSTETNSAGTKLAMSAIFDTAYTDSSETAVKITIKADMNDTKFGKAGETTYSTGLTFEDGSTEKIVKLGDSDIIDITKALKLTVVPGSKATVKFAEKTPSASVQPVITKLRIINAKEETIDIDASSSKTAQDLA

SEQ ID NO: 81 - protein, 641 aa, synthetic construct
  REGION 1..641: note = Epstein-Barr virus (strain B95-8) EBNA1_EBVB9 Epstein-Barr nuclear antigen 1 Amino Acid Seq
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGAGAGAGGAGAGGAGAGGGAGAGGGAGGAGGAGGAGAGGGAGAGGGAGGAGGAGAGGGAGAGGGAGGAGAGGGAGGAGGAGAGGGAGAGGGAGGAGAGGGAGGAGGAGAGGGAGAGGAGGAGGAGAGGAGAGGGAGGAGGAGAGGAGAGGAGAGGAGAGGAGGAGAGGAGGAGAGGAGGAGAGGGAGGAGAGGGAGGAGAGGAGGAGAGGAGGAGAGGAGGAGAGGGAGAGGAGAGGGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 82 - protein, 857 aa, synthetic construct
  REGION 1..857: note = Epstein-Barr virus (strain B95-8) GB_EBVB9 Envelope glycoprotein B Amino Acid Seq.
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 83 - protein, 706 aa, synthetic construct
  REGION 1..706: note = Epstein-Barr virus (strain B95-8) GH_EBVB9 Envelope glycoprotein H Amino Acid Seq.
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 84 - protein, 907 aa, synthetic construct
  REGION 1..907: note = Epstein-Barr virus (strain B95-8) GP350_EBVB9 Envelope glycoprotein GP350 Amino Acid Seq.
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 85 - protein, 386 aa, synthetic construct
  REGION 1..386: note = Epstein-Barr virus (strain B95-8) Latent membrane protein 1 Amino Acid Seq.
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 86 - protein, 497 aa, synthetic construct
  REGION 1..497: note = Epstein-Barr virus (strain B95-8) Latent membrane protein 2 Amino Acid Seq.
MGSLEMVPMGAGPPSPGGDPDGYDGGNNSQYPSASGSSGNTPTPPNDEERESNEEPPPPYEDPYWGNGDRHSDYQPLGTQDQSLYLGLQHDGNDGLPPPPYSPRDDSSQHIYEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 87 - protein, 273 aa, synthetic construct
  REGION 1..273: note = Neisseria meningitidis Factor H-binding protein Amino Acid Seq.
MNRTTFCCLSLTTALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPGGKAEYHGKAFSSDDPNGRLHYSIDFTKKQGYGRIEHLKTPEQNVELASAELKADEKSHAVILGDTRYGSEEKGTYHLALFGDRAQEIAGSATVKIREKVHEIGIAGKQ

SEQ ID NO: 88 - protein, 405 aa, synthetic construct
  REGION 1..405: note = Neisseria meningitidis serogroup B Neisseria adhesin A Amino Acid Seq.
MKHFPSKVLTTAILATFCSGALAATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVGRFNVTAAVGGYKSESAVAIGTGFRFTENFAAKAGVAVGTSSGSSAAYHVGVNYEW

SEQ ID NO: 89 - protein, 486 aa, synthetic construct
  REGION 1..486: note = Neisseria meningitidis Neisserial heparin binding antigen Amino Acid Seq.
MFERSVIAMACIFALSACGGGGGGSPDVKSADTLSKPAAPVVSEKETEAKEDAPQAGSQGQGAPSAQGSQDMAAVSEENTGNGGAATADNPKNEDEGAQDDMPQNAADTDSLTPNHTPASNMPAVNMENQAPDTGESVQPANQPDMANAADGMQGDDPSAGGQNAGNTAAQGANQAGNNQAAGSSDPIPASNPATTNSGGDFGRTNVANGIKLDGPSQNITLTHCKDTVCGSNFLAEEAPSKSEFESLDDSGRINKYKKDGQDKFTNLVATEVKANGTNKYVIIYKDKSTSSARVRRSARSRRSLPAEMPLIPVNQADTLIVDGEAVSLTGHSGNIFAPEGNYRYLTYGAEKLSGGSYALRVQGEPAKGEMLAGTAVYNGEVLHFHTENGRPYPTRGRFAAKVDFGSKSVDGIIDSGDDLHMGTQKFKAAIDGNGFKGTWTENGGGDVSGRFYGPAGEEVAGKYSYRPTDAEKGGFGVFAGKKEQD

SEQ ID NO: 90 - protein, 110 aa, synthetic construct
  REGION 1..110: note = Vaccinia virus (strain Western Reserve) Protein A27 Amino Acid Seq.
MDGTLFPGDDDLAIPATEFFSTKAAKKPEAKREAIVKADEDDNEETLKQRLTNLEKKITNVTTKFEQIEKCCKRNDEVLFRLENHAETLRAAMISLAKKIDVQTGRRPYE

SEQ ID NO: 91 - protein, 185 aa, synthetic construct
  REGION 1..185: note = Vaccinia virus (strain Western Reserve) EEV membrane phosphoglycoprotein Amino Acid Seq.
MMTPENDEEQTSVFSATVYGDKIQGKNKRKRVIGLCIRISMVISLLSMITMSAFLIVRLNQCMSANEAAITDAAVAVAAASSTHRKVASSTTQYDHKESCNGLYYQGSCYILHSDYQLFSDAKANCTAESSTLPNKSDVLITWLIDYVEDTWGSDGNPITKTTSDYQDSDVSQEVRKYFCVKTMN

SEQ ID NO: 92 - protein, 317 aa, synthetic construct
  REGION 1..317: note = Vaccinia virus B5R (Fragment) Amino Acid Seq.
MKTISVVTLLCVLPAVVYSTCTVPTMNNAKLTSTETSFNDKQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDMPSLSNGLISGSTFSIGGVIHLSCKSGFTLTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 93 - protein, 324 aa, synthetic construct
  REGION 1..324: note = Vaccinia virus Envelope protein H3 Amino Acid Seq.
MAAVKTPVIVVPVIDRPPSETFPNVHEHINDQKFDDVKDNEVMPEKRNVVVVKDDPDHYKDYAFIQWTGGNIRNDDKYTHFFSGFCNTMCTEETKRNIARHLALWDSNFFTELENKKVEYVVIVENDNVIEDITFLRPVLKAMHDKKIDILQMREIITGNKVKTELVMDKNHAIFTYTGGYDVSLSAYIIRVTTALNIVDEIIKSGGLSSGFYFEIARIENEMKINRQILDNAAKYVEHDPRLVAEYRFENMKPNFWSRIGTAAAKRYPGVMYAFTTPLISFFGLFDINVIGLIVILFIMFMLIFNVKSKLLWFLTGTFVTAFI

SEQ ID NO: 94 - protein, 250 aa, synthetic construct
  REGION 1..250: note = Vaccinia virus (strain Western Reserve) IMV membrane protein Amino Acid Seq.
MGAAASIQTTVNTLSERISSKLEQEANASAQTKCDIEIGNFYIRQNHGCNLTVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPKQVAGTGVQFYMIVIGVIILAALFMYYAKRMLFTSTNDKIKLILANKENVHWTTYMDTFFRTSPMVIATTDMQN

SEQ ID NO: 95 - protein, 546 aa, synthetic construct
  REGION 1..546: note = Nipah virus Fusion glycoprotein F0 Amino Acid Seq.
MAVILNKRYYSNLLILILMISECSVGILHYEKLSKIGLVKGITRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSGYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 96 - protein, 602 aa, synthetic construct
  REGION 1..602: note = Nipah virus Glycoprotein G Amino Acid Seq.
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 97 - protein, 623 aa, synthetic construct
  REGION 1..623: note = Varicella-zoster virus (strain Dumas) Envelope glycoprotein E Amino Acid Seq.
MGTVNKPVVGVLMGFGIITGTLRITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 98 - protein, 365 aa, synthetic construct
  REGION 1..365: note = Human papillomavirus type 16 Regulatory protein E2 Amino Acid Seq.
METLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDANTLKCLRYRFKKHCTLYTAVSSTWHWTGHNVKHKSAIVTLTYDSEWQRDQFLSQVKIPKTITVSTGFMSI

SEQ ID NO: 99 - protein, 158 aa, synthetic construct
  REGION 1..158: note = Human papillomavirus type 16 Protein E6 Amino Acid Seq.
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 100 - protein, 98 aa, synthetic construct
  REGION 1..98: note = Human papillomavirus type 16 Protein E7 Amino Acid Seq.
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 101 - protein, 365 aa, synthetic construct
  REGION 1..365: note = Human papillomavirus type 18 Regulatory protein E2 Amino Acid Seq.
MQTPKETLSERLSCVQDKIIDHYENDSKDIDSQIQYWQLIRWENAIFFAAREHGIQTLNHQVVPAYNISKSKAHKAIELQMALQGLAQSAYKTEDWTLQDTCEELWNTEPTHCFKKGGQTVQVYFDGNKDNCMTYVAWDSVYYMTDAGTWDKTATCVSHRGLYYVKEGYNTFYIEFKSECEKYGNTGTWEVHFGNNVIDCNDSMCSTSDDTVSATQLVKQLQHTPSPYSSTVSVGTAKTYGQTSAATRPGHCGLAEKQHCGPVNPLLGAATPTGNNKRRKLCSGNTTPIIHLKGDRNSLKCLRYRLRKHSDHYRDISSTWHWTGAGNEKTGILTVTYHSETQRTKFLNTVAIPDSVQILVGYMTM

SEQ ID NO: 102 - protein, 158 aa, synthetic construct
  REGION 1..158: note = Human papillomavirus type 18 Protein E6 Amino Acid Seq.
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 103 - protein, 105 aa, synthetic construct
  REGION 1..105: note = Human papillomavirus type 18 Protein E7 Amino Acid Seq.
MHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 104 - protein, 368 aa, synthetic construct
  REGION 1..368: note = Human papillomavirus type 6a Regulatory protein E2 Amino Acid Seq.
MEAIAKRLDACQEQLLELYEENSTDLNKHVLHWKCMRHESVLLYKAKQMGLSHIGMQVVPPLKVSEAKGHNAIEMQMHLESLLKTEYSMEPWTLQETSYEMWQTPPKRCFKKRGKTVEVKFDGCANNTMDYVVWTDVYVQDTDSWVKVHSMVDAKGIYYTCGQFKTYYVNFVKEAEKYGSTKQWEVCYGSTVICSPASVSSTTQEVSIPESTTYTPAQTSTPVSSSTQEDAVQTPPRKRARGVQQSPCNALCVAHIGPVDSGNHNLITNNHDQHQRRNNSNSSATPIVQFQGESNCLKCFRYRLNDKHRHLFDLISSTWHWASPKAPHKHAIVTVTYHSEEQRQQFLNVVKIPPTIRHKLGFMSLHLL

SEQ ID NO: 105 - protein, 150 aa, synthetic construct
  REGION 1..150: note = Human papillomavirus type 6a Protein E6 Amino Acid Seq.
MESANASTSATTIDQLCKTFNLSMHTLQINCVFCKNALTTAEIYSYAYKQLKVLFRGGYPYAACACCLEFHGKINQYRHFDYAGYATTVEEETKQDILDVLIRCYLCHKPLCEVEKVKHILTKARFIKLNCTWKGRCLHCWTTCMEDMLP

SEQ ID NO: 106 - protein, 98 aa, synthetic construct
  REGION 1..98: note = Human papillomavirus type 6a Protein E7 Amino Acid Seq.
MHGRHVTLKDIVLDLQPPDPVGLHCYEQLVDSSEDEVDEVDGQDSQPLKQHFQIVTCCCGCDSNVRLVVQCTETDIREVQQLLLGTLDIVCPICAPKT

SEQ ID NO: 107 - protein, 367 aa, synthetic construct
  REGION 1..367: note = Human papillomavirus 11 Regulatory protein E2 Amino Acid Seq.
MEAIAKRLDACQDQLLELYEENSIDIHKHIMHWKCIRLESVLLHKAKQMGLSHIGLQVVPPLTVSETKGHNAIEMQMHLESLAKTQYGVEPWTLQDTSYEMWLTPPKRCFKKQGNTVEVKFDGCEDNVMEYVVWTHIYLQDNDSWVKVTSSVDAKGIYYTCGQFKTYYVNFNKEAQKYGSTNHWEVCYGSTVICSPASVSSTVREVSIAEPTTYTPAQTTAPTVSACTTEDGVSAPPRKRARGPSTNNTLCVANIRSVDSTINNIVTDNYNKHQRRNNCHSAATPIVQLQGDSNCLKCFRYRLNDKYKHLFELASSTWHWASPEAPHKNAIVTLTYSSEEQRQQFLNSVKIPPTIRHKVGFMSLHLL

SEQ ID NO: 108 - protein, 150 aa, synthetic construct
  REGION 1..150: note = Human papillomavirus 11 Protein E6 Amino Acid Seq.
MESKDASTSATSIDQLCKTFNLSLHTLQIQCVFCRNALTTAEIYAYAYKNLKVVWRDNFPFAACACCLELQGKINQYRHFNYAAYAPTVEEETNEDILKVLIRCYLCHKPLCEIEKLKHILGKARFIKLNNQWKGRCLHCWTTCMEDLLP

SEQ ID NO: 109 - protein, 98 aa, synthetic construct
  REGION 1..98: note = Human papillomavirus 11 Protein E7 Amino Acid Seq.
MHGRLVTLKDIVLDLQPPDPVGLHCYEQLEDSSEDEVDKVDKQDAQPLTQHYQILTCCCGCDSNVRLVVECTDGDIRQLQDLLLGTLNIVCPICAPKP

SEQ ID NO: 110 - protein, 3011 aa, synthetic construct
  REGION 1..3011: note = Hepatitis C virus genotype 1a Genome polyprotein Amino Acid Seq.
MSTNPKPQKKNKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSTGLYHVTNDCPNSSIVYEAADAILHTPGCVPCVREGNASRCWVAMTPTVATRDGKLPATQLRRHIDLLVGSATLCSALYVGDLCGSVFLVGQLFTFSPRRHWTTQGCNCSIYPGHITGHRMAWDMMMNWSPTTALVMAQLLRIPQAILDMIAGAHWGVLAGIAYFSMVGNWAKVLVVLLLFAGVDAETHVTGGSAGHTVSGFVSLLAPGAKQNVQLINTNGSWHLNSTALNCNDSLNTGWLAGLFYHHKFNSSGCPERLASCRPLTDFDQGWGPISYANGSGPDQRPYCWHYPPKPCGIVPAKSVCGPVYCFTPSPVVVGTTDRSGAPTYSWGENDTDVFVLNNTRPPLGNWFGCTWMNSTGFTKVCGAPPCVIGGAGNNTLHCPTDCFRKHPDATYSRCGSGPWITPRCLVDYPYRLWHYPCTINYTIFKIRMYVGGVEHRLEAACNWTRGERCDLEDRDRSELSPLLLTTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGKWVPGAVYTFYGMWPLLLLLLALPQRAYALDTEVAASCGGVVLVGLMALTLSPYYKRYISWCLWWLQYFLTRVEAQLHVWIPPLNVRGGRDAVILLMCAVHPTLVFDITKLLLAVFGPLWILQASLLKVPYFVRVQGLLRFCALARKMIGGHYVQMVIIKLGALTGTYVYNHLTPLRDWAHNGLRDLAVAVEPVVFSQMETKLITWGADTAACGDIINGLPVSARRGREILLGPADGMVSKGWRLLAPITAYAQQTRGLLGCIITSLTGRDKNQVEGEVQIVSTAAQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGIFRAAVCTRGVAKAVDFIPVENLETTMRSPVFTDNSSPPVVPQSFQVAHLHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEITLTHPVTKYIMTCMSADLEVVTSTWVLVGGVLAALAAYCLSTGCVVIVGRVVLSGKPAIIPDREVLYREFDEMEECSQHLPYIEQGMMLAEQFKQKALGLLQTASRQAEVIAPAVQTNWQKLETFWAKHMWNFISGIQYLAGLSTLPGNPAIASLMAFTAAVTSPLTTSQTLLFNILGGWVAAQLAAPGAATAFVGAGLAGAAIGSVGLGKVLIDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYKGVWRVDGIMHTRCHCGAEITGHVKNGTMRIVGPRTCRNMWSGTFPINAYTTGPCTPLPAPNYTFALWRVSAEEYVEIRQVGDFHYVTGMTTDNLKCPCQVPSPEFFTELDGVRLHRFAPPCKPLLREEVSFRVGLHEYPVGSQLPCEPEPDVAVLTSMLTDPSHITAEAAGRRLARGSPPSVASSSASQLSAPSLKATCTANHDSPDAELIEANLLWRQEMGGNITRVESENKVVILDSFDPLVAEEDEREISVPAEILRKSRRFAQALPVWARPDYNPPLVETWKKPDYEPPVVHGCPLPPPKSPPVPPPRKKRTVVLTESTLSTALAELATRSFGSSSTSGITGDNTTTSSEPAPSGCPPDSDAESYSSMPPLEGEPGDPDLSDGSWSTVSSEANAEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVTHINSVWKDLLEDNVTPIDTTIMAKNEVFCVQPEKGGRKPARLIVFPDLGVRVCEKMALYDVVTKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLARGGRAAICGKYLFNWAVRTKLKLTPIAAAGQLDLSGWFTAGYSGGDIYHSVSHARPRWIWFCLLLLAAGVGIYLLPNR

SEQ ID NO: 111 - protein, 3010 aa, synthetic construct
  REGION 1..3010: note = Hepatitis C virus genotype 1b Genome polyprotein Amino Acid Seq.
MSTNPKPQRKTKRNTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGVARALAHGVRVVEDGVNYATGNLPGCSFSIFLLALLSCLTIPASAYEVRNVSGIYHVTNDCSNSSIVYEAADMIMHTPGCVPCVREGNSSRCWVALTPTLAARNASVPTTAIRRHVDLLVGAAAFCSAMYVGDLCGSVFLVSQLFTFSPRRHETIQDCNCSIYPGHVSGHRMAWDMMMNWSPTTALVVSQLLRIPQAIVDMVAGAHWGVLAGLAYYSMVGNWAKVLIVMLLFAGVDGETRVTGGQIARNAYSLTTLFSSGSAQNIQLINTNGSWHINRTALNCNDSLNTGFLAALFYTHKFNASGCPERLASCRPIDKFDQGWGPITYAEQGGQDQRPYCWHYAPKPCGIVSASKVCGPVYCFTPSPVVVGTTDRFGVPTYSWGENETDVLLLNNTRPPQGNWFGCTWMNGTGFTKTCGGPPCNIGGGGNNTLTCPTDCFRKHPAATYTKCGSGPWLTPRCLVDYPYRLWHYPCTANFTIFKVRMYVGGVEHRLDAACNWTRGERCNLEDRDRLELSPLLLSTTEWQVLPCSFTTLPALSTGLIHLHQNIVDVQYLYGIGSAVVSFAIKWDYIVILFLLLADARVCACLWMMLLIAQAEAALENLVVLNAASVAGAHGILSFLVFFCAAWYIKGKLVPGAAYAFYGVWPLLLLLLALPPRAYAMEREMAASCGGAVFVGLVLLTLSPYYKEFLARLIWWLQYFITRAEAHLQVWIPPLNIRGGRDAIILLACVVHPELIFDITKLLLAILGPLMVLQASITQVPYFVRAQGLIRACMLVRKVAGGHYVQMAFVKLTALTGTYVYDHLTPLRDWAHAGLRDLAVAVEPVVFSDMETKVITWGADTAACGDIILGLPVSARRGREILLGPADSLEGQGWRLLAPITAYSQQTRGLLGCIITSLTGRDKNQVEGEVQVVSTATQSFLATCVNGACWTVFHGAGSKTLAGPKGPITQMYTNVDLDLVGWQAPPGSRSLTPCTCGSSDLYLVTRHADVIPVRRRGDSRGSLLSPRPVSYLKGSSGGPLLCPSRHAVGIFRAAVCTRGVAKAVDFIPVESMETTMRSPVFTDNSSPPAVPQTFQVAHLHAPTGSGKSTKVPAAYAAQGYKVLVLNPSVAATLGFGAYMSKAHGIDPNIRTGVRAITTGAPITYSTYGKFLADGGCSGGAYDIIICDECHSTDSTTILGIGTVLDQAETAGARLVVLATATPPGSVTVPHPNIEEVALSNAGEIPFYGKAIPIEVIKGGRHLIFCHSKKKYDELAAKLSALGLNAVAYYRGLDVSVIPTNGDVVVVATDALMTGFTGDFDSVIDCNTCVTQTVDFSLDPTFTIETTTVPQDAVARSQRRGRTGRGRRGIYRFVTPGERPSGMFDSSVLCECYDAGCAWYELTPAETSVRLRAYLNTPGLPVCQDHLEFWESVSTGLTHIDAHFLSQTKQAGDNFPYLVAYQATVCARAQAPPPSWDQMWKCLIRLKPTLHGPTPLLYRLGAVQNEITLTHPMTKFIMACMSADLEVVTSTWVLVGGVLAALAAYCLTTGSVVIVGRIILSGRPAVIPDREVLYREFDEMEECASHLPYIEQGMQLAEQFKQKALGLLQTATKQAEAAAPVVESKWRALETFWAKHMWNFISGIQYLAGLSTLPGNPAIASLMAFTASITSPLSTQNTLLFNIWGGWVAAQLAPPSAASAFVGAGIAGAAVGSIGLGKVLVDILAGYGAGVAGALVAFKIMSGEVPSTEDLVNLLPAILSPGALVVGVVCAAILRRHVGPGEGAVQWMNRLIAFASRGNHVSPAHYVPESDAAARVTQILSGLTITQLLKRLHHWINEDCSTPCSGSWLRDVWDWICTVLTDFKTWLQSKLLPRLPGVPFFSCQRGYKGVWRGDGIMQTTCPCGAQITGHVKNGSMRIVGPKTCSSTWHGTFPINAYTTGPCAPSPAPNYSRALWRVAAEEYVEVTRVGDFHYVTGMTTDNVKCPCQVPAPEFFTEVDGVRLHRYAPACKPLLREEVTFQVGLNQYLVGSQLPCEPEPDVAVLTSMLTDPSHITAETAKRRLARGSPPSLASSSASQLSAPSLKATCTTHHDSPDVDLIEANLLWRQEMGGNITRVESENKVVILDSFDPLRAEEDEREPSVAAEILRKTKRFPPAMPIWARPDYNPPLLESWKDPDYVPPVVHGCPLPPTKAPPIPPPRRKRTVVLTESTVSSALAELATKTFGSSGSSAVDSGTATAPPDQASDDGDQGSDVESYSSMPPLEGEPGDPDLSDGSWSTVSEEAGEDVICCSMSYTWTGALITPCAAEESKLPINPLSNSLLRHHNMVYATTSRSAGLRQKKVTFDRLQVLDDHYRDVLKEMKAKASTVKAKLLSIEEACKLTPPHSARSKFGYGAKDVRNLSSKAVNHIRSVWKDLLEDTETPIDTTVMAKSEVFCVQPEKGGRKPARLIVFPDLGVRVCEKMALYDVVSTLPQAVMGSSYGFQYSPGQRVEFLVNAWKSKKCPMGFSYDTRCFDSTVTESDIRVEESIYQCCDLAPEARQAIKSLTERLYIGGPLTNSKGQNCGYRRCRASGVLTTSCGNTLTCYLKASAACRAAKLRDCTMLVNGDDLVVICESAGTQEDEANLRVFTEAMTRYSAPPGDPPRPEYDLELITSCSSNVSVAHDASGKRVYYLTRDPSTPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRAKLLSQGGRAATCGKYLFNWAVRTKLKLTPIPAASQLDLSSWFVAGYSGGDIYHSLSRARPRWFMLCLLLLSVGVGIYLLPNR

SEQ ID NO: 112 - protein, 3033 aa, synthetic construct
  REGION 1..3033: note = Hepatitis C virus genotype 2a Genome polyprotein Amino Acid Seq.
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNTSRCWIPVSPNVAVQQPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIIGGAHWGVMFGLAYFSMQGAWAKVVVILLLAAGVDAQTHTVGGSTAHNARTLTGMFSLGARQKIQLINTNGSWHINRTALNCNDSLHTGFLASLFYTHSFNSSGCPERMSACRSIEAFRVGWGALQYEDNVTNPEDMRPYCWHYPPRQCGVVSASSVCGPVYCFTPSPVVVGTTDRLGAPTYTWGENETDVFLLNSTRPPQGSWFGCTWMNSTGYTKTCGAPPCRIRADFNASMDLLCPTDCFRKHPDTTYIKCGSGPWLTPRCLIDYPYRLWHYPCTVNYTIFKIRMYVGGVEHRLTAACNFTRGDRCNLEDRDRSQLSPLLHSTTEWAILPCTYSDLPALSTGLLHLHQNIVDVQFMYGLSPALTKYIVRWEWVVLLFLLLADARVCACLWMLILLGQAEAALEKLVVLHAASAASCNGFLYFVIFFVAAWYIKGRVVPLATYSLTGLWSFGLLLLALPQQAYAYDASVHGQIGAALLVLITLFTLTPGYKTLLSRFLWWLCYLLTLAEAMVQEWAPPMQVRGGRDGIIWAVAIFCPGVVFDITKWLLAVLGPAYLLKGALTRVPYFVRAHALLRMCTMVRHLAGGRYVQMVLLALGRWTGTYIYDHLTPMSDWAANGLRDLAVAVEPIIFSPMEKKVIVWGAETAACGDILHGLPVSARLGREVLLGPADGYTSKGWSLLAPITAYAQQTRGLLGTIVVSMTGRDKTEQAGEIQVLSTVTQSFLGTTISGVLWTVYHGAGNKTLAGSRGPVTQMYSSAEGDLVGWPSPPGTKSLEPCTCGAVDLYLVTRNADVIPARRRGDKRGALLSPRPLSTLKGSSGGPVLCPRGHAVGVFRAAVCSRGVAKSIDFIPVETLDIVTRSPTFSDNSTPPAVPQTYQVGYLHAPTGSGKSTKVPVAYAAQGYKVLVLNPSVAATLGFGAYLSKAHGINPNIRTGVRTVTTGAPITYSTYGKFLADGGCAGGAYDIIICDECHAVDSTTILGIGTVLDQAETAGVRLTVLATATPPGSVTTPHPNIEEVALGQEGEIPFYGRAIPLSYIKGGRHLIFCHSKKKCDELAAALRGMGLNAVAYYRGLDVSVIPTQGDVVVVATDALMTGFTGDFDSVIDCNVAVTQVVDFSLDPTFTITTQTVPQDAVSRSQRRGRTGRGRLGIYRYVSTGERASGMFDSVVLCECYDAGAAWYELTPAETTVRLRAYFNTPGLPVCQDHLEFWEAVFTGLTHIDAHFLSQTKQSGENFAYLTAYQATVCARAKAPPPSWDVMWKCLTRLKPTLVGPTPLLYRLGSVTNEVTLTHPVTKYIATCMQADLEVMTSTWVLAGGVLAAVAAYCLATGCVCIIGRLHVNQRAVVAPDKEVLYEAFDEMEECASRAALIEEGQRIAEMLKSKIQGLLQQASKQAQDIQPAVQASWPKVEQFWAKHMWNFISGIQYLAGLSTLPGNPAVASMMAFSAALTSPLSTSTTILLNILGGWLASQIAPPAGATGFVVSGLVGAAVGSIGLGKVLVDILAGYGAGISGALVAFKIMSGEKPSMEDVVNLLPGILSPGALVVGVICAAILRRHVGPGEGAVQWMNRLIAFASRGNHVAPTHYVTESDASQRVTQLLGSLTITSLLRRLHNWITEDCPIPCSGSWLRDVWDWVCTILTDFKNWLTSKLFPKMPGLPFISCQKGYKGVWAGTGIMTTRCPCGANISGNVRLGSMRITGPKTCMNIWQGTFPINCYTEGQCVPKPAPNFKIAIWRVAASEYAEVTQHGSYHYITGLTTDNLKVPCQLPSPEFFSWVDGVQIHRFAPIPKPFFRDEVSFCVGLNSFVVGSQLPCDPEPDTDVLTSMLTDPSHITAETAARRLARGSPPSEASSSASQLSAPSLRATCTTHGKAYDVDMVDANLFMGGDVTRIESESKVVVLDSLDPMVEERSDLEPSIPSEYMLPKKRFPPALPAWARPDYNPPLVESWKRPDYQPATVAGCALPPPKKTPTPPPRRRRTVGLSESSIADALQQLAIKSFGQPPPSGDSGLSTGADAADSGSRTPPDELALSETGSISSMPPLEGEPGDPDLEPEQVELQPPPQGGVVTPGSGSGSWSTCSEEDDSVVCCSMSYSWTGALITPCSPEEEKLPINPLSNSLLRYHNKVYCTTSKSASLRAKKVTFDRMQALDAHYDSVLKDIKLAASKVTARLLTLEEACQLTPPHSARSKYGFGAKEVRSLSGRAVNHIKSVWKDLLEDTQTPIPTTIMAKNEVFCVDPTKGGKKAARLIVYPDLGVRVCEKMALYDITQKLPQAVMGASYGFQYSPAQRVEFLLKAWAEKKDPMGFSYDTRCFDSTVTERDIRTEESIYRACSLPEEAHTAIHSLTERLYVGGPMFNSKGQTCGYRRCRASGVLTTSMGNTITCYVKALAACKAAGIIAPTMLVCGDDLVVISESQGTEEDERNLRAFTEAMTRYSAPPGDPPRPEYDLELITSCSSNVSVALGPQGRRRYYLTRDPTTPIARAAWETVRHSPVNSWLGNIIQYAPTIWARMVLMTHFFSILMAQDTLDQNLNFEMYGAVYSVSPLDLPAIIERLHGLDAFSLHTYTPHELTRVASALRKLGAPPLRAWKSRARAVRASLISRGGRAAVCGRYLFNWAVKTKLKLTPLPEARLLDLSSWFTVGAGGGDIYHSVSRARPRLLLLGLLLLFVGVGLFLLPAR

SEQ ID NO: 113 - protein, 3021 aa, synthetic construct
  REGION 1..3021: note = Hepatitis C virus genotype 3a Genome polyprotein Amino Acid Seq.
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIMAGAHWGILAGLAYYSMQGNWAKVAIIMVMFSGVDAHTYTTGGTASRHTQAFAGLFDIGPQQKLQLVNTNGSWHINSTALNCNESINTGFIAGLFYYHKFNSTGCPQRLSSCKPITFFRQGWGPLTDANITGPSDDRPYCWHYAPRPCDIVPASSVCGPVYCFTPSPVVVGTTDARGVPTYTWGENEKDVFLLKSQRPPSGRWFGCSWMNSTGFLKTCGAPPCNIYGGEGNPHNESDLFCPTDCFRKHPETTYSRCGAGPWLTPRCMVDYPYRLWHYPCTVDFRLFKVRMFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQTEAALENLVTLNAVAAAGTHGIGWYLVAFCAAWYVRGKLVPLVTYSLTGLWSLALLVLLLPQRAYAWSGEDSATLGAGVLVLFGFFTLSPWYKHWIGRLMWWNQYTICRCESALHVWVPPLLARGSRDGVILLTSLLYPSLIFDITKLLMAVLGPLYLIQATITTTPYFVRAHVLVRLCMLVRSVIGGKYFQMIILSIGRWFNTYLYDHLAPMQHWAAAGLKDLAVATEPVIFSPMEIKVITWGADTAACGDILCGLPVSARLGREVLLGPADDYREMGWRLLAPITAYAQQTRGLLGTIVTSLTGRDKNVVTGEVQVLSTATQTFLGTTVGGVIWTVYHGAGSRTLAGAKHPALQMYTNVDQDLVGWPAPPGAKSLEPCACGSSDLYLVTRDADVIPARRRGDSTASLLSPRPLACLKGSSGGPVMCPSGHVAGIFRAAVCTRGVAKSLQFIPVETLSTQARSPSFSDNSTPPAVPQSYQVGYLHAPTGSGKSTKVPAAYVAQGYNVLVLNPSVAATLGFGSFMSRAYGIDPNIRTGNRTVTTGAKLTYSTYGKFLADGGCSGGAYDVIICDECHAQDATSILGIGTVLDQAETAGVRLTVLATATPPGSITVPHSNIEEVALGSEGEIPFYGKAIPIALLKGGRHLIFCHSKKKCDEIASKLRGMGLNAVAYYRGLDVSVIPTTGDVVVCATDALMTGFTGDFDSVIDCNVAVEQYVDFSLDPTFSIETRTAPQDAVSRSQRRGRTGRGRLGTYRYVASGERPSGMFDSVVLCECYDAGCSWYDLQPAETTVRLRAYLSTPGLPVCQDHLDFWESVFTGLTHIDAHFLSQTKQQGLNFSYLTAYQATVCARAQAPPPSWDEMWKCLVRLKPTLHGPTPLLYRLGPVQNETCLTHPITKYLMACMSADLEVTTSTWVLLGGVLAALAAYCLSVGCVVIVGHIELEGKPALVPDKEVLYQQYDEMEECSQAAPYIEQAQVIAHQFKEKILGLLQRATQQQAVIEPIVTTNWQKLEAFWHKHMWNFVSGIQYLAGLSTLPGNPAVASLMAFTASVTSPLTTNQTMFFNILGGWVATHLAGPQSSSAFVVSGLAGAAIGGIGLGRVLLDILAGYGAGVSGALVAFKIMGGECPTAEDMVNLLPAILSPGALVVGVICAAILRRHVGPGEGAVQWMNRLIAFASRGNHVSPTHYVPESDAAARVTALLSSLTVTSLLRRLHQWINEDYPSPCSDDWLRTIWDWVCSVLADFKAWLSAKIMPALPGLPFISCQKGYKGVWRGDGVMSTRCPCGAAITGHVKNGSMRLAGPRTCANMWHGTFPINEYTTGPSTPCPSPNYTRALWRVAANSYVEVRRVGDFHYITGATEDELKCPCQVPAAEFFTEVDGVRLHRYAPPCKPLLRDDITFMVGLHSYTIGSQLPCEPEPDVSVLTSMLRDPSHITAETAARRLARGSPPSEASSSASQLSAPSLKATCQTHRPHPDAELVDANLLWRQEMGSNITRVESETKVVVLDSFEPLRAETDDVEPSVAAECFKKPPKYPPALPIWARPDYNPPLLDRWKAPDYVPPTVHGCALPPRGAPPVPPPRRKRTIQLDGSNVSAALAALAEKSFPSSKPQEENSSSSGVDTQSSTTSKVPPSPGGESDSESCSSMPPLEGEPGDPDLSCDSWSTVSDSEEQSVVCCSMSYSWTGALITPCSAEEEKLPISPLSNSLLRHHNLVYSTSSRSASQRQKKVTFDRLQVLDDHYKTALKEVKERASRVKARMLTIEEACALVPPHSARSKFGYSAKDVRSLSSRAINQIRSVWEDLLEDTTTPIPTTIMAKNEVFCVDPAKGGRKPARLIVYPDLGVRVCEKRALYDVIQKLSIETMGPAYGFQYSPQQRVERLLKMWTSKKTPLGFSYDTRCFDSTVTEQDIRVEEEIYQCCNLEPEARKVISSLTERLYCGGPMFNSKGAQCGYRRCRASGVLPTSFGNTITCYIKATAAAKAANLRNPDFLVCGDDLVVVAESDGVDEDRAALRAFTEAMTRYSAPPGDAPQATYDLELITSCSSNVSVARDDKGRRYYYLTRDATTPLARAAWETARHTPVNSWLGNIIMYAPTIWVRMVMMTHFFSILQSQEILDRPLDFEMYGATYSVTPLDLPAIIERLHGLSAFTLHSYSPVELNRVAGTLRKLGCPPLRAWRHRARAVRAKLIAQGGKAKICGLYLFNWAVRTKTNLTPLPAAGQLDLSSWFTVGVGGNDIYHSVSRARTRHLLLCLLLLTVGVGIFLLPAR

SEQ ID NO: 114 - DNA, 3813 bp, synthetic construct
  source 1..3813: mol_type = other DNA; note = Omicron BA.2.75.2 spike protein with 2P modification nucleic acid sequence
atgtttgtgtttctggtcttactgcctctggtctcctctcagtgtgttaacttaataactaggactcaatcctacaccaattccttcacacgaggagtgtattatcccgataaagttttccgttcatcagttctccactctacccaagacctgtttctgcccttcttctcaaacgtaacctggttccacgctatccatgtttccggcacaaacggtacaaaacggttcgataatcccgtgttgccattcaacgacggtgtttattttgcttccaccgaaaagtccaacatcataaggggctggattttcggaaccactctggattctaagactcagtccctgctcattgtgaataacgctaccaacgtcgtaataaaagtgtgcgagttccaattctgtaatgatccctttctcgacgtctactatcacgaaaataataagtctaggatggaatccgaactgagagtatacagctccgctaataattgtacgtttgaatacgtctcccaaccgtttctcatggatctcgaaggcaaacaaggaaacttcaagaatctgcgggagttcgtcttcaagaatattgacgggtatttcaaaatttattccaaacatacaccagttaacctcggacgtgatcttccgcaaggattctccgccctggagccgcttgtcgacttaccaatcgggataaatataaccaggttccaaacactcttagcacttcatcgttcatatttaacacccggcgactcctcatctagttggactgctggggccgctgcatactacgtcgggtatctgcaaccacggacatttctgctcaagtataatgagaatggaacgattacagacgcagtcgattgtgccttggatcccctcagcgaaacaaaatgtactctcaaaagctttacagtagaaaaggggatttatcaaaccagtaacttcagagtccagccaacagaatccatagtgcgctttcctaatattaccaatctctgtcccttccatgaagtattcaatgctactacctttgcttccgtctacgcctggaataggaaaaggatatccaattgcgtggccgattatagcgttttgtacaactttgcacccttcttcgctttcaagtgttacggcgtcagtcctacaaaactgaatgacttatgtttcaccaatgtctacgccgattccttcgttattcgaggcaacgaagtcagtcaaatcgcacctggccaaacaggtaatatcgcagattacaattataaattaccagacgactttactgggtgcgtgattgcctggaatagcaacaaactcgattcaaaagtaagtggtaattacaactatctctacagattgtttcggaaaagtaagctcaagcctttcgaacgtgacataagtactgagatctaccaagcgggaaataaaccttgtaatggagttgcaggttccaactgttatttccccctgcaatcttatggctttcgtcccacatacggtgtaggccatcagccctatcgcgtcgtagtcctctcctttgaactgctccacgctcctgcaacagtgtgcggtcctaagaaatcaacaaacctggtaaagaataaatgtgtcaatttcaacttcaatgggctgactgggaccggagtattaacagaaagcaataagaagtttcttccgtttcagcaatttggaagggatatagccgacaccacggacgcagtaagagacccacaaacacttgagattttggatatcaccccatgttcattcggcggtgtctctgtgatcactcccggcacaaacacaagcaaccaagttgcagtactgtaccaaggcgtcaattgtaccgaagtacctgtcgcaatacacgccgaccaactcacgcctacttggcgggtctactccactggaagcaacgtgttccaaacacgggccggatgtttgatcggagccgagtatgtgaataattcctatgagtgcgacatccctataggagccggcatttgtgcgtcttaccaaacacaaaccaagagccatagacgagcaagaagtgttgctagtcagtccatcatagcctacacaatgtcccttggcgcagaaaatagtgtggcctattcaaataattccatcgctatcccaacgaactttaccataagcgtcactactgaaatactccctgtctccatgaccaagactagcgtggactgtaccatgtatatatgtggggacagcacagaatgctccaatctgttgttgcaatatggatccttttgcacccagctgaagagggccttgaccggcattgcggtggaacaagataagaatacacaggaagtctttgcccaagtgaaacaaatatataaaaccccaccgattaagtacttcggcggtttcaactttagccagattctccccgatccctcaaaacctagcaagcggtcatttattgaggatctgcttttcaataaagtgaccctggccgatgctggcttcatcaaacaatacggcgactgcctgggtgacatcgccgccagggatctcatttgcgcccagaagtttaacggattgactgtactgccgcccttgcttaccgacgagatgatagctcaatacaccagtgcgctccttgcagggaccatcacgagcggatggacatttggagctggggccgcgcttcaaattccttttgcaatgcagatggcttacaggttcaacggaattggggtgacccaaaacgtactctatgagaatcaaaagctcattgctaaccagttcaattcagccattgggaaaattcaagacagcttaagttccactgcaagcgcgttggggaaactgcaagatgttgtgaaccacaatgcgcaggcgctcaacactctggtgaagcagctcagctcaaagtttggcgctataagtagtgttttgaatgacatcctgtctcggctggacccacccgaggccgaagtgcaaattgatcgtctgatcacaggaaggcttcaaagcctgcagacgtatgttacacaacaactgattcgggccgcagaaataagggcatctgccaacttggccgctaccaaaatgtctgagtgtgttttgggacaatccaaacgggtcgatttctgcggtaaaggatatcatctcatgtctttccctcagagtgcccctcacggcgtagtcttcttacacgtaacctacgtacccgcacaagagaagaatttcactaccgcaccagcaatctgccatgacgggaaagcccactttccgagggaaggagtattcgtatctaacggcacgcactggttcgtgacccaaagaaatttctatgagccacagatcataactacagacaatactttcgttagtggtaactgtgacgttgtgataggaatagtcaataacaccgtgtacgatccccttcaacctgagctcgactccttcaaggaagagctcgataagtattttaagaatcacactagcccggatgtggacttgggcgacatcagcgggattaacgcaagcgtagtgaacatccagaaagagattgacaggctcaatgaagtggccaagaatctcaacgaatctctgataaatctccaggaactgggaaagtatgagcaatacataaagtggccatggtatatctggctgggatttatcgcagggctgatagctattgttatggtcaccattatgttgtgttgtatgacgtcatgctgctcatgtctgaaaggttgctgtagttgcgggagttgctgcaaatttgacgaagacgattcagagccagtcctcaagggcgttaagcttcactacacttag

SEQ ID NO: 115 - protein, 1270 aa, synthetic construct
  source 1..1270: note = Omicron BA.2.75.2 spike protein with 2P modification amino acid sequence
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGSNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLINLQELGKYEQYIKWPWYIWLGFIAGLIAIVMVTIMLCCMTSCCSCLKGCCSCGSCCKFDEDDSEPVLKGVKLHYT

SEQ ID NO: 116 - DNA, 3807 bp, synthetic construct
  source 1..3807: mol_type = other DNA; note = Omicron-22E (BQ.1) spike protein with 2P modification nucleic acid sequence
atgtttgtatttctcgttctgctccctttagtgtctagccaatgcgtaaacctgatcactcgcacccaatcttataccaattcttttactagaggagtgtactatcccgataaggttttccgcagctccgtgctccatagtacgcaggatcttttcctgccattcttcagtaacgtcacatggtttcatgctatttcagggacaaacgggacaaagagattcgacaatcccgtgctgcccttcaatgacggtgtatattttgccagcacggagaagagtaacataataaggggatggatctttggtactacattggactcaaagacacaaagcctgttgattgtgaataacgccaccaacgtggtgatcaaggtctgtgaatttcaattctgtaatgacccattcctcgacgtatattaccataagaacaacaagagttggatggagagtgaatttcgagtctatagtagtgcaaataattgcactttcgaatacgttagtcaacctttcctgatggatctggaagggaaacaaggcaacttcaagaatttaagagagttcgtcttcaagaatatagacggatatttcaaaatctattccaagcatactccaatcaatctgggcagggatctgccccagggcttcagcgcactggagcccctcgtcgatttgcctataggcatcaatataacaaggtttcaaacccttctggccttacatcgaagctatctcacacccggcgacagcagtagtggatggacagcaggagctgctgcatattacgtgggatatcttcaaccgagaaccttcttattaaaatacaatgagaacgggactataacagatgcagttgattgtgcactcgaccccctcagcgagacaaagtgtacactgaaatcattcacagtcgagaaaggcatctaccagacaagcaatttccgagtgcagcccactgaatctatagtgaggttcccaaatatcactaatttgtgcccatttgacgaagtattcaatgctaccacttttgcctctgtgtatgcatggaatcgcaagagaatttccaattgcgtggctgattactcagtgttgtacaactttgctccattctttgctttcaagtgctatggagtatccccaaccaaattaaacgatctttgttttaccaatgtctatgctgattcattcgtaattcgagggaatgaggttagtcaaattgctccaggacaaactggcaacattgccgattataactataaactcccagacgattttaccgggtgcgtgatcgcttggaactccaataagttggacagtactgtcggcggaaactataattatagatatcgcctcttcaggaaaagtaagcttaaaccctttgaacgcgatatctccaccgagatttatcaagcaggtaataaaccatgcaacggtgtagctggagttaactgttatttccctctgcagtcatacggttttcgaccaacttacggggtgggccatcaaccctaccgagttgtagtgcttagcttcgaactgctccatgctccggccaccgtctgtggccccaagaaatctactaatcttgtaaagaataagtgtgtgaacttcaattttaacggtcttaccggcacaggtgtgctcacagagagtaacaagaaattccttccatttcaacaattcgggagagacattgctgataccaccgatgcagtgcgcgacccccaaacgttagagatacttgatataaccccatgttcattcggtggagtatcagtgatcactcctggtactaatacttctaaccaagttgctgtcttataccagggcgtaaattgtacagaagtgccggtcgcgatccatgcggaccaactcacccctacatggcgcgtgtactccacggggagtaacgtgtttcaaaccagggcaggctgcctcattggtgctgagtatgtgaacaactcctacgagtgcgatatccctataggtgccgggatttgcgcctcatatcagacccaaactaaaagtcatcgcagggccaggtctgtagcatctcaaagtataattgcatatacaatgtcacttggcgctgagaatagcgttgcgtattctaataatagcattgccatacctaccaatttcactatctccgtgacaacagagattctgccagtctctatgaccaaaacatcagtcgactgcacgatgtatatctgcggggatagtaccgagtgctctaatctgctgctccaatatggaagtttctgtactcagcttaaaagggctcttactggtatcgccgtagagcaagataagaatacgcaggaagtctttgcgcaagttaaacaaatttacaagacaccgccgataaaatattttggcggtttcaacttttcccagatcctgcccgatccctcaaaaccaagcaaacgctctttcatcgaagacctgttgtttaataaagtgaccttggccgatgctgggtttatcaaacaatacggtgactgccttggggacatagcagctagggacttaatctgcgcccagaagttcaatggactgactgttctcccaccgttgttgacagacgaaatgatagcccaatacacgtccgctctcctggctggaacaataacctcagggtggaccttcggggctggcgctgctctgcaaattcctttcgcaatgcaaatggcttaccgttttaatggcatcggcgttacccagaacgtactgtacgagaatcaaaagctgattgcaaatcaattcaatagtgccatcggcaagattcaagatagcctgtcctctacggcaagcgctttgggtaaacttcaagacgtggtgaatcataatgctcaagcccttaacacactggtgaagcaactctccagcaagtttggcgcaattagctctgtacttaatgatattctgagccggctggacccaccagaggccgaagtccaaatagaccgtctgattacgggacggcttcaatccttgcagacctacgttacccaacaacttatcagggctgccgagataagagcaagtgccaaccttgctgccaccaagatgtccgagtgtgtactcggccaatccaagcgcgtcgacttttgtgggaagggatatcacttgatgtctttccctcaatctgcccctcacggcgtggtgttcctgcatgtgacctacgtgccagcgcaagaaaagaactttaccactgctccagcaatttgtcacgacggcaaagcacattttcccagggaaggagtgtttgtttctaatggaacgcactggttcgtaacacaaagaaatttctatgaaccccaaatcattactactgataacaccttcgtttctggtaactgtgacgtagttatcggaatcgtaaacaacactgtatatgaccctctgcagccagagctcgacagttttaaggaagagttggataagtattttaagaatcatacctctcctgacgtcgacctcggcgacatttccggcataaatgcctcagtcgtgaatatccagaaagaaatagaccgcttgaacgaggttgcgaagaatttgaacgagtccttgatagacctgcaagagctgggaaagtatgagcaatacatcaagtggccgtggtatatatggctcgggtttattgctggtttaatcgctatcgtgatggtgacaataatgctttgttgtatgacatcatgttgcagttgtctcaaagggtgttgcagttgcggctcctgctgcaagttcgatgaggacgattctgaaccagtgctcaaaggggttaagcttcattatacgtag

SEQ ID NO: 117 - protein, 1268 aa, synthetic construct
  source 1..1268: note = Omicron-22E (BQ.1) spike protein with 2P modification amino acid sequence
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 118 - DNA, 3804 bp, synthetic construct
  source 1..3804: mol_type = other DNA; note = Omicron-22E (BQ.1) spike protein with 2P modification and furin cleavage mutation nucleic acid sequence
cgtataatgaagcttaacccctttgagcactggttcagaatcgtcctcatcgaacttgcagcaggagccgcaactgcaacaccctttgagacaactgcaacatgatgtcatacaacaaagcattattgtcaccatcacgatagcgattaaaccagcaataaacccgagccatatataccacggccacttgatgtattgctcatactttcccagctcttgcaggtctatcaaggactcgttcaaattcttcgcaacctcgttcaagcggtctatttctttctggatattcacgactgaggcatttatgccggaaatgtcgccgaggtcgacgtcaggagaggtatgattcttaaaatacttatccaactcttccttaaaactgtcgagctctggctgcagagggtcatatacagtgttgtttacgattccgataactacgtcacagttaccagaaacgaaggtgttatcagtagtaatgatttggggttcatagaaatttctttgtgttacgaaccagtgcgttccattagaaacaaacactccttccctgggaaaatgtgctttgccgtcgtgacaaattgctggagcagtggtaaagttcttttcttgcgctggcacgtaggtcacatgcaggaacaccacgccgtgaggggcagattgagggaaagacatcaagtgatatcccttcccacaaaagtcgacgcgcttggattggccgagtacacactcggacatcttggtggcagcaaggttggcacttgctcttatctcggcagccctgataagttgttgggtaacgtaggtctgcaaggattgaagccgtcccgtaatcagacggtctatttggacttcggcctctggtgggtccagccggctcagaatatcattaagtacagagctaattgcgccaaacttgctggagagttgcttcaccagtgtgttaagggcttgagcattatgattcaccacgtcttgaagtttacccaaagcgcttgccgtagaggacaggctatcttgaatcttgccgatggcactattgaattgatttgcaatcagcttttgattctcgtacagtacgttctgggtaacgccgatgccattaaaacggtaagccatttgcattgcgaaaggaatttgcagagcagcgccagccccgaaggtccaccctgaggttattgttccagccaggagagcggacgtgtattgggctatcatttcgtctgtcaacaacggtgggagaacagtcagtccattgaacttctgggcgcagattaagtccctagctgctatgtccccaaggcagtcaccgtattgtttgataaacccagcatcggccaaggtcactttattaaacaacaggtcttcgatgaaagagcgtttgcttggttttgagggatcgggcaggatctgggaaaagttgaaaccgccaaaatattttatcggcggtgtcttgtaaatttgtttaacttgcgcaaagacttcctgcgtattcttatcttgctctacggcgataccagtaagagcccttttaagctgagtacagaaacttccatattggagcagcagattagagcactcggtactatccccgcagatatacatcgtgcagtcgactgatgttttggtcatagagactggcagaatctctgttgtcacggagatagtgaaattggtaggtatggcaatgctattattagaatacgcaacgctattctcagcgccaagtgacattgtatatgcaattatactttgagatgctacagagctggcgctcccatgacttttagtttgggtctgatatgaggcgcaaatcccggcacctatagggatatcgcactcgtaggagttgttcacatactcagcaccaatgaggcagcctgccctggtttgaaacacgttactccccgtggagtacacgcgccatgtaggggtgagttggtccgcatggatcgcgaccggcacttctgtacaatttacgccctggtataagacagcaacttggttagaagtattagtaccaggagtgatcactgatactccaccgaatgaacatggggttatatcaagtatctctaacgtttgggggtcgcgcactgcatcggtggtatcagcaatgtctctcccgaattgttgaaatggaaggaatttcttgttactctctgtgagcacacctgtgccggtaagaccgttaaaattgaagttcacacacttattctttacaagattagtagatttcttggggccacagacggtggccggagcatggagcagttcgaagctaagcactacaactcggtagggttgatggcccaccccgtaagttggtcgaaaaccgtatgactgcagagggaaataacagttaactccagctacaccgttgcatggtttattacctgcttgataaatctcggtggagatatcgcgttcaaagggtttaagcttacttttcctgaagaggcgatatctataattatagtttccgccgacagtactgtccaacttattggagttccaagcgatcacgcacccggtaaaatcgtctgggagtttatagttataatcggcaatgttgccagtttgtcctggagcaatttgactaacctcattccctcgaattacgaatgaatcagcatagacattggtaaaacaaagatcgtttaatttggttggggatactccatagcacttgaaagcaaagaatggagcaaagttgtacaacactgagtaatcagccacgcaattggaaattctcttgcgattccatgcatacacagaggcaaaagtggtagcattgaatacttcgtcaaatgggcacaaattagtgatatttgggaacctcactatagattcagtgggctgcactcggaaattgcttgtctggtagatgcctttctcgactgtgaatgatttcagtgtacactttgtctcgctgagggggtcgagtgcacaatcaactgcatctgttatagtcccgttctcattgtattttaataagaaggttctcggttgaagatatcccacgtaatatgcagcagctcctgctgtccatccactactgctgtcgccgggtgtgagatagcttcgatgtaaggccagaagggtttgaaaccttgttatattgatgcctataggcaaatcgacgaggggctccagtgcgctgaagccctggggcagatccctgcccagattgattggagtatgcttggaatagattttgaaatatccgtctatattcttgaagacgaactctcttaaattcttgaagttgccttgtttcccttccagatccatcaggaaaggttgactaacgtattcgaaagtgcaattatttgcactactatagactcgaaattcactctccatccaactcttgttgttcttatggtaatatacgtcgaggaatgggtcattacagaattgaaattcacagaccttgatcaccacgttggtggcgttattcacaatcaacaggctttgtgtctttgagtccaatgtagtaccaaagatccatccccttattatgttactcttctccgtgctggcaaaatatacaccgtcattgaagggcagcacgggattgtcgaatctctttgtcccgtttgtccctgaaatagcatgaaaccatgtgacgttactgaagaatggcaggaaaagatcctgcgtactatggagcacggagctgcggaaaaccttatcgggatagtacactcctctagtaaaagaattggtataagattgggtgcgagtgatcaggtttacgcattggctagacactaaagggagcagaacgagaaatacaaacat

SEQ ID NO: 119 - protein, 1268 aa, synthetic construct
  source 1..1268: note = Omicron-22E (BQ.1) spike protein with 2P modification and furin cleavage mutation amino acid sequence
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 120 - DNA, 3807 bp, synthetic construct
  source 1..3807: mol_type = other DNA; note = Omicron BA.5 spike protein with 2P modification nucleic acid sequence
atgttcggttttctggtcctcttaccccttgtgtcctcacaatgcgtcaacctgataaccaggacacaaagttataccaactcctttactcgtggtgtctactaccctgataaagtatttagaagcagcgtgctgcatagcacacaggatctgttcctgcccttcttctccaacgtcacatggttccacgcaatcagtggcacgaacggaaccaagagatttgataatccggttctccccttcaacgacggcgtctattttgcttctacagagaaaagcaacattattcgaggttggattttcgggaccactcttgattccaaaacacaaagtctcctcatcgtaaataacgccacgaatgtggtcataaaagtttgcgaatttcagttttgcaacgatcctttcctggatgtctactatcacaagaacaacaaaagttggatggaatccgaatttcgagtatattcctcagcaaacaactgcacttttgagtatgtgtctcaaccattcctgatggatctcgaaggcaaacaaggaaactttaagaacctgcgcgagtttgtctttaagaacatagacgggtatttcaaaatttattctaaacatacgccgattaacttggggcgcgatctcccgcaagggttttccgccttagagcccctcgtcgatctgcctattggaatcaacatcacacggtttcagactttacttgcactgcacagaagttatctcacacccggagactcctcatcaggatggactgcgggtgctgctgcttattacgtcgggtatctgcaaccaagaacatttcttctgaaatataatgagaatggcactattacggatgccgtggattgcgcacttgaccccctctccgaaacaaaatgtactctgaagagttttacagtggaaaagggaatttatcaaacaagcaatttccgggtgcaaccgacagaaagtatcgtgaggtttccaaacattacaaatctttgcccattcgacgaggtattcaacgctactcggtttgcttctgtttatgcatggaaccggaagaggatctccaattgtgtagcagattacagcgtgctttacaacttcgctccattcttcgcatttaaatgttatggcgtttctcctactaaacttaatgatctgtgcttcacaaatgtgtatgccgacagcttcgttattaggggtaatgaggtatcccagattgctcccggacaaacagggaacatcgcggactataactacaagttgcctgatgattttactggctgcgtaattgcctggaactctaataagcttgactccaaagtaggcgggaattataactatcgatatagactgtttcgcaagtctaaccttaaaccctttgagcgggatatctcaacagaaatttatcaagccggaaataagccctgcaatggggttgcaggagttaattgttatttccccctgcaaagttacgggttccgccccacatatggggtcggtcaccaaccataccgggtcgtcgttctgtcattcgaattgttacatgcacccgctaccgtatgcggccctaagaagtcaactaatcttgtcaagaataaatgcgtaaattttaactttaatggattgactgggacaggtgtgttgaccgagtcaaacaagaagtttctgccctttcaacaatttggacgggacatagccgatacgacagatgcggttcgggacccgcaaacactggaaatactggatataactccgtgtagttttggcggtgttagcgtaattacacccggtacaaatacaagcaaccaagtagcagtcctgtatcaaggcgttaactgtactgaggtaccagtggcaattcacgcagatcagctcacacccacctggcgagtgtactctaccggatctaatgtgttccaaactcgtgccggatgcctgataggagctgagtacgtgaataatagttatgagtgcgatatacccattggggccggcatatgcgcatcctaccaaacccaaaccaaatctcataggcgtgctagaagtgtggccagccaatctatcatcgcttatacaatgtcccttggcgcagaaaattccgtggcttactcaaacaactccatagccattcccactaattttacgattagtgttactactgagatcctgccggtgtctatgactaaaacctctgtagattgtacgatgtacatctgtggggactccactgaatgctccaatctgctcctgcaatacgggagcttctgtacccaactgaaacgagcactgactggcattgccgtggaacaagacaagaacacacaagaagtattcgcacaagtaaaacaaatttataagacacccccaatcaaatatttcggtggtttcaactttagccagatcttacccgatccaagcaaaccctcaaaacggagctttattgaggacttactctttaataaagttaccttggcggacgctggatttattaaacagtacggggattgtctgggtgatattgcagcacgcgatttaatttgtgcccaaaagttcaatggtcttaccgttctgccgcctctgttaacagacgagatgatcgcccaatatacctctgccctgctggcaggcactataacttccggttggacgtttggcgctggggccgctctccagatcccatttgctatgcaaatggcttatcggttcaacggcattggggttacacagaacgtgttgtatgagaaccagaaattaattgccaaccagtttaattctgccatcggtaagatacaagattcattgtcttctactgctagtgcactcggaaagttgcaagacgtggtcaatcacaatgcacaggccttaaatactctggttaaacaactcagctctaaattcggtgccatttcatctgtcctgaatgatattttgtcaagactcgacaaggtagaggcggaagtacaaattgataggctcatcacagggcgactccagtcattgcagacatacgtgacacaacagctgattcgcgcagcggaaataagggcctcagcaaatctggctgcaaccaagatgtcagaatgcgttttaggtcaaagtaaacgggttgatttctgtggtaagggataccatctcatgtcattcccacaaagcgcccctcacggcgttgttttcttgcatgtcacatatgtgccagcccaagaaaagaattttacaactgcccccgccatttgtcacgatggtaaagctcattttccacgcgagggtgttttcgtgtcaaatggaacccattggttcgtcactcaacgcaatttctatgaaccccaaatcataaccactgacaatacgtttgtttcaggcaattgcgacgtggtcataggaattgttaacaatactgtttatgatccgctgcaacccgagttggattcctttaaggaagagttggacaagtattttaagaaccacaccagcccggatgttgatctgggagatatttcaggaatcaatgcaagcgtggttaatatccaaaaggagatcgatcgtctcaatgaagtggccaagaatttgaatgaaagtttaattgaccttcaagaactcgggaagtacgaacaatatattaaatggccctggtacatatggcttggcttcatcgccggcctcattgccatcgtgatggttacaataatgttatgttgcatgacatcttgctgctcctgtctcaagggatgttgctcttgtggtagctgttgtaaattcgatgaggatgattctgaaccagtgcttaagggagtcaaacttcactatacatga

SEQ ID NO: 121 - protein, 1268 aa, synthetic construct
  source 1..1268: note = Omicron BA.5 spike protein with 2P modification amino acid sequence
MFGFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 122 - DNA, 1251 bp, synthetic construct
  source 1..1251: mol_type = other DNA; note = Omicron BQ.1 Nucleocapsid nucleic acid sequence
atgagcgacaacggcccccaaaaccagaggaacgcactgcggataacattcggtggtccctcagattctacaggaagtaaccaaaacggcggagctcggagtaagcaaagaaggccgcaaggcctgcctaacaacactgcgtcttggttcactgccctgactcaacacggcaaagagggactgaaattccccagaggtcaaggggtgccaatcaacactaacagctcccctgatgaccaaattggctattaccggagagctacgcgacggatcaggggcggcgacggcaaaatgaaggaccttagcccccgctggtacttctactatttaggtaccggacccgaagccggccttccctacggcgctaataaagacggcattatatgggtggccactgatggagcattgaatacacctaaagatcatataggaacccgcaatccggccaataacgccgctattgtgttacaattgccccagggtacaacccttcctaaaggcttctacgccgaggggagccggggcggaagccaagcctcttcccggagtagctctcggagcaggaactcctcaaggaactccactcctgggagcagcaaaagaacaagccctgctcgtatggccgggaatggtggggatgctgcattggccttgttactcctggataggctgaatcaacttgaatccaagatgtcagggaaaggtcaacaacaacaaggacaaaccgtaactaagaagagcgccgccgaagccagcaagaaacccagacaaaagcgcactgctactaaggcttataatgtcacccaagcctttgggcgccgcggacctgagcagacccaggggaacttcggggatcaggagctgatcaggcaaggcactgattataagcactggcctcagatagcgcaattcgccccatcagcttccgcattctttggcatgtctcgcatcggcatggaagttactccttctggaacatggttgacgtacaccggggcaatcaaactcgatgataaagaccccaactttaaggaccaagttatcttgttgaacaaacacattgacgcttataaaacattcccacccaccgaacctaagaaggataagaagaataaggcagatgagacccaggctctcccccaaaggcaaaagaagcaacaaactgtaaccctgctgcctgccgcagatttagacgatttcagcaagcaactccaacaaagcatgagcagagctgattctacccaagcctga

SEQ ID NO: 123 - protein, 416 aa, synthetic construct
  source 1..416: note = Omicron BQ.1 Nucleocapsid amino acid sequence
MSDNGPQNQRNALRITFGGPSDSTGSNQNGGARSKQRRPQGLPNNTASWFTALTQHGKEGLKFPRGQGVPINTNSSPDDQIGYYRRATRRIRGGDGKMKDLSPRWYFYYLGTGPEAGLPYGANKDGIIWVATDGALNTPKDHIGTRNPANNAAIVLQLPQGTTLPKGFYAEGSRGGSQASSRSSSRSRNSSRNSTPGSSKRTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKNKADETQALPQRQKKQQTVTLLPAADLDDFSKQLQQSMSRADSTQA

SEQ ID NO: 124 - DNA, 3810 bp, synthetic construct
  source 1..3810: mol_type = other DNA; note = Omicron XBB1.5 spike protein with 2P modification nucleic acid sequence
atgtttgtgttcctcgtactgcttcctcttgtatcctcacaatgcgtgaacctgataacgcgtacacaatcctataccaactcatttacacggggagtctactaccccgacaaagtgtttaggtcctctgttttgcatagcacgcaagatctgtttctcccattcttttcaaatgtaacatggttccacgccatacacgtgtccggaaccaatggcaccaagcgcttcgacaatccagctctcccttttaacgatggtgtatatttcgcatcaactgagaagagcaatataatacgcggctggattttcggaacaactttagactcaaagacgcaatctctcctcattgttaataacgccaccaacgtggtcatcaaagtctgtgaatttcaattctgtaatgatccgttcttggatgtctaccaaaagaacaataaatcctggatggaatctgagtttcgggtgtattcctcagctaacaattgtactttcgagtacgtgtctcagcctttcttcatggacctcgagggtaaagaaggcaatttcaagaaccttcgcgagttcgtcttcaagaatattgacgggtattttaagatttattctaaacacacacctattaatcttgagcgggatctcccccaaggcttcagtgcactggagccacttgtcgacttgccgataggcatcaatattacaaggtttcaaactttactggccttgcatcggtcatacctgacacctgtcgattcctcatccggctggacggctggcgcggctgcctactatgtcggctacttacaacctcgcacatttctgctcaaatataacgaaaacggtacaattaccgatgccgtagactgtgccttagaccccctctcagaaactaagtgcacattgaaatccttcacagtggaaaaggggatttatcagacctcaaattttcgggttcagccaaccgaaagcattgtccggttcccaaacattactaatctttgtcccttccacgaagtcttcaacgcaaccaccttcgcaagcgtctacgcatggaatagaaaacgaatttcaaattgtgtcgccgattatagcgttatctataattttgcaccgttctttgcatttaagtgctatggcgtgagcccaacaaagttaaacgacctgtgtttcacaaatgtatacgctgactcattcgtgataaggggcaacgaggttagccaaatcgccccgggacaaacaggtaacatagctgactataactacaaactcccagacgacttcacgggttgtgtgatcgcatggaactctaacaaactcgatagtaagccctctggaaattataattacctttatcgcttattccgtaaatccaaactgaaaccttttgaacgggacatcagtactgaaatttaccaggcaggcaacaagccttgtaacggggtggccggtcccaattgttactctcccctgcaaagctatggtttccgaccgacgtacggcgtcgggcatcagccctaccgcgtggtcgtcctgtcctttgagcttctgcacgcaccagccacagtttgcggccctaagaagtccacaaacctggtaaagaacaaatgtgttaacttcaactttaacgggctcaccggaactggagtattgactgagtccaataagaagttcttaccctttcaacaattcggccgagacatcgcagacacaactgacgccgttcgggatccacaaaccttggaaattttagatattaccccctgtagttttggtggggtgtccgttattacacccggcactaacacgtctaaccaggtcgccgtgctgtatcagggcgtcaattgtactgaggtacccgttgcaattcatgccgatcagctgacaccaacttggagagtatacagtaccggcagcaatgtgtttcagactcgcgccggctgcctgataggagctgaatacgtgaacaatagttatgagtgtgatatccccattggtgctgggatctgtgcctcataccagacgcaaacaaagagccatcgtcgcgcacgttccgttgcaagccaatctataatcgcatataccatgtccctcggagcagaaaactccgtggcatattcaaacaatagtatcgcgattccgacaaattttacaatatccgtcacgaccgagattttgccagtcagcatgactaaaacatctgtcgattgtactatgtatatatgtggtgatagtacagaatgcagtaatctgttgctgcagtatggaagcttttgcacccaactcaagcgggctcttactggaattgccgttgaacaagataagaatacccaggaagtgtttgctcaggtcaaacagatctataagactccaccaataaaatactttggtggttttaatttctcccaaatactccccgatcccagcaagccgtctaagaggtcttttatcgaagatcttcttttcaacaaggtcacacttgctgatgctggatttattaaacaatatggcgattgtttgggagacattgcggcgcgggaccttatatgtgcgcaaaagttcaatggtctcaccgtccttccacccttattaacagatgagatgatcgcccaatacactagcgcattactggctggcactatcacctctgggtggacattcggagcaggcgctgctctgcaaatcccatttgcaatgcagatggcatacagatttaatggcattggggtcacgcaaaacgttctttatgaaaaccaaaagttgattgccaaccagttcaattccgcgatcgggaagatccaagattcactgtcaagtaccgcttccgcactcggtaaactccaagatgtggtcaaccataatgcacaagctcttaataccctggtaaaacaactctcttcaaagttcggcgcaatcagttccgtgctcaatgatattctttccaggctcgacccgccagaggctgaggtccaaatcgacagattaataacaggccgtttacaatcactccaaacttacgtgacacaacaattaattagggcagcggaaatccgcgcttctgccaacctggccgcaacaaagatgtccgaatgtgtcctggggcaatccaaaagagttgacttttgtggcaagggttaccacttaatgtcctttccccaaagcgctcctcatggcgtggttttcctgcacgtgacatatgtgcctgcacaagagaagaactttactactgcacctgccatatgccatgatggcaaggctcactttccccgcgagggcgtgttcgtttccaacgggacccactggttcgtcacccaacggaacttttatgagccacaaataattacaaccgataatacctttgtatcaggtaactgcgatgtggtcattggaatagtgaacaataccgtatatgaccctctccaacctgagctcgattccttcaaagaggaattagacaaatactttaagaatcatacttcccccgatgtcgacctcggcgacatcagtggtatcaacgccagtgttgtcaatatccaaaaggaaatcgacagactcaatgaggtagcaaagaatctcaacgaaagtcttatagatctgcaagaactggggaagtatgaacaatatatcaagtggccctggtatatctggcttgggtttatcgctggactcatcgccattgtgatggttacaattatgctctgttgtatgactagctgttgcagctgtcttaaaggatgctgcagctgtggatcttgctgtaagttcgatgaggatgactcagagcctgtattgaagggcgtgaaactccactacacctga

SEQ ID NO: 125 - protein, 1269 aa, synthetic construct
  source 1..1269: note = Omicron XBB1.5 spike protein with 2P modification amino acid sequence
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFFMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 126 - DNA, 3813 bp, synthetic construct
  source 1..3813: mol_type = other DNA; note = Beta spike protein with 2P modification and furin cleavage mutation nucleic acid sequence
atgttcgttttcctcgtgctgctccccctcgtctcttcacaatgcgtgaatcttacgacaagaacacaattaccgcctgcctacacaaattcatttacaaggggcgtatactatcccgataaggtgtttcgatcttccgtgctgcattcaacccaagacctctttcttcccttcttctccaacgtgacttggttccacgccatacatgttagcggcacaaatggtactaaacgcttcgccaaccccgttctcccattcaatgatggagtgtatttcgcaagcaccgaaaagtctaacattattcgaggctggatatttggaactactctggactccaagacacaatctttgctcattgttaacaacgctactaatgtggtgattaaagtctgtgagttccaattttgtaacgatccatttctgggagtctattatcacaagaacaacaaatcctggatggagagtgaatttcgggtatacagctccgcaaacaactgtacgttcgaatacgtatcccagccgtttctcatggacttggaagggaaacaagggaactttaagaatctccgtgaatttgtgttcaagaacatcgacggttatttcaaaatatattctaaacacacgcccataaaccttgtccgaggactcccccagggattttccgctctcgaacccttggttgacctgcctataggcattaacatcactagattccaaactctccacagatcatacttaacgcctggagatagctcatctggatggacagccggggcagcagcatattatgtcgggtacctgcaaccgagaactttcctcttaaaatacaacgaaaatggcaccattactgacgccgtggattgtgcccttgaccctttgagcgaaaccaagtgtactttaaaatccttcactgtagagaaaggcatataccaaacttccaattttagggtgcaaccaactgaatcaattgtcagattccccaatattacgaacctgtgtccgtttggcgaagtgtttaacgccacgcgcttcgcgtctgtctatgcctggaatcggaagagaataagcaattgtgtcgcagactacagtgtcttgtacaatagcgcttccttcagtaccttcaaatgttatggggtgtctcctacgaaactcaacgatttatgcttcaccaacgtctacgctgactcctttgtgataaggggcgatgaagtacgacaaatagcgccaggacaaaccggcaacatcgctgactacaactataagctgccagatgattttacagggtgtgtcatcgcttggaactctaataacttggactcaaaagttggtggcaattacaactatctctacaggttatttcgcaaaagcaacctcaaaccatttgagagagatatctctaccgaaatctaccaggccggcagcaccccttgtaacggtgtgaagggtttcaattgttattttcctctccaatcctatggctttcaaccaacttatggtgtcggttaccaaccatatagagtagttgtgttgtcctttgagttactccatgcccctgccactgtgtgtggacctaagaaatctaccaacttagttaagaacaaatgcgtaaactttaactttaacggattaaccggaaccggggtgttgactgaaagtaacaagaaattcttgcctttccaacaatttggcagagacatcgcagataccacagatgccgttcgtgatccccaaacactcgaaatcttggacataacaccctgttcttttggtggagtaagcgttataactccaggcactaacacttccaaccaagtcgcagtactctatcaaggggttaactgcacagaggtaccagtggccatccatgcagaccaactcacccccacatggagagtttacagcacggggtccaatgtctttcaaacgcgagcgggctgtctgataggagcagaacacgtcaataactcatacgagtgtgatatccccattggagcagggatttgcgcctcttatcaaacgcaaaccaactcacctggttccgcatccagcgtcgcgagccaatcaattatagcctatacaatgtccttaggcgttgaaaacagcgtcgcttactcaaataacagcatagccatacccacaaacttcacaattagcgttaccactgaaatactcccagttagcatgacaaagacttccgttgattgcaccatgtacatctgtggcgactcaacagagtgcagcaatctgctgctccaatacgggtctttctgtacccagctcaatagggcactgactggtatagcagtggaacaagacaagaatactcaggaagtattcgcccaggtgaaacagatttacaaaactccccctatcaaagatttcggcggctttaacttctcccaaatcctgcccgatccttccaaaccgtccaaaaggtcctttattgaagacctgctgtttaacaaagtgacccttgcagatgccggattcattaaacaatatggggactgtctcggggatatagccgcgagggatttaatctgtgctcaaaagttcaatggcctcaccgttctcccaccccttcttaccgacgaaatgattgcccaatatactagtgcgctcctggctggtacgataactagcggctggactttcggcgccggtgcggccctgcaaataccctttgctatgcaaatggcgtaccgctttaatggcatcggggttacacaaaatgtactttacgagaatcaaaagctcatcgcaaaccaatttaattcagcaatcggaaagatccaagactccttgagcagcaccgcgtctgccttgggcaaactccaggacgttgtgaaccaaaatgcgcaggcactgaacactcttgttaaacaattgagtagcaactttggcgcaattagctctgtacttaatgatattctgagccggctggacccaccagaggccgaagtccaaatagaccgtctgattacgggacggcttcaatccttgcagacctacgttacccaacaacttatcagggctgccgagataagagcaagtgccaaccttgctgccaccaagatgtccgagtgtgtactcggccaatccaagcgcgtcgacttttgtgggaagggatatcacttgatgtctttccctcaatctgcccctcacggcgtggtgttcctgcatgtgacctacgtgccagcgcaagaaaagaactttaccactgctccagcaatttgtcacgacggcaaagcacattttcccagggaaggagtgtttgtttctaatggaacgcactggttcgtaacacaaagaaatttctatgaaccccaaatcattactactgataacaccttcgtttctggtaactgtgacgtagttatcggaatcgtaaacaacactgtatatgaccctctgcagccagagctcgacagttttaaggaagagttggataagtattttaagaatcatacctctcctgacgtcgacctcggcgacatttccggcataaatgcctcagtcgtgaatatccagaaagaaatagaccgcttgaacgaggttgcgaagaatttgaacgagtccttgatagacctgcaagagctgggaaagtatgagcaatacatcaagtggccgtggtatatatggctcgggtttattgctggtttaatcgctatcgtgatggtgacaataatgctttgttgtatgacatcatgttgcagttgtctcaaagggtgttgcagttgcggctcctgctgcaagttcgatgaggacgattctgaaccagtgctcaaaggggttaagcttcattatacgtag

SEQ ID NO: 127 - protein, 1270 aa, synthetic construct
  source 1..1270: note = Beta spike protein with 2P modification and furin cleavage mutation amino acid sequence
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 128 - DNA, 824 bp, synthetic construct
  source 1..824: mol_type = other DNA; note = CMV intron A
gtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgtgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcag

SEQ ID NO: 129 - DNA, 54 bp, synthetic construct
  source 1..54: mol_type = other DNA; note = leader sequence nucleic acid sequence
atggattggacctggatcctgttcctggttgcagcagctacccgtgtgcactct

SEQ ID NO: 130 - protein, 18 aa, synthetic construct
  source 1..18: note = leader sequence amino acid sequence
MDWTWILFLVAAATRVHS

SEQ ID NO: 131 - DNA, 418 bp, synthetic construct
  source 1..418: mol_type = other DNA; note = SV40 promoter
gcgcagcaccatggcctgaaataacctctaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 132 - DNA, 668 bp, synthetic construct
  source 1..668: mol_type = other DNA; note = SARS-CoV-2 M Protein
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgcttcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

(19) United States
(12) Patent Application Publication
(10) Pub. No.: US 2025/0339510 A1
(43) Pub. Date: Nov. 6, 2025

(54) POLYNUCLEOTIDE VACCINE FORMULATIONS AND METHODS OF USING THE SAME

(71) Applicant: IMUNON, INC., Lawrenceville, NJ (US)

(72) Inventors: Carlo IAVARONE, Lawrenceville, NJ (US); Majed Mohd MATAR, Madison, AL (US); Subeena SOOD, Lawrenceville, NJ (US); John HENDERSON, Lawrenceville, NJ (US); Jessica KIM, Lawrenceville, NJ (US); Brian Jeffrey SPARKS, Huntsville, AL (US); Michael Henry TARDUGNO, Princeton, NJ (US); Jean D. BOYER, Lawrenceville, NJ (US); Corinne M. LE GOFF, Lawrenceville, NJ (US); Khursheed ANWER, Madison, AL (US)

(21) Appl. No.: 18/854,353

(22) PCT Filed: Apr. 6, 2023
(86) PCT No.: PCT/US2023/065481

§ 371 Date: Oct. 4, 2024

Related U.S. Application Data
(60) Provisional application No. 63/328,186, filed on Apr. 6, 2022.
(60) Provisional application No. 63/376,909, filed on Sep. 23, 2022.

(51) Int. Cl. A61K 39/215; A61K 39/00; A61K 39/02; A61P 37/04

(52) U.S. Cl. CPC A61K 39/215; A61K 39/02; A61P 37/04; A61K 2039/53; A61K 2039/55505; A61K 2039/55533; A61K 2039/55538; A61K 2039/55555; A61K 2039/6093

(57) ABSTRACT

Disclosed herein are immune stimulatory compositions, pharmaceutical compositions, and vaccines comprising a polynucleotide comprising at least one antigen nucleic acid which encodes at least one pathogen protein or an antigenic fragment thereof, wherein the antigen nucleic acid is operably linked to a first promoter; a delivery component selected from the group consisting of a cationic polymer, a poly-inosinic-polycytidylic acid, a poloxamer, or derivative thereof; and an adjuvant comprising an aluminum or aluminum-salt based adjuvant, a stimulator of interferon genes (STING) agonist, or a combination thereof. Methods of production and therapeutic use of the same are also disclosed herein.

### CROSS-REFERENCE TO RELATED APPLICATIONS

This application claims the benefit of U.S. Provisional Application No. 63/328,186, filed Apr. 6, 2022 and U.S. Provisional Application No. 63/376,909, filed Sep. 23, 2022, each of which are incorporated herein by reference in their entirety. REFERENCE TO SEQUENCE LISTING SUBMITTED ELECTRONICALLY

The content of the electronically submitted sequence listing in ASCII text file (Name: 2437_077PC02_Seglisting_ST26; Size: 263,436 bytes; and Date of Creation: Apr. 5, 2023, filed with the application is incorporated herein by reference in its entirety.

### FIELD

The present disclosure relates generally to immunology, vaccines, and gene therapy. In certain aspects, the disclosure relates to compositions and methods of generating an immune response to one or more viral antigens (e.g., SARS-CoV-2 antigens), bacterial antigens, or parasite antigens for treating, reducing the likelihood of, or preventing infection and disease in mammals.

### BACKGROUND OF THE DISCLOSURE

Vaccines including inactivated virus, antigen subunits, and nucleic acid (DNA and RNA) vaccines are being developed for a variety of infectious diseases across the globe. Most recently, cases of monkeypox virus infections have been increasing globably, prompting the need of new vaccines against the monkeypox virus. Also recently, severe acute respiratory syndrome coronavirus 2 (SARS-CoV-2) has sparked the development of RNA-based SARS-CoV-2 vaccines. In comparison to RNA vaccines, DNA vaccines can have the potential for better stability, durability, lower cost, and longer development history.

Further, DNA vaccines can have certain advantages over conventional inactivated or protein subunit vaccines due to their potential to generate humoral and cellular immunity and low risk of virulence and folding problems associated with inactivated viruses and subunit vaccines, respectively. Despite their attractiveness, issues such as suboptimal immunogenicity and effective delivery have been concerns with DNA vaccines.

Thus, a need remains for improved DNA vaccine compositions that are effective in prophylactic and therapeutic settings.

### BRIEF SUMMARY OF THE DISCLOSURE

Certain aspects of the disclosure are directed to an immune stimulatory composition comprising: (a) a polynucleotide (e.g., an expression vector) comprising an antigen nucleic acid which encodes a pathogen protein or an antigenic fragment thereof (e.g., a first pathogen protein or an antigenic fragment thereof), wherein the antigen nucleic acid is operably linked to a promoter (e.g., a first promoter); (b) a delivery component selected from the group consisting of a cationic polymer, a poly-inosinic-polycytidylic acid, a poloxamer, or derivative thereof; and (c) an adjuvant comprising an aluminum or aluminum-salt based adjuvant, a stimulator of interferon genes (STING) agonist, or a combination thereof.

In some aspects, the polynucleotide (e.g., an expression vector) comprises a single antigen nucleic acid which encodes a single pathogen protein or an antigenic fragment thereof. In some aspects, the polynucleotide (e.g., an expression vector) comprises two or more (e.g., two, three, four, five, six, seven or eight) antigen nucleic acids each encoding a different pathogen protein or antigenic fragment thereof.

In some aspects, the delivery component is crown poloxamer and the adjuvant is an aluminum or aluminum-salt based adjuvant. In some aspects, the delivery component is crown poloxamer and the adjuvant is a STING agonist. In some aspects, the delivery component is crown poloxamer and the adjuvant is an aluminum or aluminum-salt based adjuvant and a STING agonist.

In some aspects, the polynucleotide (e.g., an expression vector) comprises a single antigen nucleic acid encoding a single pathogen protein or antigenic fragment thereof and the delivery component is crown poloxamer. In some aspects, the polynucleotide (e.g., an expression vector) comprises a single antigen nucleic acid encoding a single pathogen proteins or antigenic fragments thereof; the delivery component is crown poloxamer; and the adjuvant is an aluminum or aluminum-salt based adjuvant and/or a STING agonist.

In some aspects, the polynucleotide (e.g., an expression vector) comprises at least two antigen nucleic acids encoding different pathogen proteins or antigenic fragments thereof and the delivery component is crown poloxamer. In some aspects, the polynucleotide (e.g., an expression vector) comprises at least two antigen nucleic acids encoding different pathogen proteins or antigenic fragments thereof; the delivery component is crown poloxamer; and the adjuvant is an aluminum or aluminum-salt based adjuvant and/or a STING agonist.

In some aspects, the aluminum or aluminum-salt based adjuvant is selected from the group consisting of an aluminum phosphate, an aluminum hydroxide, an aluminum oxyhydroxide, a potassium aluminum sulfate [KAl(SO4)2], an aluminum bicarbonate, an aluminum hydroxyphosphate, an aluminum hydroxyphosphate sulfate, an aluminum chloride, an aluminum silicate, and any combination thereof.

In some aspects, the aluminum or aluminum-salt based adjuvant comprises an aluminum phosphate, an aluminum hydroxide, a potassium aluminum sulfate [KAl(SO₄)₂], an aluminum oxyhydroxide, or any combination thereof.

In some aspects, the aluminum or aluminum-salt based adjuvant comprises an aluminum phosphate or an aluminum hydroxide.

In some aspects, aluminum salt-based adjuvant is a mixture of aluminum hydroxide and magnesium hydroxide, a mixture of aluminum sulfate and sodium hydroxide, a mixture of aluminum sulfate and potassium hydroxide, a mixture of aluminum phosphate and magnesium hydroxide, aluminum phosphate and sodium hydroxide, aluminum phosphate and potassium hydroxide or a mixture of aluminum phosphate and aluminum hydroxide.

In some aspects, the STING agonist is selected from the group consisting of a cyclic di-nucleotides, a non-cyclic di-nucleotide small molecule, an amidobenzimidazole (ABZI), a flavonoid, a nanovaccine, an antibody drug conjugate, a bacterial vector, and an ENPP1 inhibitor.

In some aspects, the STING agonist is cyclic guanosine monophosphate (cGMP), cyclic adenosine monophosphate (cAMP), or cyclic guanosine monophosphate-adenosine monophosphate (cGAMP). In some aspects, the STING agonist is cGMP.

In some aspects, the composition comprises an adjuvant selected from an unmethylated cytosine-guanine dinucleotide-containing oligonucleotide (CpG), a M59 (oil-in-water emulsion of squalene oil), ASO3 (α-tocopherol, squalene, and polysorbate 80 in an oil-in-water emulsion), or any combination thereof. In some aspects, the adjuvant comprises one or more CpG-containing oligonucleotides. In some aspects, the adjuvant comprises M59 (oil-in-water emulsion of squalene oil). In some aspects, the adjuvant comprises ASO3 (α-tocopherol, squalene, and polysorbate 80 in an oil-in-water emulsion).

In some aspects, the adjuvant comprises a STING agonist and one or more CpG-containing oligonucleotides. In some aspects, the adjuvant comprises cGMP and one or more CpG-containing oligonucleotides.

In some aspects, the adjuvant comprises a STING agonist and M59.

In some aspects, the adjuvant comprises a STING agonist and ASO3.

In some aspects, the adjuvant comprises aluminum salt-based adjuvant and one or more CpG-containing oligonucleotides. In some aspects, the adjuvant comprises aluminum salt-based adjuvant and M59. In some aspects, the adjuvant comprises aluminum salt-based adjuvant and ASO3.

In some aspects, the antigen nucleic acid (e.g a first antigen nucleic acid) of the polynucleotide encodes the at least one pathogen protein (e.g., one or more pathogen antigens) or an antigenic fragment thereof (e.g., a first pathogen protein or an antigenic fragment thereof) which is selected from the group consisting of a viral protein, a bacterial protein, a parasite protein, and any antigenic fragments thereof.

In some aspects, the polynucleotide further comprises at least one additional antigen nucleic acid (e.g., a second antigen nucleic acid), which encodes at least one additional pathogen protein (e.g., one or more pathogen antigens) or an antigenic fragment thereof (e.g., a second pathogen protein or an antigenic fragment thereof). In some aspects, the polynucleotide further comprises a further additional antigen nucleic acid (e.g., a third antigen nucleic acid), which encodes a further additional pathogen protein (e.g., a third pathogen antigen) or an antigenic fragment thereof (e.g., a third pathogen protein or an antigenic fragment thereof).

In some aspects, the at least one additional pathogen protein or an antigenic fragment thereof (e.g., a second pathogen protein) is selected from the group consisting of a viral protein, a bacterial protein, a parasite protein, and any antigenic fragments thereof.

In some aspects, the at least one pathogen protein or antigenic fragment thereof (e.g., first pathogen protein and/or the second pathogen protein) is/are selected from the group consisting of a Yersinia pestis antigen, a Mycobacterium tuberculosis antigen, a Meningococcus antigen, an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a Rabies virus antigen, a Cytomegalovirus antigen, a Yellow fever virus antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a orthopoxvirus antigen, a monkeypox antigen, a vaccinia antigen, a smallpox antigen, a Epstein bar virus antigen, a nipha virus antigen, a varicella-zoster virus antigen, a Clostridioides difficile antigen, a Streptococcus pneumonia antigen, a Neisseria meningitides antigen, a Toxoplasma gondii antigen, a Plasmodium falciparum antigen, antigenic fragments thereof, and any combinations thereof.

In some aspects, at least one pathogen protein or antigenic fragment thereof (e.g., first pathogen protein and/or the second pathogen protein) is/are selected from the group consisting of: a Yersinia pestis F1-Ag, a Yersinia pestis V-Ag, a Mycobacterium tuberculosis Apa antigen, a Mycobacterium tuberculosis HP65 antigen, a Mycobacterium tuberculosis rAg85A antigen, an E71 VP1 antigen, a GST-tagged E71-VP1 antigen, a Cox protein antigen, a GST-tagged Cox protein antigen, an HSV-1 envelope antigen, an HSV-2 envelope antigen, an HSV-2 gB2 antigen, an HSV-2 gC2 antigen, an HSV-2 gD2 antigen, an HSV-2 gE2 antigen, an HIV Env antigen, an HIV Gag antigen, an HIV Nef antigen, an HIV Pol antigen, an HPV minor capsid protein L2 antigen, a human papillomavirus type 16 Regulatory protein E2 antigen, a human papillomavirus type 16 Protein E6 antigen, a human papillomavirus type 16 Protein E7 antigen, a human papillomavirus type 18 Regulatory protein E2 antigen, a human papillomavirus type 18 Protein E6 antigen, a human papillomavirus type 18 Protein E7 antigen, a human papillomavirus type 6a Regulatory protein E2 antigen, a human papillomavirus type 6a Protein E6 antigen, a human papillomavirus type 6a Protein E7 antigen, a human papillomavirus 11 Regulatory protein E2 antigen, a human papillomavirus 11 Protein E6 antigen, a human papillomavirus 11 Protein E7 antigen, an HCV NS3 antigen, a hepatitis C virus genotype 1a Genome polyprotein antigen, a hepatitis C virus genotype 1b Genome polyprotein antigen, a hepatitis C virus genotype 2a Genome polyprotein antigen, a hepatitis C virus genotype 3a Genome polyprotein antigen, a RSV F antigen, a RSV G antigen, a Dengue virus E protein antigen, a Dengue virus EDIII antigen, a Dengue virus NS1 antigen, a Dengue virus DEN-80E antigen, an Ebola virus GB antigen, an Ebola virus VP24 antigen, an Ebola virus VP40 antigen, an Ebola virus NP antigen, an Ebola virus VP30 antigen, an Ebola virus VP35 antigen, a Zika virus envelope domain III antigen, a Zika virus CKD antigen, a Chikungunya virus E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), a measles virus hemagglutinin protein MV-H antigen, a measles virus fusion protein MV-F antigen, a MERS-CoV S protein antigen, an antigen from the receptor-binding domain of the MERS-CoV S protein, an antigen from the membrane fusion domain of the MERS-CoV S protein, a SARS-CoV S protein antigen, an antigen from the receptor binding domain of the SARS-CoV S protein, an antigen from the membrane fusion domain of the SARS-CoV S protein, a SARS-CoV E protein antigen, a SARS-CoV M protein antigen, a Delta variant SARS CoV-2 spike (S) protein, an Omicron variant SARS CoV-2 spike (S) protein, a Delta variant SARS-CoV-2 membrane (M) protein, an Omicron variant SARS-CoV-2 membrane (M) protein, a Delta variant SARS-CoV-2 envelope (E) protein, an Omicron variant SARS-CoV-2 envelope (E) protein, a Delta variant SARS-CoV-2 nucleocapsid (N) protein, an Omicron variant SARS-CoV-2 nucleocapsid (N) protein, a orthopoxvirus antigen, a monkeypox A35R protein antigen, a monkeypox H3L protein antigen, a monkeypox L1R protein antigen, a Clostridioides difficile 630 spore coat protein: peroxiredoxin/chitinase antigen, a Clostridioides difficile 630 flagellin C antigen, a Clostridioides difficile Surface layer protein A (Fragment) antigen, an Epstein-Barr virus (strain B95-8) nuclear antigen 1 antigen, an Epstein-Barr virus (strain B95-8) Envelope glycoprotein B antigen, an Epstein-Barr virus (strain B95-8) Envelope glycoprotein H antigen, an Epstein-Barr virus (strain B95-8) Envelope glycoprotein GP350 antigen, an Epstein-Barr virus (strain B95-8) Latent membrane protein 1 antigen, an Epstein-Barr virus (strain B95-8) Latent membrane protein 2 antigen, a Neisseria meningitides Factor H-binding protein antigen, a Neisseria meningitidis serogroup B Neisseria adhesin A antigen, a Neisseria meningitidis Neisserial heparin binding antigen antigen, a Vaccinia virus (strain Western Reserve) Protein A27 antigen, a Vaccinia virus (strain Western Reserve)EEV membrane phosphoglycoprotein antigen, a Vaccinia virus B5R (Fragment) antigen, a Vaccinia virus Envelope protein H3 antigen, a Vaccinia virus (strain Western Reserve) IMV membrane protein antigen, a Nipah virus Fusion glycoprotein FO antigen, a Nipah virus Glycoprotein G antigen, a Varicella-zoster virus (strain Dumas) Envelope glycoprotein E antigen, a Toxoplasma gondii MIC8 antigen, a Plasmodium falciparum SERA5 polypeptide antigen, a Plasmodium falciparum circumsporozite protein antigen, antigenic fragments thereof, and any combination thereof.

In some aspects, the at least one pathogen protein or antigenic fragment thereof (e.g., first pathogen protein and/or the second pathogen protein) is a SARS-CoV-2 antigen or an antigenic fragment thereof. In some aspects, the at least one pathogen protein or antigenic fragment thereof (e.g., first pathogen protein) is a SARS-CoV-2 protein or an antigenic fragment thereof selected from the group consisting of: a SARS CoV-2 spike (S) protein, a SARS-CoV-2 membrane (M) protein, a SARS-CoV-2 envelope (E) protein, a SARS-CoV-2 nucleocapsid (N) protein, or an antigenic fragment thereof, and wherein the at least one additional pathogen protein or antigenic fragment thereof (e.g., the second pathogen protein) is a SARS-CoV-2 protein or an antigenic fragment thereof selected from the group consisting of: a SARS CoV-2 spike (S) protein, a SARS-CoV-2 membrane (M) protein, a SARS-CoV-2 envelope (E) protein, a SARS-CoV-2 nucleocapsid (N) protein, or an antigenic fragment thereof. In some aspects, the at least one pathogen protein or antigenic fragment thereof (e.g., first pathogen protein and/or the second pathogen protein) is a monkeypox antigen or an antigenic fragment thereof. In some aspects, the at least one pathogen protein or antigenic fragment thereof (e.g., first pathogen protein) is a monkeypox protein or an antigenic fragment thereof selected from the group consisting of: a monkeypox A35R protein, a monkeypox H3L protein, a monkeypox L1R protein, or an antigenic fragment thereof, and wherein the at least one additional pathogen protein or antigenic fragment thereof (e.g., the second pathogen protein) is a SARS-CoV-2 protein or an antigenic fragment thereof selected from the group consisting of: is a monkeypox protein or an antigenic fragment thereof selected from the group consisting of: a monkeypox A35R protein, a monkeypox H3L protein, a monkeypox L1R protein, or an antigenic fragment thereof.

In some aspects, the at least one pathogen protein or antigenic fragment thereof (e.g., first pathogen protein and/or the second pathogen protein) is selected from an Influenza A type antigen, an Influenza B type antigen, an Influenza C type antigen, and an Influenza D type antigen. In some aspects, the at least one pathogen protein or antigenic fragment thereof (e.g., first pathogen protein and/or the second pathogen protein) is selected from the group consisting of an influenza virus hemagglutinin (HA) antigen, an influenza virus neuraminidase (NA) antigen, an influenza virus matrix-1 (M1) protein antigen, an influenza virus matrix-2 (M2) protein antigen, an influenza RNA polymerase subunit PB1 antigen, an influenza RNA polymerase subunit PB2 antigen, an influenza RNA polymerase subunit PA antigen, an influenza non-structural protein 1 (NS1) antigen, an influenza non-structural protein 2 (NS2) protein antigen, antigenic fragments thereof, and any combination thereof.

In some aspects, the antigen nucleic acid of the polynucleotide encodes a SARS CoV-2 S protein or an antigenic fragment thereof. In some aspects, the antigen nucleic acid of the polynucleotide encodes a pathogen protein or antigenic fragment thereof that is selected from the group consisting of: a SARS-CoV-2 M protein or an antigenic fragment thereof, a SARS-CoV-2 E protein or an antigenic fragment thereof, a SARS-CoV-2 N protein or an antigenic fragment thereof, and any combination thereof. In some aspects, the antigen nucleic acid of the polynucleotide encodes a monkeypox A35R protein or an antigenic fragment thereof. In some aspects, the antigen nucleic acid of the polynucleotide encodes a monkeypox H3L protein or an antigenic fragment thereof. In some aspects, the antigen nucleic acid of the polynucleotide encodes a monkeypox L1R protein or an antigenic fragment thereof.

In some aspects, the at least one pathogen protein or antigenic fragment thereof (e.g., first pathogen protein) is selected from a SARS-CoV-2 protein or an antigenic fragment thereof selected from the group consisting of: a SARS CoV-2 spike (S) protein, a SARS-CoV-2 membrane (M) protein, a SARS-CoV-2 envelope (E) protein, a SARS-CoV-2 nucleocapsid (N) protein, or an antigenic fragment thereof, and wherein the at least one additional pathogen protein or antigenic fragment thereof (e.g., second pathogen protein) is selected from a SARS-CoV-2 protein or an antigenic fragment thereof selected from the group consisting of: a SARS CoV-2 spike (S) protein, a SARS-CoV-2 membrane (M) protein, a SARS-CoV-2 envelope (E) protein, a SARS-CoV-2 nucleocapsid (N) protein, or an antigenic fragment thereof. In some aspects, the at least one pathogen protein or antigenic fragment thereof (e.g., first pathogen protein) is selected from a monkeypox protein or an antigenic fragment thereof selected from the group consisting of: a monkeypox A35R protein, a monkeypox H3L protein, a monkeypox L1R protein, or an antigenic fragment thereof, and wherein the at least one additional pathogen protein or antigenic fragment thereof (e.g., second pathogen protein) is selected from a monkeypox protein or an antigenic fragment thereof selected from the group consisting of: a monkeypox A35R protein, a monkeypox H3L protein, a monkeypox LIR protein, or an antigenic fragment thereof.

In some aspects, the at least one pathogen protein or antigenic fragment thereof (e.g., first pathogen protein) is a SARS-CoV-2 S protein or an antigenic fragment thereof, and wherein the at least one additional pathogen protein or antigenic fragment thereof (e.g., second pathogen protein) is a SARS-CoV-2 S protein or an antigenic fragment thereof, and wherein the at least one pathogen protein or antigenic fragment thereof and the at least one additional pathogen protein or antigenic fragment thereof (e.g., first and second pathogen proteins) are derived from different strains of SARS-CoV-2.

In some aspects, the at least one pathogen protein or antigenic fragment thereof (e.g., first pathogen protein) is a monkeypox protein (e.g., A35R, H3L, or L1R protein) or an antigenic fragment thereof, and wherein the at least one additional pathogen protein or antigenic fragment thereof (e.g., second pathogen protein) is a second monkeypox protein (e.g., A35R, H3L, or L1R protein) or an antigenic fragment thereof, and wherein the at least one pathogen protein or antigenic fragment thereof and the at least one additional pathogen protein or antigenic fragment thereof (e.g., first and second pathogen proteins) are derived from different strains of monkeypox.

In some aspects, the at least one additional antigen nucleic acid (e.g., second antigen nucleic acid) of the polynucleotide is operably linked to the first promoter through an internal ribosome entry site (IRES) sequence.

In some aspects, the at least one additional antigen nucleic acid (e.g., second antigen nucleic acid) of the polynucleotide is operably linked to one or more additional promoters (e.g., a second promoter). In some aspects, the second additional antigen nucleic acid (e.g., third antigen nucleic acid) of the polynucleotide is operably linked to one or more additional promoters (e.g., a third promoter).

In some aspects, the promoter (e.g., first promoter) or the one or more additional promoters (e.g., second promoter and/or third promoter) is/are selected from the group consisting of: a cytomegalovirus (CMV) promoter, a Rouse sarcoma virus (RSV) promoter, a Moloney murine leukemia virus (Mo-MuLV) long terminal repeat (LTR) promoter, a human ubiquitin C promoter, a mammalian elongation factor 1 (EF1) promoter, a human elongation factor 1a/Human T cell Leukemia Virus Type 1 Long Terminal Repeat (hEF1/HTLV) promoter, a cytokeratin 18 (CK18) promoter, a cytokeratin 19 (CK19) promoter, a simian virus 40 (SV40) promoter, a murine U6 promoter, a skeletal α-actin promoter, a β-actin promoter, a murine phosphoglycerate kinase 1 (PGK1) promoter, a human PGK1 promoter, a CBA promoter, a CAG promoter, and any combination thereof.

In some aspects, the antigen nucleic acid (e.g., first, second, or third antigen nucleic acid) of the polynucleotide encodes a polypeptide comprising the amino acid sequence of SEQ ID NO: 2, SEQ ID NO: 4, SEQ ID NO: 115, SEQ ID NO: 117, SEQ ID NO: 119, SEQ ID NO: 121, SEQ ID NO: 125, or SEQ ID NO: 127.

In some aspects, the antigen nucleic acid (e.g., first, second, or third antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 1, SEQ ID NO: 3, SEQ ID NO: 114, SEQ ID NO: 116, SEQ ID NO: 118, SEQ ID NO: 120, SEQ ID NO: 124, or SEQ ID NO: 126.

In some aspects, the antigen nucleic acid (e.g., first, second, or third antigen nucleic acid) of the polynucleotide encodes the receptor binding domain (RBD) of the SARS-Cov-2 S protein or an antigenic fragment thereof.

In some aspects, the antigen nucleic acid (e.g., first, second, or third antigen nucleic acid) of the polynucleotide encodes a polypeptide comprising the amino acid sequence of SEQ ID NO: 6.

In some aspects, the antigen nucleic acid (e.g., first, second, or third antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 5.

In some aspects, the antigen nucleic acid (e.g., first, second, or third antigen nucleic acid) of the polynucleotide encodes the S1 subunit of the SARS-Cov-2 S protein or an antigenic fragment thereof.

In some aspects, the antigen nucleic acid (e.g., first, second, or third antigen nucleic acid) of the polynucleotide encodes a polypeptide comprising the amino acid sequence of SEQ ID NO: 40.

In some aspects, the antigen nucleic acid (e.g., first, second, or third antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 39.

In some aspects, the at least one additional antigen nucleic acid (e.g., second and/or third antigen nucleic acid) of the polynucleotide encodes a polypeptide comprising the amino acid sequence of SEQ ID NO: 8, SEQ ID NO: 10 SEQ ID NO: 12, SEQ ID NO: 14, SEQ ID NO: 16, SEQ ID NO: 18, or SEQ ID NO: 20.

In some aspects, the at least one additional antigen nucleic acid (e.g., second and/or third antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 7, SEQ ID NO: 9, SEQ ID NO: 11, SEQ ID NO: 13, SEQ ID NO: 15, SEQ ID NO: 17, SEQ ID NO: 19, or SEQ ID NO: 131.

In some aspects, the at least one additional antigen nucleic acid (e.g., second and/or third antigen nucleic acid) of the polynucleotide encodes a polypeptide comprising the amino acid sequence of SEQ ID NO: 22, SEQ ID NO: 24, or SEQ ID NO: 26.

In some aspects, the at least one additional antigen nucleic acid (e.g., second and/or third antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 21, SEQ ID NO: 23, or SEQ ID NO: 25.

In some aspects, the at least one additional antigen nucleic acid (e.g., second and/or third antigen nucleic acid) of the polynucleotide encodes a polypeptide comprising the amino acid sequence of SEQ ID NO: 28 or SEQ ID NO: 123.

In some aspects, the at least one additional antigen nucleic acid (e.g., second and/or third antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 27 or SEQ ID NO: 122.

In some aspects, the at least one additional antigen nucleic acid (e.g., second and/or third antigen nucleic acid) of the polynucleotide encodes a polypeptide comprising the amino acid sequence of SEQ ID NO: 67, SEQ ID NO: 69, SEQ ID NO: 71, SEQ ID NO: 72, SEQ ID NO: 73, or SEQ ID NO: 74.

In some aspects, the at least one additional antigen nucleic acid (e.g., second and/or third antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 66, SEQ ID NO: 68, or SEQ ID NO: 70.

In some aspects, the polynucleotide further comprises a nucleic acid sequence encoding one or more immune modifier proteins. In some aspects, the immune modifier protein is a cytokine or a chemokine. In some aspects, the immune modifier protein comprises: IL-2, IL-12 p35, IL-12 p40, IL-12 p70, IL-15, IL-18, TNFα, GM-CSF, IFN-α, IFN-β, MHC I, MHC II, HLA-DR, CD80, CD86, or any combination thereof. In some aspects, the nucleic acid sequence encoding the one or more immune modifier proteins is operably linked to a promoter.

In some aspects, the polynucleotide further comprises one or more post-transcriptional regulatory elements. In some aspects, the post-transcriptional regulatory element is a wood chuck hepatitis virus post-transcriptional regulatory element (WPRE).

In some aspects, the polynucleotide further comprises at least one 3′ UTR poly(a) tail sequence operably linked to the first antigen nucleic acid, the second antigen nucleic acid, or any combination thereof.

In some aspects, the 3′ UTR poly(a) tail sequence is a 3′ UTR SV40 poly(a) tail sequence, a 3′ UTR bovine growth hormone (bGH) poly(A) sequence, a 3′ UTR actin poly(A) tail sequence, a 3′ UTR hemoglobin poly(A) sequence, or combinations thereof.

In some aspects, the polynucleotide further comprises an enhancer sequence. In some aspects, the enhancer sequence comprises a human actin enhancer sequence, a human myosin enhancer sequence, a human hemoglobin enhancer sequence, a human muscle creatine enhancer sequence, a viral enhancer sequence, a polynucleotide function enhancer sequence, or any combination thereof.

In some aspects, the enhancer sequence comprises a CMV intronic sequence, a (3-actin intronic sequence, or the combination thereof. In some aspects, the enhancer sequence is a CMV intronic sequence. In some aspects, the enhancer sequence is a CMV intronic sequence, a SV40 enhancer sequence, a β-actin intronic sequence, or combinations thereof.

In some aspects, the polynucleotide is within a vector, wherein the vector is a DNA plasmid, a viral vector, a bacterial vector, a cosmid, or an artificial chromosome. In some aspects, the vector is a DNA plasmid. In some aspects, the DNA plasmid vector is selected from the group consisting of: pVac 1, pVac 2, pVac 3, pVac 4, pVac 5, pVac 6, pVac 7, pVac 8 pVac 9, pVac 10 pVac 11, pVac 12, pVac 13, pVac 14, pVac 15 pVac 16, pVac 17, pVac 18, pVac 19, pVac 20, pVac 21, pVac 22, pVac 23, pVac 24, pVac 25, pVac 26, pVac 27, pVac 28, pVac 29, pVac 30, pVac 31, pVac 32, pVac 33, pVac 34, pVac 35, pVac 36, pVac 37, pVac 38, and pH1N1 Brisbane (FIG. 14B to 14 AO).

In some aspects, the composition is a pharmaceutical composition comprising a pharmaceutically acceptable carrier. In some aspects, the composition or pharmaceutical composition is a vaccine.

In some aspects, the cationic polymer comprises a synthetic functionalized polymer, a 3-amino ester, a lipid, a lipopolymer, or a chemical derivative thereof.

In some aspects, the synthetic functionalized polymer is a biodegradable cross-linked cationic multi-block copolymer.

In some aspects, the biodegradable cross-linked cationic multi-block copolymer is represented by the formula: (CP)xLyYz, wherein: (a) CP represents a cationic polymer containing at least one secondary amine group, wherein the cationic polymer has a number averaged molecular weight within the range of 1,000 to 25,000 Dalton, (b) Y represents a bifunctional biodegradable linker containing ester, amide, disulfide, or phosphate linages, (c) L represents a ligand, (d) x is an integer in the range from 1 to 20, (e) y is an integer in the range from 0 to 100, and (f) z is an integer in the range from 0 to 40.

In some aspects, the cationic polymer comprises biodegradable cross-linked linear polyethyleneimine (LPEI).

In some aspects, the bifunctional biodegradable linker is hydrophilic and comprises a biodegradable linkage comprising a disulfide bond.

In some aspects, the bifunctional biodegradable linker is a dithiodipropionyl linker.

In some aspects, the biodegradable cross-linked cationic multi-block copolymer comprises LPEI and a dithiodipropionyl linker for cross-linking the multi-block copolymer, wherein the LPEI has an average molecular weight of 1,000 to 25,000 Dalton.

In some aspects, the biodegradable cross-linked cationic multi-block copolymer is covalently linked to at least one ligand.

In some aspects, the ligand is a targeting ligand selected from the group consisting of: a sugar moiety, a polypeptide, folate, and an antigen.

In some aspects, the sugar moiety is a monosaccharide or an oligosaccharide. In some aspects, the monosaccharide is galactose.

In some aspects, the polypeptide is a glycoprotein, an antibody, an antibody fragment, a cell receptor, a cytokine receptor, or a growth factor receptor. In some aspects, the growth factor receptor is an epidermal growth factor receptor. In some aspects, the glycoprotein is transferrin or asialoorosomucoid (ASOR).

In some aspects, the antigen is a viral antigen, a bacterial antigen, or a parasite antigen.

In some aspects, the biodegradable cross-linked cationic multi-block copolymer is covalently linked to polyethylene glycol (PEG) of molecular weight ranging from 500 to 20,000 Dalton.

In some aspects, the biodegradable cross-linked cationic multi-block copolymer is covalently linked to a fatty acyl chain selected from the group consisting of: oleic acid, palmitic acid, and stearic acid.

In some aspects, the biodegradable cross-linked cationic multi-block copolymer comprises at least one amine group that is electrostatically attracted to a polyanionic compound.

In some aspects, the polyanionic compound is a nucleic acid, wherein the biodegradable cross-linked cationic multi-block copolymer condenses the nucleic acid to form a compact structure.

In some aspects, the lipopolymer is a cationic lipopolymer comprising a PEI backbone covalently linked to a lipid or a PEG. In some aspects, the PEI backbone is covalently linked to a lipid and a PEG. In some aspects, the lipid and the PEG are directly attached to the PEI backbone by covalent bonds. In some aspects, the lipid is attached to the PEI backbone through a PEG spacer. In some aspects, the PEG has a molecular weight of between 50 to 20,000 Dalton. In some aspects, the molar ratio of PEG to PEI is within a range of 0.1:1 to 500:1. In some aspects, the molar ratio of the lipid to the PEI is within a range of 0.1:1 to 500:1.

In some aspects, the lipid is a cholesterol, a cholesterol derivative, a C12 to C18 fatty acid, or a fatty acid derivative. In some aspects, the PEI is covalently linked to cholesterol and PEG, and wherein the average PEG:PEI:cholesterol molar ratio in the cationic lipopolymer is within the range of 1-5 PEG:1 PEI:0.4-1.5 cholesterol. In some aspects, the PEI has a linear or branch configuration with a molecular weight of 100 to 500,000 Dalton.

In some aspects, the cationic lipopolymer further comprises a pendant functional moiety selected from the group consisting of: a receptor ligand, a membrane permeating agent, an endosomolytic agent, a nuclear localization sequence, and a pH sensitive endosomolytic peptide.

In some aspects, the cationic lipopolymer further comprises a targeting ligand, wherein the targeting ligand is directly attached to the PEI backbone or is attached through a PEG linker. In some aspects, the targeting ligand is selected from the group consisting of: a sugar moiety, a polypeptide, folate, and an antigen. In some aspects, the sugar moiety is a monosaccharide or an oligosaccharide. In some aspects, the monosaccharide is galactose. In some aspects, the polypeptide is a glycoprotein, an antibody, an antibody fragment, a cell receptor, a cytokine receptor, or a growth factor receptor.

In some aspects, the cationic polymer is present in an amount sufficient to produce a ratio of amine nitrogen in the cationic polymer to phosphate in the DNA plasmid vector from about 0.01:1 to about 50:1. In some aspects, the ratio of amine nitrogen in the cationic polymer to phosphate in the DNA plasmid vector from about 1:10 to about 10:1.

In some aspects, composition, pharmaceutical composition, or vaccine comprises about 0.1 mg/ml to about 10.0 mg/ml nucleic acid complexed with the cationic polymer.

In some aspects, the delivery component comprises a lipopolyamine with the following formula:

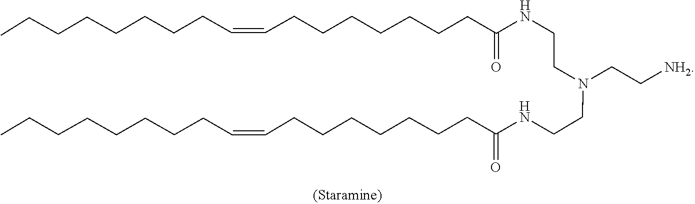 [SMILES: CCCCCCCC/C=C\CCCCCCCC(=O)NCCN(CCN)CCNC(=O)CCCCCCC/C=C\CCCCCCCC]

In some aspects, the delivery component comprises a mixture of the lipopolyamine and an alkylated derivative of the lipopolyamine.

In some aspects, the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride.

In some aspects, the alkylated derivative of the lipopolyamine has the following formula:

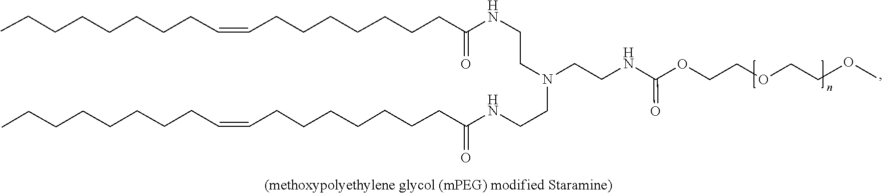

wherein n represents an integer from 10 to 100 repeating units containing of 2-5 carbon atoms each.

In some aspects, the ratio of the lipopolyamine to the alkylated derivative of the lipopolyamine in the mixture is 1:1 to 10:1.

In some aspects, the lipopolyamine is present in an amount sufficient to produce a ratio of amine nitrogen in the lipopolyamine to phosphate in the DNA plasmid vector from about 0.01:1 to about 50:1.

In some aspects, the lipopolyamine is present in an amount sufficient to produce a ratio of amine nitrogen in the lipopolyamine to phosphate in the DNA plasmid vector from about 1:10 to about 10:1.

In some aspects, the delivery component comprises a lipopolyamine with the following formula:

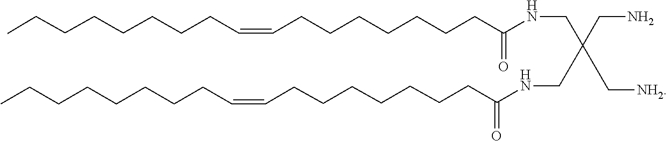 [SMILES: CCCCCCCC/C=C\CCCCCCCC(=O)NCC(CN)(CN)CNC(=O)CCCCCCC/C=C\CCCCCCCC]

In some aspects, the delivery component comprises a mixture of the lipopolyamine and an alkylated derivative of the lipopolyamine.

In some aspects, the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride.

In some aspects, the ratio of the lipopolyamine to the alkylated derivative of the lipopolyamine in the mixture is 1:1 to 10:1.

In some aspects, the lipolyamine is present in an amount sufficient to produce a ratio of amine nitrogen in the lipopolyamine to phosphate in the DNA plasmid vector from about 0.01:1 to about 50:1.

In some aspects, the lipolyamine is present in an amount sufficient to produce a ratio of amine nitrogen in the lipopolyamine to phosphate in the DNA plasmid vector from about 1:10 to about 10:1.

In some aspects, the delivery component comprises a poloxamer with the following formula:

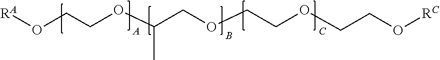

or a pharmaceutically acceptable salt thereof, wherein:
A represents an integer from 2 to 141;
B represents an integer from 16 to 67;
C represents an integer from 2 to 141;
RA and RC are the same or different, and are R′-L- or H, wherein at least one of RA and RC is R′-L-;
L is a bond, —CO—, —CH2-O—, or —O—CO—; and
R′ is a metal chelator.

In some aspects, the delivery component comprises a poloxamer with the following formula:

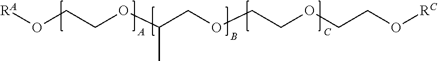

or a pharmaceutically acceptable salt thereof, wherein:
A represents an integer from 2 to 141;
B represents an integer from 16 to 67;
C represents an integer from 2 to 141;
RA and RC are the same or different, and are R′-L- or H;
L is a bond, —CO—, —CH2-O, or —O—CO—; and
R′ is a metal chelator.

In some aspects, at least one of RA and RC is R′-L-.

In some aspects, the R′ is covalently bound to the poloxamer (e.g., where a metal chelator (e.g., a crown) is covalently attached at different density to the poloxamer).

In some aspects, one metal chelator or two or more metal chelators is/are bound to the poloxamer.

In some aspects, 2-100 metal chelators are bound to the poloxamer.

In some aspects, the metal chelator is RNNH—, RN2N—, or (R″—(N(R″)—CH2CH2)x)2-N—CH2CO—, wherein each x is independently 0-2, and wherein R″ is HO2-C—CH2-.

In some aspects, the metal chelator is a crown ether, a substituted-crown ether, a cryptand, or a substituted-cryptand.

In some aspects, the delivery component further comprises a PEG-PEI-cholesterol (PPC) lipopolymer, benzalkonium chloride (BAK), Omnifect, or a linear polyethyleneimine (LPEI). In some aspects, the LPEI is BD15-12.

In some aspects, the poloxamer (e.g, crown poloxamer) is present in a solution with the polynucleotide or DNA plasmid vector from about 0.01%-about 5%.

In some aspects, the solution is co-formulated with a metal chelator (e.g., where the co-formulated metal chelator is a free metal chelator, which is formulated with the poloxamer (e.g., a non-crown poloxamer)).

In some aspects, the co-formulated metal chelator is present in the solution at a concentration of about 0.1 mg/mL to about 20 mg/mL.

In some aspects, the co-formulated metal chelator is crown ether, a substituted-crown ether, a cryptand, or a substituted-cryptand.

In some aspects, the metal chelator or co-formulated metal chelator is crown ether (Aza-18-crown-6).

In some aspects, the delivery component comprises BD15-12. In some aspects, the nucleotide to polymer (N:P) ratio is 0.1:1 to 5:1.

In some aspects, the delivery component is PEG-PEI-cholesterol (PPC) lipopolymer. In some aspects, the nucleotide to polymer (N:P) ratio is 0.1:1 to 5:1.

In some aspects, the delivery component comprises Omnifect. In some aspects, the nucleotide to polymer (N:P) ratio is 0.1:1 to 5:1.

In some aspects, the delivery component comprises crown poloxamer connected by covalent bond directly or through a linker to an aluminum or aluminum-salt based adjuvant.

In some aspects, the delivery component comprises Staramine and mPEG modified Staramine. In some aspects, the mPEG modified Staramine is Staramine-mPEG515 or Staramine-mPEG11. In some aspects, the delivery component comprises Crossamine (N,N′-dioleoyl tetrakis(aminomethyl)methane).

In some aspects, the delivery component is Starmine. In some aspects, the nucleotide to polymer (N:P) ratio is 0.1:1 to 5:1.

In some aspects, the delivery component is Crossamine. In some aspects, the nucleotide to polymer (N:P) ratio is 0.1:1 to 5:1.

In some aspects, the ratio of Staramine to mPEG modified Staramine is 10:1. In some aspects, the nucleotide to polymer (N:P) ratio is 0.1:1 to 5:1.

In some aspects, the delivery component further comprises crown poloxamer.

In some aspects, the composition, pharmaceutical composition, or vaccine is stable at 0° C. to 5° C. for at least about 1 month, about 2 months, about 3 months, about 4 months, about 5 months, about 6 months, about 7 months, about 8 months, about 9 months, about 10 months, about 11 months, about 12 months, about 24 months or about 36 months. In some aspects, the composition, pharmaceutical composition, or vaccine is stable at 25° C. for at least about 7 days, about 10 day, or about 14 days, or about 60 days.

In some aspects, the composition, pharmaceutical composition, or vaccine is stable at −20° C. for at least about 1 month, about 2 months, about 3 months, about 4 months, about 5 months, about 6 months, about 7 months, about 8 months, about 9 months, about 10 months, about 11 months, about 12 months, about 24 months or about 36 months. 60

In some aspects, the composition is lyophilized and is substantially free of aqueous components. In some aspects, the composition is reconstituted with a diluent. In some aspects, the diluent is water.

Certain aspects of the disclosure are directed to a kit comprising the composition, pharmaceutical composition, or vaccine disclosed herein. In some aspects, the kit further comprises a glass vial. In some aspects, the kit further comprises instructions for using the composition or lyophilized composition in a method for inducing an immune response in a subject. In some aspects, the kit further comprises instructions for using the composition or lyophilized composition in a method for preventing, reducing the incidence of, attenuating or treating an infection in a subject.

In some aspects, the infection is a viral infection, a bacterial infection, or a parasite infection. In some aspects, the infection is a SARS-CoV-2 infection. In some aspects, the infection is an orthopox virus infection. In some aspects, the infection is a monkeypox virus infection. In some aspects, the infection is a vaccina virus infection. In some aspects, the infection is a smallpox virus infection. In some aspects, the infection is an Epstein bar virus infection. In some aspects, the infection is a nipha virus infection. In some aspects, the infection is a varicella-zoster virus infection. In some aspects, the infection is a Clostridioides difficile infection. In some aspects, the infection is a Streptococcus pneumoniae infection. In some aspects, the infection is a Neisseria meningitidis infection. In some aspects, the infection is influenza infection. In some aspects, the infection is a Yersinia pestis infection, a Mycobacterium tuberculosis infection, a Meningococcus infection, an enterovirus infection, a herpes simplex virus (HSV) infection, a human immunodeficiency virus (HIV) infection, a human papillomavirus (HPV) infection, a hepatitis C virus (HCV) infection, a respiratory syncytial virus (RSV) infection, a Rabies virus infection, a Cytomegalovirus infection, a Yellow fever virus infection, a dengue virus infection, an Ebola virus infection, a Zika virus infection, a chikungunya virus infection, a measles virus infection, a Middle East Respiratory Syndrome Coronavirus (MERS-CoV) infection, a Toxoplasma gondii infection, or a Plasmodium falciparum infection.

Certain aspects of the disclosure are directed to methods of inducing an immune response in a subject, the method comprising administering an effective amount of the composition, pharmaceutical composition, or vaccine disclosed herein. In some aspects, the immune response is to one or more SARS-CoV-2 antigens. In some aspects, the immune response is to one or more monkeypox antigens. In some aspects, the immune response is a protective immune response.

Certain aspects of the disclosure are directed to methods of preventing, reducing the incidence of, attenuating or treating an infection in a subject, the method comprising administering an effective amount of the composition, pharmaceutical composition, or vaccine disclosed herein.

In some aspects, the composition, pharmaceutical composition, or vaccine is administered to the subject by an intramuscular, transdermal, subcutaneous, intralymphatic, intranasal, or intraperitoneal route of administration. In some aspects, the composition, pharmaceutical composition, or vaccine is administered once, twice, three times, four times, five times, six times, or between seven and twenty times. In some aspects, the composition, pharmaceutical composition, or vaccine is administered more than once in an interval of from 1 day to about 21 days; about 2 days to about 18 days; about 3 days to about 14 days; or about 7 days; about 10 days; about 14 days; about 21 days; about 28 days; about 35 days; about 42 days; about 49 days or about 56 days. In some aspects, the composition, pharmaceutical composition, or vaccine is administered twice in an interval of about 7 days; about 10 days; about 14 days; about 21 days; about 28 days about 35 days or about 42 days. In some aspects, the composition, pharmaceutical composition, or vaccine is administered from about 2 times to about 20 times with intervals of about 1 week; 2 weeks; 3 weeks; 4 weeks; 5 weeks; 6 weeks; 7 weeks; 8 weeks; 9 weeks; 10 weeks; 11 weeks; 12 weeks; 13 weeks or about 14 weeks between each administration.

Certain aspects of the disclosure are directed to methods of making a vaccine, the method comprising the steps of: (a) combining the delivery component with the polynucleotide of the composition or pharmaceutical composition disclosed herein, (b) lyophilizing the combined delivery component and polynucleotide to a powder, and (c) reconstituting the powder with a diluent that comprises the adjuvant to form a vaccine solution.

### BRIEF DESCRIPTION OF THE DRAWINGS

FIG. 1 shows a schematic of a vector construct comprising polynucleotide sequences of a gene encoding a SARS-CoV-2 spike (S) protein under the control of promoter 1 (Prom-1), a gene encoding a second SARS-CoV-2 protein under the control of promoter 2 (Prom-2), and genes encoding interleukin 12 (IL-12) p35 and IL-12 p40 under the control of two cytomegalovirus (CMV) promoters in a plasmid backbone.

FIG. 2 shows a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), genes encoding IL-12 p35 and IL-12 p40 under the control of two CMV promoters, and a gene encoding major histocompatibility complex class I (MHC I) under the control of promoter Z (Prom-Z) in a plasmid backbone.

FIG. 3 shows a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), genes encoding IL-12 p35 and IL-12 p40 under the control of two CMV promoters, and a gene encoding major histocompatibility complex class II (MHC II) under the control of promoter Z (Prom-Z) in a plasmid backbone.

FIG. 4 shows a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), and a gene encoding interleukin 2 (IL-2) under the control of a CMV promoter in a plasmid backbone.

FIG. 5 shows a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding IL-2 under the control of a CMV promoter, and a gene encoding MHC I under the control of promoter Z (Prom-Z) in a plasmid backbone.

FIG. 6 shows a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding IL-2 under the control of a CMV promoter, and a gene encoding MHC II under the control of promoter Z (Prom-Z) in a plasmid backbone.

FIG. 7 shows a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding IL-2 under the control of a CMV promoter, a gene encoding C—C motif chemokine ligand (CCL) 3 (CCL3) under the control of promoter X (Prom-X), and a gene encoding CCL4 under the control of promoter Y (Prom-Y) in a plasmid backbone.

FIG. 8 shows a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), and a gene encoding interleukin 15 (IL-15) under the control of a CMV promoter in a plasmid backbone.

FIG. 9 shows a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding IL-15 under the control of a CMV promoter, and a gene encoding MHC I under the control of promoter Z (Prom-Z) in a plasmid backbone.

FIG. 10 shows a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding IL-15 under the control of a CMV promoter, and a gene encoding MHC II under the control of promoter Z (Prom-Z) in a plasmid backbone.

FIG. 11 shows a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding IL-15 under the control of a CMV promoter, a gene encoding CCL3 under the control of promoter X (Prom-X), and a gene encoding CCL4 under the control of promoter Y (Prom-Y) in a plasmid backbone.

FIG. 12 shows a schematic of a vector construct comprising polynucleotide sequences of a gene encoding a SARS-CoV-2 S protein under the control of promoter 1 (Prom-1), a gene encoding a second SARS-CoV-2 protein under the control of promoter 2 (Prom-2), a gene encoding CCL3 under the control of promoter X (Prom-X), and a gene encoding CCL4 under the control of promoter Y (Prom-Y) in a plasmid backbone.

FIG. 13A shows a schematic of the full-length SARS-CoV-2 S protein with S1 and S2 subunits demarcated. FIG. 13B shows pUNO (Invivogen) and constructs pVac1-pVac 5 (FIG. 13B).

FIGS. 14A-14AAK show schematics of vectors, including pVac vectors. Constructs express the following proteins: partial SARS-CoV-2 Spike Protein (S1, amino acids 15-695), complete SARS-CoV-2 Spike Protein (S, amino acids 1-1273) with a D614G amino acid substitution, SARS-CoV-2 Spike Protein with the D614G mutation and the 2P modification, SARS-CoV-2 Spike Protein with the D614G mutation and deletion of the last 17 amino acids of the C terminal domain (C-del), SARS-CoV-2 C-del Spike Protein with the 2P modification, full length SARS-CoV-2 delta variant Spike Protein, full length SARS-CoV-2 delta variant Spike Protein with the 2P modification, SARS-CoV-2 omicron variant Spike Protein, SARS-CoV-2 omicron variant Spike Protein with 2P modification, RSV F protein, CMV Gb Protein, H1N1 2007 Brisbane variant HA protein, SARS-CoV-2 omicron BA.2.75.2 variant Spike Protein, SARS-CoV-2 omicron 22E variant Spike Protein, SARS-CoV-2 beta variant Spike Protein, SARS-CoV-2 Nucleocapsid Protein, SARS-CoV-2 omicron BA.5 variant Spike Protein, SARS-CoV-2 omicron BQ.1 variant Nucleocapsid Protein, or SARS-CoV-2 omicron XBB1.5 variant Spike Protein. S proteins are under the control of a mammalian EF-1α or CMV promoter. FIG. 14A provides a pUNO vector comprising genes expressing the full-length not codon-optimized SARS-CoV-2 Spike Protein. FIG. 14B provides pVac1 expressing the SARS-CoV-2 Spike Protein S1 Subunit. FIG. 14C provides pVac2 expressing the SARS-CoV-2 Spike Protein S1 Subunit and IL-12p35 and IL-12 p40 under the control of two distinct CMV promoters. FIG. 14D provides pVac3 expressing the SARS-CoV-2 Spike Protein S1 Subunit, co-expressing the CoV-2 M antigen under the control of an Internal Ribosome Entry Site (IRES) sequence, the human heterodimeric cytokine IL12p35 and IL-12 p40 under the control of two distinct CMV promoters. FIG. 14E provides pVac4 expressing the full-length SARS-CoV-2 Spike Protein with D614G mutation. FIG. 14F provides pVac5 expressing the full-length SARS-CoV-2 Spike Protein with D614G mutation and co-expressing the human heterodimeric cytokine IL12 p35 and IL-12 p40 under the control of two distinct CMV promoters. FIG. 14G provides pVac6 expressing the full-length SARS-CoV-2 Spike Protein with D614G mutation with M antigen under the control of an Internal Ribosome Entry Site (IRES) sequence and co-expressing the human heterodimeric cytokine IL12p35 and IL-12 p40 under the control of two distinct CMV promoters. FIG. 14H provides pVac7 expressing the full-length SARS-CoV-2 Spike Protein with D614G mutation with M antigen under the control of a CMV promoter. FIG. 14I provides the p2CMV-V4 backbone vector used to construct the pVac 1 vector. FIG. 14J provides pVac8 expressing the SARS-CoV-2 Spike Protein with D614G mutation and deletion of the last 17 amino acids of the C terminal domain (C-del).

FIG. 14K provides pVac9 expressing the SARS-CoV-2 Spike Protein with D614G mutation and 2P modification at amino acids 986(K) and 987(V) (KV-PP). FIG. 14L provides pVac10 expressing the full length SARS-CoV-2 Spike Protein. FIG. 14M provides pVac11 expressing the full length SARS-CoV-2 delta variant Spike Protein under the control of a CMV promoter. FIG. 14N (FIG. 14N) provides pVac12 expressing the full length SARS-CoV-2 Spike Protein under the control of a CMV promoter and the SARS-CoV-2 full length delta variant Spike Protein under the control of a CMV promoter. FIG. 14O provides pVac13 expressing the full length SARS-CoV-2 Spike Protein with the aid of an SV40 Enhancer sequence. FIG. 14P provides pVac14 expressing the SARS-CoV-2 Spike Protein with the C terminal deletion and 2P modification. FIG. 14Q provides pVac15 expressing the full length SARS-CoV-2 Spike Protein with the 2P modification driven by a CMV promoter. FIG. 14R provides pVac16 expressing the full length SARS-CoV-2 delta variant Spike Protein with the 2P modification driven by a CMV promoter. FIG. 14S provides pVac17 expressing the full length SARS-CoV-2 Spike Protein with the 2P modification and expressing the SARS-CoV-2 full length delta variant Spike Protein with the 2P modification under the control of a CMV promoter. FIG. 14T provides pVac18 expressing the SARS-CoV-2 Spike Protein with the C terminal deletion and 2P modification and expressing the SARS-CoV-2 delta variant Spike Protein with the C terminal deletion and 2P modification under the control of an EF1-alpha promoter. FIG. 14U provides pVac19 expressing the SARS-CoV-2 Spike Protein with the C terminal deletion and 2P modification and expressing the SARS-CoV-2 delta variant Spike Protein with the C terminal deletion and 2P modification under the control of a CMV promoter. FIG. 14V provides pVac20 expressing the SARS-CoV-2 Spike Protein with the C terminal deletion and 2P modification driven by a CMV promoter. FIG. 14W provides pVac21 expressing the SARS-CoV-2 delta variant Spike Protein with the C terminal deletion and 2P modification driven by a CMV promoter. FIG. 14X provides pVac22 expressing the SARS-CoV-2 Spike Protein with the C terminal deletion driven by a CMV promoter.

FIG. 14Y provides pVac23 expressing the SARS-CoV-2 delta variant Spike Protein with the C terminal deletion driven by a CMV promoter. FIG. 14Z provides pVac24 expressing the SARS-CoV-2 Spike Protein with the C terminal deletion driven by a CMV promoter and expresses the SARS-CoV-2 delta variant Spike Protein with the C terminal deletion driven by a CMV promoter. FIG. 14AA provides pVac25 expressing the SARS-CoV-2 Spike Protein with the C terminal deletion and 2P modification driven by a CMV promoter and expresses the SARS-CoV-2 delta variant Spike Protein with the C terminal deletion and 2P modification driven by a CMV promoter. FIG. 14AB provides pVac26 expressing the SARS-CoV-2 delta variant Spike Protein with the C terminal deletion and 2P modification driven by an EF 1 promoter. FIG. 14AC provides pVac27 expressing the SARS-CoV-2 delta variant Spike Protein with the 2P modification driven by an EF 1 promoter. FIG. 14AD provides pVac28 expressing the SARS-CoV-2 Spike Protein with the 2P modification driven by an EF 1 promoter and expresses the SARS-CoV-2 delta variant Spike Protein with the 2P modification driven by an EF 1 promoter. FIG. 14AE provides pVac29 expressing the SARS-CoV-2 delta variant Spike Protein with the C terminal deletion driven by an EF1 promoter. FIG. 14AF provides pVac30 expressing the SARS-CoV-2 Spike Protein with the C terminal deletion driven by an EF 1 promoter and expresses the SARS-CoV-2 delta variant Spike Protein with the C terminal deletion driven by an EF 1 promoter. FIG. 14AG provides pVac31 expressing the SARS-CoV-2 Spike Protein with the 2P modification driven by a CMV promoter with the aid of an SV40 enhancer sequence. FIG. 14AH provides pVac32 expressing the SARS-CoV-2 delta variant Spike Protein with the 2P modification driven by a CMV promoter with the aid of an SV40 enhancer sequence. FIG. 14AI provides pVac33 expressing the SARS-CoV-2 Spike Protein with the 2P modification driven by a CMV promoter with the aid of an SV40 enhancer sequence and expressing the SARS-CoV-2 delta variant Spike Protein with the 2P modification driven by a CMV promoter with the aid of an SV40 enhancer sequence. FIG. 14AJ provides pVac34 expressing the SARS-CoV-2 Omicron variant Spike Protein driven by a CMV promoter with the aid of an SV40 enhancer sequence.

FIG. 14AK provides pVac35 expressing the SARS-CoV-2 Omicron variant Spike Protein with the 2P modification driven by a CMV promoter with the aid of an SV40 enhancer sequence. FIG. 14AL (FIG. 14AL) provides pVac36 expressing the SARS-CoV-2 Spike Protein with the 2P modification driven by a CMV promoter with the aid of an SV40 enhancer sequence and expressing the SARS-CoV-2 Omicron variant Spike Protein with the 2P modification driven by a CMV promoter with the aid of an SV40 enhancer sequence. FIG. 14AM provides pVac37 expressing the RSV F Protein driven by a CMV promoter with the aid of an SV40 enhancer sequence. FIG. 14AN provides pVac38 expressing the CMV Gb Protein driven by a CMV promoter with the aid of an SV40 enhancer sequence. FIG. 14AO provides pH1N1 expressing the H1N1 2007 Brisbane variant HA protein driven by a CMV promoter. FIG. 14AP provides pVac40 expressing the SARS-CoV-2 Spike Protein with D614G mutation and the 2P modification driven by a CMV promoter with the aid of a SV40 enhancer sequence and expressing the luciferase protein driven by a CMV promoter with the aid of a SV40 enhancer sequence. FIG. 14AQ provides pVac42 expressing the SARS-CoV-2 Spike Protein with D614G mutation and the 2P modification driven by a CMV promoter. FIG. 14AR provides pVac43 expressing SARS-CoV-2 Spike Protein with D614G mutation and the 2P modification driven by a CMV promoter and expressing the full length SARS-CoV-2 delta variant Spike Protein with the 2P modification driven by a CMV promoter. FIG. 14AS provides pVac44 expressing SARS-CoV-2 Spike Protein with D614G mutation and the 2P modification driven by a CMV promoter with the CMV intron A. FIG. AT provides pVac45 expressing SARS-CoV-2 Spike Protein with D614G mutation and the 2P modification driven by a CMV promoter with the aid of a SV40 enhancer sequence and expressing the full length SARS-CoV-2 delta variant Spike Protein with the 2P modification driven by a CMV promoter with the aid of a SV40 enhancer sequence. FIG. 14AU provides pVac46 expressing SARS-CoV-2 Spike Protein with D614G mutation and the 2P modification driven by a CMV promoter with a CMV intron A and the aid of a SV40 enhancer sequence and expressing the full length SARS-CoV-2 delta variant Spike Protein with the 2P modification driven by a CMV promoter with a CMV intron A and the aid of a SV40 enhancer sequence. FIG. 14V provides pVac47 expressing the SARS-CoV-2 Spike Protein with D614G mutation and the 2P modification driven by a CMV promoter with a CMV intron A and the aid of a SV40 enhancer sequence. FIG. 14W provides pVac48 expressing the full length SARS-CoV-2 delta variant Spike Protein with the 2P modification driven by a CMV promoter with a CMV intron A and the aid of a SV40 enhancer sequence. FIG. 14AX provides pVac49 expressing SARS-CoV-2 Spike Protein with D614G mutation and the 2P modification driven by a CMV promoter with a CMV intron A and the aid of a SV40 enhancer sequence and expressing the full length SARS-CoV-2 delta variant Spike Protein with the 2P modification driven by a CMV promoter with a CMV intron A and a SV40 enhancer sequence. FIG. 14AY provides pVac50 expressing the full length SARS-CoV-2 delta variant Spike Protein with the 2P modification driven by a CMV promoter with the aid of an SV40 enhancer sequence. FIG. 14AZ provides pVac51 expressing the SARS-CoV-2 Omicron BA.2.75.2 variant Spike Protein driven by a CMV promoter. FIG. 14AAA provides pVac52 expressing the SARS-CoV-2 BA.2.75.2 variant Spike Protein with a IgE leader sequence and driven by a CMV promoter. FIG. 14AAB provides pVac53 expressing the full length SARS-CoV-2 delta variant Spike Protein with the 2P modification driven by a CMV promoter with the aid of an SV40 enhancer sequence. FIG. 14AAC provides pVac54 expressing SARS-CoV-2 Spike Protein with D614G mutation and the 2P modification driven by a CMV promoter with the aid of a SV40 enhancer sequence and expressing the full length SARS-CoV-2 delta variant Spike Protein with the 2P modification driven by the CMV promoter with the aid of a SV40 enhancer sequence. FIG. 14AAD provides pVac55 expressing the SARS-CoV-2 Omicron 22E variant Spike Protein with the 2P modification driven by a CMV promoter with the aid of a SV40 enhancer sequence. FIG. 14AAE provides pVac56 expressing the SARS-CoV-2 Omicron 22E variant Spike Protein with the 2P modification and furin cleavage mutation driven by the CAG promoter with the aid of a SV40 enhancer sequence. FIG. 14AAF provides pVac57 expressing the SARS-CoV-2 Beta variant Spike Protein with the 2P modification and furin cleavage mutation driven by a CMV promoter with the aid of a SV40 enhancer sequence. FIG. 14AAG provides pVac58 expressing SARS-CoV-2 Nucleocapsid Protein driven by a CMV promoter with the aid of a SV40 enhancer sequence. FIG. AAH provides pVac59 expressing the SARS-CoV-2 Omicron 22E (BQ.1) variant Spike Protein with the 2P modification and furin cleavage mutation driven by the CMV promoter with the aid of the SV40 enhancer sequence and expressing the SARS-CoV-2 Beta variant Spike Protein with the 2P modification and furin cleavage mutation driven by the CMV promoter with the aid of the SV40 enhancer sequence. FIG. 14AAI provides pVac60 expressing the SARS-CoV-2 Omicron 22E (BQ.1) variant Spike Protein with the 2P modification and furin cleavage mutation driven by the CAG promoter with the aid of a SV40 enhancer sequence and expressing the SARS-CoV-2 Beta variant Spike Protein with the 2P modification and furin cleavage mutation driven by a CMV promoter with the aid of a SV40 enhancer sequence. FIG. 14AAJ provides pVac61 expressing the SARS-CoV-2 Omicron BA.5 variant Spike Protein driven by a CMV promoter with the aid of a SV40 enhancer sequence, expressing the SARS-CoV-2 Nucleocapsid Protein driven by a CMV promoter with the aid of a SV40 enhancer sequence, and expressing SARS-CoV-2 Spike Protein with D614G mutation and the 2P modification driven by a CMV promoter with the aid of a SV40 enhancer sequence. FIG. 14AAK provides pVac62 expressing the SARS-CoV-2 Omicron BQ.1 variant Nucleocapsid Protein driven by a CMV promoter with the aid of a SV40 enhancer sequence and expressing the SARS-CoV-2 Omicron XBB1.5 Spike Protein driven by a CMV promoter with the aid of a SV40 enhancer sequence. FIG. 15 (FIG. 15) shows a schematic of in vivo studies in BALB/c mice immunized with various doses of vaccine (1 ug-500 g) with gaps between vaccinations of 2 weeks, 3 weeks, or 4 weeks in the absence of a booster or with one or two booster immunizations.

FIG. 16A (FIG. 16A) shows the IgG titers in BALB/c mice immunized with 125 μg of pVac-15, pVac-16, or pVac-17, in combination with crown poloxamer, and aluminum PO₄ adjuvant administered i.m. on days 0, 14 and 35; PBS as negative control and SARS-CoV-2D614-2P mRNA as positive control.

FIG. 16B (FIG. 16B) shows the virus neutralizing activity of a multi-antigen single plasmid vaccine in a pseudoviral challenge assay from BALB/c mice immunized with 125 g of pVac-17 or SARS-CoV-2 D614-2P mRNA control in combination with crown poloxamer and aluminum P04 adjuvant administered i.m. at days 0, 14 and 35.

FIG. 16C (FIG. 16C) shows the virus neutralizing activity of two single antigen plasmid vaccines in a pseudoviral challenge assay from BALB/c mice immunized with 125 g of pVac-15 or pVac-16 in combination with crown poloxamer and aluminum P04 adjuvant administered i.m. at days 0, 14 and 35.

FIG. 17A (FIG. 17A) shows the IFN-7 production as SFC per 1 Mil. spleen cells isolated from BALB/c mice 35 days after immunization with 125 g of pVac-9 with crown poloxamer, crown poloxamer and STING agonist, or crown poloxamer and aluminum PO₄, or PBS control.

FIG. 17B (FIG. 17B) shows the IgG titers of BALB/c mice 35 days after immunization with 125 g pVac-9 with crown poloxamer, crown poloxamer and STING agonist, or crown poloxamer and aluminum P04 or PBS control.

FIG. 18A (FIG. 18A) shows the IgG titers of BALB/c mice 14 and 35 days after i.m. immunization on days 0, 14, and 35 with 250 g of pVac-9 in PBS or pVac-9 with crown poloxamer.

FIG. 18B (FIG. 18B) shows the IFN-7 production as SFC per 1 Mio. cells isolated from BALB/c mice 35 days after immunization on days 0, 14, and 35 with 250 g of pVac-9 in PBS or pVac-9 with crown poloxamer.

FIG. 19 (FIG. 19) shows the IgG titers of BALB/c mice 35 days after immunization with 125 g pH1N1 Brisbane 2007 with crown poloxamer, or crown poloxamer and aluminum P04 or PBS control.

FIG. 20 (FIG. 20) shows the percent starting body weight of BALB/c mice immunized with pUNO Spike vector (C2) alone or in combination with 0.5% crown poloxamer (C2/F1), crown poloxamer and 10 μg STING agonist (C2/F2), or 0.5% crown poloxamer and 50 μg aluminum P04 (C2/F3) during the first three days after immunization and after the first booster immunization (days 14-16). The percent weight of BALB/c mice immunized with 10⁸ viral particles (VIRAL), 10 g of spike protein (PROTEIN) or 1 g of a SARS-Cov-2 RNA with a K986P and V987P mutation (RNA/2P) are shown.

FIG. 21 shows the IgG titers of Balb/C mice 35 days after immunization with 125 μg plasmid DNA of pVac42 or pVac31 and 0.5% crown poloxamer (CP) with or without 0.5 mg/mL aluminum P04 (AIPO4) or PBS control.

FIG. 22 shows the virus neutralizing activity of two single antigen plasmid vaccines in a pseudoviral challenge assay from Balb/C mice 35 days after immunization with 125 μg plasmid DNA of pVac31 or pVac42 and 0.5% crown poloxamer (CP) with or without 0.5 mg/mL aluminum P04 adjuvant (AIP04).

FIG. 23 shows the IgG titers of Balb/C mice 35 days after immunization with 125 μg plasmid DNA with pVac16 with 0.5% crown poloxamer (CP), or pVac16 with 0.5% crown poloxamer and 0.5 mg/mL 0.5 mg/mL aluminum P04 (AIP04), or PBS control.

FIG. 24 shows the virus neutralizing activity of a single antigen plasmid vaccine in a pseudoviral challenge assay from BALB/c mice 35 days after immunization with pVac16 with 0.5% crown poloxamer (CP) or pVac16 with 0.5% crown poloxamer and 0.5 mg/mL aluminum P04 adjuvant (AIPO4).

FIG. 25 shows the IFN-7 production as SFC per 1 Mil. spleen cells isolated from BALB/c mice after immunization with 125 g of pUNO with 0.5% crown poloxamer (CP), 125 g of pUNO with 0.5% crown poloxamer (CP) and STING agonist, or PBS control.

FIG. 26 shows the IFN-7 production as SFC per 1 Mil. spleen cells isolated from BALB/c mice after immunization with 125 g of pVac17 with 0.5% crown poloxamer (CP), 125 g of pVac17 with 0.5% crown poloxamer and STING agonist, or PBS control.

FIG. 27A shows a schematic of in vivo studies in hACE2:K18 mice immunized with 125 μg pVac15, pVac16, or pVac17 day 0 with a booster at day 14.

FIG. 27B shows the tissue culture infectious dose (TCID₅₀) of single antigen plasmid vaccines or a multi-antigen plasmid vaccine isolated from the lung of hACE2:K18 mice 7 days after challenge with D614G strain that were previously immunized with placebo, pVac15 with 0.5% crown poloxamer and 0.5 mg/mL aluminum P04 adjuvant, or pVac17 with 0.5% crown poloxamer and 0.5 mg/mL aluminum P04 adjuvant.

FIG. 27C shows the tissue culture infectious dose (TCID₅₀) of single antigen plasmid vaccines or a multi-antigen plasmid vaccine isolated from the lung of hACE2:K18 mice 7 days after challenge with the Delta strain that were previously immunized with placebo, pVac16 with 0.5% crown poloxamer and 0.5 mg/mL aluminum P04 adjuvant, or pVac17 with 0.5% crown poloxamer and 0.5 mg/mL aluminum P04 adjuvant.

FIG. 28 shows the IgG titers of Cynomolgus monkeys after immunization with placebo, 1 mg pVac15 with 0.5% crown poloxamer (CP) and 0.5 mg/mL aluminum P04 adjuvant, 2 mg pVac16 with 0.5% crown poloxamer (CP) and 0.5 mg/mL aluminum P04 adjuvant, 5 mg pVac15 and 1 mg pVac16 with 0.5% crown poloxamer (CP) and 0.5 mg/mL aluminum P04 adjuvant, or 100 g mRNA vaccine.

FIG. 29 shows the virus neutralizing titers from Cynomolgus monkeys after immunization with placebo, 1 mg pVac15 with 0.5% crown poloxamer (CP) and 0.5 mg/mL aluminum P04 adjuvant, 2 mg pVac16 with 0.5% crown poloxamer (CP) and 0.5 mg/mL aluminum P04 adjuvant, 5 mg pVac15 and 1 mg pVac16 with 0.5% crown poloxamer (CP) and 0.5 mg/mL aluminum PO₄ adjuvant, or 100 g mRNA vaccine.

FIG. 30A shows the amount viral mRNA isolated from the lung of Cynomolgus monkeys immunized with placebo, pVac15 with 0.5% crown poloxamer (CP) and 0.5 mg/mL aluminum PO₄ adjuvant, pVac16 with 0.5% crown poloxamer (CP) and 0.5 mg/mL aluminum PO₄ adjuvant, or control mRNA vaccine 2, 4, and 7 days after viral challenge.

FIG. 30B shows the amount viral mRNA isolated from the nasal passage of Cynomolgus monkeys immunized with placebo, pVac15 with 0.5% crown poloxamer (CP) and 0.5 mg/mL aluminum PO₄ adjuvant, pVac16 with 0.5% crown poloxamer (CP) and 0.5 mg/mL aluminum PO₄ adjuvant, or control mRNA vaccine 2, 4, and 7 days after viral challenge.

FIG. 31A shows the virus neutralizing activity of vaccines in a pseudoviral challenge assay from mice 1, 2, 3, 5, 6, or 8 months after immunization with 125 g of pVac16 with 0.5% crown poloxamer (CP) and 0.5 mg/mL aluminum PO₄ adjuvant, pVac17 with 0.5% crown poloxamer (CP) and 0.5 mg/mL aluminum PO₄ adjuvant, or control mRNA vaccine.

FIG. 31B shows the IFN-7 production as SFC per 1 Mil. spleen cells isolated from mice 12 months after immunization with 125 g of pVac15 with 0.5% crown poloxamer (CP) and 0.5 mg/mL aluminum PO₄ adjuvant, 125 g of pVac17 with 0.5% crown poloxamer and 0.5 mg/mL aluminum PO₄ adjuvant, control mRNA vaccine, or PBS control.

FIG. 32A shows the IFN-7 production as SFC per 1 Mil. spleen cells isolated from mice administered 125 g pVac17 with 0.5% crown poloxamer and 0.5 mg/mL aluminum P04 adjuvant or 125 g pVac54 with 0.5% crown poloxamer and 0.5 mg/mL aluminum PO₄ adjuvant.

FIG. 32B shows the percent of IFN-7 and TNF-α expressing CD8+ T cells in spleenocytes in mice after immunization with 125 g of pVac17 with 0.5% crown poloxamer and 0.5 mg/mL aluminum PO₄ adjuvant, 125 g of pVac54 with 0.5% crown poloxamer and 0.5 mg/mL aluminum P04 adjuvant, or PBS control.

FIG. 33A shows the IgG titers of mice 2, 4, or 5 weeks after immunization with a single dose of pVac50 with 0.5% crown poloxamer and 0.5 mg/mL aluminum P04 adjuvant, control mRNA vaccine, or PBS control.

FIG. 33B shows the IFN-7 production as SFC per 1 Mil. spleen cells isolated from mice after immunization with a single dose of pVac50 with 0.5% crown poloxamer and 0.5 mg/mL aluminum PO₄ adjuvant, control mRNA vaccine, or PBS control.

FIG. 34 shows the IgG titers of mice after immunization with freshly prepared pVac17 or pVac17 that had been stored at 4° C. for at least 9 months.

### DETAILED DESCRIPTION OF THE DISCLOSURE

### I. Overview

Provided herein are compositions, including pharmaceutical compositions and vaccines, that comprise polynucleotides (e.g., DNA or mRNA), vectors (e.g., expression vectors), multicistronic mRNA vectors, and DNA plasmid vectors of the disclosure to address, for example, the issue of suboptimal immunogenicity often associated with DNA-based vaccine approaches on one or more levels. First, in some aspects, the DNA-based vaccine compositions can further comprise an adjuvant or combination of adjuvants, e.g., an aluminum or aluminum-salt based adjuvant and stimulator of interferon genes (STING) agonist. Second, in some aspects, the DNA-based vaccine composition can further comprise polynucleotides that co-express one or more immune modifier proteins such as cytokines and/or chemokines that augment the immune responses to the viral antigen(s). Third, in some aspects, the polynucleotides of the DNA-based vaccine can include multiple viral antigens and/or multiple epitomes of a viral antigen instead of a single viral antigen, which can be co-expressed from the vectors disclosed herein to expand the spectrum of immunogenicity. Fourth, in some aspects, to improve vaccine efficiency, the expression vectors (e.g., DNA plasmid vectors) disclosed herein can be formulated with delivery systems (e.g., a cationic polymer, a poly-inosinic-polycylidylic acid, a poloxamer, or a derivative thereof) that protects the vector or polynucleotide from nuclease degradation and promote its translocation through cell compartments. In some aspects, the delivery systems disclosed herein can also be formulated to exhibit an additional adjuvant property to promote mobilization of antigen presenting cells to the site of vaccine delivery and antigen expression, thereby augmenting the uptake of the vaccine vector and the expressed viral antigens into professional antigen presenting cells to elicit MHC Class I and MHC Class II presentation. See Greenland, J. R., et al., Molecular Therapy, 12(1):164-70 (2005) (hereafter “Greenland 2005”) and Suschak, J. J., et al., Human Vaccines & Immunotherapeutics, 13(12):2837-48 (2017) (hereafter “Suschak 2017”).

In some aspects, the DNA-based vaccine compositions comprise an ALUM salt adjuvant. In some aspects, the ALUM salt is selected from aluminum phosphate, aluminum hydroxide, potassium aluminum sulfate [KAl(SO₄)₂], aluminumcrystalline aluminum oxyhydroxide, aluminum hydroxyphosphate, amorphous aluminum hydroxyphosphate sulfate, aluminum chloride, aluminum silicate, and a mixture of aluminum hydroxide and magnesium hydroxide, a mixture of aluminum sulfate and sodium hydroxide or a mixture of aluminum sulfate and potassium hydroxide.

In some aspects, the DNA-based vaccine composition comprise a STING agonist. In some aspects, the STING agonist is selected from a cyclic di-nucleotides, a non-cyclic di-nucleotide small molecule, an amidobenzimidazole, a nanovaccine, an antibody drug conjugate, a bacterial vector, and an ENPP1 inhibitor.

In some aspects, the STING agonist is a cyclic dinucleotide cGMP, cAMP, or cGMP-AMP. In some aspects, the STING agonist is cGMP.

In some aspects, the STING agonist is a cyclic dinucleotide selected from ADU-S100, MK-1454, SB11285, BMS-986301, BI-STING (BI1387446), JNJ-67544412, 3′3′-cyclic AIMP, and GSK532.

In some aspects, the STING agonist is a non-cyclic dinucleotide small molecule selected from DMAXAA, ALG-031048, E7766, JNJ-′6196, MK-21118, MSA-1, MSA-2, SNX281, SR-717, TAK676, TTI-10001, a Ryvu's agonist, GF3-002, a Selvita agonist, CDR5500, CS-1010, CS-1018, CS-1020, and [2-ex] MSA-1.

In some aspects, the STING agonist is a nanovaccine selected from PC7A nanoparticles, cGMP-nanoparticles, and ONM-500 nanoparticles.

In some aspects, the STING agonist is an antibody-drug conjugate. In some aspects, the antibody drug conjugate is XMT-2056. In some aspects, the STING agonist is an ENPP1 inhibitor selected from MV-626, SR-8314, SR-8291, and SR8541A.

In some aspects, the STING agonist is a bacterial vector. In some aspects, the bacterial vector is a nonpathogenic E. coli nissle expressing cyclic-di-AMP-producing enzymes. In some aspects, the bacterial vector is SYNB1981. In some aspect, the bacterial vector is an attenuated Salmonella Typhimurium strain engineered to carry an inhibitory TREX-1 micro RNA.

In some aspects, the polynucleotide (e.g., expression vector) can comprise a nucleic acid sequence encoding one or more viral antigens (e.g., a SARS CoV-2 antigen). In some aspects, the one or more viral antigens comprise viral antigens (e.g., a S protein, a S1 subunit of a S protein, a RBD of a S protein, a membrane fusion domain of a S protein, a M protein, an E protein, or an antigenic fragment thereof) from two or more SARS-CoV-2 strains. In some aspects, the polynucleotide (e.g., expression vector) further comprises a nucleic acid sequence encoding one or more immune modifier proteins. In some aspects, the polynucleotide (e.g., expression vector) comprises a nucleic acid sequence encoding a SARS CoV-2 antigen and, optionally, a second viral antigen. In some aspects, the polynucleotide (e.g., expression vector) comprises a nucleic acid sequence encoding a SARS CoV-2 antigen and a second SARS CoV-2 antigen from a different SARS CoV-2 strain. In some aspects, the polynucleotide (e.g., expression vector) comprises a nucleic acid sequence encoding a SARS CoV-2 S protein antigen and a second SARS CoV-2 S protein antigen from a different SARS CoV-2 strain.

In some aspects, the polynucleotide (e.g., expression vector) comprises a nucleic acid sequence encoding a SARS CoV-2 antigen, a second viral antigen, and a third viral antigen. In some aspects, the polynucleotide (e.g., expression vector) comprises a nucleic acid sequence encoding a SARS CoV-2 antigen and a second SARS CoV-2 antigen from a different SARS CoV-2 strain. In some aspects, the polynucleotide (e.g., expression vector) comprises a nucleic acid sequence encoding a SARS CoV-2 S protein antigen and a second SARS CoV-2 S protein antigen from a different SARS CoV-2 strain.

In some aspects, the polynucleotide (e.g., expression vector) comprises a nucleic acid sequence encoding a SARS CoV-2 antigen, a second SARS CoV-2 antigen, and a third SARS CoV-2 antigen from a SARS CoV-2 strains. In some aspects, the polynucleotide (e.g., expression vector) comprises a nucleic acid sequence encoding a SARS CoV-2 S protein antigen and a second SARS CoV-2 S protein antigen from a different SARS CoV-2 strain.

In some aspects, the present disclosure is directed to a composition comprising (a) a polynucleotide (e.g., an expression vector) comprising a single antigen nucleic acid which encodes a single pathogen antigen (e.g., a SARS-CoV-2 S protein) or an antigenic fragment thereof); and (b) an adjuvant comprising an aluminum or aluminum-salt based adjuvant and/or a STING agonist. In some aspects, the antigen nucleic acid of the polynucleotide is operably linked to a promoter. In some aspects, the polynucleotide further comprises one or more nucleic acids encoding an immune modifier protein.

In some aspects, the present disclosure is directed to a composition comprising (a) a polynucleotide (e.g., a multicistronic DNA plasmid or multicistronic mRNA) comprising (i) an antigen nucleic acid which encodes a pathogen antigen (e.g., a SARS-CoV-2 S protein, a monkeypox A35R protein, a monkeypox H3L protein, a monkeypox L1R protein, or an antigenic fragment thereof) and (ii) at least one additional antigen nucleic acid which encodes an additional pathogen antigen and/or a nucleic acids encoding an immune modifier protein; and (b) an adjuvant comprising an aluminum or aluminum-salt based adjuvant and/or a STING agonist. In some aspects, the antigen nucleic acid (e.g., first antigen nucleic acid) of the polynucleotide is operably linked to a promoter. In some aspects, the polynucleotide comprises one or more nucleic acids encoding an immune modifier protein.

In some aspects, the polynucleotide (e.g., an expression vector) comprises one or more nucleic acids encoding pathogen proteins or antigenic fragments thereof. In some aspects, the polynucleotide comprises at least two nucleic acids encoding different pathogen or antigenic fragments thereof. In some aspects, the polynucleotide comprises one, two, three, four, five six, seven, or eight pathogen proteins or antigenic fragments thereof.

In some aspects, the polynucleotide (e.g., an expression vector) comprises at least one additional antigen nucleic acid (e.g., second antigen nucleic acid) which encodes at least one additional pathogen antigen (e.g., a second SARS-CoV-2 protein antigen or an antigenic fragment thereof). In some aspects, the polynucleotide (e.g., an expression vector) comprises at least two additional antigen nucleic acids (e.g., a second antigen nucleic acid and a third antigen nucleic acid) which encodes at least two additional pathogen antigens (e.g., a second SARS-CoV-2 protein antigen or an antigenic fragment thereof and a third SARS-CoV-2 protein antigen or an antigenic fragment thereof).

As described herein, the nucleic acid molecules of the present disclosure comprise one or more features that distinguish the present nucleic acid molecules form those that exist in nature (e.g., comprising at least one gene encoding a SARS-CoV-2 S protein and a heterologous promoter). Not to be bound by any theory, in some aspects, the expression of multiple pathogen antigens (e.g., SARS-CoV-2 antigens or monkeypox antigens), expands the spectrum of immunogenicity, while the presence of an adjuvant (e.g., a STING agonist in the composition) and optionally the expression of at least one immune modifier protein augment the immune responses to the multiple pathogen antigens. In some aspects, the pathogen antigens are viral pathogen antigens, bacterial pathogen antigens, or parasite pathogen antigens.

In some aspects, the composition comprises (i) a polynucleotide (e.g., an expression vector) comprising: a nucleic acid encoding a pathogen antigen (e.g., SARS-CoV-2 spike (S) protein, a monkeypox A35R protein, a monkeypox H3L protein, a monkeypox L1R protein, or an antigenic fragment thereof), wherein the nucleic acid is operably linked to a promoter and (ii) and adjuvant comprising an aluminum or aluminum-salt based adjuvant and/or a STING agonist.

In some aspects, the composition comprises (i) a polynucleotide (e.g., an expression vector) comprising: (a) a first nucleic acid encoding a first pathogen antigen (e.g., SARS-CoV-2 spike (S) protein, a monkeypox A35R protein, a monkeypox H3L protein, a monkeypox L1R protein, or an antigenic fragment thereof), wherein the first nucleic acid is operably linked to a first promoter; (b) a second nucleic acid encoding a second pathogen antigen (e.g., SARS-CoV-2 protein, a monkeypox protein, or an antigenic fragment thereof), wherein the second nucleic acid is operably linked to a second promoter; (c) a third nucleic acid encoding a third pathogen antigen (e.g., SARS-CoV-2 protein, a monkeypox protein, or an antigenic fragment thereof), wherein the third nucleic acid is operably linked to a third promoter; and (ii) an adjuvant comprising an aluminum or aluminum-salt based adjuvant and/or a STING agonist. In aspects, the composition comprises (i) a polynucleotide (e.g., an expression vector) comprising: (a) a first nucleic acid encoding a first pathogen antigen (e.g., SARS-CoV-2 spike (S) protein, a monkeypox A35R protein, a monkeypox H3L protein, a monkeypox L1R protein, or an antigenic fragment thereof), wherein the first nucleic acid is operably linked to a first promoter; (b) a second nucleic acid encoding a second pathogen antigen (e.g., SARS-CoV-2 protein, a monkeypox protein, or an antigenic fragment thereof), wherein the second nucleic acid is operably linked to a second promoter; (c) a third nucleic acid encoding a third pathogen antigen (e.g., SARS-CoV-2 protein, a monkeypox protein, or an antigenic fragment thereof), wherein the third nucleic acid is operably linked to a third promoter; (ii) an adjuvant comprising an aluminum or aluminum-salt based adjuvant and/or a STING agonist; and (iii) a delivery component (e.g., a cationic polymer, a poly-inosinic-polycytidylic acid, or a poloxamer).

In some aspects, the composition comprises (i) a polynucleotide (e.g., an expression vector) comprising: (a) a first nucleic acid encoding a first pathogen antigen (e.g., SARS-CoV-2 spike (S) protein, a monkeypox A35R protein, a monkeypox H3L protein, a monkeypox L1R protein, or an antigenic fragment thereof), wherein the first nucleic acid is operably linked to a first promoter; (b) a second nucleic acid encoding an immune modifier protein, wherein the second nucleic acid is operably linked to a second promoter; and (ii) an adjuvant comprising an aluminum or aluminum-salt based adjuvant and/or a STING agonist. In some aspects, the composition comprises (i) a polynucleotide (e.g., an expression vector) comprising: (a) a first nucleic acid encoding a first pathogen antigen (e.g., SARS-CoV-2 spike (S) protein, a monkeypox A35R protein, a monkeypox H3L protein, a monkeypox L1R protein, or an antigenic fragment thereof), wherein the first nucleic acid is operably linked to a first promoter; (b) a second nucleic acid encoding an immune modifier protein, wherein the second nucleic acid is operably linked to a second promoter; (ii) an adjuvant comprising an aluminum or aluminum-salt based adjuvant and/or a STING agonist; and (iii) a delivery component (e.g., a cationic polymer, a poly-inosinic-polycytidylic acid, or a poloxamer).

In some aspects, the composition comprises (i) a polynucleotide (e.g., an expression vector) comprising: (a) a first nucleic acid encoding a first pathogen antigen (e.g., SARS-CoV-2 spike (S) protein, a monkeypox A35R protein, a monkeypox H3L protein, a monkeypox L1R protein, or an antigenic fragment thereof), wherein the first nucleic acid is operably linked to a first promoter; (b) a second nucleic acid encoding a second pathogen antigen (e.g., SARS-CoV-2 spike (S) protein, a monkeypox A35R protein, a monkeypox H3L protein, a monkeypox L1R protein, or an antigenic fragment thereof), wherein the second nucleic acid is operably linked to a second promoter; (c) a third nucleic acid encoding an immune modifier protein, wherein the third nucleic acid is operably linked to a third promoter; and (ii) an adjuvant comprising an aluminum or aluminum-salt based adjuvant and/or a STING agonist.

In some aspects, the composition comprises (i) a polynucleotide (e.g., an expression vector) comprising: (a) a first nucleic acid encoding a first pathogen antigen (e.g., SARS-CoV-2 spike (S) protein, a monkeypox A35R protein, a monkeypox H3L protein, a monkeypox L1R protein, or an antigenic fragment thereof), wherein the first nucleic acid is operably linked to a first promoter; (b) a second nucleic acid encoding a second pathogen antigen (e.g., SARS-CoV-2 spike (S) protein, a monkeypox A35R protein, a monkeypox H3L protein, a monkeypox L1R protein, or an antigenic fragment thereof), wherein the second nucleic acid is operably linked to a second promoter; (c) a third nucleic acid encoding an immune modifier protein, wherein the third nucleic acid is operably linked to a third promoter; (ii) an adjuvant comprising an aluminum or aluminum-salt based adjuvant and/or a STING agonist; and (iii) a delivery component (e.g., a cationic polymer, a poly-inosinic-polycytidylic acid, or a poloxamer).

In some aspects, the composition comprises (i) a polynucleotide (e.g., an expression vector) comprising: (a) a first nucleic acid encoding a first pathogen antigen (e.g., SARS-CoV-2 spike (S) protein, a monkeypox A35R protein, a monkeypox H3L protein, a monkeypox L1R protein, or an antigenic fragment thereof), wherein the first nucleic acid is operably linked to a first promoter; (b) a second nucleic acid encoding a second pathogen antigen (e.g., SARS-CoV-2 spike (S) protein, a monkeypox A35R protein, a monkeypox H3L protein, a monkeypox L1R protein, or an antigenic fragment thereof), wherein the second nucleic acid is operably linked to a second promoter; (c) a third nucleic acid encoding a third pathogen antigen (e.g., SARS-CoV-2 spike (S) protein, a monkeypox A35R protein, a monkeypox H3L protein, a monkeypox L1R protein, or an antigenic fragment thereof), wherein the third nucleic acid is operably linked to a third promoter; (d) a fourth nucleic acid encoding an immune modifier protein, wherein the fourth nucleic acid is operably linked to a fourth promoter; and (ii) an adjuvant comprising an aluminum or aluminum-salt based adjuvant and/or a STING agonist.

In some aspects, the composition comprises (i) a polynucleotide (e.g., an expression vector) comprising: (a) a first nucleic acid encoding a first pathogen antigen (e.g., SARS-CoV-2 spike (S) protein, a monkeypox A35R protein, a monkeypox H3L protein, a monkeypox L1R protein, or an antigenic fragment thereof), wherein the first nucleic acid is operably linked to a first promoter; (b) a second nucleic acid encoding a second pathogen antigen (e.g., SARS-CoV-2 spike (S) protein, a monkeypox A35R protein, a monkeypox H3L protein, a monkeypox L1R protein, or an antigenic fragment thereof), wherein the second nucleic acid is operably linked to a second promoter; (c) a third nucleic acid encoding a third pathogen antigen (e.g., SARS-CoV-2 spike (S) protein, a monkeypox A35R protein, a monkeypox H3L protein, a monkeypox L1R protein, or an antigenic fragment thereof), wherein the third nucleic acid is operably linked to a third promoter; (d) a fourth nucleic acid encoding an immune modifier protein, wherein the fourth nucleic acid is operably linked to a fourth promoter; (ii) an adjuvant comprising an aluminum or aluminum-salt based adjuvant and/or a STING agonist; and (iii) a delivery component (e.g., a cationic polymer, a poly-inosinic-polycytidylic acid, or a poloxamer).

In some aspects, the composition comprises (i) a polynucleotide (e.g., an expression vector) comprising: (a) a first nucleic acid encoding a first pathogen antigen (e.g., SARS-CoV-2 spike (S) protein, a monkeypox A35R protein, a monkeypox H3L protein, a monkeypox L1R protein, or an antigenic fragment thereof), wherein the first nucleic acid is operably linked to a first promoter; (b) a second nucleic acid encoding a second pathogen antigen (e.g., SARS-CoV-2 protein, a monkeypox protein, or an antigenic fragment thereof), wherein the second nucleic acid is operably linked to a second promoter; and optionally (c) a third nucleic acid encoding an immune modifier protein, wherein the third nucleic acid is operably linked to a third promoter; and (ii) an adjuvant comprising an aluminum or aluminum-salt based adjuvant and/or a STING agonist.

In some aspects, the composition comprises (i) a polynucleotide (e.g., an expression vector) comprising: (a) a first nucleic acid encoding a first pathogen antigen (e.g., SARS-CoV-2 spike (S) protein, a monkeypox A35R protein, a monkeypox H3L protein, a monkeypox L1R protein, or an antigenic fragment thereof), wherein the first nucleic acid is operably linked to a first promoter; (b) a second nucleic acid encoding a second pathogen antigen (e.g., SARS-CoV-2 protein, a monkeypox protein, or an antigenic fragment thereof), wherein the second nucleic acid is operably linked to a second promoter; and optionally (c) a third nucleic acid encoding an immune modifier protein, wherein the third nucleic acid is operably linked to a third promoter; (ii) an adjuvant comprising an aluminum or aluminum-salt based adjuvant and/or a STING agonist; and (iii) a delivery component (e.g., a cationic polymer, a poly-inosinic-polycytidylic acid, or a poloxamer).

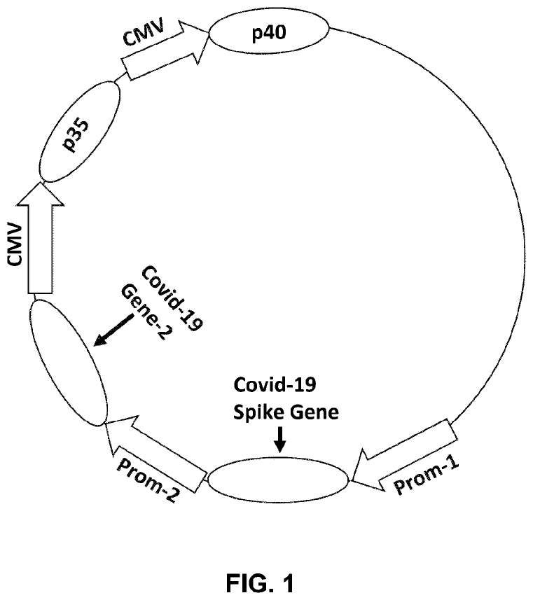

In some aspects, the polynucleotide can include the elements as disclosed in any of FIGS. 1-12. In some aspects, the vector constructs illustrated in any of FIGS. 1-12 can modified to replace the “Covid-19 Spike Gene” (a first nucleotide sequence encoding a SARS-CoV-2 protein) and the “Covid-19 Gene-2” (a second nucleotide sequence encoding a SARS-CoV-2 protein) with nucleotide sequences encoding any combinations of pathogen antigen or antigenic fragment thereof disclosed herein.

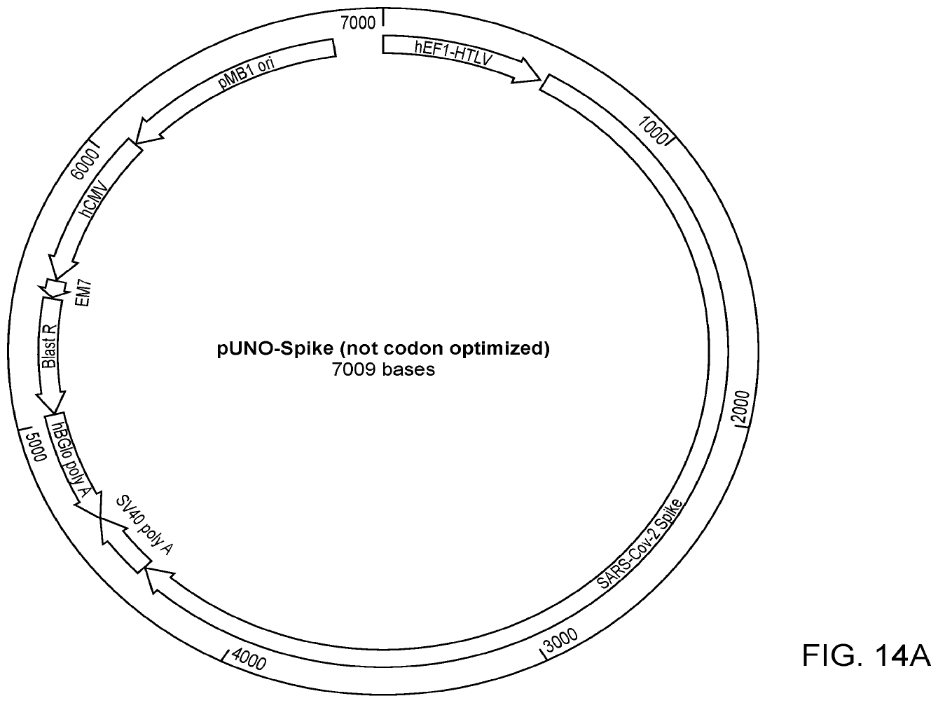

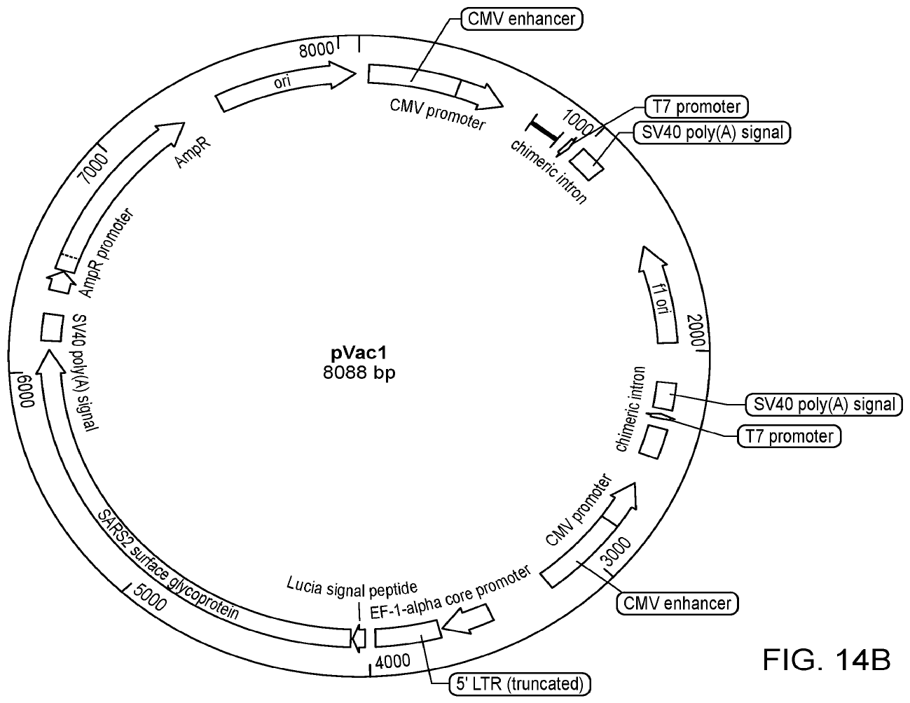

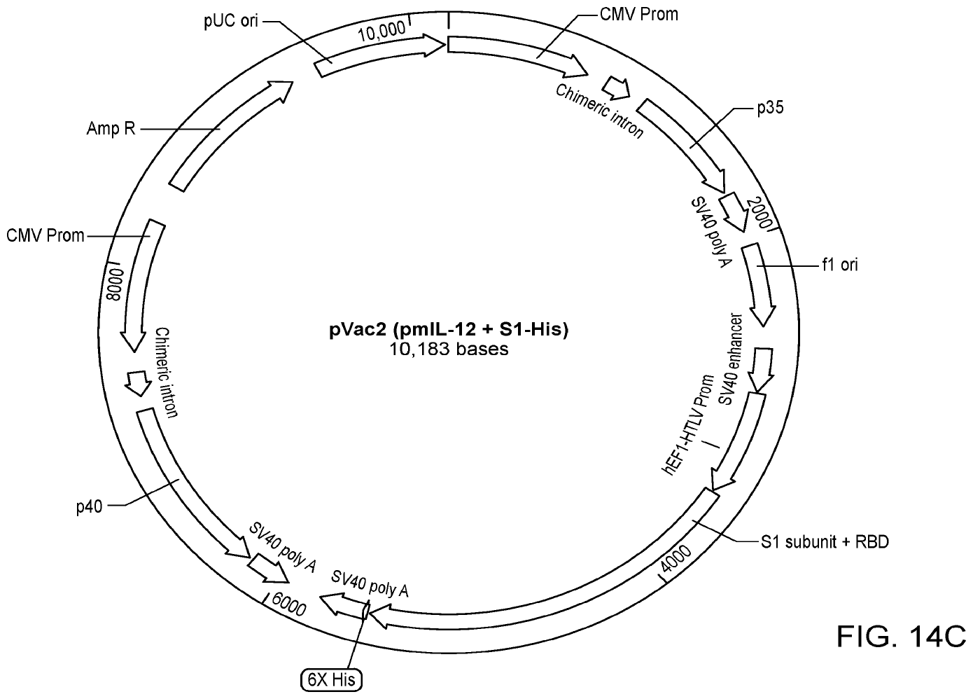

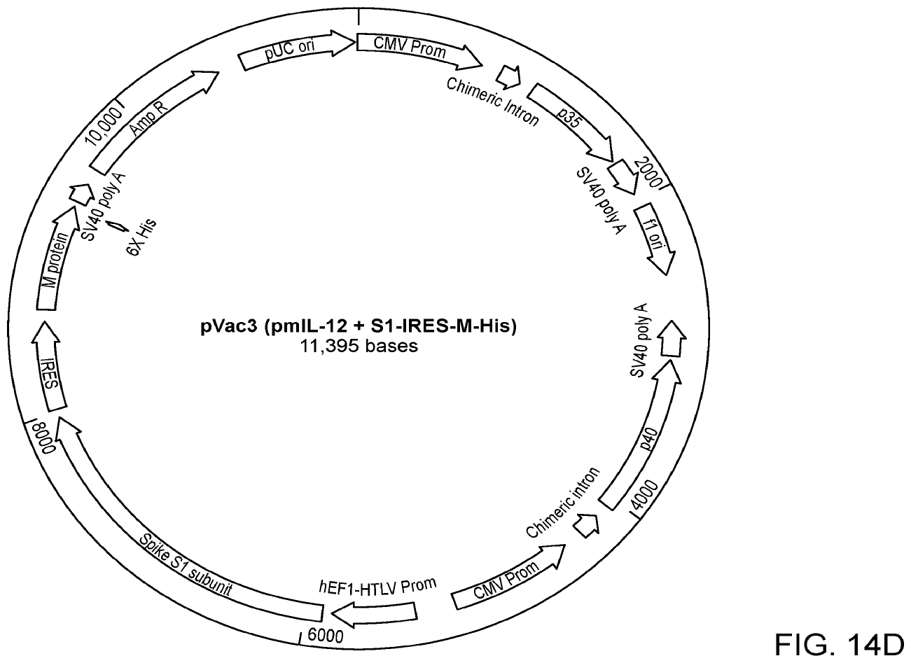

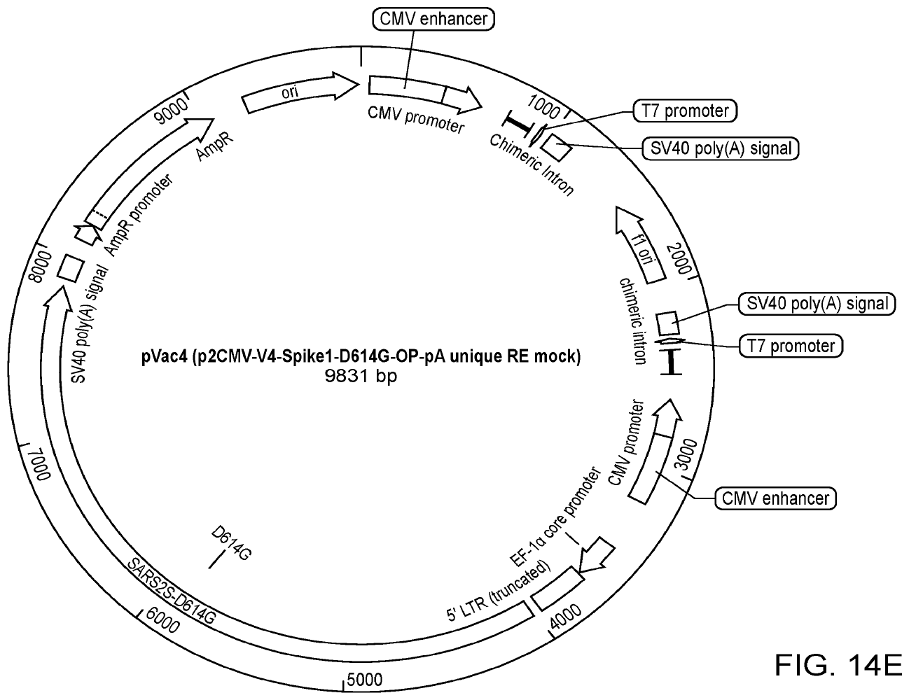

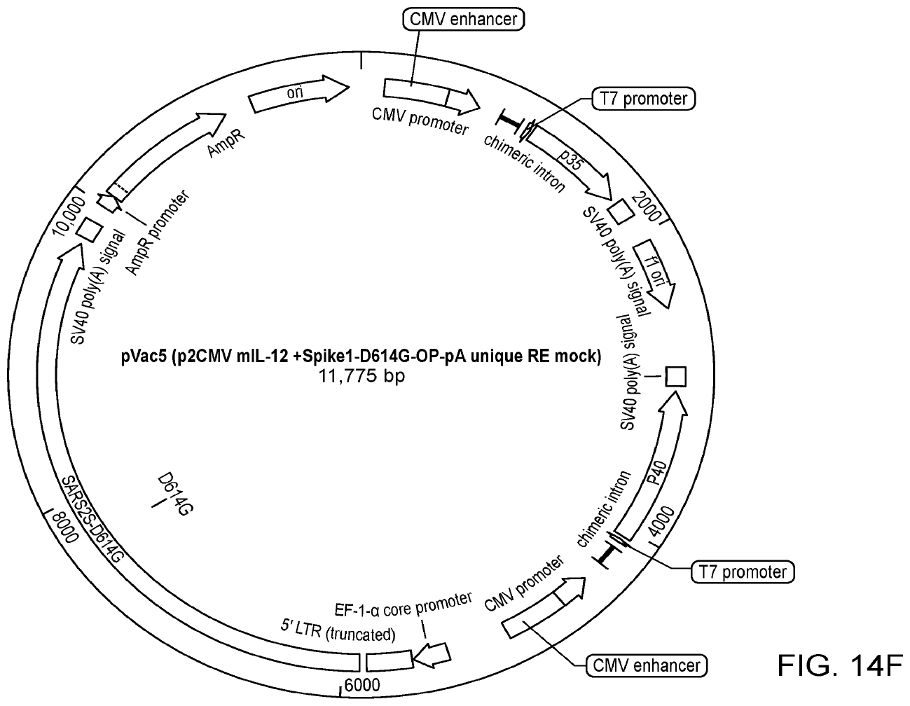

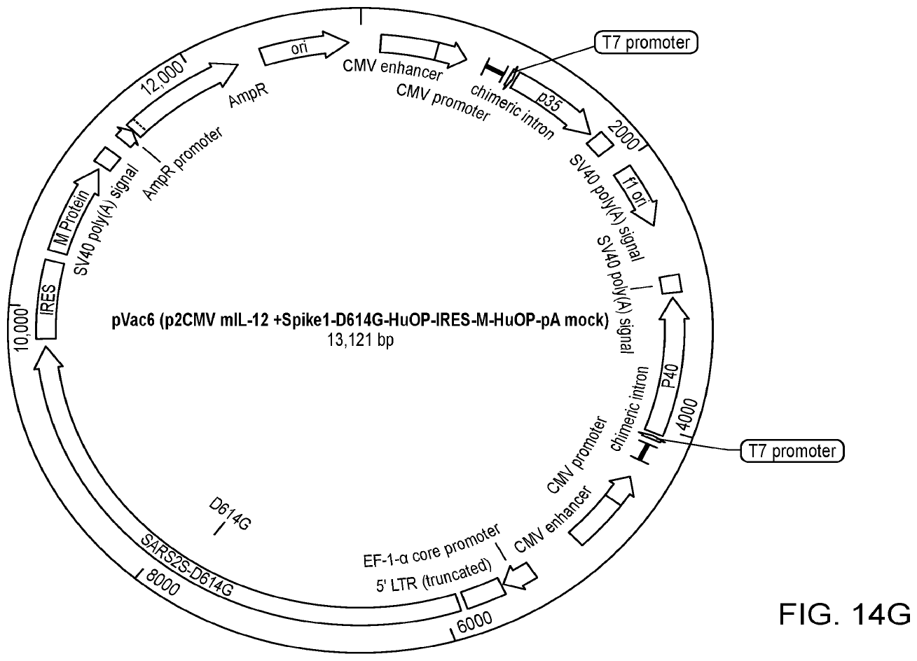

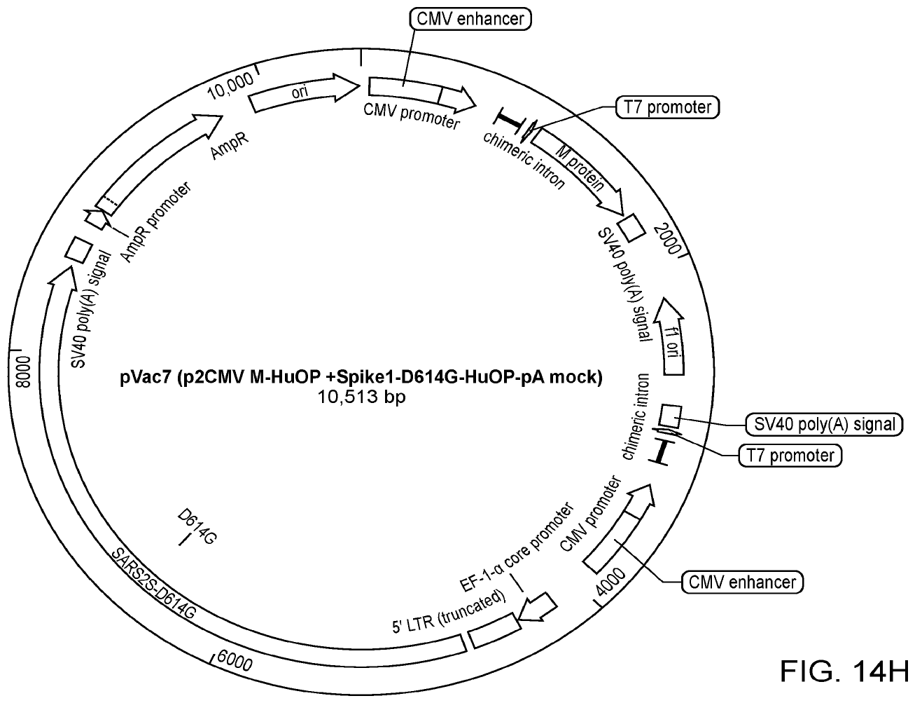

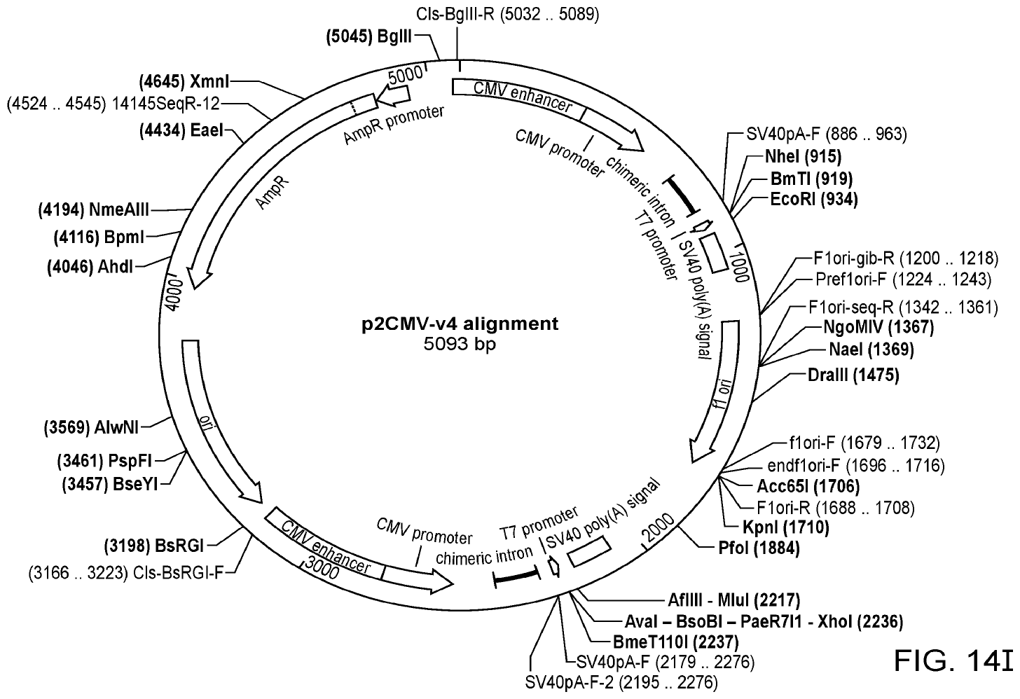

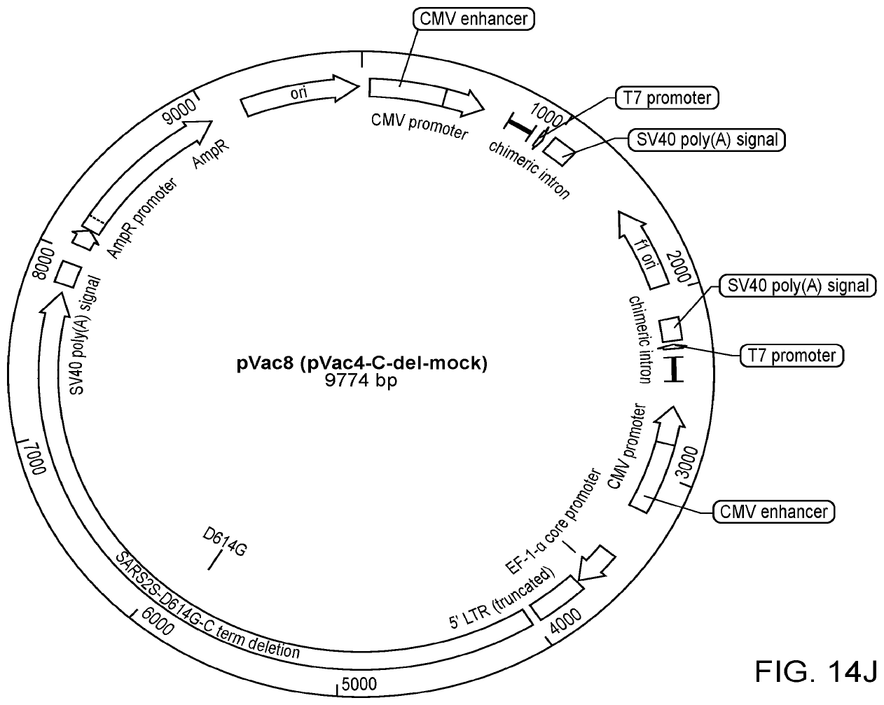

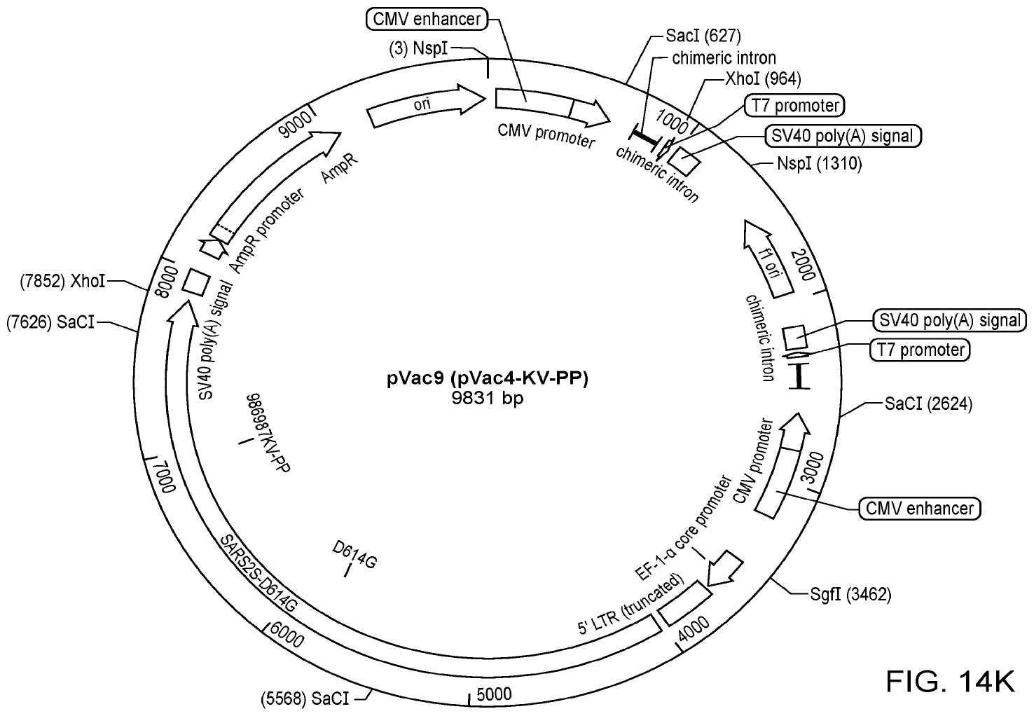

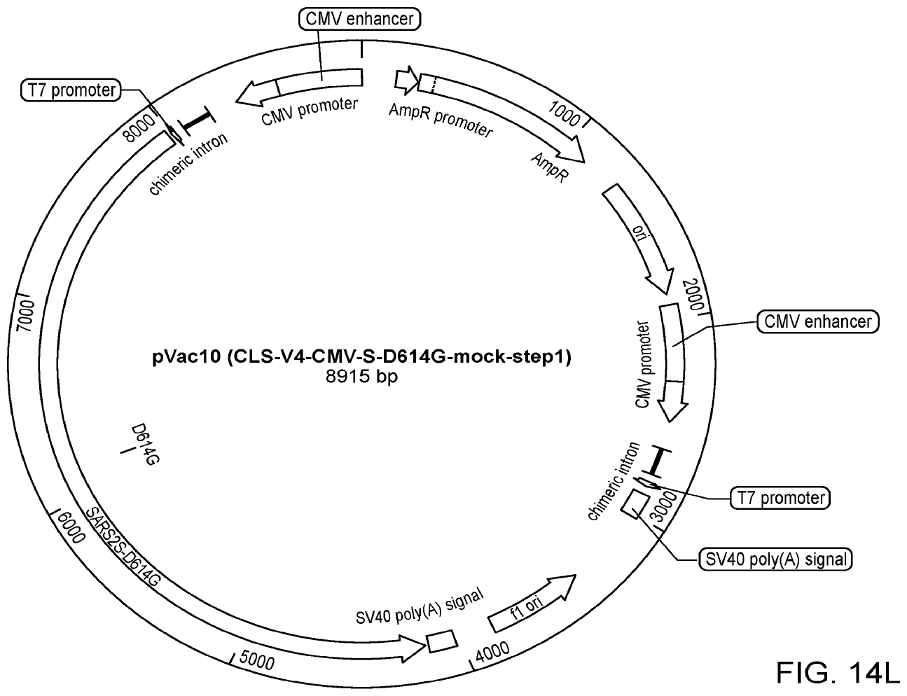

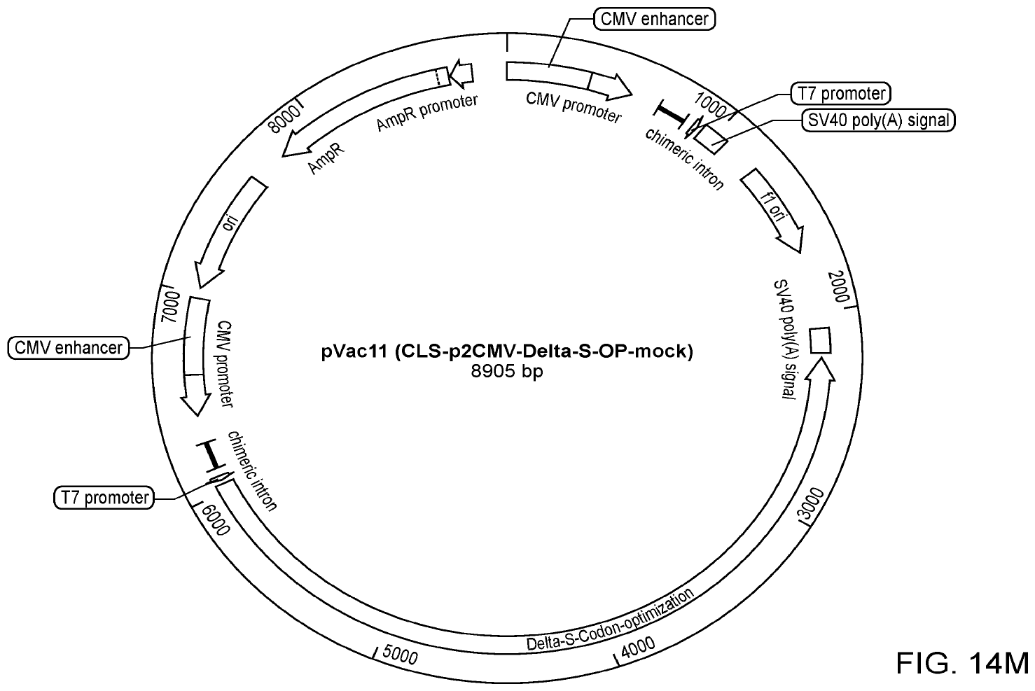

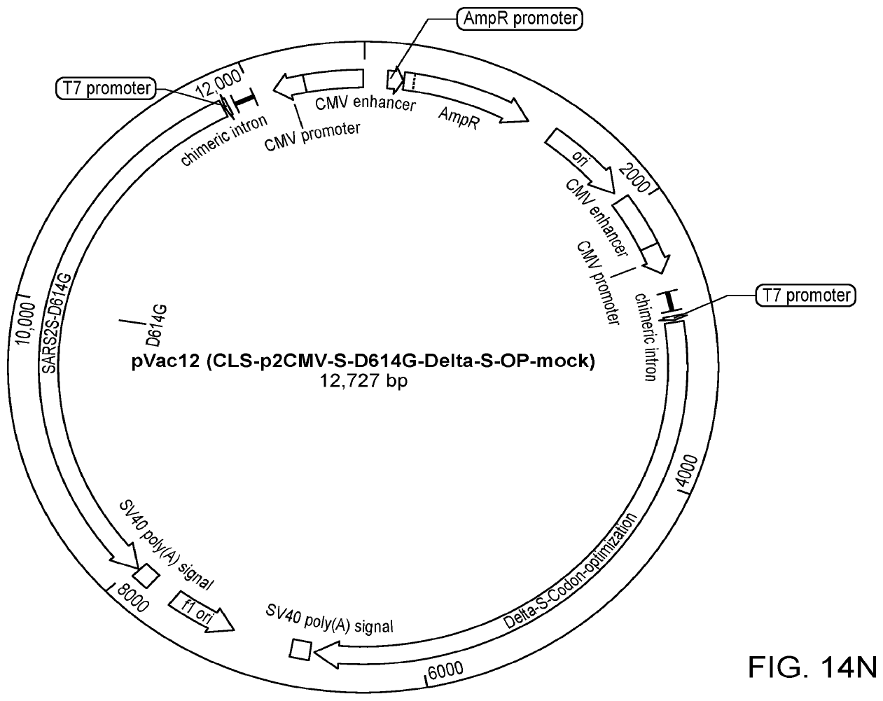

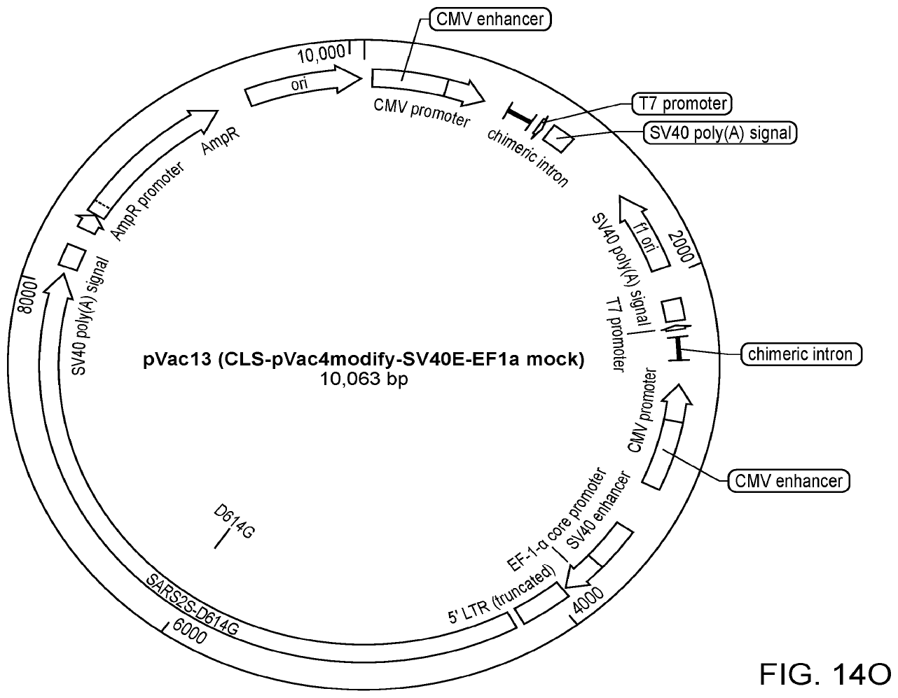

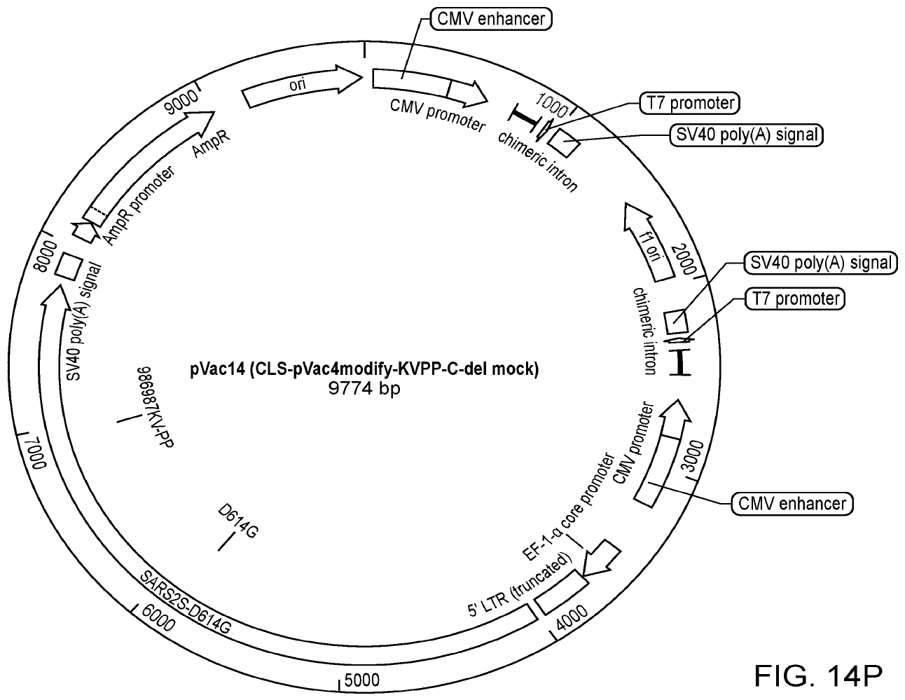

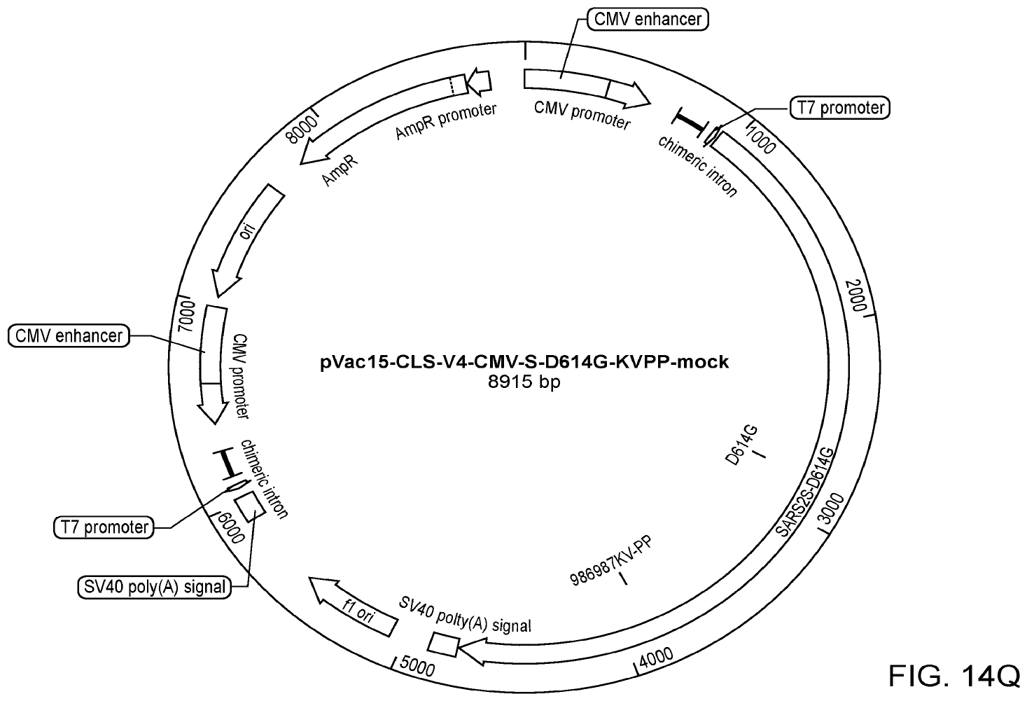

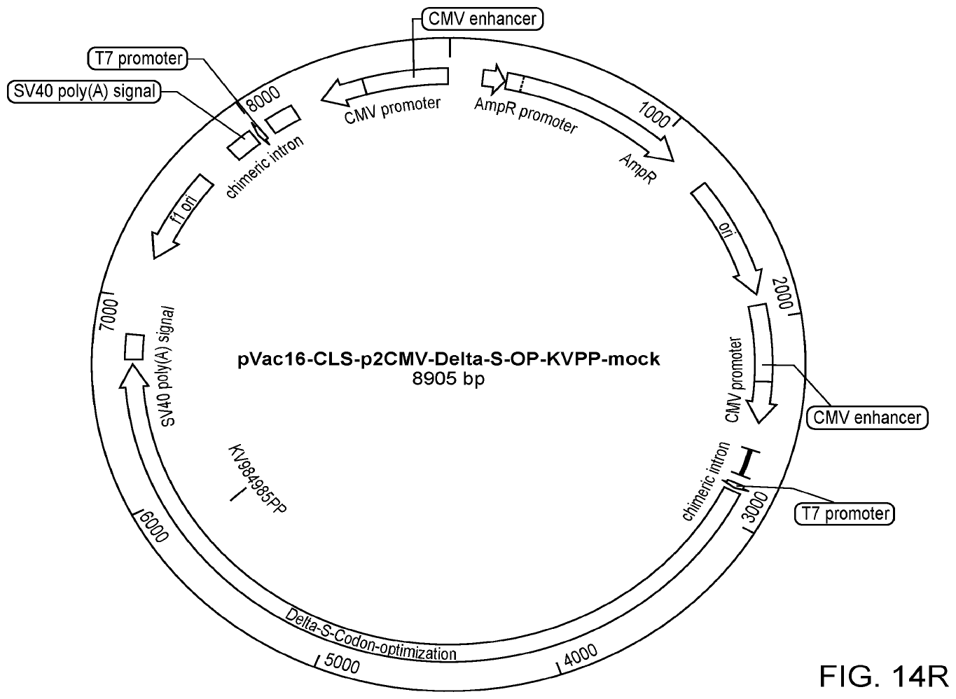

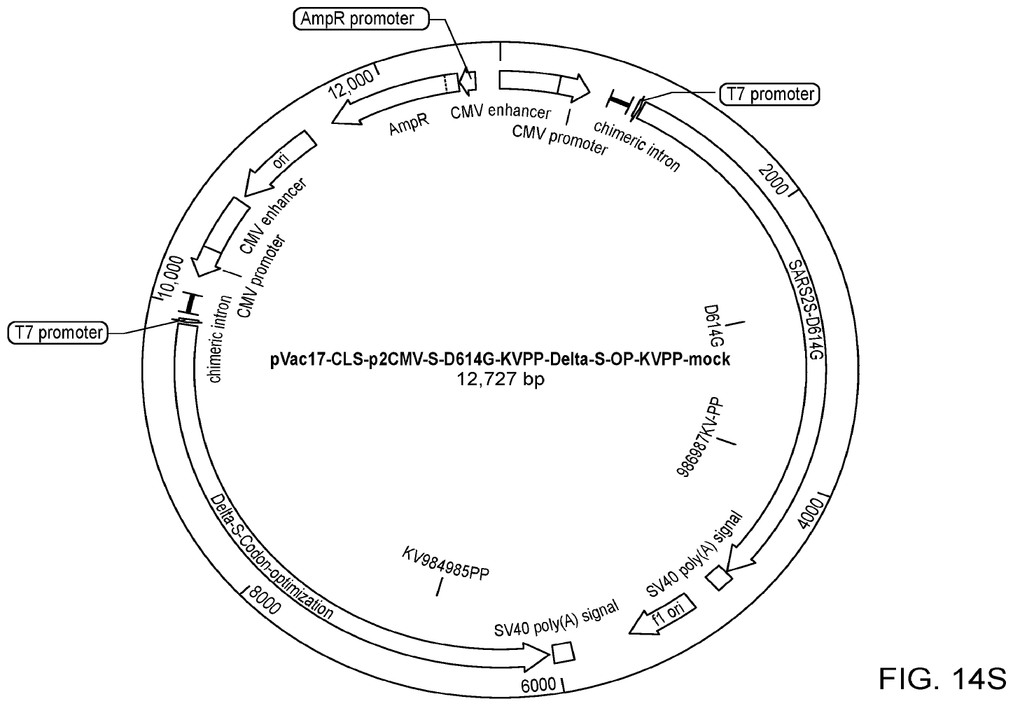

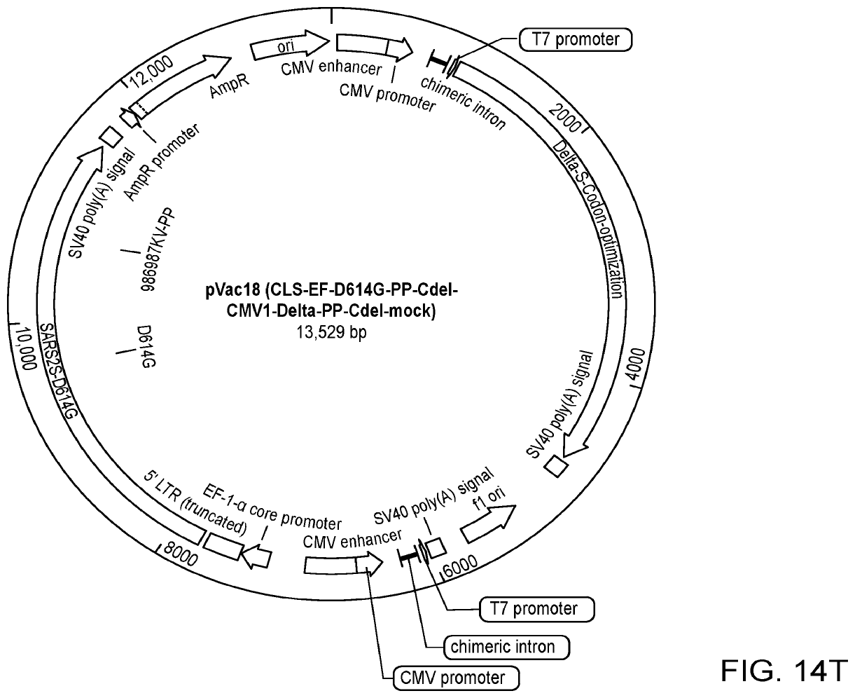

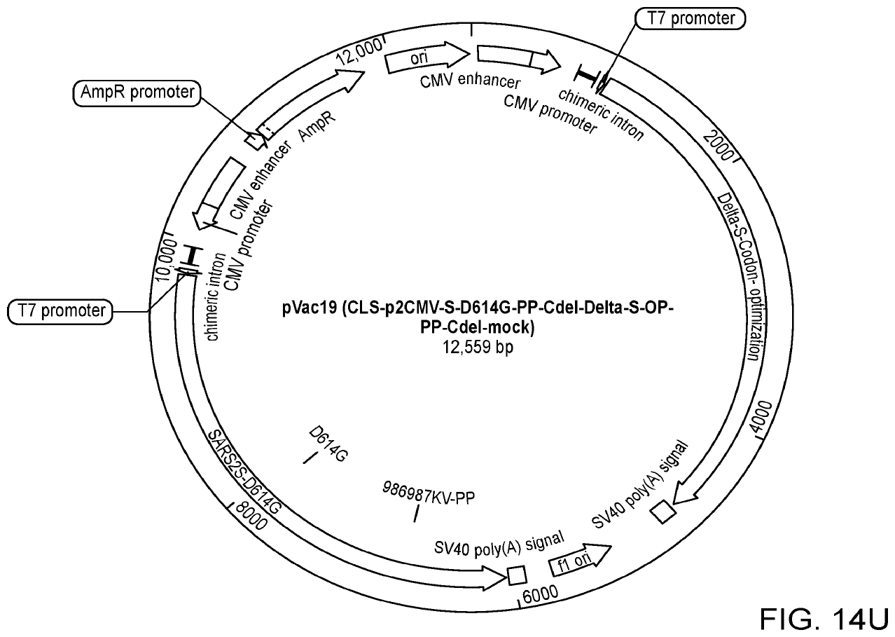

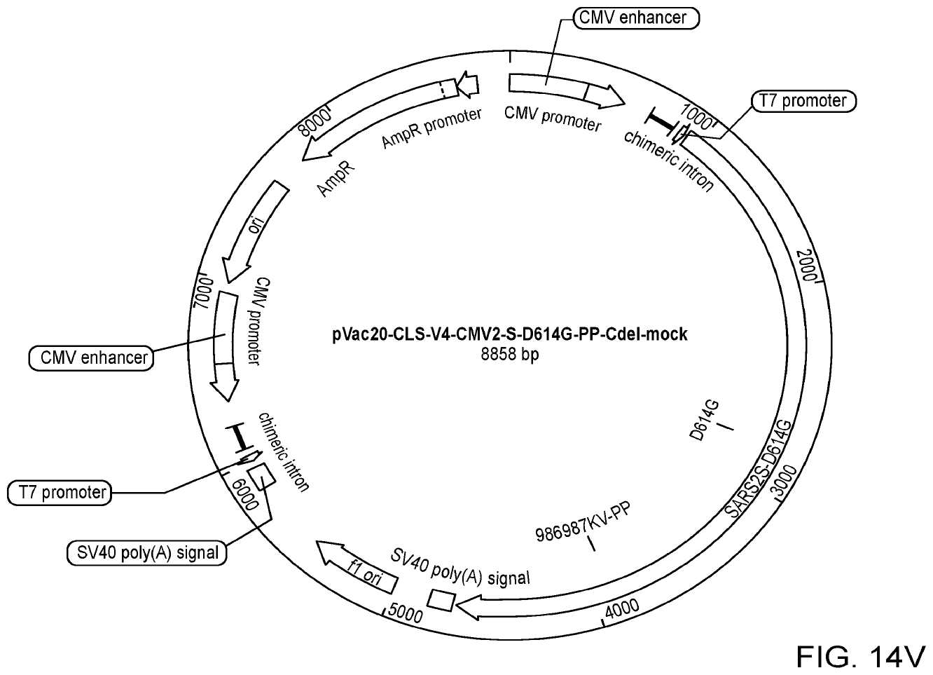

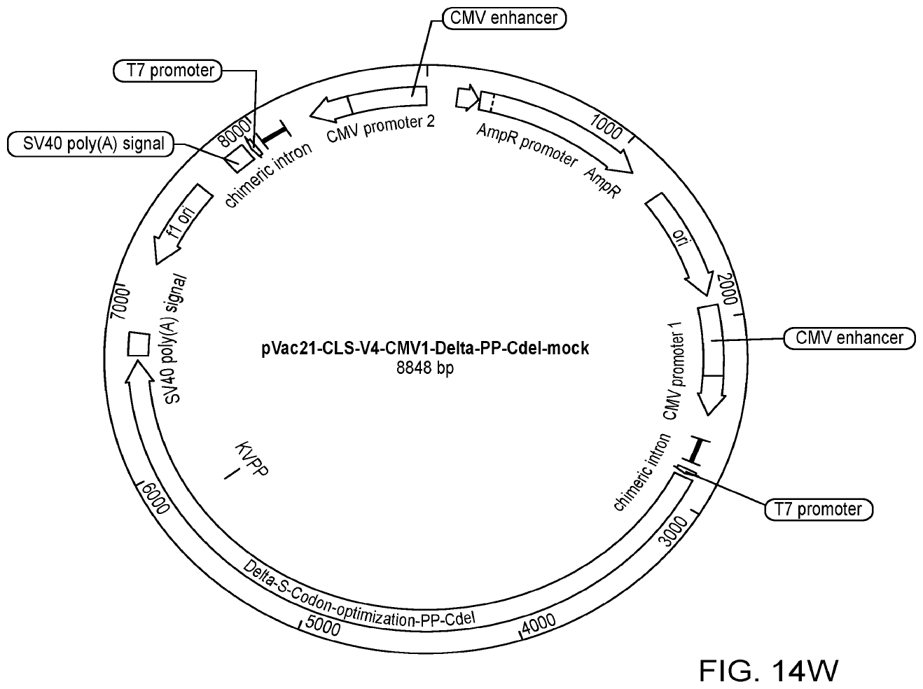

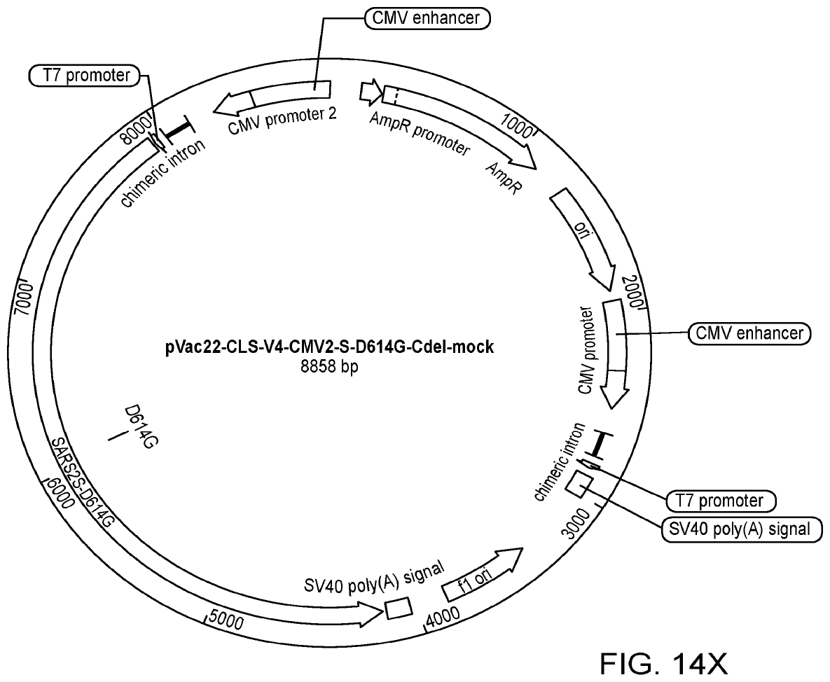

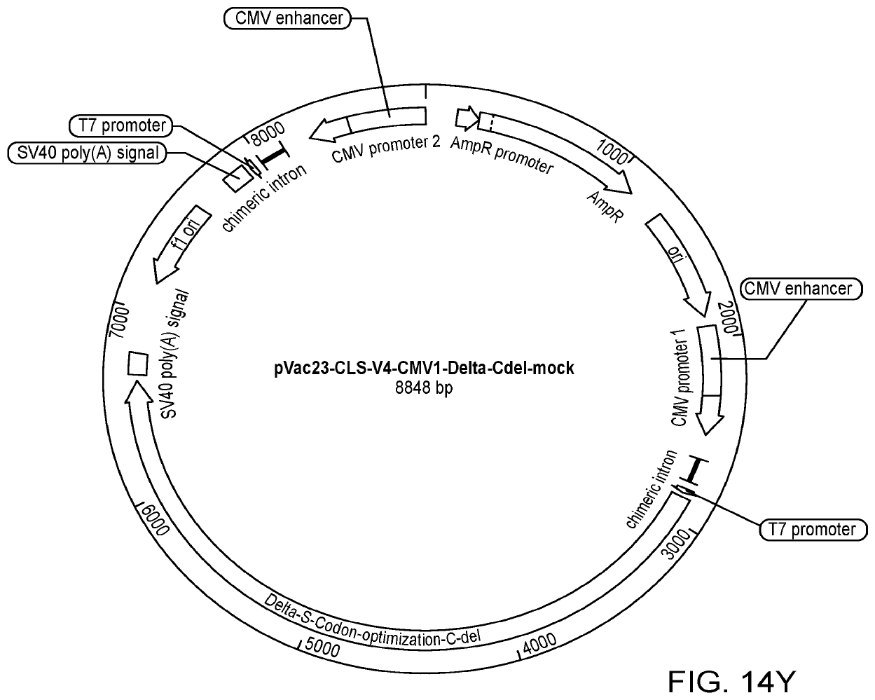

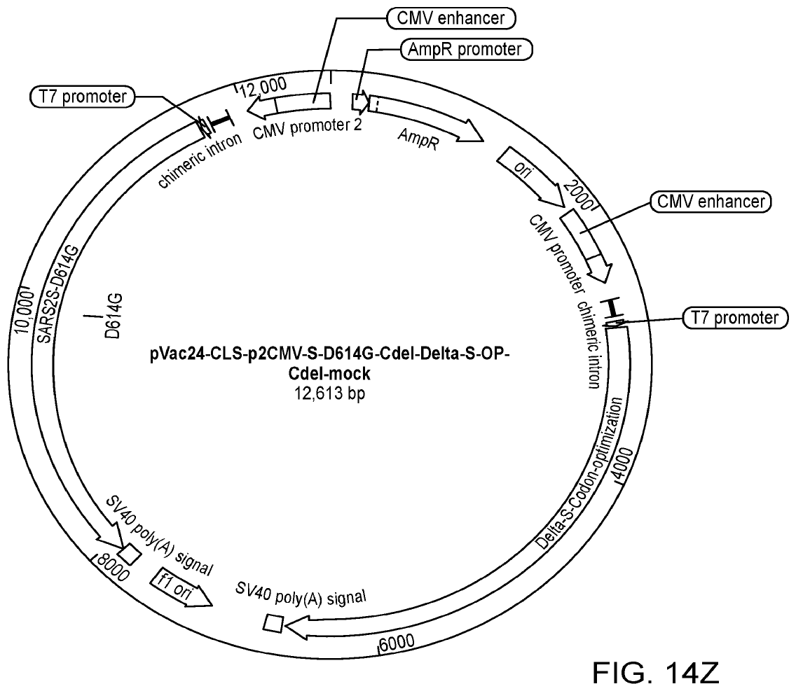

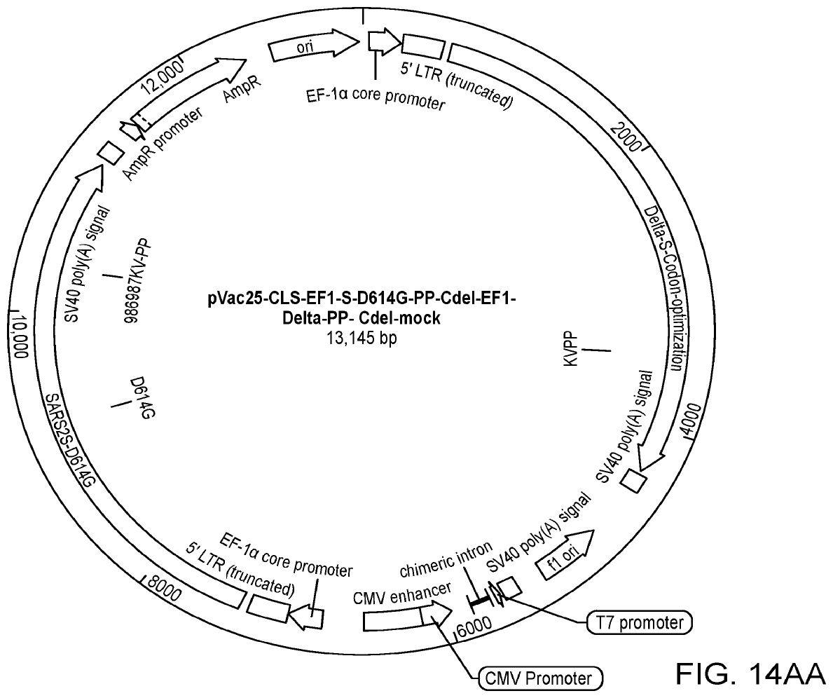

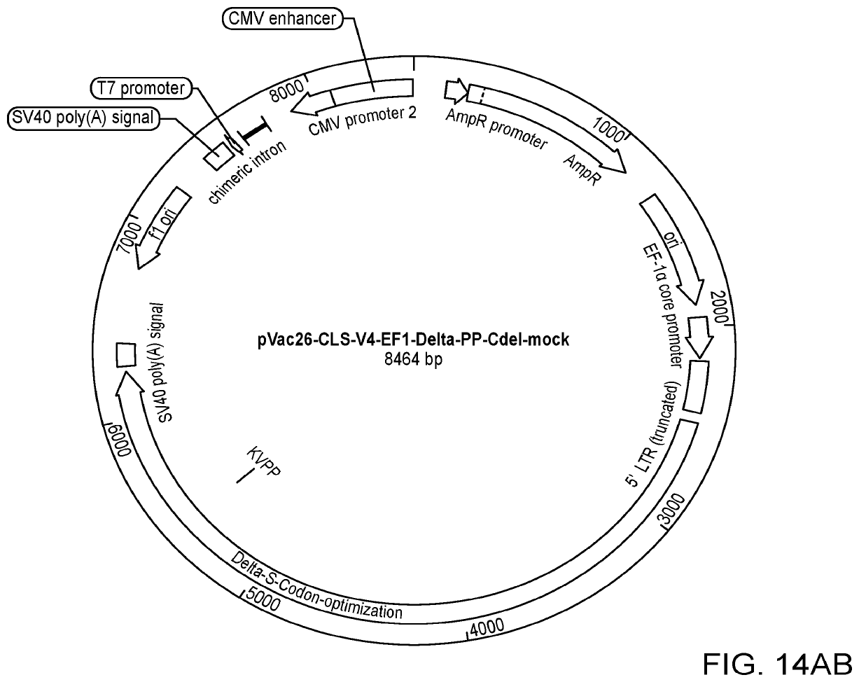

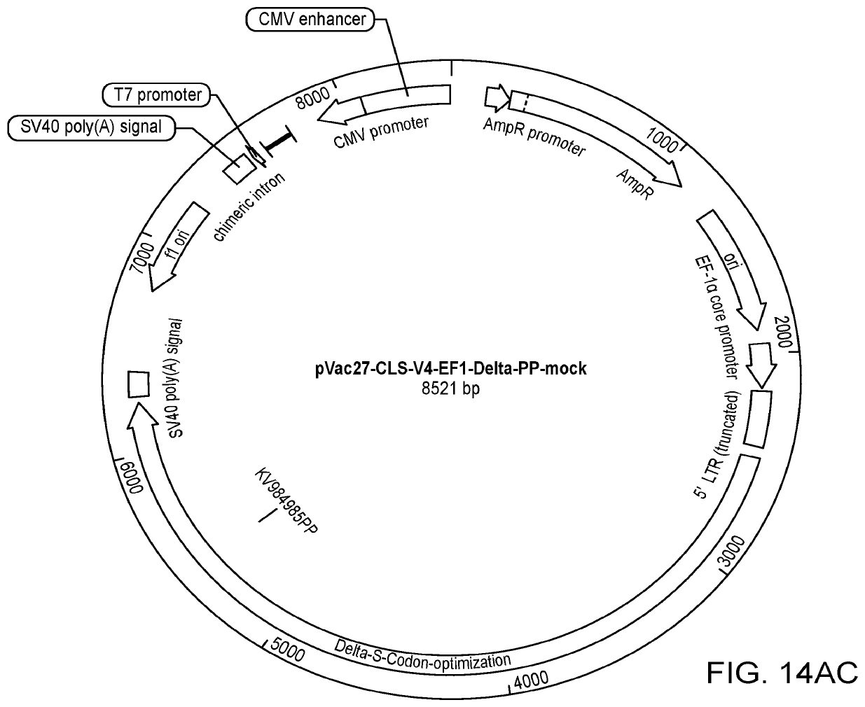

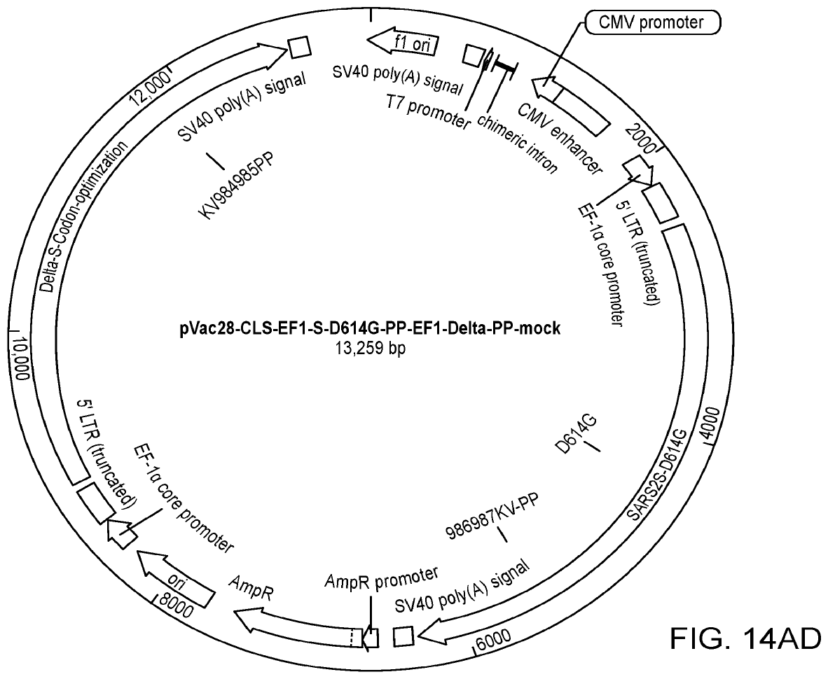

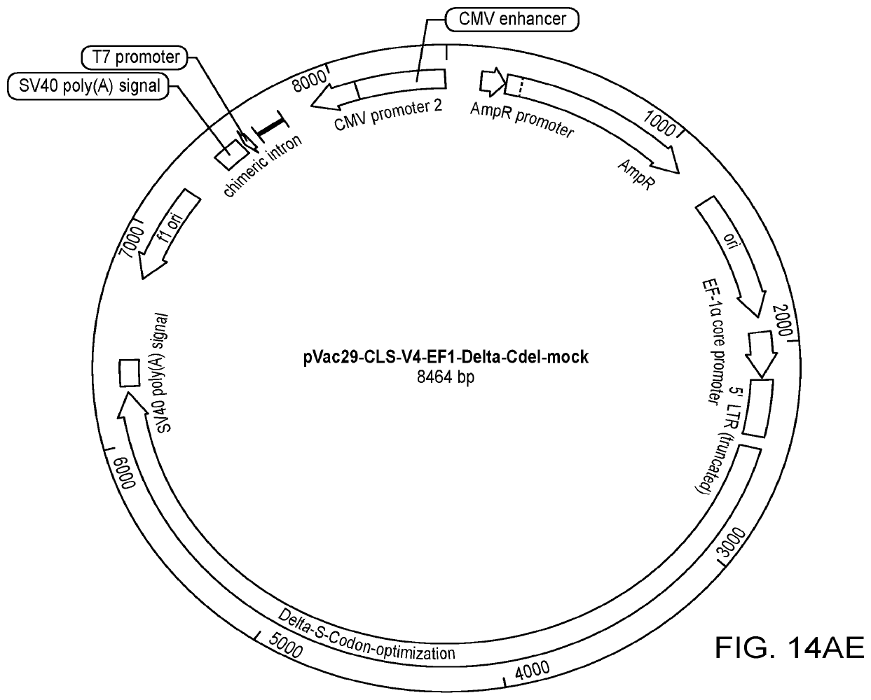

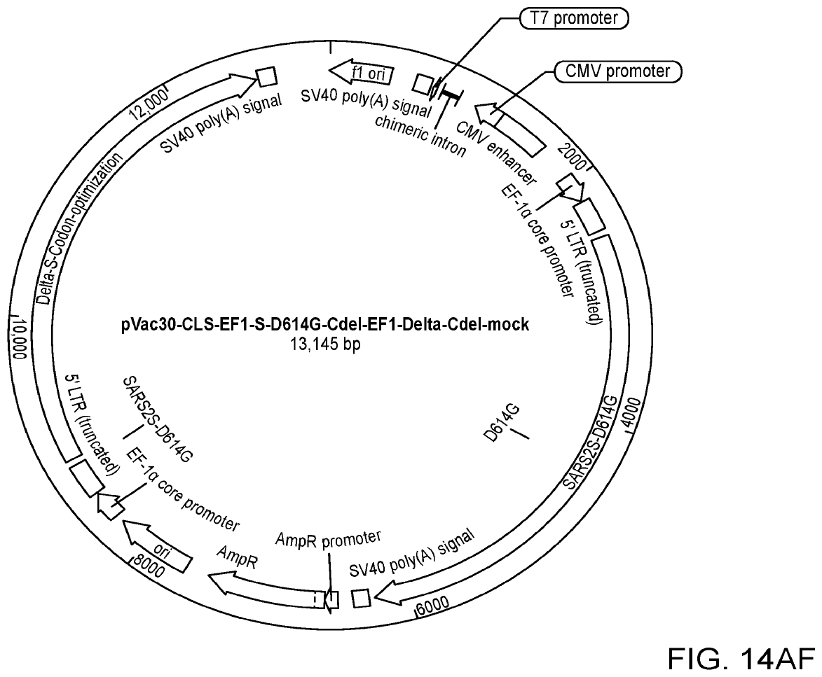

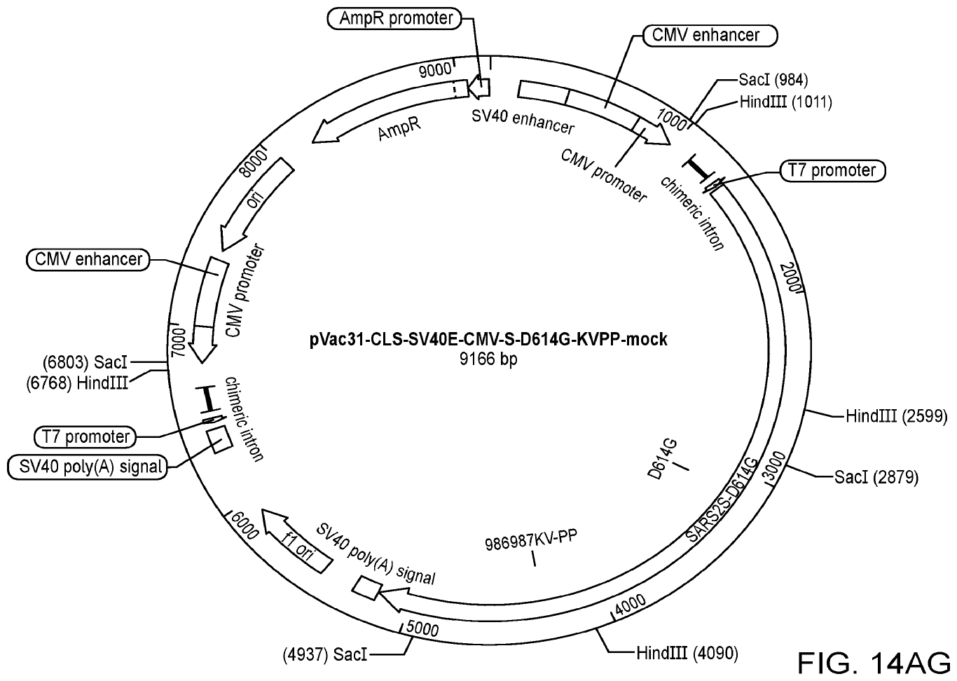

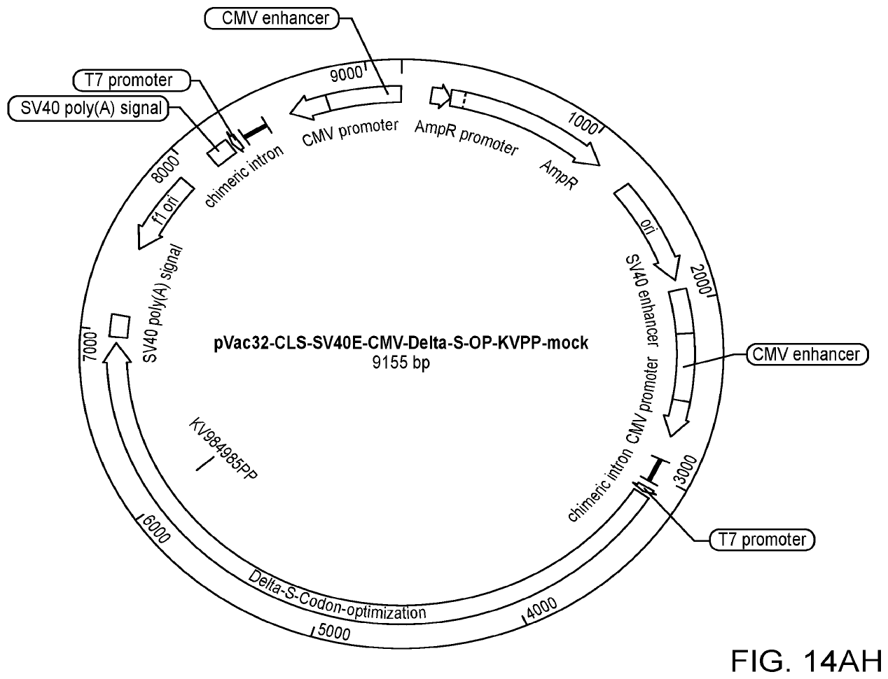

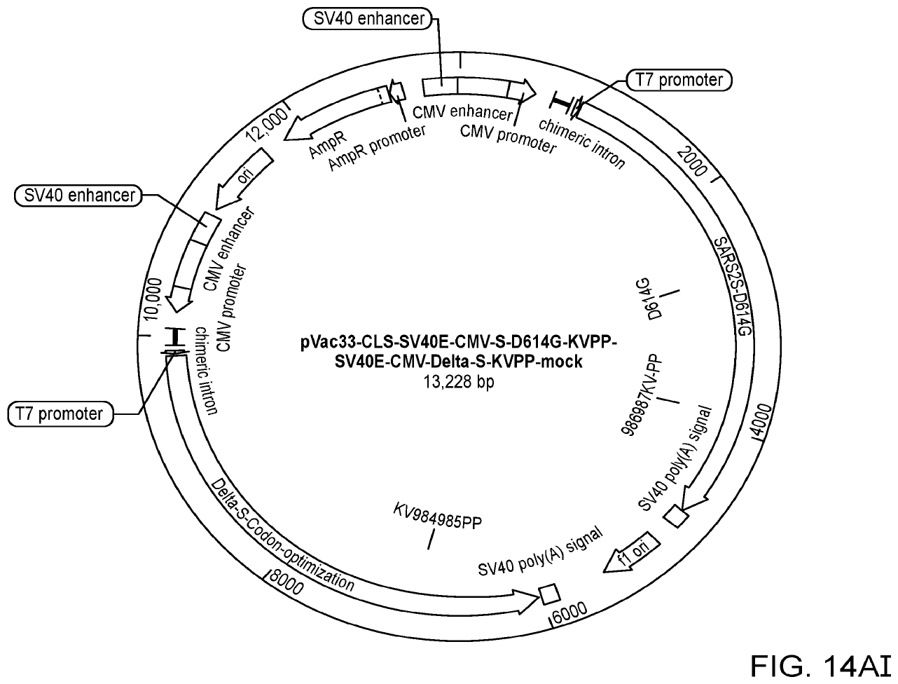

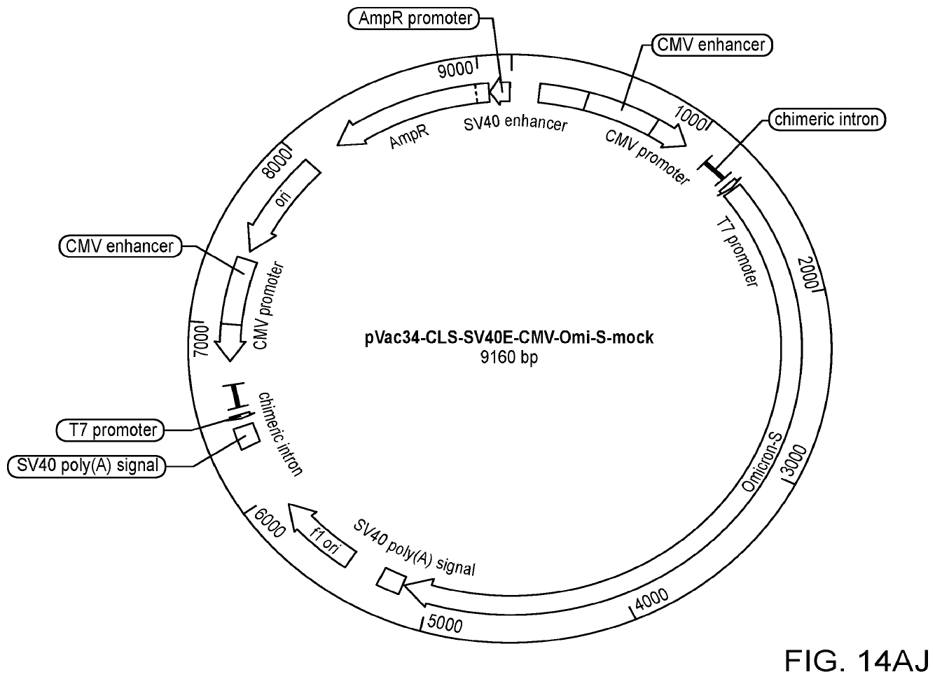

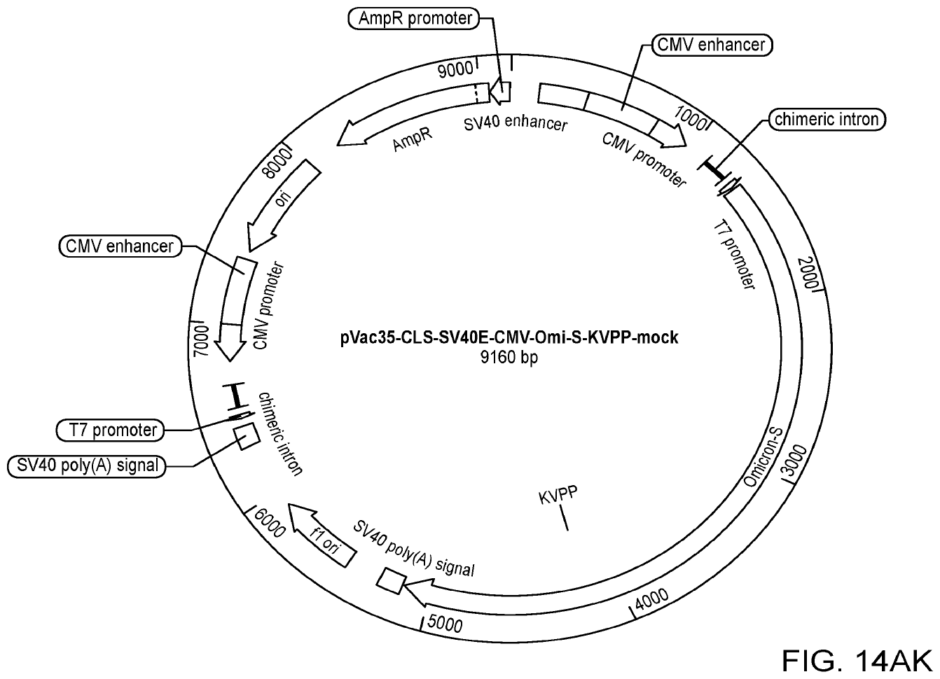

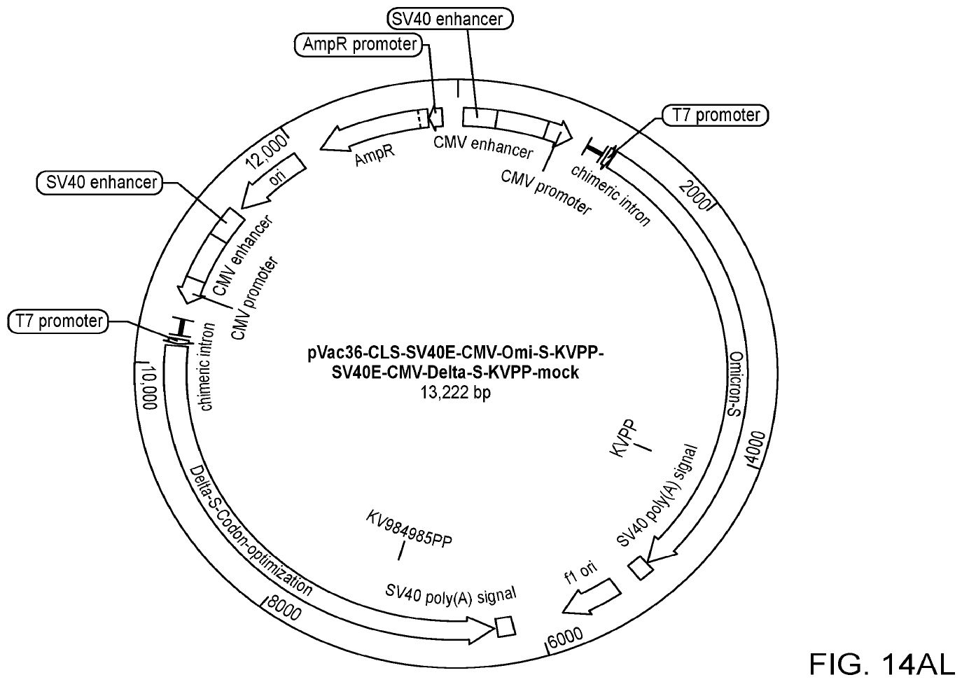

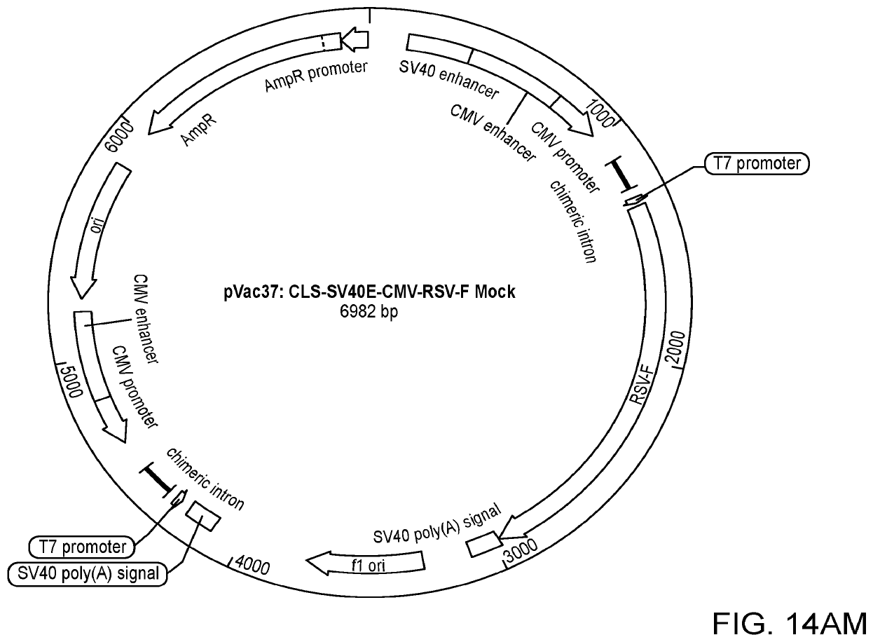

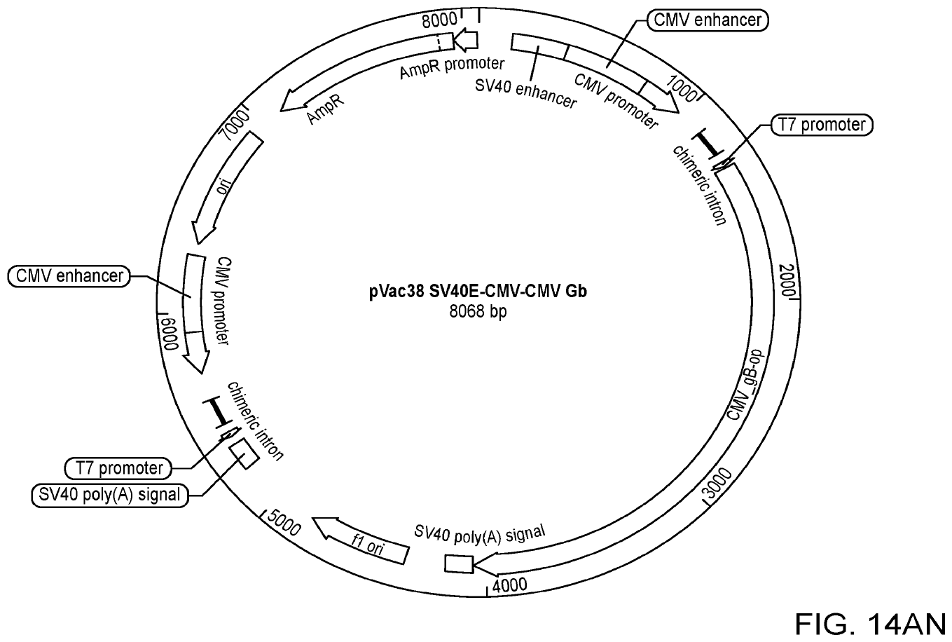

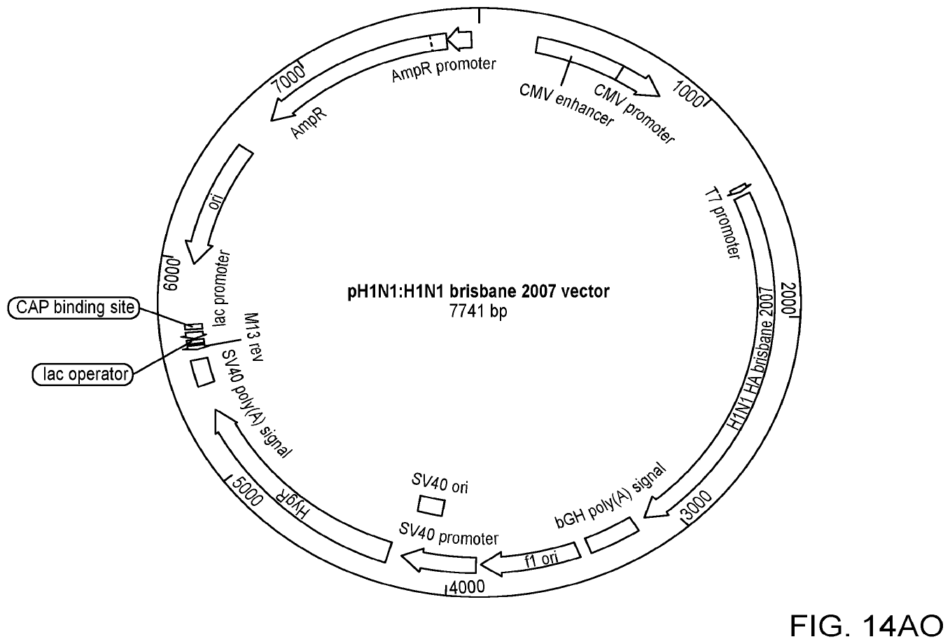

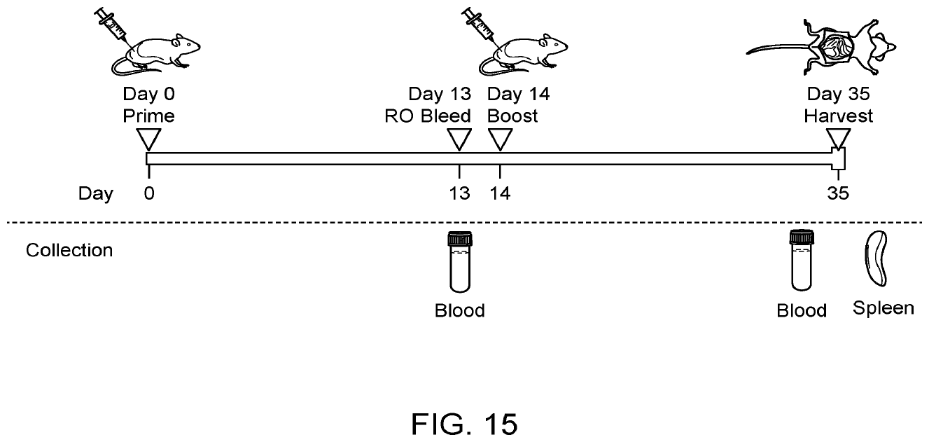

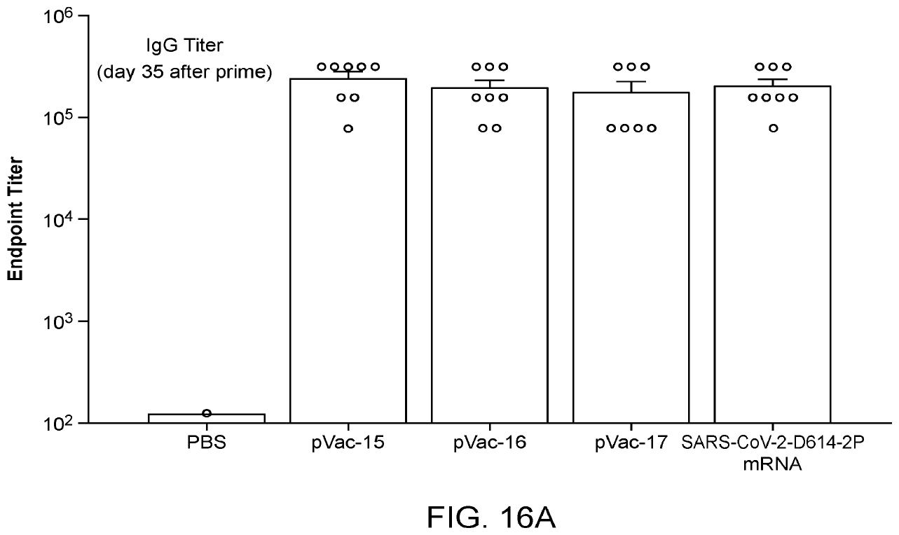

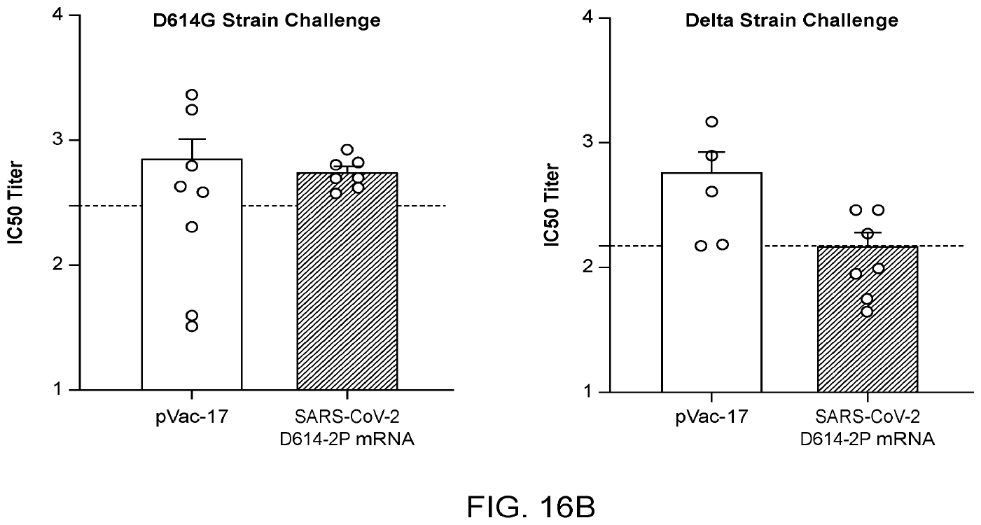

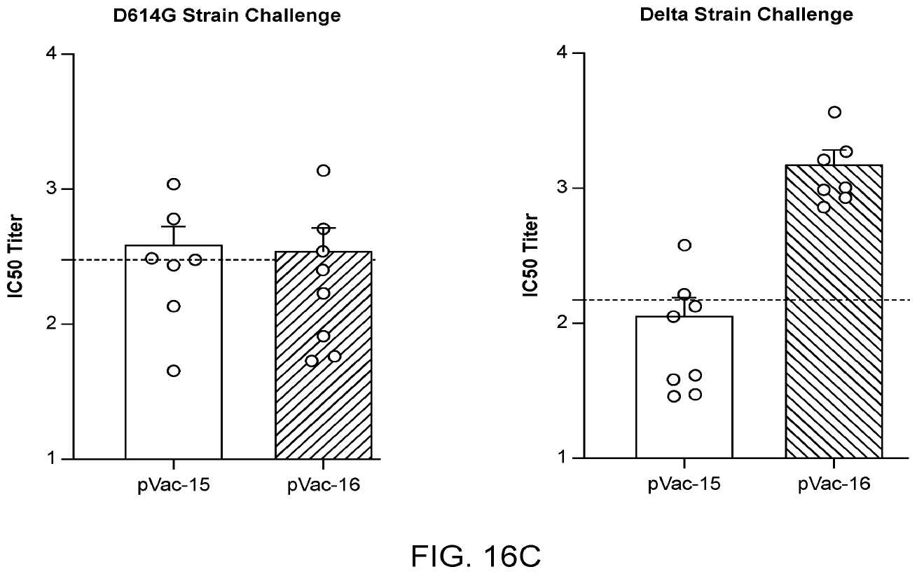

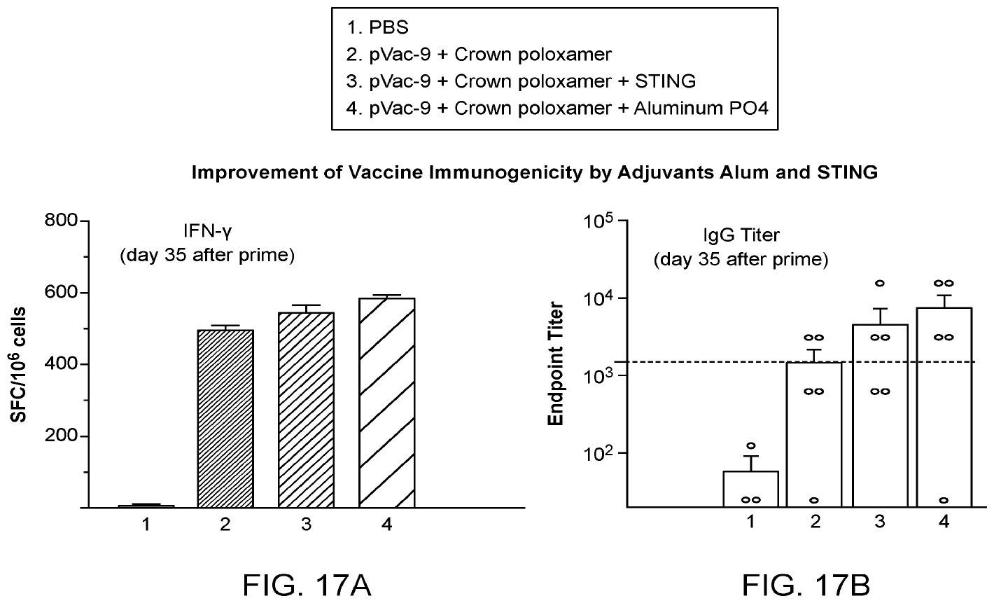

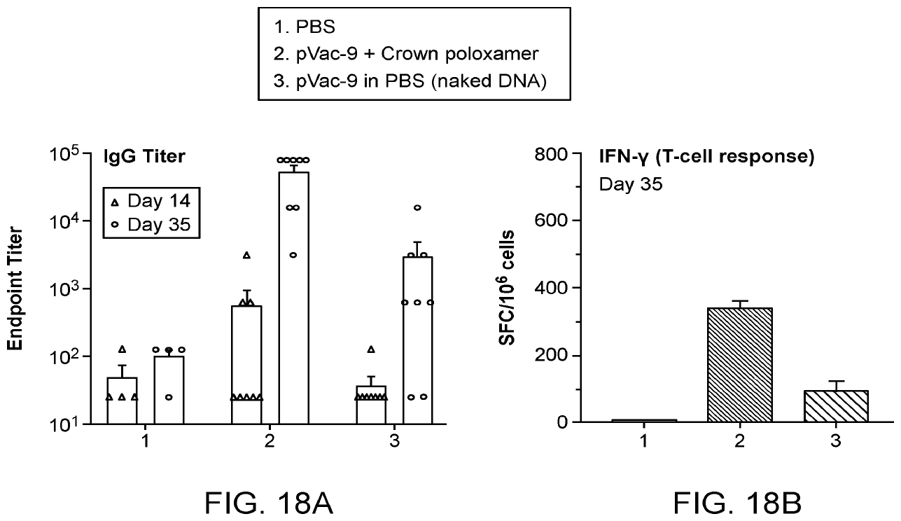

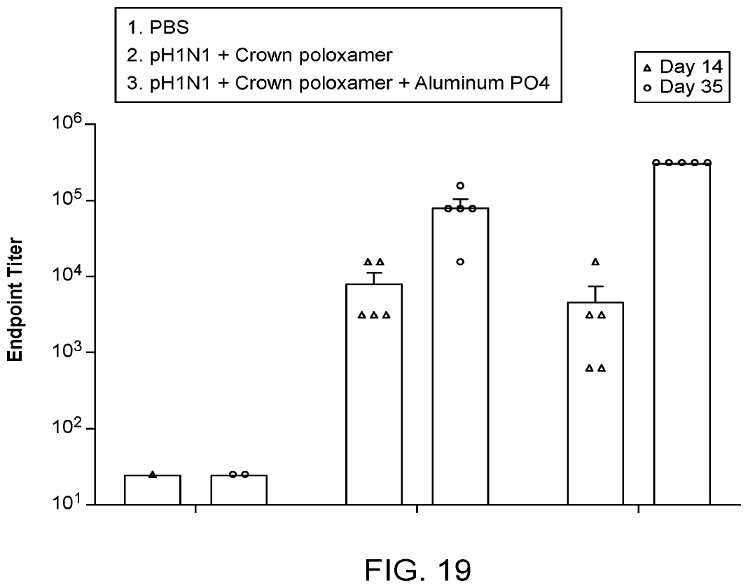

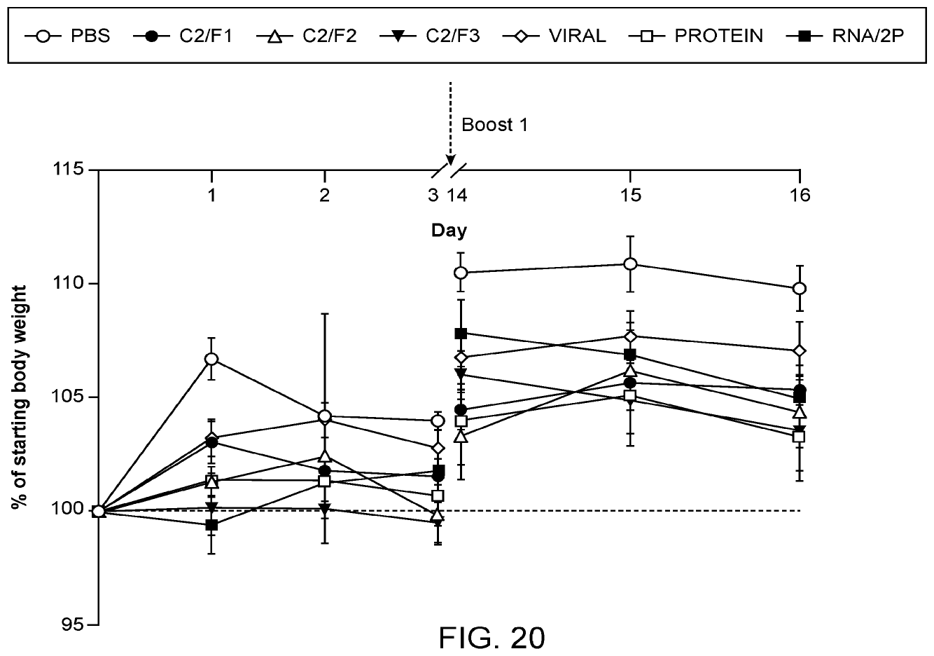

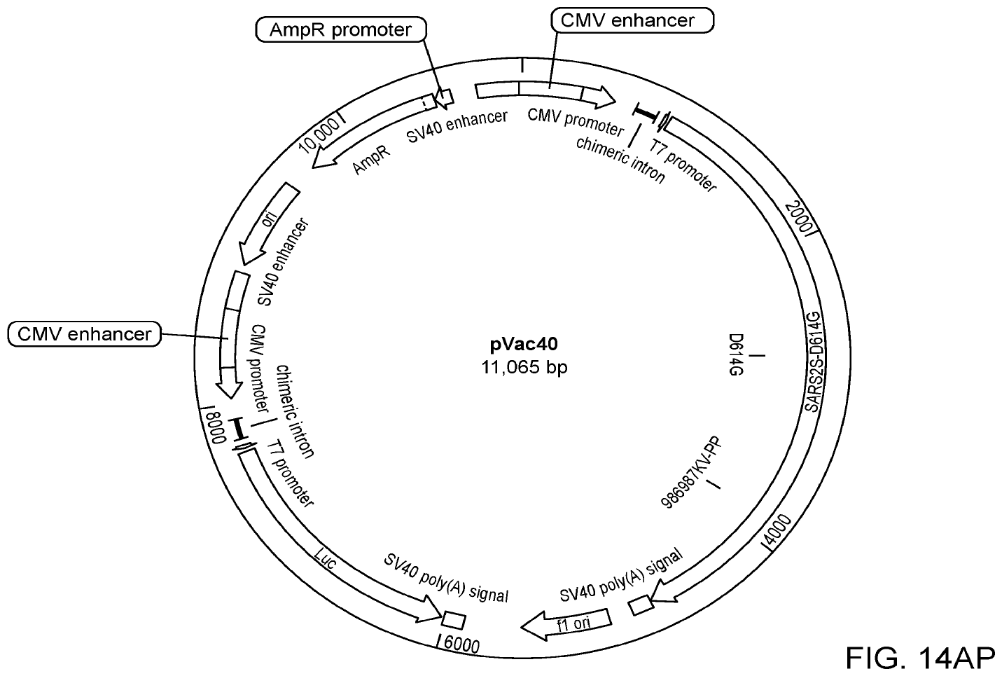

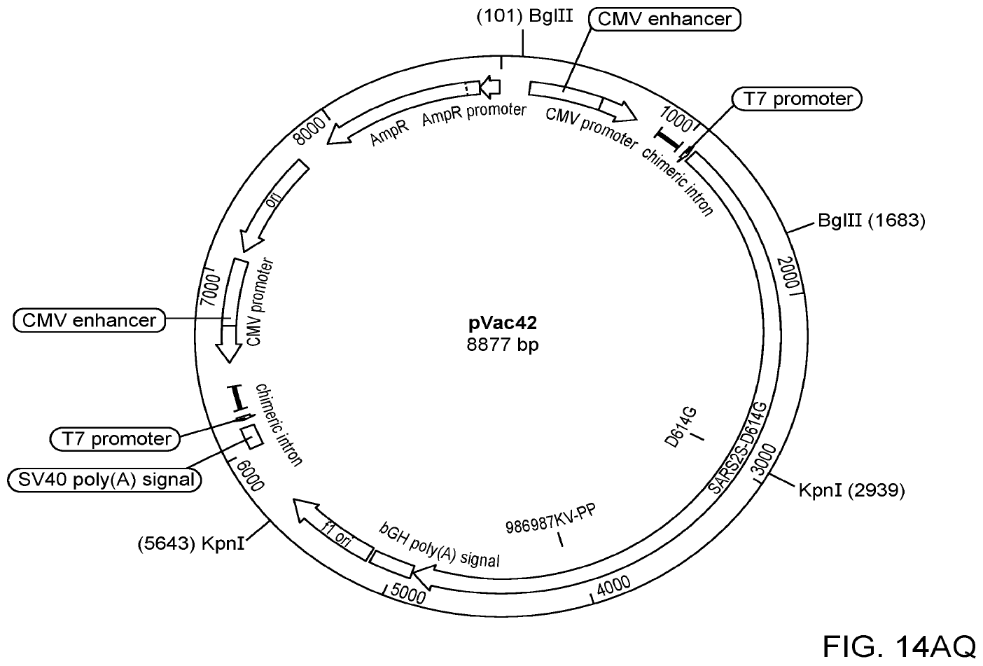

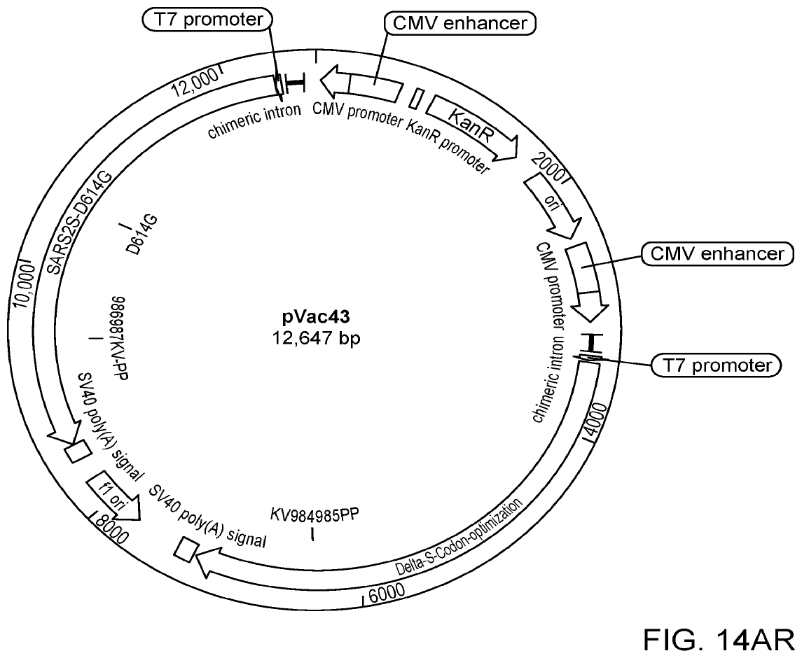

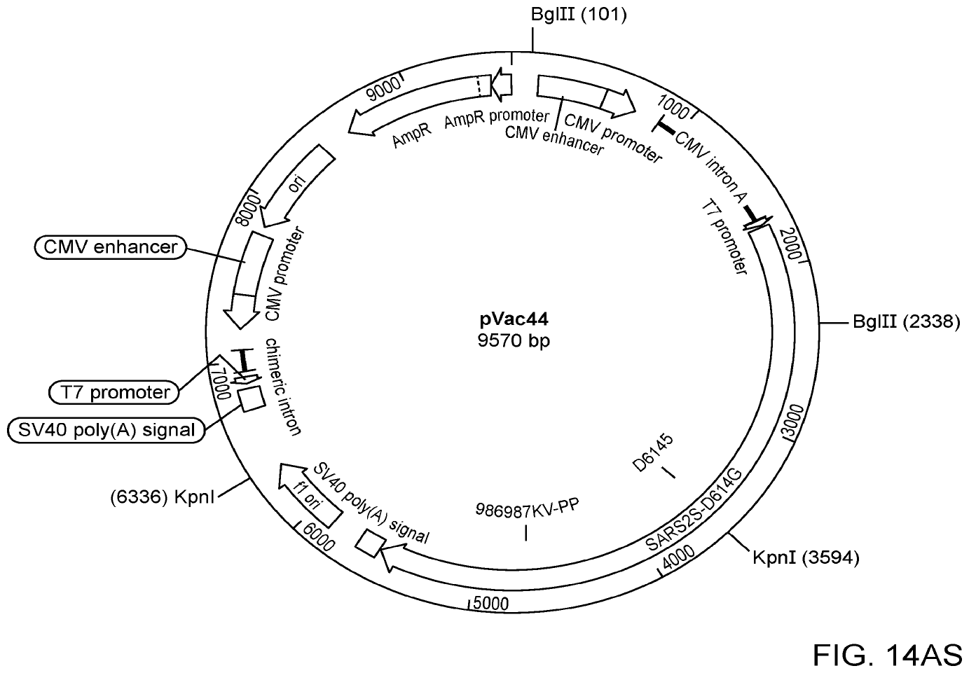

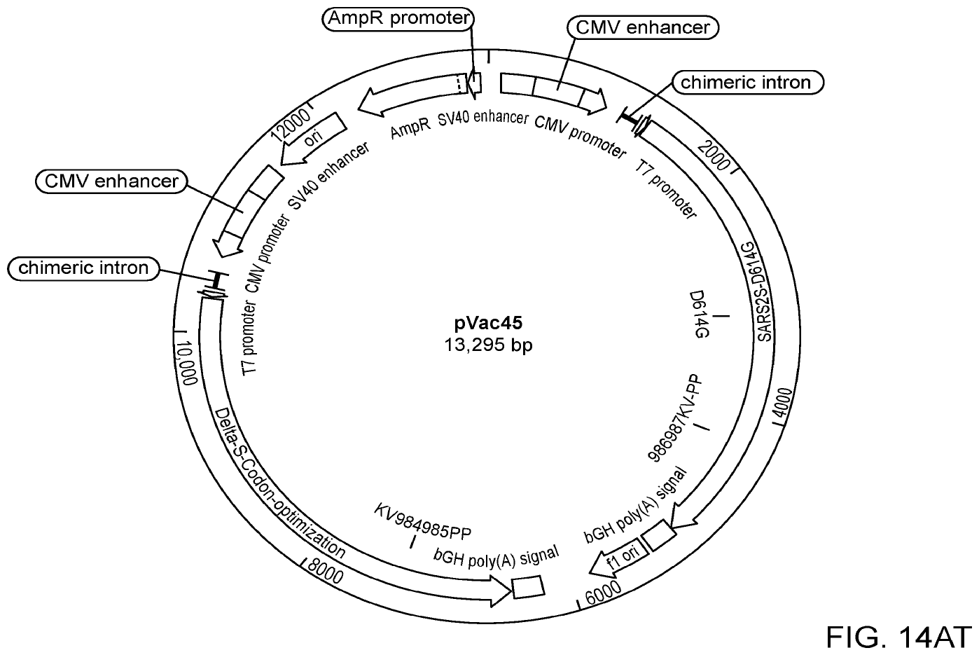

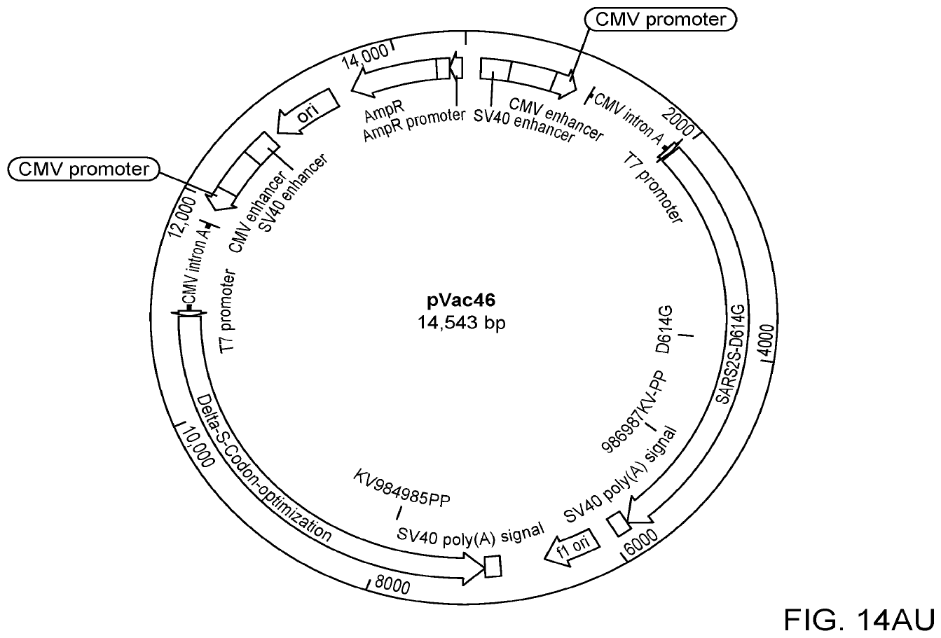

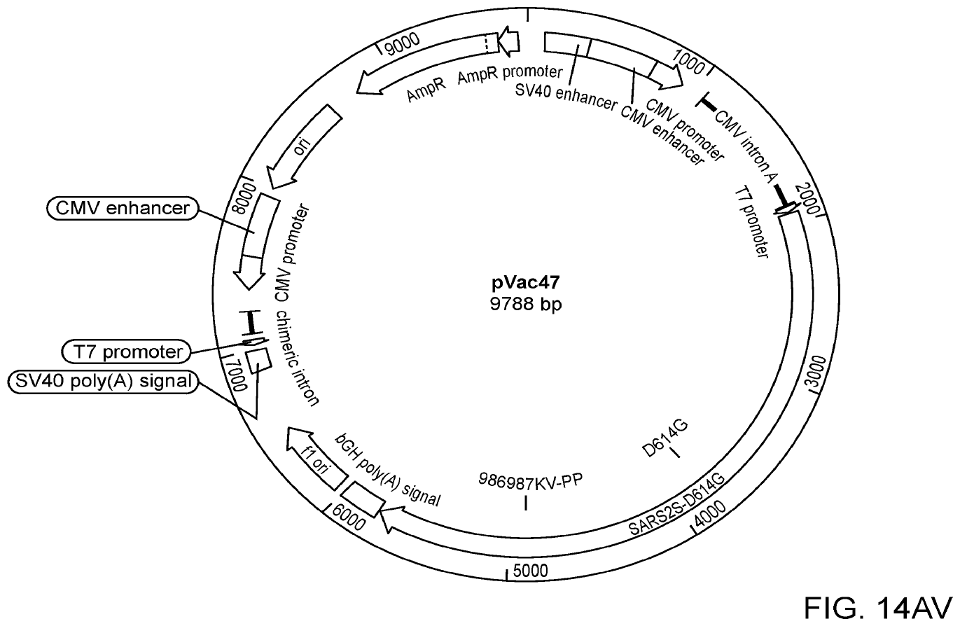

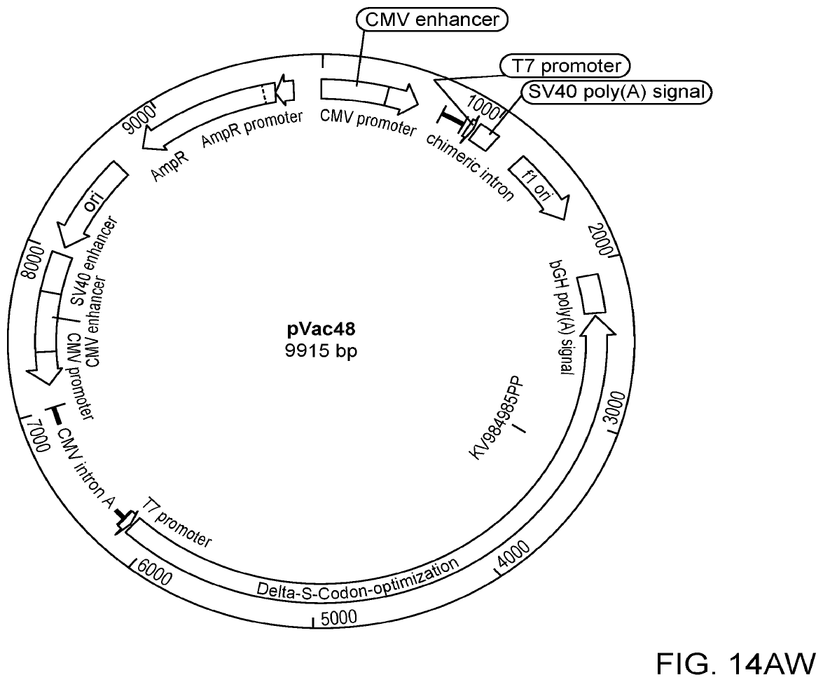

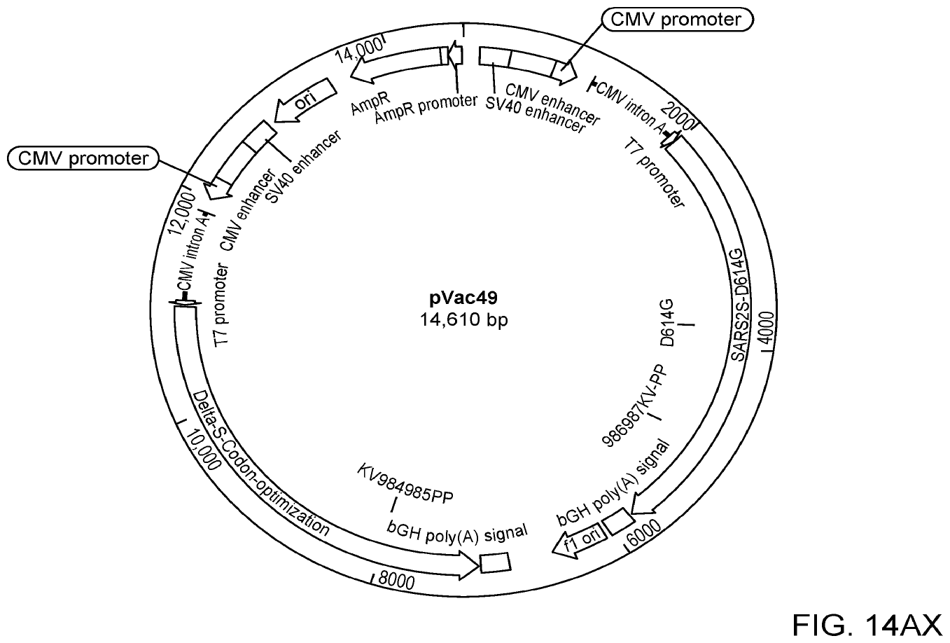

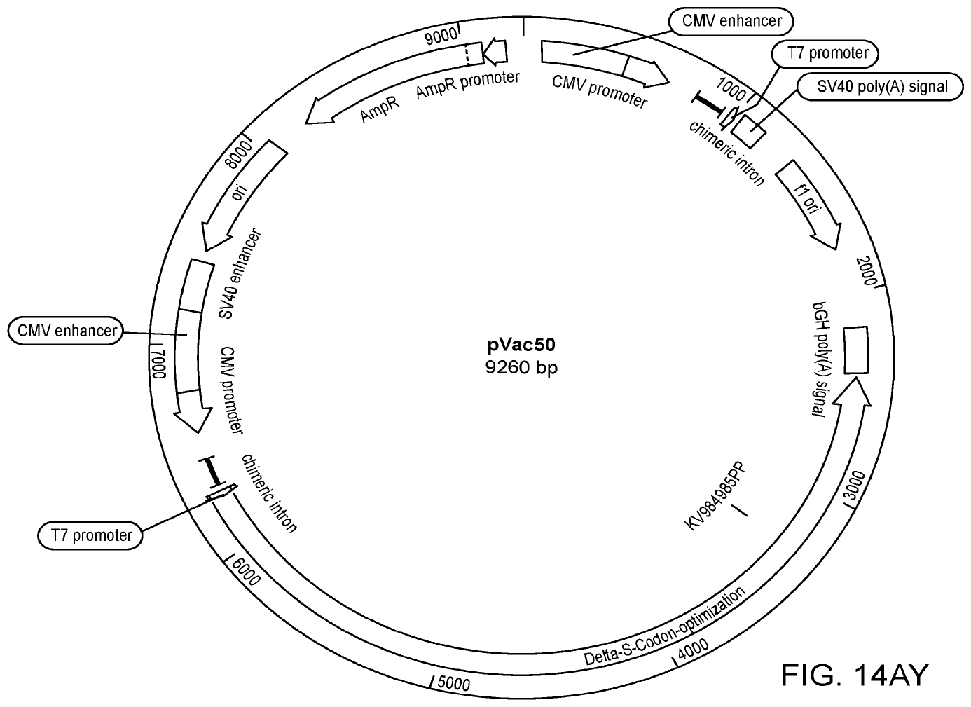

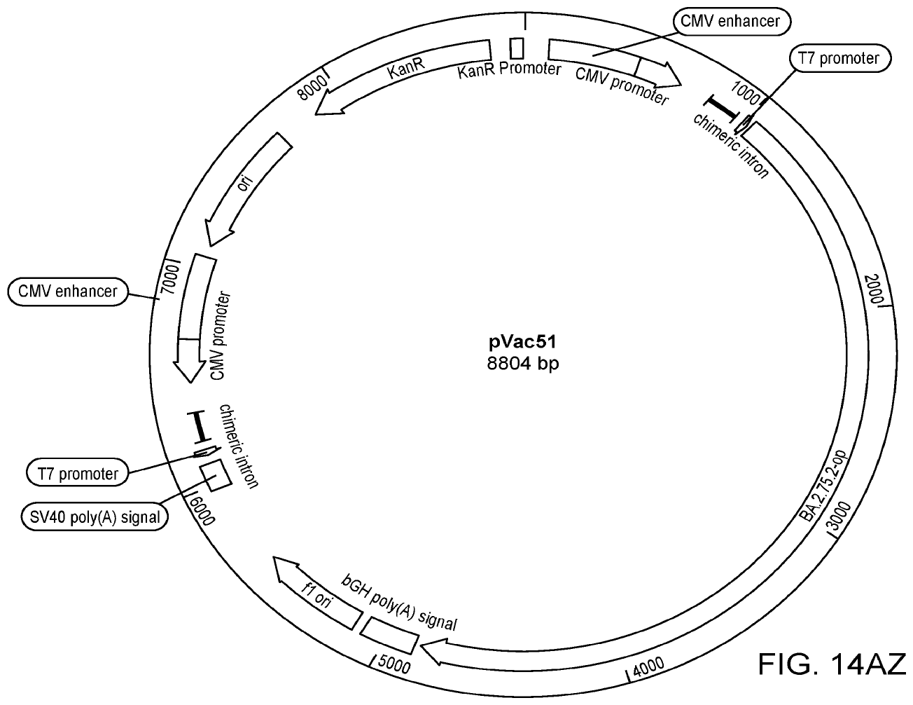

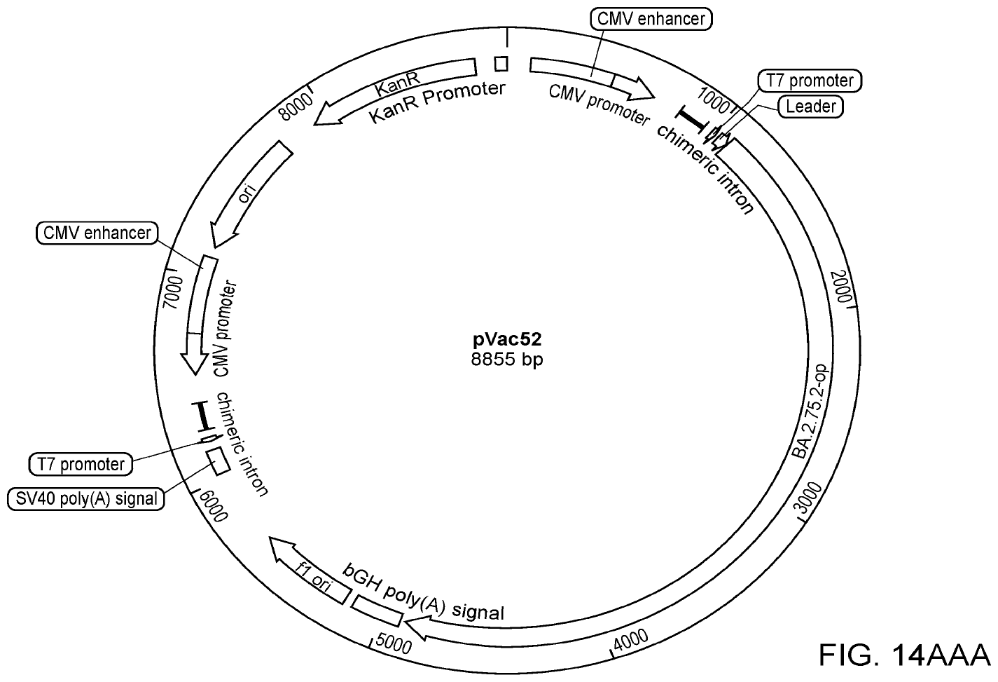

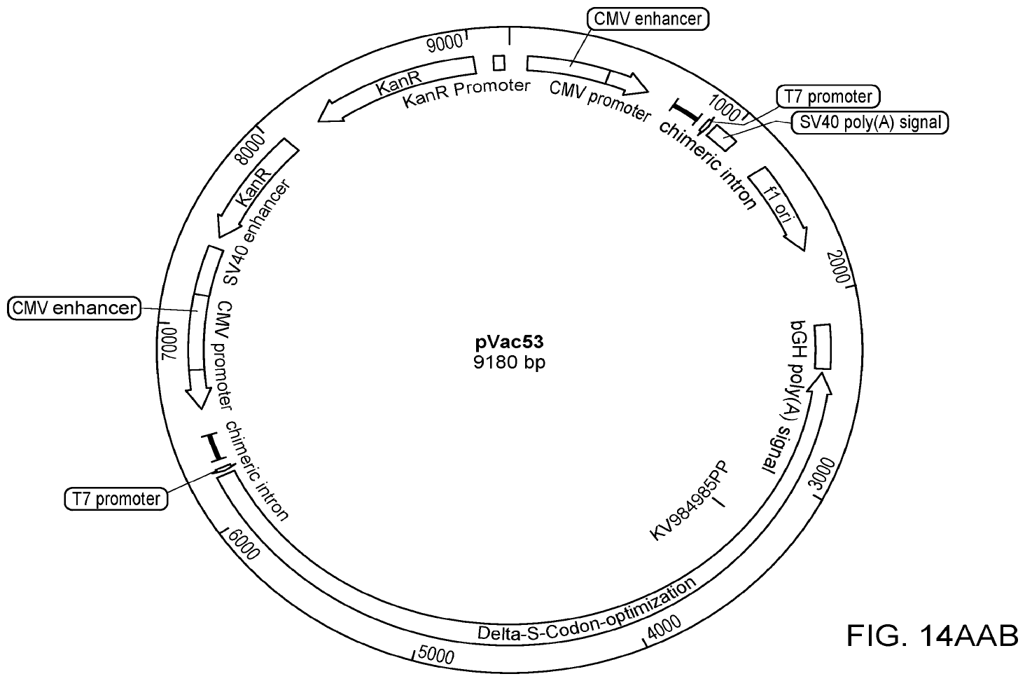

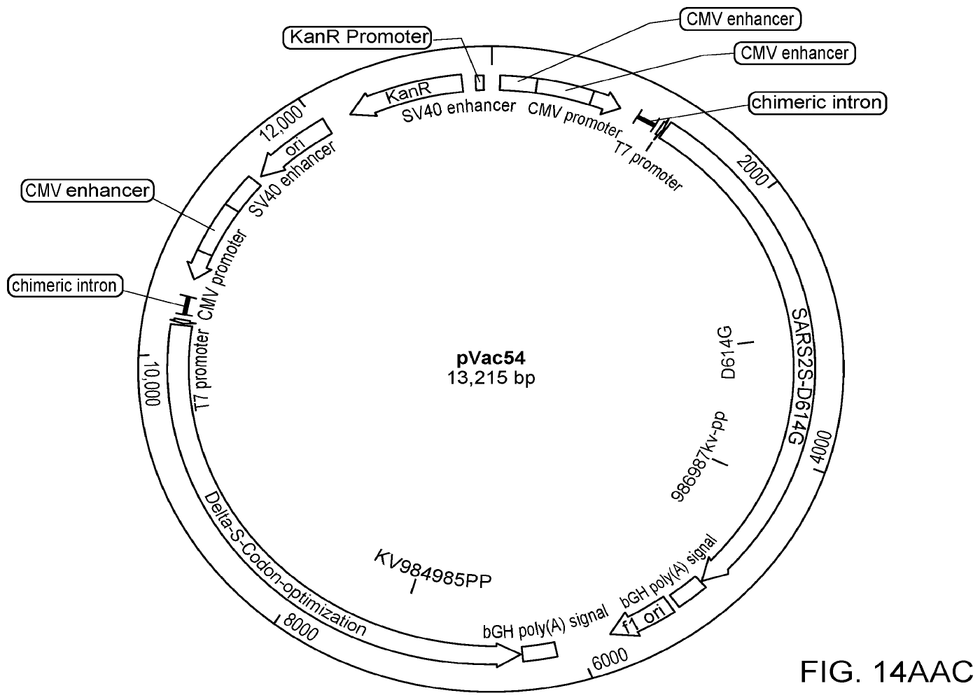

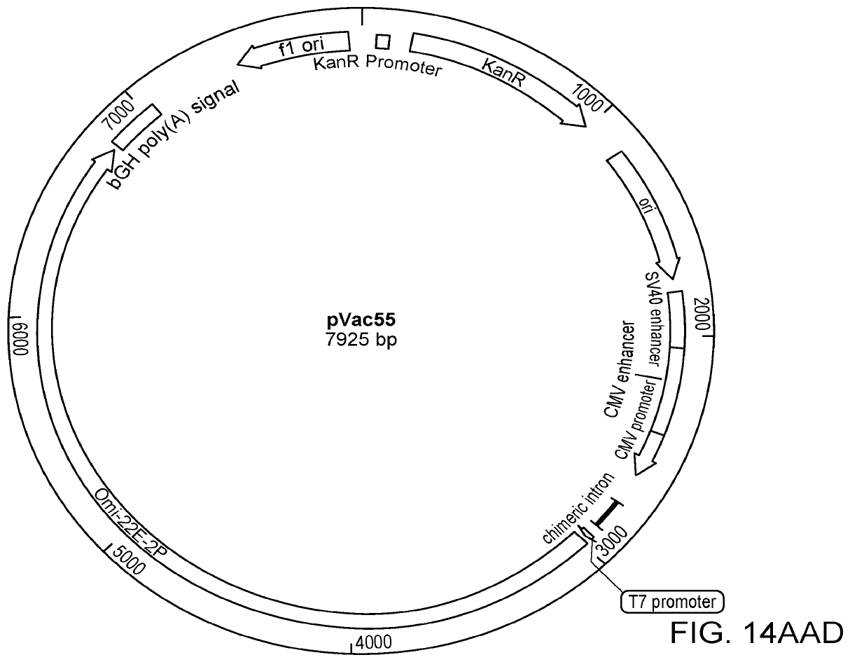

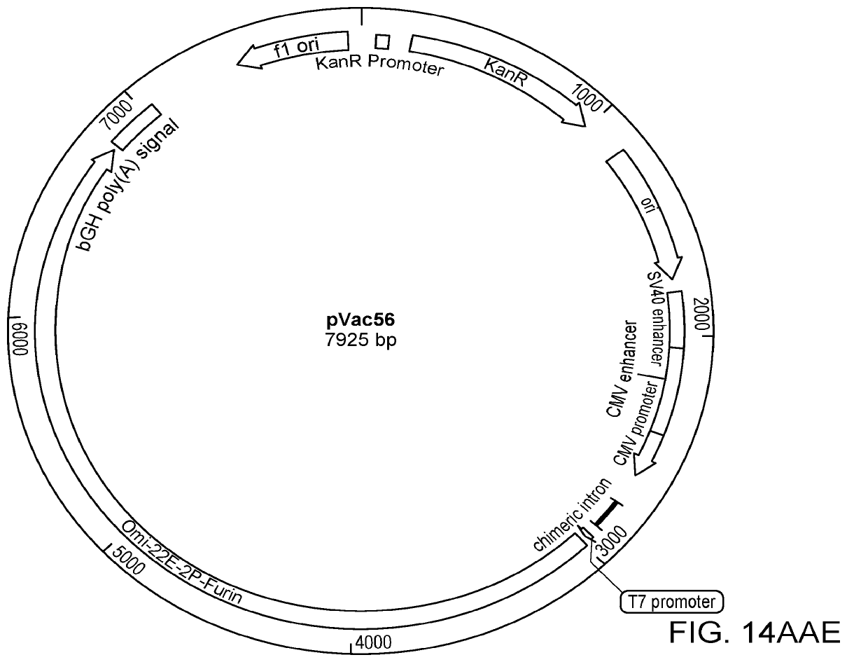

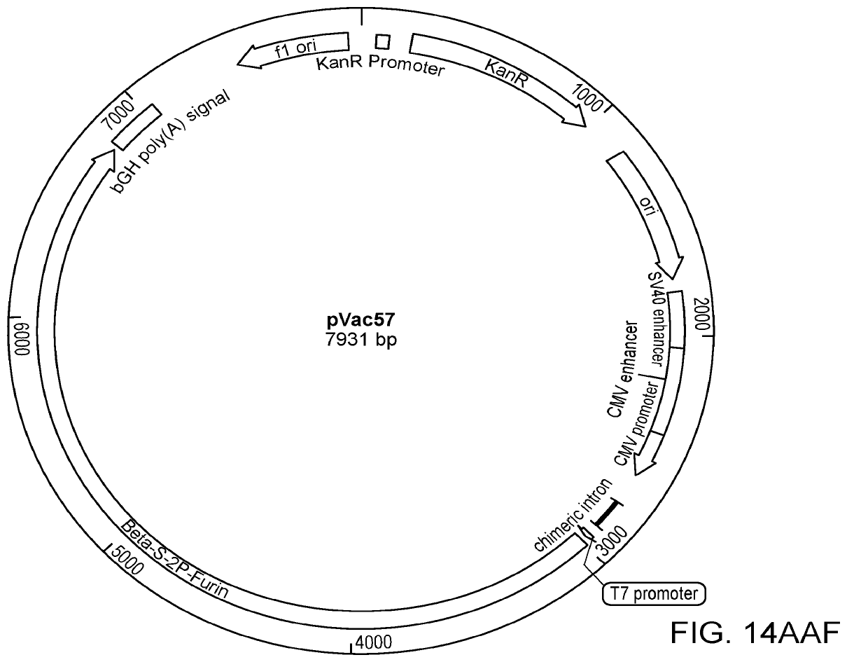

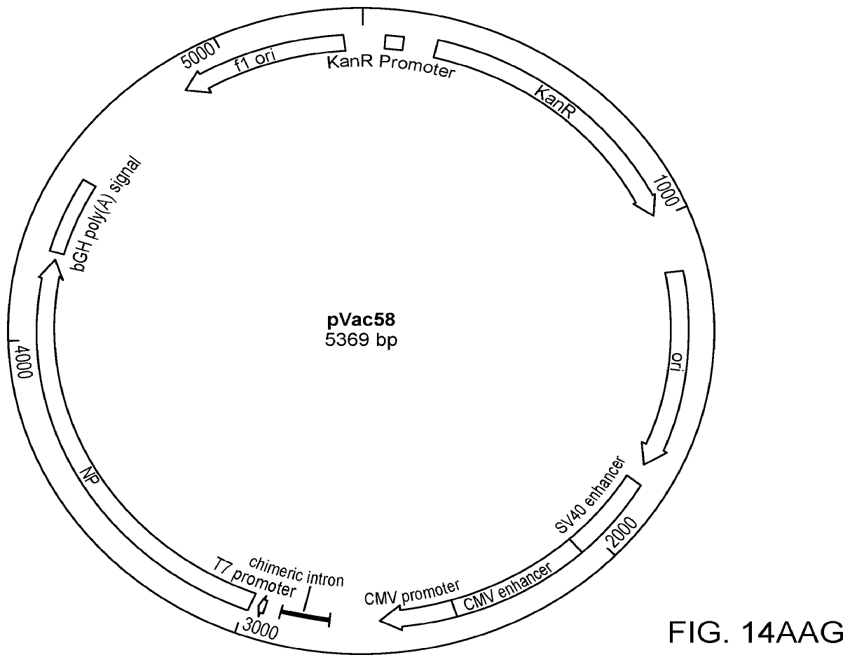

In some aspects, the polynucleotide (e.g., an expression vector) further comprises a fourth nucleic acid encoding a second immune modifier protein, wherein the fourth nucleic acid is operably linked to a fourth promoter. In some aspects, the polynucleotide can include the elements as disclosed in any of FIGS. 14B to 14AAK (pVac 1, pVac 2, pVac 3, pVac 4, pVac 5, pVac 6, pVac 7, pVac 8 pVac 9, pVac 10 pVac 11, pVac 12, pVac 13, pVac 14, pVac 15 pVac 16, pVac 17, pVac 18, pVac 19, pVac 20, pVac 21, pVac 22, pVac 23, pVac 24, pVac 25, pVac 26, pVac 27, pVac 28, pVac 29, pVac 30, pVac 31, pVac 32, pVac 33, pVac 34, pVac 35, pVac 36, pVac 37, pVac 38, pH1N1 Brisbane, pVac40, pVac42, pVac43, pVac44, pVac45, pVac46, pVac47, pVac48, pVac49, pVac50, pVac51, pVac52, pVac53, pVac54, pVac55, pVac56, pVac57, pVac58, pVac59, pVac60, pVac61, and pVac62 respectively). In some aspects, the vector constructs illustrated in any of 14B to 14AAK (pVac 1, pVac 2, pVac 3, pVac 4, pVac 5, pVac 6, pVac 7, pVac 8 pVac 9, pVac 10 pVac 11, pVac 12, pVac 13, pVac 14, pVac 15 pVac 16, pVac 17, pVac 18, pVac 19, pVac 20, pVac 21, pVac 22, pVac 23, pVac 24, pVac 25, pVac 26, pVac 27, pVac 28, pVac 29, pVac 30, pVac 31, pVac 32, pVac 33, pVac 34, pVac 35, pVac 36, pVac 37, pVac 38, pH1N1 Brisbane, pVac40, pVac42, pVac43, pVac44, pVac45, pVac46, pVac47, pVac48, pVac49, pVac50, pVac51, pVac52, pVac53, pVac54, pVac55, pVac56, pVac57, pVac58, pVac59, pVac60, pVac61, and pVac62) can be modified to replace the SARS-CoV-2 full-length surface (S) protein, the SARS-CoV-2 full-length D614G S protein, or the S1 subunit of the SARS-CoV-2 S protein (a first nucleotide sequence encoding a SARS-CoV-2 protein) and/or the SARS-CoV-2 membrane (M) protein (a second nucleic acid encoding a SARS-CoV-2 protein) with nucleotide sequences encoding any combinations of pathogen antigen or antigenic fragment thereof disclosed herein.

In some aspects, the polynucleotide (e.g., an expression vector) can comprise a first nucleic acid encoding a first pathogen antigen (e.g., SARS-CoV-2 spike (S) protein or an antigenic fragment thereof), wherein the first nucleic acid is operably linked to a first promoter. In some aspects, the polynucleotide further comprises a second nucleic acid encoding a second pathogen antigen (e.g., SARS-CoV-2 membrane (M) protein or an antigenic fragment thereof). In some aspects, the second nucleic acid is operably linked to the first promoter through an IRES sequence. In some aspects, the first pathogen antigen and the second pathogen antigen are SARS-CoV-2 antigens from different SARS CoV-2 strains. In some aspects, the first pathogen antigen and the second pathogen antigen are different variants of the same SARS-CoV-2 antigen, wherein the different variants of the same SARS-CoV-2 antigen are derived from different strains of SARS CoV-2. In some aspects, the first pathogen antigen and the second pathogen antigen are different variants of a SARS-CoV-2 S protein antigen, wherein the different variants of the SARS-CoV-2 S protein antigen are derived from different strains of SARS CoV-2. In some aspects, the first pathogen antigen and the second pathogen antigen are monkeypox antigens. In some aspects, the polynucleotide further comprises a second promoter, and the second nucleic acid is operably linked to the second promoter. In some aspects, the polynucleotide can include the elements as disclosed in any of FIGS. 14B (pVac 1), 14 E (pVac 4), or 14H (pVac 7). In some aspects, the vector constructs illustrated in any of FIGS. 14B to 14AAK (pVac 1, pVac 2, pVac 3, pVac 4, pVac 5, pVac 6, pVac 7, pVac 8 pVac 9, pVac 10 pVac 11, pVac 12, pVac 13, pVac 14, pVac 15 pVac 16, pVac 17, pVac 18, pVac 19, pVac 20, pVac 21, pVac 22, pVac 23, pVac 24, pVac 25, pVac 26, pVac 27, pVac 28, pVac 29, pVac 30, pVac 31, pVac 32, pVac 33, pVac 34, pVac 35, pVac 36, pVac 37, pVac 38, pH1N1 Brisbane, pVac40, pVac42, pVac43, pVac44, pVac45, pVac46, pVac47, pVac48, pVac49, pVac50, pVac51, pVac52, pVac53, pVac54, pVac55, pVac56, pVac57, pVac58, pVac59, pVac60, pVac61, and pVac62) can be modified to replace the SARS-CoV-2 full-length surface (S) protein, the SARS-CoV-2 full-length D614G S protein, or the S1 subunit of the SARS-CoV-2 S protein (a first nucleotide sequence encoding a SARS-CoV-2 protein) and/or the SARS-CoV-2 membrane (M) protein (a second nucleic acid encoding a SARS-CoV-2 protein) and/or the SARS-CoV-2 nucleocapsid (N) protein (a third nucleic acid encoding a SARS-CoV-2 protein) with nucleotide sequences encoding any combinations of pathogen antigen or antigenic fragment thereof disclosed herein.

In some aspects, the polynucleotide (e.g., an expression vector) can comprise a first nucleic acid encoding a first pathogen antigen (e.g., a first SARS-CoV-2 spike (S) protein or an antigenic fragment thereof), wherein the first nucleic acid is operably linked to a first promoter. In some aspects, the polynucleotide further comprises a second nucleic acid encoding a second pathogen antigen (e.g., a second SARS-CoV-2 spike (S) protein or an antigenic fragment thereof). In some aspects, the second nucleic acid is operably linked to the first promoter through an IRES sequence. In some aspects, the polynucleotide further comprises a second promoter, and the second nucleic acid is operably linked to the second promoter. In some aspects, the first pathogen antigen and the second pathogen antigen are SARS-CoV-2 antigens from different SARS CoV-2 strains. In some aspects, the first pathogen antigen and the second pathogen antigen are different variants of the same SARS-CoV-2 antigen, wherein the different variants of the same SARS-CoV-2 antigen are derived from different strains of SARS CoV-2. In some aspects, the first pathogen antigen and the second pathogen antigen are different variants of a SARS-CoV-2 S protein antigen, wherein the different variants of the SARS-CoV-2 S protein antigen are derived from different strains of SARS CoV-2. In some aspects, the first pathogen antigen and the second pathogen antigen are monkeypox antigens. In some aspects, the polynucleotide can include the elements as disclosed in any of FIGS. 14N, 145, 14T, 14U, 14Z, 14AA, 14AD, 14AF, 14AI, 14AL, 14AR, 14AT, 14AU, 14AX, 14AAC, and 14AAH (pVac12, pVac17, pVac18, pVac19, pVac24, pVac25, pVac28, pVac30, pVac33, pVac36, pVac43, pVac45, pVac46, pVac49, pVac54, and pVac59). In some aspects, the vector constructs illustrated in any of FIGS. 14N, 145, 14T, 14U, 14Z, 14AA, 14AD, 14AF, 14AI, 14AL, 14AR, 14AT, 14AU, 14AX, 14AAC, and 14AAH (pVac12, pVac17, pVac18, pVac19, pVac24, pVac25, pVac28, pVac30, pVac33, pVac36, pVac43, pVac45, pVac46, pVac49, pVac54, and pVac59) can be modified to replace the SARS-CoV-2 full-length surface (S) protein, the SARS-CoV-2 full-length D614G S protein, or the S1 subunit of the SARS-CoV-2 S protein with nucleotide sequences encoding any combinations of pathogen antigen or antigenic fragment thereof disclosed herein.

In some aspects, the polynucleotide (e.g., an expression vector) can comprise a first nucleic acid encoding a first pathogen antigen (e.g., a first SARS-CoV-2 spike (S) protein or an antigenic fragment thereof), wherein the first nucleic acid is operably linked to a first promoter. In some aspects, the polynucleotide further comprises a second nucleic acid encoding a second pathogen antigen (e.g., a second SARS-CoV-2 spike (S) protein or an antigenic fragment thereof). In some aspects, the polynucleotide further comprises a third nucleic acid sequence encoding a third pathogen antigen (e.g., a SARS-CoV-2 nucleocapsid (N) protein). In some aspects, the polynucleotide further comprises a second promoter, and the second nucleic acid is operably linked to the second promoter. In some aspects, the polynucleotide further comprises a third promoter, and the third nucleic acid is operably linked to the third promoter. In some aspects, the first pathogen antigen and the second pathogen antigen are SARS-CoV-2 antigens from different SARS CoV-2 strains. In some aspects, the first pathogen antigen and the second pathogen antigen are different variants of the same SARS-CoV-2 antigen, wherein the different variants of the same SARS-CoV-2 antigen are derived from different strains of SARS CoV-2. In some aspects, the first pathogen antigen and the second pathogen antigen are different variants of a SARS-CoV-2 S protein antigen, wherein the different variants of the SARS-CoV-2 S protein antigen are derived from different strains of SARS CoV-2. In some aspects, the first pathogen antigen and the second pathogen antigen are monkeypox antigens. In some aspects, the polynucleotide can include the elements as disclosed in any of FIGS. 14AAI and 14AAJ (pVac60 and pVac61). In some aspects, the vector constructs illustrated in any of FIGS. 14AAI and 14AAJ (pVac60 and pVac61) can be modified to replace the SARS-CoV-2 full-length surface (S) protein, the SARS-CoV-2 full-length D614G S protein, or the S1 subunit of the SARS-CoV-2 S protein with nucleotide sequences encoding any combinations of pathogen antigen or antigenic fragment thereof disclosed herein.

In some aspects, the composition comprises a polynucleotide (e.g., an expression vector) comprising: a nucleic acid encoding a pathogen antigen (e.g., Alpha SARS-CoV-2 strain spike (S) protein or an antigenic fragment thereof), wherein the nucleic acid is operably linked to a promoter. In some aspects, the composition comprises a polynucleotide (e.g., an expression vector) comprising: a nucleic acid encoding a pathogen antigen (e.g., Beta SARS-CoV-2 strain spike (S) protein or an antigenic fragment thereof), wherein the nucleic acid is operably linked to a promoter. In some aspects, the composition comprises a polynucleotide (e.g., an expression vector) comprising: a nucleic acid encoding a pathogen antigen (e.g., Gamma SARS-CoV-2 strain spike (S) protein or an antigenic fragment thereof), wherein the nucleic acid is operably linked to a promoter. In some aspects, the composition comprises a polynucleotide (e.g., an expression vector) comprising: a nucleic acid encoding a pathogen antigen (e.g., Delta SARS-CoV-2 strain spike (S) protein or an antigenic fragment thereof), wherein the nucleic acid is operably linked to a promoter. In some aspects, the composition comprises a polynucleotide (e.g., an expression vector) comprising: a nucleic acid encoding a pathogen antigen (e.g., Kappa SARS-CoV-2 strain spike (S) protein or an antigenic fragment thereof), wherein the nucleic acid is operably linked to a promoter. In some aspects, the composition comprises a polynucleotide (e.g., an expression vector) comprising: a nucleic acid encoding a pathogen antigen (e.g., Eta SARS-CoV-2 strain spike (S) protein or an antigenic fragment thereof), wherein the nucleic acid is operably linked to a promoter. In some aspects, the composition comprises a polynucleotide (e.g., an expression vector) comprising: a nucleic acid encoding a pathogen antigen (e.g., Iota SARS-CoV-2 strain spike (S) protein or an antigenic fragment thereof), wherein the nucleic acid is operably linked to a promoter. In some aspects, the composition comprises a polynucleotide (e.g., an expression vector) comprising: a nucleic acid encoding a pathogen antigen (e.g., Mu SARS-CoV-2 strain spike (S) protein or an antigenic fragment thereof), wherein the nucleic acid is operably linked to a promoter. In some aspects, the composition comprises a polynucleotide (e.g., an expression vector) comprising: a nucleic acid encoding a pathogen antigen (e.g., Epsilon SARS-CoV-2 strain spike (S) protein or an antigenic fragment thereof), wherein the nucleic acid is operably linked to a promoter. In some aspects, the composition comprises a polynucleotide (e.g., an expression vector) comprising: a nucleic acid encoding a pathogen antigen (e.g., Omicron SARS-CoV-2 strain spike (S) protein or an antigenic fragment thereof), wherein the nucleic acid is operably linked to a promoter. In some aspects, the composition comprises a polynucleotide (e.g., an expression vector) comprising: a nucleic acid encoding a pathogen antigen (e.g., monkeypox A35R protein or an antigenic fragment thereof), wherein the nucleic acid is operably linked to a promoter. In some aspects, the composition comprises a polynucleotide (e.g., an expression vector) comprising: a nucleic acid encoding a pathogen antigen (e.g., monkeypox H3L protein or an antigenic fragment thereof), wherein the nucleic acid is operably linked to a promoter. In some aspects, the composition comprises a polynucleotide (e.g., an expression vector) comprising: a nucleic acid encoding a pathogen antigen (e.g., monkeypox L1R protein or an antigenic fragment thereof), wherein the nucleic acid is operably linked to a promoter.

In some aspects, the pathogen antigen is a single pathogen antigen, a first pathogen antigen and/or the second pathogen antigen selected from a SARS-CoV-2 S protein or antigenic fragment thereof from an Alpha SARS-CoV-2 strain, wherein the SARS-CoV-2 S protein or antigenic fragment thereof comprises the amino acid sequence of SEQ ID NO: 2 or SEQ ID NO: 4 with one or more mutations selected from AH69-V70, A144, E484K, N501Y, A570D, D614G, P681H, T7161, S982A, and D1118H wherein the amino acid locations correspond to SEQ ID NO: 2 or SEQ ID NO: 4. In some aspects, the pathogen antigen is a single pathogen antigen, a first pathogen antigen and/or the second pathogen antigen selected from a SARS-CoV-2 S protein or antigenic fragment thereof from a SARS-CoV-2 S protein or antigenic fragment thereof from a Beta SARS-CoV-2 strain, wherein the SARS-CoV-2 S protein or antigenic fragment thereof comprises the amino acid sequence of SEQ ID NO: 2 SEQ ID NO: 4, SEQ ID NO: 125 with one or more mutations selected from L18F, D80A, D215G, AL241-S243, K417N, E484K, N501Y, D614G, and A701V, wherein the amino acid locations correspond to SEQ ID NO: 2 or SEQ ID NO: 4. In some aspects, the pathogen antigen is a single pathogen antigen, a first pathogen antigen and/or the second pathogen antigen selected from a SARS-CoV-2 S protein or antigenic fragment thereof from a SARS-CoV-2 S protein or antigenic fragment thereof from a Gamma SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, T20N, P26S, D138Y, R190S, K417T, E484K, N501Y, D614G, H655Y, T1027I, and V1176F, wherein the amino acid locations correspond to SEQ ID NO: 2 or SEQ ID NO: 4. In some aspects, the pathogen antigen is a single pathogen antigen, a first pathogen antigen and/or the second pathogen antigen selected from a SARS-CoV-2 S protein or antigenic fragment thereof from a Delta SARS-CoV-2 strain, wherein the SARS-CoV-2 S protein or antigenic fragment thereof comprises the amino acid sequence of SEQ ID NO: 2 or SEQ ID NO: 4 with one or more mutations selected from T19R, AD119-F120, AE156-F157, R158G, L452R, T478K, D614G, P681R, and D950N, wherein the amino acid locations correspond to SEQ ID NO: 2 or SEQ ID NO: 4. In some aspects, the pathogen antigen is a single pathogen antigen, a first pathogen antigen and/or the second pathogen antigen selected from a SARS-CoV-2 S protein or antigenic fragment thereof from a Kappa SARS-CoV-2 strain, wherein the SARS-CoV-2 S protein or antigenic fragment thereof comprises the amino acid sequence of SEQ ID NO: 2 or SEQ ID NO: 4 with one or more mutations selected from E154K, L452R, E484Q, D614G, P681R, and Q1071H, wherein the amino acid locations correspond to SEQ ID NO: 2 or SEQ ID NO: 4. In some aspects, the pathogen antigen is a single pathogen antigen, a first pathogen antigen and/or the second pathogen antigen selected from a SARS-CoV-2 S protein or antigenic fragment thereof from an Eta SARS-CoV-2 strain, wherein the SARS-CoV-2 S protein or antigenic fragment thereof comprises the amino acid sequence of SEQ ID NO: 2 or SEQ ID NO: 4 with one or more mutations selected from Q52R, A67V, AH69-V70, AY144, E484K, D614G, Q677H, and F888L, wherein the amino acid locations correspond to SEQ ID NO: 2 or SEQ ID NO: 4. In some aspects, the pathogen antigen is a single pathogen antigen, a first pathogen antigen and/or the second pathogen antigen selected from a SARS-CoV-2 S protein or antigenic fragment thereof from an Iota SARS-CoV-2 strain, wherein the SARS-CoV-2 S protein or antigenic fragment thereof comprises the amino acid sequence of SEQ ID NO: 2 or SEQ ID NO: 4 with one or more mutations selected from L5F, T95I, D253G, E484K, D614G, and A701V, wherein the amino acid locations correspond to SEQ ID NO: 2 or SEQ ID NO: 4. In some aspects, the pathogen antigen is a single pathogen antigen, a first pathogen antigen and/or the second pathogen antigen selected from a SARS-CoV-2 S protein or antigenic fragment thereof from a Lambda SARS-CoV-2 strain, wherein the SARS-CoV-2 S protein or antigenic fragment thereof comprises the amino acid sequence of SEQ ID NO: 2 or SEQ ID NO: 4 with one or more mutations selected from G75V, T76I, AR246-G252, D253N, L452Q, F490S, D614G, and T859N, wherein the amino acid locations correspond to SEQ ID NO: 2 or SEQ ID NO: 4. In some aspects, the pathogen antigen is a single pathogen antigen, a first pathogen antigen and/or the second pathogen antigen selected from a SARS-CoV-2 S protein or antigenic fragment thereof from a Mu SARS-CoV-2 strain, wherein the SARS-CoV-2 S protein or antigenic fragment thereof comprises the amino acid sequence of SEQ ID NO: 2 or SEQ ID NO: 4 with one or more mutations selected from T95I, Y144S, Y145N, R346K, E484K, N501Y, D614G, P681H, and D950N, wherein the amino acid locations correspond to SEQ ID NO: 2 or SEQ ID NO: 4. In some aspects, the pathogen antigen is a single pathogen antigen, a first pathogen antigen and/or the second pathogen antigen selected from a SARS-CoV-2 S protein or antigenic fragment thereof from an Epsilon SARS-CoV-2 strain, wherein the SARS-CoV-2 S protein or antigenic fragment thereof comprises the amino acid sequence of SEQ ID NO: 2 or SEQ ID NO: 4 with one or more mutations selected from S131, W152C, L452R, and D614G, wherein the amino acid locations correspond to SEQ ID NO: 2 or SEQ ID NO: 4.

In some aspects, the pathogen antigen, first pathogen antigen and/or the at least one additional pathogen antigen comprise a SARS-CoV-2 S protein or antigenic fragment thereof from an Omicron SARS-CoV-2 strain BA.1, wherein the SARS-CoV-2 S protein or antigenic fragment thereof comprises the amino acid sequence of SEQ ID NO: 2 or SEQ ID NO: 4 with one or more mutations selected from A67V, AH69-V70, T95I, G142-Y145D, N211I, A212I, 214EPEins, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, wherein the amino acid locations correspond to SEQ ID NO: 2 or SEQ ID NO: 4.

In some aspects, the pathogen antigen is a single pathogen antigen, a first pathogen antigen and/or the second pathogen antigen is selected from a SARS-CoV-2 S protein or antigenic fragment thereof from an Omicron SARS-CoV-2 strain BA.2, wherein the SARS-CoV-2 S protein or antigenic fragment thereof comprises the amino acid sequence of SEQ ID NO: 2 or SEQ ID NO: 4 with one or more mutations selected from T19I, LPPA24-27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K, wherein the amino acid locations correspond to SEQ ID NO: 2 or SEQ ID NO: 4.

In some aspects, the pathogen antigen, first pathogen antigen, the second pathogen antigen, the third pathogen antigen, and/or the at least one additional pathogen antigen comprise a SARS-CoV-2 S protein or antigenic fragment thereof from an Omicron SARS-CoV-2 strain BA.2.75.2, wherein the SARS-CoV-2 S protein or antigenic fragment thereof comprises the amino acid sequence of SEQ ID NO: 2 or SEQ ID NO: 4 with one or more mutations selected from T19I, AL24, AP25, AP26, A27S, G142D, K147E, W152R, F157L, I210V, V213G, G257S, G339H, S371F, S373P, S375F, T376A, D405N, R408S, K417N, N440K, G446S, N460K, S477N, T478K, E484A, R493Q, Q498R, N501Y, Y505H, D614G, H655Y, N679K, P681H, N764K, D796Y, Q954H, N969K, wherein the amino acid locations correspond to SEQ ID NO: 2 or SEQ ID NO: 4.

In some aspects, the pathogen antigen, first pathogen antigen, the second pathogen antigen, the third pathogen antigen, and/or the at least one additional pathogen antigen comprises an amino acid sequence having at least 70%, at least 75%, at least 80%, at least 85%, at least 90%, at least 95%, at least 99%, or 100% sequence identity to the amino acid sequence of SEQ ID NO: 115. In some aspects, the pathogen antigen, first pathogen antigen, the second pathogen antigen, the third pathogen antigen, and/or the at least one additional pathogen antigen comprises the amino acid sequence of SEQ ID NO: 115.

In some aspects, the pathogen antigen, first pathogen antigen, the second pathogen antigen, and/or the at least one additional pathogen antigen comprise a SARS-CoV-2 S protein or antigenic fragment thereof from an Omicron SARS-CoV-2 strain 22E (BQ.1), wherein the SARS-CoV-2 S protein or antigenic fragment thereof comprises the amino acid sequence of SEQ ID NO: 2 or SEQ ID NO: 4 with one or more mutations selected from T19I, AL24, AP25, AP26, A27S, AH69, AV70, G142D, V213G, G339D, S371F, S373P, S375F, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, N969K, wherein the amino acid locations correspond to SEQ ID NO: 2 or SEQ ID NO: 4.

In some aspects, the pathogen antigen, first pathogen antigen, the second pathogen antigen, the third pathogen antigen, and/or the at least one additional pathogen antigen comprises an amino acid sequence having at least 70%, at least 75%, at least 80%, at least 85%, at least 90%, at least 95%, at least 99%, or 100% sequence identity to the amino acid sequence of SEQ ID NO: 117. In some aspects, the pathogen antigen, first pathogen antigen, the second pathogen antigen, the third pathogen antigen, and/or the at least one additional pathogen antigen comprises the amino acid sequence of SEQ ID NO: 117.

In some aspects, the pathogen antigen, first pathogen antigen, the second pathogen antigen, and/or the at least one additional pathogen antigen comprise a SARS-CoV-2 S protein or antigenic fragment thereof from an Omicron SARS-CoV-2 strain BA.5, wherein the SARS-CoV-2 S protein or antigenic fragment thereof comprises the amino acid sequence of SEQ ID NO: 2 or SEQ ID NO: 4 with one or more mutations selected from T19I, AL24, AP25, AP26, A27S, AH69, AV70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K, wherein the amino acid locations correspond to SEQ ID NO: 2 or SEQ ID NO: 4.

In some aspects, the pathogen antigen, first pathogen antigen, the second pathogen antigen, the third pathogen antigen, and/or the at least one additional pathogen antigen comprises an amino acid sequence having at least 70%, at least 75%, at least 80%, at least 85%, at least 90%, at least 95%, at least 99%, or 100% sequence identity to the amino acid sequence of SEQ ID NO: 121. In some aspects, the pathogen antigen, first pathogen antigen, the second pathogen antigen, the third pathogen antigen, and/or the at least one additional pathogen antigen comprises the amino acid sequence of SEQ ID NO: 121.

In some aspects, the pathogen antigen, first pathogen antigen, the second pathogen antigen, and/or the at least one additional pathogen antigen comprise a SARS-CoV-2 nucleocapsid (N) protein or antigenic fragment thereof from an Omicron SARS-CoV-2 strain BQ.1 (22E), wherein the SARS-CoV-2 N protein or antigenic fragment thereof comprises the amino acid sequence of SEQ ID NO: 28 with one or more mutations selected from P13L, A31, A32, A33, E136D, R203K, G204R, S413R, wherein the amino acid locations correspond to SEQ ID NO: 28.

In some aspects, the pathogen antigen, first pathogen antigen, the second pathogen antigen, the third pathogen antigen, and/or the at least one additional pathogen antigen comprises an amino acid sequence having at least 70%, at least 75%, at least 80%, at least 85%, at least 90%, at least 95%, at least 99%, or 100% sequence identity to the amino acid sequence of SEQ ID NO: 123. In some aspects, the pathogen antigen, first pathogen antigen, the second pathogen antigen, the third pathogen antigen, and/or the at least one additional pathogen antigen comprises the amino acid sequence of SEQ ID NO: 123.

In some aspects, the pathogen antigen, first pathogen antigen, the second pathogen antigen, the third pathogen antigen, and/or the at least one additional pathogen antigen comprise a SARS-CoV-2 S protein or antigenic fragment thereof from an Omicron SARS-CoV-2 strain BA.XBB1.5, wherein the SARS-CoV-2 S protein or antigenic fragment thereof comprises the amino acid sequence of SEQ ID NO: 2 or SEQ ID NO: 4 with one or more mutations selected from T19I, AL24, AP25, AP26, A27S, V83A, G142D, AY144, H146Q, Q183E, V213E, G252V, G339H, R346T, L3681, S371F, S373P, S375F, T376A, D405N, R408S, K417N, N440K, V445P, G446S, N460K, S477N, T478K, E484A, F486P, F490S, R493Q, Q498R, N501Y, Y505H, D614G, H655Y, N679K, P681H, N764K, D796Y, Q954H, N969K, wherein the amino acid locations correspond to SEQ ID NO: 2 or SEQ ID NO: 4.

In some aspects, the pathogen antigen, first pathogen antigen, the second pathogen antigen, the third pathogen antigen, and/or the at least one additional pathogen antigen comprises an amino acid sequence having at least 70%, at least 75%, at least 80%, at least 85%, at least 90%, at least 95%, at least 99%, or 100% sequence identity to the amino acid sequence of SEQ ID NO: 125. In some aspects, the pathogen antigen, first pathogen antigen, the second pathogen antigen, the third pathogen antigen, and/or the at least one additional pathogen antigen comprises the amino acid sequence of SEQ ID NO: 125.

In some aspects, the pathogen antigen is a single pathogen antigen, a first pathogen antigen, the second pathogen antigen, or the third pathogen antigen, comprising a nucleic acid sequence having at least 70%, at least 75%, at least 80%, at least 85%, at least 90%, at least 95%, at least 99%, or 100% sequence identity to the nucleic acid sequence of SEQ ID NO: 114, SEQ ID NO: 116, SEQ ID NO: 118, SEQ ID NO: 120, and SEQ ID NO: 124. In some aspects, the pathogen antigen is a single pathogen antigen, a first pathogen antigen, the second pathogen antigen, or the third pathogen antigen comprising a nucleic acid sequence of any of SEQ ID NO: 114, SEQ ID NO: 116, SEQ ID NO: 118, SEQ ID NO: 120, SEQ ID NO: 122, and SEQ ID NO: 124.

In some aspects, the pathogen antigen is a single pathogen antigen, a first pathogen antigen, the second pathogen antigen, or the third pathogen antigen comprising an amino acid sequence having at least 70%, at least 75%, at least 80%, at least 85%, at least 90%, at least 95%, at least 99%, or 100% sequence identity to the amino acid sequence of SEQ ID NO: 72, SEQ ID NO: 73, SEQ ID NO: 115, SEQ ID NO: 117, SEQ ID NO: 119, SEQ ID NO: 121, SEQ ID NO: 123, and SEQ ID NO: 125. In some aspects, the pathogen antigen is a single pathogen antigen, a first pathogen antigen, the second pathogen antigen, or the third pathogen antigen comprising an amino acid sequence of any of SEQ ID NO: 72, SEQ ID NO: 73, SEQ ID NO: 115, SEQ ID NO: 117, SEQ ID NO: 119, SEQ ID NO: 121, and SEQ ID NO: 125.

In some aspects, the pathogen antigen is a single pathogen antigen, a first pathogen antigen, the second pathogen antigen, or the third pathogen antigen comprising a nucleic acid sequence having at least 70%, at least 75%, at least 80%, at least 85%, at least 90%, at least 95%, at least 99%, or 100% sequence identity to the nucleic acid sequence of SEQ ID NO: 126. In some aspects, the pathogen antigen is a single pathogen antigen, a first pathogen antigen, the second pathogen antigen, or the third pathogen antigen comprising a nucleic acid sequence of SEQ ID NO: 127.

In some aspects, the pathogen antigen is a single pathogen antigen, a first pathogen antigen, the second pathogen antigen, or the third pathogen antigen comprising a nucleic acid sequence having at least 70%, at least 75%, at least 80%, at least 85%, at least 90%, at least 95%, at least 99%, or 100% sequence identity to the nucleic acid sequence of SEQ ID NO: 126. In some aspects, the pathogen antigen is a single pathogen antigen, a first pathogen antigen, the second pathogen antigen, or the third pathogen antigen comprising a nucleic acid sequence of SEQ ID NO: 127.

In some aspects, the pathogen antigen is a single pathogen antigen, a first pathogen antigen and/or the second pathogen antigen comprises an amino acid sequence of any of SEQ ID NO: 67, SEQ ID NO: 69, SEQ ID NO: 71, SEQ ID NO: 72, SEQ ID NO: 73, SEQ ID NO: 74, SEQ ID NO: 75, SEQ ID NO: 76, SEQ ID NO: 77 SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, or SEQ ID NO: 113.

In some aspects, the pathogen antigen is a single pathogen antigen, a first pathogen antigen and/or the second pathogen antigen comprising the amino acid sequence having at least 70%, at least 75%, at least 80%, at least 85%, at least 90%, at least 95%, at least 99%, or 100% sequence identity to the amino acid sequence of SEQ ID NO: 67, SEQ ID NO: 69, SEQ ID NO: 71, SEQ ID NO: 72, SEQ ID NO: 73, SEQ ID NO: 74, SEQ ID NO: 75, SEQ ID NO: 76, SEQ ID NO: 77 SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, or SEQ ID NO: 113.

In some aspects, the compositions, vaccines, or pharmaceutical compositions further comprise a delivery component (e.g., a cationic polymer such as a biodegradable cross-linked cationic multi-block copolymer, a PEG-PEI-cholesterol (PPC) lipopolymer, a lipopolyamine, or a lipopolyamine derivative). In some aspects, the PEG-PEI-cholesterol (PPC) lipopolymer has an average PEG:PEI:cholesterol ratio of 2.5:1:0.6.

In some aspects, the delivery component exhibits an adjuvant property. Not to be bound by any theory, in some aspects, the adjuvant property of the delivery component promotes mobilization of antigen presenting cells to the site of vaccine delivery and antigen expression, thereby augmenting the uptake of the polynucleotide and the expressed antigens into professional antigen presenting cells to elicit MHC Class I and MHC Class II presentation.

In some aspects, the present disclosure is directed to vaccines or compositions comprising (i) a vector (e.g., a multicistronic DNA plasmid vector or a multicistronic messenger RNA (mRNA) vector) comprising a nucleic acid sequence encoding one or more viral antigens (e.g., a SARS CoV-2 antigen or a monkeypox antigen), (ii) a delivery component (e.g., a cationic polymer, a poly-inosinic-polycytidylic acid, or a poloxamer), (iii) a STING agonist, and (iv) an ALUM adjuvant.

In some aspects, the present disclosure is directed to vaccines or compositions comprising (i) a vector (e.g., a multicistronic DNA plasmid vector or a multicistronic messenger RNA (mRNA) vector) comprising a nucleic acid sequence encoding one or more viral antigens (e.g., a SARS CoV-2 antigen or a monkeypox antigen), (ii) a delivery component (e.g., a cationic polymer, a poly-inosinic-polycytidylic acid, or a poloxamer), and (iii) a STING agonist.

In some aspects, the present disclosure is directed to vaccines or compositions comprising (i) a vector (e.g., a multicistronic DNA plasmid vector or a multicistronic messenger RNA (mRNA) vector) comprising a nucleic acid sequence encoding one or more viral antigens (e.g., a SARS CoV-2 antigen or a monkeypox antigen), (ii) a delivery component (e.g., a cationic polymer, a poly-inosinic-polycytidylic acid, or a poloxamer), and (iii) an ALUM adjuvant.

In some aspects, the vector further comprises a nucleic acid sequence encoding one or more immune modifier proteins. In some aspects, the immune modifier protein is selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the nucleic acid encoding the at least one immune modifier protein is operably linked to a promoter.

In some aspects, the vector comprises a nucleic acid sequence encoding a SARS CoV-2 antigen and, optionally, a second viral antigen. In some aspects, the vector comprises a nucleic acid sequence encoding a SARS CoV-2 antigen and a second SARS CoV-2 antigen from a different SARS CoV-2 strain. In some aspects, the vector comprises a nucleic acid sequence encoding a SARS CoV-2 S protein antigen and a second SARS CoV-2 S protein antigen from a different SARS CoV-2 strain.

In some aspects, the vector comprises a nucleic acid sequence encoding a monkeypox antigen and, optionally, a second viral antigen. In some aspects, the vector comprises a nucleic acid sequence encoding a monkeypox antigen and a second monkeypox antigen from a different monkeypox strain.

Some aspects relate to methods of eliciting humoral and/or cellular immune response against a pathogen (e.g., SARS-CoV-2 or monkeypox) challenge or infection following in vivo administration of a vector or composition of the disclosure.

The present disclosure is also directed to methods of inducing an immune response in a subject comprising administering an effective amount of any composition, pharmaceutical composition, vaccine, polynucleotide, or vector disclosed herein to the subject. The present disclosure is also directed to methods of preventing, reducing the incidence of, attenuating or treating a viral, a bacterial or a parasite infection in a subject comprising administering an effective amount of any composition, polynucleotide, vector, pharmaceutical composition, or vaccine disclosed herein to the subject. In some aspects, the infection is a SARS-CoV-2 viral infection. In some aspects, the infection is a monkeypox viral infection. The present disclosure is also directed to methods of making any composition, pharmaceutical composition, or vaccine disclosed herein.

### II. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein, the term “approximately” or “about,” as applied to one or more values of interest, refers to a value that is similar to a stated reference value and within a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). When the term “approximately” or “about” is applied herein to a particular value, the value without the term “approximately” or “about is also disclosed herein.

As described herein, any concentration range, percentage range, ratio range, or integer range is to be understood to include the value of any integer within the recited range and, when appropriate, fractions thereof (such as one tenth and one hundredth of an integer), unless otherwise indicated.

As used herein, the terms “ug” and “uM” are used interchangeably with “μg” and “μM,” respectively.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As used herein, the term “coronavirus” or “CoV” refers to the common name for Coronaviridae. In humans, CoV causes respiratory infections, which are typically mild but can be lethal in rare forms such as SARS (severe acute respiratory syndrome)-CoV, MERS (Middle East Respiratory Syndrome)-CoV, and SARS-CoV-2. CoV has a nucleocapsid of helical symmetry and the genome size ranges from about 26 to about 32 kilobases. Other exemplary human CoV include CoV 229E, CoV NL63, CoV OC43, CoV HKU1, and CoV HKU20. The envelope of CoV carries three glycoproteins: spike (S) protein (receptor binding, cell fusion, major antigen); envelope (E) protein (small, envelope-associated protein); and membrane (M) protein (budding and envelope formation). In a few types of CoV, there is a fourth glycoprotein: hemagglutinin-esterase (HE) protein. The genome has a 5′ methylated cap and 3′ poly-A and functions directly as mRNA. Entry of the CoV into a human cell occurs via endocytosis and membrane fusion; and replication occurs in the cell's cytoplasm. CoV are transmitted by aerosols of respiratory secretions, by the faecal-oral route, and by mechanical transmission. Most virus growth occurs in epithelial cells. Occasionally, the liver, kidneys, heart, or eyes can be infected, as well as other cell types such as macrophages.

As used herein, the term “SARS-CoV-2” refers to the strain of coronavirus that causes coronavirus disease 2019 (COVID-19), the respiratory illness responsible for the COVID-19 pandemic. Taxonomically, SARS-CoV-2 is a member of the subgenus Sarbecovirus (beta-CoV lineage B) and is a strain of SARS-CoV. It is believed to have zoonotic origins and has close genetic similarity to bat coronaviruses, suggesting it emerged from a bat-borne virus. Its RNA sequence is approximately 30,000 bases in length. SARS-CoV-2 is unique among known betacoronaviruses in its incorporation of a polybasic cleavage site, a characteristic known to increase pathogenicity and transmissibility in other viruses. Like other coronaviruses, SARS-CoV-2 has four structural proteins, the S (spike), E (envelope), M (membrane), and N (nucleocapsid) proteins. The N protein holds the RNA genome, and the S, E, and M proteins together create the viral envelope. The spike protein is the protein responsible for allowing the virus to attach to and fuse with the membrane of a host cell; specifically, its S1 subunit catalyzes attachment, the S2 subunit fusion. Protein modeling experiments on the spike protein of the virus have suggested that SARS-CoV-2 has sufficient affinity to the receptor angiotensin converting enzyme 2 (ACE2) on human cells to use them as a mechanism of cell entry. See Xu, X, et al., Science China Life Sciences, 63(3):457-60 (2020). SARS-CoV-2 can also use basigin to assist in cell entry. See Wang, K., et al., bioRxiv, doi:10.1101/2020.03.14.988345 (2020).

The terms “nucleic acids,” “nucleic acid molecules, “nucleotides,” “nucleotide(s) sequence,” and “polynucleotide” can be used interchangeably and refer to the phosphate ester polymeric form of ribonucleosides (adenosine, guanosine, uridine or cytidine; “RNA molecules”, including mRNA) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, DNA plasmid, synthetic DNA, and semi-synthetic DNA. A “nucleic acid composition” of the disclosure comprises one or more nucleic acids as described herein.

RNA can be obtained by transcription of a DNA-sequence, e.g., inside a cell. In eukaryotic cells, transcription is typically performed inside the nucleus or the mitochondria. In vivo, transcription of DNA usually results in premature RNA, which has to be processed into messenger RNA (mRNA). Processing of the premature RNA, e.g., in eukaryotic organisms, comprises a variety of different posttranscriptional-modifications such as splicing, 5′-capping, polyadenylation, export from the nucleus or the mitochondria and the like. The sum of these processes is also called maturation of RNA. The mature mRNA usually provides the nucleotide sequence that can be translated into an amino acid sequence of a particular peptide, protein, or protein antigen. Typically a mature mRNA comprises a 5′ cap, optionally a 5′-UTR, an open reading frame, optionally a 3′-UTR, and a poly(A) sequence.

The term “multicistronic mRNA” or “multicistronic mRNA vector,” as used herein, refers to an mRNA having two or more open reading frames. An open reading frame in this context is a sequence of codons that is translatable into a polypeptide or protein.

The term “5′-cap,” as used herein, refers to an entity, typically a modified nucleotide entity, which generally “caps” the 5′-end of a mature mRNA. A 5′-cap can typically be formed by a modified nucleotide, particularly by a derivative of a guanine nucleotide. In some aspects, the 5′-cap is linked to the 5′-terminus via a 5′-5′-triphosphate linkage. A 5′-cap can be methylated, e.g., m7GpppN, wherein N is the terminal 5′ nucleotide of the nucleic acid carrying the 5′-cap, typically the 5′-end of an RNA. The naturally occurring 5′-cap is m7GpppN.

As used herein, a “poly(A) sequence,” also called “poly(A) tail” or “3′-poly(A) tail,” is typically understood to be a sequence of adenine nucleotides, e.g., of up to about 400 adenine nucleotides. A poly(A) sequence can be located at the 3′ end of an mRNA. In some aspects, a poly(A) sequence can also be located within an mRNA or any other nucleic acid molecule, such as, e.g., in a vector, for example, in a vector serving as template for the generation of an RNA, preferably an mRNA, e.g., by transcription of the vector. In some aspects, a poly(A) sequence is present in the 3′-UTR of the mRNA as defined herein.

In some aspects, a 3′-UTR sequence is part of an mRNA, which is located between the protein coding region (i.e. the open reading frame) and the 3′ terminus of the mRNA molecule. If a 3′-terminal poly(A) sequence (‘poly(A) tail’) was added to the RNA (e.g. by polyadenylation), then the term 3′-UTR can refer to that part of the molecule, which is located between the protein coding region and the 3′-terminal poly(A) sequence. In some aspects, a 3′-UTR can also comprise a poly(A) sequence (e.g., a poly(A) sequence which is not located at the very 3′ terminus of the RNA molecule). A 3′-UTR of the mRNA is not translated into an amino acid sequence. The 3′-UTR sequence is generally encoded by the gene, which is transcribed into the respective mRNA during the gene expression process. The genomic sequence is first transcribed into pre-mature mRNA, which comprises optional introns. The pre-mature mRNA is then further processed into mature mRNA in a maturation process. This maturation process comprises the steps of 5′ capping, splicing the pre-mature mRNA to excise optional introns and modifications of the 3′-end, such as polyadenylation of the 3′-end of the pre-mature mRNA and optional endo-/or exonuclease cleavages etc. In some aspects, a 3′-UTR corresponds to the sequence of a mature mRNA, which is located 3′ to the stop codon of the protein coding region (e.g., immediately 3′ to the stop codon of the protein coding region), and which extends to the 3′ terminus of the RNA molecule or to the 5′-side of a 3′ terminal poly(A) sequence (e.g., to the nucleotide immediately 5′ to the 3′ terminus or immediately 5′ to the 3′ terminal poly(A) sequence). The term “corresponds to” means that the 3′-UTR sequence can be an RNA sequence, such as in the mRNA sequence used for defining the 3′-UTR sequence, or a DNA sequence, which corresponds to such RNA sequence. In some aspects, the term “a 3′-UTR of a gene”, such as “3′-UTR of alpha or beta globin”, is the sequence, which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the RNA sequence of the 3′-UTR. In some aspects, the 3′-UTR is derived from a gene that relates to an mRNA with an enhanced half-like (i.e., that provides a stable mRNA), for example a 3′-UTR of a gene selected from the group consisting of: albumin gene, an α-globin gene, a β-globin gene, a tyrosine hydroxylase gene, a lipoxygenase gene, and a collagen alpha gene, such as a collagen alpha 1(I) gene.

A 5′-UTR is typically understood to be a particular section of messenger RNA (mRNA). It is located 5′ of the open reading frame of the mRNA. In some aspect, the 5′-UTR starts with the transcriptional start site and ends one nucleotide before the start codon of the open reading frame. The 5′-UTR can comprise elements for controlling gene expression, also called regulatory elements. Such regulatory elements can be, for example, ribosomal binding sites or a 5′-Terminal Oligopyrimidine Tract. The 5′-UTR can be posttranscriptionally modified, for example by addition of a 5′-cap. In some aspects, a 5′-UTR corresponds to the sequence of a mature mRNA which is located between the 5′ cap and the start codon. In some aspects, the 5′-UTR corresponds to the sequence which extends from a nucleotide located 3′ to the 5′-cap (e.g., from the nucleotide located immediately 3′ to the 5′cap) to a nucleotide located 5′ to the start codon of the protein coding region (e.g., to the nucleotide located immediately 5′ to the start codon of the protein coding region). The nucleotide located immediately 3′ to the 5′ cap of a mature mRNA typically corresponds to the transcriptional start site. The term “corresponds to” means that the 5′-UTR sequence can be an RNA sequence, such as in the mRNA sequence used for defining the 5′-UTR sequence, or a DNA sequence which corresponds to such RNA sequence. In some aspects, the term “a 5′-UTR of a gene”, is the sequence, which corresponds to the 5′-UTR of the mature mRNA derived from this gene.

As used herein, the term “transfecting” or “transfection” refers to the transport of nucleic acids from the environment external to a cell to the internal cellular environment, with particular reference to the cytoplasm and/or cell nucleus. Without being bound by any particular theory, it is to be understood that nucleic acids can be delivered to cells either after being encapsulated within or adhering to one or more cationic polymer/nucleic acid complexes or being entrained therewith. Particular transfecting instances deliver a nucleic acid to a cell nucleus. Nucleic acids include DNA and RNA as well as synthetic congeners thereof. Such nucleic acids include missense, antisense, nonsense, as well as protein producing nucleotides, on and off and rate regulatory nucleotides that control protein, peptide, and nucleic acid production. In particular, but not limited to, they can be genomic DNA, cDNA, mRNA, tRNA, rRNA, hybrid sequences or synthetic or semi-synthetic sequences, and of natural or artificial origin. In addition, the nucleic acid can be variable in size, ranging from oligonucleotides to chromosomes. These nucleic acids can be of human, animal, vegetable, bacterial, viral, or synthetic origin. They can be obtained by any technique known to a person skilled in the art.

As used herein, the term “biodegradable” or “biodegradation” is defined as the conversion of materials into less complex intermediates or end products by solubilization hydrolysis, or by the action of biologically formed entities which can be enzymes and other products of the organism.

As used herein, “peptide” means peptides of any length and includes proteins. The terms “polypeptide” and “oligopeptide” are used herein without any particular intended size limitation, unless a particular size is otherwise stated.

As used herein, a “derivative” of a carbohydrate includes, for example, an acid form of a sugar, e.g. glucuronic acid; an amine of a sugar, e.g. galactosamine; a phosphate of a sugar, e.g. mannose-6-phosphate; and the like.

As used herein, the term “inverted terminal repeat” (or “ITR”) refers to a single stranded sequence of nucleotides followed downstream by its reverse complement. The intervening sequence of nucleotides between the initial sequence and the reverse complement can be any length including zero.

“Administering” and similar terms refer to the physical introduction of a therapeutic agent (e.g., nucleic acid molecules, vectors, compositions, and pharmaceutical compositions described herein) to a subject, using any of the various methods and delivery systems known to those skilled in the art. Exemplary routes of administration include intravenous, intramuscular, intraarterial, intrathecal, intralymphatic, intralesional, intracapsular, intraorbital, intracardiac, intradermal, transdermal, intraperitoneal, transtracheal, subcutaneous, subcuticular, intraarticular, subcapsular, subarachnoid, intraspinal, epidural, intrasterna, oral, rectal, topical, epidermal, mucosal, intranasal, vaginal, rectal, sublingual administration, and combinations thereof. Administering can also be performed, for example, once, a plurality of times, and/or over one or more extended periods.

“Treatment” or “therapy” of a subject refers to any type of intervention or process performed on, or the administration of an active agent to, a subject with the objective of reversing, alleviating, ameliorating, inhibiting, slowing down, or preventing the onset, progression, development, severity, or recurrence of a symptom, complication, condition, or biochemical indicia associated with a disease.

A “therapeutically effective amount,” “effective amount,” “therapeutic dose,” “effective dose,” or “effective dosage,” as used herein, means an amount or a dose that achieves a therapeutic goal, as described herein. One of ordinary skill in the art will further understand that a therapeutically effective amount etc. can be administered in a single dose, or can be achieved by administration of multiple doses (i.e., 1, 2, 3, 4, 5, 6, 7, 8, 9, 10 or more doses). The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition.

As used herein, the term “adjuvant” refers to any component which improves the body's response to a vaccine.

As used herein, the term “ALUM” or “Alum” as use herein refers to an aluminum or aluminum-salt. In some aspects, the ALUM is an aluminum or aluminum-salt based adjuvant, e.g., aluminum phosphate, aluminum hydroxide, potassium aluminum sulfate [KAl(SO₄)₂], aluminumcrystalline aluminum oxyhydroxide, aluminum hydroxyphosphate, amorphous aluminum hydroxyphosphate sulfate, aluminum chloride, aluminum silicate, and a mixture of aluminum hydroxide and magnesium hydroxide, a mixture of aluminum sulfate and sodium hydroxide or a mixture of aluminum sulfate and potassium hydroxide. An ALUM can be an aluminum hydroxide gel or aluminum phosphate gel.

As used herein, the term “STING” refers to the stimulator of interferon genes protein that is a pattern recognition receptor (PRR) that senses cyclic dinucleotides and induces the expression of type I interferon canonically via dendritic cells.

As used herein, the term “STING agonist” refers to a molecule that induces a STING pathway.

As used herein, the terms “vaccine” or “vaccine composition” refer to an immunogenically active composition for the prophylaxis and/or treatment of diseases. Accordingly, in some aspects, vaccines are medicaments which comprise or deliver antigens and are intended to be used in humans or animals for generating specific defense and protective substance by vaccination

A used herein, the term “inducing immunity” or “immunogenically active” refers to the ability to stimulate an immune response, i.e., to stimulate the production of antibodies, particularly humoral antibodies, or to stimulate a cell-mediated response. For example, the ability to stimulate the production of circulating or secretory antibodies or the production of a cell-mediated response in local mucosal regions, peripheral blood, cerebral spinal fluid or the like. In some aspects, the effective immunizing amount of the immunogenically active component(s) of this disclosure can vary and can be any amount sufficient to evoke an immune response and provide a protective immune response against SARS-CoV-2 virus infection or monkeypox virus infection. A dosage unit comprising a polynucleotide (e.g., plasmid DNA) of the disclosure is contemplated. At least one dosage unit per patient is contemplated herein as a vaccination regimen. In some embodiments, two or more dosage units can be useful. The skilled artisan will quickly recognize that a particular quantity of vaccine composition per dosage unit, as well as the total number of dosage units per vaccination regimen, can be optimized, so long as an effective immunizing amount of the virus or a component thereof is ultimately delivered to the subject.

An “immunological response” to a substance such as a composition or vaccine is the development in the subject of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. In some aspects, the subject can display either a therapeutic or protective immunological response so resistance to new infection will be enhanced and/or the clinical severity of the disease reduced. In some aspects, such protection can be demonstrated by either a reduction or lack of symptoms normally displayed by an infected subject, a quicker recovery time and/or a lowered viral titer in the infected subject.

It is recognized that the antigenic polypeptides of the disclosure can be full length polypeptides or active fragments or variants thereof. In some aspects, the term “active fragments” or “active variants” or “antigenic fragments” refers to fragments or variants that retain all or some of the antigenic nature of the polypeptide. Thus, in some aspects, the present disclosure encompasses any SARS-CoV-2 polypeptide, antigen, epitope or immunogen that elicits an immunogenic response in a subject. The SARS-CoV-2 polypeptide, antigen, epitope or immunogen can be any SARS-CoV-2 polypeptide, antigen, epitope or immunogen, such as, but not limited to, a protein, peptide or fragment or variant thereof, that elicits, induces or stimulates a response in a subject. The SARS-CoV-2 polypeptide, antigen, epitope or immunogen can be derived from any strain of SARS-CoV-2 including, but not limited to, an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); a Zeta strain (e.g., strain P.2), or an Omicron SARS-CoV-2 stain (e.g., strain B.1.1.529, BA.1 BA.2, BA.5, BA.2.75.2, BQ.1, XBB1.5, or 22E). Mutations and viral sequence data for the SARS-CoV-2 variants are publically available at the CoVariants website (https://covariants.org/) and the National Center for Biotechnology Information (NCBI) website (https://www.ncbi.nlm.nih.gov/labs/virus/vssi/#/sars-cov-2), each of which is incorporated by reference in its entirety.

The term “epitope” refers to the site on an antigen or hapten to which specific B cells and/or T cells respond. The term is also used interchangeably with “antigenic determinant” or “antigenic determinant site.” Antibodies that recognize the same epitope can be identified in a simple immunoassay showing the ability of one antibody to block the binding of another antibody to a target antigen.

As used herein, the term “pharmaceutical agent,” “pharmaceutical composition,” or “drug” or any other similar term means any chemical or biological material or compound suitable for administration by the methods previously known in the art and/or by the methods taught in the present disclosure, which induce a desired biological or pharmacological effect, which can include but are not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating a disease from the organism. The effect can be local or it can be systemic.

A “pharmaceutically acceptable carrier” refers to a carrier that can be administered to a subject, together with an agent, and which does not destroy the pharmacological activity thereof and is nontoxic when administered in doses sufficient to deliver a therapeutic amount of the agent. In certain aspects, the pharmaceutically acceptable carrier is an aqueous solvent, i.e., a solvent comprising water, optionally with additional co-solvents. Exemplary pharmaceutically acceptable carriers include water, buffer solutions in water (such as phosphate-buffered saline (PBS), and 5% dextrose in water (D5W). In certain embodiments, the aqueous solvent further comprises dimethyl sulfoxide (DMSO), e.g., in an amount of about 1-4%, or 1-3%. In certain aspects, the pharmaceutically acceptable carrier is isotonic (i.e., has substantially the same osmotic pressure as a body fluid such as plasma).

A “subject” includes any human or non-human animal. The term “nonhuman animal” includes, but is not limited to, vertebrates such as nonhuman primates, sheep, dogs, and rodents such as mice, rats, and guinea pigs. In some aspects, the subject is a human. The terms “subject” and “patient” are used interchangeably herein.

The term “expression” as used herein refers to a process by which a polynucleotide produces a gene product, for example, a SARS-CoV-2 S protein, a monkeypox A35R protein, a monkeypox H3L protein, a monkeypox L1R protein, or antigenic fragment thereof. In some aspects, it includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein can further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

As used herein, the term “5′” or “5 prime” refers to the 5′ end of a nucleic acid or nucleic acid sequence, and the term “3′” or “3 prime” refer to the 3′ end of nucleic acid or nucleic acid sequence.

The terms “identical” or percent “identity” in the context of two or more nucleic acids refer to two or more sequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

As used herein, the term “promoter” refers to DNA sequence capable of controlling the expression of a coding sequence or functional RNA. In some aspects, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The term “operably linked” refers to genetic elements that are joined together in a manner that enables them to carry out their normal functions. For example, a gene is operably linked to a promoter when its transcription is under the control of the promoter and this transcription results in the production of the product encoded by the gene.

As used herein, the term “immune modifier protein” refers to a protein that augments the immune response to a one or more antigens. In some aspects, immune modifier proteins include, but are not limited to, a cytokine, a chemokine, major histocompatibility complex (MHC) class I (MHC I), MHC class II (MHC II), human leukocyte antigen (HLA)-DR isotype (HLA-DR), CD80, CD86, and any combination thereof. Cytokine immune modifier proteins include, but are not limited to, interleukin (IL) 2 (IL-2), IL-12 p35, IL-12 p40, IL-12 p70, IL-15, IL-18, tumor necrosis factor alpha (TNFα), granulocyte-macrophage colony-stimulating factor (GM-CSF), interferon (IFN) a (IFN-α), and IFN-β. Chemokine immune modifier proteins include, but are not limited to, C—C motif chemokine ligand (CCL) 3 (CCL3), CCL4, CCL5, CCL21, CCL28, C—X—C motif chemokine ligand (CXCL) 10 (CXCL10), and any combination thereof. In some aspects, the immune modifier proteins include a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the immune modifier proteins include SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, and any combination thereof. In some aspects, the immune modifier proteins include one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety. In some aspects, an immune modifier protein as disclosed herein can include a combination of immune modifier proteins, e.g., a cytokine or chemokine protein or their coding sequence either co-expressed from the same plasmid as the antigen or from a different plasmid. See, e.g., Hirao, L.A., et al, Vaccine, 26:3112-20 (2008); Kanagavelu, S. K., et al., Vaccine, 30:691-702 (2012); Ahlers, D. J., et al., Curr Mol Med., 3:285-301 (2003); Jafarfzade, B. S., et al., Bratisl Lek Listy, 118:564-9 (2017); Ahler, J. D. and Belyakov, I.M., Eur J Immunol, 39:2657-69 (2009); Moore, A. C., et al., J Virol. 76:243-50 (2002); Barouch, D. H., et al., PNAS, 97:4192-7 (2000); Kalams, S. A., et al., J Infectious Disease, 208:818-29 (2013); Buchbinder, S., et al., PLOS One, 12(7):e0179597 (2017); Henke, A, et al., Intervirology, 49:249-52 (2006); and Yang, S. H., et al., Gen Ther., 13:1110-7 (2006).

The term “targeting ligand,” as used herein, is intended to refer to ligands conjugated to a polymer either directly or via one or more spacer molecules. In some aspects, only a small portion of the available amino groups of the polymer is coupled to the ligand. In some aspects, the targeting ligands conjugated to the polymers direct the polymers-nucleic acid complex to bind to specific target cells and penetrate into such cells (e.g., epithelial cells, endothelial cells, hematopoietic cells, and the like). In some aspects, the target ligands can also be an intracellular targeting element, enabling the transfer of the nucleic acid/drug to be guided towards certain favored cellular compartments (mitochondria, nucleus, and the like). In some aspects, the ligand is a polypeptide, folate, and an antigen. In some aspects, the polypeptide ligand is a glycoprotein (e.g., transferrin or asialoorosomucoid (ASOR)), an antibody, an antibody fragment, a cell receptor, a cytokine receptor, or a growth factor receptor (e.g., epidermal growth factor receptor). In some aspects, the antigen ligand is a viral antigen, a bacterial antigen, or a parasite antigen. In some aspects, the ligand is a fusogenic agent (e.g., polymixin B and hemaglutinin HA2), a lysosomotrophic agent, or a nucleus localization signal (NLS) (e.g., T-antigen, and the like). In some aspects, the ligand is a sugar moiety coupled to an amino group. In some aspects, the sugar moiety is a mono- or oligo-saccharide, such as galactose, glucose, fucose, fructose, lactose, sucrose, mannose, cellobiose, nytrose, triose, dextrose, trehalose, maltose, galactosamine, glucosamine, galacturonic acid, glucuronic acid, and gluconic acid.

The term “antibody” includes molecules or active fragments (i.e., antigen binding fragments) of molecules that bind to antigens. These active fragments can be derived from an antibody of the present disclosure by a number of techniques. For further description of general techniques for the isolation of active fragments of antibodies, see for example, Khaw, B. A. et al. J. Nucl. Med. 23:1011-1019 (1982). The term “antibody” also includes bispecific and chimeric antibodies and antibodies in nonmammalian species.

The term “biodegradable linker” or “biofunctional biodegradable linker,” as used herein, refers to a biodegradable linker containing ester, amide, disulfide, and/or phosphate linkages that is used to cross-link cationic multi-block copolymers. In some aspects, the biodegradable linker is hydrophilic and comprises a biodegradable linkage comprising a disulfide bond. In some aspects, the biodegradable linker is a dithiodipropionyl linker.

The term “vector,” as used herein, is intended to refer to a nucleic acid molecule capable of transporting another nucleic acid to which it has been linked; or an entity comprising such a nucleic acid molecule capable of transporting another nucleic acid. In some aspects, the vector is a “plasmid,” which refers to a circular double stranded DNA loop into which additional DNA segments can be ligated. In some aspects, the vector is a viral vector, wherein additional DNA segments can be ligated into the viral genome. In some aspects, such vectors include, but are not limited to: an adenoviral vector, an adeno-associated virus (AAV) vector, retroviral vector, a lentiviral vector, poxvirus vector, a baculovirus vector, a herpes viral vector, simian virus 40 (SV40), cytomegalovirus (CMV), mouse mammary tumor virus (MMTV), and Moloney murine leukemia virus. Certain vectors, or polynucleotides that are part of vectors, are capable of autonomous replication in a host cell into which they are introduced (e.g., bacterial vectors having a bacterial origin of replication, and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can sometimes be used interchangeably, depending on the context, as the plasmid is the most commonly used form of vector. However, also disclosed herein are other forms of expression vectors, such as viral vectors (e.g., replication defective retroviruses, poxviruses, herpesviruses, baculoviruses, adenoviruses and adeno-associated viruses), which can serve equivalent functions.

As used herein, the term “poloxamer” or “poloxamer backbone” refers to molecules having the general formula HO—(C₂H₄O)ₐ(C₃H₆O)_{b}(C₂H₄O)_{c}—H in which a and c are approximately equal. See, Handbook of Biodegradable Polymers, Chapter 12′ “The Poloxamers: Their Chemistry and Medical Applications” authored by Lorraine E. Reeve. See also U.S. Publ. No. 2010/0004313, which is herein incorporated by reference in its entirety. Because the poloxamers are the products of a sequential series of reactions, the chain lengths of individual poloxamer blocks are statistical distributions about the average chain length. Thus, the number of ethyleneoxy groups (a and c) and the number of propylenoxy groups (b) are meant to be averages. Poloxamers are generally based on an amphiphilic triblock copolymer of ethylene oxide and propylene oxide, having a central hydrophobic chain of polypropylene oxide flanked by two hydrophilic chains of polyethylene oxide. Because the lengths of the polymer blocks of a poloxamer backbone can vary between various polymeric constructs, many different poloxamers are considered to be within the scope of the present disclosure. In one aspect, for example, the average molecular weight of the poloxamer backbone can range from about 100 to about 100,000 Dalton. In another aspect, the average molecular weight of the poloxamer backbone can range from about 500 to about 50,000 Dalton. In yet another aspect, the average molecular weight of the poloxamer backbone can range from about 1000 to about 20,000 Dalton. The poloxamer backbone can also be described in terms of a ratio of ethylene oxide to propylene oxide. In another aspect, the ratio of ethylene oxide to propylene eoxide is from about 20:1 to about 1:20. For example, in one aspect the ratio of ethylene oxide to propylene oxide is from about 5:1 to about 1:5.

As used herein, “BD15-12” refers to a PEI-based co-block polymer that utilized biodegradable linkages.

Various additional aspects of the disclosure are described, disclosed or illustrated in further detail in the following subsections.

### III. Compositions

In some aspects, provided are compositions (e.g., pharmaceutical compositions or vaccines). In some aspects, the compositions comprise a polynucleotide as disclosed herein and an adjuvant. In some aspects, the compositions comprise a polynucleotide and a STING agonist. In some aspects, the compositions comprise a polynucleotide, an adjuvant, and a STING agonist. In some aspects, the compositions further comprise a delivery component.

In some aspects, the compositions (e.g., pharmaceutical compositions or vaccines) of the disclosure comprise a polynucleotide as described herein, an adjuvant, and a delivery component.

In some aspects, the compositions (e.g., pharmaceutical compositions or vaccines) of the disclosure comprise a polynucleotide as described herein, a STING agonist, and a delivery component.

In some aspects, the compositions (e.g., pharmaceutical compositions or vaccines) of the disclosure comprise a polynucleotide as described herein, an adjuvant, a STING agonist, and a delivery component.

In some aspects, the adjuvant is selected from potassium aluminum sulfate [KAl(SO₄)₂], aluminum hydroxide, crystalline aluminum oxyhydroxide, aluminum phosphate, aluminum hydroxyphosphate, amorphous aluminum hydroxyphosphate sulfate, aluminum chloride, aluminum silicate, and a composition comprising aluminum hydroxide and magnesium hydroxide.

In some aspects, the aluminum adjuvant is an aluminum hydroxide gel or aluminum phosphate gel.

In some aspects, the aluminum adjuvant is a mixture of aluminum sulfate and sodium or potassium hydroxide.

In some aspects, the immune stimulator is a STING agonist.

In some aspects, the STING agonist is cyclic GMP (cGMP). In some aspects, the STING agonist is cyclic AMP (cAMP). In some aspects, the STING agonist is cyclic-GMP-AMP (cGAMP).

In some aspects, the STING agonist is an amidobenzimidazole (ABZI).

In some aspects, the STING agonist is a cyclic dinucleotide selected from ADU-S100, MK-1454, SB11285, BMS-986301, BI-STING (B11387446), JNJ-67544412, 3′3′-cyclic AIMIP, and GSK532.

In some aspects, the STING agonist is a non-cyclic dinucleotide small molecule selected from DMAXAA, ALG-031048, E7766, JNJ-′6196, MK-21118, MSA-1, MSA-2, SNX281, SR-717, TAK676, TTI-10001, a Ryvu's agonist, GF3-002, a Selvita agonist, CDR5500, CS-1010, CS-1018, CS-1020, and [2-ex] MSA-1.

In some aspects, the STING agonist is a nanovaccine selected from PC7A nanoparticles, cGMP-nanoparticles, and ONM-500 nanoparticles.

In some aspects, the STING agonist is an antibody-drug conjugate. In some aspects, the antibody drug conjugate is XMT-2056.

In some aspects, the STING agonist is an ENPP1 inhibitor selected from MV-626, SR-8314, SR-8291, and SR8541A.

In some aspects, the STING agonist is a bacterial vector. In some aspects, the bacterial vector is a nonpathogenic E. coli nissle expressing cyclic-di-AMP-producing enzymes. In some aspects, the bacterial vector is SYNB1981. In some aspect, the bacterial vector is an attenuated Salmonella Typhimurium strain engineered to carry an inhibitory TREX-1 micro RNA. Also provided herein are compositions (e.g., pharmaceutical compositions and vaccines) comprising any polynucleotide or vector described or exemplified herein. In some aspects, the compositions (e.g., pharmaceutical composition or vaccine) further comprises a pharmaceutically acceptable carrier.

In some aspects, the compositions (e.g., pharmaceutical composition or vaccine) further comprises a second polynucleotide. In some aspects, the second polynucleotide encodes a second antigen wherein the second antigen is operably linked to a promoter. In some aspects, the second polynucleotide encodes at least one viral antigen (e.g., a SARS CoV-2 antigen). In some aspects, the second polynucleotide encodes at least one immune modifier protein. In some aspects, the immune modifier protein is selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the nucleic acid encoding the at least one immune modifier protein is operably linked to a promoter. In some aspects, the compositions (e.g., pharmaceutical composition or vaccine) further comprises a delivery component (e.g., a cationic polymer, a poly-inosinic-polycytidylic acid, or a poloxamer). In some aspects, the delivery component further comprises benzalkonium chloride.

In some aspects, the compositions (e.g., pharmaceutical composition or vaccine) further comprises a third polynucleotide. In some aspects, the third polynucleotide encodes a third antigen wherein the third antigen is operably linked to a promoter. In some aspects, the third polynucleotide encodes at least one viral antigen (e.g., a SARS CoV-2 antigen). In some aspects, the third polynucleotide encodes at least one immune modifier protein selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the nucleic acid encoding the at least one immune modifier protein is operably linked to a promoter. In some aspects, the compositions (e.g., pharmaceutical composition or vaccine) further comprises a delivery component (e.g., a cationic polymer, a poly-inosinic-polycytidylic acid, or a poloxamer). In some aspects, the delivery component further comprises benzalkonium chloride.

In some aspects, the compositions (e.g., pharmaceutical composition or vaccine) further comprises a fourth polynucleotide. In some aspects, the fourth polynucleotide encodes a fourth antigen wherein the fourth antigen is operably linked to a promoter. In some aspects, the fourth polynucleotide encodes at least one viral antigen (e.g., a SARS CoV-2 antigen). In some aspects, the fourth polynucleotide encodes at least one immune modifier protein selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the nucleic acid encoding the at least one immune modifier protein is operably linked to a promoter. In some aspects, the compositions (e.g., pharmaceutical composition or vaccine) further comprises a delivery component (e.g., a cationic polymer, a poly-inosinic-polycytidylic acid, or a poloxamer). In some aspects, the delivery component further comprises benzalkonium chloride.

The compositions (e.g., pharmaceutical compositions and vaccines) can comprise any polynucleotide or vector described or exemplified herein. In some aspects, the compositions (e.g., pharmaceutical composition or vaccine) further comprises a pharmaceutically acceptable carrier.

In some aspects, the at least one immune modifier protein encoded by the second, third, and/or fourth polynucleotide comprises a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, the viral protein is from a different virus than a viral antigen encoded by an antigen nucleic acid. In some aspects, the viral protein attenuates a local inflammatory response and/or interferon response elicited by a pathogen antigen disclosed herein.

In some aspects, the at least one immune modifier protein comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety. Also provided herein are compositions (e.g., pharmaceutical compositions or vaccines) comprising any polynucleotide, multicistronic mRNA vector, or DNA plasmid vector described or exemplified herein.

Certain aspects of the disclosure relate to compositions comprising (i) a vector (e.g., an expression vector, a multicistronic DNA plasmid vector or a multicistronic messenger RNA (mRNA) vector) comprising a nucleic acid sequence encoding one or more viral antigens (e.g., a SARS CoV-2 antigen or monkeypox antigen); (ii) a delivery component (e.g., a cationic polymer, a poly-inosinic-polycytidylic acid, or a poloxamer); and (iii) an adjuvant comprising an aluminum or aluminum-salt based adjuvant, a stimulator of interferon genes (STING) agonist, or a combination thereof. In some aspects, the adjuvant comprises an aluminum or aluminum-salt based adjuvant and a STING agonist. In some aspects, the aluminum or aluminum-salt based adjuvant comprises an aluminum oxyhydroxide, an aluminum hydroxide, an aluminum phosphate, or any combination thereof. In some aspects, the STING agonist is cyclic guanosine monophosphate (cGMP), cyclic adenosine monophosphate (cAMP), or cyclic guanosine monophosphate-adenosine monophosphate (cGAMP).

In some aspects, the vector further comprises a nucleic acid sequence encoding one or more immune modifier proteins. In some aspects, the vector comprises a nucleic acid sequence encoding a SARS CoV-2 antigen and, optionally, a second viral antigen. In some aspects, the vector comprises a nucleic acid sequence encoding a monkeypox antigen and, optionally, a second viral antigen. In some aspects, the vector comprises a first and second nucleic acid sequence encoding a first and second SARS CoV-2 antigen and, optionally, a third viral antigen. In some aspects, the vector comprises a first and second nucleic acid sequence encoding a first and second monkeypox antigen and, optionally, a third viral antigen. Some aspects relate to methods of eliciting humoral and/or cellular immune response against a pathogen (e.g., SARS-CoV-2 or monkeypox) challenge or infection following in vivo administration of a vector or composition of the disclosure.

Some aspects relate to methods of eliciting humoral and/or cellular immune response against a pathogen (e.g., SARS-CoV-2 or monkeypox) challenge or infection following in vivo administration of a multicistronic DNA plasmid, a multicistronic mRNA vector, or a composition comprising the same of the disclosure.

Certain aspects of the disclosure relate to compositions including (i) a DNA plasmid vector comprising a DNA sequence of one or more antigens; (ii) a delivery component, such as a synthetic non-viral DNA carrier (e.g., a cationic polymer, a poly-inosinic-polycytidylic acid, or a poloxamer); and (iii) an adjuvant comprising an aluminum or aluminum-salt based adjuvant, a stimulator of interferon genes (STING) agonist, or a combination thereof.

The delivery component of the compositions disclosed herein may comprise any combination of delivery components disclosed herein.

In some aspects, the compositions (e.g., pharmaceutical compositions or vaccines) of the disclosure comprise a delivery component and a polynucleotide comprising: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 1.

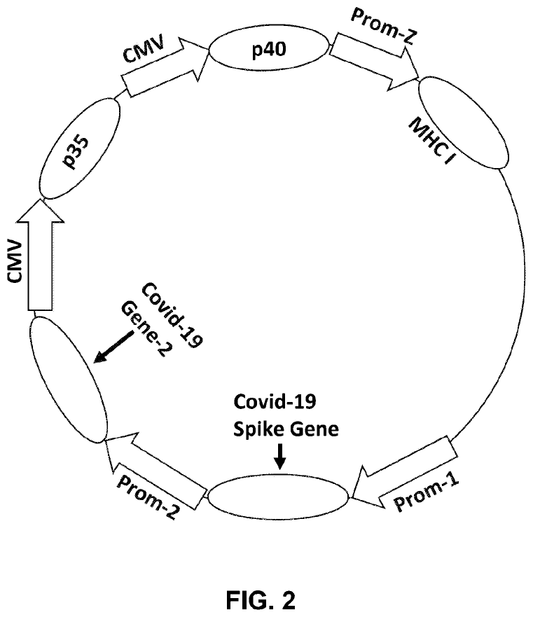

In some aspects, the compositions (e.g., pharmaceutical compositions or vaccines) of the disclosure comprise a delivery component and a polynucleotide comprising: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes MHC I and is operably linked to promoter Z; a fourth nucleotide sequence, wherein the fourth nucleotide sequence encodes a first SARS-CoV-2 protein and is operably linked to promoter 1, and a fifth nucleotide sequence, wherein the fifth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes MHC I and is operably linked to promoter Z; a fourth nucleotide sequence positioned 3′ to the third nucleotide sequence, wherein the fourth nucleotide sequence encodes a first SARS-CoV-2 protein and is operably linked to promoter 1, and a fifth nucleotide sequence positioned 3′ to the fourth nucleotide sequence, wherein the fifth nucleotide sequence encodes a second SARS-CoV-2 protein and is operably linked to promoter 2. In some aspects, the first, second, third, fourth, and fifth nucleotide sequences of the polynucleotide are configured as shown in FIG. 2.

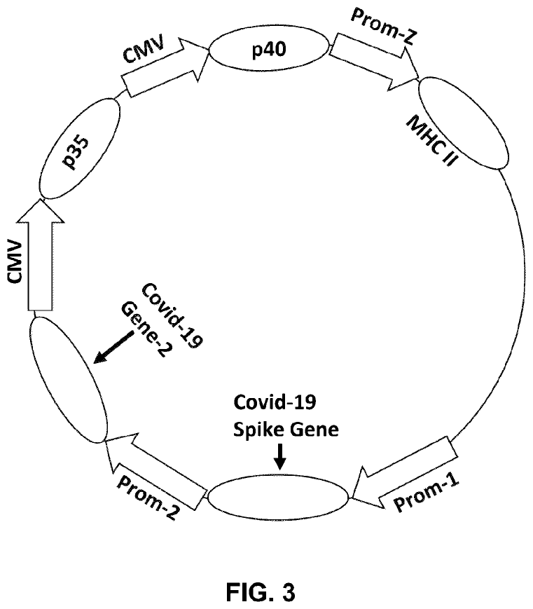

In some aspects, the compositions (e.g., pharmaceutical compositions or vaccines) of the disclosure comprise a delivery component and a polynucleotide comprising: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes MHC II and is operably linked to promoter Z; a fourth nucleotide sequence, wherein the fourth nucleotide sequence encodes a first SARS-CoV-2 protein and is operably linked to promoter 1, and a fifth nucleotide sequence, wherein the fifth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes MHC II and is operably linked to promoter Z; a fourth nucleotide sequence positioned 3′ to the third nucleotide sequence, wherein the fourth nucleotide sequence encodes a first SARS-CoV-2 protein and is operably linked to promoter 1, and a fifth nucleotide sequence positioned 3′ to the fourth nucleotide sequence, wherein the fifth nucleotide sequence encodes a second SARS-CoV-2 protein and is operably linked to promoter 2. In some aspects, the first, second, third, fourth, and fifth nucleotide sequences of the polynucleotide are configured as shown in FIG. 3.

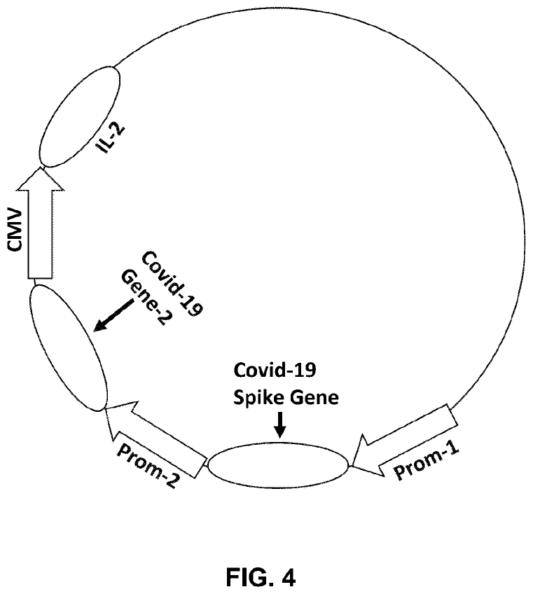

In some aspects, the compositions (e.g., pharmaceutical compositions or vaccines) of the disclosure comprise a delivery component and a polynucleotide comprising: a first nucleotide sequence, wherein the first nucleotide sequence encodes IL-2 and is operably linked to a CMV promoter; a second nucleotide sequence, wherein the second nucleotide sequence encodes a first SARS-CoV-2 protein and is operably linked to promoter 1; and a third nucleotide sequence, wherein the third nucleotide sequence encodes a second SARS-CoV-2 protein and is operably linked to promoter 2. In some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes a first SARS-CoV-2 protein and is operably linked to promoter 1; and a third nucleotide sequence positioned 3′ to the second nucleotide sequence, wherein the third nucleotide sequence encodes a second SARS-CoV-2 protein and is operably linked to promoter 2. In some aspects, the first, second, and third nucleotide sequences of the polynucleotide are configured as shown in FIG. 4.

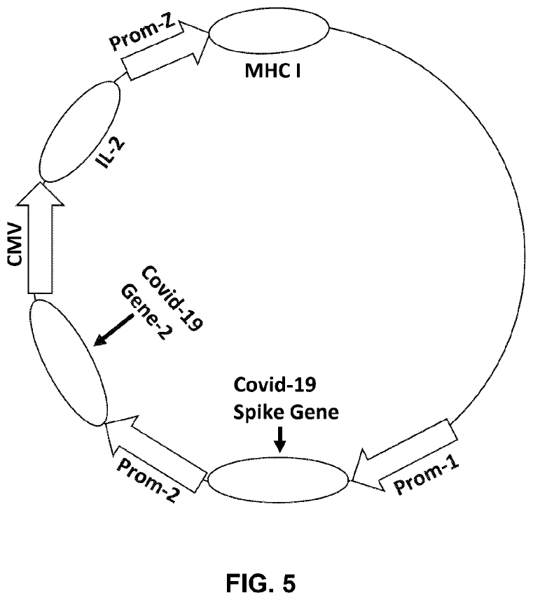

In some aspects, the compositions (e.g., pharmaceutical compositions or vaccines) of the disclosure comprise a delivery component and a polynucleotide comprising: a first nucleotide sequence, wherein the first nucleotide sequence encodes IL-2 and is operably linked to a CMV promoter; a second nucleotide sequence, wherein the second nucleotide sequence encodes MHC I and is operably linked to promoter Z; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes MHC I and is operably linked to promoter Z; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 5.

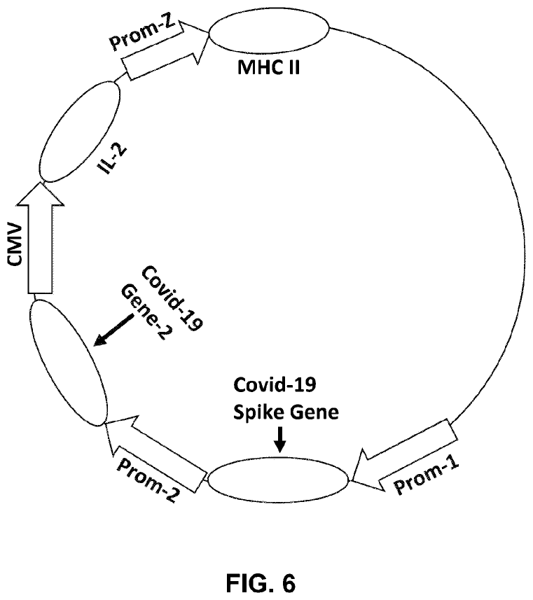

In some aspects, the compositions (e.g., pharmaceutical compositions or vaccines) of the disclosure comprise a delivery component and a polynucleotide comprising: a first nucleotide sequence, wherein the first nucleotide sequence encodes IL-2 and is operably linked to a CMV promoter; a second nucleotide sequence, wherein the second nucleotide sequence encodes MHC II and is operably linked to promoter Z; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes MHC II and is operably linked to promoter Z; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 6.

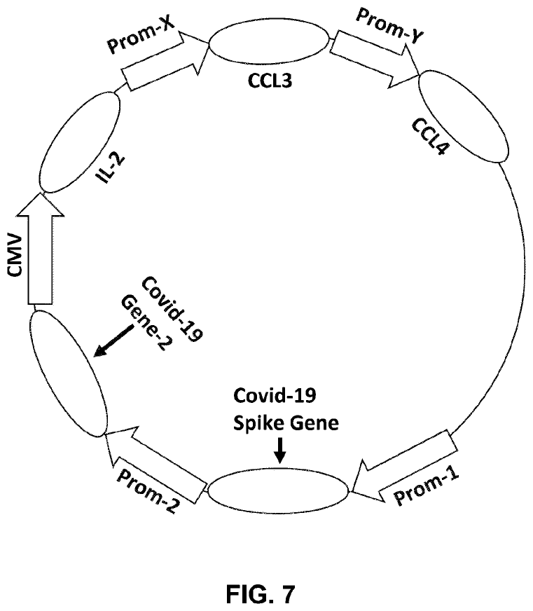

In some aspects, the compositions (e.g., pharmaceutical compositions or vaccines) of the disclosure comprise a delivery component and a polynucleotide comprising: a first nucleotide sequence, wherein the first nucleotide sequence encodes IL-2 and is operably linked to a CMV promoter; a second nucleotide sequence, wherein the second nucleotide sequence encodes CCL3 and is operably linked to promoter X; a third nucleotide sequence, wherein the third nucleotide sequence encodes CCL4 and is operably linked to promoter Y; a fourth nucleotide sequence, wherein the fourth nucleotide sequence encodes a first SARS-CoV-2 protein and is operably linked to promoter 1, and a fifth nucleotide sequence, wherein the fifth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes CCL3 and is operably linked to promoter X; a third nucleotide sequence positioned 3′ to the second nucleotide sequence, wherein the third nucleotide sequence encodes CCL4 and is operably linked to promoter Y; a fourth nucleotide sequence positioned 3′ to the third nucleotide sequence, wherein the fourth nucleotide sequence encodes a first SARS-CoV-2 protein and is operably linked to promoter 1, and a fifth nucleotide sequence positioned 3′ to the fourth nucleotide sequence, wherein the fifth nucleotide sequence encodes a second SARS-CoV-2 protein and is operably linked to promoter 2. In some aspects, the first, second, third, fourth, and fifth nucleotide sequences of the polynucleotide are configured as shown in FIG. 7.

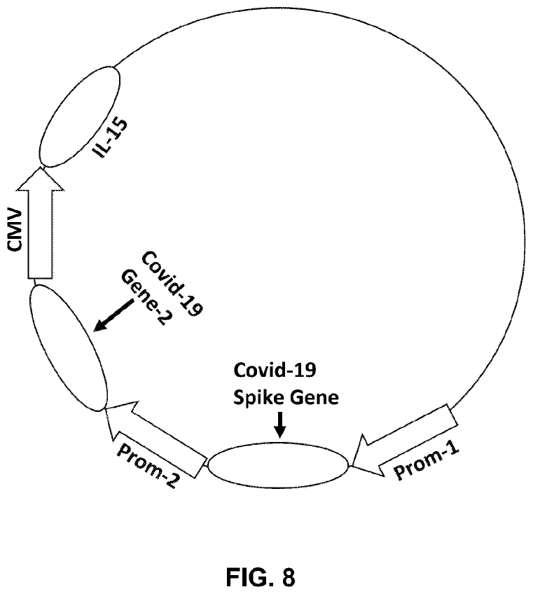

In some aspects, the compositions (e.g., pharmaceutical compositions or vaccines) of the disclosure comprise a delivery component and a polynucleotide comprising: a first nucleotide sequence, wherein the first nucleotide sequence encodes IL-15 and is operably linked to a CMV promoter; a second nucleotide sequence, wherein the second nucleotide sequence encodes a first SARS-CoV-2 protein and is operably linked to promoter 1; and a third nucleotide sequence, wherein the third nucleotide sequence encodes a second SARS-CoV-2 protein and is operably linked to promoter 2. In some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes a first SARS-CoV-2 protein and is operably linked to promoter 1; and a third nucleotide sequence positioned 3′ to the second nucleotide sequence, wherein the third nucleotide sequence encodes a second SARS-CoV-2 protein and is operably linked to promoter 2. In some aspects, the first, second, and third nucleotide sequences of the polynucleotide are configured as shown in FIG. 8.

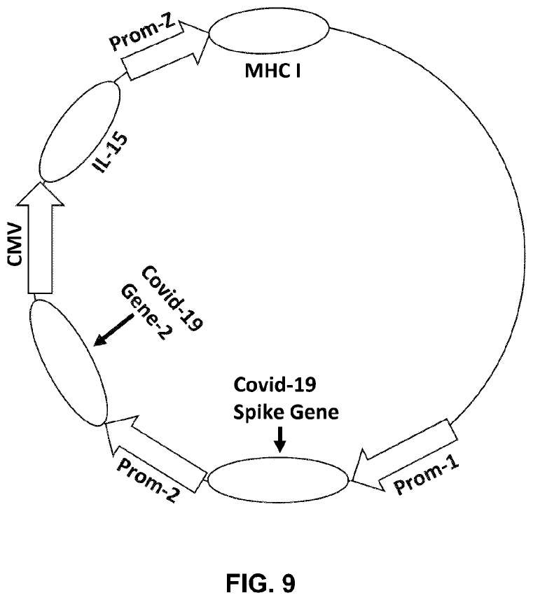

In some aspects, the compositions (e.g., pharmaceutical compositions or vaccines) of the disclosure comprise a delivery component and a polynucleotide comprising: a first nucleotide sequence, wherein the first nucleotide sequence encodes IL-15 and is operably linked to a CMV promoter; a second nucleotide sequence, wherein the second nucleotide sequence encodes MHC I and is operably linked to promoter Z; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes MHC I and is operably linked to promoter Z; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 9.

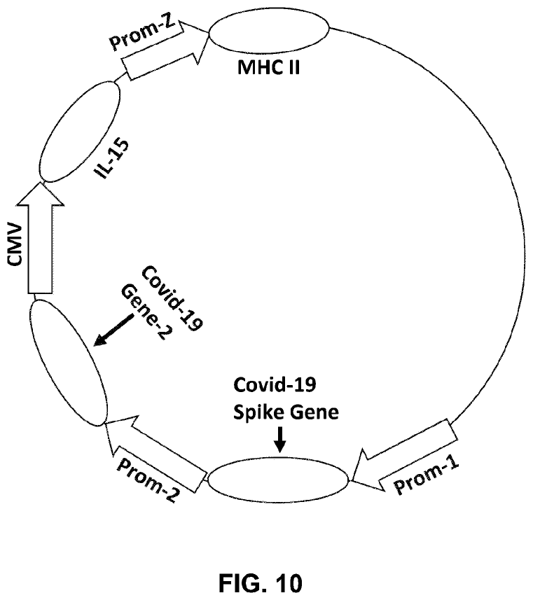

In some aspects, the compositions (e.g., pharmaceutical compositions or vaccines) of the disclosure comprise a delivery component and a polynucleotide comprising: a first nucleotide sequence, wherein the first nucleotide sequence encodes IL-15 and is operably linked to a CMV promoter; a second nucleotide sequence, wherein the second nucleotide sequence encodes MHC II and is operably linked to promoter Z; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes MHC II and is operably linked to promoter Z; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 10.

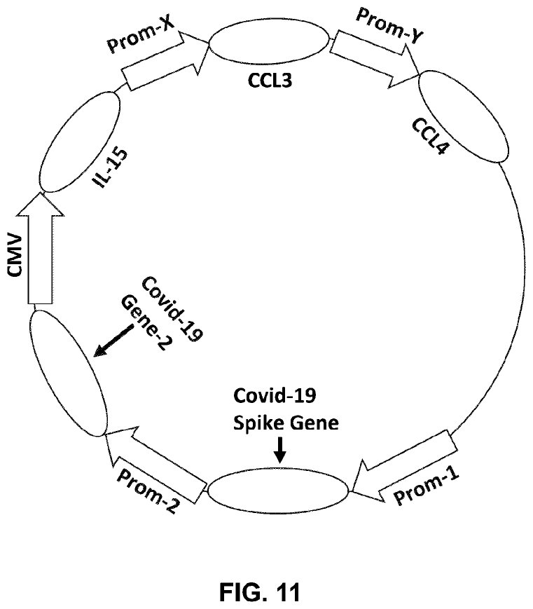

In some aspects, the compositions (e.g., pharmaceutical compositions or vaccines) of the disclosure comprise a delivery component and a polynucleotide comprising: a first nucleotide sequence, wherein the first nucleotide sequence encodes IL-15 and is operably linked to a CMV promoter; a second nucleotide sequence, wherein the second nucleotide sequence encodes CCL3 and is operably linked to promoter X; a third nucleotide sequence, wherein the third nucleotide sequence encodes CCL4 and is operably linked to promoter Y; a fourth nucleotide sequence, wherein the fourth nucleotide sequence encodes a first SARS-CoV-2 protein and is operably linked to promoter 1, and a fifth nucleotide sequence, wherein the fifth nucleotide sequence encodes a second SARS-CoV-2 protein and is operably linked to promoter 2. In some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes CCL3 and is operably linked to promoter X; a third nucleotide sequence positioned 3′ to the second nucleotide sequence, wherein the third nucleotide sequence encodes CCL4 and is operably linked to promoter Y; a fourth nucleotide sequence positioned 3′ to the third nucleotide sequence, wherein the fourth nucleotide sequence encodes a first SARS-CoV-2 protein and is operably linked to promoter 1, and a fifth nucleotide sequence positioned 3′ to the fourth nucleotide sequence, wherein the fifth nucleotide sequence encodes a second SARS-CoV-2 protein and is operably linked to promoter 2. In some aspects, the first, second, third, fourth, and fifth nucleotide sequences of the polynucleotide are configured as shown in FIG. 11.

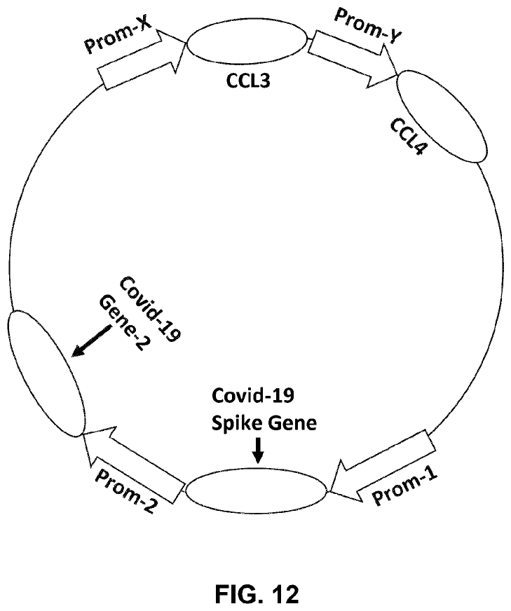

In some aspects, the compositions (e.g., pharmaceutical compositions or vaccines) of the disclosure comprise a delivery component and a polynucleotide comprising: a first nucleotide sequence, wherein the first nucleotide sequence encodes CCL3 and is operably linked to promoter X; a second nucleotide sequence, wherein the second nucleotide sequence encodes CCL4 and is operably linked to promoter Y; a third nucleotide sequence, wherein the third nucleotide sequence encodes a first SARS-CoV-2 protein and is operably linked to promoter 1; and a fourth nucleotide sequence, wherein the fourth nucleotide sequence encodes a second SARS-CoV-2 protein and is operably linked to promoter 2. In some aspects, the polynucleotide comprises: a 5′ first nucleotide sequence, wherein the first nucleotide sequence encodes CCL3 and is operably linked to promoter X; a second nucleotide sequence positioned 3′ to the first nucleotide sequence, wherein the second nucleotide sequence encodes CCL4 and is operably linked to promoter Y; a third nucleotide sequence positioned 3′ to the second nucleotide sequence, wherein the third nucleotide sequence encodes a first SARS-CoV-2 protein and is operably linked to promoter 1; and a fourth nucleotide sequence positioned 3′ to the third nucleotide sequence, wherein the fourth nucleotide sequence encodes a second SARS-CoV-2 protein and is operably linked to promoter 2. In some aspects, the first, second, third, and fourth nucleotide sequences of the polynucleotide are configured as shown in FIG. 12.

In some aspects, the compositions (e.g., pharmaceutical compositions or vaccines) can comprise a delivery component and a polynucleotides configured as shown in the vector constructs illustrated in any of FIGS. 1-12, which can modified to replace the “Covid-19 Spike Gene” (a first nucleotide sequence encoding a SARS-CoV-2 protein) and the “Covid-19 Gene-2” (a second nucleotide sequence encoding a SARS-CoV-2 protein) with nucleotide sequences encoding any combinations of pathogen antigen or antigenic fragment thereof disclosed herein. In some aspect, the nucleotide sequences encode antigens to a virus, a bacteria or a parasite. In some aspects, the nucleotide sequences encode one or more antigens comprise one or more viral antigens, one or more bacterial antigens, or one or more parasite antigens.

In some aspects, the compositions (e.g., pharmaceutical compositions or vaccines) of the disclosure comprise a delivery component and a polynucleotide comprising: a first nucleotide sequence, wherein the first nucleotide sequence encodes a first pathogen protein (e.g., a S1 subunit of the SARS-CoV-2 S protein) and is operably linked to a first promoter (e.g., a hEF1-HTLV promoter); a second nucleotide sequence, wherein the second nucleotide sequence encodes IL-12 p35 and is operably linked to a second promoter (e.g., a CMV promoter); and a third nucleotide sequence, wherein the third nucleotide sequence encodes IL-12 p40 and is operably linked to a second promoter (e.g., a CMV promoter). In some aspects, the first, second, and third nucleotide sequences of the polynucleotide are configured as shown in FIG. 14C (pVac 2).

In some aspects, the compositions (e.g., pharmaceutical compositions or vaccines) of the disclosure comprise a delivery component and a polynucleotide comprising: a first nucleotide sequence, wherein the first nucleotide sequence encodes a first pathogen protein (e.g., a S1 subunit of the SARS-CoV-2 S protein) and is operably linked to a first promoter (e.g., a hEF1-HTLV promoter); a second nucleotide sequence, wherein the second nucleotide sequence encodes a second pathogen protein (e.g., a SARS-CoV-2 M protein) and is operably linked to the first promoter through an IRES sequence; a third nucleotide sequence, wherein the third nucleotide sequence encodes IL-12 p35 and is operably linked to a second promoter (e.g., a CMV promoter); and a fourth nucleotide sequence, wherein the fourth nucleotide sequence encodes IL-12 p340 and is operably linked to a second promoter (e.g., a CMV promoter). In some aspects, the first, second, third, and fourth nucleotide sequences of the polynucleotide are configured as shown in FIG. 14D (pVac 3).

In some aspects, the compositions (e.g., pharmaceutical compositions or vaccines) of the disclosure comprise a delivery component and a polynucleotide comprising: a first nucleotide sequence, wherein the first nucleotide sequence encodes a first pathogen protein (e.g., a SARS-CoV-2 full-length D614G S protein) and is operably linked to a first promoter (e.g., an EF-1α promoter); a second nucleotide sequence, wherein the second nucleotide sequence encodes IL-12 p35 and is operably linked to a second promoter (e.g., a CMV promoter); and a third nucleotide sequence, wherein the third nucleotide sequence encodes IL-12 p40 and is operably linked to a second promoter (e.g., a CMV promoter). In some aspects, the first, second, and third nucleotide sequences of the polynucleotide are configured as shown in FIG. 14F (pVac 5).

In some aspects, the compositions (e.g., pharmaceutical compositions or vaccines) of the disclosure comprise a delivery component and a polynucleotide comprising: a first nucleotide sequence, wherein the first nucleotide sequence encodes a first pathogen protein (e.g., a full-length SARS-CoV-2 D614G S protein) and is operably linked to a first promoter (e.g., an EF-1α promoter); a second nucleotide sequence, wherein the second nucleotide sequence encodes a second pathogen protein (e.g., a SARS-CoV-2 M protein) and is operably linked to the first promoter through an IRES sequence; a third nucleotide sequence, wherein the third nucleotide sequence encodes IL-12 p35 and is operably linked to a second promoter (e.g., a CMV promoter); and a fourth nucleotide sequence, wherein the fourth nucleotide sequence encodes IL-12 p340 and is operably linked to a second promoter (e.g., a CMV promoter). In some aspects, the first, second, third, and fourth nucleotide sequences of the polynucleotide are configured as shown in FIG. 14G (pVac 6).

In some aspects, the compositions (e.g., pharmaceutical compositions or vaccines) of the disclosure comprise a delivery component and a polynucleotide comprising a first nucleotide sequence, wherein the first nucleotide sequence encodes a first pathogen protein (e.g., a S1 subunit of the SARS-CoV-2 S protein or a SARS-CoV-2 full-length D614G S protein) and is operably linked to a first promoter (e.g., an EF-1α promoter). In some aspects, the first nucleotide sequence of the polynucleotide is configured as shown in FIGS. 14B (pVac 1) or 14E (pVac 4).

In some aspects, the compositions (e.g., pharmaceutical compositions or vaccines) of the disclosure comprise a delivery component and a polynucleotide comprising a first nucleotide sequence, wherein the first nucleotide sequence encodes a first pathogen protein (e.g., a SARS-CoV-2 full-length D614G S protein) and is operably linked to a first promoter (e.g., an EF-1α promoter); and a second nucleotide sequence, wherein the second nucleotide sequence encodes a second pathogen (e.g., a SARS-CoV-2 M protein) and is operably linked to a second promoter (e.g., a CMV promoter). In some aspects, the first nucleotide sequence of the polynucleotide is configured as shown in FIG. 14H (pVac 7).

In some aspects, the compositions (e.g., pharmaceutical compositions or vaccines) of the disclosure comprise a polynucleotide comprising a nucleotide sequence, wherein the nucleotide sequence encodes a pathogen protein (e.g., any pathogen protein as disclosed herein) and is operably linked to a promoter (e.g., any promoter as disclosed herein) and a polyadenylation signal (e.g., any polyadenylation signal as disclosed herein). In some aspects, the polynucleotide comprises a nucleic acid sequence encoding one or more immune modifier proteins. In some aspects, the polynucleotide further comprises a intron (e.g., any intron as disclosed herein), and/or a 3′ UTR (e.g., any 3′ UTR as disclosed herein), and/or a leader sequence (e.g., any leader sequence as disclosed herein). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a nucleotide sequence, wherein the nucleotide sequence encodes a pathogen protein (e.g., any pathogen protein as disclosed herein) and is operably linked to a promoter (e.g., any promoter as disclosed herein) with an enhancer (e.g., any enhancer as disclosed herein) and a polyadenylation signal (e.g., any polyadenylation signal as disclosed herein). In some aspects, the polynucleotide comprises a nucleic acid sequence encoding one or more immune modifier proteins. In some aspects, the polynucleotide further comprises a intron (e.g., any intron as disclosed herein), and/or a 3′ UTR (e.g., any 3′ UTR as disclosed herein), and/or a leader sequence (e.g., any leader sequence as disclosed herein). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a first nucleotide sequence, wherein the first nucleotide sequence encodes a first pathogen protein (e.g., any pathogen protein as disclosed herein) and is operably linked to a first promoter (e.g., any promoter as disclosed herein) and a polyadenylation signal (e.g., any polyadenylation signal as disclosed herein); and a second nucleotide sequence, wherein the second nucleotide sequence encodes a second pathogen protein (e.g., any pathogen protein as disclosed herein) and is operably linked to a second promoter (e.g., any promoter as disclosed herein) and a polyadenylation signal (e.g., any polyadenylation signal as disclosed herein). In some aspects, the polynucleotide comprises a nucleic acid sequence encoding one or more immune modifier proteins. In some aspects, the polynucleotide further comprises a intron (e.g., any intron as disclosed herein), and/or a 3′ UTR (e.g., any 3′ UTR as disclosed herein), and/or a leader sequence (e.g., any leader sequence as disclosed herein). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a first nucleotide sequence, wherein the first nucleotide sequence encodes a first pathogen protein (e.g., any pathogen protein as disclosed herein) and is operably linked to a first promoter (e.g., any promoter as disclosed herein) with a first enhancer (e.g., any enhancer as disclosed herein) and a polyadenylation signal (e.g., any polyadenylation signal as disclosed herein); and a second nucleotide sequence, wherein the second nucleotide sequence encodes a second pathogen protein (e.g., any pathogen protein as disclosed herein) and is operably linked to a second promoter (e.g., any promoter as disclosed herein) with a second enhancer (e.g., any enhancer as disclosed herein) and a polyadenylation signal (e.g., any polyadenylation signal as disclosed herein). In some aspects, the polynucleotide comprises a nucleic acid sequence encoding one or more immune modifier proteins. In some aspects, the polynucleotide further comprises a intron (e.g., any intron as disclosed herein), and/or a 3′ UTR (e.g., any 3′ UTR as disclosed herein), and/or a leader sequence (e.g., any leader sequence as disclosed herein). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a first nucleotide sequence, wherein the first nucleotide sequence encodes a first pathogen protein (e.g., any pathogen protein as disclosed herein) and is operably linked to a first promoter (e.g., any promoter as disclosed herein) and a polyadenylation signal (e.g., any polyadenylation signal as disclosed herein); a second nucleotide sequence, wherein the second nucleotide sequence encodes a second pathogen protein (e.g., any pathogen protein as disclosed herein) and is operably linked to a second promoter (e.g., any promoter as disclosed herein) and a polyadenylation signal (e.g., any polyadenylation signal as disclosed herein); and a third nucleotide sequence, wherein the third nucleotide sequence encodes a third pathogen protein (e.g., any pathogen protein as disclosed herein) and is operably linked to a third promoter (e.g., any promoter as disclosed herein) and a polyadenylation signal (e.g., any polyadenylation signal as disclosed herein). In some aspects, the polynucleotide comprises a nucleic acid sequence encoding one or more immune modifier proteins. In some aspects, the polynucleotide further comprises a intron (e.g., any intron as disclosed herein), and/or a 3′ UTR (e.g., any 3′ UTR as disclosed herein), and/or a leader sequence (e.g., any leader sequence as disclosed herein). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a first nucleotide sequence, wherein the first nucleotide sequence encodes a first pathogen protein (e.g., any pathogen protein as disclosed herein) and is operably linked to a first promoter (e.g., any promoter as disclosed herein) with a first enhancer (e.g., any enhancer as disclosed herein) and a polyadenylation signal (e.g., any polyadenylation signal as disclosed herein); a second nucleotide sequence, wherein the second nucleotide sequence encodes a second pathogen protein (e.g., any pathogen protein as disclosed herein) and is operably linked to a second promoter (e.g., any promoter as disclosed herein) with a second enhancer (e.g., any enhancer as disclosed herein) and a polyadenylation signal (e.g., any polyadenylation signal as disclosed herein); and a third nucleotide sequence, wherein the third nucleotide sequence encodes a second pathogen protein (e.g., any pathogen protein as disclosed herein) and is operably linked to a third promoter (e.g., any promoter as disclosed herein) with a third enhancer (e.g., any enhancer as disclosed herein) and a polyadenylation signal (e.g., any polyadenylation signal as disclosed herein). In some aspects, the polynucleotide comprises a nucleic acid sequence encoding one or more immune modifier proteins. In some aspects, the polynucleotide further comprises a intron (e.g., any intron as disclosed herein), and/or a 3′ UTR (e.g., any 3′ UTR as disclosed herein), and/or a leader sequence (e.g., any leader sequence as disclosed herein). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a first nucleotide sequence, wherein the first nucleotide sequence encodes a first pathogen protein (e.g., a SARS-CoV-2 full-length D614G spike protein with 2P modification) and is operably linked to a first promoter (e.g., a CMV promoter) and a polyadenylation signal (e.g., a SV40 polyA); and a second nucleotide sequence, wherein the second nucleotide sequence encodes a second pathogen (e.g., a SARS-CoV-2 Delta variant spike protein with 2P modification) which is operably linked to a second promoter (e.g., a CMV promoter) and a polyadenylation signal (e.g., a SV40 polyA). In some aspects, the nucleotide sequences of the polynucleotide is configured as shown in FIG. 14AR (pVac43). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a nucleotide sequence, wherein the nucleotide sequence encodes a pathogen protein (e.g., a SARS-CoV-2 BA.2.75.2 variant spike protein) and is operably linked to a promoter (e.g., a CMV promoter) and a polyadenylation signal (e.g., a bGH polyA). In some aspects, the nucleotide sequences of the polynucleotide is configured as shown in FIG. 14AZ (pVac51). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a nucleotide sequence, wherein the nucleotide sequence encodes a pathogen protein (e.g., SARS-CoV-2 BA.2.75.2 variant spike protein) with a leader sequence (e.g., an IgE leader sequence) and is operably linked to a promoter (e.g., a CMV promoter) and a polyadenylation signal (e.g., a bGH polyA). In some aspects, the nucleotide sequences of the polynucleotide is configured as shown in FIG. 14AAA (pVac52). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a nucleotide sequence, wherein the nucleotide sequence encodes a pathogen protein (e.g., a SARS-CoV-2 Delta variant spike protein with 2P modification) and is operably linked to a promoter (e.g., a CMV promoter) with an enhancer (e.g., a SV40 enhancer) and a polyadenylation signal (e.g., a bGH polyA). In some aspects, the nucleotide sequences of the polynucleotide is configured as shown in FIG. 14AAB (pVac53). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a first nucleotide sequence, wherein the first nucleotide sequence encodes a first pathogen protein (e.g., a SARS-CoV-2 Delta variant spike protein with 2P modification) and is operably linked to a first promoter (e.g., a CMV promoter) with a first enhancer (e.g., a SV40 enhancer) and a polyadenylation signal (e.g., a bGH polyA); and a second nucleotide sequence, wherein the second nucleotide sequence encodes a second pathogen protein (e.g., a SARS-CoV-2 full-length D614G spike protein with 2P modification) and is operably linked to a second promoter (e.g,. a CMV promoter) with a second enhancer (e.g., a SV40 enhancer) and a polyadenylation signal (e.g., a bGH polyA). In some aspects, the nucleotide sequences of the polynucleotide is configured as shown in FIG. 14AAC (pVac54). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a nucleotide sequence, wherein the nucleotide sequence encodes a pathogen protein (e.g., a SARS-CoV-2 22E variant spike protein with 2P modification) and is operably linked to a promoter (e.g,. a CMV promoter) with an enhancer (e.g., a SV40 enhancer) and a polyadenylation signal (e.g., a bGH polyA). In some aspects, the nucleotide sequences of the polynucleotide is configured as shown in FIG. 14AAD (pVac55). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a nucleotide sequence, wherein the nucleotide sequence encodes a pathogen protein (e.g., a SARS-CoV-2 22E variant spike protein with 2P modification and furin cleavage mutation) and is operably linked to a promoter (e.g., a CMV promoter) with an enhancer (e.g., a SV40 enhancer) and a polyadenylation signal (e.g., a bGH polyA). In some aspects, the nucleotide sequences of the polynucleotide is configured as shown in FIG. 14AAE (pVac56). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a nucleotide sequence, wherein the nucleotide sequence encodes a pathogen protein (e.g., a SARS-CoV-2 Beta variant spike protein with 2P modification and furin cleavage mutation) and is operably linked to a promoter (e.g., a CMV promoter) with an enhancer (e.g., a SV40 enhancer) and a polyadenylation signal (e.g., a bGH polyA). In some aspects, the nucleotide sequences of the polynucleotide is configured as shown in FIG. 14AAF (pVac57). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a nucleotide sequence, wherein the nucleotide sequence encodes a pathogen protein (e.g.. a SARS-CoV-2 nucleocapsid protein) and is operably linked to a promoter (e.g., a CMV promoter) with an enhancer (e.g., a SV40 enhancer). In some aspects, the nucleotide sequences of the polynucleotide is configured as shown in FIG. 14AAG (pVac58). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a first nucleotide sequence, wherein the first nucleotide sequence encodes a first pathogen protein (e.g., a SARS-CoV-2 Beta variant with 2P modification and furin cleavage mutation) and is operably linked to a first promoter (e.g., a CMV promoter) with an enhancer (e.g., a SV40 enhancer) and a polyadenylation signal (e.g., a bGH polyA); and a second nucleotide sequence, wherein the second nucleotide sequence encodes a second pathogen protein (e.g., a SARS-CoV-2 22E variant spike protein with 2P modification and furin cleavage mutation) and is operably linked to a second promoter (e.g., a CMV promoter) with an enhancer (e.g., a SV40 enhancer) and a polyadenylation signal (e.g., a bGH polyA). In some aspects, the nucleotide sequences of the polynucleotide is configured as shown in FIG. 14AAH (pVac59). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a first nucleotide sequence, wherein the first nucleotide sequence encodes a first pathogen protein (e.g., a SARS-CoV-2 Beta variant spike protein with 2P modification and furin cleavage mutation) and is operably linked to a first promoter (e.g., a CMV promoter) with a first enhancer (e.g., a SV40 enhancer) and a polyadenylation signal (e.g., a bGH polyA); a second nucleotide sequence, wherein the second nucleotide sequence encodes a second pathogen protein (e.g., a SARS-CoV-2 22E variant spike protein with 2P modification and furin cleavage mutation) and is operably linked to a second promoter (e.g., a CMV promoter) with a second enhancer (e.g., a SV40 enhancer) and a polyadenylation signal (e.g., a bGH polyA); and a third nucleotide sequence, wherein the third nucleotide sequence encodes a third pathogen protein (e.g., a SARS-CoV-2 nucleocapsid protein) and is operably linked to a third promoter (e.g., a CMV promoter) with a third enhancer (e.g., a SV40 enhancer) and a polyadenylation signal (e.g, a bGH polyA). In some aspects, the nucleotide sequences of the polynucleotide is configured as shown in FIG. 14AAI (pVac60). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a first nucleotide sequence, wherein the first nucleotide sequence encodes a first pathogen protein (e.g., a SARS-CoV-2 omicron BA.5 spike protein) and is operably linked to a first promoter (e.g., a CMV promoter) with a first enhancer (e.g., a SV40 enhancer) and a polyadenylation signal (e.g., a bGH polyA); a second nucleotide sequence, wherein the second nucleotide sequence encodes a second pathogen protein (e.g., a SARS-CoV-2 full-length D614G spike protein with 2P modification) and is operably linked to a second promoter (e.g, a CMV promoter) with a second enhancer (e.g., a SV40 enhancer) and a polyadenylation signal (e.g., a bGH polyA); and a third nucleotide sequence, wherein the third nucleotide sequence encodes a third pathogen protein (e.g., a SARS-CoV-2 nucleocapsid protein) and is operably linked to a third promoter (e.g, a CMV promoter) with a third enhancer (e.g., a SV40 enhancer) and a polyadenylation signal (e.g., a bGH polyA). In some aspects, the nucleotide sequences of the polynucleotide is configured as shown in FIG. 14AAJ (pVac61). In some aspects, the composition further comprises (i) a delivery component (e.g., a poloxamer) and (ii) an adjuvant (e.g., an aluminum or aluminum salt).

In some aspects, the compositions (e.g., pharmaceutical compositions or vaccines) of the disclosure comprise a polynucleotide comprising a first nucleotide sequence, wherein the first nucleotide sequence encodes a first pathogen protein (e.g., a SARS-CoV-2 omicron variant BQ.1 nucleocapsid protein) and is operably linked to a first promoter (e.g., a CMV promoter) with a first enhancer (e.g., a SV40 enhancer) and a polyadenylation signal (e.g., a bGH polyA); and a second nucleotide sequence, wherein the second nucleotide sequence encodes a second pathogen protein (e.g., a SARS-CoV-2 omicron variant XBB1.5 spike protein) and is operably linked to a second promoter (e.g., a CMV promoter) and a second enhancer (e.g., a SV40 enhancer) and a polyadenylation signal (e.g, a bGH polyA). In some aspects, the nucleotide sequences of the polynucleotide is configured as shown in FIG. 14AAK (pVac62). In some aspects, the composition further comprises (i) a delivery component (e.g., a poloxamer) and (ii) an adjuvant (e.g., an aluminum or aluminum salt).

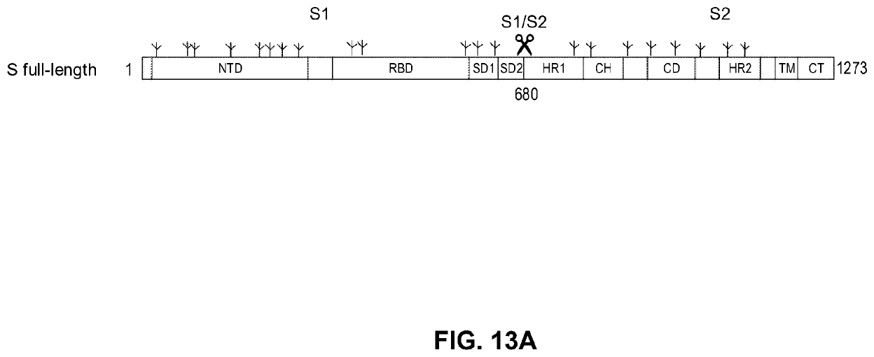

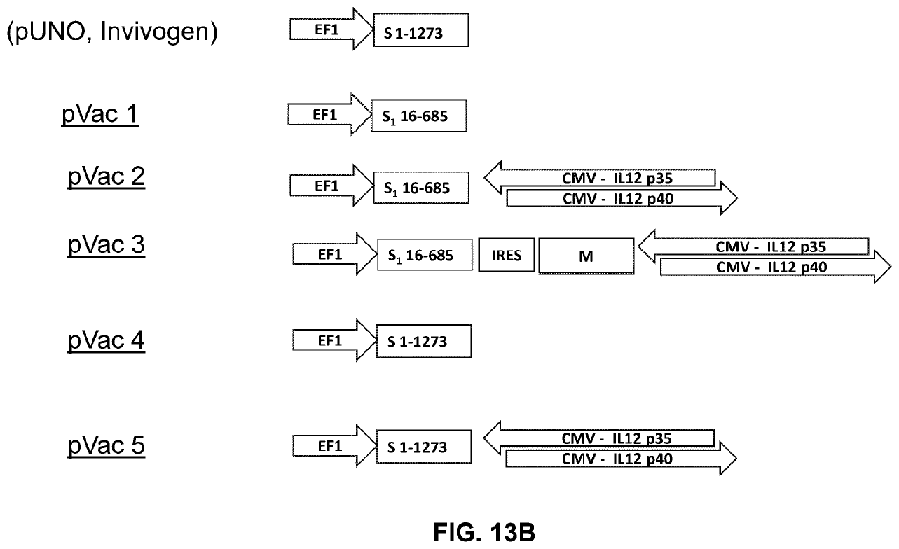

In some aspects, the composition (e.g., pharmaceutical compositions or vaccines) can comprise a delivery component, wherein the polynucleotide configured as shown in the vector constructs illustrated in any of FIGS. 13B, 14B-14H and 14J-14AAK (pVac 1-38, 40, and 42-62) can be modified to replace the S1 subunit of the SARS-CoV-2 S protein or the SARS-CoV-2 full-length D614G S protein (a first nucleotide sequence encoding a first pathogen protein) and/or the SARS-CoV-2 M protein (a second nucleotide sequence encoding a second pathogen protein) and/or the SARS-CoV-2 nucleocapsid (N) protein (a third nucleotide sequence encoding a third pathogen protein) with nucleotide sequences encoding any combinations of pathogen antigen or antigenic fragment thereof disclosed herein. In some aspect, the nucleotide sequences encode antigens to a virus, a bacteria or a parasite. In some aspects, the nucleotide sequences encode one or more antigens comprise one or more viral antigens, one or more bacterial antigens, or one or more parasite antigens.

In some aspects, the compositions (pharmaceutical compositions or vaccines) can comprise an adjuvant and a polynucleotide configured as shown in the vector constructs illustrated in any of FIGS. 1-14B-14H and 14J-14AAK and/or vector constructs that have been modified to replace the S1 subunit of the SARS-CoV-2 S protein or the SARS-CoV-2 full-length D614G S protein (a first nucleotide sequence encoding a first pathogen protein) and/or the SARS-CoV-2 M protein (a second nucleotide sequence encoding a second pathogen protein) and/or the SARS-CoV-2 nucleocapsid (N) protein (a third nucleotide sequence encoding a third pathogen protein) with nucleotide sequences encoding any combinations of pathogen antigen or antigenic fragment thereof disclosed herein.

In some aspects, the compositions (pharmaceutical compositions or vaccines) can comprise a polynucleotide encoding any combinations of pathogen antigen or antigenic fragment thereof disclosed herein and an adjuvant selected from potassium aluminum sulfate [KAl(SO₄)₂], aluminum hydroxide, crystalline aluminum oxyhydroxide, aluminum phosphate, aluminum hydroxyphosphate, amorphous aluminum hydroxyphosphate sulfate, aluminum chloride, aluminum silicate, and a composition comprising aluminum hydroxide and magnesium hydroxide.

In some aspects, the compositions (pharmaceutical compositions or vaccines) can comprise a polynucleotide encoding any combinations of pathogen antigen or antigenic fragment thereof disclosed herein and a STING agonist.

In some aspects, the compositions (pharmaceutical compositions or vaccines) can comprise a polynucleotide encoding any combinations of pathogen antigen or antigenic fragment thereof disclosed herein and the STING agonist cyclic GMP (cGMP).

In some aspects, the compositions (pharmaceutical compositions or vaccines) can comprise a polynucleotide encoding any combinations of pathogen antigen or antigenic fragment thereof disclosed herein and a STING agonist selected from ADU-S100, MK-1454, SB11285, BMS-986301, BI-STING (BI1387446), JNJ-67544412, 3′3′-cyclic AIMIP, and GSK532.

In some aspects, the compositions (pharmaceutical compositions or vaccines) can comprise a polynucleotide encoding any combinations of pathogen antigen or antigenic fragment thereof disclosed herein and a STING agonist selected from DMAXAA, ALG-031048, E7766, JNJ-′6196, MK-21118, MSA-1, MSA-2, SNX281, SR-717, TAK676, TTI-10001, a Ryvu's agonist, GF3-002, a Selvita agonist, CDR5500, CS-1010, CS-1018, CS-1020, and [2-ex] MSA-1.

In some aspects, the compositions (pharmaceutical compositions or vaccines) can comprise a polynucleotide encoding any combinations of pathogen antigen or antigenic fragment thereof disclosed herein and a STING agonist selected from a PC7A nanoparticle, a cGMP-nanoparticle, and a ONM-500 nanoparticle.

In some aspects, the compositions (pharmaceutical compositions or vaccines) can comprise a polynucleotide encoding any combinations of pathogen antigen or antigenic fragment thereof disclosed herein and a STING agonist that is an antibody-drug conjugate. In some aspects, the antibody drug conjugate is XMT-2056.

In some aspects, the compositions (pharmaceutical compositions or vaccines) can comprise a polynucleotide encoding any combinations of pathogen antigen or antigenic fragment thereof disclosed herein and a STING agonist that is an ENPP1 inhibitor selected from MV-626, SR-8314, SR-8291, and SR8541A.

In some aspects, the compositions (pharmaceutical compositions or vaccines) can comprise a polynucleotide encoding any combinations of pathogen antigen or antigenic fragment thereof disclosed herein and a STING agonist that is a bacterial vector. In some aspects, the bacterial vector is a nonpathogenic E. coli nissle expressing cyclic-di-AMP-producing enzymes. In some aspects, the bacterial vector is SYNB1981. In some aspects, the bacterial vector is an attenuated Salmonella Typhimurium strain engineered to carry an inhibitory TREX-1 micro RNA.

In some aspects, the compositions (pharmaceutical compositions or vaccines) can comprise a polynucleotide encoding any combinations of pathogen antigen or antigenic fragment thereof disclosed herein, an adjuvant and a STING agonist.

In some aspects, the compositions (pharmaceutical compositions or vaccines) can comprise a polynucleotide encoding any combinations of pathogen antigen or antigenic fragment thereof disclosed herein, an adjuvant selected from potassium aluminum sulfate [KAl(SO₄)₂], aluminum hydroxide, crystalline aluminum oxyhydroxide, aluminum phosphate, aluminum hydroxyphosphate, amorphous aluminum hydroxyphosphate sulfate, aluminum chloride, aluminum silicate, a composition comprising aluminum hydroxide and magnesium hydroxide and combinations thereof, and a STING agonist selected from cyclic GMP (cGMP), cAMP, and cGAMP.

In some aspects, the compositions (pharmaceutical compositions or vaccines) can comprise a polynucleotide encoding any combinations of pathogen antigen or antigenic fragment thereof disclosed herein, an adjuvant selected from potassium aluminum sulfate [KAl(SO₄)₂], aluminum hydroxide, crystalline aluminum oxyhydroxide, aluminum phosphate, aluminum hydroxyphosphate, amorphous aluminum hydroxyphosphate sulfate, aluminum chloride, aluminum silicate, a composition comprising aluminum hydroxide and magnesium hydroxide and combinations thereof, and a STING agonist selected from cyclic GMP (cGMP), ADU-S100, MK-1454, SB11285, BMS-986301, BI-STING (BI1387446), JNJ-67544412, 3′3′-cyclic AIMP, GSK532, DMAXAA, ALG-031048, E7766, JNJ-′6196, MK-21118, MSA-1, MSA-2, SNX281, SR-717, TAK676, TTI-10001, a Ryvu's agonist, GF3-002, a Selvita agonist, CDR5500, CS-1010, CS-1018, CS-1020, [2-ex] MSA-1, a PC7A nanoparticle, a cGMP-nanoparticle, and a ONM-500 nanoparticle, the XMT-2056 antibody-drug conjugate, an ENPP1 inhibitor selected from MV-626, SR-8314, SR-8291, and SR8541A, a bacterial vector selected from SYNB1981 and an attenuated Salmonella Typhimurium strain engineered to carry an inhibitory TREX-1 micro RNA.

In some aspects, the composition comprises between 0.001 and 1 mg/ml STING agonist. In some aspects, the composition comprises between 0.005 and 0.8 mg/ml STING agonist. In some aspects, the composition comprises between 0.01 and 0.75 mg/ml STING agonist. In some aspects, the composition comprises between 0.05 and 0.5 mg/ml STING agonist. In some aspects, the composition comprises between 0.1 and 0.4 mg/ml STING agonist.

In some aspects, the compositions (e.g., pharmaceutical composition or vaccine) further comprise a delivery component (e.g., a cationic polymer, a poly-inosinic-polycytidylic acid, or a poloxamer). In some aspects, the delivery component further comprises benzalkonium chloride.

The polynucleotides and compositions of the present disclosure (e.g., pharmaceutical compositions, vaccines, vectors, and DNA plasmid vectors) can be formulated according to known methods for preparing pharmaceutically useful compositions.

Formulations are described in a number of sources which are well known and readily available to those skilled in the art. For example, Remington's Pharmaceutical Science (Martin E W [1995] Easton Pennsylvania, Mack Publishing Company, 19th Ed.) describes formulations, which can be used in connection with the subject disclosure. Formulations suitable for parenteral administration include, for example, aqueous sterile injection solutions, which can contain antioxidants, buffers, bacteriostats, and solutes which render the formulation isotonic with the blood of the intended recipient; and aqueous and nonaqueous sterile suspensions which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the formulations of the subject disclosure can include other agents conventional in the art having regard to the type of formulation in question.

The disclosure also provides lyophilized (or freeze-dried) compositions or vaccines that can be safely stored for periods of time and reconstituted prior to use. In some aspects, the composition, pharmaceutical composition, or vaccine of the disclosure is a lyophilized product, e.g., substantially free of aqueous components. In some aspects, the lyophilized composition or vaccine is reconstituted in a diluent, e.g., prior to administration. In some aspects, the lyophilized composition or vaccine is reconstituted in water.

Some aspects are directed to a lyophilized composition or vaccine comprising: (1) any polynucleotide or DNA plasmid vector disclosed herein; (2) a STING agonist; (3) an aluminum or aluminum salt based adjuvant; and (4) any delivery component disclosed herein, wherein the composition is substantially free of aqueous components. In some aspects, a composition or vaccine of the disclosure is lyophilized.

Some aspects are directed to a lyophilized composition or vaccine comprising: (1) any polynucleotide or DNA plasmid vector disclosed herein; (2) an aluminum or aluminum salt based adjuvant; and (3) any delivery component disclosed herein, wherein the composition is substantially free of aqueous components. In some aspects, a composition or vaccine of the disclosure is lyophilized.

In some aspects, the composition or vaccine is stable at 0° C. to 5° C. for at least about 1 month (or 30 days), 2 months, 3 months, 4 months, 5 months, 6 months, 7 months, 8 months, 9 months, 10 months, 11 months, or 12 months. In some aspects, the composition or vaccine is stable at 0° C. to 5° C. for at least 1 year, at least 2 years, at least 3 years, at least 4 years or at least 5 years. In some aspects, the composition or vaccine is stable at 25° C. for at least about 7 days, about 10 day, or about 14 days. In some aspects, the composition or vaccine is lyophilized and is stable at 0° C. to 5° C. for at least about 1 month (or 30 days), 2 months, 3 months, 4 months, 5 months, 6 months, 7 months, 8 months, 9 months, 10 months, 11 months, or 12 months. In some aspects, the lyophilized composition or vaccine is stable at 0° C. to 5° C. for at least 1 year, at least 2 years, at least 3 years, at least 4 years or at least 5 years. In some aspects, the lyophilized composition or vaccine is stable at 25° C. for at least about 7 days, about 10 day, or about 14 days.

In some aspects, the lyophilized composition or vaccine is reconstituted into a reconstituted composition or vaccine formulation for administration. In some aspects, the reconstituted composition or vaccine is stable at 0° C. to 5° C. for at least about 1 month, about 2 months, about 3 months, about 4 months, about 5 months, or about 6 months after reconstitution of the lyophilized composition or vaccine with a diluent (e.g., water). In some aspects, the reconstituted composition or vaccine is stable at 25° C. for at least about 2 days, about 3 days, about 4 days, about 5 days, about 6 days, or about 7 days after reconstitution of the lyophilized composition with a diluent. In some aspects, the diluent is water.

The compositions of the subject disclosure can further comprise other components such as a pharmaceutically acceptable carrier and/or an immune modifier protein. The immune modifier protein can be expressed in alternative polynucleotides, plasmids, or vectors or are delivered as proteins in combination with the compositions of the subject disclosure. The immune modifier protein can be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHIC, CD80, CD86 including IL-15 having the signal sequence deleted and optionally including the signal peptide from IgE. The immune modifier protein can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof. Other genes that can be useful immune modifier proteins include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, INK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2, functional fragments thereof, and combinations thereof.

In some aspects, compositions of the disclosure can be formulated according to the mode of administration to be used. For example, an injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions.

### IV. Polynucleotides

In some aspects, the polynucleotides of the disclosure can include DNA or mRNA sequences (e.g., expression vector, multicistronic DNA or multicistronic mRNA) for use in the compositions (e.g., pharmaceutical compositions and vaccines) disclosed herein. In some aspects, the present disclosure is directed to a polynucleotide (e.g., expression vector) comprising a nucleic acid sequence encoding a single pathogen protein or antigenic fragments thereof (e.g., a SARS CoV-2 antigen or monkeypox antigen). In some aspects, the vector further comprises a nucleic acid sequence encoding one or more immune modifier proteins. In some aspects, the polynucleotide (e.g., expression vector) can comprise a nucleic acid sequence encoding one or more viral antigens or antigenic fragments thereof (e.g., a SARS CoV-2 antigen or monkeypox antigen). In some aspects, the vector further comprises a nucleic acid sequence encoding one or more immune modifier proteins. In some aspects, the polynucleotide comprises nucleic acids encoding at least two pathogen proteins or antigenic fragments thereof. In some aspects, the polynucleotide comprises one, two, three, four, five six, seven, or eight nucleic acids encoding different pathogen proteins or antigenic fragments thereof. In some aspects, the vector comprises a nucleic acid sequence encoding a SARS CoV-2 antigen and, optionally, a second viral antigen. In some aspects, the vector comprises a nucleic acid sequence encoding a SARS-CoV-2 antigen and a second SARS-CoV-2 antigen. In some aspects, the nucleic acid sequence encodes a SARS-CoV-2 antigen and a second SARS-CoV-2 antigen from a different SARS-CoV-2 strain. In some aspects, the vector comprises a nucleic acid sequence encoding a SARS-CoV-2 antigen, a second SARS-CoV-2 antigen, and a third SARS-CoV-2 antigen. In some aspects, the nucleic acid sequence encodes different variants of the same SARS-CoV-2 antigen, wherein the different variants of the same SARS-CoV-2 antigen are derived from different strains of SARS CoV-2. In some aspects, the nucleic acid sequence encodes different variants of a SARS-CoV-2 S protein antigen, wherein the different variants of the SARS-CoV-2 S protein antigens are derived from different strains of SARS CoV-2.

In some aspects, the vector comprises a nucleic acid sequence encoding a monkeypox antigen and, optionally, a second viral antigen. In some aspects, the vector comprises a nucleic acid sequence encoding a monkeypox antigen and a second monkeypox antigen. In some aspects, the nucleic acid sequence encodes a monkeypox antigen and a second monkeypox antigen from a different monkeypox strain. In some aspects, the nucleic acid sequence encodes different variants of the same monkeypox antigen, wherein the different variants of the same monkeypox antigen are derived from different strains of monkeypox. In some aspects, the nucleic acid sequence encodes different variants of a monkeypox A35R protein antigen, wherein the different variants of the monkeypox A35R protein antigens are derived from different strains of monkeypox. In some aspects, the nucleic acid sequence encodes different variants of a monkeypox H3L protein antigen, wherein the different variants of the monkeypox H3L protein antigens are derived from different strains of monkeypox. In some aspects, the nucleic acid sequence encodes different variants of a monkeypox L1R protein antigen, wherein the different variants of the monkeypox L1R protein antigens are derived from different strains of monkeypox.

In some aspects, the present disclosure is directed to a polynucleotide (e.g., expression vector) comprising: (a) an antigen nucleic acid, which encodes a pathogen protein antigen (e.g., a viral antigen, a bacterial antigen, or a parasite antigen) or an antigenic fragment thereof, and (b) a nucleic acid encoding an immune modifier protein. In some aspects, the present disclosure is directed to a polynucleotide (e.g., expression vector) comprising: (a) an antigen nucleic acid (e.g., first antigen nucleic acid) which encodes a pathogen protein antigen (e.g., a viral antigen, a bacterial antigen, or a parasite antigen) or an antigenic fragment thereof; and (b) a nucleic acid encoding an immune modifier protein. In some aspects, the antigen nucleic acid (e.g., first antigen nucleic acid) is operably linked to a first promoter. In some aspects, the polynucleotide (e.g., expression vector) comprises two or more nucleic acids encoding an immune modifier protein. In some aspects, each of the nucleic acids encoding an immune modifier protein encodes a different immune modifier protein.

The polynucleotides (e.g., expression vector) disclosed herein can comprise: (b) or (c) at least one additional antigen nucleic acid (e.g., second antigen nucleic acid) which encodes a second pathogen protein antigen (e.g., a viral antigen, a bacterial antigen, or a parasite antigen) or an antigenic fragment thereof. In some aspects, the second pathogen protein or antigenic fragment thereof comprises one or more viral antigens, one or more bacterial antigens, or one or more parasite antigens. In some aspects, the first pathogen antigen and the second pathogen antigen are SARS-CoV-2 antigens from different strains of SARS-CoV-2. In some aspects, the first pathogen antigen and the second pathogen antigens are different variants of the same SARS-CoV-2 antigen, wherein the different variants of the same SARS-CoV-2 antigen are derived from different strains of SARS-CoV-2. In some aspects, the first pathogen antigen and the second pathogen antigen are different variants of a SARS-CoV-2 S protein antigen, wherein the different variants of the SARS-CoV-2 S protein antigen are derived from different strains of SARS CoV-2.

In some aspects, the present disclosure is directed to a polynucleotide (e.g., expression vector) comprising: (a) a first antigen nucleic acid, which encodes a first pathogen protein antigen (e.g., a viral antigen, a bacterial antigen, or a parasite antigen) or an antigenic fragment thereof; (b) a second antigen nucleic acid, which encodes a second pathogen protein antigen (e.g., a viral antigen, a bacterial antigen, or a parasite antigen) or an antigenic fragment thereof; and (c) a third antigen nucleic acid, which encodes a third pathogen protein antigen (e.g., a viral antigen, a bacterial antigen, or a parasite antigen) or an antigenic fragment thereof.

In some aspects, the first pathogen antigen, the second pathogen antigen, and the third pathogen antigen are SARS-CoV-2 antigens. In some aspects, the first pathogen antigen, the second pathogen antigen, and the third pathogen antigen are SARS-CoV-2 antigens from different strains of SARS-CoV-2. In some aspects, the first pathogen antigen and the second pathogen antigen are SARS-CoV-2 antigens from different strains of SARS-CoV-2. In some aspects, the first pathogen antigen and the second pathogen antigens are different variants of the same SARS-CoV-2 antigen, wherein the different variants of the same SARS-CoV-2 antigen are derived from different strains of SARS-CoV-2. In some aspects, the first pathogen antigen and the second pathogen antigen are different variants of a SARS-CoV-2 S protein antigen, wherein the different variants of the SARS-CoV-2 S protein antigen are derived from different strains of SARS CoV-2.

In some aspects, the first pathogen antigen and the second pathogen antigen are monkeypox antigens from different strains of monkeypox. In some aspects, the first pathogen antigen and the second pathogen antigens are different variants of the same monkeypox antigen, wherein the different variants of the same monkeypox antigen are derived from different strains of monkeypox. In some aspects, the first pathogen antigen and the second pathogen antigen are different variants of a monkeypox A35R protein antigen, wherein the different variants of the monkeypox A35R protein antigen are derived from different strains of monkeypox. In some aspects, the first pathogen antigen and the second pathogen antigen are different variants of a monkeypox H3L protein antigen, wherein the different variants of the monkeypox H3L protein antigen are derived from different strains of monkeypox. In some aspects, the first pathogen antigen and the second pathogen antigen are different variants of a monkeypox L1R protein antigen, wherein the different variants of the monkeypox L1R protein antigen are derived from different strains of monkeypox. In some aspects, the at least one additional antigen nucleic acid (e.g., second antigen nucleic acid) is operably linked to the first promoter through an internal ribosome entry site (IRES) sequence. In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

In some aspects, the single pathogen protein or first and/or second pathogen protein is a bacterial antigen selected from the group consisting of a Yersinia pestis antigen, a Mycobacterium tuberculosis antigen, antigenic fragments thereof, and any combinations thereof. In some aspects, the Yersinia pestis antigen is a Yersinia pestis capsular antigen. In some aspects, the Yersinia pestis capsular antigen is F1-Ag or virulence antigen (V-Ag). In some aspects, the Mycobacterium tuberculosis antigen is an Apa antigen, an HP65 antigen, a rAg85A antigen, any antigenic fragments thereof, or any combinations thereof.

In some aspects, the single pathogen protein or first and/or second pathogen protein and/or third pathogen protein is a viral antigen selected from the group consisting of: an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a orthopoxvirus antigen, a monkeypox antigen, a vaccinia antigen, a smallpox antigen, a Epstein bar virus antigen, a nipha virus antigen, a varicella-zoster virus antigen, antigenic fragments thereof, or any combinations thereof. In some aspects, the enterovirus antigen is an enterovirus 71 (E71) antigen, a coxsackievirus (Cox) protein antigen, antigenic fragments thereof, or any combinations thereof. In some aspects, the E71 antigen is an E71-VP1 antigen, a glutathione S-transferase (GST)-tagged E71-VP1 antigen, antigenic fragments thereof, or any combinations thereof. In some aspects, the Cox protein antigen is GST-tagged Cox protein antigen. In some aspects, the HSV antigen is an HSV-1 envelope antigen, an HSV-2 envelope antigen, an HSV-2 surface glycoprotein antigen, antigenic fragments thereof, or any combinations thereof. In some aspects, the HSV-2 surface glycoprotein antigen is a gB2 antigen, a gC2 antigen, a gD2 antigen, a gE2 antigen, or antigenic fragments thereof, or any combinations thereof. In some aspects, the HIV antigen is an Env antigen, a Gag antigen, a Nef antigen, a Pol antigen, antigenic fragments thereof, and or combinations thereof. In some aspects, the HPV antigen is a minor capsid protein L2 antigen. In some aspects, the minor capsid protein L2 antigen comprises one or more epitope domains (amino acids 10-36 and/or amino acids 65-89) of minor capsid protein L2. In some aspects, the HPV antigen is a human papillomavirus type 16 Regulatory protein E2 antigen, a human papillomavirus type 16 Protein E6 antigen, a human papillomavirus type 16 Protein E7 antigen, a human papillomavirus type 18 Regulatory protein E2 antigen, a human papillomavirus type 18 Protein E6 antigen, a human papillomavirus type 18 Protein E7 antigen, a human papillomavirus type 6a Regulatory protein E2 antigen, a human papillomavirus type 6a Protein E6 antigen, a human papillomavirus type 6a Protein E7 antigen, a human papillomavirus 11 Regulatory protein E2 antigen, a human papillomavirus 11 Protein E6 antigen, a human papillomavirus 11 Protein E7 antigen, antigenic fragments thereof, or any combinations thereof. In some aspects, the HCV antigen is a nonstructural 3 (NS3) antigen, a hepatitis C virus genotype 1a Genome polyprotein antigen, a hepatitis C virus genotype 1b Genome polyprotein antigen, a hepatitis C virus genotype 2a Genome polyprotein antigen, a hepatitis C virus genotype 3a Genome polyprotein antigen, antigenic fragments thereof, or any combinations thereof. In some aspects, the RSV antigen is an F antigen, a G antigen, antigenic fragments thereof, or any combinations thereof. In some aspects, the Dengue virus antigen is an E protein antigen, an E protein domain III (EDIII) antigen, a non-structural protein 1 (NS1) antigen, a DEN-80E antigen, antigenic fragments thereof, or any combinations thereof. In some aspects, the Ebola virus antigen is a spike glycoprotein (GB) antigen, a VP24 antigen, a VP40 antigen, a nucleoprotein (NP) antigen, a VP30 antigen, a VP35 antigen, antigenic fragments thereof, or any combinations thereof. In some aspects the Zika virus antigen is an envelope domain III antigen, a CKD antigen, antigenic fragments thereof, or any combinations thereof. In some aspects, the Chikungunya virus antigen is an E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), antigenic fragments thereof, or any combinations thereof. In some aspects, the measles virus antigen is a hemagglutinin protein MV-H antigen, a fusion protein MV-F antigen, antigenic fragments thereof, or any combinations thereof. In some aspects, the MERS-CoV antigen is a spike (S) protein antigen, an antigen from the receptor-binding domain of the S protein, an antigen from the membrane fusion domain of the S protein, antigenic fragments thereof, or any combinations thereof. In some aspects, the SARS-CoV antigen is a spike (S) protein antigen, an antigen from the receptor binding domain of the S protein, an antigen from the membrane fusion domain of the S protein, an envelope (E) protein antigen, an M protein antigen, antigenic fragments thereof, or any combinations thereof. In some aspects, the monkeypox antigen is a A35R protein antigen, a H3L protein antigen, a monkeypox L1R protein antigen, antigenic fragments thereof, or any combinations thereof. In some aspects, the Epstein-Barr virus is an Epstein-Barr virus (strain B95-8) nuclear antigen 1 antigen, an Epstein-Barr virus (strain B95-8) Envelope glycoprotein B antigen, an Epstein-Barr virus (strain B95-8) Envelope glycoprotein H antigen, an Epstein-Barr virus (strain B95-8) Envelope glycoprotein GP350 antigen, an Epstein-Barr virus (strain B95-8) Latent membrane protein 1 antigen, an Epstein-Barr virus (strain B95-8) Latent membrane protein 2 antigen, antigenic fragments thereof, or any combinations thereof. In some aspects, the Vaccinia virus is a Vaccinia virus (strain Western Reserve) Protein A27 antigen, a Vaccinia virus (strain Western Reserve) EEV membrane phosphoglycoprotein antigen, a Vaccinia virus B5R (Fragment) antigen, a Vaccinia virus Envelope protein H3 antigen, a Vaccinia virus (strain Western Reserve) IMV membrane protein antigen, antigenic fragments thereof, or any combinations thereof. In some aspects, the Nipah virus is a Nipah virus Fusion glycoprotein FO antigen, a Nipah virus Glycoprotein G antigen, antigenic fragments thereof, or any combinations thereof. In some aspects, the Varicella-zoster virus is a Varicella-zoster virus (strain Dumas) Envelope glycoprotein E antigen, antigenic fragments thereof, or any combinations thereof.

In some aspects, the single pathogen protein or first and/or second pathogen protein comprises one or more influenza virus antigens from any influenza virus type or subtype. In some aspects, the one or more influenza virus antigens are selected from the group consisting of: an influenza virus hemagglutinin (HA) antigen, an influenza virus neuraminidase (NA) antigen, an influenza virus matrix-2 (M2) protein antigen, antigenic fragments thereof, and any combination thereof. In some aspects, the one or more influenza virus antigens are derived from influenza virus type A, type B, type C, type D, or any combination thereof. In some aspects, the one or more influenza virus antigens are derived from influenza virus type A. In some aspects, the one or more influenza virus antigens derived from influenza virus type A have (a) a HA subtype selected from H1 through H18 or any combination thereof and (b) a NA subtype selected from N1 through N11 or any combination thereof. In some aspects, the one or more influenza virus antigens derived from influenza virus type A, subtype H1N1; influenza virus type A, subtype H2N2; influenza virus type A, subtype H3N2; influenza virus type A, subtype H5N1; influenza virus type A, subtype H7N7; influenza virus type A, subtype H7N9; influenza virus type A, subtype H9N2; or any combination thereof. In some aspects, the one or more influenza virus antigens are derived from influenza virus type A, subtype H1N1; influenza virus type A, subtype H3N2; or the combination thereof. In some aspects, the one or more influenza virus antigens are derived from influenza virus type B. In some aspects, the first pathogen protein comprises one or more SARS-CoV-2 antigens or antigenic fragments thereof disclosed herein, and the second pathogen protein comprises one or more influenza virus antigens or antigenic fragments thereof disclosed herein. In some aspects, the first pathogen protein comprises one or more monkeypox antigens or antigenic fragments thereof disclosed herein, and the second pathogen protein comprises one or more influenza virus antigens or antigenic fragments thereof disclosed herein. In some aspects, the pathogen protein is a parasite antigen, wherein the parasite antigen is a protozoan antigen. In some aspects, the pathogen protein is a parasite antigen selected from the group consisting of a Toxoplasma gondii antigen, a Plasmodium falciparum antigen, antigenic fragments thereof, and any combinations thereof. In some aspects, the Toxoplasma gondii antigen is antigen MIC8. In some aspects, the Plasmodium falciparum antigen is a SERA5 polypeptide antigen, a circumsporozite protein antigen, antigenic fragments thereof, or any combinations thereof. In some aspects, the pathogen protein is a parasite antigen, wherein the parasite antigen is a parasitic or pathogenic fungus antigen. In some aspects, the parasitic or pathogenic fungus antigen is selected from the group consisting of a Candida spp. antigen (e.g., a Candida albicans antigen, a Candida glabrata antigen, a Candida parapsilosis antigen, a Candida tropicalis antigen, a Candida lusitaniae antigen, a Candida krusei antigen), a Pneumocystis spp. antigen, a Malassezia spp. antigen (e.g., a Malassezia furfur antigen), an Aspergillus fumigatus antigen, a Cryptococcus spp. antigen (e.g., a Cryptococcus neoformans antigen, a Cryptococcus gattii antigen), a Histoplasma capsulatum antigen, a Blastomyces dermatitidis antigen, a Paracoccidioides spp. antigen (e.g., a Paracoccidioides brasiliensis antigen, a Paracoccidioides lutzii antigen), a Coccidioides spp. antigen (e.g., a Coccidioides immitis antigen, a Coccidioides posadasii antigen), a Penicillium marneffei antigen, a Sporothrix schenckii antigen, a Trichosporon asahii antigen, a Fusarium spp. antigen (e.g., a Fusarium solanum antigen, a Fusarium oxysporum antigen), a Nectria spp. antigen, a Pseudoallescheria boydii antigen, a Cladophialphora bantianum antigen, a Ramichloridium spp. antigen, a Dactylaria gallopava antigen, an Exophiala spp. antigen (e.g., an Exophiala jeanselmei antigen, an Exophiala dermatitidis antigen), a Curvularia spp. antigen, a Bipolaris spp. antigen, an Alternaria spp. antigen, a Lacazia loboi antigen, a Conidiobolus spp. antigen (e.g., a Conidiobolus coronatus antigen, a Conidiobolus incongruus antigen), and any combination thereof.

In some aspects, the present disclosure is directed to a polynucleotide (e.g., multicistronic DNA or multicistronic mRNA) comprising: (a) an antigen nucleic acid (e.g., first antigen nucleic acid) which encodes a SARS-CoV-2 spike (S) protein or an antigenic fragment thereof, and (b) a nucleic acid encoding an immune modifier protein. In some aspects, the present disclosure is directed to a polynucleotide (e.g., multicistronic DNA or multicistronic mRNA) comprising: (a) an antigen nucleic acid (e.g., first antigen nucleic acid) which encodes a monkeypox A35R protein, a monkeypox H3L protein, a monkeypox L1R protein, or an antigenic fragment thereof, and (b) a nucleic acid encoding an immune modifier protein. In some aspects, the antigen nucleic acid (e.g., first antigen nucleic acid) is operably linked to a first promoter. In some aspects, the polynucleotide comprises two or more nucleic acids encoding an immune modifier protein. In some aspects, each of the nucleic acids encoding an immune modifier protein encodes a different immune modifier protein.

The polynucleotides (e.g., expression vector) disclosed herein can further comprise: (c) at least one additional antigen nucleic acid (e.g., second antigen nucleic acid) which encodes a SARS-CoV-2 protein, a monkeypox protein, or an antigenic fragment thereof. In some aspects, the SARS-CoV-2 protein or antigenic fragment thereof is selected from the group consisting of: a SARS-CoV-2 membrane (M) protein or an antigenic fragment thereof, a SARS-CoV-2 envelope (E) protein or an antigenic fragment thereof, a SARS-CoV-2 nucleocapsid (N) protein or an antigenic fragment thereof, and any combination thereof. In some aspects, the antigen nucleic acid (e.g., first antigen nucleic acid) encodes a SARS-CoV-2 protein or an antigenic fragment thereof and the at least one additional antigen nucleic acid (e.g., second antigen nucleic acid) encodes a SARS-CoV-2 protein or an antigenic fragment thereof from a different strain of SARS-CoV-2. In some aspects, the antigen nucleic acid (e.g., first antigen nucleic acid) encodes a SARS-CoV-2 S protein or an antigenic fragment thereof and the at least one additional antigen nucleic acid (e.g., second antigen nucleic acid) encodes a SARS-CoV-2 S protein or an antigenic fragment thereof from a different strain of SARS-CoV-2. In some aspects, the at least one additional antigen nucleic acid (e.g., second antigen nucleic acid) is operably linked to the first promoter through an internal ribosome entry site (IRES) sequence. In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41. In some aspects, the second antigen nucleic acid is operably linked to a second promoter.

The polynucleotides (e.g., expression vector) disclosed herein can further comprise: (d) at least one additional antigen nucleic acid (e.g., a third antigen nucleic acid) which encodes a SARS-CoV-2 protein, a monkeypox protein, or an antigenic fragment thereof. In some aspects, the SARS-CoV-2 protein or antigenic fragment thereof is selected from the group consisting of: a SARS-CoV-2 membrane (M) protein or an antigenic fragment thereof, a SARS-CoV-2 envelope (E) protein or an antigenic fragment thereof, a SARS-CoV-2 nucleocapsid (N) protein or an antigenic fragment thereof, and any combination thereof. In some aspects, the third antigen nucleic acid is operably linked to a third promoter.

The polynucleotides (e.g., expression vector) disclosed herein can further comprise one or more second promoters. In some aspects, the at least one additional antigen nucleic acid (e.g., second antigen nucleic acid) is operably linked to the one or more second promoters. In some aspects, one or more nucleic acids encoding an immune modifier protein is operably linked to the one or more second promoters. In some aspects, one or more of the nucleic acids encoding an immune modifier protein is operably linked to the first promoter or the one or more second promoters through an internal ribosome entry site (IRES) sequence. In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

In some aspects, the first promoter or the one or more second promoters is selected from the group consisting of: a cytomegalovirus (CMV) promoter (SEQ ID NO: 31), a Rouse sarcoma virus (RSV) promoter, a Moloney murine leukemia virus (Mo-MuLV) long terminal repeat (LTR) promoter, a human ubiquitin C promoter, a mammalian elongation factor 1 (EF1) promoter, a human elongation factor 1α/Human T cell Leukemia Virus Type 1 Long Terminal Repeat (hEF1/HTLV) promoter, a cytokeratin 18 (CK18) promoter, a cytokeratin 19 (CK19) promoter, a simian virus 40 (SV40) promoter (SEQ ID NO: 32), a murine U6 promoter, a skeletal α-actin promoter, a β-actin promoter, a murine phosphoglycerate kinase 1 (PGK1) promoter, a human PGK1 promoter, a CMV enhancer/chicken β-actin (CAG) promoter (SEQ ID NO: 33), and any combination thereof. In some aspects, the one or more second promoters is the CMV promoter. In some aspects, the one or more second promoters is a mammalian EF1 promoter. In some aspects, the mammalian EF1 promoter is a hEF1-HTLV promoter (SEQ ID NO: 38).

In some aspects, each of the nucleic acids which encodes an immune modifier protein is under the control of a promoter selected from the group consisting of a CMV promoter, an RSV promoter, a Mo-MuLV LTR promoter, a mammalian EF 1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF 1 promoter is a hEF1-HTLV promoter.

In some aspects, each of the at least one additional antigen nucleic acid (e.g., second antigen nucleic acid)s is under the control of a promoter selected from the group consisting of a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF 1 promoter is a hEF1-HTLV promoter.

In some aspects, the immune modifier protein is selected from the group consisting of: interleukin (IL) 2 (IL-2), IL-12 p35, IL-12 p40, IL-12 p70, IL-15, IL-18, tumor necrosis factor alpha (TNFα), granulocyte-macrophage colony-stimulating factor (GM-CSF), interferon (IFN) a (IFN-α), IFN-β, a chemokine, major histocompatibility complex (MHC) class I (MHC I), MHC class II (MHC II), human leukocyte antigen (HLA)-DR isotype (HLA-DR), CD80, CD86, and any combination thereof. In some aspects, the chemokine is selected from the group consisting of: C—C motif chemokine ligand (CCL) 3 (CCL3), CCL4, CCL5, CCL21, CCL28, C—X—C motif chemokine ligand (CXCL) 10 (CXCL10), and any combination thereof.

In some aspects, the immune modifier protein is an interleukin, e.g., IL-12. IL-12 is composed of four alpha helices. It is a heterodimeric cytokine encoded by two separate genes, IL-12A (p35) and IL-12B (p40). The active heterodimer (referred to as p70), and a homodimer of p40 are formed following protein synthesis. In some aspects, the immune modifier protein is an IL-12 heterodimer (IL-12 p70) or an IL-12 homodimer (e.g., IL-12 p35 or IL-12 p40).

In some aspects, the IL-12 p35 immune modifier protein comprises an amino acid sequence having at least 70%, at least 75%, at least 80%, at least 85%, at least 90%, at least 95%, at least 99%, or 100% sequence identity to SEQ ID NO: 43 (mouse IL-12 p35) or SEQ ID NO: 47 (human IL-12 p35). In some aspects, the IL-12 p40 immune modifier protein comprises an amino acid sequence having at least 70%, at least 75%, at least 80%, at least 85%, at least 90%, at least 95%, at least 99%, or 100% sequence identity to SEQ ID NO: 45 (mouse IL-12 p40) or SEQ ID NO: 49 (human IL-12 p40).

In some aspects, the nucleic acid encoding IL-12 p35 has at least 70%, at least 75%, at least 80%, at least 85%, at least 90%, at least 95%, at least 99%, or 100% sequence identity to SEQ ID NO: 42 (nucleic acid sequence encoding mouse IL-12 p35) or SEQ ID NO: 46 (nucleic acid sequence encoding human IL-12 p35). In some aspects, the nucleic acid encoding IL-12 p40 has at least 70%, at least 75%, at least 80%, at least 85%, at least 90%, at least 95%, at least 99%, or 100% sequence identity to SEQ ID NO: 44 (nucleic acid sequence encoding mouse IL-12 p40) or SEQ ID NO: 48 (nucleic acid sequence encoding human IL-12 p40).

In some aspects, the immune modifier protein is a viral protein (e.g., SARS-CoV-2 non-structural protein 1 (Nsp1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the immune modifier protein is selected from the group consisting of SARS-CoV-2 Nsp 1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, and any combination thereof.

In some aspects, the immune modifier protein comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety.

In some aspects, polynucleotides encoding an immune modifier protein comprises a combination (i) a nucleic acid encoding an interleukin, and (ii) a nucleic acid encoding a major histocompatibility complex and/or a chemokine.

In some aspects, the polynucleotides encoding an immune modifier protein comprises a nucleic acid encoding IL-12 p35, a nucleic acid encoding IL-12 p40, or the combination thereof. In some aspects, the nucleic acid encoding IL-12 p35 has at least 70%, at least 75%, at least 80%, at least 85%, at least 90%, at least 95%, at least 99%, or 100% sequence identity to SEQ ID NO: 42 (nucleic acid sequence encoding mouse IL-12 p35) or SEQ ID NO: 46 (nucleic acid sequence encoding human IL-12 p35). In some aspects, the nucleic acid encoding IL-12 p40 has at least 70%, at least 75%, at least 80%, at least 85%, at least 90%, at least 95%, at least 99%, or 100% sequence identity to SEQ ID NO: 44 (nucleic acid sequence encoding mouse IL-12 p40) or SEQ ID NO: 48 (nucleic acid sequence encoding human IL-12 p40). In some aspects, the nucleic acid encoding an immune modifier protein further comprises a nucleic acid encoding MHC I, a nucleic acid encoding MHC II, or the combination thereof.

In some aspects, the polynucleotides encoding an immune modifier protein comprises a nucleic acid encoding IL-12, a nucleic acid encoding IL-15, or the combination thereof.

In some aspects, the polynucleotides encoding an immune modifier protein comprises a combination of a nucleic acid encoding IL-12 and a nucleic acid encoding IL-15.

In some aspects, the polynucleotides encoding an immune modifier protein comprises a nucleic acid encoding IL-2, a nucleic acid encoding IL-15, or the combination thereof. In some aspects, the nucleic acid encoding an immune modifier protein further comprises a nucleic acid encoding MHC I, a nucleic acid encoding MHC II, a nucleic acid encoding CCL3, a nucleic acid encoding CCL4, any the combination thereof.

In some aspects, the polynucleotides encoding an immune modifier comprises a nucleic acid encoding CCL3, a nucleic acid encoding CCL4, or the combination thereof.

The antigen nucleic acid (e.g., first antigen nucleic acid) of the polynucleotides disclosed herein can encode a SARS-CoV-2 full length polypeptide or antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the antigen nucleic acid (e.g., first antigen nucleic acid) of the polynucleotide encodes a polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the antigen nucleic acid (e.g., first antigen nucleic acid) of the polynucleotide encodes a polypeptide comprising the amino acid sequence of SEQ ID NO: 2 or SEQ ID NO: 4. In some aspects, the antigen nucleic acid (e.g., first antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3.

In some aspects, the at least one additional antigen nucleic acid (e.g., second antigen nucleic acid) of the polynucleotides disclosed herein can encode a SARS-CoV-2 full length polypeptide or antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the at least one additional antigen nucleic acid (e.g., second antigen nucleic acid) of the polynucleotide encodes a polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the at least one additional antigen nucleic acid (e.g., second antigen nucleic acid) of the polynucleotide encodes a polypeptide comprising the amino acid sequence of SEQ ID NO: 2 or SEQ ID NO: 4. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3.

In some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the antigen nucleic acid (e.g., first antigen nucleic acid) of the polynucleotide encodes a polypeptide comprising the amino acid sequence of SEQ ID NO: 2 or SEQ ID NO: 4, wherein the polypeptide comprises one or more mutations (i.e., one or more substitutions, deletions, insertions, or any combination thereof).

In some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the at least one additional antigen nucleic acid (e.g., second antigen nucleic acid) of the polynucleotide encodes a polypeptide comprising the amino acid sequence of SEQ ID NO: 2 or SEQ ID NO: 4, wherein the polypeptide comprises one or more mutations (i.e., one or more substitutions, deletions, insertions, or any combination thereof).

In some aspects, the antigen nucleic acid (e.g., first antigen nucleic acid) of the polynucleotides disclosed herein can encode the receptor binding domain (RBD) of the SARS-Cov-2 S protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the antigen nucleic acid (e.g., first antigen nucleic acid) of the polynucleotide encodes a polypeptide having at least 70%, at least 75%, at least 80%, at least 85%, at least 90%, at least 95%, or at least 99% sequence identity to the amino acid sequence of SEQ ID NO: 6. In some aspects, the antigen nucleic acid (e.g., first antigen nucleic acid) of the polynucleotide encodes a polypeptide comprising the amino acid sequence of SEQ ID NO: 6. In some aspects, the antigen nucleic acid (e.g., first antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 5.

In some aspects, the at least one additional antigen nucleic acid (e.g., second antigen nucleic acid) of the polynucleotides disclosed herein can encode the receptor binding domain (RBD) of the SARS-Cov-2 S protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the at least one additional antigen nucleic acid (e.g., second antigen nucleic acid) of the polynucleotide encodes a polypeptide having at least 70%, at least 75%, at least 80%, at least 85%, at least 90%, at least 95%, or at least 99% sequence identity to the amino acid sequence of SEQ ID NO: 6. In some aspects, the at least one additional antigen nucleic acid (e.g., second antigen nucleic acid) of the polynucleotide encodes a polypeptide comprising the amino acid sequence of SEQ ID NO: 6. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 5.

In some aspects, the antigen nucleic acid (e.g., first antigen nucleic acid) of the polynucleotides disclosed herein can encode the RBD of the SARS-Cov-2 S protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the antigen nucleic acid (e.g., first antigen nucleic acid) of the polynucleotide encodes a polypeptide comprising the amino acid sequence of SEQ ID NO: 6, wherein the polypeptide comprises one or more mutations (i.e., one or more substitutions, deletions, insertions, or any combination thereof).

In some aspects, the at least one additional antigen nucleic acid (e.g., second antigen nucleic acid) of the polynucleotides disclosed herein can encode the RBD of the SARS-Cov-2 S protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the at least one additional antigen nucleic acid (e.g., second antigen nucleic acid) of the polynucleotide encodes a polypeptide comprising the amino acid sequence of SEQ ID NO: 6, wherein the polypeptide comprises one or more mutations (i.e., one or more substitutions, deletions, insertions, or any combination thereof).

In some aspects, the antigen nucleic acid (e.g., first antigen nucleic acid) of the polynucleotides disclosed herein can encode the S1 subunit of the SARS-Cov-2 S protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the antigen nucleic acid (e.g., first antigen nucleic acid) encodes a polypeptide having at least 70%, at least 75%, at least 80%, at least 85%, at least 90%, at least 95%, or at least 99% sequence identity to the amino acid sequence of SEQ ID NO: 40. In some aspects, the antigen nucleic acid (e.g., first antigen nucleic acid) of the polynucleotide encodes a polypeptide comprising the amino acid sequence of SEQ ID NO: 40. In some aspects, the antigen nucleic acid (e.g., first antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 39.

In some aspects, the antigen nucleic acid (e.g., first antigen nucleic acid) of the polynucleotides disclosed herein can encode the RSV F protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 67. In some aspects, the antigen nucleic acid (e.g., first antigen nucleic acid) encodes a polypeptide having at least 70%, at least 75%, at least 80%, at least 85%, at least 90%, at least 95%, or at least 99% sequence identity to the amino acid sequence of SEQ ID NO: 67. In some aspects, the antigen nucleic acid (e.g., first antigen nucleic acid) of the polynucleotide encodes a polypeptide comprising the amino acid sequence of SEQ ID NO: 67. In some aspects, the antigen nucleic acid (e.g., first antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 66.

In some aspects, the antigen nucleic acid (e.g., first antigen nucleic acid) of the polynucleotides disclosed herein can encode the CMV gB protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 69. In some aspects, the antigen nucleic acid (e.g., first antigen nucleic acid) encodes a polypeptide having at least 70%, at least 75%, at least 80%, at least 85%, at least 90%, at least 95%, or at least 99% sequence identity to the amino acid sequence of SEQ ID NO: 69. In some aspects, the antigen nucleic acid (e.g., first antigen nucleic acid) of the polynucleotide encodes a polypeptide comprising the amino acid sequence of SEQ ID NO: 69. In some aspects, the antigen nucleic acid (e.g., first antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 68.

In some aspects, the antigen nucleic acid (e.g., first antigen nucleic acid) of the polynucleotides disclosed herein can encode the H1N1 2007 Brisbane HA protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 71. In some aspects, the antigen nucleic acid (e.g., first antigen nucleic acid) encodes a polypeptide having at least 70%, at least 75%, at least 80%, at least 85%, at least 90%, at least 95%, or at least 99% sequence identity to the amino acid sequence of SEQ ID NO: 71. In some aspects, the antigen nucleic acid (e.g., first antigen nucleic acid) of the polynucleotide encodes a polypeptide comprising the amino acid sequence of SEQ ID NO: 71. In some aspects, the antigen nucleic acid (e.g., first antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 70.

In some aspects, the antigen nucleic acid (e.g., first antigen nucleic acid or single antigen nucleic acid) of the polynucleotides disclosed herein can encode the Omicron BA.1 protein or an antigenic fragment thereof. For example, in some aspects, the antigen nucleic acid (e.g., first antigen nucleic acid or single antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 72. In some aspects, the antigen nucleic acid (e.g., first antigen nucleic acid or single antigen nucleic acid) encodes a polypeptide having at least 70%, at least 75%, at least 80%, at least 85%, at least 90%, at least 95%, or at least 99% sequence identity to the amino acid sequence of SEQ ID NO: 72. In some aspects, the antigen nucleic acid (e.g., first antigen nucleic acid) of the polynucleotide encodes a polypeptide comprising the amino acid sequence of SEQ ID NO: 72.

In some aspects, the antigen nucleic acid (e.g., first antigen nucleic acid or single antigen nucleic acid) of the polynucleotides disclosed herein can encode the Omicron BA.2 protein or an antigenic fragment thereof. For example, in some aspects, the antigen nucleic acid (e.g., first antigen nucleic acid or single antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 73. In some aspects, the antigen nucleic acid (e.g., first antigen nucleic acid or single antigen nucleic acid) encodes a polypeptide having at least 70%, at least 75%, at least 80%, at least 85%, at least 90%, at least 95%, or at least 99% sequence identity to the amino acid sequence of SEQ ID NO: 73. In some aspects, the antigen nucleic acid (e.g., first antigen nucleic acid or single antigen nucleic acid) of the polynucleotide encodes a polypeptide comprising the amino acid sequence of SEQ ID NO: 73.

In some aspects, the antigen nucleic acid (e.g., first antigen nucleic acid or single antigen nucleic acid) of the polynucleotides disclosed herein can encode the SARS-CoV-2 Delta variant protein or an antigenic fragment thereof. For example, in some aspects, the antigen nucleic acid (e.g., first antigen nucleic acid or single antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 74. In some aspects, the antigen nucleic acid (e.g., first antigen nucleic acid or single antigen nucleic acid) encodes a polypeptide having at least 70%, at least 75%, at least 80%, at least 85%, at least 90%, at least 95%, or at least 99% sequence identity to the amino acid sequence of SEQ ID NO: 74. In some aspects, the antigen nucleic acid (e.g., first antigen nucleic acid or single antigen nucleic acid) of the polynucleotide encodes a polypeptide comprising the amino acid sequence of SEQ ID NO: 74.

In some aspects, the antigen nucleic acid (e.g., first antigen nucleic acid or single antigen nucleic acid) of the polynucleotides disclosed herein can encode the monkeypox A35R protein or an antigenic fragment thereof. For example, in some aspects, the antigen nucleic acid (e.g., first antigen nucleic acid or single antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 75. In some aspects, the antigen nucleic acid (e.g., first antigen nucleic acid or single antigen nucleic acid) encodes a polypeptide having at least 70%, at least 75%, at least 80%, at least 85%, at least 90%, at least 95%, or at least 99% sequence identity to the amino acid sequence of SEQ ID NO: 75. In some aspects, the antigen nucleic acid (e.g., first antigen nucleic acid or single antigen nucleic acid) of the polynucleotide encodes a polypeptide comprising the amino acid sequence of SEQ ID NO: 75.

In some aspects, the antigen nucleic acid (e.g., first antigen nucleic acid or single antigen nucleic acid) of the polynucleotides disclosed herein can encode the monkeypox H3L protein or an antigenic fragment thereof. For example, in some aspects, the antigen nucleic acid (e.g., first antigen nucleic acid or single antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 76. In some aspects, the antigen nucleic acid (e.g., first antigen nucleic acid or single antigen nucleic acid) encodes a polypeptide having at least 70%, at least 75%, at least 80%, at least 85%, at least 90%, at least 95%, or at least 99% sequence identity to the amino acid sequence of SEQ ID NO: 76. In some aspects, the antigen nucleic acid (e.g., first antigen nucleic acid or single antigen nucleic acid) of the polynucleotide encodes a polypeptide comprising the amino acid sequence of SEQ ID NO: 76.

In some aspects, the antigen nucleic acid (e.g., first antigen nucleic acid or single antigen nucleic acid) of the polynucleotides disclosed herein can encode the monkeypox L1R protein or an antigenic fragment thereof. For example, in some aspects, the antigen nucleic acid (e.g., first antigen nucleic acid or single antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 77. In some aspects, the antigen nucleic acid (e.g., first antigen nucleic acid or single antigen nucleic acid) encodes a polypeptide having at least 70%, at least 75%, at least 80%, at least 85%, at least 90%, at least 95%, or at least 99% sequence identity to the amino acid sequence of SEQ ID NO: 77. In some aspects, the antigen nucleic acid (e.g., first antigen nucleic acid or single antigen nucleic acid) of the polynucleotide encodes a polypeptide comprising the amino acid sequence of SEQ ID NO: 77.

In some aspects, the antigen nucleic acid (e.g., first antigen nucleic acid) of the polynucleotides disclosed herein can encode the SARS-CoV-2 BA.2.75.2 variant Spike protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 115. In some aspects, the antigen nucleic acid (e.g., first antigen nucleic acid) encodes a polypeptide having at least 70%, at least 75%, at least 80%, at least 85%, at least 90%, at least 95%, or at least 99% sequence identity to the amino acid sequence of SEQ ID NO: 115. In some aspects, the antigen nucleic acid (e.g., first antigen nucleic acid) of the polynucleotide encodes a polypeptide comprising the amino acid sequence of SEQ ID NO: 115. In some aspects, the antigen nucleic acid (e.g., first antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 114.

In some aspects, the antigen nucleic acid (e.g., first antigen nucleic acid) of the polynucleotides disclosed herein can encode the SARS-CoV-2 Omicron 22E (BQ.1) variant Spike protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 117. In some aspects, the antigen nucleic acid (e.g., first antigen nucleic acid) encodes a polypeptide having at least 70%, at least 75%, at least 80%, at least 85%, at least 90%, at least 95%, or at least 99% sequence identity to the amino acid sequence of SEQ ID NO: 117. In some aspects, the antigen nucleic acid (e.g., first antigen nucleic acid) of the polynucleotide encodes a polypeptide comprising the amino acid sequence of SEQ ID NO: 117. In some aspects, the antigen nucleic acid (e.g., first antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 116.

In some aspects, the antigen nucleic acid (e.g., first antigen nucleic acid) of the polynucleotides disclosed herein can encode the SARS-CoV-2 beta variant Spike protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 127. In some aspects, the antigen nucleic acid (e.g., first antigen nucleic acid) encodes a polypeptide having at least 70%, at least 75%, at least 80%, at least 85%, at least 90%, at least 95%, or at least 99% sequence identity to the amino acid sequence of SEQ ID NO: 127. In some aspects, the antigen nucleic acid (e.g., first antigen nucleic acid) of the polynucleotide encodes a polypeptide comprising the amino acid sequence of SEQ ID NO: 127. In some aspects, the antigen nucleic acid (e.g., first antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 126.

In some aspects, the antigen nucleic acid (e.g., first antigen nucleic acid) of the polynucleotides disclosed herein can encode the SARS-CoV-2 omicron BA.5 variant Spike protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 121. In some aspects, the antigen nucleic acid (e.g., first antigen nucleic acid) encodes a polypeptide having at least 70%, at least 75%, at least 80%, at least 85%, at least 90%, at least 95%, or at least 99% sequence identity to the amino acid sequence of SEQ ID NO: 121. In some aspects, the antigen nucleic acid (e.g., first antigen nucleic acid) of the polynucleotide encodes a polypeptide comprising the amino acid sequence of SEQ ID NO: 121. In some aspects, the antigen nucleic acid (e.g., first antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 120.

In some aspects, the antigen nucleic acid (e.g., first antigen nucleic acid) of the polynucleotides disclosed herein can encode the SARS-CoV-2 BQ.1 nucleocapsid protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 123. In some aspects, the antigen nucleic acid (e.g., first antigen nucleic acid) encodes a polypeptide having at least 70%, at least 75%, at least 80%, at least 85%, at least 90%, at least 95%, or at least 99% sequence identity to the amino acid sequence of SEQ ID NO: 123. In some aspects, the antigen nucleic acid (e.g., first antigen nucleic acid) of the polynucleotide encodes a polypeptide comprising the amino acid sequence of SEQ ID NO: 123. In some aspects, the antigen nucleic acid (e.g., first antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 122.

In some aspects, the antigen nucleic acid (e.g., first antigen nucleic acid) of the polynucleotides disclosed herein can encode the SARS-CoV-2 XBB1.5 variant Spike protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 125. In some aspects, the antigen nucleic acid (e.g., first antigen nucleic acid) encodes a polypeptide having at least 70%, at least 75%, at least 80%, at least 85%, at least 90%, at least 95%, or at least 99% sequence identity to the amino acid sequence of SEQ ID NO: 125. In some aspects, the antigen nucleic acid (e.g., first antigen nucleic acid) of the polynucleotide encodes a polypeptide comprising the amino acid sequence of SEQ ID NO: 125. In some aspects, the antigen nucleic acid (e.g., first antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 124.

In some aspects, the at least one additional antigen nucleic acid (e.g., second antigen nucleic acid) of the polynucleotides disclosed herein can encode the S1 subunit of the SARS-Cov-2 S protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 40. In some aspects, the at least one additional antigen nucleic acid (e.g., second antigen nucleic acid) of the polynucleotide encodes a polypeptide comprising the amino acid sequence of SEQ ID NO: 40. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 39.

In some aspects, the at least one additional antigen nucleic acid (e.g., second antigen nucleic acid) of the polynucleotides disclosed herein can encode the RSV F protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 67. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 67. In some aspects, the at least one additional antigen nucleic acid (e.g., second antigen nucleic acid) of the polynucleotide encodes a polypeptide comprising the amino acid sequence of SEQ ID NO: 67. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 66.

In some aspects, the at least one additional antigen nucleic acid (e.g., second antigen nucleic acid) of the polynucleotides disclosed herein can encode the CMV gB protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 69. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 69. In some aspects, the at least one additional antigen nucleic acid (e.g., second antigen nucleic acid) of the polynucleotide encodes a polypeptide comprising the amino acid sequence of SEQ ID NO: 69. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 68.

In some aspects, the at least one additional antigen nucleic acid (e.g., second antigen nucleic acid) of the polynucleotides disclosed herein can encode the H1N1 2007 Brisbane HA protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 71. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 71. In some aspects, the at least one additional antigen nucleic acid (e.g., second antigen nucleic acid) of the polynucleotide encodes a polypeptide comprising the amino acid sequence of SEQ ID NO: 71. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 70.

In some aspects, the at least one additional antigen nucleic acid (e.g., second antigen nucleic acid) of the polynucleotides disclosed herein can encode the Omicron BA.1 protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 72. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 72. In some aspects, the at least one additional antigen nucleic acid (e.g., second antigen nucleic acid) of the polynucleotide encodes a polypeptide comprising the amino acid sequence of SEQ ID NO: 72.

In some aspects, the at least one additional antigen nucleic acid (e.g., second antigen nucleic acid) of the polynucleotides disclosed herein can encode the Omicron BA.2 protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 73. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 73. In some aspects, the at least one additional antigen nucleic acid (e.g., second antigen nucleic acid) of the polynucleotide encodes a polypeptide comprising the amino acid sequence of SEQ ID NO: 73.

In some aspects, the at least one additional antigen nucleic acid (e.g., second antigen nucleic acid) of the polynucleotides disclosed herein can encode the SARS-CoV-2 Delta variant protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 74. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 74. In some aspects, the at least one additional antigen nucleic acid (e.g., second antigen nucleic acid) of the polynucleotide encodes a polypeptide comprising the amino acid sequence of SEQ ID NO: 74.

In some aspects, the at least one additional antigen nucleic acid (e.g., second antigen nucleic acid) of the polynucleotides disclosed herein can encode the SARS-CoV-2 BA.2.75.2 spike protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 115. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 115. In some aspects, the at least one additional antigen nucleic acid (e.g., second antigen nucleic acid) of the polynucleotide encodes a polypeptide comprising the amino acid sequence of SEQ ID NO: 115. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 114.

In some aspects, the at least one additional antigen nucleic acid (e.g., second antigen nucleic acid) of the polynucleotides disclosed herein can encode the SARS-CoV-2 omicron 22E (BQ.1) variant spike protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 117. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 117. In some aspects, the at least one additional antigen nucleic acid (e.g., second antigen nucleic acid) of the polynucleotide encodes a polypeptide comprising the amino acid sequence of SEQ ID NO: 117. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 116.

In some aspects, the at least one additional antigen nucleic acid (e.g., second antigen nucleic acid) of the polynucleotides disclosed herein can encode the SARS-CoV-2 beta variant spike protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 127. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 127. In some aspects, the at least one additional antigen nucleic acid (e.g., second antigen nucleic acid) of the polynucleotide encodes a polypeptide comprising the amino acid sequence of SEQ ID NO: 127. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 126.

In some aspects, the at least one additional antigen nucleic acid (e.g., second antigen nucleic acid) of the polynucleotides disclosed herein can encode the SARS-CoV-2 omicron BA.5 variant spike protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 121. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 121. In some aspects, the at least one additional antigen nucleic acid (e.g., second antigen nucleic acid) of the polynucleotide encodes a polypeptide comprising the amino acid sequence of SEQ ID NO: 121. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 120.

In some aspects, the at least one additional antigen nucleic acid (e.g., second antigen nucleic acid) of the polynucleotides disclosed herein can encode the SARS-CoV-2 omicron BQ.1 nucleocapsid protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 123. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 123. In some aspects, the at least one additional antigen nucleic acid (e.g., second antigen nucleic acid) of the polynucleotide encodes a polypeptide comprising the amino acid sequence of SEQ ID NO: 123. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 122.

In some aspects, the at least one additional antigen nucleic acid (e.g., second antigen nucleic acid) of the polynucleotides disclosed herein can encode the SARS-CoV-2 omicron XBB1.5 variant spike protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 125. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 125. In some aspects, the at least one additional antigen nucleic acid (e.g., second antigen nucleic acid) of the polynucleotide encodes a polypeptide comprising the amino acid sequence of SEQ ID NO: 125. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 124.

In some aspects, the at least one additional antigen nucleic acid (e.g., second antigen nucleic acid) of the polynucleotides disclosed herein can encode the monkeypox A35R protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 75. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 75. In some aspects, the at least one additional antigen nucleic acid (e.g., second antigen nucleic acid) of the polynucleotide encodes a polypeptide comprising the amino acid sequence of SEQ ID NO: 75.

In some aspects, the at least one additional antigen nucleic acid (e.g., second antigen nucleic acid) of the polynucleotides disclosed herein can encode the monkeypox H3L protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 76. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 76. In some aspects, the at least one additional antigen nucleic acid (e.g., second antigen nucleic acid) of the polynucleotide encodes a polypeptide comprising the amino acid sequence of SEQ ID NO: 76.

In some aspects, the at least one additional antigen nucleic acid (e.g., second antigen nucleic acid) of the polynucleotides disclosed herein can encode the monkeypox L1R protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 77. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 77. In some aspects, the at least one additional antigen nucleic acid (e.g., second antigen nucleic acid) of the polynucleotide encodes a polypeptide comprising the amino acid sequence of SEQ ID NO: 77.

In some aspects, the antigen nucleic acid (e.g., first antigen nucleic acid) of the polynucleotides disclosed herein can encode the S1 subunit of the SARS-Cov-2 S protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the antigen nucleic acid (e.g., first antigen nucleic acid) of the polynucleotide encodes a polypeptide comprising the amino acid sequence of SEQ ID NO: 40, wherein the polypeptide comprises one or more mutations (i.e., one or more substitutions, deletions, insertions, or any combination thereof).

In some aspects, the at least one additional antigen nucleic acid (e.g., second antigen nucleic acid) of the polynucleotides disclosed herein can encode the S1 subunit of the SARS-Cov-2 S protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the at least one additional antigen nucleic acid (e.g., second antigen nucleic acid) of the polynucleotide encodes a polypeptide comprising the amino acid sequence of SEQ ID NO: 40, wherein the polypeptide comprises one or more mutations (i.e., one or more substitutions, deletions, insertions, or any combination thereof).

In some aspects, the antigen nucleic acid (e.g., first antigen nucleic acid) of the polynucleotides disclosed herein can encode RSV F protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 67, wherein the contiguous amino acids of SEQ ID NO: 67 comprise one or more mutations (i.e., one or more substitutions, deletions, insertions, or any combination thereof). In some aspects, the antigen nucleic acid (e.g., first antigen nucleic acid) of the polynucleotide encodes a polypeptide comprising the amino acid sequence of SEQ ID NO: 67, wherein the polypeptide comprises one or more mutations (i.e., one or more substitutions, deletions, insertions, or any combination thereof).

In some aspects, the antigen nucleic acid (e.g., first antigen nucleic acid) of the polynucleotides disclosed herein can encode CMV gB protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 69, wherein the contiguous amino acids of SEQ ID NO: 69 comprise one or more mutations (i.e., one or more substitutions, deletions, insertions, or any combination thereof). In some aspects, the antigen nucleic acid (e.g., first antigen nucleic acid) of the polynucleotide encodes a polypeptide comprising the amino acid sequence of SEQ ID NO: 69, wherein the polypeptide comprises one or more mutations (i.e., one or more substitutions, deletions, insertions, or any combination thereof).

In some aspects, the antigen nucleic acid (e.g., first antigen nucleic acid) of the polynucleotides disclosed herein can encode the H1N1 2007 Brisbane HA protein or an antigenic fragment thereof. For example, in some aspects, the antigen nucleic acid (e.g., first antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 71, wherein the contiguous amino acids of SEQ ID NO: 71 comprise one or more mutations (i.e., one or more substitutions, deletions, insertions, or any combination thereof). In some aspects, the antigen nucleic acid (e.g., first antigen nucleic acid) of the polynucleotide encodes a polypeptide comprising the amino acid sequence of SEQ ID NO: 71, wherein the polypeptide comprises one or more mutations (i.e., one or more substitutions, deletions, insertions, or any combination thereof).

In some aspects, the antigen nucleic acid (e.g., first antigen nucleic acid or single antigen nucleic acid) of the polynucleotides disclosed herein can encode the Omicron BA.1 protein or an antigenic fragment thereof. For example, in some aspects, the antigen nucleic acid (e.g., first antigen nucleic acid or single antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 72, wherein the contiguous amino acids of SEQ ID NO: 72 comprise one or more mutations (i.e., one or more substitutions, deletions, insertions, or any combination thereof). In some aspects, the antigen nucleic acid (e.g., first antigen nucleic acid or single antigen nucleic acid) of the polynucleotide encodes a polypeptide comprising the amino acid sequence of SEQ ID NO: 72, wherein the polypeptide comprises one or more mutations (i.e., one or more substitutions, deletions, insertions, or any combination thereof).

In some aspects, the antigen nucleic acid (e.g., first antigen nucleic acid or single antigen nucleic acid) of the polynucleotides disclosed herein can encode the Omicron BA.2 protein or an antigenic fragment thereof. For example, in some aspects, the antigen nucleic acid (e.g., first antigen nucleic acid or single antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 73, wherein the contiguous amino acids of SEQ ID NO: 73 comprise one or more mutations (i.e., one or more substitutions, deletions, insertions, or any combination thereof). In some aspects, the antigen nucleic acid (e.g., first antigen nucleic acid) of the polynucleotide encodes a polypeptide comprising the amino acid sequence of SEQ ID NO: 73, wherein the polypeptide comprises one or more mutations (i.e., one or more substitutions, deletions, insertions, or any combination thereof).

In some aspects, the antigen nucleic acid (e.g., first antigen nucleic acid or single antigen nucleic acid) of the polynucleotides disclosed herein can encode the SARS-CoV-2 Delta variant protein or an antigenic fragment thereof. For example, in some aspects, the antigen nucleic acid (e.g., first antigen nucleic acid or single antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 74, wherein the contiguous amino acids of SEQ ID NO: 74 comprise one or more mutations (i.e., one or more substitutions, deletions, insertions, or any combination thereof). In some aspects, the antigen nucleic acid (e.g., first antigen nucleic acid) of the polynucleotide encodes a polypeptide comprising the amino acid sequence of SEQ ID NO: 74, wherein the polypeptide comprises one or more mutations (i.e., one or more substitutions, deletions, insertions, or any combination thereof).

In some aspects, the antigen nucleic acid (e.g., first antigen nucleic acid or single antigen nucleic acid) of the polynucleotides disclosed herein can encode the monkeypox A35R protein or an antigenic fragment thereof. For example, in some aspects, the antigen nucleic acid (e.g., first antigen nucleic acid or single antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 75, wherein the contiguous amino acids of SEQ ID NO: 75 comprise one or more mutations (i.e., one or more substitutions, deletions, insertions, or any combination thereof). In some aspects, the antigen nucleic acid (e.g., first antigen nucleic acid) of the polynucleotide encodes a polypeptide comprising the amino acid sequence of SEQ ID NO: 75, wherein the polypeptide comprises one or more mutations (i.e., one or more substitutions, deletions, insertions, or any combination thereof).

In some aspects, the antigen nucleic acid (e.g., first antigen nucleic acid or single antigen nucleic acid) of the polynucleotides disclosed herein can encode the monkeypox H3L protein or an antigenic fragment thereof. For example, in some aspects, the antigen nucleic acid (e.g., first antigen nucleic acid or single antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 76, wherein the contiguous amino acids of SEQ ID NO: 76 comprise one or more mutations (i.e., one or more substitutions, deletions, insertions, or any combination thereof). In some aspects, the antigen nucleic acid (e.g., first antigen nucleic acid) of the polynucleotide encodes a polypeptide comprising the amino acid sequence of SEQ ID NO: 76, wherein the polypeptide comprises one or more mutations (i.e., one or more substitutions, deletions, insertions, or any combination thereof).

In some aspects, the antigen nucleic acid (e.g., first antigen nucleic acid or single antigen nucleic acid) of the polynucleotides disclosed herein can encode the monkeypox L1R protein or an antigenic fragment thereof. For example, in some aspects, the antigen nucleic acid (e.g., first antigen nucleic acid or single antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 77, wherein the contiguous amino acids of SEQ ID NO: 77 comprise one or more mutations (i.e., one or more substitutions, deletions, insertions, or any combination thereof). In some aspects, the antigen nucleic acid (e.g., first antigen nucleic acid) of the polynucleotide encodes a polypeptide comprising the amino acid sequence of SEQ ID NO: 77, wherein the polypeptide comprises one or more mutations (i.e., one or more substitutions, deletions, insertions, or any combination thereof).

In some aspects, the first antigen nucleic acid, the second antigen nucleic acid, or the third antigen nucleic acid of the polynucleotides disclosed herein can encode the SARS-CoV-2 nucleocapsid protein or an antigenic fragment thereof. For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 123. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 123. In some aspects, the at least one additional antigen nucleic acid (e.g., second antigen nucleic acid) of the polynucleotide encodes a polypeptide comprising the amino acid sequence of SEQ ID NO: 123. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 122.

In some aspects, the one or more mutations in the SARS-CoV-2 full-length S protein, the RBD of the SARS-Cov-2 S protein, the S1 subunit of the SARS-CoV-2 S protein, or antigenic fragments thereof comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety.

In some aspects, the one or more mutations in the SARS-CoV-2 full-length S protein, the RBD of the SARS-Cov-2 S protein, the S1 subunit of the SARS-CoV-2 S protein, or antigenic fragments thereof are selected from: AM1-S13, S12P, S13I, L5F, L18F, T19R, T20N, P26S, Q52R, A67V, AH69-V70, G75V, T76I, D80A, T95I, R102I, AD119-F120, C136Y, D138Y, AF140, AL141-Y144, AY144, Y144S, Y145N, AH146, N148S, K150R, K150E, K150T, K150Q, S151P, W152C, E154K, AE156-F157, F157L, F157A, R158G, R190S, AI210, D215G, A222V, AL241-S243, AL242-L244, AA243-L244, AR246-G252, R246I, 11-amino acid residue insertion between Y248 and L249, D253G, D253N, R346K, V367F, E406W, K417E, K417V, K417N, K417T, N439K, K444A, K444R, K444N, K444Q, V445A, V445E, G447A, N450D, L452R, L452Q, Y453F, L455F, N460I, S477G, S477N, S477R, T478I, T478K, V483I, E484K, E484Q, G485R, F486A, F486V, F486L, N487A, F490S, Q493E, Q493K, S494P, N501Y, A570D, Q613H, D614G, H655Y, Q677H, A678-679, A681-682, A681-684, A682-685, P681H, P681R, R682S, R682A, R682Q, R683S, R683G, R683Q, R685G, R685Q, I692V, A701V, T716I, F817P, T859N, F888L, A892P, A899P, A942P, D950N, S982A, K986P, V987P, T1027I, Q1071H, D1118H, V1176F, M1229I, AC1253-T1273, AC1254-T1273, AK1255-T1273, D1257A, E1258A, D1259A, D1260A, E1262A, K1269A, H1271K, T1273A, or any combination thereof, wherein the amino acid locations correspond to SEQ ID NO: 2 or SEQ ID NO: 4.

In some aspects, the one or more mutations in the SARS-CoV-2 full-length S protein, or antigenic fragments thereof are selected from A67V, AH69-V70, T95I, G142-Y145D, N211I, Δ212I, 214EPEins, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, or any combination thereof, wherein the amino acid locations correspond to SEQ ID NO: 2 or SEQ ID NO: 4.

In some aspects, the one or more mutations in the SARS-CoV-2 full-length S protein, or antigenic fragments thereof are selected from T19I, LPPA24-27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K, or any combination thereof, wherein the amino acid locations correspond to SEQ ID NO: 2 or SEQ ID NO: 4. In some aspects, the one or more mutations comprise one or more mutations in the N-terminal signal peptide, which corresponds to amino acids 1-13 of SEQ ID NO: 2 or SEQ ID NO: 4. In some aspects, the one or more mutations in the N-terminal signal peptide is AM1-S13, wherein the amino acid locations correspond to SEQ ID NO: 2 or SEQ ID NO: 4.

In some aspects, the one or mutations comprise one or more mutations in the C-terminus of the full-length SARS-CoV-2 S protein. In some aspects, the one or more mutations in the C-terminus of the full-length SARS-CoV-2 S protein comprise one or more mutations in the C-terminal endoplasmic reticulum (ER) retention peptide, which corresponds to amino acids 1254-1273 of SEQ ID NO: 2 or SEQ ID NO: 4. In some aspects, the one or more mutations in the C-terminal ER retention peptide are selected from D1257A, E1258A, D1259A, D1260A, E1262A, K1269A, H1271K, T1273A, or any combination thereof, wherein the amino acid locations correspond to SEQ ID NO: 2 or SEQ ID NO: 4. In some aspects, the one or more mutations in the C-terminal ER retention peptide comprise D1257A+E1258A+D1259A+D1260A+E1262A (i.e., a D/E to A mutant), wherein the amino acid locations correspond to SEQ ID NO: 2 or SEQ ID NO: 4. In some aspects, the one or mutations in the C-terminal ER retention peptide is AC1253-T1273, AC1254-T1273, or AK1255-T1273.

In some aspects, the one or more mutations comprise K986P+V987P (i.e., a S-2P mutation), wherein the amino acid locations correspond to SEQ ID NO: 2 or SEQ ID NO: 4. In some aspects, the one or more mutations comprise F817P+A892P+A899P+A942P (i.e., a hexa-proline S mutation), wherein the amino acid locations correspond to SEQ ID NO: 2 or SEQ ID NO: 4. In some aspects, the one or more mutations comprise one or more mutations in the 681-PRRAR/SVA-688 S1/S2 furin cleavage site, wherein the amino acid locations correspond to SEQ ID NO: 2 or SEQ ID NO: 4. In some aspects, the one or more mutations in the 681-PRRAR/SVA-688 S1/S2 furin cleavage site are: (a) R682S+R683S (i.e., a SSAR mutation), (b) A681-684 (i.e., a APRRA mutation), (c) A678-679+A681-682, (d) R682A+R683G+R685G (i.e., a 682-AGAG-685 mutation), (e) R682Q+R683Q+R685Q, (f) R682S+R685G, or (g) A682-685 (i.e., a ARRAR mutation), wherein the amino acid locations correspond to SEQ ID NO: 2 or SEQ ID NO: 4.

In some aspects, the one or more mutations comprise: (a) F817P+A892P+A899P+A942P (i.e., a hexa-proline S mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation), (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682Q+R683Q+R685Q and (b) K986P+V987P (i.e., a S-2P mutation). In some aspects, the one or more mutations comprise: : (a) R682Q+R683Q+R685Q, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682S+R685G and (b) K986P+V987P (i.e., a S-2P mutation), wherein the amino acid locations correspond to SEQ ID NO: 2 or SEQ ID NO: 4. In some aspects, the one or more mutations comprise: (a) R682S+R685G, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4.

The at least one additional antigen nucleic acid (e.g., second antigen nucleic acid) of the polynucleotides disclosed herein can encode a SARS-CoV-2 M protein or an antigenic fragment thereof, a SARS-CoV-2 E protein or an antigenic fragment thereof, a SARS-CoV-2 N protein or an antigenic fragment thereof, or any combination thereof. In some aspects, the antigen nucleic acid (e.g., first antigen nucleic acid) and the at least one additional antigen nucleic acid (e.g., second antigen nucleic acid) of the polynucleotides encode SARS-CoV-2 proteins or antigenic fragments thereof from different strains of SARS-CoV-2. In some aspects, the antigen nucleic acid (e.g., first antigen nucleic acid) and the at least one additional antigen nucleic acid (e.g., second antigen nucleic acid) of the polynucleotides encode variants of the same SARS-CoV-2 protein or antigenic fragment thereof, wherein the variants of the same SARS-CoV-2 protein or antigenic fragment thereof are derived from different strains of SARS-CoV-2. In some aspects, the antigen nucleic acid (e.g., first antigen nucleic acid) and the at least one additional antigen nucleic acid (e.g., second antigen nucleic acid) of the polynucleotides encode variants of a SARS-CoV-2 S protein or antigenic fragment thereof, wherein the variants of the SARS-CoV-2 S protein or antigenic fragment thereof are derived from different strains of SARS-CoV-2.

For example, in some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the at least one additional antigen nucleic acid (e.g., second antigen nucleic acid) of the polynucleotide encodes a polypeptide comprising the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 7, SEQ ID NO: 9, SEQ ID NO: 11, SEQ ID NO: 13, SEQ ID NO: 15, SEQ ID NO: 17, SEQ ID NO: 19, or SEQ ID NO: 131.

In some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20 comprise one or more mutations selected from A2S, F28L, I48V, V70L, 182T, M84T, or any combination thereof, wherein the amino acid locations correspond to SEQ ID NO: 8. In some aspects, the at least one additional antigen nucleic acid (e.g., second antigen nucleic acid) of the polynucleotide encodes a polypeptide comprising the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the polypeptide comprises one or more mutations selected from A2S, F28L, I48V, V70L, I82T, M84T, or any combination thereof, wherein the amino acid locations correspond to SEQ ID NO: 8.

In some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 55, at least 60, at least 65, at least 70, or at least 75 contiguous amino acids of SEQ ID NO: 22, SEQ ID NO: 24, or SEQ ID NO: 26. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 22, SEQ ID NO: 24, or SEQ ID NO: 26. In some aspects, the at least one additional antigen nucleic acid (e.g., second antigen nucleic acid) of the polynucleotide encodes a polypeptide comprising the amino acid sequence of SEQ ID NO: 22, SEQ ID NO: 24, or SEQ ID NO: 26. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 21, SEQ ID NO: 23, or SEQ ID NO: 25.

In some aspects, the at least one additional antigen nucleic acid (e.g., second antigen nucleic acid) of the polynucleotide encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 28. In some aspects, the at least one additional antigen nucleic acid (e.g., second antigen nucleic acid) encodes a polypeptide having at least 70%, at least 75%, at least 80%, at least 85%, at least 90%, at least 95%, or at least 99% sequence identity to the amino acid sequence of SEQ ID NO: 28. In some aspects, the at least one additional antigen nucleic acid (e.g., second antigen nucleic acid) of the polynucleotide encodes a polypeptide comprising the amino acid sequence of SEQ ID NO: 28. In some aspects, the at least one additional antigen nucleic acid (e.g., second antigen nucleic acid) of the polynucleotide comprises a nucleic acid sequence having at least 70%, at least 75%, at least 80%, at least 85%, at least 90%, at least 95%, at least 99%, or 100% sequence identity to SEQ ID NO: 27.

In some aspects, the present disclosure is directed to a polynucleotide (e.g., multicistronic DNA or multicistronic mRNA) comprising an antigen nucleic acid (e.g., first antigen nucleic acid) which encodes a first pathogen protein or an antigenic fragment thereof, wherein the antigen nucleic acid (e.g., first antigen nucleic acid) is operably linked to a first promoter. In some aspects, the antigen nucleic acid (e.g., first antigen nucleic acid) which encodes a first pathogen protein is selected from the group consisting of a viral protein, a bacterial protein, a parasite protein, and any antigenic fragment thereof.

In some aspects, the polynucleotide further comprises at least one additional antigen nucleic acid (e.g., second antigen nucleic acid) which encodes a second pathogen protein or an antigenic fragment thereof. In some aspects, the at least one additional antigen nucleic acid (e.g., second antigen nucleic acid) which encodes a second pathogen protein is selected from the group consisting of a viral protein, a bacterial protein, a parasite protein, and any antigenic fragment thereof.

In some aspects, the pathogen protein or first pathogen protein and/or the second pathogen protein is/are selected from the group consisting of a Yersiniapestis antigen, a Mycobacterium tuberculosis antigen, an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a orthopoxvirus antigen, a monkeypox antigen, a vaccinia antigen, a smallpox antigen, a Epstein bar virus antigen, a nipha virus antigen, a varicella-zoster virus antigen, a Clostridioides difficile antigen, a Streptococcus pneumonia antigen, a Neisseria meningitides antigen, a Toxoplasma gondii antigen, a Plasmodium falciparum: antigen, an influenza virus antigen, antigenic fragments thereof, and any combinations thereof.

In some aspects, the single pathogen protein or first pathogen protein and/or the second pathogen protein is/are selected from the group consisting of: a Yersiniapestis F1-Ag, a Yersinia pestis V-Ag, a Mycobacterium tuberculosis Apa antigen, a Mycobacterium tuberculosis HP65 antigen, a Mycobacterium tuberculosis rAg85A antigen, an E71 VP1 antigen, a GST-tagged E71-VP1 antigen, a Cox protein antigen, a GST-tagged Cox protein antigen, an HSV-1 envelope antigen, an HSV-2 envelope antigen, an HSV-2 gB2 antigen, an HSV-2 gC2 antigen, an HSV-2 gD2 antigen, an HSV-2 gE2 antigen, an HIV Env antigen, an HIV Gag antigen, an HIV Nef antigen, an HIV Pol antigen, an HPV minor capsid protein L2 antigen, an HCV NS3 antigen, a RSV F antigen, a RSV G antigen, a Dengue virus E protein antigen, a Dengue virus EDIII antigen, a Dengue virus NS1 antigen, a Dengue virus DEN-80E antigen, an Ebola virus GB antigen, an Ebola virus VP24 antigen, an Ebola virus VP40 antigen, an Ebola virus NP antigen, an Ebola virus VP30 antigen, an Ebola virus VP35 antigen, a Zika virus envelope domain III antigen, a Zika virus CKD antigen, a Chikungunya virus E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), a measles virus hemagglutinin protein MV-H antigen, a measles virus fusion protein MV-F antigen, a MERS-CoV S protein antigen, an antigen from the receptor-binding domain of the MERS-CoV S protein, an antigen from the membrane fusion domain of the MERS-CoV S protein, a SARS-CoV S protein antigen, an antigen from the receptor binding domain of the SARS-CoV S protein, an antigen from the membrane fusion domain of the SARS-CoV S protein, a SARS-CoV E protein antigen, a SARS-CoV M protein antigen, a SARS-CoV N protein, a monkeypox A35R protein antigen, a monkeypox H3L protein antigen, a monkeypox LIR protein antigen, a Clostridioides difficile 630 spore coat protein: peroxiredoxin/chitinase antigen, a Clostridioides difficile 630 flagellin C antigen, a Clostridioides difficile Surface layer protein A (Fragment) antigen, an Epstein-Barr virus (strain B95-8) nuclear antigen 1 antigen, an Epstein-Barr virus (strain B95-8) Envelope glycoprotein B antigen, an Epstein-Barr virus (strain B95-8) Envelope glycoprotein H antigen, an Epstein-Barr virus (strain B95-8) Envelope glycoprotein GP350 antigen, an Epstein-Barr virus (strain B95-8) Latent membrane protein 1 antigen, an Epstein-Barr virus (strain B95-8) Latent membrane protein 2 antigen, a Neisseria meningitides Factor H-binding protein antigen, a Neisseria meningitidis serogroup B Neisseria adhesin A antigen, a Neisseria meningitidis Neisserial heparin binding antigen antigen, a Vaccinia virus (strain Western Reserve) Protein A27 antigen, a Vaccinia virus (strain Western Reserve) EEV membrane phosphoglycoprotein antigen, a Vaccinia virus B5R (Fragment) antigen, a Vaccinia virus Envelope protein H3 antigen, a Vaccinia virus (strain Western Reserve) IMV membrane protein antigen, a Nipah virus Fusion glycoprotein FO antigen, a Nipah virus Glycoprotein G antigen, a Varicella-zoster virus (strain Dumas) Envelope glycoprotein E antigen, a human papillomavirus type 16 Regulatory protein E2 antigen, a human papillomavirus type 16 Protein E6 antigen, a human papillomavirus type 16 Protein E7 antigen, a human papillomavirus type 18 Regulatory protein E2 antigen, a human papillomavirus type 18 Protein E6 antigen, a human papillomavirus type 18 Protein E7 antigen, a human papillomavirus type 6a Regulatory protein E2 antigen, a human papillomavirus type 6a Protein E6 antigen, a human papillomavirus type 6a Protein E7 antigen, a human papillomavirus 11 Regulatory protein E2 antigen, a human papillomavirus 11 Protein E6 antigen, a human papillomavirus 11 Protein E7 antigen, a hepatitis C virus genotype 1α Genome polyprotein antigen, a hepatitis C virus genotype 1b Genome polyprotein antigen, a hepatitis C virus genotype 2a Genome polyprotein antigen, a hepatitis C virus genotype 3a Genome polyprotein antigen, a Toxoplasma gondii MIC8 antigen, a Plasmodium falciparum: SERA5 polypeptide antigen, a Plasmodium falciparum: circumsporozite protein antigen, an influenza virus hemagglutinin (HA) antigen, an influenza virus neuraminidase (NA) antigen, an influenza virus matrix-2 (M2) protein antigen, antigenic fragments thereof, and any combination thereof.

In some aspects, the antigen nucleic acid (e.g., first antigen nucleic acid) encodes a SARS CoV-2 spike (S) protein or an antigenic fragment thereof. In some aspects, the second pathogen protein or antigenic fragment thereof is selected from the group consisting of: a SARS-CoV-2 membrane (M) protein or an antigenic fragment thereof, a SARS-CoV-2 envelope (E) protein or an antigenic fragment thereof, a SARS-CoV-2 nucleocapsid (N) protein or an antigenic fragment thereof, and any combination thereof. In some aspects, the SARS-CoV-2 S protein or antigenic fragment thereof and the second pathogen protein or antigenic fragment thereof are derived from different strains of SARS-CoV-2. In some aspects, the antigen nucleic acid (e.g., first antigen nucleic acid) encodes a SARS CoV-2 S protein or antigenic fragment thereof and a second SARS CoV-2 S protein or antigenic fragment thereof from a different strain of SARS-CoV-2.

In some aspects, the at least one additional antigen nucleic acid (e.g., second antigen nucleic acid) is operably linked to the first promoter through an internal ribosome entry site (IRES) sequence. In some aspects, the IRES sequence comprises a nucleic acid sequence having at least 70%, at least 75%, at least 80%, at least 85%, at least 90%, at least 95%, at least 99%, or 100% sequence identity to SEQ ID NO: 41.

In some aspects, the polynucleotide comprises one or more second promoters. In some aspects, the at least one additional antigen nucleic acid (e.g., second antigen nucleic acid) is operably linked to the one or more second promoters.

In some aspects, the first promoter or the one or more second promoters is selected from the group consisting of: a cytomegalovirus (CMV) promoter, a Rouse sarcoma virus (RSV) promoter, a Moloney murine leukemia virus (Mo-MuLV) long terminal repeat (LTR) promoter, a human ubiquitin C promoter, a mammalian elongation factor 1 (EF1) promoter, a human elongation factor 1α/Human T cell Leukemia Virus Type 1 Long Terminal Repeat (hEF1/HTLV) promoter, a cytokeratin 18 (CK18) promoter, a cytokeratin 19 (CK19) promoter, a simian virus 40 (SV40) promoter, a murine U6 promoter, a skeletal α-actin promoter, a β-actin promoter, a murine phosphoglycerate kinase 1 (PGK1) promoter, a human PGK1 promoter, a CBA promoter, a CAG promoter, and any combination thereof. In some aspects, the mammalian EF1 promoter is a hEF1-HTLV promoter. In some aspects, the one or more second promoters is the CMV promoter.

In some aspects, the at least one additional antigen nucleic acid (e.g., second antigen nucleic acid) is under the control of a promoter selected from the group consisting of a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CBA promoter, a CAG promoter, and any combination thereof. In some aspects, the mammalian EF 1 promoter is a hEF1-HTLV promoter.

In some aspects, the third antigen nucleic acid is under the control of a promoter selected from the group consisting of a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CBA promoter, a CAG promoter, and any combination thereof. In some aspects, the mammalian EF 1 promoter is a hEF1-HTLV promoter. In some aspects, the third antigen nucleic acid is under the control of a CMV promoter.

In some aspects, the antigen nucleic acid (e.g., first antigen nucleic acid) encodes a full-length SARS-CoV-2 S protein or an antigenic fragment thereof. In some aspects, the antigen nucleic acid (e.g., first antigen nucleic acid) encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the antigen nucleic acid (e.g., first antigen nucleic acid) encodes a polypeptide comprising the amino acid sequence of SEQ ID NO: 2 or SEQ ID NO: 4. In some aspects, the antigen nucleic acid (e.g., first antigen nucleic acid) comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3. In some aspects, the antigen nucleic acid (e.g., first antigen nucleic acid) is operably linked to a mammalian EF 1 promoter. In some aspects, the mammalian EF1 promoter is a hEF1-HTLV promoter.

In some aspects, the at least one additional antigen nucleic acid (e.g., second antigen nucleic acid) encodes a full-length SARS-CoV-2 S protein or an antigenic fragment thereof. In some aspects, the at least one additional antigen nucleic acid (e.g., second antigen nucleic acid) encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the at least one additional antigen nucleic acid (e.g., second antigen nucleic acid) encodes a polypeptide comprising the amino acid sequence of SEQ ID NO: 2 or SEQ ID NO: 4. In some aspects, the at least one additional antigen nucleic acid (e.g., second antigen nucleic acid) comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3. In some aspects, the at least one additional antigen nucleic acid (e.g., second antigen nucleic acid) is operably linked to a mammalian EF 1 promoter through an IRES sequence. In some aspects, the mammalian EF 1 promoter is a hEF1-HTLV promoter.

In some aspects, the antigen nucleic acid (e.g., first antigen nucleic acid) encodes a full-length SARS-CoV-2 S protein or an antigenic fragment thereof, and wherein the at least one additional antigen nucleic acid (e.g., second antigen nucleic acid) encodes a SARS-CoV-2 membrane (M) protein or an antigenic fragment thereof. In some aspects, the antigen nucleic acid (e.g., first antigen nucleic acid) encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, and wherein the at least one additional antigen nucleic acid (e.g., second antigen nucleic acid) encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the antigen nucleic acid (e.g., first antigen nucleic acid) encodes a polypeptide comprising the amino acid sequence of SEQ ID NO: 2 or SEQ ID NO: 4, and wherein the at least one additional antigen nucleic acid (e.g., second antigen nucleic acid) encodes a polypeptide comprising the amino acid sequence of SEQ ID NO: 8, SEQ ID NO: 10 SEQ ID NO: 12, SEQ ID NO: 14, SEQ ID NO: 16, SEQ ID NO: 18, or SEQ ID NO: 20. In some aspects, the antigen nucleic acid (e.g., first antigen nucleic acid) comprises a nucleic acid sequence having at least 70%, at least 75%, at least 80%, at least 85%, at least 90%, at least 95%, at least 99%, or 100% sequence identity to SEQ ID NO: 1 or SEQ ID NO: 3, and wherein the at least one additional antigen nucleic acid (e.g., second antigen nucleic acid) comprises a nucleic acid sequence having at least 70%, at least 75%, at least 80%, at least 85%, at least 90%, at least 95%, at least 99%, or 100% sequence identity to SEQ ID NO: 7, SEQ ID NO: 9, SEQ ID NO: 11, SEQ ID NO: 13, SEQ ID NO: 15, SEQ ID NO: 17, SEQ ID NO: 19, or SEQ ID NO: 131. In some aspects, the antigen nucleic acid (e.g., first antigen nucleic acid) is operably linked to a mammalian EF 1 promoter, and wherein the at least one additional antigen nucleic acid (e.g., second antigen nucleic acid) is operably linked to a CMV promoter. In some aspects, the mammalian EF 1 promoter is a hEF1-HTLV promoter.

In some aspects, the antigen nucleic acid (e.g., first antigen nucleic acid) encodes the receptor binding domain (RBD) of the SARS-CoV-2 S protein or an antigenic fragment thereof. In some aspects, the antigen nucleic acid (e.g., first antigen nucleic acid) encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the antigen nucleic acid (e.g., first antigen nucleic acid) encodes a polypeptide comprising the amino acid sequence of SEQ ID NO: 6. In some aspects, the antigen nucleic acid (e.g., first antigen nucleic acid) comprises a nucleic acid sequence having at least 70%, at least 75%, at least 80%, at least 85%, at least 90%, at least 95%, at least 99%, or 100% sequence identity to SEQ ID NO: 5.

In some aspects, the at least one additional antigen nucleic acid (e.g., second antigen nucleic acid) encodes the receptor binding domain (RBD) of the SARS-CoV-2 S protein or an antigenic fragment thereof. In some aspects, the at least one additional antigen nucleic acid (e.g., second antigen nucleic acid) encodes at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the at least one additional antigen nucleic acid (e.g., second antigen nucleic acid) encodes a polypeptide comprising the amino acid sequence of SEQ ID NO: 6. In some aspects, the at least one additional antigen nucleic acid (e.g., second antigen nucleic acid) comprises a nucleic acid sequence having at least 70%, at least 75%, at least 80%, at least 85%, at least 90%, at least 95%, at least 99%, or 100% sequence identity to SEQ ID NO: 5.

In some aspects, the antigen nucleic acid (e.g., first antigen nucleic acid) encodes the S1 subunit of the SARS-CoV-2 S protein or an antigenic fragment thereof. In some aspects, the antigen nucleic acid (e.g., first antigen nucleic acid) encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the antigen nucleic acid (e.g., first antigen nucleic acid) encodes a polypeptide comprising the amino acid sequence of SEQ ID NO: 40. In some aspects, the antigen nucleic acid (e.g., first antigen nucleic acid) comprises a nucleic acid sequence having at least 70%, at least 75%, at least 80%, at least 85%, at least 90%, at least 95%, at least 99%, or 100% sequence identity to SEQ ID NO: 39. In some aspects, the antigen nucleic acid (e.g., first antigen nucleic acid) is operably linked to a mammalian EF1 promoter. In some aspects, the mammalian EF1 promoter is a hEF1-HTLV promoter.

In some aspects, the at least one additional antigen nucleic acid (e.g., second antigen nucleic acid) encodes the S1 subunit of the SARS-CoV-2 S protein or an antigenic fragment thereof. In some aspects, the at least one additional antigen nucleic acid (e.g., second antigen nucleic acid) encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the at least one additional antigen nucleic acid (e.g., second antigen nucleic acid) encodes a polypeptide comprising the amino acid sequence of SEQ ID NO: 40. In some aspects, the at least one additional antigen nucleic acid (e.g., second antigen nucleic acid) comprises a nucleic acid sequence having at least 70%, at least 75%, at least 80%, at least 85%, at least 90%, at least 95%, at least 99%, or 100% sequence identity to SEQ ID NO: 39. In some aspects, the at least one additional antigen nucleic acid (e.g., second antigen nucleic acid) is operably linked to a mammalian EF1 promoter through an IRES sequence. In some aspects, the mammalian EF 1 promoter is a hEF1-HTLV promoter.

In some aspects, the at least one additional antigen nucleic acid (e.g., second antigen nucleic acid) encodes at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the at least one additional antigen nucleic acid (e.g., second antigen nucleic acid) encodes a polypeptide comprising the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the at least one additional antigen nucleic acid (e.g., second antigen nucleic acid) comprises a nucleic acid sequence having at least 70%, at least 75%, at least 80%, at least 85%, at least 90%, at least 95%, at least 99%, or 100% sequence identity to SEQ ID NO: 7, SEQ ID NO: 9, SEQ ID NO: 11, SEQ ID NO: 13, SEQ ID NO: 15, SEQ ID NO: 17, SEQ ID NO: 19, or SEQ ID NO: 131. In some aspects, the at least one additional antigen nucleic acid (e.g., second antigen nucleic acid) encodes at least 8, at least 10, at least 15, at least 20, at least 25, at least 30, at least 35, at least 40, at least 45, at least 50, at least 55, at least 60, at least 65, at least 70, or at least 75 contiguous amino acids of SEQ ID NO: 22, SEQ ID NO: 24, or SEQ ID NO: 26. In some aspects, the at least one additional antigen nucleic acid (e.g., second antigen nucleic acid) encodes a polypeptide comprising the amino acid sequence of SEQ ID NO: 22, SEQ ID NO: 24, or SEQ ID NO: 26. In some aspects, the at least one additional antigen nucleic acid (e.g., second antigen nucleic acid) comprises a nucleic acid sequence having at least 70%, at least 75%, at least 80%, at least 85%, at least 90%, at least 95%, at least 99%, or 100% sequence identity to SEQ ID NO: 21, SEQ ID NO: 23, or SEQ ID NO: 25. In some aspects, the at least one additional antigen nucleic acid (e.g., second antigen nucleic acid) encodes at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 28. In some aspects, the at least one additional antigen nucleic acid (e.g., second antigen nucleic acid) encodes a polypeptide comprising the amino acid sequence of SEQ ID NO: 28. In some aspects, the at least one additional antigen nucleic acid (e.g., second antigen nucleic acid) comprises a nucleic acid sequence having at least 70%, at least 75%, at least 80%, at least 85%, at least 90%, at least 95%, at least 99%, or 100% sequence identity to SEQ ID NO: 27.

In some aspects, the polypeptide(s) encoded by the antigen nucleic acid (e.g., first antigen nucleic acid) (e.g., an antigen nucleic acid (e.g., first antigen nucleic acid) encoding a SARS-CoV-2 full-length S protein, the RBD of a SARS-Cov-2 S protein, or the S1 subunit of a SARS-CoV-2 S protein) and/or the at least one additional antigen nucleic acid (e.g., second antigen nucleic acid) (e.g., an antigen nucleic acid (e.g., first antigen nucleic acid) encoding a SARS-CoV-2 full-length S protein, the RBD of a SARS-Cov-2 S protein, or the S1 subunit of a SARS-CoV-2 S protein) comprise one or more mutations. In some aspects, the one or more mutations in the polypeptide(s) encoded by the antigen nucleic acid (e.g., first antigen nucleic acid) or the at least one additional antigen nucleic acid (e.g., second antigen nucleic acid) comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety.

In some aspects, the one or more mutations in the polypeptide(s) encoded by the antigen nucleic acid (e.g., first antigen nucleic acid) and/or the at least one additional antigen nucleic acid (e.g., second antigen nucleic acid) are selected from: AM1-S13, S12P, S13I, L5F, L18F, T19R, T20N, P26S, Q52R, A67V, AH69-V70, G75V, T76I, D80A, T95I, R102I, AD119-F120, C136Y, D138Y, AF140, AL141-Y144, AY144, Y144S, Y145N, AH146, N148S, K150R, K150E, K150T, K150Q, S151P, W152C, E154K, AE156-F157, F157L, F157A, R158G, R190S, AI210, D215G, A222V, AL241-S243, AL242-L244, AA243-L244, AR246-G252, R246I, 11-amino acid residue insertion between Y248 and L249, D253G, D253N, R346K, V367F, E406W, K417E, K417V, K417N, K417T, N439K, K444A, K444R, K444N, K444Q, V445A, V445E, G447A, N450D, L452R, L452Q, Y453F, L455F, N460I, S477G, S477N, S477R, T478I, T478K, V483I, E484K, E484Q, G485R, F486A, F486V, F486L, N487A, F490S, Q493E, Q493K, S494P, N501Y, A570D, Q613H, D614G, H655Y, Q677H, A678-679, A681-682, A681-684, A682-685, P681H, P681R, R682S, R682A, R682Q, R683S, R683G, R683Q, R685G, R685Q, I692V, A701V, T716I, F817P, T859N, F888L, A892P, A899P, A942P, D950N, S982A, K986P, V987P, T1027I, Q1071H, D1118H, V1176F, M1229I, AC1253-T1273, AC1254-T1273, AK1255-T1273, D1257A, E1258A, D1259A, D1260A, E1262A, K1269A, H1271K, T1273A, or any combination thereof, wherein the amino acid locations correspond to SEQ ID NO: 2 or SEQ ID NO: 4.

In some aspects, the one or more mutations in the polypeptide(s) encoded by the antigen nucleic acid (e.g., first antigen nucleic acid) and/or the at least one additional antigen nucleic acid (e.g., second antigen nucleic acid) are selected from: A67V, ΔH69-V70, T95I, G142D, ΔV143, ΔY144, Y145D, N211I, Δ212I, 214EPEins, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, or any combination thereof, wherein the amino acid locations correspond to SEQ ID NO: 2 or SEQ ID NO: 4.

In some aspects, the one or more mutations in the polypeptide(s) encoded by the antigen nucleic acid (e.g., first antigen nucleic acid) and/or the at least one additional antigen nucleic acid (e.g., second antigen nucleic acid) are selected from: T19I, LPPA24-27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K, or any combination thereof, wherein the amino acid locations correspond to SEQ ID NO: 2 or SEQ ID NO: 4.

In some aspects, the one or more mutations in the polypeptide(s) encoded by the antigen nucleic acid (e.g., first antigen nucleic acid) and/or the at least one additional antigen nucleic acid (e.g., second antigen nucleic acid) comprise one or more mutations in the N-terminal signal peptide of the full-length SARS-CoV-2 S protein, which corresponds to amino acids 1-13 of SEQ ID NO: 2 or SEQ ID NO: 4. In some aspects, the one or more mutations in the N-terminal signal peptide is AM1-S13, wherein the amino acid locations correspond to SEQ ID NO: 2 or SEQ ID NO: 4.

In some aspects, the one or mutations in the polypeptide(s) encoded by the antigen nucleic acid (e.g., first antigen nucleic acid) and/or the at least one additional antigen nucleic acid (e.g., second antigen nucleic acid) comprise one or more mutations in the C-terminus of the full-length SARS-CoV-2 S protein. In some aspects, the one or more mutations in the C-terminus of the full-length SARS-CoV-2 S protein comprise one or more mutations in the C-terminal endoplasmic reticulum (ER) retention peptide, which corresponds to amino acids 1254-1273 of SEQ ID NO: 2 or SEQ ID NO: 4. In some aspects, the one or more mutations in the C-terminal ER retention peptide are selected from D1257A, E1258A, D1259A, D1260A, E1262A, K1269A, H1271K, T1273A, or any combination thereof, wherein the amino acid locations correspond to SEQ ID NO: 2 or SEQ ID NO: 4. In some aspects, the one or more mutations in the C-terminal ER retention peptide comprise D1257A+E1258A+D1259A+D1260A+E1262A (i.e., a D/E to A mutant), wherein the amino acid locations correspond to SEQ ID NO: 2 or SEQ ID NO: 4. In some aspects, the one or mutations in the C-terminal ER retention peptide is AC1253-T1273, AC1254-T1273, or AK1255-T1273.

In some aspects, the one or more mutations in the polypeptide(s) encoded by the antigen nucleic acid (e.g., first antigen nucleic acid) and/or the at least one additional antigen nucleic acid (e.g., second antigen nucleic acid) comprise K986P+V987P (i.e., a S-2P mutation), wherein the amino acid locations correspond to SEQ ID NO: 2 or SEQ ID NO: 4. In some aspects, the one or more mutations in the polypeptide(s) encoded by the antigen nucleic acid (e.g., first antigen nucleic acid) and/or the at least one additional antigen nucleic acid (e.g., second antigen nucleic acid) comprise F817P+A892P+A899P+A942P (i.e., a hexa-proline S mutation), wherein the amino acid locations correspond to SEQ ID NO: 2 or SEQ ID NO: 4. In some aspects, the one or more mutations in the polypeptide(s) encoded by the antigen nucleic acid (e.g., first antigen nucleic acid) and/or the at least one additional antigen nucleic acid (e.g., second antigen nucleic acid) comprise one or more mutations in the 681-PRRAR/SVA-688 S1/S2 furin cleavage site, wherein the amino acid locations correspond to SEQ ID NO: 2 or SEQ ID NO: 4. In some aspects, the one or more mutations in the 681-PRRAR/SVA-688 S1/S2 furin cleavage site are: (a) R682S+R683S (i.e., a SSAR mutation), (b) A681-684 (i.e., a APRRA mutation), (c) A678-679+A681-682, (d) R682A+R683G+R685G (i.e., a 682-AGAG-685 mutation), (e) R682Q+R683Q+R685Q, (f) R682S+R685G, or (g) A682-685 (i.e., a ARRAR mutation), wherein the amino acid locations correspond to SEQ ID NO: 2 or SEQ ID NO: 4.

In some aspects, the one or more mutations in the polypeptide(s) encoded by the antigen nucleic acid (e.g., first antigen nucleic acid) and/or the at least one additional antigen nucleic acid (e.g., second antigen nucleic acid) comprise: (a) F817P+A892P+A899P+A942P (i.e., a hexa-proline S mutation) and (b) K986P+V987P (i.e., a S-2P mutation), wherein the amino acid locations correspond to SEQ ID NO: 2 or SEQ ID NO: 4. In some aspects, the one or more mutations in the polypeptide(s) encoded by the antigen nucleic acid (e.g., first antigen nucleic acid) and/or the at least one additional antigen nucleic acid (e.g., second antigen nucleic acid) comprise: (a) R682A+R683G+R685G (i.e., a 682-AGAG-685 mutation) and (b) K986P+V987P (i.e., a S-2P mutation), wherein the amino acid locations correspond to SEQ ID NO: 2 or SEQ ID NO: 4. In some aspects, the one or more mutations in the polypeptide(s) encoded by the antigen nucleic acid (e.g., first antigen nucleic acid) and/or the at least one additional antigen nucleic acid (e.g., second antigen nucleic acid) comprise: (a) R682A+R683G+R685G (i.e., a 682-AGAG-685 mutation), (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations in the polypeptide(s) encoded by the antigen nucleic acid (e.g., first antigen nucleic acid) and/or the at least one additional antigen nucleic acid (e.g., second antigen nucleic acid) comprise: (a) R682Q+R683Q+R685Q and (b) K986P+V987P (i.e., a S-2P mutation). In some aspects, the one or more mutations in the polypeptide(s) encoded by the antigen nucleic acid (e.g., first antigen nucleic acid) and/or the at least one additional antigen nucleic acid (e.g., second antigen nucleic acid) comprise: : (a) R682Q +R683Q+R685Q, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations in the polypeptide(s) encoded by the antigen nucleic acid (e.g., first antigen nucleic acid) and/or the at least one additional antigen nucleic acid (e.g., second antigen nucleic acid) comprise: (a) R682S+R685G and (b) K986P+V987P (i.e., a S-2P mutation), wherein the amino acid locations correspond to SEQ ID NO: 2 or SEQ ID NO: 4. In some aspects, the one or more mutations in the polypeptide(s) encoded by the antigen nucleic acid (e.g., first antigen nucleic acid) and/or the at least one additional antigen nucleic acid (e.g., second antigen nucleic acid) comprise: (a) R682S+R685G, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4.

The polynucleotides disclosed herein can further comprise one or more post-transcriptional regulatory elements. In some aspects, the post-translational regulatory element is positioned 3′ to a coding region of the polynucleotide. Non-limiting examples of post-transcriptional regulatory elements that are useful for the present disclosure include a mutated woodchuck hepatitis virus post-transcriptional regulatory element (WPRE), microRNA binding site, DNA nuclear targeting sequence, or combinations thereof. In some aspects, the post-transcriptional regulatory element is a WPRE.

The polynucleotide can also comprise one or more polyadenylation (poly(a)) signals, which can be downstream of any protein coding sequence. Examples of polyadenylation signals include but are not limited to a SV40 poly(a) tail (SEQ ID NO: 29), LTR poly(a) tail, bovine growth hormone (bGH) poly(a) tail (SEQ ID NO: 30), human growth hormone (hGH) poly(a) tail, or human β-globin poly(a) tail. The SV40 polyadenylation signal can be a polyadenylation signal from a pCEP4 vector (Invitrogen, San Diego, CA). In some aspects, the polynucleotides disclosed herein further comprise at least one 3′ UTR poly(a) tail sequence operably linked to the antigen nucleic acid (e.g., first antigen nucleic acid), the at least one additional antigen nucleic acid (e.g., second antigen nucleic acid), the nucleic acid encoding an immune modifier protein, or any combination thereof. In some aspects, the 3′ UTR poly(a) tail sequence is a 3′ UTR SV40 poly(a) tail sequence, a 3′ UTR bovine growth hormone (bGH) poly(A) sequence, a 3′ UTR actin poly(A) tail sequence, a 3′ UTR hemoglobin poly(A) sequence, or combinations thereof.

The polynucleotides disclosed herein can further comprise at least one enhancer sequence upstream of any protein coding sequence. The enhancer can be necessary for DNA expression. In some aspects, the enhancer is a human actin enhancer, human myosin enhancer, human hemoglobin enhancer, human muscle creatine enhancer, or a viral enhancer such as one from CMV, HA, RSV or EBV. In some aspects, the enhancer is a polynucleotide function enhancer as described in U.S. Pat. Nos. 5,593,972, 5,962,428, and WO 94/016737. In some aspects, the enhancer sequence is a CMV intronic sequence or a β-actin intronic sequence. In aspects, the enhancer sequence is a SV40 enhancer sequence (SEQ ID NO: 37). In some aspects, the enhancer sequence is a CMV intron A sequence (SEQ ID NO: 128). In some aspects, the polynucleotide comprises both a SV40 enhancer sequence (SEQ ID NO: 37) and a CMV intron A sequence (SEQ ID NO: 128).

The polynucleotides disclosed herein can further comprise a leader sequence. In some aspects, the leader sequence is an IgE leader sequence. In some aspects, the leader sequence comprises a nucleic acid sequence having at least 70%, at least 75%, at least 80%, at least 85%, at least 90%, at least 95%, at least 99%, or 100% sequence identity to SEQ ID NO: 129. In some aspects, the leader sequence encodes a polypeptide having at least 70%, at least 75%, at least 80%, at least 85%, at least 90%, at least 95%, or at least 99% sequence identity to the amino acid sequence of SEQ ID NO: 130.

The polynucleotides disclosed herein can further comprise one or more inverted terminal repeats (ITRs). In some aspects, the polynucleotide comprises a first ITR and a second ITR. In some aspects, the polynucleotide comprises a first ITR, e.g., a 5′ ITR, and a second ITR, e.g., a 3′ ITR. Typically, ITRs are involved in parvovirus (e.g., adeno-associated virus (AAV)) DNA replication and rescue, or excision, from prokaryotic plasmids (Samulski et al., 1983, 1987; Senapathy et al., 1984; Gottlieb and Muzyczka, 1988). In addition, ITRs appear to be the minimum sequences required for AAV proviral integration and for packaging of AAV DNA into virions (McLaughlin et al., 1988; Samulski et al., 1989). These elements are essential for efficient multiplication of a parvovirus genome. In some aspects, the ITRs fold into a hairpin T-shaped structure. In some aspects, the ITRs fold into non-T-shaped hairpin structures, e.g., into a U-shaped hairpin structure.

In some aspects, the ITRs that are useful for the present disclosure comprise an ITR from an AAV genome. In certain aspects, the ITR is an ITR of an AAV genome selected from AAV1, AAV2, AAV3, AAV4, AAV5, AAV6, AAV7, AAV8, AAV9, AAV10, AAV 11, and any combination thereof. In some aspects, the ITR is an ITR of the AAV2 genome. In some aspects, the ITR is a synthetic sequence genetically engineered to include at its 5′ and 3′ ends ITRs derived from one or more of AAV genomes.

In some aspects, the ITR is not derived from an AAV genome. In some aspects, the ITR is an ITR of a non-AAV. In some aspects, the ITR is an ITR of a non-AAV genome from the viral family Parvoviridae selected from, but not limited to, the group consisting of Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, Penstyldensovirus and any combination thereof. In certain aspects, the ITR is derived from erythrovirus parvovirus B19 (human virus). In some aspects, the ITR is derived from a Muscovy duck parvovirus (MDPV) strain. In certain aspects, the MDPV strain is attenuated, e.g., MDPV strain FZ91-30. In some aspects, the MDPV strain is pathogenic, e.g., MDPV strain YY. In some aspects, the ITR is derived from a porcine parvovirus, e.g., porcine parvovirus U44978. In some aspects, the ITR is derived from a mice minute virus, e.g., mice minute virus U34256. In some aspects, the ITR is derived from a canine parvovirus, e.g., canine parvovirus M19296. In some aspects, the ITR is derived from a mink enteritis virus, e.g., mink enteritis virus D00765. In some aspects, the ITR is derived from a Dependoparvovirus. In certain aspects, the Dependoparvovirus is a Dependovirus Goose parvovirus (GPV) strain. In some aspects, the GPV strain is attenuated, e.g., GPV strain 82-0321V. In some aspects, the GPV strain is pathogenic, e.g., GPV strain.

The polynucleotides disclosed herein can also comprise a mammalian origin of replication (e.g., an Epstein Barr virus origin of replication) in order to maintain the vector extrachromosomally and produce multiple copies of the vector in a cell.

In some aspects, the polynucleotide is a multicistronic mRNA. In some aspects, the multicistronic mRNA comprises a 5′ cap and a 3′ UTR poly(A) tail sequence. In some aspects, the 3′ UTR poly(a) tail sequence is a 3′ UTR SV40 poly(a) tail sequence (SEQ ID NO: 29), a 3′ UTR bovine growth hormone (bGH) poly(A) sequence (SEQ ID NO: 30), a 3′ UTR actin poly(A) tail sequence, a 3′ UTR hemoglobin poly(A) sequence, or any combinations thereof. In some aspects, the multicistronic mRNA comprises a 5′ UTR and/or a 3′ UTR.

In some aspects, the polynucleotides disclosed herein can comprise: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes a first pathogen protein (e.g., a SARS-CoV-2 protein or a monkeypox protein) and is operably linked to promoter 1; and a fourth nucleotide sequence, wherein the fourth nucleotide sequence encodes a second pathogen protein (e.g., a SARS-CoV-2 protein or a monkeypox protein) and is operably linked to promoter 2. For example, in some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes a first pathogen protein (e.g., a SARS-CoV-2 protein or a monkeypox protein) and is operably linked to promoter 1; and a fourth nucleotide sequence positioned 3′ to the third nucleotide sequence, wherein the fourth nucleotide sequence encodes a second pathogen protein (e.g., a SARS-CoV-2 protein or a monkeypox protein) and is operably linked to promoter 2. In some aspects, the first, second, third, and fourth nucleotide sequences of the polynucleotide are configured as shown in FIG. 1.

In some aspects, the polynucleotides disclosed herein can comprise: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes MHC I and is operably linked to promoter Z; a fourth nucleotide sequence, wherein the fourth nucleotide sequence encodes a first pathogen protein (e.g., a SARS-CoV-2 protein or a monkeypox protein) and is operably linked to promoter 1, and a fifth nucleotide sequence, wherein the fifth nucleotide sequence encodes a second pathogen protein (e.g., a SARS-CoV-2 protein or a monkeypox protein) and is operably linked to promoter 2. For example, in some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes MHC I and is operably linked to promoter Z; a fourth nucleotide sequence positioned 3′ to the third nucleotide sequence, wherein the fourth nucleotide sequence encodes a first pathogen protein (e.g., a SARS-CoV-2 protein or a monkeypox protein) and is operably linked to promoter 1, and a fifth nucleotide sequence positioned 3′ to the fourth nucleotide sequence, wherein the fifth nucleotide sequence encodes a second pathogen protein (e.g., a SARS-CoV-2 protein or a monkeypox protein) and is operably linked to promoter 2. In some aspects, the first, second, third, fourth, and fifth nucleotide sequences of the polynucleotide are configured as shown in FIG. 2.

In some aspects, the polynucleotides disclosed herein can comprises: a first nucleotide sequence, wherein the first nucleotide sequence encodes IL-12 p35 and is operably linked to a CMV promoter; a second nucleotide sequence, wherein the second nucleotide sequence encodes IL-12 p40 and is operably linked to a CMV promoter; a third nucleotide sequence, wherein the third nucleotide sequence encodes MHC II and is operably linked to promoter Z; a fourth nucleotide sequence, wherein the fourth nucleotide sequence encodes a first pathogen protein (e.g., a SARS-CoV-2 protein or a monkeypox protein) and is operably linked to promoter 1, and a fifth nucleotide sequence, wherein the fifth nucleotide sequence encodes a second pathogen protein (e.g., a SARS-CoV-2 protein or a monkeypox protein) and is operably linked to promoter 2. For example, in some aspects, the polynucleotide comprises: a 5′ first nucleotide sequence, wherein the first nucleotide sequence encodes IL-12 p35 and is operably linked to a CMV promoter; a second nucleotide sequence positioned 3′ to the first nucleotide sequence, wherein the second nucleotide sequence encodes IL-12 p40 and is operably linked to a CMV promoter; a third nucleotide sequence positioned 3′ to the second nucleotide sequence, wherein the third nucleotide sequence encodes MHC II and is operably linked to promoter Z; a fourth nucleotide sequence positioned 3′ to the third nucleotide sequence, wherein the fourth nucleotide sequence encodes a first pathogen protein (e.g., a SARS-CoV-2 protein or a monkeypox protein) and is operably linked to promoter 1, and a fifth nucleotide sequence positioned 3′ to the fourth nucleotide sequence, wherein the fifth nucleotide sequence encodes a second pathogen protein (e.g., a SARS-CoV-2 protein or a monkeypox protein) and is operably linked to promoter 2. In some aspects, the first, second, third, fourth, and fifth nucleotide sequences of the polynucleotide are configured as shown in FIG. 3.

In some aspects, the polynucleotides disclosed herein can comprise: a first nucleotide sequence, wherein the first nucleotide sequence encodes IL-2 and is operably linked to a CMV promoter; a second nucleotide sequence, wherein the second nucleotide sequence encodes a first pathogen protein (e.g., a SARS-CoV-2 protein or a monkeypox protein) and is operably linked to promoter 1; and a third nucleotide sequence, wherein the third nucleotide sequence encodes a second pathogen protein (e.g., a SARS-CoV-2 protein or a monkeypox protein) and is operably linked to promoter 2. For example, in some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes a first pathogen protein (e.g., a SARS-CoV-2 protein or a monkeypox protein) and is operably linked to promoter 1; and a third nucleotide sequence positioned 3′ to the second nucleotide sequence, wherein the third nucleotide sequence encodes a second pathogen protein (e.g., a SARS-CoV-2 protein or a monkeypox protein) and is operably linked to promoter 2. In some aspects, the first, second, and third nucleotide sequences of the polynucleotide are configured as shown in FIG. 4.

In some aspects, the polynucleotides disclosed herein can comprise: a first nucleotide sequence, wherein the first nucleotide sequence encodes IL-2 and is operably linked to a CMV promoter; a second nucleotide sequence, wherein the second nucleotide sequence encodes MHC I and is operably linked to promoter Z; a third nucleotide sequence, wherein the third nucleotide sequence encodes a first pathogen protein (e.g., a SARS-CoV-2 protein or a monkeypox protein) and is operably linked to promoter 1; and a fourth nucleotide sequence, wherein the fourth nucleotide sequence encodes a second pathogen protein (e.g., a SARS-CoV-2 protein or a monkeypox protein) and is operably linked to promoter 2. For example, in some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes MHC I and is operably linked to promoter Z; a third nucleotide sequence positioned 3′ to the second nucleotide sequence, wherein the third nucleotide sequence encodes a first pathogen protein (e.g., a SARS-CoV-2 protein or a monkeypox protein) and is operably linked to promoter 1; and a fourth nucleotide sequence positioned 3′ to the third nucleotide sequence, wherein the fourth nucleotide sequence encodes a second pathogen protein (e.g., a SARS-CoV-2 protein or a monkeypox protein) and is operably linked to promoter 2. In some aspects, the first, second, third, and fourth nucleotide sequences of the polynucleotide are configured as shown in FIG. 5.

In some aspects, the polynucleotides disclosed herein can comprise: a first nucleotide sequence, wherein the first nucleotide sequence encodes IL-2 and is operably linked to a CMV promoter; a second nucleotide sequence, wherein the second nucleotide sequence encodes MHC II and is operably linked to promoter Z; a third nucleotide sequence, wherein the third nucleotide sequence encodes a first pathogen protein (e.g., a SARS-CoV-2 protein or a monkeypox protein) and is operably linked to promoter 1; and a fourth nucleotide sequence, wherein the fourth nucleotide sequence encodes a second pathogen protein (e.g., a SARS-CoV-2 protein or a monkeypox protein) and is operably linked to promoter 2. For example, in some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes MHC II and is operably linked to promoter Z; a third nucleotide sequence positioned 3′ to the second nucleotide sequence, wherein the third nucleotide sequence encodes a first pathogen protein (e.g., a SARS-CoV-2 protein or a monkeypox protein) and is operably linked to promoter 1; and a fourth nucleotide sequence positioned 3′ to the third nucleotide sequence, wherein the fourth nucleotide sequence encodes a second pathogen protein (e.g., a SARS-CoV-2 protein or a monkeypox protein) and is operably linked to promoter 2. In some aspects, the first, second, third, and fourth nucleotide sequences of the polynucleotide are configured as shown in FIG. 6.

In some aspects, the polynucleotides disclosed herein can comprise: a first nucleotide sequence, wherein the first nucleotide sequence encodes IL-2 and is operably linked to a CMV promoter; a second nucleotide sequence, wherein the second nucleotide sequence encodes CCL3 and is operably linked to promoter X; a third nucleotide sequence, wherein the third nucleotide sequence encodes CCL4 and is operably linked to promoter Y; a fourth nucleotide sequence, wherein the fourth nucleotide sequence encodes a first pathogen protein (e.g., a SARS-CoV-2 protein or a monkeypox protein) and is operably linked to promoter 1, and a fifth nucleotide sequence, wherein the fifth nucleotide sequence encodes a second pathogen protein (e.g., a SARS-CoV-2 protein or a monkeypox protein) and is operably linked to promoter 2. For example, in some aspects, the polynucleotide comprises: a 5′ first nucleotide sequence, wherein the first nucleotide sequence encodes IL-2 and is operably linked to a CMV promoter; a second nucleotide sequence positioned 3′ to the first nucleotide sequence, wherein the second nucleotide sequence encodes CCL3 and is operably linked to promoter X; a third nucleotide sequence positioned 3′ to the second nucleotide sequence, wherein the third nucleotide sequence encodes CCL4 and is operably linked to promoter Y; a fourth nucleotide sequence positioned 3′ to the third nucleotide sequence, wherein the fourth nucleotide sequence encodes a first pathogen protein (e.g., a SARS-CoV-2 protein or a monkeypox protein) and is operably linked to promoter 1, and a fifth nucleotide sequence positioned 3′ to the fourth nucleotide sequence, wherein the fifth nucleotide sequence encodes a second pathogen protein (e.g., a SARS-CoV-2 protein or a monkeypox protein) and is operably linked to promoter 2. In some aspects, the first, second, third, fourth, and fifth nucleotide sequences of the polynucleotide are configured as shown in FIG. 7.

In some aspects, the polynucleotides disclosed herein can comprise: a first nucleotide sequence, wherein the first nucleotide sequence encodes IL-15 and is operably linked to a CMV promoter; a second nucleotide sequence, wherein the second nucleotide sequence encodes a first pathogen protein (e.g., a SARS-CoV-2 protein or a monkeypox protein) and is operably linked to promoter 1; and a third nucleotide sequence, wherein the third nucleotide sequence encodes a second pathogen protein (e.g., a SARS-CoV-2 protein or a monkeypox protein) and is operably linked to promoter 2. For example, in some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes a first pathogen protein (e.g., a SARS-CoV-2 protein or a monkeypox protein) and is operably linked to promoter 1; and a third nucleotide sequence positioned 3′ to the second nucleotide sequence, wherein the third nucleotide sequence encodes a second pathogen protein (e.g., a SARS-CoV-2 protein or a monkeypox protein) and is operably linked to promoter 2. In some aspects, the first, second, and third nucleotide sequences of the polynucleotide are configured as shown in FIG. 8.

In some aspects, the polynucleotides disclosed herein can comprise: a first nucleotide sequence, wherein the first nucleotide sequence encodes IL-15 and is operably linked to a CMV promoter; a second nucleotide sequence, wherein the second nucleotide sequence encodes MHC I and is operably linked to promoter Z; a third nucleotide sequence, wherein the third nucleotide sequence encodes a first pathogen protein (e.g., a SARS-CoV-2 protein or a monkeypox protein) and is operably linked to promoter 1; and a fourth nucleotide sequence, wherein the fourth nucleotide sequence encodes a second pathogen protein (e.g., a SARS-CoV-2 protein or a monkeypox protein) and is operably linked to promoter 2. For example, in some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes MHC I and is operably linked to promoter Z; a third nucleotide sequence positioned 3′ to the second nucleotide sequence, wherein the third nucleotide sequence encodes a first pathogen protein (e.g., a SARS-CoV-2 protein or a monkeypox protein) and is operably linked to promoter 1; and a fourth nucleotide sequence positioned 3′ to the third nucleotide sequence, wherein the fourth nucleotide sequence encodes a second pathogen protein (e.g., a SARS-CoV-2 protein or a monkeypox protein) and is operably linked to promoter 2. In some aspects, the first, second, third, and fourth nucleotide sequences of the polynucleotide are configured as shown in FIG. 9.

In some aspects, the polynucleotides disclosed herein can comprise: a first nucleotide sequence, wherein the first nucleotide sequence encodes IL-15 and is operably linked to a CMV promoter; a second nucleotide sequence, wherein the second nucleotide sequence encodes MHC II and is operably linked to promoter Z; a third nucleotide sequence, wherein the third nucleotide sequence encodes a first pathogen protein (e.g., a SARS-CoV-2 protein or a monkeypox protein) and is operably linked to promoter 1; and a fourth nucleotide sequence, wherein the fourth nucleotide sequence encodes a second pathogen protein (e.g., a SARS-CoV-2 protein or a monkeypox protein) and is operably linked to promoter 2. For example, in some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes MHC II and is operably linked to promoter Z; a third nucleotide sequence positioned 3′ to the second nucleotide sequence, wherein the third nucleotide sequence encodes a first pathogen protein (e.g., a SARS-CoV-2 protein or a monkeypox protein) and is operably linked to promoter 1; and a fourth nucleotide sequence positioned 3′ to the third nucleotide sequence, wherein the fourth nucleotide sequence encodes a second pathogen protein (e.g., a SARS-CoV-2 protein or a monkeypox protein) and is operably linked to promoter 2. In some aspects, the first, second, third, and fourth nucleotide sequences of the polynucleotide are configured as shown in FIG. 10.

In some aspects, the polynucleotides disclosed herein can comprise: a first nucleotide sequence, wherein the first nucleotide sequence encodes IL-15 and is operably linked to a CMV promoter; a second nucleotide sequence, wherein the second nucleotide sequence encodes CCL3 and is operably linked to promoter X; a third nucleotide sequence, wherein the third nucleotide sequence encodes CCL4 and is operably linked to promoter Y; a fourth nucleotide sequence, wherein the fourth nucleotide sequence encodes a first pathogen protein (e.g., a SARS-CoV-2 protein or a monkeypox protein) and is operably linked to promoter 1, and a fifth nucleotide sequence, wherein the fifth nucleotide sequence encodes a second pathogen protein (e.g., a SARS-CoV-2 protein or a monkeypox protein) and is operably linked to promoter 2. For example, in some aspects, the polynucleotide comprises: a 5′ first nucleotide sequence, wherein the first nucleotide sequence encodes IL-15 and is operably linked to a CMV promoter; a second nucleotide sequence positioned 3′ to the first nucleotide sequence, wherein the second nucleotide sequence encodes CCL3 and is operably linked to promoter X; a third nucleotide sequence positioned 3′ to the second nucleotide sequence, wherein the third nucleotide sequence encodes CCL4 and is operably linked to promoter Y; a fourth nucleotide sequence positioned 3′ to the third nucleotide sequence, wherein the fourth nucleotide sequence encodes a first pathogen protein (e.g., a SARS-CoV-2 protein or a monkeypox protein) and is operably linked to promoter 1, and a fifth nucleotide sequence positioned 3′ to the fourth nucleotide sequence, wherein the fifth nucleotide sequence encodes a second pathogen protein (e.g., a SARS-CoV-2 protein or a monkeypox protein) and is operably linked to promoter 2. In some aspects, the first, second, third, fourth, and fifth nucleotide sequences of the polynucleotide are configured as shown in FIG. 11.

In some aspects, the polynucleotides disclosed herein can comprise: a first nucleotide sequence, wherein the first nucleotide sequence encodes CCL3 and is operably linked to promoter X; a second nucleotide sequence, wherein the second nucleotide sequence encodes CCL4 and is operably linked to promoter Y; a third nucleotide sequence, wherein the third nucleotide sequence encodes a first pathogen protein (e.g., a SARS-CoV-2 protein or a monkeypox protein) and is operably linked to promoter 1; and a fourth nucleotide sequence, wherein the fourth nucleotide sequence encodes a second pathogen protein (e.g., a SARS-CoV-2 protein or a monkeypox protein) and is operably linked to promoter 2. For example, in some aspects, the polynucleotide comprises: a 5′ first nucleotide sequence, wherein the first nucleotide sequence encodes CCL3 and is operably linked to promoter X; a second nucleotide sequence positioned 3′ to the first nucleotide sequence, wherein the second nucleotide sequence encodes CCL4 and is operably linked to promoter Y; a third nucleotide sequence positioned 3′ to the second nucleotide sequence, wherein the third nucleotide sequence encodes a first pathogen protein (e.g., a SARS-CoV-2 protein or a monkeypox protein) and is operably linked to promoter 1; and a fourth nucleotide sequence positioned 3′ to the third nucleotide sequence, wherein the fourth nucleotide sequence encodes a second pathogen protein (e.g., a SARS-CoV-2 protein or a monkeypox protein) and is operably linked to promoter 2. In some aspects, the first, second, third, and fourth nucleotide sequences of the polynucleotide are configured as shown in FIG. 12.

In some aspects, the vector constructs illustrated in any of FIGS. 1-12 can modified to replace the “Covid-19 Spike Gene” (a first nucleotide sequence encoding a SARS-CoV-2 protein) and the “Covid-19 Gene-2” (a second nucleotide sequence encoding a SARS-CoV-2 protein) with nucleotide sequences encoding any combinations of pathogen antigen or antigenic fragment thereof disclosed herein. In some aspect, the nucleotide sequences encode antigens to a virus, a bacteria or a parasite. In some aspects, the nucleotide sequences encode one or more antigens comprise one or more viral antigens, one or more bacterial antigens, or one or more parasite antigens. In some aspects the vector constructs disclosed herein can be used to express DNA or mRNA.

In some aspects, the polynucleotides disclosed herein can comprise: a nucleotide sequence, wherein the nucleotide sequence encodes a pathogen protein (e.g., a SARS-CoV-2 antigen or a monkeypox antigen) and the nucleotide sequence is operably linked to a promoter. In some aspects, the polynucleotide further comprises a nucleic acid sequence encoding one or more immune modifier proteins. In some aspects, the polynucleotide comprises a nucleic acid sequence encoding a SARS CoV-2 antigen and, optionally, a second viral antigen (e.g., a second SARS CoV-2 antigen and/or an influenza virus antigen). In some aspects, the polynucleotide comprises a nucleic acid sequence encoding a monkeypox antigen and, optionally, a second viral antigen (e.g., a second monkeypox antigen and/or an influenza virus antigen).

In some aspects, the polynucleotides disclosed herein can comprise: a first nucleotide sequence, wherein the first nucleotide sequence encodes a first pathogen protein (e.g., a S1 subunit of the SARS-CoV-2 S protein or a monkeypox protein) and is operably linked to a first promoter (e.g., a hEF1-HTLV promoter); a second nucleotide sequence, wherein the second nucleotide sequence encodes IL-12 p35 and is operably linked to a second promoter (e.g., a CMV promoter); and a third nucleotide sequence, wherein the third nucleotide sequence encodes IL-12 p40 and is operably linked to a second promoter (e.g., a CMV promoter). In some aspects, the first, second, and third nucleotide sequences of the polynucleotide are configured as shown in FIG. 14C (pVac 2).

In some aspects, the polynucleotides disclosed herein can comprise: a first nucleotide sequence, wherein the first nucleotide sequence encodes a first pathogen protein (e.g., a S1 subunit of the SARS-CoV-2 S protein or a monkeypox protein) and is operably linked to a first promoter (e.g., a hEF1-HTLV promoter); a second nucleotide sequence, wherein the second nucleotide sequence encodes a second pathogen protein (e.g., a SARS-CoV-2 M protein or a monkeypox protein) and is operably linked to the first promoter through an IRES sequence; a third nucleotide sequence, wherein the third nucleotide sequence encodes IL-12 p35 and is operably linked to a second promoter (e.g., a CMV promoter); and a fourth nucleotide sequence, wherein the fourth nucleotide sequence encodes IL-12 p340 and is operably linked to a second promoter (e.g., a CMV promoter). In some aspects, the first, second, third, and fourth nucleotide sequences of the polynucleotide are configured as shown in FIG. 14D (pVac 3).

In some aspects, the polynucleotides disclosed herein can comprise: a first nucleotide sequence, wherein the first nucleotide sequence encodes a first pathogen protein (e.g., a SARS-CoV-2 full-length D614G S protein or a monkeypox protein) and is operably linked to a first promoter (e.g., an EF-1α promoter); a second nucleotide sequence, wherein the second nucleotide sequence encodes IL-12 p35 and is operably linked to a second promoter (e.g., a CMV promoter); and a third nucleotide sequence, wherein the third nucleotide sequence encodes IL-12 p40 and is operably linked to a second promoter (e.g., a CMV promoter). In some aspects, the first, second, and third nucleotide sequences of the polynucleotide are configured as shown in FIG. 14F (pVac 5).

In some aspects, the polynucleotides disclosed herein can comprise: a first nucleotide sequence, wherein the first nucleotide sequence encodes a first pathogen protein (e.g., a full-length SARS-CoV-2 D614G S protein or a monkeypox protein) and is operably linked to a first promoter (e.g., an EF-1α promoter); a second nucleotide sequence, wherein the second nucleotide sequence encodes a second pathogen protein (e.g., a SARS-CoV-2 M protein or a monkeypox protein) and is operably linked to the first promoter through an IRES sequence; a third nucleotide sequence, wherein the third nucleotide sequence encodes IL-12 p35 and is operably linked to a second promoter (e.g., a CMV promoter); and a fourth nucleotide sequence, wherein the fourth nucleotide sequence encodes IL-12 p340 and is operably linked to a second promoter (e.g., a CMV promoter). In some aspects, the first, second, third, and fourth nucleotide sequences of the polynucleotide are configured as shown in FIG. 14G (pVac 6).

In some aspects, the polynucleotides disclosed herein can comprise a first nucleotide sequence, wherein the first nucleotide sequence encodes a first pathogen protein (e.g., a S1 subunit of the SARS-CoV-2 S protein or a SARS-CoV-2 full-length D614G S protein or a monkeypox protein) and is operably linked to a first promoter (e.g., an EF-1α promoter). In some aspects, the first nucleotide sequence of the polynucleotide is configured as shown in FIGS. 14B (pVac 1) or 14E (pVac 4).

In some aspects, the polynucleotides disclosed herein can comprise a first nucleotide sequence, wherein the first nucleotide sequence encodes a first pathogen protein (e.g., a SARS-CoV-2 full-length D614G S protein or a monkeypox protein) and is operably linked to a first promoter (e.g., an EF-1α promoter); and a second nucleotide sequence, wherein the second nucleotide sequence encodes a second pathogen (e.g., a SARS-CoV-2 M protein or a monkeypox protein) and is operably linked to a second promoter (e.g., a CMV promoter). In some aspects, the first nucleotide sequence of the polynucleotide is configured as shown in FIG. 14H (pVac 7).

In some aspects, the vector constructs illustrated in any of FIGS. 14A-14H can be modified to replace the S1 subunit of the SARS-CoV-2 S protein or the SARS-CoV-2 full-length D614G S protein (a first nucleotide sequence encoding a first pathogen protein) and/or the SARS-CoV-2 M protein (a second nucleotide sequence encoding a second pathogen protein) with nucleotide sequences encoding any combinations of pathogen antigen or antigenic fragment thereof disclosed herein. In some aspects, the nucleotide sequences encode antigens to a virus, a bacteria or a parasite. In some aspects, the nucleotide sequences encode one or more antigens comprise one or more viral antigens, one or more bacterial antigens, or one or more parasite antigens. In some aspects the vector constructs disclosed herein can be used to express DNA or mRNA.

In some aspects, the polynucleotides encode one or more bacterial antigens selected from a Yersinia pestis antigen, a Mycobacterium tuberculosis antigen, antigenic fragments thereof, or any combinations thereof. In some aspects, the Yersinia pestis antigen is a Yersinia pestis capsular antigen. In some aspects, the Yersinia pestis capsular antigen is F1-Ag or virulence antigen (V-Ag). In some aspects, the Mycobacterium tuberculosis antigen is an Apa antigen, an HP65 antigen, a rAg85A antigen, any antigenic fragments thereof, or any combinations thereof.

In some aspects, the polynucleotides encode one or more viral antigens selected from an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a orthopoxvirus antigen, a monkeypox antigen, a vaccinia antigen, a smallpox antigen, a Epstein bar virus antigen, a nipha virus antigen, a varicella-zoster virus antigen, a Clostridioides difficile antigen, a Streptococcus pneumonia antigen, a Neisseria meningitides antigen, antigenic fragments thereof, or any combinations thereof. In some aspects, the enterovirus antigen is an enterovirus 71 (E71) antigen, a coxsackievirus (Cox) protein antigen, antigenic fragments thereof, or any combinations thereof. In some aspects, the E71 antigen is an E71-VP1 antigen, a glutathione S-transferase (GST)-tagged E71-VP1 antigen, antigenic fragments thereof, or any combinations thereof. In some aspects, the Cox protein antigen is GST-tagged Cox protein antigen. In some aspects, the HSV antigen is an HSV-1 envelope antigen, an HSV-2 envelope antigen, an HSV-2 surface glycoprotein antigen, antigenic fragments thereof, or any combinations thereof. In some aspects, the HSV-2 surface glycoprotein antigen is a gB2 antigen, a gC2 antigen, a gD2 antigen, a gE2 antigen, or antigenic fragments thereof, or any combinations thereof. In some aspects, the HIV antigen is an Env antigen, a Gag antigen, a Nef antigen, a Pol antigen, antigenic fragments thereof, and or combinations thereof. In some aspects, the HPV antigen is a minor capsid protein L2 antigen. In some aspects, the minor capsid protein L2 antigen comprises one or more epitope domains (amino acids 10-36 and/or amino acids 65-89) of minor capsid protein L2. In some aspects, the HCV antigen is a nonstructural 3 (NS3) antigen. In some aspects, the RSV antigen is an F antigen, a G antigen, antigenic fragments thereof, or any combinations thereof. In some aspects, the Dengue virus antigen is an E protein antigen, an E protein domain III (EDIII) antigen, a non-structural protein 1 (NS1) antigen, a DEN-80E antigen, antigenic fragments thereof, or any combinations thereof. In some aspects, the Ebola virus antigen is a spike glycoprotein (GB) antigen, a VP24 antigen, a VP40 antigen, a nucleoprotein (NP) antigen, a VP30 antigen, a VP35 antigen, antigenic fragments thereof, or any combinations thereof. In some aspects, the Zika virus antigen is an envelope domain III antigen, a CKD antigen, antigenic fragments thereof, or any combinations thereof. In some aspects, the Chikungunya virus antigen is an E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), antigenic fragments thereof, or any combinations thereof. In some aspects, the measles virus antigen is a hemagglutinin protein MV-H antigen, a fusion protein MV-F antigen, antigenic fragments thereof, or any combinations thereof. In some aspects, the MERS-CoV antigen is a spike (S) protein antigen, an antigen from the receptor-binding domain of the S protein, an antigen from the membrane fusion domain of the S protein, antigenic fragments thereof, or any combinations thereof. In some aspects, the SARS-CoV antigen is a spike (S) protein antigen, an antigen from the receptor binding domain of the S protein, an antigen from the membrane fusion domain of the S protein, an envelope (E) protein antigen, an M protein antigen, antigenic fragments thereof, or any combinations thereof. In some aspects, the monkeypox antigen is a A35R protein antigen, a H3L protein antigen, a L1R protein antigen, or any combinations thereof.

In some aspects, the polynucleotides encode one or more influenza virus antigens from any influenza virus type or subtype. In some aspects, the one or more influenza virus antigens are selected from the group consisting of: an influenza virus hemagglutinin (HA) antigen, an influenza virus neuraminidase (NA) antigen, an influenza virus matrix-2 (M2) protein antigen, antigenic fragments thereof, and any combination thereof. In some aspects, the one or more influenza virus antigens are derived from influenza virus type A, type B, type C, type D, or any combination thereof. In some aspects, the one or more influenza virus antigens are derived from influenza virus type A. In some aspects, the one or more influenza virus antigens derived from influenza virus type A have (a) a HA subtype selected from H1 through H18 or any combination thereof and (b) a NA subtype selected from N1 through N11 or any combination thereof. In some aspects, the one or more influenza virus antigens derived from influenza virus type A, subtype H1N1; influenza virus type A, subtype H2N2; influenza virus type A, subtype H3N2; influenza virus type A, subtype H5N1; influenza virus type A, subtype H7N7; influenza virus type A, subtype H7N9; influenza virus type A, subtype H9N2; or any combination thereof. In some aspects, the one or more influenza virus antigens are derived from influenza virus type A, subtype H1N1; influenza virus type A, subtype H3N2; or the combination thereof. In some aspects, the one or more influenza virus antigens are derived from influenza virus type B. In some aspects, the nucleotide sequences encode one or more SARS-CoV-2 antigens or antigenic fragments thereof disclosed herein and one or more influenza virus antigens or antigenic fragments thereof disclosed herein. In some aspects, the nucleotide sequences encode at least two SARS-CoV-2 antigens or antigenic fragments thereof disclosed herein. In some aspects, the at least two SARS-CoV-2 antigens or antigenic fragments thereof are derived from different strains of SARS-CoV-2. In some aspects, the at least two SARS-CoV-2 antigens or antigenic fragments thereof are different variants of the same SARS-CoV-2 antigen or antigenic fragment thereof, wherein the different variants of the same SARS-CoV-2 antigen or antigenic fragment thereof are derived from different strains of SARS-CoV-2. In some aspects, the nucleotide sequences encode one or more monkeypox antigens or antigenic fragments thereof disclosed herein and one or more influenza virus antigens or antigenic fragments thereof disclosed herein. In some aspects, the nucleotide sequences encode at least two monkeypox antigens or antigenic fragments thereof disclosed herein. In some aspects, the at least two monkeypox antigens or antigenic fragments thereof are derived from different strains of monkeypox. In some aspects, the at least two monkeypox antigens or antigenic fragments thereof are different variants of the same monkeypox antigen or antigenic fragment thereof, wherein the different variants of the same monkeypox antigen or antigenic fragment thereof are derived from different strains of monkeypox.

In some aspects, the polynucleotides encode one or more parasite antigens, wherein the one or more parasite antigens comprise one or more protozoan antigens. In some aspects, the polynucleotides encode one or more parasite antigens selected from Toxoplasma gondii antigen, a Plasmodium falciparum antigen, antigenic fragments thereof, or any combinations thereof. In some aspects, the Toxoplasma gondii antigen is antigen MIC8. In some aspects, the Plasmodium falciparum antigen is a SERA5 polypeptide antigen, a circumsporozite protein antigen, antigenic fragments thereof, or any combinations thereof. In some aspects, the polynucleotides encode one or more parasite antigens, wherein the one or more parasite antigens comprise one or more parasitic or pathogenic fungus antigens.

In some aspects, the one or more parasitic or pathogenic fungus antigens are selected from the group consisting of a Candida spp. antigen (e.g., a Candida albicans antigen, a Candida glabrata antigen, a Candida parapsilosis antigen, a Candida tropicalis antigen, a Candida lusitaniae antigen, a Candida krusei antigen), a Pneumocystis spp. antigen, a Malassezia spp. antigen (e.g., a Malassezia furfur antigen), an Aspergillus fumigatus antigen, a Cryptococcus spp. antigen (e.g., a Cryptococcus neoformans antigen, a Cryptococcus gattii antigen), a Histoplasma capsulatum antigen, a Blastomyces dermatitidis antigen, a Paracoccidioides spp. antigen (e.g., a Paracoccidioides brasiliensis antigen, a Paracoccidioides lutzii antigen), a Coccidioides spp. antigen (e.g., a Coccidioides immitis antigen, a Coccidioides posadasii antigen), a Penicillium marneffei antigen, a Sporothrix schenckii antigen, a Trichosporon asahii antigen, a Fusarium spp. antigen (e.g., a Fusarium solanum antigen, a Fusarium oxysporum antigen), a Nectria spp. antigen, a Pseudoallescheria boydii antigen, a Cladophialphora bantianum antigen, a Ramichloridium spp. antigen, a Dactylaria gallopava antigen, an Exophiala spp. antigen (e.g., an Exophiala jeanselmei antigen, an Exophiala dermatitidis antigen), a Curvularia spp. antigen, a Bipolaris spp. antigen, an Alternaria spp. antigen, a Lacazia loboi antigen, a Conidiobolus spp. antigen (e.g., a Conidiobolus coronatus antigen, a Conidiobolus incongruus antigen), and any combination thereof.

In some aspects, the first pathogen protein is a SARS-CoV-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the first SARS-CoV-2 protein comprises polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the first SARS-CoV-2 protein comprises the amino acid sequence of SEQ ID NO: 2 or SEQ ID NO: 4.

In some aspects, the second pathogen protein is a SARS-CoV-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 2 or SEQ ID NO: 4. In some aspects, the second SARS-CoV-2 protein comprises the amino acid sequence of SEQ ID NO: 2 or SEQ ID NO: 4.

In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the first SARS-CoV-2 protein comprises the amino acid sequence of SEQ ID NO: 2 or SEQ ID NO: 4, wherein the polypeptide comprises one or more mutations (i.e., one or more substitutions, deletions, insertions, or any combination thereof).

In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 750, at least 1,000, or at least 1,250 contiguous amino acids of SEQ ID NO: 2 or SEQ ID NO: 4, wherein the contiguous amino acids of SEQ ID NO: 2 or SEQ ID NO: 4 comprise one or more mutations (i.e., one or more substitutions, deletions, insertions, or any combination thereof). In some aspects, the second SARS-CoV-2 protein comprises the amino acid sequence of SEQ ID NO: 2 or SEQ ID NO: 4, wherein the polypeptide comprises one or more mutations (i.e., one or more substitutions, deletions, insertions, or any combination thereof).

In some aspects, the first SARS-CoV-2 protein is the receptor binding domain (RBD) of the SARS-Cov-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the first SARS-CoV-2 protein comprises polypeptide having at least 70%, at least 75%, at least 80%, at least 85%, at least 90%, at least 95%, or at least 99% sequence identity to the amino acid sequence of SEQ ID NO: 6. In some aspects, the first SARS-CoV-2 protein comprises the amino acid sequence of SEQ ID NO: 6.

In some aspects, the second SARS-CoV-2 protein is the receptor binding domain (RBD) of the SARS-Cov-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 6. In some aspects, the second SARS-CoV-2 protein comprises the amino acid sequence of SEQ ID NO: 6.

In some aspects, the first SARS-CoV-2 protein is the receptor binding domain (RBD) of the SARS-Cov-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the first SARS-CoV-2 protein comprises the amino acid sequence of SEQ ID NO: 6, wherein the polypeptide comprises one or more mutations (i.e., one or more substitutions, deletions, insertions, or any combination thereof).

In some aspects, the second SARS-CoV-2 protein is the receptor binding domain (RBD) of the SARS-Cov-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous amino acids of SEQ ID NO: 6, wherein the contiguous amino acids of SEQ ID NO: 6 comprise one or more mutations (i.e., one or more substitutions, deletions, insertions, or any combination thereof). In some aspects, the second SARS-CoV-2 protein comprises the amino acid sequence of SEQ ID NO: 6, wherein the polypeptide comprises one or more mutations (i.e., one or more substitutions, deletions, insertions, or any combination thereof).

In some aspects, the first SARS-CoV-2 protein is the S1 subunit of the SARS-Cov-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the first SARS-CoV-2 protein comprises polypeptide having at least 70%, at least 75%, at least 80%, at least 85%, at least 90%, at least 95%, or at least 99% sequence identity to the amino acid sequence of SEQ ID NO: 40. In some aspects, the first SARS-CoV-2 protein comprises the amino acid sequence of SEQ ID NO: 40.

In some aspects, the second SARS-CoV-2 protein is the S1 subunit of the SARS-Cov-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 40. In some aspects, the second SARS-CoV-2 protein comprises the amino acid sequence of SEQ ID NO: 40.

In some aspects, the first SARS-CoV-2 protein is the S1 subunit of the SARS-Cov-2 S protein or an antigenic fragment thereof. In some aspects, the first SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the first SARS-CoV-2 protein comprises the amino acid sequence of SEQ ID NO: 40, wherein the polypeptide comprises one or more mutations (i.e., one or more substitutions, deletions, insertions, or any combination thereof).

In some aspects, the second SARS-CoV-2 protein is the S1 subunit of the SARS-Cov-2 S protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 550, at least 600, at least 650, or at least 660 contiguous amino acids of SEQ ID NO: 40, wherein the contiguous amino acids of SEQ ID NO: 40 comprise one or more mutations (i.e., one or more substitutions, deletions, insertions, or any combination thereof). In some aspects, the second SARS-CoV-2 protein comprises the amino acid sequence of SEQ ID NO: 40, wherein the polypeptide comprises one or more mutations (i.e., one or more substitutions, deletions, insertions, or any combination thereof).

In some aspects, the one or more mutations in the SARS-CoV-2 full-length S protein, the RBD of the SARS-Cov-2 S protein, the S1 subunit of the SARS-CoV-2 S protein, or antigenic fragments thereof comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety.

In some aspects, the one or more mutations in the SARS-CoV-2 full-length S protein, the RBD of the SARS-Cov-2 S protein, the S1 subunit of the SARS-CoV-2 S protein, or antigenic fragments thereof are selected from: AM1-S13, S12P, S13I, L5F, L18F, T19R, T20N, P26S, Q52R, A67V, AH69-V70, G75V, T76I, D80A, T95I, R102I, AD119-F120, C136Y, D138Y, AF140, AL141-Y144, AY144, Y144S, Y145N, AH146, N148S, K150R, K150E, K150T, K150Q, S151P, W152C, E154K, AE156-F157, F157L, F157A, R158G, R190S, AI210, D215G, A222V, AL241-S243, AL242-L244, AA243-L244, AR246-G252, R246I, 11-amino acid residue insertion between Y248 and L249, D253G, D253N, R346K, V367F, E406W, K417E, K417V, K417N, K417T, N439K, K444A, K444R, K444N, K444Q, V445A, V445E, G447A, N450D, L452R, L452Q, Y453F, L455F, N460I, S477G, S477N, S477R, T478I, T478K, V483I, E484K, E484Q, G485R, F486A, F486V, F486L, N487A, F490S, Q493E, Q493K, S494P, N501Y, A570D, Q613H, D614G, H655Y, Q677H, A678-679, A681-682, A681-684, A682-685, P681H, P681R, R682S, R682A, R682Q, R683S, R683G, R683Q, R685G, R685Q, I692V, A701V, T716I, F817P, T859N, F888L, A892P, A899P, A942P, D950N, S982A, K986P, V987P, T1027I, Q1071H, D1118H, V1176F, M1229I, AC1253-T1273, AC1254-T1273, AK1255-T1273, D1257A, E1258A, D1259A, D1260A, E1262A, K1269A, H1271K, T1273A, or any combination thereof, wherein the amino acid locations correspond to SEQ ID NO: 2 or SEQ ID NO: 4.

In some aspects, the one or more mutations in the polypeptide(s) encoded by the antigen nucleic acid (e.g., first antigen nucleic acid) and/or the at least one additional antigen nucleic acid (e.g., second antigen nucleic acid) are selected from: A67V, ΔH69-V70, T95I, G142D, ΔV143, ΔY144, Y145D, N211I, Δ212I, 214EPEins, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, and N969K, L981.

In some aspects, the one or more mutations in the polypeptide(s) encoded by the antigen nucleic acid (e.g., first antigen nucleic acid) and/or the at least one additional antigen nucleic acid (e.g., second antigen nucleic acid) are selected from: T19I, LPPA24-27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, and N969K.

In some aspects, the one or more mutations comprise one or more mutations in the N-terminal signal peptide, which corresponds to amino acids 1-13 of SEQ ID NO: 2 or SEQ ID NO: 4. In some aspects, the one or more mutations in the N-terminal signal peptide is AM1-S13, wherein the amino acid locations correspond to SEQ ID NO: 2 or SEQ ID NO: 4.

In some aspects, the one or mutations comprise one or more mutations in the C-terminus of the full-length SARS-CoV-2 S protein. In some aspects, the one or more mutations in the C-terminus of the full-length SARS-CoV-2 S protein comprise one or more mutations in the C-terminal endoplasmic reticulum (ER) retention peptide, which corresponds to amino acids 1254-1273 of SEQ ID NO: 2 or SEQ ID NO: 4. In some aspects, the one or more mutations in the C-terminal ER retention peptide are selected from D1257A, E1258A, D1259A, D1260A, E1262A, K1269A, H1271K, T1273A, or any combination thereof, wherein the amino acid locations correspond to SEQ ID NO: 2 or SEQ ID NO: 4. In some aspects, the one or more mutations in the C-terminal ER retention peptide comprise D1257A+E1258A+D1259A+D1260A+E1262A (i.e., a D/E to A mutant), wherein the amino acid locations correspond to SEQ ID NO: 2 or SEQ ID NO: 4. In some aspects, the one or mutations in the C-terminal ER retention peptide is ΔC1253-T1273, AC1254-T1273, or AK1255-T1273.

In some aspects, the one or more mutations comprise K986P+V987P (i.e., a S-2P mutation), wherein the amino acid locations correspond to SEQ ID NO: 2 or SEQ ID NO: 4. In some aspects, the one or more mutations comprise F817P+A892P+A899P+A942P (i.e., a hexa-proline S mutation), wherein the amino acid locations correspond to SEQ ID NO: 2 or SEQ ID NO: 4. In some aspects, the one or more mutations comprise one or more mutations in the 681-PRRAR/SVA-688 S1/S2 furin cleavage site, wherein the amino acid locations correspond to SEQ ID NO: 2 or SEQ ID NO: 4. In some aspects, the one or more mutations in the 681-PRRAR/SVA-688 S1/S2 furin cleavage site are: (a) R682S+R683S (i.e., a SSAR mutation), (b) A681-684 (i.e., a APRRA mutation), (c) A678-679+A681-682, (d) R682A+R683G+R685G (i.e., a 682-AGAG-685 mutation), (e) R682Q+R683Q+R685Q, (f) R682S+R685G, or (g) A682-685 (i.e., a ARRAR mutation), wherein the amino acid locations correspond to SEQ ID NO: 2 or SEQ ID NO: 4.

In some aspects, the one or more mutations comprise: (a) F817P+A892P+A899P+A942P (i.e., a hexa-proline S mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation) and (b) K986P+V987P (i.e., a S-2P mutation), wherein the amino acid locations correspond to SEQ ID NO: 2 or SEQ ID NO: 4. In some aspects, the one or more mutations comprise: (a) R682A+R683G+R685G (i.e., a 682-AGAG-685 mutation), (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682Q+R683Q+R685Q and (b) K986P+V987P (i.e., a S-2P mutation). In some aspects, the one or more mutations comprise: (a) R682Q+R683Q+R685Q, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4. In some aspects, the one or more mutations comprise: (a) R682S+R685G and (b) K986P+V987P (i.e., a S-2P mutation), wherein the amino acid locations correspond to SEQ ID NO: 2 or SEQ ID NO: 4. In some aspects, the one or more mutations comprise: (a) R682S+R685G, (b) K986P+V987P (i.e., a S-2P mutation), and (c) F817P+A892P+A899P+A942P (i.e., a hexa-proline S mutation), wherein the amino acid locations correspond to SEQ ID NO: 2 or SEQ ID NO: 4.

In some aspects, the second SARS-CoV-2 protein is a SARS-CoV-2 M protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20. In some aspects, the second SARS-CoV-2 protein comprises the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20.

In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the contiguous amino acids of SEQ ID NO: 8, SEQ ID NO: 10, SEQ ID NO: 12, SEQ ID NO: 14, SEQ ID NO: 16, SEQ ID NO: 18, or SEQ ID NO: 20 comprise one or more mutations selected from A2S, F28L, I48V, V70L, I82T, M84T, or any combination thereof, wherein the amino acid locations correspond to SEQ ID NO: 8. In some aspects, the second SARS-CoV-2 protein comprises the amino acid sequence of SEQ ID NO: 8, SEQ ID NO: 10, SEQ ID NO: 12, SEQ ID NO: 14, SEQ ID NO: 16, SEQ ID NO: 18, or SEQ ID NO: 20, wherein the polypeptide comprises one or more mutations selected from A2S, F28L, I48V, V70L, I82T, M84T, or any combination thereof, wherein the amino acid locations correspond to SEQ ID NO: 8.

In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, or at least 220 contiguous amino acids of SEQ ID NO: 2, wherein the contiguous amino acids of SEQ ID NO: 2 comprise one or more mutations selected from T19I, LPPA24-27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K.

In some aspects, the second SARS-CoV-2 protein is a SARS-CoV-2 E protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 55, at least 60, at least 65, at least 70, or at least 75 contiguous amino acids of SEQ ID NO: 22, SEQ ID NO: 24, or SEQ ID NO: 26. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 22, SEQ ID NO: 24, or SEQ ID NO: 26. In some aspects, the second SARS-CoV-2 protein comprises the amino acid sequence of SEQ ID NO: 22, SEQ ID NO: 24, or SEQ ID NO: 26.

In some aspects, the second SARS-CoV-2 protein is a SARS-CoV-2 N protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 28. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 28. In some aspects, the second SARS-CoV-2 protein comprises the amino acid sequence of SEQ ID NO: 28.

In some aspects, the second SARS-CoV-2 protein is a SARS-CoV-2 N protein or an antigenic fragment thereof. In some aspects, the second SARS-CoV-2 protein comprises at least 8, at least 10, at least 15, at least 20, at least 25, at least 30, at least 35, at least 40, at least 45, at least 50, at least 60, at least 70, at least 80, at least 90, at least 100, at least 150, at least 200, at least 250, at least 300, at least 350, or at least 400 contiguous amino acids of SEQ ID NO: 123. In some aspects, the second SARS-CoV-2 protein comprises polypeptide having at least 70%, at least 75%, at least 80%, at least 85%, at least 90%, at least 95%, or at least 99% sequence identity to the amino acid sequence of SEQ ID NO: 123. In some aspects, the second SARS-CoV-2 protein comprises the amino acid sequence of SEQ ID NO: 123.

In some aspects, promoter 1 is selected from the group consisting of: a CMV promoter, an RSV promoter, a Mo-MuLV LTR promoter, a mammalian EF 1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF 1 promoter is a hEF1-HTLV promoter. In some aspects, promoter 1 is selected from the group consisting of: a CMV promoter, an RSV promoter, a Mo-MuLV LTR promoter, a mammalian EF1 promoter, a CK18 promoter, a CK19 promoter, and any combination thereof.

In some aspects, promoter 2 is selected from the group consisting of: a CMV promoter, an RSV promoter, a Mo-MuLV LTR promoter, a mammalian EF 1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF 1 promoter is a hEF1-HTLV promoter. In some aspects, promoter 2 is selected from the group consisting of: a CMV promoter, an RSV promoter, a Mo-MuLV LTR promoter, a mammalian EF1 promoter, a CK18 promoter, a CK19 promoter, and any combination thereof.

In some aspects, promoter X is selected from the group consisting of: a CMV promoter, an RSV promoter, a Mo-MuLV LTR promoter, a mammalian EF 1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CAG promoter, and any combination thereof. In some aspects, the mammalian EF 1 promoter is a hEF1-HTLV promoter. In some aspects, promoter X is selected from the group consisting of: a CMV promoter, an RSV promoter, a Mo-MuLV LTR promoter, a mammalian EF1 promoter, a CK18 promoter, a CK19 promoter, and any combination thereof.

In some aspects, promoter Y is selected from the group consisting of: a CMV promoter, an RSV promoter, a Mo-MuLV LTR promoter, a mammalian EF 1 promoter, a CK18 promoter, a CK19 promoter, an SV40 promoter, a murine U6 promoter, a skeletal α-actin promoter, a R-actin promoter, a murine PGK1 promoter, a human PGK1 promoter, a CAG promoter, and any combination thereof. In some aspects, the mammalian EF 1 promoter is a hEF1-HTLV promoter. In some aspects, promoter Y is selected from the group consisting of: a CMV promoter, an RSV promoter, a Mo-MuLV LTR promoter, a mammalian EF1 promoter, a CK18 promoter, a CK19 promoter, and any combination thereof.

In some aspects, promoter Z is selected from the group consisting of: a CMV promoter, an RSV promoter, a Mo-MuLV LTR promoter, a mammalian EF 1 promoter, a CK18 promoter, a CK19 promoter, an SV40 promoter, a murine U6 promoter, a skeletal α-actin promoter, a j-actin promoter, a murine PGK1 promoter, a human PGK1 promoter, a CAG promoter, and any combination thereof. In some aspects, the mammalian EF 1 promoter is a hEF1-HTLV promoter. In some aspects, promoter Z is selected from the group consisting of: a CMV promoter, an RSV promoter, a Mo-MuLV LTR promoter, a mammalian EF1 promoter, a CK18 promoter, a CK19 promoter, and any combination thereof. In some aspects, the mammalian EF 1 promoter is a hEF1-HTLV promoter.

In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

### V. Vectors and Host Cells

Also provided herein are vectors or constructs comprising any polynucleotide described or exemplified herein, wherein the vector is a DNA plasmid vector, a multicistronic mRNA vector, a viral vector, a bacterial vector, a cosmid, or an artificial chromosome. In some aspects, the vector is selected from an AAV vector, adenoviral vector, retroviral vector, poxvirus vector, baculovirus vector, herpes viral vector, or combinations thereof.

In some aspects, the vectors are DNA plasmid vectors and multicistronic mRNA vectors comprising any polynucleotide described or exemplified herein.

Also provided herein are recombinant host cells comprising any polynucleotide, vector, DNA plasmid vector, or vaccine described herein. Host cells include prokaryotic cells, lower eukaryotic cells such as yeast, other eukaryotic cells such as insect cells, and higher eukaryotic cells such as mammalian cells. Mammalian host cells include, but are not limited to, CHO, VERO, BHK, Hela, MDCK, HEK 293, NIH 3T3, W138, BT483, Hs578T, HTB2, BT20 and T47D, NSO (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10, HBK, NSO, HT1080 and HsS78Bst cells. In some aspects, the recombinant host cells arc prepared by introducing the vectors, polynucleotides, or vaccines described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook. et al. (Molecular Cloning. A Laboratory Manual. 2nd. ed. Cold Spring Harbor Laboratory. Cold Spring Harbor Laboratory Press. Cold Spring Harbor. NY (1989).

### VI. Delivery Components

In some aspects, the disclosed compositions, pharmaceutical compositions, vaccines, vectors, multicistronic mRNA vectors, or DNA plasmid vectors further comprise a delivery component. In some aspects, the delivery component is a non-viral delivery component or system based on “naked DNA” or formulated plasmid DNA. In some aspects, the delivery component or system can be used to deliver mRNA. In some aspects, the non-viral delivery component is a cationic polymer. In some aspects, the cationic polymer is a synthetic functionalized polymer, a lipid, a lipopolymer, or a chemical derivative thereof.

Non-viral gene delivery components or systems, based on “naked DNA” or formulated plasmid DNA, have potential benefits over viral vectors due to simplicity of use and lack of inciting a specific immune response. A number of synthetic gene delivery systems have been described to overcome the limitations of naked DNA, including cationic lipids, peptides, and polymers. Similarly, non-viral delivery systems can be used for delivery of mRNA.

Polymers have emerged as a viable alternative to current systems because their excellent molecular flexibility allows for complex modifications and incorporation of novel chemistries. Cationic polymers, such as poly(L-lysine) (PLL) and poly(L-arginine) (PLA), polyethyleneimine (PEI) have been widely studied as gene delivery candidates due to their ability to condense DNA, and promote DNA stability and transmembrane delivery. PEI efficiently condenses DNA into small narrowly distributed positively charged spherical complexes and can transfect cells in vitro and in vivo. PEI is similar to other cationic polymers in that the transfection activity of PEI increases with increasing polymer/DNA ratios. A distinct advantage of PEI over PLL is its endosomolytic activity which enables PEI to yield high transfection efficiency. Commercial branched PEI is composed of 25% primary amines, 50% secondary amines and 25% tertiary amines. The overall protonation level of PEI doubles from pH 7 to pH 5, which means in the endosome PEI becomes heavily protonated. Protonation of PEI triggers chloride influx across the endosomal membrane, and water follows to counter the high ion concentration inside the endosome, which eventually leads to endosomal disruption from osmotic swelling and release of the entrapped DNA. Because of its intrinsic endosomolytic activity, PEI generally does not require the addition of an endosomolytic agent for transfection. Due to these advantages PEI has been increasingly utilized in polymer functionalization strategies to create safer and more efficient delivery systems.

In some aspects, the delivery component is a cationic polymer. In some aspects, the cationic polymer is a synthetic functionalized polymer, a lipid, a lipopolymer, or a chemical derivative thereof. In some aspects, the cationic polymer is present in an amount sufficient to produce a ratio of amine nitrogen in the cationic polymer to phosphate in the DNA plasmid vector or multicistronic mRNA vector from about 0.1:1 to about 100:1. In some aspects, the ratio of amine nitrogen in the cationic polymer to phosphate in the DNA plasmid vector or multicistronic mRNA vector is from about 0.1:1 to about 10:1. In some aspects, the composition, pharmaceutical composition, or vaccine comprises about 0.5 mg/ml to about 5.0 mg/ml nucleic acid complexed with the cationic polymer. In some aspects, the delivery component is a poloxamer or a derivative thereof. In some aspects, the poloxamer or derivative thereof is present in a solution with the polynucleotide, multicistronic mRNA vector, or DNA plasmid vector from about 0.1% to about 5% or about 0.5%-about 5%. In some aspects, the delivery component is a 3-amino ester. In some aspects, the polymer is present in a solution with the polynucleotide, multicistronic mRNA vector, or DNA plasmid vector from about 0.1% to about 5% or about 0.5%-about 5%. In some aspects, the delivery component is a poly-inosinic-polycytidylic acid. In some aspects, the poly-inosinic-polycytidylic acid is present in a solution with the polynucleotide, multicistronic mRNA vector, or DNA plasmid vector from about 0.1% to about 5% or about 0.5%-about 5%.

### VI.1 Biodegradable Cross-linked Cationic Multi-block Copolymers

In some aspects, the delivery component is a biodegradable cross-linked cationic multi-block copolymer, for example any biodegradable cross-linked cationic multi-block copolymer disclosed in U.S. Pat. No. 8,445,017. In some aspects, the biodegradable cross-linked cationic multi-block copolymer is a biodegradable cross-linked cationic multi-block copolymer of linear poly(alkylenimine) (LPAI) and a hydrophilic linker, wherein said LPAI blocks are crossed linked together by said hydrophilic linker with biodegradable ester, amide, disulfide, or phosphate linkages bonds. In some aspects, the linear poly(alkylenimine) (LPAI) is a member selected from the group consisting of polyethyleneimine, polypropylenimine, aminoglycoside-polyamine, dideoxy-diamino-.beta.-cyclodextrin, spermine and spermidine. In some aspects, the linear poly(alkylenimine) (LPAI) is linear poly(ethylenimine) (LPEI).

In some aspects, the cross-linked cationic multi-block copolymer is linked by the biodegradable linkers to other moieties such as, for example, fluorescent markers, lipids anchors or their derivatives, i.e., cholesterol, fatty acids or their derivatives. In some aspects, the molecular weight of the linear PEI used in this disclosure is within the range of 1,000 to 25,000 Dalton. In some aspects, the linear PEI blocks are preferably linked to one another via a diamide linkage utilizing a biodegradable disulfidediacid-derived linker, i.e., dithiodipropionate derivatives. In some aspects, the molar ratio of the linker to the PEI is within a range of 1/1 to 5/1; the molar ratio of the lipid anchors to PEI is from 0/1 to 3/1. In some aspects, the polymer is formulated as a polyammonium salt, preferably with a chloride counterion. Since the toxicity of PEI increases with an increase in its molecular weight, the use of lower molecular weight PEIs as blocks in the polymer provides an improved gene carrier for use as a general reagent for transfection of mammalian cells, and for the in vivo application of gene therapy.

In some aspects, the biodegradable, cross-linked cationic multi-block copolymer comprises low molecular weight linear PEI blocks and a dithioacid moiety, i.e., dithiodipropionic acid, as biodegradable linkers. The biodegradable, cross-linked cationic multi-block copolymers are synthesized by cross-linking low molecular weight linear PEI units via a biodegradable disulfide linkage. These biodegradable cross-linked cationic multi-block copolymers are water soluble and transfectionally superior (68-70 fold higher activity) to single block polymers. See U.S. Pat. No. 8,445,017.

In some aspects, the linker to the polymer molecular weight ratio is <0.2 which minimizes the dilution of polyamine polymer backbone. In some aspects, the chemical bond between the linker and the polymer blocks is a disulfide bond which can be biodegraded more easily as compared to amide bonds. Other biodegradable bonds can also be used in the present disclosure includes: phosphoesters, hydrazone, cis-asotinyl, urethane and poly(ethyl). Since any linker reacts in stepwise fashion, it can link either different blocks or the different areas of the same block (loop formation). The latter will favor the formation of a lightly cross-linked material with poor solubility due to multiple looping. The process disclosed in U.S. Pat. No. 8,445,017 solves this problem by incorporating partial and reversible blocking/protection of nitrogen atoms in the LPEI blocks. Such LPEI functionalization also increases polymer solubility, facilitating the linking of LPEI blocks. This process also allows for convenient incorporation of pendant auxiliary ligands (for example, lipids, or fluorescent markers) onto a cationic polymer.

In some aspects, the cationic block copolymers are represented by the following formula: (CP)ₓL_{y}Y_{z} wherein CP represents a cationic polymer containing at least one secondary amine group, said CP polymer has a number averaged molecular weight within the range of 1,000 to 25,000 Dalton; Y represents a bifunctional biodegradable linker containing ester, amide, disulfide, or phosphate linkages; L represents a ligand; x is an integer in the range from 1 to 20; y is an integer from 0 to 100; and z is an integer in the range from 0 to 40. In some aspects, the cationic polymer comprises linear polyethyleneimine (LPEI). In some aspects, the LPEI is BD15-12, which has the following formula:

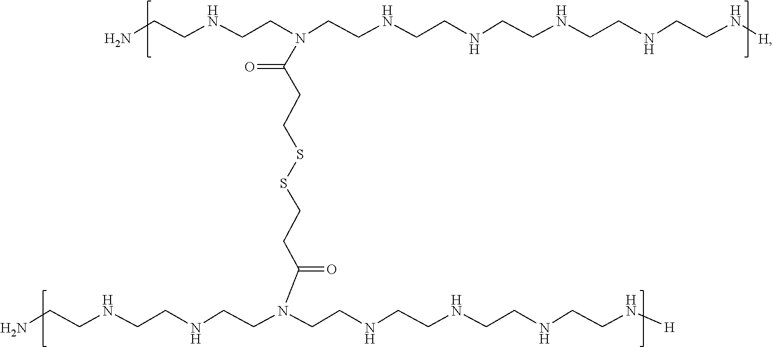

wherein the PEIs are approximately 15,000 Da, and wherein there is an average of 12 crosslinkers per PEI. In some aspects, the LPEI is Omnifect, which has the following formula:

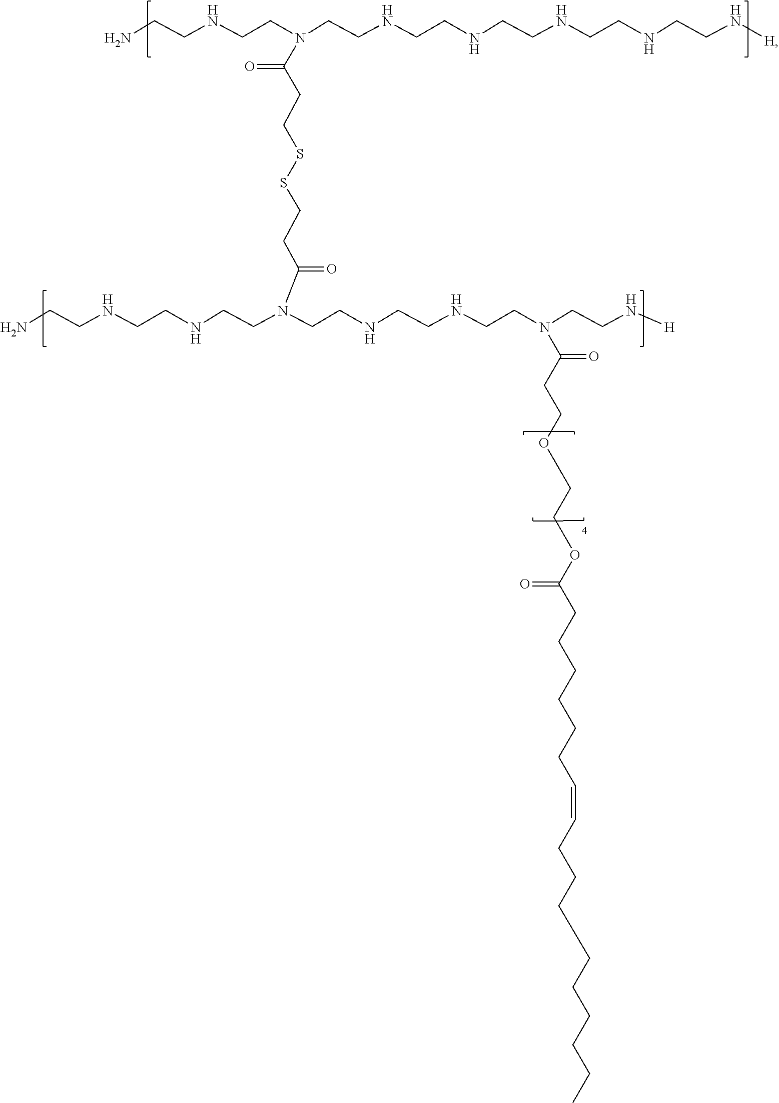

wherein the PEIs are approximately 3,600 Da, wherein there is an average of 3 crosslinkers per PEI, and wherein there is an average of 1 PEG-lipid per PEI. In some aspects, the bifunctional biodegradable linker is hydrophilic and comprises a biodegradable linkage comprising a disulfide bond. In some aspects, the bifunctional biodegradable linker is a dithiodipropionyl linker.

In some aspects, the biodegradable cross-linked cationic multi-block copolymer comprises LPEI and a dithiodipropionyl linker for cross-linking the multi-block copolymer, wherein the LPEI has an average molecular weight of 1,000 to 25,000 Dalton. In some aspects, the biodegradable cross-linked cationic multi-block copolymer is covalently linked to at least one ligand.

In some aspects, the biodegradable, cross-linked, cationic, multi-block copolymers of LPEI and lipopolymers of the present disclosure have amine groups that are electrostatically attracted to polyanionic compounds such as nucleic acids. In some aspects, the cationic copolymer condenses DNA and forms compact structures. In addition, low toxicity of monomeric degradation products after delivery of bioactive materials provides for gene carriers with reduced cytotoxicity and increased transfection efficiency.

In some aspects, the biodegradable cross-linked cationic multi-block copolymers are conjugated with tracers (for example, fluorescent markers) or ligands either directly or via spacer molecules. In some aspects, only a small portion of the available amino groups is coupled to the ligand. The ligands conjugated to the polymers are targeting ligands that direct the polymers-nucleic acid complex to bind to specific target cells and penetrate into such cells. The targeting ligands can also be an intracellular targeting element, enabling the transfer of the nucleic acid/drug to be guided towards certain favored cellular compartments (mitochondria, nucleus, and the like). The targeting ligands conjugated to the polymers direct the polymers-nucleic acid complex to bind to specific target cells and penetrate into such cells (e.g., epithelial cells, endothelial cells, hematopoietic cells, and the like). The target ligands can also be an intracellular targeting element, enabling the transfer of the nucleic acid/drug to be guided towards certain favored cellular compartments (mitochondria, nucleus, and the like).

In some aspects, the targeting ligand is a polypeptide, folate, and an antigen. In some aspects, the polypeptide is a glycoprotein (e.g., transferrin or asialoorosomucoid (ASOR)), an antibody, an antibody fragment, a cell receptor, a cytokine receptor, or a growth factor receptor (e.g., epidermal growth factor receptor). In some aspects, the antigen is a viral antigen, a bacterial antigen, or a parasite antigen. In some aspects, the ligand is a fusogenic agent (e.g., polymixin B and hemaglutinin HA2), a lysosomotrophic agent, or a nucleus localization signal (NLS) (e.g., T-antigen, and the like). In some aspects, the ligand is a sugar moiety coupled to an amino group. In some aspects, the sugar moiety is a mono- or oligo-saccharide, such as galactose, glucose, fucose, fructose, lactose, sucrose, mannose, cellobiose, nytrose, triose, dextrose, trehalose, maltose, galactosamine, glucosamine, galacturonic acid, glucuronic acid, and gluconic acid.

In some aspects, the biodegradable cross-linked cationic multi-block copolymer is covalently linked to polyethylene glycol (PEG) of molecular weight ranging from 500 to 20,000 Dalton. In some aspects, the biodegradable cross-linked cationic multi-block copolymer is covalently linked to a fatty acyl chain selected from the group consisting of: oleic acid, palmitic acid, and stearic acid. In some aspects, the biodegradable cross-linked cationic multi-block copolymer comprises at least one amine group that is electrostatically attracted to a polyanionic compound. In some aspects, the polyanionic compound is a nucleic acid, wherein the biodegradable cross-linked cationic multi-block copolymer condenses the nucleic acid to form a compact structure.

In some aspects, the biodegradable cross-linked cationic multi-block copolymer has the following formula.

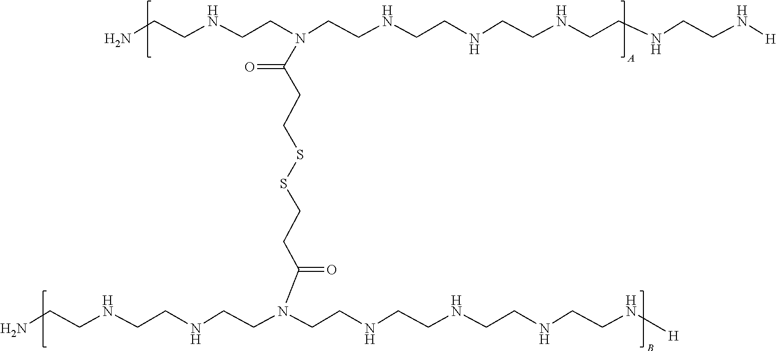

wherein A and B are such that the molecular weight of the individual linear polyethylenimine chains are from 5,000 to 25,000 Dalton; the intermolecular crosslinks connect approximately 5-10% of amines; the biodegradable crosslinks are dithiodipropionyl (each half composed of 3 carbon atoms) and can be from 1-10 carbon atoms. In some aspects, the biodegradable cross-linked cationic polymer comprises 10,000 to 15,000 Dalton linear PEI covalently connected with a dithiopropionyl linkage (see, e.g., U.S. Pat. No. 8,445,017). In some aspects, the biodegradable cross-linked cationic polymer comprises 10,000 to 15,000 Dalton linear PEI covalently connected with a dithiopropionyl linkage (see, e.g., U.S. Pat. No. 8,445,017) and is further conjugated to polyethyleneglycol (PEG) of molecular weight ranging from 500 to 20,000 Dalton. In some aspects, the biodegradable cross-linked cationic polymer comprises 10,000 to 15,000 Dalton linear PEI covalently connected with a dithiopropionyl linkage (see, e.g., U.S. Pat. No. 8,445,017) and is further conjugated to polyethyleneglycol (PEG) of molecular weight ranging from 500 to 20,000 Dalton. In some aspects, the biodegradable cross-linked cationic polymer comprises 15,000 to 20,000 Dalton linear PEI covalently connected with a dithiopropionyl linkage (see, e.g., U.S. Pat. No. 8,445,017). In some aspects, the biodegradable cross-linked cationic polymer comprises 15,000 to 20,000 Dalton linear PEI covalently connected with a dithiopropionyl linkage (see, e.g., U.S. Pat. No. 8,445,017) and is further conjugated to polyethyleneglycol (PEG) of molecular weight ranging from 500 to 20,000 Dalton.

In some aspects, the biodegradable cross-linked cationic multi-block copolymer is present in an amount sufficient to produce a ratio of amine nitrogen in the biodegradable cross-linked cationic multi-block copolymer to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the biodegradable cross-linked cationic multi-block copolymer to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the biodegradable cross-linked cationic multi-block copolymer to phosphate in the polynucleotide, multicistronic mRNA vector, or DNA plasmid vector is from about 1:10 to about 10:1.

In some aspects, the composition, pharmaceutical composition, or vaccine comprises about 0.1 mg/ml to about 10 mg/ml (e.g., about 0.1 mg/ml to about 5 mg/ml; about 0.5 mg/ml to about 10 mg/ml; or about 0.5 mg/ml to about 5 mg/ml) nucleic acid complexed with the biodegradable cross-linked cationic multi-block copolymer. In some aspects, the composition, pharmaceutical composition, or vaccine comprises about 1 mg/ml to about 10 mg/ml (e.g., about 1 mg/ml to about 6 mg/ml; about 2 mg/ml to about 6 mg/ml; about 5 mg/ml to about 10 mg/ml; or about 6 mg/ml to about 10 mg/ml) nucleic acid complexed with the biodegradable cross-linked cationic multi-block copolymer.

### VI.2 Cationic Lipopolymers Comprising a PEI Backbone

In some aspects, the delivery component is a cationic lipopolymer comprising a PEI backbone covalently linked to a lipid or a polyethylene glycol (PEG) as disclosed in U.S. Pat. No. 7,964,571. In some aspects, the PEI backbone is covalently linked to a lipid and a PEG. In some aspects, the lipid and the PEG are directly attached to the PEI backbone by covalent bonds. In some aspects, the lipid is attached to the PEI backbone through a PEG spacer. In some aspects, the PEG has a molecular weight of between 50 to 20,000 Dalton. In some aspects, the molar ratio of PEG to PEI is within a range of 0.1:1 to 500:1. In some aspects, the molar ratio of the lipid to the PEI is within a range of 0.1:1 to 500:1. In some aspects, the lipid is a cholesterol, a cholesterol derivative, a C₁₂ to C₁₈ fatty acid, or a fatty acid derivative. The addition of PEG enhances the stability of the nucleic acid/polymer complexes in the biological milieu and allows for the incorporation of ligands (e.g., a targeting ligand) on to the PPC chain to improve the tissue selectivity of delivery. See U.S. Pat. No. 7,964,571.

In some aspects, the cationic lipopolymer is a PEG:PEI:cholesterol (PPC) lipopolymer, which comprises a PEI backbone covalently linked to cholesterol and PEG. In some aspects, the PEI is covalently linked to cholesterol and PEG, and wherein the average PEG:PEI:cholesterol molar ratio in the cationic lipopolymer is within the range of 1-5 PEG:1 PEI:0.4-1.5 cholesterol. In some aspects, the PEG-PEI-cholesterol (PPC) lipopolymer has an average PEG:PEI:cholesterol ratio of 2.5:1:0.6. In some aspects, the PEI has a linear or branch configuration with a molecular weight of 100 to 500,000 Dalton.

In some aspects, the cationic lipopolymer further comprises a pendant functional moiety selected from the group consisting of: a receptor ligand, a membrane permeating agent, an endosomolytic agent, a nuclear localization sequence, and a pH sensitive endosomolytic peptide.

In some aspects, the cationic lipopolymer further comprises a targeting ligand, wherein the targeting ligand is directly attached to the PEI backbone or is attached through a PEG linker. In some aspects, the targeting ligand is selected from the group consisting of: a sugar moiety, a polypeptide, folate, and an antigen. In some aspects, the sugar moiety is a monosaccharide. In some aspects, the monosaccharide is galactose. In some aspects, the sugar moiety is an oligosaccharide. In some aspects, the polypeptide is a glycoprotein, an antibody, an antibody fragment, a cell receptor, a cytokine receptor, or a growth factor receptor. In some aspects, the growth factor receptor is an epidermal growth factor receptor. In some aspects, the glycoprotein is transferrin or asialoorosomucoid (ASOR). In some aspects, the antigen is a viral antigen, a bacterial antigen, or a parasite antigen.

In some aspects, the cationic lipopolymer is present in an amount sufficient to produce a ratio of amine nitrogen in the cationic polymer to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the cationic lipopolymer to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the cationic lipopolymer to phosphate in the polynucleotide, multicistronic mRNA vector, or DNA plasmid vector is from about 1:10 to about 10:1.

In some aspects, the composition, pharmaceutical composition, or vaccine comprises about 0.1 mg/ml to about 10.0 mg/ml (e.g., about 0.1 mg/ml to about 5 mg/ml; about 0.5 mg/ml to about 10 mg/ml; or about 0.5 mg/ml to about 5 mg/ml) nucleic acid complexed with the cationic polymer. In some aspects, the composition, pharmaceutical composition, or vaccine comprises about 1 mg/ml to about 10 mg/ml (e.g., about 1 mg/ml to about 6 mg/ml; about 2 mg/ml to about 6 mg/ml; about 5 mg/ml to about 10 mg/ml; or about 6 mg/ml to about 10 mg/ml) nucleic acid complexed with the cationic polymer.

### VI.3 Lipopolyamines and Derivatives Thereof

In some aspects, the delivery component of the composition, pharmaceutical composition or vaccine comprises a lipopolyamine with the following formula:

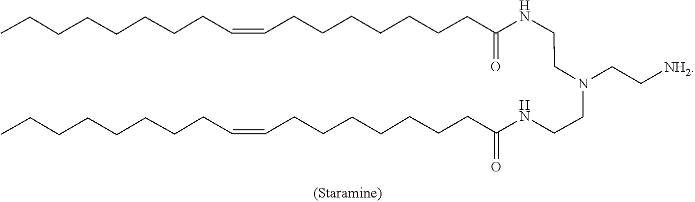 [SMILES: CCCCCCCC/C=C\CCCCCCCC(=O)NCCN(CCN)CCNC(=O)CCCCCCC/C=C\CCCCCCCC]

In some aspects, the delivery component comprises a mixture of the lipopolyamine and an alkylated derivative of the lipopolyamine. In some aspects, the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride. In some aspects, the alkylated derivative of the lipopolyamine has the following formula:

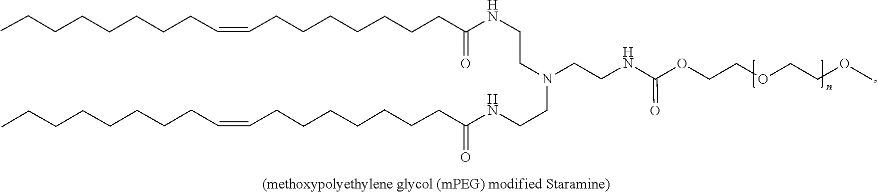

wherein n is an integer from 10 to 100 repeating units containing 2-5 carbon atoms each. In some aspects, the alkylated derivative of the lipopolyamine has the following formula:

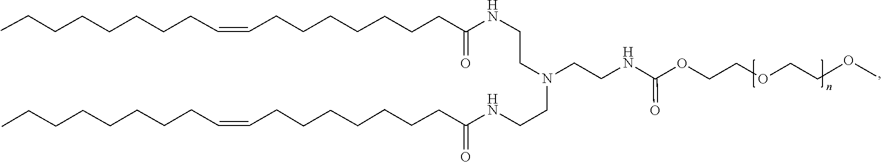

wherein n=11 (Staramine-mPEG515). In some aspects, the alkylated derivative of the lipopolyamine has the following formula:

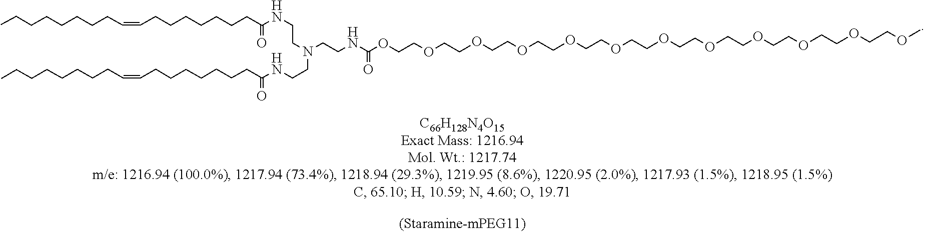 [SMILES: CCCCCCCC/C=C\CCCCCCCC(=O)NCCN(CCNC(=O)CCCCCCC/C=C\CCCCCCCC)CCNC(=O)OCCOCCOCCOCCOCCOCCOCCOCCOCCOCCOCCOC]

In some aspects, the ratio of the lipopolyamine to the alkylated derivative of the lipopolyamine in the mixture is 1:1 to 10:1. In some aspects, the lipopolyamine is present in an amount sufficient to produce a ratio of amine nitrogen in the lipopolyamine to phosphate in the polynucleotide, multicistronic mRNA vector, or DNA plasmid vector from about 0.01:1 to about 50:1 (e.g. about 0.01:1 to about 40:1: about 0.01:1 to about 30:1; about 0.01:1 to about 20:1; about 0.01:1 to about 10:1, or about 0.01:1 to about 5:1). In some aspects, the ratio of amine nitrogen in the lipopolyamine to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the lipopolyamine to phosphate in the polynucleotide, multicistronic mRNA vector, or DNA plasmid vector is from about 1:10 to about 10:1.

In some aspects, the delivery component of the composition, pharmaceutical composition, or vaccine comprises a lipopolyamine with the following formula:

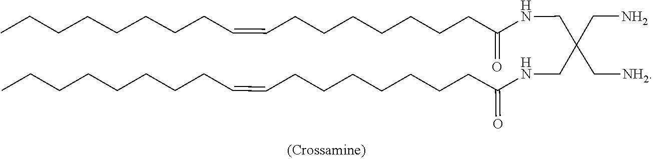 [SMILES: CCCCCCCC/C=C\CCCCCCCC(=O)NCC(CN)(CN)CNC(=O)CCCCCCC/C=C\CCCCCCCC]

In some aspects, the delivery component comprises a mixture of the lipopolyamine and an alkylated derivative of the lipopolyamine. In some aspects, the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride. In some aspects, the ratio of the lipopolyamine to the alkylated derivative of the lipopolyamine in the mixture is 1:1 to 10:1. In some aspects, the lipopolyamine is present in an amount sufficient to produce a ratio of amine nitrogen in the lipopolyamine to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the lipopolyamine to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the lipopolyamine to phosphate in the polynucleotide, multicistronic mRNA vector, or DNA plasmid vector is from about 1:10 to about 10:1.

In some aspects, the composition, pharmaceutical composition, or vaccine comprises about 0.1 mg/ml to about 10.0 mg/ml (e.g., about 0.1 mg/ml to about 5 mg/ml; about 0.5 mg/ml to about 10 mg/ml; or about 0.5 mg/ml to about 5 mg/ml) nucleic acid complexed with the lipopolyamine or derivative thereof. In some aspects, the composition, pharmaceutical composition, or vaccine comprises about 1 mg/ml to about 10 mg/ml (e.g., about 1 mg/ml to about 6 mg/ml; about 2 mg/ml to about 6 mg/ml; about 5 mg/ml to about 10 mg/ml; or about 6 mg/ml to about 10 mg/ml) nucleic acid complexed with the lipopolyamine or derivative thereof.

In some aspects, the polynucleotide, vector, multicistronic mRNA vector, or DNA plasmid vector of the disclosure is complexed with or encapsulated by a delivery component, e.g., a lipopolymer. In some aspects, the polynucleotide, vector, multicistronic mRNA vector, or DNA plasmid vector is encapsulated by a delivery component, e.g., a lipopolymer. In some aspects, at least 20%, at least 30%, at least 40%, at least 50%, at least 60%, at least 70%, at least 75%, at least 80%, at least 85%, at least 90%, at least 95%, or at least 99% of the polynucleotide, vector, multicistronic mRNA vector, or DNA plasmid vector is encapsulated by the delivery component, e.g., a lipopolymer. In some aspects, about 20% to about 100%, about 20% to about 80%, about 20% to about 60%, or about 40% to about 80% of the polynucleotide, vector, multicistronic mRNA vector, or DNA plasmid vector of the disclosure is encapsulated by the delivery component, e.g., a lipopolymer. In some aspects, the delivery component forms a micellular structure.

### VI.4 Poloxamers and Derivatives Thereof

In some aspects, the delivery component comprises a poloxamer back-bone having a metal chelator covalently coupled to at least one terminal end of the poloxamer backbone (e.g. Crown Poloxamer). In some aspects, the metal chelator is coupled to at least two terminal ends of the poloxamer backbone. In some aspects, at least one metal chelator is coupled to a poloxamer. In some aspects, at least two metal chelators are coupled to a poloxamer. In some aspects, 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, or 95-100 metal chelators are coupled to a poloxamer.

In some aspects, the poloxamer backbone is a poloxamer backbone disclosed in U.S. Publ. No. 2010/0004313, which is herein incorporated by reference in its entirety. In some aspects, the metal chelator is a metal chelator disclosed in U.S. Publ. No. 2010/0004313. In some aspects, the delivery component of the composition, pharmaceutical composition, or vaccine comprises a polymer having the following formula:

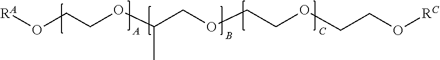

or pharmaceutically acceptable salts thereof, wherein:
A represents an integer from 2 to 141;
B represents an integer from 16 to 67;
C represents an integer from 2 to 141;
R^{A} and R^{C} are the same or different, and are R′-L- or H, wherein at least one of R^{A} and R^{C} is R′-L-;
L is a bond, —CO—, CH₂—O, or —O—CO—; and
R′ is a metal chelator.

In some aspects, the delivery component comprises a poloxamer with the following formula:

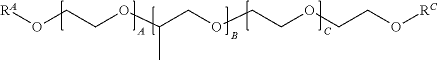

or a pharmaceutically acceptable salt thereof, wherein:
A represents an integer from 2 to 141;
B represents an integer from 16 to 67;
C represents an integer from 2 to 141;
RA and RC are the same or different, and are R′-L- or H;
L is a bond, —CO—, —CH2-O, or —O—CO—; and
R′ is a metal chelator.

In some aspects, at least one of RA and RC is R′-L-.

In some aspects, the R′ is covalently bound to the poloxamer (e.g., where a metal chelator (e.g., a crown) is covalently attached at different density to the poloxamer).

In some aspects, one metal chelator or two or more metal chelators is/are bound to the poloxamer.

In some aspects, 2-100 metal chelators are bound to the poloxamer.

In some aspects, the metal chelator is R^{N}NH—, RN2N—, or (R″—(N(R″)—CH₂CH₂)ₓ)₂—N—CH₂CO—, wherein each x is independently 0-2, and wherein R″ is HO₂C—CH₂—.

In some aspects, the metal chelator is a crown ether selected from the group consisting of 12-crown-4, 15-crown-5, 18-crown-6, 20-crown-6, 21-crown-7, and 24-crown-8. In some aspects, the crown ether is a substituted-crown ether, wherein the substituted-crown ether has:
(1) one or more of the crown ether oxygens independently replaced by NH or S,
(2) one or more of the crown ether —CH₂—CH₂— moieties replaced by C₆H₄—, —C₁₀H₆—, or —C₆H₁₀—,
(3) one or more of the crown ether —CH₂—O—CH₂— moieties replaced by C₄H₂O— or —C₅H₃N—, or
(4) any combination thereof.

In some aspects, the metal chelator is a cryptand, wherein the cryptand is selected from the group consisting of (1,2,2) cryptand, (2,2,2) cryptand, (2,2,3) cryptand, and (2,3,3) cryptand. In some aspects, the cryptand is a substituted-cryptand, wherein the substituted cryptand has:
(1) one or more of the crypthand ether oxygens independently replaced by NH or S,
(2) one or more of the crown ether —CH₂—CH₂— moieties replaced by C₆H₄—, —C₁₀H₆—, or —C₆H₁₀—,
(3) one or more of the crown ether —CH₂—O—CH₂— moieties replaced by C₄H₂O— or —C₅H₃N—, or
(4) any combination thereof.

In some aspects, the delivery component is Crown Poloxamer (aza-crown-linked poloxamer), wherein the Crown Poloxamer comprises a polymer having the following formula:

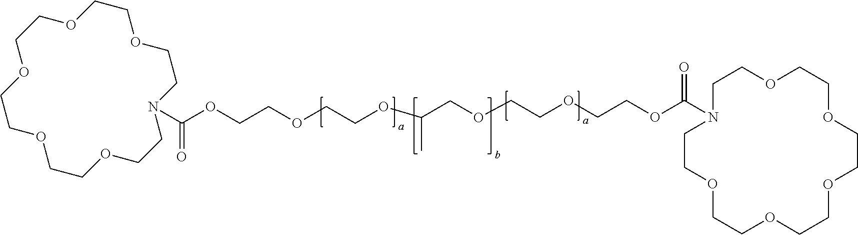

or pharmaceutically acceptable salts thereof, wherein:
a represents an integer of about 10 units; and
b represents an integer of about 21 units; and
   wherein the total molecular weight of the polymer is about 2,000 Da to about 2,200 Da.

In some aspects, the crown poloxamer can be derivatized with a cationic molecule, a ligand, or other chemical entities.

In some aspects, the polymer or poloxamer (e.g., crown poloxamer) is present in a solution with the polynucleotide, multicistronic mRNA vector, or DNA plasmid vector from about 0.1%-about 5% or about 0.5%-about 5%.

In some aspects, the solution is co-formulated with a metal chelator (e.g., where the co-formulated metal chelator is a free metal chelator, which is formulated with the poloxamer (e.g., a non-crown poloxamer)).

In some aspects, the co-formulated metal chelator is present in the solution at a concentration of about 0.1 mg/mL to about 20 mg/mL.

In some aspects, the co-formulated metal chelator is crown ether, a substituted-crown ether, a cryptand, or a substituted-cryptand.

In some aspects, the metal chelator or co-formulated metal chelator is crown ether (Aza-18-crown-6).

In some aspects, the delivery component further comprises benzalkonium chloride (BAK).

In some aspects, the delivery component comprises BD15-12. In some aspects, the ratio of nucleotide to BD15-12 polymer (N:P) is 5:1.

In some aspects, the delivery component comprises Omnifect. In some aspects, the ratio of nucleotide to Omnifect polymer (N:P) is 10:1.

In some aspects, the delivery component comprises Crown Poloxamer (aza-crown-linked poloxamer). In some aspects, the ratio of nucleotide to Crown Poloxamer (N:P) is 5:1. In some aspects, the delivery component comprises Crown Poloxamer and/or a PEG-PEI-cholesterol (PPC) lipopolymer. In some aspects, the delivery component comprises Crown Poloxamer and/or benzalkonium chloride. In some aspects, the delivery component comprises Crown Poloxamer and/or Omnifect. In some aspects, the delivery component comprises Crown Poloxamer and/or a linear polyethyleneimine (LPEI). In some aspects, the delivery component comprises Crown Poloxamer and/or BD15-12.

In some aspects, the delivery component comprises Staramine and/or mPEG modified Staramine. In some aspects, the mPEG modified Staramine is Staramine-mPEG515. In some aspects, the mPEG modified Staramine is Staramine-mPEG11. In some aspects, the ratio of Staramine to mPEG modified Staramine is 10:1. In some aspects, the nucleotide to polymer (N:P) ratio is 5:1. In some aspects, the delivery component comprises Staramine, mPEG modified Staramine, and/or Crown Poloxamer. In some aspects, the delivery component comprises Staramine, Staramine-mPEG515, and/or Crown Poloxamer. In some aspects, the delivery component comprises a Staramine, a Staramine-mPEG11, and/or a Crown Poloxamer.

In some aspects, the compositions of the disclosure are are formulated with about 0.5% crown poloxamer. In some aspects, the composition of the disclosure is formulated with about 0.5 mg/mL AIPO₄. In some aspects, the composition of the disclosure is formulated with crown poloxamer and AIPO₄. In some aspects, the compositions of the disclosure are are formulated with about 0.5% crown poloxamer and about 0.5 mg/mL AIPO₄.

In some aspects, the metal chelator is added directly to a solution comprising (i) the polynucleotide, multicistronic mRNA vector, or DNA plasmid vector as described herein, and (ii) a poloxamer. In some aspects, the metal chelator is present in the solution at concentration of about 0.1 mg/mL to about 20 mg/mL. In some aspects, metal chelator is present in the solution at concentration of about 0.1 mg/mL to about 20 mg/mL, about 0.1 mg/mL to about 1 mg/mL, about 1 mg/mL to about 5 mg/mL, about 5 mg/mL to about 10 mg/mL, about 10 mg/mL to about 15 mg/mL, or about 15 mg/mL to about 20 mg/mL. In some aspects, the metal chelator is crown ether, a substituted-crown ether, a cryptand, or a substituted-cryptand. In some aspects, the metal chelator is crown ether (Aza-18-crown-6).In some aspects, the metal chelator is added directly to a solution comprising (i) the polynucleotide, multicistronic mRNA vector, or DNA plasmid vector as described herein, (ii) a poloxamer, and (iii) an an adjuvant comprising an aluminum or aluminum-salt based adjuvant, a stimulator of interferon genes (STING) agonist, or a combination thereof. In some aspects, the metal chelator is present in the solution at concentration of about 0.1 mg/mL to about 20 mg/mL. In some aspects, metal chelator is present in the solution at concentration of about 0.1 mg/mL to about 20 mg/mL, about 0.1 mg/mL to about 1 mg/mL, about 1 mg/mL to about 5 mg/mL, about 5 mg/mL to about 10 mg/mL, about 10 mg/mL to about 15 mg/mL, or about 15 mg/mL to about 20 mg/mL. In some aspects, the metal chelator is crown ether, a substituted-crown ether, a cryptand, or a substituted-cryptand. In some aspects, the metal chelator is crown ether (Aza-18-crown-6). In some aspects, the adjuvant is AIPO₄.

In some aspects, the metal chelator is added directly to a solution comprising (i) the polynucleotide, multicistronic mRNA vector, or DNA plasmid vector as described herein, (ii) a poloxamer, and (iii) an an adjuvant comprising an aluminum or aluminum-salt based adjuvant, or a combination thereof. In some aspects, the metal chelator is present in the solution at concentration of about 0.1 mg/mL to about 20 mg/mL. In some aspects, metal chelator is present in the solution at concentration of about 0.1 mg/mL to about 20 mg/mL, about 0.1 mg/mL to about 1 mg/mL, about 1 mg/mL to about 5 mg/mL, about 5 mg/mL to about 10 mg/mL, about 10 mg/mL to about 15 mg/mL, or about 15 mg/mL to about 20 mg/mL. In some aspects, the metal chelator is crown ether, a substituted-crown ether, a cryptand, or a substituted-cryptand. In some aspects, the metal chelator is crown ether (Aza-18-crown-6). n some aspects, the adjuvant is AIPO₄.

### VII. Kits and Containers

The present disclosure also features containers comprising any polynucleotide, vector, vaccine, composition, or pharmaceutical composition described and exemplified herein. In some aspects, the container is a glass vial.

The present disclosure also features kits comprising any polynucleotide, vector, vaccine, composition, or pharmaceutical composition described and exemplified herein. The kits can be used to supply polynucleotides, vectors, vaccines, composition, pharmaceutical compositions, and other agents for use in diagnostic, basic research, or therapeutic methods, among others. In some aspects, the kits comprise any one or more of the polynucleotides, vectors, vaccines, composition, or pharmaceutical compositions described or exemplified herein and instructions for using the one or more polynucleotides, vectors, vaccines, composition, or pharmaceutical compositions in a method for inducing an immune response in a subject. In some aspects, the kits comprise any one or more of the polynucleotides, vectors, vaccines, composition, or pharmaceutical compositions described or exemplified herein and instructions for using the one or more polynucleotides, vectors, vaccines, composition, or pharmaceutical compositions in a method for preventing, reducing the incidence of, attenuating or treating a SARS-CoV-2 infection or a monkeypox infection in a subject.

### VIII. Methods of Using the Compositions, Vaccines, and Vectors

The present disclosure also features methods of inducing an immune response in a subject, the method comprising administering an effective amount of any polynucleotide, vector, multicistronic mRNA vector, DNA plasmid vector, composition, or pharmaceutical composition described or exemplified herein to the subject. In some aspects, the immune response is to one or more pathogen antigens disclosed herein, e.g., SARS-CoV-2 viral antigens or monkeypox viral antigens.

In some aspects, the immune response is to one or more antigens comprising one or more viral antigens, one or more bacterial antigens, or one or more parasite antigens.

In some aspects, the immune response is to a bacterial antigen is selected from a Yersinia pestis antigen or a Mycobacterium tuberculosis antigen. In some aspects, the Yersinia pestis antigen is a Yersinia pestis capsular antigen. In some aspects, the Yersinia pestis capsular antigen is F1-Ag or virulence antigen (V-Ag). In some aspects, the Mycobacterium tuberculosis antigen is an Apa antigen, an HP65 antigen, or a rAg85A antigen.

In some aspects, the immune response is to a viral antigen selected from an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a orthopoxvirus antigen, a monkeypox antigen, a vaccinia antigen, a smallpox antigen, a Epstein bar virus antigen, a nipha virus antigen, or a varicella-zoster virus antigen. In some aspects, the enterovirus antigen is an enterovirus 71 (E71) antigen or a coxsackievirus (Cox) protein antigen. In some aspects, the E71 antigen is a E71-VP1 antigen or a glutathione S-transferase (GST)-tagged E71-VP1 antigen. In some aspects, the Cox protein antigen is GST-tagged Cox protein antigen. In some aspects, the HSV antigen is an HSV-1 envelope antigen, an HSV-2 envelope antigen, or an HSV-2 surface glycoprotein antigen. In some aspects, the HSV-2 surface glycoprotein antigen is a gB2 antigen, a gC2 antigen, a gD2 antigen, or a gE2 antigen. In some aspects, the HIV antigen is an Env antigen, a Gag antigen, a Nef antigen, or a Pol antigen. In some aspects, the HPV antigen is a minor capsid protein L2 antigen. In some aspects, the minor capsid protein L2 antigen comprises one or more epitope domains (amino acids 10-36 and/or amino acids 65-89) of minor capsid protein L2. In some aspects, the HPV antigen is a human papillomavirus type 16 Regulatory protein E2 antigen, a human papillomavirus type 16 Protein E6 antigen, a human papillomavirus type 16 Protein E7 antigen, a human papillomavirus type 18 Regulatory protein E2 antigen, a human papillomavirus type 18 Protein E6 antigen, a human papillomavirus type 18 Protein E7 antigen, a human papillomavirus type 6a Regulatory protein E2 antigen, a human papillomavirus type 6a Protein E6 antigen, a human papillomavirus type 6a Protein E7 antigen, a human papillomavirus 11 Regulatory protein E2 antigen, a human papillomavirus 11 Protein E6 antigen, a human papillomavirus 11 Protein E7 antigen, antigenic fragments thereof, or any combinations thereof. In some aspects, the HCV antigen is a nonstructural 3 (NS3) antigen, a hepatitis C virus genotype 1a Genome polyprotein antigen, a hepatitis C virus genotype 1b Genome polyprotein antigen, a hepatitis C virus genotype 2a Genome polyprotein antigen, a hepatitis C virus genotype 3a Genome polyprotein antigen, antigenic fragments thereof, or any combinations thereof. In some aspects, the RSV antigen is an F antigen or a G antigen. In some aspects, the Dengue virus antigen is an E protein antigen, an E protein domain III (EDIII) antigen, a non-structural protein 1 (NS1) antigen, or a DEN-80E antigen. In some aspects, the Ebola virus antigen is a spike glycoprotein (GB) antigen, a VP24 antigen, a VP40 antigen, a nucleoprotein (NP) antigen, a VP30 antigen, or a VP35 antigen. In some aspects the Zika virus antigen is an envelope domain III antigen or a CKD antigen. In some aspects, the Chikungunya virus antigen is an E1 glycoprotein subunit antigen, the MHC class I epitope PPFGAGRPGQFGDI (SEQ ID NO: 34), the MHC class I epitope TAECKDKNL (SEQ ID NO: 35), or the MHC class II epitope VRYKCNCGG (SEQ ID NO: 36). In some aspects, the measles virus antigen is a hemagglutinin protein MV-H antigen or a fusion protein MV-F antigen. In some aspects, the MERS-CoV antigen is a spike (S) protein antigen, an antigen from the receptor-binding domain of the S protein, or an antigen from the membrane fusion domain of the S protein. In some aspects, the SARS-CoV antigen is a spike (S) protein antigen, an antigen from the receptor binding domain of the S protein, an antigen from the membrane fusion domain of the S protein, an envelope (E) protein antigen, or an M protein antigen. In some aspects, the monkeypox antigen is a A35R protein antigen, a H3L protein antigen, or a L1R protein antigen. In some aspects, the Epstein-Barr virus antigen is an Epstein-Barr virus (strain B95-8) nuclear antigen 1 antigen, an Epstein-Barr virus (strain B95-8) Envelope glycoprotein B antigen, an Epstein-Barr virus (strain B95-8) Envelope glycoprotein H antigen, an Epstein-Barr virus (strain B95-8) Envelope glycoprotein GP350 antigen, an Epstein-Barr virus (strain B95-8) Latent membrane protein 1 antigen, or an Epstein-Barr virus (strain B95-8) Latent membrane protein 2 antigen. In some aspects, the Vaccinia virus is a Vaccinia virus (strain Western Reserve) Protein A27 antigen, a Vaccinia virus (strain Western Reserve) EEV membrane phosphoglycoprotein antigen, a Vaccinia virus B5R (Fragment) antigen, a Vaccinia virus Envelope protein H3 antigen, a Vaccinia virus (strain Western Reserve) IMV membrane protein antigen. In some aspects, the Nipah virus is a Nipah virus Fusion glycoprotein FO antigen, a Nipah virus Glycoprotein G antigen. In some aspects, the Varicella-zoster virus is a Varicella-zoster virus (strain Dumas) Envelope glycoprotein E antigen.

In some aspects, the immune response is to one or more influenza virus antigens from any influenza virus type or subtype. In some aspects, the one or more influenza virus antigens are selected from the group consisting of: an influenza virus hemagglutinin (HA) antigen, an influenza virus neuraminidase (NA) antigen, an influenza virus matrix-2 (M2) protein antigen, antigenic fragments thereof, and any combination thereof. In some aspects, the one or more influenza virus antigens are derived from influenza virus type A, type B, type C, type D, or any combination thereof. In some aspects, the one or more influenza virus antigens are derived from influenza virus type A. In some aspects, the one or more influenza virus antigens derived from influenza virus type A have (a) a HA subtype selected from H1 through H18 or any combination thereof and (b) a NA subtype selected from N1 through N11 or any combination thereof. In some aspects, the one or more influenza virus antigens derived from influenza virus type A, subtype H1N1; influenza virus type A, subtype H2N2; influenza virus type A, subtype H3N2; influenza virus type A, subtype H5N1; influenza virus type A, subtype H7N7; influenza virus type A, subtype H7N9; influenza virus type A, subtype H9N2; or any combination thereof. In some aspects, the one or more influenza virus antigens are derived from influenza virus type A, subtype H1N1; influenza virus type A, subtype H3N2; or the combination thereof. In some aspects, the one or more influenza virus antigens are derived from influenza virus type B. In some aspects, the immune response is to one or more SARS-CoV-2 antigens or antigenic fragments thereof disclosed herein and one or more influenza virus antigens or antigenic fragments thereof disclosed herein. In some aspects, the immune response is to one or more monkeypox antigens or antigenic fragments thereof disclosed herein and one or more influenza virus antigens or antigenic fragments thereof disclosed herein. In some aspects, the immune response is to a parasite antigen, wherein the parasite antigen is a protozoan antigen. In some aspects, the immune response is to a parasite antigen selected from the group consisting of a Toxoplasma gondii antigen or a Plasmodium falciparum antigen. In some aspects, the Toxoplasma gondii antigen is antigen MIC8. In some aspects, the Plasmodium falciparum antigen is a SERA5 polypeptide antigen, or a circumsporozite protein antigen. In some aspects, the immune response is to a parasite antigen, wherein the parasite antigen is a parasitic or pathogenic fungus antigen. In some aspects, the parasitic or pathogenic fungus antigen is selected from the group consisting of a Candida spp. antigen (e.g., a Candida albicans antigen, a Candida glabrata antigen, a Candida parapsilosis antigen, a Candida tropicalis antigen, a Candida lusitaniae antigen, a Candida krusei antigen), a Pneumocystis spp. antigen, a Malassezia spp. antigen (e.g., a Malassezia furfur antigen), an Aspergillus fumigatus antigen, a Cryptococcus spp. antigen (e.g., a Cryptococcus neoformans antigen, a Cryptococcus gattii antigen), a Histoplasma capsulatum antigen, a Blastomyces dermatitidis antigen, a Paracoccidioides spp. antigen (e.g., a Paracoccidioides brasiliensis antigen, a Paracoccidioides lutzii antigen), a Coccidioides spp. antigen (e.g., a Coccidioides immitis antigen, a Coccidioides posadasii antigen), a Penicillium marneffei antigen, a Sporothrix schenckii antigen, a Trichosporon asahii antigen, a Fusarium spp. antigen (e.g., a Fusarium solanum antigen, a Fusarium oxysporum antigen), a Nectria spp. antigen, a Pseudoallescheria boydii antigen, a Cladophialphora bantianum antigen, a Ramichloridium spp. antigen, a Dactylaria gallopava antigen, an Exophiala spp. antigen (e.g., an Exophiala jeanselmei antigen, an Exophiala dermatitidis antigen), a Curvularia spp. antigen, a Bipolaris spp. antigen, an Alternaria spp. antigen, a Lacazia loboi antigen, a Conidiobolus spp. antigen (e.g., a Conidiobolus coronatus antigen, a Conidiobolus incongruus antigen), and any combination thereof.

Also provided herein is a method of preventing, reducing the incidence of, attenuating or treating a virus (e.g., SARS-CoV-2 or monkeypox), a bacteria or a parasite infection in a subject.

The present disclosure also features methods of preventing, reducing the incidence of, attenuating or treating a virus (e.g., SARS-CoV-2 or monkeypox), a bacteria or a parasite infection in a subject, the method comprising administering an effective amount of any polynucleotide, vector, multicistronic mRNA vector, DNA plasmid vector, composition, or pharmaceutical composition described or exemplified herein to the subject.

In some aspects, the methods of the disclosure are directed to administration of a composition, pharmaceutical composition, or vaccine disclosed herein.

The process of administration can be varied, depending on the agent, or agents, and the desired effect. Thus, the process of administration involves administering a therapeutic agent (e.g., any composition or pharmaceutical composition, polynucleotide, vector, vaccine, disclosed herein) to a patient in need of such treatment. Methods of delivering compositions comprising DNA vaccines are described in U.S. Pat. Nos. 4,945,050 and 5,036,006.

Administration can be accomplished by any means appropriate for the therapeutic agent, for example, by parenteral, mucosal, pulmonary, topical, catheter-based, or oral means of delivery. Parenteral delivery can include for example, subcutaneous, intravenous, intramuscular, transdermal, intra-arterial, intraperitoneal, intralymphatic, and injection into the tissue of an organ. Mucosal delivery can include, for example, intranasal delivery, preferably administered into the airways of a patient, i.e., nose, sinus, throat, lung, for example, as nose drops, by nebulization, vaporization, or other methods known in the art. Oral or intranasal delivery can include the administration of a propellant. Pulmonary delivery can include inhalation of the agent. Catheter-based delivery can include delivery by iontropheretic catheter-based delivery. Oral delivery can include delivery of a coated pill, or administration of a liquid by mouth. Administration can generally also include delivery with a pharmaceutically acceptable carrier, such as, for example, a buffer, a polypeptide, a peptide, a polysaccharide conjugate, a liposome, and/or a lipid, according to methods known in the art.

Pulmonary delivery can include inhalation of the agent. Catheter-based delivery can include delivery by iontropheretic catheter-based delivery. Oral delivery can include delivery of a coated pill, or administration of a liquid by mouth. Administration can generally also include delivery with a pharmaceutically acceptable carrier, such as, for example, a buffer, a polypeptide, a peptide, a polysaccharide conjugate, a liposome, and/or a lipid, according to methods known in the art.

In some aspects, the viral vectors of the present disclosure are administered in an amount approximately corresponding to 10² to 10¹⁴ PFU, 10⁵ to 10¹² PFU, or 10⁶ to 10¹⁰ PFU per subject, calculated as the PFU of the viral vector. In some aspects, the viral vectors of the present disclosure are administered by directly injecting a viral vector suspension prepared by suspending the viral vector in PBS (phosphate buffered saline) or saline into a local site (e.g., into the lung tissue, liver, muscle or brain), by nasal or respiratory inhalation, or by intravascular (e.g., intra-arterial, intravenous, and portal venous), intralymphatic, subcutaneous, intracutaneous, intradermal, transdermal, or intraperitoneal administration.

Certain aspects of the disclosure relate to method of administering to a subject a composition comprising (i) a vector (e.g., a multicistronic DNA plasmid vector or a multicistronic messenger RNA (mRNA) vector) comprising a nucleic acid sequence encoding one or more viral antigens (e.g., a SARS CoV-2 antigen or monkeypox antigen); (ii) a delivery component (e.g., a cationic polymer, a poly-inosinic-polycytidylic acid, or a poloxamer); and (iii) an adjuvant comprising an aluminum or aluminum-salt based adjuvant, a stimulator of interferon genes (STING) agonist, or a combination thereof. In some aspects, the adjuvant comprises an aluminum or aluminum-salt based adjuvant and a STING agonist. In some aspects, the aluminum or aluminum-salt based adjuvant comprises an aluminum oxyhydroxide, an aluminum hydroxide, an aluminum phosphate, or any combination thereof. In some aspects, the STING agonist is cyclic guanosine monophosphate (cGMP), cyclic adenosine monophosphate (cAMP), or cyclic guanosine monophosphate-adenosine monophosphate (cGAMP).

In some aspects, the vector further comprises a nucleic acid sequence encoding one or more immune modifier proteins. In some aspects, the vector comprises a nucleic acid sequence encoding a SARS CoV-2 antigen and, optionally, a second viral antigen. In some aspects, the vector comprises a nucleic acid sequence encoding a monkeypox antigen and, optionally, a second viral antigen. Some aspects relate to methods of eliciting humoral and/or cellular immune response against a pathogen (e.g., SARS-CoV-2 or monkeypox) challenge or infection following in vivo administration of a vector or composition of the disclosure.

Some aspects relate to methods of eliciting humoral and/or cellular immune response against a pathogen (e.g., SARS-CoV-2 or monkeypox) challenge or infection following in vivo administration of a multicistronic DNA plasmid, a multicistronic mRNA vector, or a composition comprising the same of the disclosure.

Certain aspects of the disclosure relate to administering compositions including (i) a DNA plasmid vector comprising a DNA sequence of one or more antigens; (ii) a delivery component, such as a synthetic non-viral DNA carrier (e.g., a cationic polymer, a poly-inosinic-polycytidylic acid, or a poloxamer); and (iii) an adjuvant comprising an aluminum or aluminum-salt based adjuvant, a stimulator of interferon genes (STING) agonist, or a combination thereof.

The delivery component of the compositions disclosed herein may comprise any combination of delivery components disclosed herein.

In some aspects, the pathogen is a virus, a bacterium or a parasite. In some aspects, the one or more antigens comprise one or more viral antigens, one or more bacterial antigens, or one or more parasite antigens. In some aspects the one or more viral antigens, one or more bacterial antigens, or one or more parasite antigens comprise two or more variants of the same antigen (e.g., two or more variants of an antigen from different strains of the virus, bacterium, or parasite).

In some aspects, the viral antigen is selected from the group consisting of: an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a orthopoxvirus antigen, a monkeypox antigen, a vaccinia antigen, a smallpox antigen, a Epstein bar virus antigen, a nipha virus antigen, or a varicella-zoster virus antigen, any antigenic fragments thereof, or any combination thereof. In some aspects, the enterovirus antigen is an enterovirus 71 (E71) antigen, a coxsackievirus (Cox) protein antigen, any antigenic fragments thereof, or any combination thereof. In some aspects, the E71 antigen is a E71-VP1 antigen, a glutathione S-transferase (GST)-tagged E71-VP1 antigen, any antigenic fragments thereof, or any combination thereof. In some aspects, the Cox protein antigen is GST-tagged Cox protein antigen. In some aspects, the HSV antigen is an HSV-1 envelope antigen, an HSV-2 envelope antigen, an HSV-2 surface glycoprotein antigen, any antigenic fragments thereof, or any combination thereof. In some aspects, the HSV-2 surface glycoprotein antigen is a gB2 antigen, a gC2 antigen, a gD2 antigen, a gE2 antigen, any antigenic fragments thereof, or any combination thereof. In some aspects, the HIV antigen is an Env antigen, a Gag antigen, a Nef antigen, a Pol antigen, any antigenic fragments thereof, or any combination thereof. In some aspects, the HPV antigen is a minor capsid protein L2 antigen. In some aspects, the minor capsid protein L2 antigen comprises one or more epitope domains (amino acids 10-36 and/or amino acids 65-89) of minor capsid protein L2. In some aspects, the HPV antigen is a human papillomavirus type 16 Regulatory protein E2 antigen, a human papillomavirus type 16 Protein E6 antigen, a human papillomavirus type 16 Protein E7 antigen, a human papillomavirus type 18 Regulatory protein E2 antigen, a human papillomavirus type 18 Protein E6 antigen, a human papillomavirus type 18 Protein E7 antigen, a human papillomavirus type 6a Regulatory protein E2 antigen, a human papillomavirus type 6a Protein E6 antigen, a human papillomavirus type 6a Protein E7 antigen, a human papillomavirus 11 Regulatory protein E2 antigen, a human papillomavirus 11 Protein E6 antigen, a human papillomavirus 11 Protein E7 antigen, antigenic fragments thereof, or any combinations thereof. In some aspects, the HCV antigen is a nonstructural 3 (NS3) antigen, a hepatitis C virus genotype 1a Genome polyprotein antigen, a hepatitis C virus genotype 1b Genome polyprotein antigen, a hepatitis C virus genotype 2a Genome polyprotein antigen, a hepatitis C virus genotype 3a Genome polyprotein antigen, antigenic fragments thereof, or any combinations thereof. In some aspects, the RSV antigen is an F antigen, a G antigen, any antigenic fragments thereof, or a combination thereof. In some aspects, the Dengue virus antigen is an E protein antigen, an E protein domain III (EDIII) antigen, a non-structural protein 1 (NS1) antigen, a DEN-80E antigen, any antigenic fragments thereof, or any combination thereof. In some aspects, the Ebola virus antigen is a spike glycoprotein (GB) antigen, a VP24 antigen, a VP40 antigen, a nucleoprotein (NP) antigen, a VP30 antigen, a VP35 antigen, any antigenic fragments thereof, or any combination thereof. In some aspects the Zika virus antigen is an envelope domain III antigen, a CKD antigen, any antigenic fragments thereof, or any combination thereof. In some aspects, the Chikungunya virus antigen is an E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), any antigenic fragments thereof, or any combination thereof. In some aspects, the measles virus antigen is a hemagglutinin protein MV-H antigen, a fusion protein MV-F antigen, any antigenic fragments thereof, or any combination thereof. In some aspects, the MERS-CoV antigen is a spike (S) protein antigen, an antigen from the receptor-binding domain of the S protein, an antigen from the membrane fusion domain of the S protein, any antigenic fragments thereof, or any combination thereof. In some aspects, the SARS-CoV antigen is a spike (S) protein antigen, an antigen from the receptor binding domain of the S protein, an antigen from the membrane fusion domain of the S protein, an envelope (E) protein antigen, an M protein antigen, any antigenic fragments thereof, or any combinations thereof. In some aspects, the monkeypox antigen is a A35R protein antigen, a H3L protein antigen, or a L1R protein antigen. In some aspects, the Epstein-Barr virus antigen is an Epstein-Barr virus (strain B95-8) nuclear antigen 1 antigen, an Epstein-Barr virus (strain B95-8) Envelope glycoprotein B antigen, an Epstein-Barr virus (strain B95-8) Envelope glycoprotein H antigen, an Epstein-Barr virus (strain B95-8) Envelope glycoprotein GP350 antigen, an Epstein-Barr virus (strain B95-8) Latent membrane protein 1 antigen, or an Epstein-Barr virus (strain B95-8) Latent membrane protein 2 antigen. In some aspects, the Vaccinia virus is a Vaccinia virus (strain Western Reserve) Protein A27 antigen, a Vaccinia virus (strain Western Reserve) EEV membrane phosphoglycoprotein antigen, a Vaccinia virus B5R (Fragment) antigen, a Vaccinia virus Envelope protein H3 antigen, a Vaccinia virus (strain Western Reserve) IMV membrane protein antigen. In some aspects, the Nipah virus is a Nipah virus Fusion glycoprotein FO antigen, a Nipah virus Glycoprotein G antigen. In some aspects, the Varicella-zoster virus is a Varicella-zoster virus (strain Dumas) Envelope glycoprotein E antigen.

In some aspects, the one or more viral antigens comprise one or more influenza virus antigens from any influenza virus type or subtype. In some aspects, the one or more influenza virus antigens are selected from the group consisting of: an influenza virus hemagglutinin (HA) antigen, an influenza virus neuraminidase (NA) antigen, an influenza virus matrix-2 (M2) protein antigen, antigenic fragments thereof, and any combination thereof. In some aspects, the one or more influenza virus antigens are derived from influenza virus type A, type B, type C, type D, or any combination thereof. In some aspects, the one or more influenza virus antigens are derived from influenza virus type A. In some aspects, the one or more influenza virus antigens derived from influenza virus type A have (a) a HA subtype selected from H1 through H18 or any combination thereof and (b) a NA subtype selected from N1 through N11 or any combination thereof. In some aspects, the one or more influenza virus antigens derived from influenza virus type A, subtype H1N1; influenza virus type A, subtype H2N2; influenza virus type A, subtype H3N2; influenza virus type A, subtype H5N1; influenza virus type A, subtype H7N7; influenza virus type A, subtype H7N9; influenza virus type A, subtype H9N2; or any combination thereof. In some aspects, the one or more influenza virus antigens are derived from influenza virus type A, subtype H1N1; influenza virus type A, subtype H3N2; or the combination thereof. In some aspects, the one or more influenza virus antigens are derived from influenza virus type B. In some aspects, the one or more viral antigens comprise one or more SARS-CoV-2 antigens or antigenic fragments thereof disclosed herein and one or more influenza virus antigens or antigenic fragments thereof disclosed herein. In some aspects, the one or more viral antigens comprise one or more monkeypox antigens or antigenic fragments thereof disclosed herein and one or more influenza virus antigens or antigenic fragments thereof disclosed herein. In some aspects, the parasite antigen is a protozoan antigen. In some aspects, the parasite antigen is selected from the group consisting of a Toxoplasma gondii antigen, a Plasmodium falciparum antigen, any antigenic fragments thereof, or any combination thereof. In some aspects, the Toxoplasma gondii antigen is antigen MIC8. In some aspects, the Plasmodium falciparum antigen is a SERA5 polypeptide antigen, a circumsporozite protein antigen, any antigenic fragments thereof, or any combinations thereof. In some aspects, the parasite antigen is a parasitic or pathogenic fungus antigen. In some aspects, the parasitic or pathogenic fungus antigen is selected from the group consisting of a Candida spp. antigen (e.g., a Candida albicans antigen, a Candida glabrata antigen, a Candida parapsilosis antigen, a Candida tropicalis antigen, a Candida lusitaniae antigen, a Candida krusei antigen), a Pneumocystis spp. antigen, a Malassezia spp. antigen (e.g., a Malassezia furfur antigen), an Aspergillus fumigatus antigen, a Cryptococcus spp. antigen (e.g., a Cryptococcus neoformans antigen, a Cryptococcus gattii antigen), a Histoplasma capsulatum antigen, a Blastomyces dermatitidis antigen, a Paracoccidioides spp. antigen (e.g., a Paracoccidioides brasiliensis antigen, a Paracoccidioides lutzii antigen), a Coccidioides spp. antigen (e.g., a Coccidioides immitis antigen, a Coccidioides posadasii antigen), a Penicillium marneffei antigen, a Sporothrix schenckii antigen, a Trichosporon asahii antigen, a Fusarium spp. antigen (e.g., a Fusarium solanum antigen, a Fusarium oxysporum antigen), a Nectria spp. antigen, a Pseudoallescheria boydii antigen, a Cladophialphora bantianum antigen, a Ramichloridium spp. antigen, a Dactylaria gallopava antigen, an Exophiala spp. antigen (e.g., an Exophiala jeanselmei antigen, an Exophiala dermatitidis antigen), a Curvularia spp. antigen, a Bipolaris spp. antigen, an Alternaria spp. antigen, a Lacazia loboi antigen, a Conidiobolus spp. antigen (e.g., a Conidiobolus coronatus antigen, a Conidiobolus incongruus antigen), and any combination thereof.

In some aspects, the one or more antigens are viral antigens. In some aspects, the one or more viral antigens comprises a SARS-CoV-2 antigen or an antigenic fragment thereof. In some aspects, the one or more viral antigens comprise one or more viral antigens (e.g., a S protein, a S1 subunit of a S protein, a RBD of a S protein, a membrane fusion domain of a S protein, a M protein, an E protein, or an antigenic fragment thereof) from one or more SARS-CoV-2 strains selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); a Zeta strain (e.g., strain P.2); and any combination thereof. In some aspects, the one or more viral antigens are one or more SARS-CoV-2 S proteins or antigenic fragments thereof from one or more SARS-CoV-2 strains selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); a Zeta strain (e.g., strain P.2); and any combination thereof. In some aspects, the one or more viral antigens comprise one or more viral antigens (e.g., a S protein, a S1 subunit of a S protein, a RBD of a S protein, a M protein, or an antigenic fragment thereof) from one or more SARS-CoV-2 strains selected from the group consisting of: B.1.1.7, Q.1, Q.2, Q.3, Q.4, Q.5, Q.6, Q.7, Q.8, B.1.351, B.1.351.2, B.1.351.3, B.1.617.2, AY.1 sublineages, P.1, P.1.1, P.1.2, B.1.427, B.1.429, B.1.525, B.1.526, B.1.617.1, B.1.617.3, B.1.621, B.1.621.1, P.2, and any combination thereof. In some aspects, the one or more viral antigens are one or more SARS-CoV-2 S proteins or antigenic fragments thereof from one or more SARS-CoV-2 strains selected from the group consisting of: B.1.1.7, Q.1, Q.2, Q.3, Q.4, Q.5, Q.6, Q.7, Q.8, B.1.351, B.1.351.2, B.1.351.3, B.1.617.2, AY.1 sublineages, P.1, P.1.1, P.1.2, B.1.427, B.1.429, B.1.525, B.1.526, B.1.617.1, B.1.617.3, B.1.621, B.1.621.1, P.2, and any combination thereof. In some aspects, the one or more viral antigens comprises a monkeypox antigen or an antigenic fragment thereof.

In some aspects, the one or more viral antigens comprise a SARS-CoV-2 S protein or antigenic fragment thereof from an Alpha SARS-CoV-2 strain, wherein the SARS-CoV-2 S protein or antigenic fragment thereof comprises the amino acid sequence of SEQ ID NO: 2 or SEQ ID NO: 4 with one or mutations selected from AH69-V70, A144, E484K, N501Y, A570D, D614G, P681H, T716I, S982A, and D1118H wherein the amino acid locations correspond to SEQ ID NO: 2 or SEQ ID NO: 4. In some aspects, the one or more viral antigens comprise a SARS-CoV-2 S protein or antigenic fragment thereof from a Beta SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, D80A, D215G, AL241-S243, K417N, E484K, N501Y, D614G, and A701V, wherein the amino acid locations correspond to SEQ ID NO: 2 or SEQ ID NO: 4. In some aspects, the one or more viral antigens comprise a SARS-CoV-2 S protein or antigenic fragment thereof from a Gamma SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, T20N, P26S, D138Y, R190S, K417T, E484K, N501Y, D614G, H655Y, T1027I, and V1176F, wherein the amino acid locations correspond to SEQ ID NO: 2 or SEQ ID NO: 4. In some aspects, the one or more viral antigens comprise a SARS-CoV-2 S protein or antigenic fragment thereof from a Delta SARS-CoV-2 strain, wherein the SARS-CoV-2 S protein or antigenic fragment thereof comprises the amino acid sequence of SEQ ID NO: 2 or SEQ ID NO: 4 with one or mutations selected from T19R, AD 119-F120, AE156-F157, R158G, L452R, T478K, D614G, P681R, and D950N, wherein the amino acid locations correspond to SEQ ID NO: 2 or SEQ ID NO: 4. In some aspects, the one or more viral antigens comprise a SARS-CoV-2 S protein or antigenic fragment thereof from a Kappa SARS-CoV-2 strain, wherein the SARS-CoV-2 S protein or antigenic fragment thereof comprises the amino acid sequence of SEQ ID NO: 2 or SEQ ID NO: 4 with one or mutations selected from E154K, L452R, E484Q, D614G, P681R, and Q1071H, wherein the amino acid locations correspond to SEQ ID NO: 2 or SEQ ID NO: 4. In some aspects, the one or more viral antigens comprise a SARS-CoV-2 S protein or antigenic fragment thereof from an Eta SARS-CoV-2 strain, wherein the SARS-CoV-2 S protein or antigenic fragment thereof comprises the amino acid sequence of SEQ ID NO: 2 or SEQ ID NO: 4 with one or mutations selected from Q52R, A67V, AH69-V70, AY144, E484K, D614G, Q677H, and F888L, wherein the amino acid locations correspond to SEQ ID NO: 2 or SEQ ID NO: 4. In some aspects, the one or more viral antigens comprise a SARS-CoV-2 S protein or antigenic fragment thereof from an Iota SARS-CoV-2 strain, wherein the SARS-CoV-2 S protein or antigenic fragment thereof comprises the amino acid sequence of SEQ ID NO: 2 or SEQ ID NO: 4 with one or mutations selected from L5F, T95I, D253G, E484K, D614G, and A701V, wherein the amino acid locations correspond to SEQ ID NO: 2 or SEQ ID NO: 4. In some aspects, the one or more viral antigens comprise a SARS-CoV-2 S protein or antigenic fragment thereof from a Lambda SARS-CoV-2 strain, wherein the SARS-CoV-2 S protein or antigenic fragment thereof comprises the amino acid sequence of SEQ ID NO: 2 or SEQ ID NO: 4 with one or mutations selected from G75V, T76I, AR246-G252, D253N, L452Q, F490S, D614G, and T859N, wherein the amino acid locations correspond to SEQ ID NO: 2 or SEQ ID NO: 4. In some aspects, the one or more viral antigens comprise a SARS-CoV-2 S protein or antigenic fragment thereof from a Mu SARS-CoV-2 strain, wherein the SARS-CoV-2 S protein or antigenic fragment thereof comprises the amino acid sequence of SEQ ID NO: 2 or SEQ ID NO: 4 with one or mutations selected from T95I, Y144S, Y145N, R346K, E484K, N501Y, D614G, P681H, and D950N, wherein the amino acid locations correspond to SEQ ID NO: 2 or SEQ ID NO: 4. In some aspects, the one or more viral antigens comprise a SARS-CoV-2 S protein or antigenic fragment thereof from an Epsilon SARS-CoV-2 strain, wherein the SARS-CoV-2 S protein or antigenic fragment thereof comprises the amino acid sequence of SEQ ID NO: 2 or SEQ ID NO: 4 with one or mutations selected from S131, W152C, L452R, and D614G, wherein the amino acid locations correspond to SEQ ID NO: 2 or SEQ ID NO: 4.

In some aspects, the one or more viral antigens comprise at least two viral antigens (e.g., two or more antigens or antigenic fragments from a S protein, a S1 subunit of a S protein, a RBD of a S protein, a membrane fusion domain of a S protein, a M protein, an E protein, or any combination thereof) from two or more different strains of SARS-CoV-2. In some aspects, the one or more viral antigens comprise at least two SARS-CoV-2 S proteins or antigenic fragments thereof from two or more different strains of SARS-CoV-2. In some aspects, the two or more different strains of SARS-CoV-2 are selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); a Zeta strain (e.g., strain P.2); and any combination thereof. In some aspects, the two or more different strains of SARS-CoV-2 are selected from the group consisting of: B.1.1.7, Q.1, Q.2, Q.3, Q.4, Q.5, Q.6, Q.7, Q.8, B.1.351, B.1.351.2, B.1.351.3, B.1.617.2, AY.1 sublineages, P.1, P.1.1, P.1.2, B.1.427, B.1.429, B.1.525, B.1.526, B.1.617.1, B.1.617.3, B.1.621, B.1.621.1, P.2, and any combination thereof. In some aspects, the at least two SARS-CoV-2 S proteins or antigenic fragments thereof from two or more different strains of SARS-CoV-2 comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety.

Mutations and viral sequence data for the SARS-CoV-2 variants are publically available at the CoVariants website (https://covariants.org/) and the National Center for Biotechnology Information (NCBI) website (https://www.ncbi.nlm.nih.gov/labs/virus/vssi/#/sars-cov-2), each of which is incorporated by reference in its entirety. In some aspects, the SARS-CoV-2 S proteins or antigenic fragments thereof disclosed herein include a SARS-CoV-2 S protein or antigenic fragment thereof from an Alpha SARS-CoV-2 strain, wherein the SARS-CoV-2 S protein or antigenic fragment thereof comprises the amino acid sequence of SEQ ID NO: 2 or SEQ ID NO: 4 with one or mutations selected from AH69-V70, A144, E484K, N501Y, A570D, D614G, P681H, T716I, S982A, and D1118H wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from a Beta SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, D80A, D215G, AL241-S243, K417N, E484K, N501Y, D614G, and A701V, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from a Gamma SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, T20N, P26S, D138Y, R190S, K417T, E484K, N501Y, D614G, H655Y, T1027I, and V1176F, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from a Delta SARS-CoV-2 strain, wherein the SARS-CoV-2 S protein or antigenic fragment thereof comprises the amino acid sequence of SEQ ID NO: 2 or SEQ ID NO: 4 with one or mutations selected from T19R, AD119-F120, AE156-F157, R158G, L452R, T478K, D614G, P681R, and D950N, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from a Kappa SARS-CoV-2 strain, wherein the SARS-CoV-2 S protein or antigenic fragment thereof comprises the amino acid sequence of SEQ ID NO: 2 or SEQ ID NO: 4 with one or mutations selected from E154K, L452R, E484Q, D614G, P681R, and Q1071H, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from an Eta SARS-CoV-2 strain, wherein the SARS-CoV-2 S protein or antigenic fragment thereof comprises the amino acid sequence of SEQ ID NO: 2 or SEQ ID NO: 4 with one or mutations selected from Q52R, A67V, AH69-V70, AY144, E484K, D614G, Q677H, and F888L, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from an Iota SARS-CoV-2 strain, wherein the SARS-CoV-2 S protein or antigenic fragment thereof comprises the amino acid sequence of SEQ ID NO: 2 or SEQ ID NO: 4 with one or mutations selected from L5F, T95I, D253G, E484K, D614G, and A701V, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from a Lambda SARS-CoV-2 strain, wherein the SARS-CoV-2 S protein or antigenic fragment thereof comprises the amino acid sequence of SEQ ID NO: 2 or SEQ ID NO: 4 with one or mutations selected from G75V, T76I, AR246-G252, D253N, L452Q, F490S, D614G, and T859N, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from a Mu SARS-CoV-2 strain, wherein the SARS-CoV-2 S protein or antigenic fragment thereof comprises the amino acid sequence of SEQ ID NO: 2 or SEQ ID NO: 4 with one or mutations selected from T95I, Y144S, Y145N, R346K, E484K, N501Y, D614G, P681H, and D950N, wherein the amino acid locations correspond to SEQ ID NO: 2 or SEQ ID NO: 4. In some aspects, the SARS-CoV-2 S proteins or antigenic fragments thereof disclosed herein include a SARS-CoV-2 S protein or antigenic fragment thereof from an Epsilon SARS-CoV-2 strain, wherein the SARS-CoV-2 S protein or antigenic fragment thereof comprises the amino acid sequence of SEQ ID NO: 2 or SEQ ID NO: 4 with one or mutations selected from S131, W152C, L452R, and D614G, wherein the amino acid locations correspond to SEQ ID NO: 2 or SEQ ID NO: 4.

Certain aspects of the disclosure relate to administering compositions including (i) a multicistronic DNA plasmid vector comprising a DNA sequence of one or more SARS-CoV-2 viral antigens and optionally a DNA sequence of one or more immune modifier proteins; (ii) a delivery component, such as a synthetic non-viral DNA carrier (e.g., a cationic polymer, a poly-inosinic-polycytidylic acid, or a poloxamer); and (iii) an adjuvant comprising an aluminum or aluminum-salt based adjuvant, a stimulator of interferon genes (STING) agonist, or a combination thereof. Certain aspects of the disclosure relate to administering compositions including (i) a multicistronic DNA plasmid vector comprising a DNA sequence of one or more monkeypox viral antigens and optionally a DNA sequence of one or more immune modifier proteins; (ii) a delivery component, such as a synthetic non-viral DNA carrier (e.g., a cationic polymer, a poly-inosinic-polycytidylic acid, or a poloxamer); and (iii) an adjuvant comprising an aluminum or aluminum-salt based adjuvant, a stimulator of interferon genes (STING) agonist, or a combination thereof. In some aspects, the compositions further comprise one or more immunopotentiators that are capable of activating the innate immunity system. In some aspects, the immunopotentiators are selected from a non-coding DNA (e.g., concatamers of non-coding 5′-C-phosphate-G-3′ (CpG) dinucleotides), a non-coding RNA, a small molecule, or any combination thereof.

Certain aspects of the disclosure relate to administering compositions including (i) a multicistronic RNA vector comprising an RNA sequence of one or more SARS-CoV-2 viral antigens and optionally an RNA sequence of one or more immune modifier proteins; (ii) a delivery component, such as a synthetic non-viral RNA carrier/adjuvant (e.g., a cationic polymer, a poly-inosinic-polycytidylic acid, or a poloxamer); and (iii) an adjuvant comprising an aluminum or aluminum-salt based adjuvant, a stimulator of interferon genes (STING) agonist, or a combination thereof. Certain aspects of the disclosure relate to administering compositions including (i) a multicistronic RNA vector comprising an RNA sequence of one or more monkeypox viral antigens and optionally an RNA sequence of one or more immune modifier proteins; (ii) a delivery component, such as a synthetic non-viral RNA carrier/adjuvant (e.g., a cationic polymer, a poly-inosinic-polycytidylic acid, or a poloxamer); and (iii) an adjuvant comprising an aluminum or aluminum-salt based adjuvant, a stimulator of interferon genes (STING) agonist, or a combination thereof.

Some aspects relate to methods of eliciting humoral and/or cellular immune response against a SARS-CoV-2 challenge, a monkeypox challenge, or infection following in vivo administration of a multicistronic DNA plasmid vector, a multicistronic mRNA vector, or a composition comprising the same of the disclosure.

Certain aspects of the disclosure relate to administering a composition comprising a polynucleotide comprising: (a) an antigen nucleic acid (e.g., first antigen nucleic acid) which encodes a first pathogen protein or an antigenic fragment thereof; and (b) a nucleic acid encoding an immune modifier protein. In some aspects, the polynucleotide is multicistronic (e.g., a multicistronic DNA plasmid or a multicistronic messenger RNA (mRNA)). In some aspects, the polynucleotide is combined with a delivery component, such as a synthetic non-viral carrier. In some aspects, the polynucleotide is combined with an adjuvant an adjuvant comprising an aluminum or aluminum-salt based adjuvant, a stimulator of interferon genes (STING) agonist, or a combination thereof.

In some aspects, provided herein is a polynucleotide (e.g., expression vector) comprising: (a) an antigen nucleic acid (e.g., first antigen nucleic acid) which encodes a SARS-CoV-2 spike (S) protein or an antigenic fragment thereof, wherein the antigen nucleic acid (e.g., first antigen nucleic acid) is operably linked to a promoter (e.g., a first promoter); and (b) a nucleic acid encoding an immune modifier protein. In some aspects, provided herein is a polynucleotide (e.g., expression vector) comprising: (a) an antigen nucleic acid (e.g., first antigen nucleic acid) which encodes a monkeypox protein or an antigenic fragment thereof, wherein the antigen nucleic acid (e.g., first antigen nucleic acid) is operably linked to a promoter (e.g., a first promoter); and (b) a nucleic acid encoding an immune modifier protein. In some aspects, the polynucleotide is a DNA vector for expression of a single pathogen antigen. In some aspects, the polynucleotide is a multicistronic DNA vector for expression of two or more pathogen antigens (e.g., two, three, four, five, six, seven or eight pathogen antigens) and/or an immune modifier.

In some aspects, the SARS-CoV-2 S protein or antigenic fragment thereof is a SARS-CoV-2 S protein or antigenic fragment thereof from a SARS-CoV-2 strain selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); and a Zeta strain (e.g., strain P.2).

In some aspects, the SARS-CoV-2 S protein or antigenic fragment thereof is from an Alpha SARS-CoV-2 strain, wherein the SARS-CoV-2 S protein or antigenic fragment thereof comprises the amino acid sequence of SEQ ID NO: 2 or SEQ ID NO: 4 with one or mutations selected from AH69-V70, A144, E484K, N501Y, A570D, D614G, P681H, T716I, S982A, and D1118H wherein the amino acid locations correspond to SEQ ID NO: 2 or SEQ ID NO: 4. In some aspects, the SARS-CoV-2 S protein or antigenic fragment thereof is from a Beta SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, D80A, D215G, AL241-S243, K417N, E484K, N501Y, D614G, and A701V, wherein the amino acid locations correspond to SEQ ID NO: 2 or SEQ ID NO: 4. In some aspects, the SARS-CoV-2 S protein or antigenic fragment thereof is from a Gamma SARS-CoV-2 strain, wherein the SARS-CoV-2 S protein or antigenic fragment thereof comprises the amino acid sequence of SEQ ID NO: 2 or SEQ ID NO: 4 with one or mutations selected from L18F, T20N, P26S, D138Y, R190S, K417T, E484K, N501Y, D614G, H655Y, T1027I, and V1176F, wherein the amino acid locations correspond to SEQ ID NO: 2 or SEQ ID NO: 4. In some aspects, the SARS-CoV-2 S protein or antigenic fragment thereof is from a Delta SARS-CoV-2 strain, wherein the SARS-CoV-2 S protein or antigenic fragment thereof comprises the amino acid sequence of SEQ ID NO: 2 or SEQ ID NO: 4 with one or mutations selected from T19R, AD119-F120, AE156-F157, R158G, L452R, T478K, D614G, P681R, and D950N, wherein the amino acid locations correspond to SEQ ID NO: 2 or SEQ ID NO: 4. In some aspects, the SARS-CoV-2 S protein or antigenic fragment thereof is from a Kappa SARS-CoV-2 strain, wherein the SARS-CoV-2 S protein or antigenic fragment thereof comprises the amino acid sequence of SEQ ID NO: 2 or SEQ ID NO: 4 with one or mutations selected from E154K, L452R, E484Q, D614G, P681R, and Q1071H, wherein the amino acid locations correspond to SEQ ID NO: 2 or SEQ ID NO: 4. In some aspects, the SARS-CoV-2 S protein or antigenic fragment thereof is from an Eta SARS-CoV-2 strain, wherein the SARS-CoV-2 S protein or antigenic fragment thereof comprises the amino acid sequence of SEQ ID NO: 2 or SEQ ID NO: 4 with one or mutations selected from Q52R, A67V, AH69-V70, AY144, E484K, D614G, Q677H, and F888L, wherein the amino acid locations correspond to SEQ ID NO: 2 or SEQ ID NO: 4. In some aspects, the SARS-CoV-2 S protein or antigenic fragment thereof is from an Iota SARS-CoV-2 strain, wherein the SARS-CoV-2 S protein or antigenic fragment thereof comprises the amino acid sequence of SEQ ID NO: 2 or SEQ ID NO: 4 with one or mutations selected from L5F, T95I, D253G, E484K, D614G, and A701V, wherein the amino acid locations correspond to SEQ ID NO: 2 or SEQ ID NO: 4. In some aspects, the SARS-CoV-2 S protein or antigenic fragment thereof is from a Lambda SARS-CoV-2 strain, wherein the SARS-CoV-2 S protein or antigenic fragment thereof comprises the amino acid sequence of SEQ ID NO: 2 or SEQ ID NO: 4 with one or mutations selected from G75V, T76I, AR246-G252, D253N, L452Q, F490S, D614G, and T859N, wherein the amino acid locations correspond to SEQ ID NO: 2 or SEQ ID NO: 4. In some aspects, the SARS-CoV-2 S protein or antigenic fragment thereof is from a Mu SARS-CoV-2 strain, wherein the SARS-CoV-2 S protein or antigenic fragment thereof comprises the amino acid sequence of SEQ ID NO: 2 or SEQ ID NO: 4 with one or mutations selected from T95I, Y144S, Y145N, R346K, E484K, N501Y, D614G, P681H, and D950N, wherein the amino acid locations correspond to SEQ ID NO: 2 or SEQ ID NO: 4. In some aspects, the SARS-CoV-2 S protein or antigenic fragment thereof is from an Epsilon SARS-CoV-2 strain, wherein the SARS-CoV-2 S protein or antigenic fragment thereof comprises the amino acid sequence of SEQ ID NO: 2 or SEQ ID NO: 4 with one or mutations selected from S131, W152C, L452R, and D614G, wherein the amino acid locations correspond to SEQ ID NO: 2 or SEQ ID NO: 4.

In some aspects, the polynucleotide comprises two or more nucleic acids encoding an immune modifier protein. In some aspects, each of the nucleic acids encoding an immune modifier protein encodes a different immune modifier protein. In some aspects, the two or more of the nucleic acids encoding an immune modifier protein encode IL-12 p35 and IL-12 p40.

In some aspects, the polynucleotide further comprises: (c) at least one additional antigen nucleic acid (e.g., second antigen nucleic acid) which encodes a second pathogen protein or an antigenic fragment thereof. In some aspects, the second pathogen protein or antigenic fragment thereof is selected from the group consisting of: a second viral antigen protein, a second bacterial antigen protein or a second parasite antigen protein.

In some aspects, the polynucleotide further comprises: (b) or (c) at least one additional antigen nucleic acid (e.g., second antigen nucleic acid) which encodes a SARS-CoV-2 protein or an antigenic fragment thereof. In some aspects, the SARS-CoV-2 protein or antigenic fragment thereof is selected from the group consisting of: a SARS-CoV-2 membrane (M) protein or an antigenic fragment thereof, a SARS-CoV-2 envelope (E) protein or an antigenic fragment thereof, a SARS-CoV-2 nucleocapsid (N) protein or an antigenic fragment thereof, and any combination thereof. In some aspects, the antigen nucleic acid (e.g., first antigen nucleic acid) and the at least one additional antigen nucleic acid (e.g., second antigen nucleic acid) encode SARS-CoV-2 proteins or antigenic fragments thereof from different strains of SARS-CoV-2. In some aspects, the antigen nucleic acid (e.g., first antigen nucleic acid) and the at least one additional antigen nucleic acid (e.g., second antigen nucleic acid) encode different variants of the same SARS-CoV-2 protein or antigenic fragment thereof, wherein the different variants of the same SARS-CoV2 protein or antigenic fragment thereof are derived from different strains of SARS-CoV-2. In some aspects, the antigen nucleic acid (e.g., first antigen nucleic acid) and the at least one additional antigen nucleic acid (e.g., second antigen nucleic acid) encode SARS-CoV-2 S proteins or antigenic fragments thereof from different strains of SARS-CoV-2. In some aspects, the SARS-CoV-2 S proteins or antigenic fragments thereof from different strains of SARS-CoV-2 comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety. In some aspects, the different strains of SARS-CoV-2 are selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); an Omicron SARS-CoV-2 strain (e.g., strains B.1.1.529/BA.1 BA.2, BA.5, BA.2.75, BQ.1, XBB1.5, and 22E), a Zeta strain (e.g., strain P.2); and any combination thereof. In some aspects, the different strains of SARS-CoV-2 are selected from the group consisting of: B.1.1.7, Q.1, Q.2, Q.3, Q.4, Q.5, Q.6, Q.7, Q.8, B.1.351, B.1.351.2, B.1.351.3, B.1.617.2, B.1.1.529, BA.2, BA. 5, BA.2.75, BQ.1, XBB1.5, 22E, AY.1 sublineages, P.1, P.1.1, P.1.2, B.1.427, B.1.429, B.1.525, B.1.526, B.1.617.1, B.1.617.3, B.1.621, B.1.621.1, P.2, and any combination thereof.

In some aspects, the at least one additional antigen nucleic acid (e.g., second antigen nucleic acid) is operably linked to the first promoter through an internal ribosome entry site (IRES) sequence. In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

In some aspects, the polynucleotide comprises one or more second promoters. In some aspects, the at least one additional antigen nucleic acid (e.g., second antigen nucleic acid) is operably linked to the one or more second promoters. In some aspects, one or more nucleic acids encoding an immune modifier protein is operably linked to the one or more second promoters. In some aspects, one or more of the nucleic acids encoding an immune modifier protein is operably linked to the first promoter or the one or more second promoters through an internal ribosome entry site (IRES) sequence. In some aspects, the IRES sequence comprises a nucleic acid sequence having at least 70%, at least 75%, at least 80%, at least 85%, at least 90%, at least 95%, at least 96%, at least 97%, at least 98%, at least 99%, or 100% sequence identity to SEQ ID NO: 41.

In some aspects, the first promoter or the one or more second promoters is selected from the group consisting of: a cytomegalovirus (CMV) promoter (SEQ ID NO: 31), a Rouse sarcoma virus (RSV) promoter, a Moloney murine leukemia virus (Mo-MuLV) long terminal repeat (LTR) promoter, a human ubiquitin C promoter, a mammalian elongation factor 1 (EF1) promoter, a human elongation factor 1α/Human T cell Leukemia Virus Type 1 Long Terminal Repeat (hEF1/HTLV) promoter, a cytokeratin 18 (CK18) promoter, a cytokeratin 19 (CK19) promoter, a simian virus 40 (SV40) promoter (SEQ ID NO: 32), a murine U6 promoter, a skeletal α-actin promoter, a β-actin promoter, a murine phosphoglycerate kinase 1 (PGK1) promoter, a human PGK1 promoter, a CBA promoter, a CAG promoter (SEQ ID NO: 33), and any combination thereof. In some aspects, the mammalian EF1 promoter is a hEF1-HTLV promoter (SEQ ID NO: 38). In some aspects, the one or more second promoters is the CMV promoter.

In some aspects, each of the nucleic acids which encodes an immune modifier protein is under the control of a promoter selected from the group consisting of a CMV promoter, an RSV promoter, a Mo-MuLV LTR promoter, a mammalian EF 1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CBA promoter, a CAG promoter, and any combination thereof. In some aspects, the mammalian EF 1 promoter is a hEF1-HTLV promoter.

In some aspects, each of the at least one additional antigen nucleic acid (e.g., second antigen nucleic acid)s is under the control of a promoter selected from the group consisting of a CMV promoter, an RSV promoter, a Mo-MuLV LTR promoter, a mammalian EF1 promoter, a CK18 promoter, a CK19 promoter, an SV40 promoter, a murine U6 promoter, a skeletal α-actin promoter, a β-actin promoter, a murine PGK1 promoter, a human PGK1 promoter, a CBA promoter, a CAG promoter, and any combination thereof. In some aspects, the mammalian EF1 promoter is a hEF1-HTLV promoter.

In some aspects, the immune modifier protein is selected from the group consisting of: interleukin (IL) 2 (IL-2), IL-12 p35, IL-12 p40, IL-12 p70, IL-15, IL-18, tumor necrosis factor alpha (TNFα), granulocyte-macrophage colony-stimulating factor (GM-CSF), interferon (IFN) a (IFN-α), IFN-β, a chemokine, major histocompatibility complex (MHC) class I (MHC I), MHC class II (MHC II), human leukocyte antigen (HLA)-DR isotype (HLA-DR), CD80, CD86, and any combination thereof. In some aspects, the chemokine is selected from the group consisting of: C—C motif chemokine ligand (CCL) 3 (CCL3), CCL4, CCL5, CCL21, CCL28, C—X—C motif chemokine ligand (CXCL) 10 (CXCL 10), and any combination thereof.

In some aspects, the immune modifier protein comprises a viral protein (e.g., SARS-CoV-2 non-structural protein 1 (Nsp 1), SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b) that attenuates a local inflammatory response and/or interferon response. In some aspects, the viral protein is from the same virus as a viral antigen encoded by an antigen nucleic acid. In some aspects, In some aspects, the viral protein is from a different virus than a viral antigen encoded by an antigen nucleic acid. In some aspects, the viral protein attenuates a local inflammatory response and/or interferon response elicited by a pathogen antigen disclosed herein. In some aspects, the immune modifier protein comprises SARS-CoV-2 Nsp1, SARS-CoV-2 Nsp6, SARS-CoV-2 Nsp13, SARS-CoV-2 ORF3a, SARS-CoV-2 ORF6, SARS-CoV-2 ORF7a, SARS-CoV-2 ORF7b, or any combination thereof.

In some aspects, the immune modifier protein comprises one or more concatamers of non-coding 5′-C-phosphate-G-3′ (CpG) dinucleotides. In some aspects, the one or more concatamers of non-coding CpG dinucleotides activate the Toll-like receptor 9 (TLR9) signaling pathway. In some aspects, the one or more concatamers of non-coding CpG dinucleotides comprise one or more concatamers of non-coding CpG dinucleotides previously reported in Bauer, A. et al., Nucleic Acids Research 38(12):3891-908 (2010); Cornelie, S. et al., Journal of Biological Chemistry 279(15):15124-9 (2004); Klinman, D. et al., J Immunol. 158(8):3635-9 (1997); Klinman, D. et al., Immunological Reviews 199(1):201-16 (2004); Luo, Z. et al., Mol Med Rep. 6(6):1309-14 (2012); Bode, C. et al., Expert Rev Vaccines 10(4):499-511 (2011); and Kuo, T. et al., Scientific Reports 10:20085 (2020), each of which is incorporated by reference herein in its entirety.

In some aspects, the nucleic acid encoding an immune modifier protein comprises a combination (i) a nucleic acid encoding an interleukin, and (ii) a nucleic acid encoding a major histocompatibility complex and/or a chemokine.

In some aspects, the nucleic acid encoding an immune modifier protein comprises a nucleic acid encoding IL-12, a nucleic acid encoding IL-15, or the combination thereof.

In some aspects, the nucleic acid encoding an immune modifier protein comprises a combination of a nucleic acid encoding IL-12 and a nucleic acid encoding IL-15.

In some aspects, the nucleic acid encoding an immune modifier protein comprises a nucleic acid encoding IL-2, a nucleic acid encoding IL-15, a nucleic acid encoding MHC I, a nucleic acid encoding MHC II, a nucleic acid encoding CCL3, a nucleic acid encoding CCL4, or any combination thereof.

In some aspects, the nucleic acid encoding an immune modifier protein comprises a nucleic acid encoding MHC I, a nucleic acid encoding MHC II, a nucleic acid encoding CCL3, a nucleic acid encoding CCL4, or any combination thereof.

In some aspects, the nucleic acid encoding an immune modifier protein comprises a nucleic acid encoding CCL3, a nucleic acid encoding CCL4, or the combination thereof.

In some aspects, the polynucleotides or nucleic acids can comprise DNA or mRNA sequences.

In some aspects, the polynucleotide further comprises one or more post-transcriptional regulatory elements. In some aspects, the post-transcriptional regulatory element is a wood chuck hepatitis virus post-transcriptional regulatory element (WPRE).

In some aspects, the polynucleotide further comprises at least one 3′ UTR poly(a) tail sequence operably linked to the antigen nucleic acid (e.g., first antigen nucleic acid), the at least one additional antigen nucleic acid (e.g., second antigen nucleic acid), the nucleic acid encoding an immune modifier protein, or any combination thereof. In some aspects, the 3′ UTR poly(a) tail sequence is a 3′ UTR SV40 poly(a) tail sequence (SEQ ID NO: 29), a 3′ UTR bovine growth hormone (bGH) poly(A) sequence (SEQ ID NO: 30), a 3′ UTR actin poly(A) tail sequence, a 3′ UTR hemoglobin poly(A) sequence, or any combinations thereof.

In some aspects, the polynucleotide further comprises at least one enhancer sequence. In some aspects, the enhancer sequence is a human actin enhancer sequence, a human myosin enhancer sequence, a human hemoglobin enhancer sequence, a human muscle creatine enhancer sequence, a viral enhancer sequence, or a polynucleotide function enhancer sequence. In some aspects, the enhancer sequence is a CMV intronic sequence or a β-actin intronic sequence. In aspects, the enhancer sequence is a SV40 enhancer sequence (SEQ ID NO: 37). In some aspects, the enhancer sequence is a CMV intron A sequence (SEQ ID NO: 128). In some aspects, the polynucleotide comprises both a SV40 enhancer sequence (SEQ ID NO: 37) and a CMV intron A sequence (SEQ ID NO: 128).

In some aspects, the polynucleotide comprises a leader sequence. In some aspects, the leader sequence is an IgE leader sequence. In some aspects, the leader sequence comprises a nucleic acid sequence having at least 70%, at least 75%, at least 80%, at least 85%, at least 90%, at least 95%, at least 99%, or 100% sequence identity to SEQ ID NO: 129. In some aspects, the leader sequence encodes a polypeptide having at least 70%, at least 75%, at least 80%, at least 85%, at least 90%, at least 95%, or at least 99% sequence identity to the amino acid sequence of SEQ ID NO: 130.

In some aspects, the polynucleotide is a multicistronic mRNA comprising a 5′ cap and a 3′ UTR poly(A) tail sequence. In some aspects, the 3′ UTR poly(a) tail sequence is a 3′ UTR SV40 poly(a) tail sequence (SEQ ID NO: 29), a 3′ UTR bovine growth hormone (bGH) poly(A) sequence (SEQ ID NO: 30), a 3′ UTR actin poly(A) tail sequence, a 3′ UTR hemoglobin poly(A) sequence, or any combinations thereof. In some aspects, the multicistronic mRNA comprises a 5′ UTR and/or a 3′ UTR.

Also provided herein is a vector comprising any polynucleotide described or exemplified herein, wherein the vector is a DNA plasmid, a multicistronic mRNA, a viral vector, a bacterial vector, a cosmid, or an artificial chromosome.

In some aspects, the composition, pharmaceutical composition, or vaccine further comprises a second polynucleotide encoding at least one immune modifier protein selected from the group consisting of: IL-2, IL-12 p35, IL-12 p40, IL-12 p70, IL-15, IL-18, TNFα, GM-CSF, IFN-α, IFN-β, a chemokine, MHC I, MHC II, HLA-DR, CD80, and CD86, wherein the polynucleotide encoding the at least one immune modifier protein is operably linked to a promoter. In some aspects, the composition, pharmaceutical composition, or vaccine further comprises a delivery component.

Also provided herein is a vector (e.g., a plasmid vector) comprising any polynucleotide (e.g., DNA or mRNA) described or exemplified herein. In some aspects, the plasmid vector is a DNA plasmid vector.

Also provided herein is a composition, pharmaceutical composition, or vaccine comprising any polynucleotide, vector, multicistronic mRNA vector, or DNA plasmid vector described or exemplified herein.

Also provided herein are methods of administering a composition, pharmaceutical composition, or vaccine comprising: (a) a polynucleotide or a vector comprising the polynucleotide, and (b) a delivery component, wherein the polynucleotide comprises a antigen nucleic acid (e.g., first antigen nucleic acid) which encodes a first pathogen protein or an antigenic fragment thereof, wherein the antigen nucleic acid (e.g., first antigen nucleic acid) is operably linked to a first promoter, optionally wherein the delivery component is a cationic polymer, a poly-inosinic-polycytidylic acid, or a poloxamer or derivative thereof. In some aspects, the delivery component is a cationic polymer, a poly-inosinic-polycytidylic acid, or a poloxamer or derivative thereof. In some aspects, the antigen nucleic acid (e.g., first antigen nucleic acid) which encodes a first pathogen protein is selected from the group consisting of a viral protein, a bacterial protein, a parasite protein, and any antigenic fragment thereof. In some aspects, the delivery component further comprises benzalkonium chloride.

The delivery component of the compositions, pharmaceutical compositions, or vaccines may comprise any combination of delivery components disclosed herein.

In some aspects, the polynucleotide further comprises at least one additional antigen nucleic acid (e.g., second antigen nucleic acid) which encodes a second pathogen protein or an antigenic fragment thereof. In some aspects, the at least one additional antigen nucleic acid (e.g., second antigen nucleic acid) which encodes a second pathogen protein is selected from the group consisting of a viral protein, a bacterial protein, a parasite protein, and any antigenic fragment thereof.

In some aspects, the first pathogen protein and/or the second pathogen protein is/are selected from the group consisting of a Yersinia pestis antigen, a Mycobacterium tuberculosis antigen, a Meningococcus antigen, a Rabies virus antigen, a Cytomegalovirus antigen, a Yellow fever virus antigen, an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a orthopoxvirus antigen, a monkeypox antigen, a vaccinia antigen, a smallpox antigen, a Epstein bar virus antigen, a nipha virus antigen, a varicella-zoster virus antigen, a Clostridioides difficile antigen, a Streptococcus pneumonia antigen, a Neisseria meningitides antigen, a Toxoplasma gondii antigen, a Plasmodium falciparum antigen, an influenza virus antigen, antigenic fragments thereof, and any combinations thereof.

In some aspects, the first pathogen protein and/or the second pathogen protein is/are selected from the group consisting of: a Yersinia pestis F1-Ag, a Yersinia pestis V-Ag, a Mycobacterium tuberculosis Apa antigen, a Mycobacterium tuberculosis HP65 antigen, a Mycobacterium tuberculosis rAg85A antigen, an E71 VP1 antigen, a GST-tagged E71-VP1 antigen, a Cox protein antigen, a GST-tagged Cox protein antigen, an HSV-1 envelope antigen, an HSV-2 envelope antigen, an HSV-2 gB2 antigen, an HSV-2 gC2 antigen, an HSV-2 gD2 antigen, an HSV-2 gE2 antigen, an HIV Env antigen, an HIV Gag antigen, an HIV Nef antigen, an HIV Pol antigen, an HPV minor capsid protein L2 antigen, a human papillomavirus type 16 Regulatory protein E2 antigen, a human papillomavirus type 16 Protein E6 antigen, a human papillomavirus type 16 Protein E7 antigen, a human papillomavirus type 18 Regulatory protein E2 antigen, a human papillomavirus type 18 Protein E6 antigen, a human papillomavirus type 18 Protein E7 antigen, a human papillomavirus type 6a Regulatory protein E2 antigen, a human papillomavirus type 6a Protein E6 antigen, a human papillomavirus type 6a Protein E7 antigen, a human papillomavirus 11 Regulatory protein E2 antigen, a human papillomavirus 11 Protein E6 antigen, a human papillomavirus 11 Protein E7 antigen, an HCV NS3 antigen, a hepatitis C virus genotype 1a Genome polyprotein antigen, a hepatitis C virus genotype 1b Genome polyprotein antigen, a hepatitis C virus genotype 2a Genome polyprotein antigen, a hepatitis C virus genotype 3a Genome polyprotein antigen, a RSV F antigen, a RSV G antigen, a Dengue virus E protein antigen, a Dengue virus EDIII antigen, a Dengue virus NS1 antigen, a Dengue virus DEN-80E antigen, an Ebola virus GB antigen, an Ebola virus VP24 antigen, an Ebola virus VP40 antigen, an Ebola virus NP antigen, an Ebola virus VP30 antigen, an Ebola virus VP35 antigen, a Zika virus envelope domain III antigen, a Zika virus CKD antigen, a Chikungunya virus E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), a measles virus hemagglutinin protein MV-H antigen, a measles virus fusion protein MV-F antigen, a MERS-CoV S protein antigen, an antigen from the receptor-binding domain of the MERS-CoV S protein, an antigen from the membrane fusion domain of the MERS-CoV S protein, a SARS-CoV S protein antigen, an antigen from the receptor binding domain of the SARS-CoV S protein, an antigen from the membrane fusion domain of the SARS-CoV S protein, a SARS-CoV E protein antigen, a SARS-CoV M protein antigen, a SARS-CoV N protein, a monkeypox A35R protein antigen, a monkeypox H3L protein antigen, a monkeypox L1R protein antigen, a Clostridioides difficile 630 spore coat protein: peroxiredoxin/chitinase antigen, a Clostridioides difficile 630 flagellin C antigen, a Clostridioides difficile Surface layer protein A (Fragment) antigen, an Epstein-Barr virus (strain B95-8) nuclear antigen 1 antigen, an Epstein-Barr virus (strain B95-8) Envelope glycoprotein B antigen, an Epstein-Barr virus (strain B95-8) Envelope glycoprotein H antigen, an Epstein-Barr virus (strain B95-8) Envelope glycoprotein GP350 antigen, an Epstein-Barr virus (strain B95-8) Latent membrane protein 1 antigen, an Epstein-Barr virus (strain B95-8) Latent membrane protein 2 antigen, a Neisseria meningitides Factor H-binding protein antigen, a Neisseria meningitidis serogroup B Neisseria adhesin A antigen, a Neisseria meningitidis Neisserial heparin binding antigen antigen, a Vaccinia virus (strain Western Reserve) Protein A27 antigen, a Vaccinia virus (strain Western Reserve) EEV membrane phosphoglycoprotein antigen, a Vaccinia virus B5R (Fragment) antigen, a Vaccinia virus Envelope protein H3 antigen, a Vaccinia virus (strain Western Reserve) IMV membrane protein antigen, a Nipah virus Fusion glycoprotein FO antigen, a Nipah virus Glycoprotein G antigen, a Varicella-zoster virus (strain Dumas) Envelope glycoprotein E antigen, a Toxoplasma gondii MIC8 antigen, a Plasmodium falciparum SERA5 polypeptide antigen, a Plasmodium falciparum circumsporozite protein antigen, an influenza virus hemagglutinin (HA) antigen, an influenza virus neuraminidase (NA) antigen, an influenza virus matrix-1 (M1) protein antigen, antigen an influenza virus matrix-2 (M2) protein antigen, an influenza virus matrix-2 (M2) protein antigen, an influenza RNA polymerase subunit PB1 antigen, an influenza RNA polymerase subunit PB2 antigen, an influenza RNA polymerase subunit PA antigen, an influenza non-structural protein 1 (NS1) antigen, an influenza non-structural protein 2 (NS2) protein antigenic fragments thereof, and any combination thereof.

In some aspects, the delivery component of the composition, pharmaceutical composition, or vaccine is a cationic polymer. In some aspects, the cationic polymer is a synthetic functionalized polymer, a lipid, a lipopolymer, or a chemical derivative thereof. In some aspects, the synthetic functionalized polymer is a biodegradable cross-linked cationic multi-block copolymer.

In some aspects, the biodegradable cross-linked cationic multi-block copolymer is represented by the formula: (CP)xLyYz, wherein: (a) CP represents a cationic polymer containing at least one secondary amine group, wherein the cationic polymer has a number averaged molecular weight within the range of 1,000 to 25,000 Dalton; (b) Y represents a bifunctional biodegradable linker containing ester, amide, disulfide, or phosphate linages; (c) L represents a ligand; (d) x is an integer in the range from 1 to 20; (e) y is an integer in the range from 0 to 100; and (f) z is an integer in the range from 0 to 40. In some aspects, the cationic polymer comprises linear polyethyleneimine (LPEI). In some aspects, the cationic polymer comprising linear polyethyleneimine (LPEI) is BD15K-12, wherein the PEIs are approximately 15,000 Da, and wherein there is an average of 12 crosslinkers per PEI. In some aspects, the bifunctional biodegradable linker is hydrophilic and comprises a biodegradable linkage comprising a disulfide bond. In some aspects, the bifunctional biodegradable linker is a dithiodipropionyl linker.

In some aspects, the biodegradable cross-linked cationic multi-block copolymer comprises LPEI and a dithiodipropionyl linker for cross-linking the multi-block copolymer, wherein the LPEI has an average molecular weight of 1,000 to 25,000 Dalton. In some aspects, the biodegradable cross-linked cationic multi-block copolymer is covalently linked to at least one ligand.

In some aspects, the ligand is a targeting ligand selected from the group consisting of: a sugar moiety, a polypeptide, folate, and an antigen. In some aspects, the sugar moiety is a monosaccharide. In some aspects, the monosaccharide is galactose. In some aspects, the sugar moiety is an oligosaccharide. In some aspects, the polypeptide is a glycoprotein, an antibody, an antibody fragment, a cell receptor, a cytokine receptor, or a growth factor receptor. In some aspects, the growth factor receptor is an epidermal growth factor receptor. In some aspects, the glycoprotein is transferrin or asialoorosomucoid (ASOR). In some aspects, the antigen is a viral antigen, a bacterial antigen, or a parasite antigen.

In some aspects, the biodegradable cross-linked cationic multi-block copolymer is covalently linked to polyethylene glycol (PEG) of molecular weight ranging from 500 to 20,000 Dalton. In some aspects, the biodegradable cross-linked cationic multi-block copolymer is covalently linked to a fatty acyl chain selected from the group consisting of: oleic acid, palmitic acid, and stearic acid. In some aspects, the biodegradable cross-linked cationic multi-block copolymer comprises at least one amine group that is electrostatically attracted to a polyanionic compound. In some aspects, the polyanionic compound is a nucleic acid, wherein the biodegradable cross-linked cationic multi-block copolymer condenses the nucleic acid to form a compact structure.

In some aspects, the delivery component of the composition, pharmaceutical composition, or vaccine is a cationic lipopolymer comprising a PEI backbone covalently linked to a lipid or a PEG. In some aspects, the PEI backbone is covalently linked to a lipid and a PEG. In some aspects, the lipid and the PEG are directly attached to the PEI backbone by covalent bonds. In some aspects, the lipid is attached to the PEI backbone through a PEG spacer. In some aspects, the PEG has a molecular weight of between 50 to 20,000 Dalton. In some aspects, the molar ratio of PEG to PEI is within a range of 0.1:1 to 500:1. In some aspects, the molar ratio of the lipid to the PEI is within a range of 0.1:1 to 500:1. In some aspects, the lipid is a cholesterol, a cholesterol derivative, a C₁₂ to C₁₈ fatty acid, or a fatty acid derivative. In some aspects, the PEI is covalently linked to cholesterol and PEG, and wherein the average PEG:PEI:cholesterol molar ratio in the cationic lipopolymer is within the range of 1-5 PEG:1 PEI:0.4-1.5 cholesterol. In some aspects, the PEI has a linear or branch configuration with a molecular weight of 100 to 500,000 Dalton.

In some aspects, the cationic lipopolymer further comprises a pendant functional moiety selected from the group consisting of: a receptor ligand, a membrane permeating agent, an endosomolytic agent, a nuclear localization sequence, and a pH sensitive endosomolytic peptide.

In some aspects, the cationic lipopolymer further comprises a targeting ligand, wherein the targeting ligand is directly attached to the PEI backbone or is attached through a PEG linker. In some aspects, the targeting ligand is selected from the group consisting of: a sugar moiety, a polypeptide, folate, and an antigen. In some aspects, the sugar moiety is a monosaccharide. In some aspects, the monosaccharide is galactose. In some aspects, the sugar moiety is an oligosaccharide. In some aspects, the polypeptide is a glycoprotein, an antibody, an antibody fragment, a cell receptor, a cytokine receptor, or a growth factor receptor. In some aspects, the growth factor receptor is an epidermal growth factor receptor. In some aspects, the glycoprotein is transferrin or asialoorosomucoid (ASOR). In some aspects, the antigen is a viral antigen, a bacterial antigen, or a parasite antigen.

In some aspects, the cationic polymer of the composition, pharmaceutical composition, or vaccine is present in an amount sufficient to produce a ratio of amine nitrogen in the cationic polymer to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the cationic polymer to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the cationic polymer to phosphate in the polynucleotide, multicistronic mRNA vector, or DNA plasmid vector is from about 1:10 to about 10:1.

In some aspects, the composition, pharmaceutical composition, or vaccine comprises about 0.1 mg/ml to about 10 mg/ml (e.g., about 0.1 mg/ml to about 5 mg/ml; about 0.5 mg/ml to about 10 mg/ml; or about 0.5 mg/ml to about 5 mg/ml) nucleic acid (e.g., DNA plasmid vector or multicistronic mRNA vector) complexed with the cationic polymer. In some aspects, the composition, pharmaceutical composition, or vaccine comprises about 1 mg/ml to about 10 mg/ml (e.g., about 1 mg/ml to about 6 mg/ml; about 2 mg/ml to about 6 mg/ml; about 5 mg/ml to about 10 mg/ml; or about 6 mg/ml to about 10 mg/ml) nucleic acid (e.g., DNA plasmid vector or multicistronic mRNA vector) complexed with the cationic polymer.

In some aspects, the delivery component of the composition, pharmaceutical composition, or vaccine comprises a lipopolyamine with the following formula:

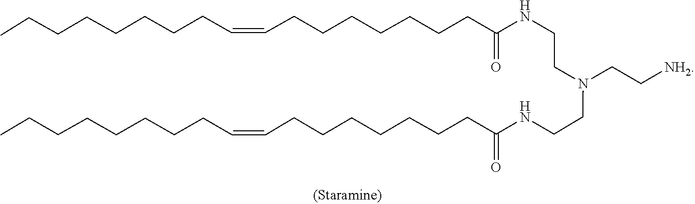 [SMILES: CCCCCCCC/C=C\CCCCCCCC(=O)NCCN(CCN)CCNC(=O)CCCCCCC/C=C\CCCCCCCC]

In some aspects, the delivery component comprises a mixture of the lipopolyamine and an alkylated derivative of the lipopolyamine. In some aspects, the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride. In some aspects, the alkylated derivative of the lipopolyamine has the following formula:

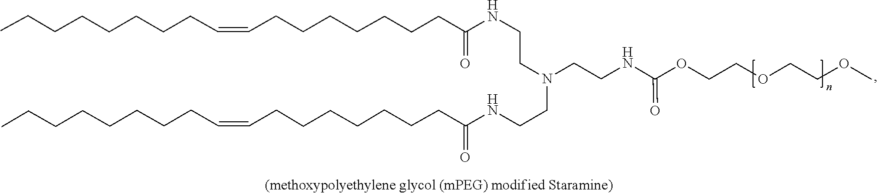

wherein n represents an integer from 10 to 100 repeating units containing 2-5 carbon atoms each. In some aspects, the alkylated derivative of the lipopolyamine has the following formula:

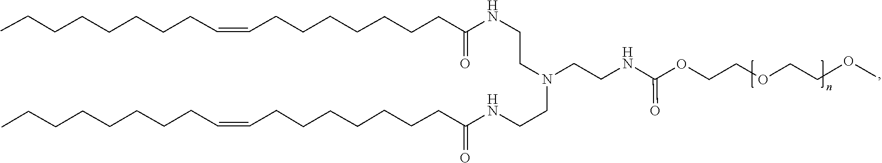

wherein n=11 (Staramine-mPEG515). In some aspects, the alkylated derivative of the lipopolyamine has the following formula:

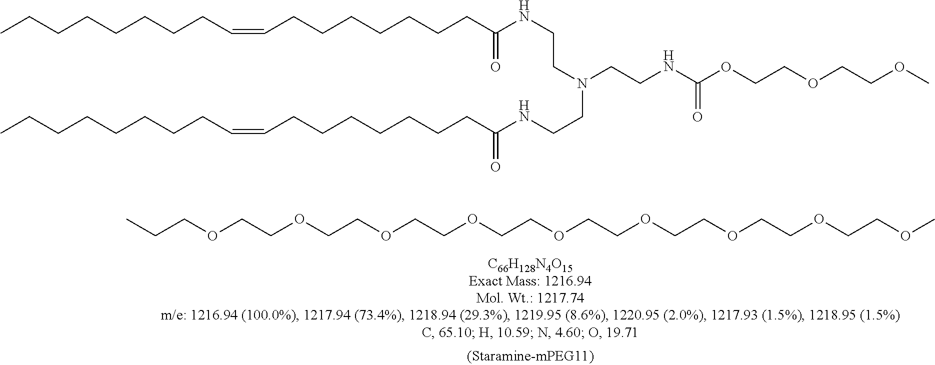

In some aspects, the ratio of the lipopolyamine to the alkylated derivative of the lipopolyamine in the mixture is 0.01:1 to 10:1. In some aspects, the lipopolyamine is present in an amount sufficient to produce a ratio of amine nitrogen in the lipopolyamine to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the lipopolyamine to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the lipopolyamine to phosphate in the polynucleotide, multicistronic mRNA vector, or DNA plasmid vector is from about 0.01:10 to about 10:1.

In some aspects, the delivery component of the composition, pharmaceutical composition, or vaccine comprises a lipopolyamine with the following formula:

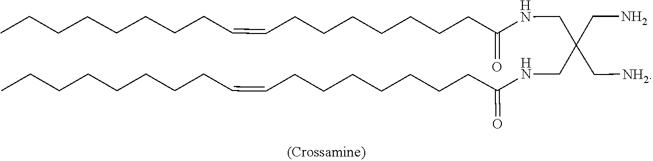 [SMILES: CCCCCCCC/C=C\CCCCCCCC(=O)NCC(CN)(CN)CNC(=O)CCCCCCC/C=C\CCCCCCCC]

In some aspects, the delivery component comprises a mixture of the lipopolyamine and an alkylated derivative of the lipopolyamine. In some aspects, the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride. In some aspects, the ratio of the lipopolyamine to the alkylated derivative of the lipopolyamine in the mixture is 0.01:1 to 10:1. In some aspects, the lipopolyamine is present in an amount sufficient to produce a ratio of amine nitrogen in the lipopolyamine to phosphate in the polynucleotide, multicistronic mRNA vector, or DNA plasmid vector from about 0.01:1 to about 50:1 (e.g., about 0.01:1 to about 40:1; about 0.01:1 to about 30:1; about 0.01:1 to about 20:1; about 0.01:1 to about 10:1, or about 0.01:1 to about 5:1). In some aspects, the ratio of amine nitrogen in the lipopolyamine to phosphate in the polynucleotide, multicistronic mRNA vector, or DNA plasmid vector is from about 0.1:1 to about 50:1 (e.g., about 0.1:1 to about 40:1; about 0.1:1 to about 30:1; about 0.1:1 to about 20:1; about 0.1:1 to about 10:1, or about 0.1:1 to about 5:1). In some aspects, the ratio of amine nitrogen in the lipopolyamine to phosphate in the polynucleotide, multicistronic mRNA vector, or DNA plasmid vector is from about 1:10 to about 10:1.

In some aspects, the delivery component of the composition, pharmaceutical composition, or vaccine comprises a poloxamer back-bone having a metal chelator covalently coupled to at least one terminal end of the poloxamer backbone. In some aspects, the metal chelator is coupled to at least two terminal ends of the poloxamer backbone. In some aspects, at least one metal chelator is coupled to a poloxamer. In some aspects, at least two metal chelators are coupled to a poloxamer. In some aspects, 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, or 95-100 metal chelators are coupled to a poloxamer.

In some aspects, the poloxamer backbone is a poloxamer backbone disclosed in U.S. Publ. No. 2010/0004313, which is herein incorporated by reference in its entirety. In some aspects, the metal chelator is a metal chelator disclosed in U.S. Publ. No. 2010/0004313. In some aspects, the delivery component of the composition, pharmaceutical composition, or vaccine comprises a polymer having the following formula:

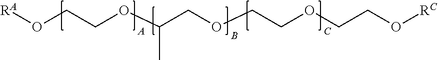

and pharmaceutically acceptable salts thereof, wherein:
A represents an integer from 2 to 141;
B represents an integer from 16 to 67;
C represents an integer from 2 to 141;
RA and RC are the same or different, and are R′-L- or H, wherein at least one of RA and RC is R′-L-;
L is a bond, —CO—, —CH₂—O—, or —O—CO—; and
R′ is a metal chelator.

In some aspects, the delivery component comprises a poloxamer with the following formula:

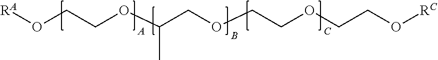

or a pharmaceutically acceptable salt thereof, wherein:
A represents an integer from 2 to 141;
B represents an integer from 16 to 67;
C represents an integer from 2 to 141;
RA and RC are the same or different, and are R′-L- or H;
L is a bond, —CO—, —CH₂—O—, or —O—CO—; and
R′ is a metal chelator.

In some aspects, at least one of RA and RC is R′-L-.

In some aspects, the R′ is covalently bound to the poloxamer (e.g., where a metal chelator (e.g., a crown) is covalently attached at different density to the poloxamer).

In some aspects, one metal chelator or two or more metal chelators is/are bound to the poloxamer.

In some aspects, 2-100 metal chelators are bound to the poloxamer.

In some aspects, the metal chelator is RNNH—, RN2N—, or (R″—(N(R″)—CH2CH2)x)2-N—CH2CO—, wherein each x is independently 0-2, and wherein R″ is HO₂C—CH2-. In some aspects, the metal chelator is a crown ether selected from the group consisting of 12-crown-4, 15-crown-5, 18-crown-6, 20-crown-6, 21-crown-7, and 24-crown-8. In some aspects, the crown ether is a substituted-crown ether, wherein the substituted crown ether has:
(1) one or more of the crown ether oxygens independently replaced by NH or S,
(2) one or more of the crown ether —CH2-CH2- moieties replaced by C6H4-, —C10H6-, or —C6H10-,
(3) one or more of the crown ether —CH2-O—CH2- moieties replaced by C4H2O— or —C5H3N—, or
(4) any combination thereof.

In some aspects, the metal chelator is a cryptand, wherein the cryptand is selected from the group consisting of (1,2,2) cryptand, (2,2,2) cryptand, (2,2,3) cryptand, and (2,3,3) cryptand. In some aspects, the cryptand is a substituted-cryptand, wherein the substituted cryptand has:
(1) one or more of the crypthand ether oxygens independently replaced by NH or S,
(2) one or more of the crown ether —CH2-CH2- moieties replaced by C6H4-, —C10H6-, or —C6H10-,
(3) one or more of the crown ether —CH2-O—CH2- moieties replaced by C4H2O— or —C5H3N—, or
(4) any combination thereof.

In some aspects, the delivery component is Crown Poloxamer (aza-crown-linked poloxamer), wherein the Crown Poloxamer comprises a polymer having the following formula:

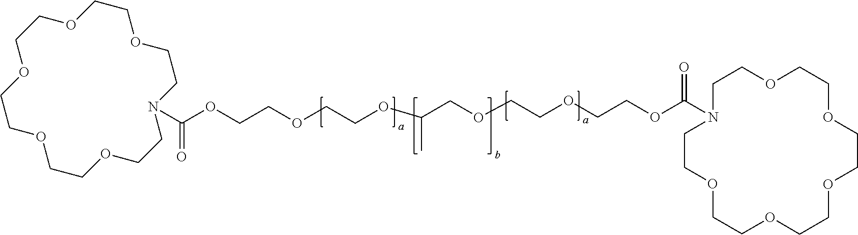

or pharmaceutically acceptable salts thereof, wherein:
a represents an integer of about 10 units; and
b represents an integer of about 21 units; and
wherein the total molecular weight of the polymer is about 2,000 Da to about 2,200 Da.

In some aspects, the crown poloxamer can be derivatized with a cationic molecule, a ligand, or other chemical entities.

In some aspects, the polymer is present in a solution with the polynucleotide, multicistronic mRNA vector, or DNA plasmid vector from about 0.1%-about 5% or about 0.5%-about 5%.

In some aspects, the delivery component of the composition, pharmaceutical composition, or vaccine is a 3-amino ester.

In some aspects, the polymer or poloxamer (e.g., crown poloxamer) is present in a solution with the polynucleotide, multicistronic mRNA vector, or DNA plasmid vector from about 0.1%-about 5% or about 0.5%-about 5%.

In some aspects, the solution is co-formulated with a metal chelator (e.g., where the co-formulated metal chelator is a free metal chelator, which is formulated with the poloxamer (e.g., a non-crown poloxamer)).

In some aspects, the co-formulated metal chelator is present in the solution at a concentration of about 0.1 mg/mL to about 20 mg/mL.

In some aspects, the co-formulated metal chelator is crown ether, a substituted-crown ether, a cryptand, or a substituted-cryptand.

In some aspects, the metal chelator or co-formulated metal chelator is crown ether (Aza-18-crown-6).

In some aspects, the delivery component of the composition, pharmaceutical composition, or vaccine is a poly-inosinic-polycytidylic acid. In some aspects, the poly-inosinic-polycytidylic acid is present in a solution with the polynucleotide, multicistronic mRNA vector, or DNA plasmid vector from about 0.1%-about 5% or about 0.5%-about 5%.

In some aspects, the delivery component further comprises benzalkonium chloride.

In some aspects, the delivery component comprises BD15-12. In some aspects, the ratio of nucleotide to BD15-12 polymer (N:P) is 0.01:1 to 5:1.

In some aspects, the delivery component comprises Omnifect. In some aspects, the ratio of nucleotide to Omnifect polymer (N:P) is 0.01:1 to 5:1.

In some aspects, the delivery component comprises Crown Poloxamer (aza-crown-linked poloxamer). In some aspects, the ratio of nucleotide to Crown Poloxamer (N:P) is 0.01:1 to 5:1. In some aspects, the delivery component comprises Crown Poloxamer and a PEG-PEI-cholesterol (PPC) lipopolymer. In some aspects, the delivery component comprises Crown Poloxamer and benzalkonium chloride. In some aspects, the delivery component comprises Crown Poloxamer and Omnifect. In some aspects, the delivery component comprises Crown Poloxamer and a linear polyethyleneimine (LPEI). In some aspects, the delivery component comprises Crown Poloxamer and BD15-12.

In some aspects, the delivery component comprises Staramine and mPEG modified Staramine. In some aspects, the mPEG modified Staramine is Staramine-mPEG515. In some aspects, the mPEG modified Staramine is Staramine-mPEG11. In some aspects, the ratio of Staramine to mPEG modified Staramine is 0.01:1 to 10:1. In some aspects, the nucleotide to polymer (N:P) ratio is 0.01:1 to 5:1. In some aspects, the delivery component comprises Staramine, mPEG modified Staramine, and Crown Poloxamer. In some aspects, the delivery component comprises Staramine, Staramine-mPEG515, and Crown Poloxamer. In some aspects, the delivery component comprises Staramine, Staramine-mPEG11, and Crown Poloxamer.

In some aspects, the metal chelator is added directly to a solution comprising (i) the polynucleotide, multicistronic mRNA vector, or DNA plasmid vector as described herein, and (ii) a poloxamer. In some aspects, the metal chelator is present in the solution at concentration of about 0.1 mg/mL to about 20 mg/mL. In some aspects, metal chelator is present in the solution at concentration of about 0.1 mg/mL to about 20 mg/mL, about 0.1 mg/mL to about 1 mg/mL, about 1 mg/mL to about 5 mg/mL, about 5 mg/mL to about 10 mg/mL, about 10 mg/mL to about 15 mg/mL, or about 15 mg/mL to about 20 mg/mL. In some aspects, the metal chelator is crown ether, a substituted-crown ether, a cryptand, or a substituted-cryptand. In some aspects, the metal chelator is crown ether (Aza-18-crown-6).

In some aspects, the metal chelator is added directly to a solution comprising (i) the polynucleotide, multicistronic mRNA vector, or DNA plasmid vector as described herein, (ii) a poloxamer, and (iii) an an adjuvant comprising an aluminum or aluminum-salt based adjuvant, a stimulator of interferon genes (STING) agonist, or a combination thereof. In some aspects, the metal chelator is present in the solution at concentration of about 0.1 mg/mL to about 20 mg/mL. In some aspects, metal chelator is present in the solution at concentration of about 0.1 mg/mL to about 20 mg/mL, about 0.1 mg/mL to about 1 mg/mL, about 1 mg/mL to about 5 mg/mL, about 5 mg/mL to about 10 mg/mL, about 10 mg/mL to about 15 mg/mL, or about 15 mg/mL to about 20 mg/mL. In some aspects, the metal chelator is crown ether, a substituted-crown ether, a cryptand, or a substituted-cryptand. In some aspects, the metal chelator is crown ether (Aza-18-crown-6). In some aspects, the adjuvant is AIPO₄.

In some aspects, the metal chelator is added directly to a solution comprising (i) the polynucleotide, multicistronic mRNA vector, or DNA plasmid vector as described herein, (ii) a poloxamer, and (iii) an an adjuvant comprising an aluminum or aluminum-salt based adjuvant, or a combination thereof. In some aspects, the metal chelator is present in the solution at concentration of about 0.1 mg/mL to about 20 mg/mL. In some aspects, metal chelator is present in the solution at concentration of about 0.1 mg/mL to about 20 mg/mL, about 0.1 mg/mL to about 1 mg/mL, about 1 mg/mL to about 5 mg/mL, about 5 mg/mL to about 10 mg/mL, about 10 mg/mL to about 15 mg/mL, or about 15 mg/mL to about 20 mg/mL. In some aspects, the metal chelator is crown ether, a substituted-crown ether, a cryptand, or a substituted-cryptand. In some aspects, the metal chelator is crown ether (Aza-18-crown-6). n some aspects, the adjuvant is AIPO₄.

In some aspects, the composition, pharmaceutical composition, or vaccine is lyophilized.

Also provided herein is a host cell comprising any polynucleotide, vector, multicistronic mRNA vector, DNA plasmid vector, composition, pharmaceutical composition, or vaccine described or exemplified herein. In some aspects, the host cell is a eukaryotic host cell. In some aspects, the host cell is a human host cell.

Also provided herein is a kit comprising any polynucleotide, vector, multicistronic mRNA vector, DNA plasmid vector, composition, pharmaceutical composition, vaccine, or lyophilized composition described or exemplified herein. In some aspects, the kit further comprises a glass vial. In some aspects, the kit further comprises instructions for using the polynucleotide, vector, multicistronic mRNA vector, DNA plasmid vector, composition, or pharmaceutical composition in a method for inducing an immune response in a subject. In some aspects, the kit further comprise instruction for reconstituting the composition, pharmaceutical composition, vaccine or lyophilized vaccine. In some aspects, the kit further comprises instructions for using the polynucleotide, vector, multicistronic mRNA vector, DNA plasmid vector, composition, pharmaceutical composition, vaccine, or lyophilized composition in a method for preventing, reducing the incidence of, attenuating or treating a virus (e.g., SARS-CoV-2 or monkeypox), a bacteria or a parasite infection in a subject.

Also provided herein is a method of inducing an immune response in a subject, the method comprising administering an effective amount of any polynucleotide, vector, multicistronic mRNA vector, DNA plasmid vector, composition, pharmaceutical composition, or vaccine described or exemplified herein to the subject. In some aspects, the immune response is to one or more antigens disclosed herein. In some aspects, the immune response is a protective immune response. In some aspects, the polynucleotide, vector, multicistronic mRNA vector, DNA plasmid vector, composition, pharmaceutical composition, or vaccine is administered to the subject by an intramuscular, transdermal, subcutaneous, intralymphatic, intranasal, or intraperitoneal route of administration.

In some aspects, the immune response is to one or more antigens comprising one or more viral antigens, one or more bacterial antigens, or one or more parasite antigens.

In some aspects, the immune response is to a bacterial antigen selected from the group consisting of a Yersinia pestis antigen or a Mycobacterium tuberculosis antigen. In some aspects, the Yersinia pestis antigen is a Yersinia pestis capsular antigen. In some aspects, the Yersinia pestis capsular antigen is F1-Ag or virulence antigen (V-Ag). In some aspects, the Mycobacterium tuberculosis antigen is an Apa antigen, an HP65 antigen, or a rAg85A antigen.

In some aspects, the immune response is to a viral antigen is selected from the group consisting of: an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a orthopoxvirus antigen, a monkeypox virus antigen, a vaccinia virus antigen, a smallpox virus antigen, a Epstein bar virus antigen, a nipha virus antigen, a varicella-zoster virus antigen. In some aspects, the enterovirus antigen is an enterovirus 71 (E71) antigen or a coxsackievirus (Cox) protein antigen. In some aspects, the E71 antigen is an E71-VP1 antigen or a glutathione S-transferase (GST)-tagged E71-VP1 antigen. In some aspects, the Cox protein antigen is GST-tagged Cox protein antigen. In some aspects, the HSV antigen is an HSV-1 envelope antigen, an HSV-2 envelope antigen, or an HSV-2 surface glycoprotein antigen. In some aspects, the HSV-2 surface glycoprotein antigen is a gB2 antigen, a gC2 antigen, a gD2 antigen, or a gE2 antigen. In some aspects, the HIV antigen is an Env antigen, a Gag antigen, a Nef antigen, or a Pol antigen. In some aspects, the HPV antigen is a minor capsid protein L2 antigen. In some aspects, the minor capsid protein L2 antigen comprises one or more epitope domains (amino acids 10-36 and/or amino acids 65-89) of minor capsid protein L2. In some aspects, the HPV antigen is a human papillomavirus type 16 Regulatory protein E2 antigen, a human papillomavirus type 16 Protein E6 antigen, a human papillomavirus type 16 Protein E7 antigen, a human papillomavirus type 18 Regulatory protein E2 antigen, a human papillomavirus type 18 Protein E6 antigen, a human papillomavirus type 18 Protein E7 antigen, a human papillomavirus type 6a Regulatory protein E2 antigen, a human papillomavirus type 6a Protein E6 antigen, a human papillomavirus type 6a Protein E7 antigen, a human papillomavirus 11 Regulatory protein E2 antigen, a human papillomavirus 11 Protein E6 antigen, a human papillomavirus 11 Protein E7 antigen. In some aspects, the HCV antigen is a nonstructural 3 (NS3) antigen, a hepatitis C virus genotype 1a Genome polyprotein antigen, a hepatitis C virus genotype 1b Genome polyprotein antigen, a hepatitis C virus genotype 2a Genome polyprotein antigen, a hepatitis C virus genotype 3a Genome polyprotein antigen. In some aspects, the RSV antigen is an F antigen or a G antigen. In some aspects, the Dengue virus antigen is an E protein antigen, an E protein domain III (EDIII) antigen, a non-structural protein 1 (NS1) antigen, or a DEN-80E antigen. In some aspects, the Ebola virus antigen is a spike glycoprotein (GB) antigen, a VP24 antigen, a VP40 antigen, a nucleoprotein (NP) antigen, a VP30 antigen, or a VP35 antigen. In some aspects the Zika virus antigen is an envelope domain III antigen or a CKD antigen. In some aspects, the Chikungunya virus antigen is an E1 glycoprotein subunit antigen, the MHC class I epitope PPFGAGRPGQFGDI (SEQ ID NO: 34), the MHC class I epitope TAECKDKNL (SEQ ID NO: 35), or the MHC class II epitope VRYKCNCGG (SEQ ID NO: 36). In some aspects, the measles virus antigen is a hemagglutinin protein MV-H antigen or a fusion protein MV-F antigen. In some aspects, the MERS-CoV antigen is a spike (S) protein antigen, an antigen from the receptor-binding domain of the S protein, or an antigen from the membrane fusion domain of the S protein. In some aspects, the SARS-CoV antigen is a spike (S) protein antigen, an antigen from the receptor binding domain of the S protein, an antigen from the membrane fusion domain of the S protein, an envelope (E) protein antigen, or an M protein antigen. In some aspects, the monkeypox antigen is a A35R protein antigen, a H3L protein antigen, or a L1R protein antigen. In some aspects, the Epstein-Barr virus antigen is an Epstein-Barr virus (strain B95-8) nuclear antigen 1 antigen, an Epstein-Barr virus (strain B95-8) Envelope glycoprotein B antigen, an Epstein-Barr virus (strain B95-8) Envelope glycoprotein H antigen, an Epstein-Barr virus (strain B95-8) Envelope glycoprotein GP350 antigen, an Epstein-Barr virus (strain B95-8) Latent membrane protein 1 antigen, or an Epstein-Barr virus (strain B95-8) Latent membrane protein 2 antigen. In some aspects, the Vaccinia virus is a Vaccinia virus (strain Western Reserve) Protein A27 antigen, a Vaccinia virus (strain Western Reserve) EEV membrane phosphoglycoprotein antigen, a Vaccinia virus B5R (Fragment) antigen, a Vaccinia virus Envelope protein H3 antigen, a Vaccinia virus (strain Western Reserve) IMV membrane protein antigen. In some aspects, the Nipah virus is a Nipah virus Fusion glycoprotein FO antigen, a Nipah virus Glycoprotein G antigen. In some aspects, the Varicella-zoster virus is a Varicella-zoster virus (strain Dumas) Envelope glycoprotein E antigen.

In some aspects, the immune response is to one or more influenza virus antigens from any influenza virus type or subtype. In some aspects, the one or more influenza virus antigens are selected from the group consisting of an influenza virus hemagglutinin (HA) antigen, an influenza virus neuraminidase (NA) antigen, an influenza virus matrix-2 (M2) protein antigen, antigenic fragments thereof, and any combination thereof. In some aspects, the one or more influenza virus antigens are derived from influenza virus type A, type B, type C, type D, or any combination thereof. In some aspects, the one or more influenza virus antigens are derived from influenza virus type A. In some aspects, the one or more influenza virus antigens derived from influenza virus type A have (a) a HA subtype selected from H1 through H18 or any combination thereof and (b) a NA subtype selected from N1 through N11 or any combination thereof. In some aspects, the one or more influenza virus antigens derived from influenza virus type A, subtype H1N1; influenza virus type A, subtype H2N2; influenza virus type A, subtype H3N2; influenza virus type A, subtype H5N1; influenza virus type A, subtype H7N7; influenza virus type A, subtype H7N9; influenza virus type A, subtype H9N2; or any combination thereof. In some aspects, the one or more influenza virus antigens are derived from influenza virus type A, subtype H1N1; influenza virus type A, subtype H3N2; or the combination thereof. In some aspects, the one or more influenza virus antigens are derived from influenza virus type B. In some aspects, the immune response is to one or more SARS-CoV-2 antigens or antigenic fragments thereof disclosed herein and one or more influenza virus antigens or antigenic fragments thereof disclosed herein. In some aspects, the immune response is to two or more SARS-CoV-2 antigens or antigenic fragments thereof disclosed herein. In some aspects, the immune response is to two or more SARS-CoV-2 antigens or antigenic fragments thereof disclosed herein that are from different strains of SARS-CoV-2. In some aspects, the immune response is to two or more different variants of the same SARS-CoV-2 protein or antigenic fragment thereof, wherein the different variants of the same SARS-CoV2 protein or antigenic fragment thereof are derived from different strains of SARS-CoV-2. In some aspects, the immune response is to two or more SARS-CoV-2 S proteins or antigenic fragments thereof from different strains of SARS-CoV-2. In some aspects, the SARS-CoV-2 S proteins or antigenic fragments thereof from different strains of SARS-CoV-2 comprise one or more mutations previously reported in Li, T. et al., Emerg Microbes Infect. 9(1):2076-90 (2020); Lee, P. et al., Immune Netw. 21(1):e4 (2021); Yu, J. et al., Science 369(6505):806-11 (2020); Cattin-Ortola, J. et al., Nat Commun. 12(1):5333 (2021); Corbett, K. et al., Nature 586(7830):567-71 (2020); Hsieh, C. et al., Science 369(6510):1501-5 (2020); and Harvey, W. et al., Nat Rev Microbiol. 19(7):409-24 (2021), each of which is incorporated by reference herein in its entirety. In some aspects, the different strains of SARS-CoV-2 are selected from the group consisting of: an Alpha SARS-CoV-2 strain (e.g., strains B.1.1.7 and Q.1-Q.8); a Beta SARS-CoV-2 strain (e.g., strains B.1.351, B.1.351.2, and B.1.351.3); a Delta SARS-CoV-2 strain (e.g., strain B.1.617.2 and AY.1 sublineages); a SARS-CoV-2 strain Gamma strain (e.g., strains P.1, P.1.1, and P.1.2); an Epsilon SARS-CoV-2 strain (e.g., strains B.1.427 and B.1.429); an Eta SARS-CoV-2 strain (e.g., strain B.1.525); an Iota SARS-CoV-2 strain (e.g., strain B.1.526); a Kappa SARS-CoV-2 strain (e.g., strain B.1.617.1); a Lambda SARS-CoV-2 strain; a B.1.617.3 SARS-CoV-2 strain; a Mu SARS-CoV-2 strain (e.g., strains B.1.621 and B.1.621.1); an Omicron strain (e.g. B.1.1.529/BA.1, BA.2, BA.5, BA.2.75, BQ.1, XBB1.5, and 22E), a Zeta strain (e.g., strain P.2); and any combination thereof. In some aspects, the different strains of SARS-CoV-2 are selected from the group consisting of: B.1.1.7, Q.1, Q.2, Q.3, Q.4, Q.5, Q.6, Q.7, Q.8, B.1.351, B.1.351.2, B.1.351.3, B.1.617.2, B.1.1.529, BA.5, BA.2.75, BQ.1, XBB1.5, 22E, AY.1 sublineages, P.1, P.1.1, P.1.2, B.1.427, B.1.429, B.1.525, B.1.526, B.1.617.1, B.1.617.3, B.1.621, B.1.621.1, P.2, and any combination thereof.

In some aspects, the immune response is to a parasite antigen, wherein the parasite antigen is a protozoan antigen. In some aspects, the immune response is to a parasite antigen selected from the group consisting of a Toxoplasma gondii antigen or a Plasmodium falciparum antigen. In some aspects, the Toxoplasma gondii antigen is antigen MIC8. In some aspects, the Plasmodium falciparum antigen is a SERA5 polypeptide antigen, or a circumsporozite protein antigen. In some aspects, the immune response is to a parasite antigen, wherein the parasite antigen is a parasitic or pathogenic fungus antigen. In some aspects, the immune response is to a parasite selected from the group consisting of a Candida spp. antigen (e.g., a Candida albicans antigen, a Candida glabrata antigen, a Candida parapsilosis antigen, a Candida tropicalis antigen, a Candida lusitaniae antigen, a Candida krusei antigen), a Pneumocystis spp. antigen, a Malassezia spp. antigen (e.g., a Malassezia furfur antigen), an Aspergillus fumigatus antigen, a Cryptococcus spp. antigen (e.g., a Cryptococcus neoformans antigen, a Cryptococcus gattii antigen), a Histoplasma capsulatum antigen, a Blastomyces dermatitidis antigen, a Paracoccidioides spp. antigen (e.g., a Paracoccidioides brasiliensis antigen, a Paracoccidioides lutzii antigen), a Coccidioides spp. antigen (e.g., a Coccidioides immitis antigen, a Coccidioides posadasii antigen), a Penicillium marneffei antigen, a Sporothrix schenckii antigen, a Trichosporon asahii antigen, a Fusarium spp. antigen (e.g., a Fusarium solanum antigen, a Fusarium oxysporum antigen), a Nectria spp. antigen, a Pseudoallescheria boydii antigen, a Cladophialphora bantianum antigen, a Ramichloridium spp. antigen, a Dactylaria gallopava antigen, an Exophiala spp. antigen (e.g., an Exophiala jeanselmei antigen, an Exophiala dermatitidis antigen), a Curvularia spp. antigen, a Bipolaris spp. antigen, an Alternaria spp. antigen, a Lacazia loboi antigen, a Conidiobolus spp. antigen (e.g., a Conidiobolus coronatus antigen, a Conidiobolus incongruus antigen), and any combination thereof.

In some aspects, the composition, pharmaceutical composition, vector, or vaccine is administered to the subject by an intramuscular, transdermal, subcutaneous, intralymphatic, intranasal, or intraperitoneal route of administration. In some aspects, the composition, pharmaceutical composition, vector, or vaccine is administered once, twice, three times, four times, five times, six times, or between seven and twenty times. In some aspects, the composition, pharmaceutical composition, vector, or vaccine is administered more than once in an interval of from 1 day to about 21 days; about 2 days to about 18 days; about 3 days to about 14 days; or about 7 days; about 10 days; about 14 days; about 21 days; about 28 days; about 35 days; about 42 days; about 49 days or about 56 days. In some aspects, the composition, pharmaceutical composition, vector, or vaccine is administered twice in an interval of about 7 days; about 10 days; about 14 days; about 21 days; about 28 days about 35 days or about 42 days. In some aspects, the composition, pharmaceutical composition, vector, or vaccine is administered from about 2 times to about 20 times with intervals of about 1 week; 2 weeks; 3 weeks; 4 weeks; 5 weeks; 6 weeks; 7 weeks; 8 weeks; 9 weeks; 10 weeks; 11 weeks; 12 weeks; 13 weeks or about 14 weeks between each administration.

### IX. Methods of Making the Compositions or Vaccines

The present disclosure also features methods of making any composition, pharmaceutical composition, or vaccine described or exemplified herein.

In some aspects, the methods comprise the steps of: (a) combining a delivery component disclosed herein with a polynucleotide disclosed herein, (b) lyophilizing the combined delivery component and polynucleotide to a powder, and (c) reconstituting the powder with a diluent to form a solution of nucleic acid complexed with the delivery component

In some aspects, the methods comprise the steps of: (a) combining a delivery component disclosed herein with a polynucleotide disclosed herein, (b) lyophilizing the combined delivery component and polynucleotide to a powder, and (c) reconstituting the powder with a diluent to form a solution of nucleic acid complexed with the delivery component, wherein the diluent comprises an adjuvant and/or a STING agonist.

In some aspects, the methods comprise combining an adjuvant with the diluent. In some aspects, the methods comprise combining a STING agonist with the diluent.

In some aspects, the methods comprise combining an adjuvant and/or a STING agonist with the reconstituted polynucleotide/delivery component solution. In some aspects, the methods comprise combining a STING agonist with the diluent and an adjuvant with reconstituted polynucleotide/delivery component solution. In some aspects, the methods comprise combining an adjuvant with the diluent and a STING agonist with the reconstituted polynucleotide/delivery component solution.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein and the amino acid or nucleotide sequences (e.g., GenBank numbers and/or Uniprot numbers), are incorporated herein by reference in their entireties.

The following examples are offered by way of illustration and not by way of limitation.

### EXAMPLES

### Example 1: Vector Construction

DNA plasmids vectors are constructed with the elements as shown in FIGS. 1-14AO. The starting plasmid backbone used vector construction contains multiple restriction sites that are utilized for subcloning the desired expression cassettes in order to generate the DNA plasmid constructs. Each expression cassette that is cloned into the vector contains all of the necessary components required for expressing the function protein or antigen (e.g promoter sequence, gene sequence, poly A tail sequence). The general methodology consists of digesting the starting DNA plasmid backbone with the appropriate restriction digest enzyme. The insert sequence is obtained from isolating and gel purifying a genetic sequence from a second DNA plasmid using appropriate restriction digest enzymes. Alternatively, the sequences to be inserted into the plasmid vector are generated synthetically or a combination of the previous two approaches can be used. The starting backbone and insert sequences are mixed together and ligated using DNA ligase. The resulting DNA plasmids are then transformed into E. coli using standard protocols and streaked onto LB agar plates containing the appropriate antibiotics. Bacterial colonies are subsequently isolated and grown in medium so that the plasmid can be purified and screened by restriction digest and gel electrophoresis in order to identify the colonies that have the correct plasmids.

### Example 2: Vector Formulation with the Delivery Component

The present disclosure can include a nucleic acid (e.g., a DNA plasmid vector or multicistronic mRNA vector) complexed with a biodegradable cross-linked cationic multi-block copolymer of the following formula:

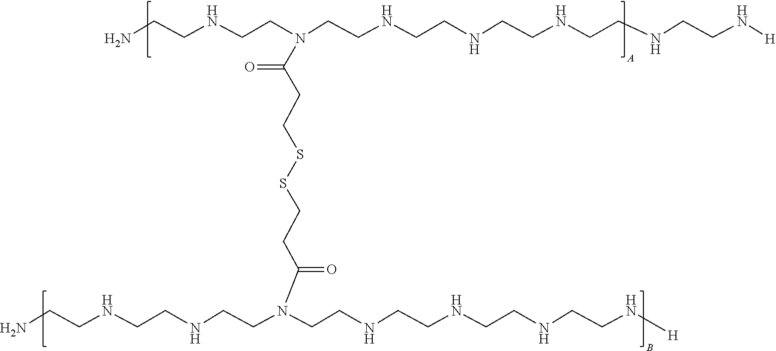

wherein: A and B are such that the molecular weight of the individual linear polyethylenimine chains are from 5,000 to 20,000 Dalton; the intermolecular crosslinks connect approximately 5-10% of amines; the biodegradable crosslinks are dithiodipropionyl (each half composed of 3 carbon atoms) and can be from 1-10 carbon atoms.

In some aspects, the biodegradable cross-linked cationic polymer comprises 10,000 to 15,000 Dalton linear PEI covalently connected with a dithiopropionyl linkage (see, e.g., U.S. Pat. No. 8,445,017). The polymer is dissolved in sterile water to give a final concentration of 3 mg/ml. The DNA is dissolved in sterile water to give a final concentration of 1 mg/ml. To make the polymer/DNA complex, the two components are diluted separately with 5% glucose to a volume of 150 uL each, and then the plasmid DNA solution is added to the polymer solution.

In some aspects, the biodegradable cross-linked cationic polymer comprises 10,000 to 15,000 Dalton linear PEI covalently connected with a dithiopropionyl linkage (see, e.g., U.S. Pat. No. 8,445,017) and is further conjugated to polyethyleneglycol (PEG) of molecular weight ranging from 500 to 20,000 Dalton. The resultant polymer is dissolved in sterile water to give a final concentration of 3 mg/ml. The DNA is dissolved in sterile water to give a final concentration of 1 mg/ml. To make the polymer/DNA complex, the two components are diluted separately with 5% glucose to a volume of 150 uL each, and then the plasmid DNA solution is added to the polymer solution.

In some aspects, the biodegradable cross-linked cationic polymer comprises 10,000 to 15,000 Dalton linear PEI covalently connected with a dithiopropionyl linkage (see, e.g., U.S. Pat. No. 8,445,017) and is further conjugated to polyethyleneglycol (PEG) of molecular weight ranging from 500 to 20,000 Dalton. The resultant polymer is dissolved in sterile water to give a final concentration of 3 mg/ml. The DNA is dissolved in sterile water to give a final concentration of 1 mg/ml. To make the polymer/DNA complex, the two components are diluted separately with 5% glucose to a volume of 150 uL each, and then the plasmid DNA solution is added to the polymer solution.

In some aspects, the biodegradable cross-linked cationic polymer comprises 15,000 to 20,000 Dalton linear PEI covalently connected with a dithiopropionyl linkage (see, e.g., U.S. Pat. No. 8,445,017). The polymer is dissolved in sterile water to give a final concentration of 3 mg/ml. The DNA is dissolved in sterile water to give a final concentration of 1 mg/ml. To make the polymer/DNA complex, the two components are diluted separately with 5% glucose to a volume of 150 uL each, and then the plasmid DNA solution is added to the polymer solution.

In some aspects, the biodegradable cross-linked cationic polymer comprises 15,000 to 20,000 Dalton linear PEI covalently connected with a dithiopropionyl linkage (see, e.g., U.S. Pat. No. 8,445,017) and is further conjugated to polyethyleneglycol (PEG) of molecular weight ranging from 500 to 20,000 Dalton. The resultant polymer is dissolved in sterile water to give a final concentration of 3 mg/ml. The DNA is dissolved in sterile water to give a final concentration of 1 mg/ml. To make the polymer/DNA complex, the two components are diluted separately with 5% glucose to a volume of 150 uL each, and then the plasmid DNA solution is added to the polymer solution.

Complex formation is allowed to proceed for 15 minutes at room temperature. To study the effect of the charge ratio on gene transfer, DNA complexes with biodegradable cross-linked cationic polymer can be prepared at different ratios 1/15/1, 10/1, and 20/1 nitrogen/phosphate (N/P). Following complex formation, the complexes are diluted in a cuvette for measurement of particle size and the potential of the complex. The electrophoretic mobility of the samples can be measured at 25° C., and at a wavelength of 657 nm and at a constant angle of 900 with a Particle sizer.

### Example 3: Vector Formulation with the Delivery Carrier PPC

Bench-scale production of highly concentrated liquid formulations of fully condensed nucleic acid with a cationic lipopolymer was prepared. This involves preparation of nucleic acid complexes with a cationic polymer followed by lyophilization and reconstitution to isotonic solutions. The nucleic acid used is a plasmid DNA, and the polymer comprises a PEI backbone covalently linked to PEG and cholesterol (PEG-PEI-cholesterol (“PPC”)). The molar ratio between PEG and PEI and between cholesterol and PEI was 0.5-10 and 0.1-10, respectively. First, the DNA and PPC solutions were separately prepared at 5 mg/ml in water for injection and subsequently diluted to 0.3 mg/ml (DNA) and 1.108 mg/ml (PPC) in 3% lactose. The DNA in lactose solution was added to the PPC in lactose solution using a micropipette to a nitrogen to phosphate ratio (N:P ratio) of 0.01:1 to 11:1, and the formulation is incubated for 15 minutes at room temperature to allow the complexes to form. The PPC/DNA complexes in 3% lactose are lyophilized using a FREEZONE freeze dry System from LABCONCO Corp. Kansas City, Mo. 500 ul of prepared formulation is added to 2 ml borosilicate glass vials which were then lyophilized using a freeze drying program consisting of the following segments:
1) freezing segment (ramp 0.25° C./min, hold at 34° C. for 4 hours),
2) primary drying segment (hold at 34° C. for 24 hrs),
3) secondary drying segment (ramp to 20° C. and hold for 24 hrs), and
4) ramp to 4° C. at 0.25° C./min.

The resultant lyophilized powder was reconstituted with 150 μl water for injection to make 0.5 mg/ml DNA.

### Example 4: Vector Formulation with the Delivery Carrier Poloxamer

Poloxamers were gently mixed with 1 mg/ml of nucleic acids in water or saline solution (0.15M) at variable concentrations. Formulated poloxamer (5%)/plasmid solutions were analyzed by gel electrophoresis in order to verify interaction between formulated plasmid and poloxamer. Comparison between unformulated plasmid DNA and DNA formulated with poloxamer had the similar movement through the gel and therefore indicated no binding between plasmid DNA and poloxamer. The formulated plasmid with poloxamer was used for gene transfer in mammalian cell or tissue.

### X.5 Synthesis of Aza-Crown-Linked Poloxamer (Crown Poloxamer)

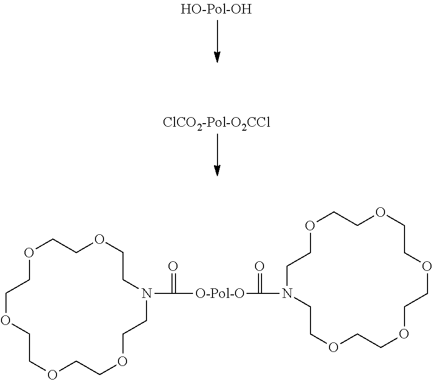

An aza-crown-linked poloxamer (crown poloxamer) was constructed as follows. Poloxamer 124 (Pluronic L-44; 500 mg, 220 mol) was dissolved in toluene (3 ml), and the resulting solution was treated with 2 ml (4 mmol) of 2M phosgene solution in toluene. After 3 hrs at room temperature, the mixture was concentrated in vacuum, the residue was re-dissolved in 3 ml toluene and concentrated again. The residue was dissolved in dry chloroform (5 ml). To this solution was added aza-18-crown-6 [1-aza-4, 7, 10, 13, 16-pentaoxacyclooctadecane (125 mg, 500 mol) and Hunig' s base (100 μl, 574 mol). After 70 hrs the reaction mixture was concentrated in vacuum, the residue was re-dissolved in distilled water and dialyzed [membrane cutoff 1000 Da] against distilled water. Concentration of the dialyzate afforded 410 mg of the title compound. Proton NMR (D₂O): 4.20 ppm (t, CH₂OC═O); 3.7-3.5 ppm [(—CH₂—CH₂—O—), both crown and poloxamer)]; 3.4 ppm (m, crown CH₂N); 1.1 ppm (m, poloxamer-(CH₃)CH—CH₂—).

### Example 5: pVac Vector Construction

DNA vector constructs for expressing viral antigens were prepared. In particular, vectors referred to as pVac 1-36 were created to express the S1 subunit of the SARS-CoV-2 S protein (SP1 or Spike1, which corresponds to amino acids 15-695 of the full-length S protein) or a SARS-CoV-2 full-length S protein (SARS2S-D614G; 1273 amino acids in length), both of which contain a D614G substitution (FIG. 13A). For constructs, pVac1-pVac5, the S protein coding sequence of each vector is under the control of a mammalian EF-1α promoter. The plasmids pVac3, pVac6, and pVac7 were designed to co-express the SARS-CoV-2 M antigen. The pVac3 and pVac6 plasmids included an Internal Ribosome Entry Site (IRES) sequence between the viral antigens. The pVac2, pVac3 and pVac5 plasmids were designed to further co-express the human heterodimeric cytokine IL12. The coding sequences for the IL-12 p35 and IL-12 p40 subunits are under the control of two CMV promoters (FIG. 13B). An unmodified pUNO Spike including the full-length SARS-CoV-2 S protein was also tested (Invivogen (San Diego, CA)).

Construction of pVac 3 (FIG. 14D) included using a TLO1001-SP1-UniRE vector and fragment MHisPA-unqRE (TWIST, San Francisco, CA; SEQ ID NO: 67). First, a p2CMV vector was engineered to express IL-12 p35 and IL-12 p40 from two CMV promoters and SARS-CoV-2 full-length D614G S protein from a hEF1-HTLV promoter (p2CMV mIL-12+SARS2S-D614G). The resulting p2CMV mIL-12+SARS2S-D614G vector was cut by AgeI/SapI (7677+4256 bp) as backbone. PCR of AgeImHisPA insert was completed using MHispA-unique RE template with primers AgeIMhis-F (SEQ ID NO: 51) and pAcelsion-R (SEQ ID NO: 52). Following PCR, Gibson assembly cloning was used to assemble an intermediate vector including p2CMV mIL-12+SARS2S-D614G and MHispA-unique RE. To complete pVac 3 construction, the intermediate vector was digested by AgeI/SalI-HF (8653+46 bp). A AgeIspIRESSalI PCR product was amplified from a TLO1001-SP1-UniRE template using the primers CelsionAgeIsp1-F (SEQ ID NO: 53) and IRESSalI-R (SEQ ID NO: 54). The AgeIspIRESSalI PCR product was then digested by AgeI/SalI-HF and ligated by T4 ligase.

Construction of pVac 2 (FIG. 14C) included digesting the pVac 3 vector (p2CMV mIL-12+Spike1-IRES-M-His-pA) by PspXI/SapI (9782+1613 bp), followed by CIP treatment and column purification. PCR for the insert was done using KOD Hot Start DNA Polymerase with the pVac3 template (p2CMV mIL-12+Spike1-IRES-M-His-pA) and PspXI-SV40 pA-F (SEQ ID NO: 56) and Cls-SapI-R (SEQ ID NO: 57) primers.

The pVac 1 vector (FIG. 14B) was constructed using a p2CMV-v4 vector as a backbone. The p2CMV-v4 vector is shown in FIG. 14I. The p2CMVv4 vector was cut with BglII/XmnI (sequential digestion: 4693+400 bp) then followed with CIP treatment and Column purification. Using a p2CMV mIL-12+Spike1-pA unique RE vector as a template, PCR was performed with Cls-BglII-R-2 (SEQ ID NO: 58) and Cls-XmnI-R (SEQ ID NO: 59) primers using KOD Hot Start DNA Polymerase. The vector was then completed using Gibson Assembly.

To construct pVac 5 (FIG. 14F), an intermediate vector with full Spike1-D614G-OP, Spike1-KanR was synthesized. Next, the intermediate vector was digested by AgeI-HF/PspXI yielding constructs of 3848, 1772, and 445 bp. Additionally, the template from pVac3 (p2CMV mIL-12+Spike1-pA unique RE) step 1 was also digested by AgeI-HF/PspXI (Cutsmart) yielding constructs of 7927 and 2105 bp. The resulting constructs were ligated by T4 DNA ligase, and clones were verified by Sanger sequencing.

To construct pVac4 (FIG. 14E), an intermediate vector with full Spike1-D614G-OP, Spike1-KanR was synthesized. Next the intermediate vector was digested by AgeI-HF/PspXI yielding constructs of 3848, 1772, and 445 bp. Additionally, the template from pVac3 p2CMV mIL-12+Spike1-IRES-M-His-pA unique RE was also digested by AgeI-HF/PspXI yielding constructs of 7927 and 2105 bp. The resulting constructs were ligated by T4 DNA ligase and clones were verified by Sanger sequencing.

To construct pVac6 (FIG. 14G) a human codon optimized M protein DNA fragment was synthesized. Vector p2CMV mIL-12+Spike1-D614G-OP-pA unique was digested by PspXI and treated by CIP. PCR for the IRES fragment from p2CMV mIL-12 +Spike1-IRES-M-His-pA was completed using CLS-PspXI-IRES-F (SEQ. ID NO. 60) and IREScelsion-R (SEQ. ID NO. 61) primers. PCR of the M protein from synthesized fragment was done using Mhiscelsion-F (SEQ. ID NO. 62) and CLS-M-R (SEQ. ID NO. 63) primers. Gibson Assembly was used with the two inserts to create vector p2CMV mIL-12+Spike1-D614G-HuOP-IRES-M-HuOP-pA mock.

To construct pVac7 (FIG. 14H), p2CMV-V4-Spike1-D614G-OP-pA unique RE (the pVac3 intermediate vector) was digested by MluI-HF and treated by CIP. PCR of the M protein from synthesized fragment was completed using CLS-XbaI-M-F (SEQ. ID NO. 64) and CLS-MluI-M-R (SEQ. ID NO. 65) primers. Gibson Assembly was used with the two inserts to create vector p2CMV M-HuOP+Spike1-D614G-HuOP-pA mock.Construct pVac8 (FIG. 14J) was generated to express the SARS-CoV-2 Spike Protein with D614G mutation and deletion of the last 17 amino acids of the C terminal domain (pVac4-C-del-mock). Construct pVac9 (FIG. 14K) was generated to express the SARS-CoV-2 Spike Protein with D614G mutation and 2P modification at amino acids 986(K) and 987(V) (pVac4-KV-PP). Construct pVac10 (FIG. 14L) was generated to express the full length SARS-CoV-2 Spike Protein (CLS-V4-CMV-S-D614G-mock-step1). Construct pVac 11 (FIG. 14M) was generated to express the full length SARS-CoV-2 delta variant Spike Protein under the control of a CMV promoter (CLS-pCMV-Delta-S-OP-mock). Construct pVac12 (FIG. 14N) was generated to express the full length SARS-CoV-2 Spike Protein under the control of a CMV promoter and the SARS-CoV-2 full length delta variant Spike Protein under the control of a CMV promoter (CLS-p2CMV-S-D614G-Delta-S-OP-mock). Construct pVac13 (FIG. 14O) was generated to express the full length SARS-CoV-2 Spike Protein with the aid of an SV40 Enhancer sequence (CLS-pVac4modify-SV40E-EF 1a-mock). Construct pVac14 (FIG. 14P) was generated to express the SARS-CoV-2 Spike Protein with the C terminal deletion and 2P modification (CL S-pVac4modify-KVPP-C-del-mock). Construct pVac15 (FIG. 14Q) was generated to express the full length SARS-CoV-2 Spike Protein with the 2P modification driven by a CMV promoter (CLS-V4-CMV-S-D614G-KVPP-mock). Construct pVac16 (FIG. 14R) was generated to express the full length SARS-CoV-2 delta variant Spike Protein with the 2P modification driven by a CMV promoter (CLS-p2CMV-Delta-S-OP-KVPP-mock). Construct pVac17 (FIG. 14S) was generated to express the full length SARS-CoV-2 Spike Protein with the 2P modification and expressing the SARS-CoV-2 full length delta variant Spike Protein with the 2P modification under the control of a CMV promoter (CLS-p2CMV-S-D614G-KVPP-Delta-S-OP-KVPP-mock). Construct pVac18 (FIG. 14T) was generated to express the SARS-CoV-2 Spike Protein with the C terminal deletion and 2P modification and expressing the SARS-CoV-2 delta variant Spike Protein with the C terminal deletion and 2P modification under the control of an EF1-alpha promoter (CLS-EF-D614G-PP-Cdel-CMV1-Delta-PP-Cdel-mock). Construct pVac19 (FIG. 14U) was generated to express the SARS-CoV-2 Spike Protein with the C terminal deletion and 2P modification and express the SARS-CoV-2 delta variant Spike Protein with the C terminal deletion and 2P modification under the control of a CMV promoter (CLS-p2CMV-S-D614G-PP-Cdel-Delta-S-OP-PP-Cdel-mock). Construct pVac20 (FIG. 14V) was generated to express the SARS-CoV-2 Spike Protein with the C terminal deletion and 2P modification driven by a CMV promoter (CLS-V4-CMV2-S-D614G-PP-Cdel-mock). Construct pVac21 (FIG. 14W) was generated to express the SARS-CoV-2 delta variant Spike Protein with the C terminal deletion and 2P modification driven by a CMV promoter (CLS-V4-CMV1-Delta-PP-Cdel-mock). Construct pVac22 (FIG. 14X) was generated to express the SARS-CoV-2 Spike Protein with the C terminal deletion driven by a CMV promoter (CLS-V4-CMV2-S-D614G-Cdel-mock). Construct pVac23 (FIG. 14Y) was generated to express the SARS-CoV-2 delta variant Spike Protein with the C terminal deletion driven by a CMV promoter (CLS-V4-CMV1-Delta-Cdel-mock). Construct pVac24 (FIG. 14Z) was generated to express the SARS-CoV-2 Spike Protein with the C terminal deletion driven by a CMV promoter and expresses the SARS-CoV-2 delta variant Spike Protein with the C terminal deletion driven by a CMV promoter (CLS-p2CMV-S-D614G-Cdel-Delta-S-OP-Cdel-mock). Construct pVac25 (FIG. 14AA) was generated to express the SARS-CoV-2 Spike Protein with the C terminal deletion and 2P modification driven by a CMV promoter and expresses the SARS-CoV-2 delta variant Spike Protein with the C terminal deletion and 2P modification driven by a CMV promoter (CLS-EF1-S-D614G-PP-Cdel-EF1-Delta-PP-Cdel-mock). Construct pVac26 (FIG. 14AB) was generated to express the SARS-CoV-2 delta variant Spike Protein with the C terminal deletion and 2P modification driven by an EF 1 promoter (CLS-V4-EF1-Delta-PP-Cdel-mock). Construct pVac27 (FIG. 14AC) was generated to express the SARS-CoV-2 delta variant Spike Protein with the 2P modification driven by an EF1 promoter (CLS-V4-EF1-Delta-PP-mock). Construct pVac28 (FIG. 14AD) was generated to express the SARS-CoV-2 Spike Protein with the 2P modification driven by an EF 1 promoter and expresses the SARS-CoV-2 delta variant Spike Protein with the 2P modification driven by an EF1 promoter (CLS-EF1-S-D614G-PP-EF1-Delta-PP-mock). Construct pVac29 (FIG. 14 AE) was generated to express the SARS-CoV-2 delta variant Spike Protein with the C terminal deletion driven by an EF 1 promoter (CLS-V4-EF1-Delta-Cdel-mock). Construct pVac30 (FIG. 14AF) was generated to express the SARS-CoV-2 Spike Protein with the C terminal deletion driven by an EF 1 promoter and expresses the SARS-CoV-2 delta variant Spike Protein with the C terminal deletion driven by an EF1 promoter (CLS-EF1-S-D614G-Cdel-EF1-Delta-Cdel-mock). Construct pVac31 (FIG. 14AG) was generated to express the SARS-CoV-2 Spike Protein with the 2P modification driven by a CMV promoter with the aid of an SV40 enhancer sequence (CLS-SV40E-CMV-S-D614G-KVPP-mock). Construct pVac32 (FIG. 14AH) was generated to express the SARS-CoV-2 delta variant Spike Protein with the 2P modification driven by a CMV promoter with the aid of an SV40 enhancer sequence (CLS-SV40E-CMV-Delta-S-OP-KVPP-mock). Construct pVac33 (FIG. 14AI) was generated to express the SARS-CoV-2 Spike Protein with the 2P modification driven by a CMV promoter with the aid of an SV40 enhancer sequence and express the SARS-CoV-2 delta variant Spike Protein with the 2P modification driven by a CMV promoter with the aid of an SV40 enhancer sequence (CLS-SV40E-CMV-S-D614G-KVPP-SV40E-CMV-Delta-S-KVPP-mock). Construct pVac34 (FIG. 14AJ) was generated to express the SARS-CoV-2 Omicron variant Spike Protein driven by a CMV promoter with the aid of an SV40 enhancer sequence (CLS-SV40E-CMV-Omi-S-mock). Construct pVac35 (FIG. 14AK) was generated to express the SARS-CoV-2 Omicron variant Spike Protein with the 2P modification driven by a CMV promoter with the aid of an SV40 enhancer sequence (CLS-SV40E-CMV-Omi-S-KVPP-mock). Construct pVac36 (FIG. 14AL) was generated to express the SARS-CoV-2 Spike Protein with the 2P modification driven by a CMV promoter with the aid of an SV40 enhancer sequence and express the SARS-CoV-2 Omicron variant Spike Protein with the 2P modification driven by a CMV promoter with the aid of an SV40 enhancer sequence (CLS-SV40E-CMV-Omi-S-KVPP-SV40E-CMV-Delta-S-KVPP-mock). Construct pVac37 (FIG. 14AM) was generated to express the RSV F Protein driven by a CMV promoter with the aid of an SV40 enhancer sequence (CLS-SV40E-CMV-RSV-F-Mock). Construct pVac38 (FIG. 14AN) was generated to express the CMV Gb Protein driven by a CMV promoter with the aid of an SV40 enhancer sequence (SV40E-CMV-CMV-Gb). Construct pH1N1 Brisbane (FIG. 14AO) was generated to express the H1N1 2007 Brisbane variant HA Protein driven by a CMV promoter (H1N1 brisbane 2007 vector). Construct pVac40 (FIG. 14AP) was generated to express the SARS-CoV-2 Spike Protein with D614G mutation and the 2P modification driven by a CMV promoter with the aid of a SV40 enhancer sequence and express the luciferase protein driven by a CMV promoter with the aid of a SV40 enhancer sequence (pVac40). Construct pVac42 (FIG. 14AQ) was generated to express the SARS-CoV-2 Spike Protein with D614G mutation and the 2P modification driven by a CMV promoter (pVac42). Construct pVac43 (FIG. 14AR) was generated to express SARS-CoV-2 Spike Protein with D614G mutation and the 2P modification driven by a CMV promoter and express the full length SARS-CoV-2 delta variant Spike Protein with the 2P modification driven by a CMV promoter (pVac43). Construct pVac44 (FIG. 14AS) was generated to express SARS-CoV-2 Spike Protein with D614G mutation and the 2P modification driven by a CMV promoter with the CMV intron A (pVac44). Construct pVac45 (FIG. AT) was generated to express SARS-CoV-2 Spike Protein with D614G mutation and the 2P modification driven by a CMV promoter with the aid of a SV40 enhancer sequence and expressing the full length SARS-CoV-2 delta variant Spike Protein with the 2P modification driven by a CMV promoter with the aid of a SV40 enhancer sequence (pVac45). Construct pVac46

(FIG. 14AU) was generated to express the SARS-CoV-2 Spike Protein with D614G mutation and the 2P modification driven by a CMV promoter with a CMV intron A and the aid of a SV40 enhancer sequence and express the full length SARS-CoV-2 delta variant Spike Protein with the 2P modification driven by a CMV promoter with a CMV intron A and the aid of a SV40 enhancer sequence. Construct pVac47 (FIG. 14V) was generated to express the SARS-CoV-2 Spike Protein with D614G mutation and the 2P modification driven by a CMV promoter with a CMV intron A and the aid of a SV40 enhancer sequence (pVac47). Construct pVac48 (FIG. 14W) was generated to express the full length SARS-CoV-2 delta variant Spike Protein with the 2P modification driven by a CMV promoter with a CMV intron A and the aid of a SV40 enhancer sequence (pVac48). Construct pVac49 (FIG. 14AX) was generated to express SARS-CoV-2 Spike Protein with D614G mutation and the 2P modification driven by a CMV promoter with a CMV intron A and the aid of a SV40 enhancer sequence and express the full length SARS-CoV-2 delta variant Spike Protein with the 2P modification driven by a CMV promoter with a CMV intron A and a SV40 enhancer sequence (pVac59). Construct pVac50 (FIG. 14AY) was generated to express the full length SARS-CoV-2 delta variant Spike Protein with the 2P modification driven by a CMV promoter with the aid of an SV40 enhancer sequence (pVac50). Construct pVac51 (FIG. 14AZ) was generated to express the SARS-CoV-2 Omicron BA.2.75.2 variant Spike Protein driven by a CMV promoter (pVac51). Construct pVac52 (FIG. 14AAA) was generated to express the SARS-CoV-2 BA.2.75.2 variant Spike Protein with a leader sequence and driven by a CMV promoter (pVac52). Construct pVac53 (FIG. 14AAB) was generated to express the full length SARS-CoV-2 delta variant Spike Protein with the 2P modification driven by a CMV promoter with the aid of an SV40 enhancer sequence (pVac53). Construct pVac54 (FIG. 14AAC) was generated to express SARS-CoV-2 Spike Protein with D614G mutation and the 2P modification driven by a CMV promoter with the aid of a SV40 enhancer sequence and express the full length SARS-CoV-2 delta variant Spike Protein with the 2P modification driven by the CMV promoter with the aid of a SV40 enhancer sequence (pVac54). Construct pVac55 (FIG. 14AAD) was generated to express the SARS-CoV-2 Omicron 22E (BQ.1) variant Spike Protein with the 2P modification driven by a CMV promoter with the aid of a SV40 enhancer sequence (pVac55). Construct pVac56 (FIG. 14AAE) was generated to express the SARS-CoV-2 Omicron 22E (BQ.1) variant Spike Protein with the 2P modification and furin cleavage mutation driven by the CAG promoter with the aid of a SV40 enhancer sequence (pVac56). Construct pVac57 (FIG. 14AAF) was generated to express the SARS-CoV-2 Beta variant Spike Protein with the 2P modification and furin cleavage mutation driven by a CMV promoter with the aid of a SV40 enhancer sequence (pVac57). Construct pVac58 (FIG. 14AAG) was generated to express SARS-CoV-2 Nucleocapsid Protein driven by a CMV promoter with the aid of a SV40 enhancer sequence (pVac58). Construct pVac59 (FIG. AAH) was generated to express the SARS-CoV-2 Omicron 22E (BQ.1) variant Spike Protein with the 2P modification and furin cleavage mutation driven by the CMV promoter with the aid of the SV40 enhancer sequence and express the SARS-CoV-2 Beta variant Spike Protein with the 2P modification and furin cleavage mutation driven by the CMV promoter with the aid of the SV40 enhancer sequence (pVac59). Construct pVac60 (FIG. 14AAI) was generated to express the SARS-CoV-2 Omicron 22E (BQ.1) variant Spike Protein with the 2P modification and furin cleavage mutation driven by the CAG promoter with the aid of a SV40 enhancer sequence and express the SARS-CoV-2 Beta variant Spike Protein with the 2P modification and furin cleavage mutation driven by a CMV promoter with the aid of a SV40 enhancer sequence (pVac60). Construct pVac61 (FIG. 14AAJ) was generated to express the SARS-CoV-2 Omicron BA.5 variant Spike Protein driven by a CMV promoter with the aid of a SV40 enhancer sequence, express the SARS-CoV-2 Nucleocapsid Protein driven by a CMV promoter with the aid of a SV40 enhancer sequence, and express SARS-CoV-2 Spike Protein with D614G mutation and the 2P modification driven by a CMV promoter with the aid of a SV40 enhancer sequence (pVac61). Construct pVac62 (FIG. 14AAK) was generated to express the SARS-CoV-2 Omicron BQ.1 variant Nucleocapsid Protein driven by a CMV promoter with the aid of a SV40 enhancer sequence and express the SARS-CoV-2 Omicron XBB1.5 Spike Protein driven by a CMV promoter with the aid of a SV40 enhancer sequence (pVac62).

### Example 6: In Vivo Tests

### I. Animal Immunization Procedure

The immunogenicity study for vaccine plasmid was carried out in BALB/C mice. Female BALB/c mice (-5-6 weeks old) were used in the study (Envigo, Indianapolis). BALB/c mice immunized with various doses of vaccine (10 μg-500 g) with gaps between vaccinations of 2 weeks, 3 weeks, or 4 weeks in the absence of a booster or with one or two booster immunizations.

In one study, on Day 0, 125 μg or 250 μg of formulated DNA plasmid with aluminum phosphate and 0.5% crown poloxamer was injected intramuscularly (Quadriceps). Two weeks after first immunization, animals were given first booster dose. Blood was collected from animals on day 14 (2wp1) and 35 (3wp2). Further, IFN responses from spleenocytes were assessed on day 35.

For delivering naked DNA plasmid without crown poloxamer formulation, a 250 μg dose in phosphate-buffered saline was used for IM injections (FIG. 18).

### II. IgG Antigen S-Specific ELISA

Individual serum samples were assayed for the presence of S-specific IgG by enzyme-linked immunosorbent assay (ELISA). ELISA plates (96-well, Nunc) were coated overnight at 4° C. with 1 μg/ml purified S1 antigen in PBS. After washing (PBS with 0.05% Tween-20), plates were blocked with ChonBlock ELISA Buffer (Chondrex, Inc) for 2 h at room temperature. After blocking, the plates were washed and serial dilutions of serum in ChonBlock ELISA Buffer were performed in a separate plate. The serially diluted sera were added to the assay plate and incubated for 2 h at room temperature. After washing, plates were incubated with anti-Mouse IgG, peroxidase-linked species-specific whole antibody (from sheep) Secondary Antibody (Cytiva) diluted 1:500 in PBS for 1 h at room temperature. Finally, plates were washed and TMB peroxidase substrate solution (ThermoFisher) was added to the wells. Reactions were stopped by addition of 1M H₃PO₄ (Fisher Scientific) and absorbance was read at 450 nm on the EL808 ULTRA Microplate Reader luminometer (Winooski, VT) with Gen 5 ELISA software using a 1 second integration per well. For each serum sample, a plot of optical density (OD) versus logarithm of the reciprocal serum dilution was generated by nonlinear regression (GraphPad Prism). Titers were defined as the reciprocal serum dilution at an OD of approximately 0.5 (normalized to a standard mouse anti-S monoclonal antibody that is included on every plate).

### III. Mouse T-cell Functional Assay

Mouse T cell functional assay was done using IFN_{γ} ELISPOT assay. Spleens from immunized mice were collected in sterile tubes containing CTL-test media (Immunospot Cat #CTLT-005) media supplemented with 1× Glutamine and Penstrep. Cell suspensions were prepared by dissociating spleens in gentle Macs Octo Dissociator (Miltenyi Biotec, Auburn CA). The cell suspension was then filtered through a 70 μm cell strainer. The cells were pelleted by centrifugation at 4 C for 10 min at 300 g in a centrifuge. The pellet was resuspended in CTL media supplemented with 1× Glutamine and Penstrep. Cells were counted using the CTL cell counting program. The cell solution was diluted to contain 250,000 cells/100 μl for each well.

The 96 well mouse IFN ELISPOT kit (Mabtech USA) pre-coated plates were used for this assay. Before use, the plates were hydrated with sterile PBS 4 times (200 μl/well). The plate was then conditioned with CTL media (same as used for splenocyte isolation for at least 30 min at RT. 250,000 mouse spleenocytes were plated into each well and stimulated for 20 h with pools of 15mer peptides covering the N terminal S1 domain of the surface glycoprotein. The peptivator SARS-CoV-2 Prot contains the aa sequence 1-692 of the surface glycoprotein (Miltenyi Biotec, Cat #130-127-041). The spots were developed based on manufacturer instructions. CTL media and PMA (Invitrogen, Waltham, MA) were used for negative and positive control respectively. Spots were scanned and quantified by Immunospot CTL reader S6 (CTL, USA). Spot forming unit per 250,000 cells was calculated and averaged from duplicate wells.

### IV. SARS-CoV-2 Pseudovirus Neutralization Assay

HEK293T cells stably expressing hACE2 and TMPRSS2 (Invivogen, San Diego) were seeded on a 96 well plate for 16 hours. Mouse sera from vaccinated and naïve groups were serially diluted threefold in cell media starting at a 1:10 dilution in a separate dilution plate. Diluted sera were incubated at 37° C. for 1 hour with a fixed concentration of SARS-CoV-2 S eGFP reporter (D614G) pseudotyped lentivirus obtained from BPS Bioscience (San Diego, CA). The mouse sera and SARS-CoV-2 S pseudotyped virus mixture was then transferred to the 96 well plate containing cells and allowed to incubate in a standard incubator (37% humidity, 5% CO2) for 48 hours. GFP positive cells were imaged post-infection with the Immunospot S6 Analyzer (Cellular Technologies Limited), and brightfield images were used to confirm the presence of cells in each of the wells. The SARS-CoV-2 S pseudovirus was unable to infect normal 293T cells without the hACE2 and TRMPSS2 proteins. Neutralization titers were calculated as the serum dilution at which the number of GFP positive cells were reduced by 50% compared with virus control wells.

A high level toxicity study demonstrated that all mice receiving any of the immunizations or PBS maintained a stable weight (FIG. 19).

### Example 7: Vaccine Formulations

### I. PPC/pVaccine Formulation

Vaccine plasmid DNA was prepared initially at 0.1 mg/mL with PEG-PEI-cholesterol (PPC; MW 4.2 kD), by mixing plasmid DNA with the polymer at (11:1) and (0.5:1) (N:P) ratios in 5% dextrose solution, and the mixture was incubated at room temperature for 10 minutes to allow the formation of nanocomplexes. Then, pDNA/PPC nanocomplexes were concentrated to 1-5 mg/mL using Amicon Ultra Centrifugal Filters (Ultracel-3K MWCO).

The electrophoretic mobility of the PPC/DNA complexes was determined by agarose gel electrophoresis at 70 V for one hour. DNA integrity in the complexes was determined by incubation of the complexes with 50 μg of dextran sulfate or triton-X for 10 minutes at room temperature followed by gel electrophoresis at 100 Volts for one hour. The particle size the nanocomplexes in Milli-Q water was determined at 657 nm at a constant angle of 90° by dynamic light scattering using a Malvern particle size analyzer. Osmolality of the formulation was determined using Fiske210 micro-sample osmometer, DNA quantification was performed using spectrophotometry, formulation pH was measured using Accumet research AR15 pH meter.

### II. BD15-12/pVaccine Formulation

Vaccine plasmid DNA was prepared initially at 0.1 mg/mL with PEI-base copolymer, BD15-12 (15 kD linear PEI; MW 26.5 kD), by mixing plasmid DNA with the polymer at 10:1 and 0.5:1 N:P ratios in 5% dextrose solution, and the mixture was incubated at room temperature for 10 minutes to allow the formation of nanocomplexes. Then, BD15-12/pDNA nanocomplexes were concentrated to 2.5-5 mg/mL using Amicon Ultra Centrifugal Filters (Ultracel-3K MWCO)

The electrophoretic mobility of the BD15-12/DNA complexes was determined by agarose gel electrophoresis at 70 V for one hour. The particle size of the nanocomplexes in Milli-Q water was determined at 657 nm at a constant angle of 900 by dynamic light scattering using a Malvern particle size analyzer. The complexation of plasmid DNA with BD15-12 resulted in DNA condensation into nanoparticles and prevented DNA migration on agarose gel (data not shown). Osmolality of the formulation was determined using Fiske 210 micro-sample osmometer, DNA quantification was performed using spectrophotometry, and formulation pH was measured using Accumet research AR15 pH meter (Table 1).

TABLE 1
DNA concentration, pH, osmolality, and particle
size results of formulated vaccine plasmids

| | DNA | | | | | | |
|---|---|---|---|---|---|---|---|
| | Conc. | | | | | | |
| | mg/mL | mOsm¹ | Zave² | PdI³ | di(10)⁴ | di(50) ⁵ | di(90)⁶ |
| BD15-12/pUNO (10:1)(N:P) | 2.25 | 447 | 145.5 | 0.27 | 81.4 | 158 | 396 |
| 2.5 mg/mL | | | | | | | |
| Star:star-mPEG11(10:1)/ | 3.87 | 464 | 135.5 | 0.252 | 85.3 | 155 | 289 |
| pUNO(5:1)(N:P) 2.5 mg/mL | | | | | | | |
| Omnifect/pUNO(10:1)(N:P) | 2.09 | 451 | 84.21 | 0.295 | 42.4 | 91.2 | 291 |
| 2.5 mg/mL | | | | | | | |
| 0.5% Crown-Poloxamer/ | 2.90 | 350 | N/A | | | | |
| pUNO(5:1)(N:P) 2.5 mg/mL | | | | | | | |

¹Milliosmolality (mOSM);
²ZAverage (Zave): mean of measured particles using light scattering;
³Polydispersity index (PdI);
⁴di(10): size (nm) below which 10% of particles are found;
⁵ di(50): size (nm) below which 50% of particles are found;
⁶di(90): size (nm) below which 90% of particles are found.

### III. Staramine:Star-mPEG/pVaccine Formulation:

Staramine: Poly(ethylene glycol) methyl ether (mPEG)/pVaccine formulation is composed of Staramine (0.635 kD) liposomes and plasmid DNA. Staramine alone or 10:1 mixtures of Staramine (Star) and Star-PEG515 (1.2 kD) were rotary-evaporated to a film. The flask of liposome film was held under high vacuum overnight. Water for injection was added to the film to give the desired Staramine concentration and bath sonicated for −30 minutes using Branson Water Bath Sonicator model 2510 followed by a probe sonication for 5 minutes (using a continuous pulse sonication with an output wattage of 5-10 watts (rms) (Model 100; Fisher Scientific Sonic Desmembrator, Pittsburgh, PA). The liposome solution was filtered through a 0.2-μm filter, diluted with 5% dextrose, and mixed with the desired amount of plasmid. Particle size of the complexes was measured with Malvern particle size analyzer sizer. The complexation efficiency was determined by gel retardation assay (Life Technologies, Carlsbad, CA). The gel retardation assay was performed by loading the Staramine/plasmid DNA on 1% agarose gel and electrophoresed at 100 V for 1 hour. To release the plasmid DNA from the Staramine nanoparticles, 10% TritonX-100 was added to the complex and the solution was loaded on the agarose gel. The complexation of plasmid DNA with Staramine:Staramine-PEG resulted in DNA condensation into nanoparticles and prevented DNA migration on agarose gel. (Data not shown). The formulation procedure did not result in aggregation of DNA and staramine complexes. The addition of dextran sulfate and triton decomplexed the DNA from staramine causing a free mobility of DNA on agarose gel. The intact appearance of the DNA bands on the agarose gel for the decomplexed DNA formulation suggests that the DNA has not been degraded during formulation process. Osmolality of the formulation was determined using Fiske210 micro-sample osmometer, DNA quantification was performed using spectrophotometry, and formulation pH was measured using Accumet research AR15 pH meter (Table 1).

### IV. Omnifect/pVaccine Formulation

Vaccine plasmid DNA was prepared initially at 0.1 mg/mL with Omnifect (MW 7.3 kD), by mixing plasmid DNA with the polymer at 10:1 and 0.5:1 N:P ratios in 5% dextrose solution, and the mixture was incubated at room temperature for 10 minutes to allow the formation of nanocomplexes. Then, Omnifect/pDNA nanocomplexes were concentrated to 2.5-5 mg/mL using Amicon Ultra Centrifugal Filters (Ultracel-3K MWCO)

The electrophoretic mobility of the Omnifect/DNA complexes was determined by agarose gel electrophoresis at 70 V for one hour. The particle size the nanocomplexes in Milli-Q water was determined at 657 nm at a constant angle of 90° by dynamic light scattering using a Malvern particle size analyzer. DNA integrity in the complexes was determined by incubation of the complexes with 50 μg of dextran sulfate or triton-X for 10 minutes at room temperature followed by gel electrophoresis at 100 Volts for one hour. The complexation of plasmid DNA with Omnifect resulted in DNA condensation into nanoparticles and prevented DNA migration on agarose gel. The formulation procedure did not result in aggregation of DNA and staramine complexes. Further, the addition of dextran sulfate and triton decomplexed the DNA from Omnifect causing a free mobility of DNA on agarose gel. The intact appearance of the DNA bands on the agarose gel for the decomplexed DNA formulation suggests that the DNA had not been degraded during formulation process. (Data not shown). Osmolality of the formulation was determined using Fiske210 micro-sample osmometer, DNA quantification was performed using spectrophotometry, and formulation pH was measured using Accumet research AR15 pH meter (Table 1).

V. Crown poloxamer/pVaccine Formulation

The required concentration of plasmid DNA to produce a final concentration of 1-5 mg/ml was made in PBS or NaCl, the mixture was mixed by low speed vortexing and the required amount of crown poloxamer (MW 2.2 Kd) was add to the DNA solution to produce a final concentration of 0.1-5%.

To store the formulation at −20° C. or for lyophilization purposes, the formulation was made in 20 mM Tris-8% sucrose instead of PBS or NaCl. Osmolality of the formulation was determined using Fiske210 micro-sample osmometer, DNA quantification was performed using spectrophotometry, and formulation pH was measured using Accumet research AR15 pH meter (Table 1).

VI. Crown poloxamer/Cationic Carrier Adjuvant/pVaccine Formulation

The required concentration of plasmid DNA to produce a final concentration of 0.1-0.5 mg/ml was made in 5% dextrose or PBS, the mixture was mixed by low speed vortexing and the required amount of cationic adjuvant carrier (Staramine, BD15-12, PPC, Omnifect, Benzalkonium chloride (BAK)) was add to plasmid DNA. pDNA/PPC nanocomplexes were concentrated to 1-5 mg/mL using Amicon Ultra Centrifugal Filters (Ultracel-3K MWCO). Then crown poloxamer (i.e., aza-crown-linked poloxamer) was add to the DNA solution to produce a final concentration of 0.1-5%. Particle size of the CP-cationic carrier-pDNA complexes was measured with Malvern particle size analyzer sizer. Mixing crown poloxamer with plasmid DNA did not form any detectable nanoparticles by particle size measurements or gel electrophoresis. However, when cationic delivery systems were added to crown poloxamer/DNA formulation measurable nanoparticles were observed in the formulation. The complexation efficiency was determined by gel retardation assay (Life Technologies, Carlsbad, CA). The gel retardation assay was performed by loading the formulated plasmid DNA on 1% agarose gel and electrophoresed at 100 V for 1 hour. The intact appearance of the DNA bands on the agarose gel for crown poloxamer/DNA formulation suggested that crown poloxamer did not bind or degrade DNA during formulation process. However, a shift in DNA bands was observed when cationic delivery systems added to crown poloxamer/DNA formulation, suggesting an interaction between DNA and cationic delivery systems.

Osmolality of the formulation was determined using Fiske210 micro-sample osmometer, DNA quantification was performed using spectrophotometry, and formulation pH was measured using Accumet research AR15 pH meter (Table 2).

TABLE 2
DNA concentration, pH, osmolality, and particle
size results of formulated vaccine plasmids

| | | | | | | | | Signs of |
|---|---|---|---|---|---|---|---|---|
| | pH | mOsm¹ | Zave² | PDI³ | di(10)⁴ | di(50)⁵ | di(90)⁶ | Toxicity |
| Star:starPEG/CP/pSEAP | 4.42 | 539 | 109 | 0.377 | 45.1 | 140 | 270 | None |
| PPC/CP/pSEAP | 4.57 | 550 | 137.6 | 0.725 | 35.3 | 312 | 619 | None |
| BAK/CP/pSEAP | 6.94 | 371 | 155.1 | 0.441 | 59.5 | 204 | 373 | None |
| Omnifect/CP/pSEAP | 4.56 | 490 | 178.4 | 1 | 103 | 311 | 1230 | None |
| BD15-12/CP/pSEAP | 4.63 | 509 | 184.9 | 1 | 95 | 562 | 1560 | None |
| CP/pSEAP | 7.05 | 362 | ND⁷ | ND | ND | ND | ND | ND |

¹Milliosmolality (mOSM);
²ZAverage (Zave): mean of measured particles using light scattering;
³Polydispersity index (PdI);
⁴di(10): size (nm) below which 10% of particles are found;
⁵di(50): size (nm) below which 50% of particles are found;
⁶di(90): size (nm) below which 90% of particles are found;
⁷Not Determined (ND)

### VII. Lyophilization Cycle

1 ml of each formulation was aliquoted into 2 ml glass vials and placed into freeze-dryer (FREEZONE Triad freeze dry System from LABCONCO Corp. Kansas City, MO.). Vials were cooled to −45° C. for 16 hours, and then temperature was raised to −15° C. before the start of the primary drying. After 24 hrs, the shelf temperature was raised to 0° C. and kept under vacuum for another 24 hours. Finally, shelf temperature was raised to 25° C. for 72 hours and vials were capped under vacuum at the end of secondary drying segment.

### Example 8: In Vivo Testing of Compositions Comprising Adjuvant and/or STING Agonist

I. Single Antigen or Multiple Antigen Plasmid Vaccine Formulations Based on Crown Poloxamer Delivery Agent and an Adjuvant

Plasmid vectors including pVac-15 encoding the CMV-Spike-2P antigen D614G variant of SARS-CoV-2 (single antigen); pVac-16 encoding the CMV-Spike-2P antigen of Delta variant of SARS-CoV-2 (single antigen); and pVac-17 encoding the CMV-Spike-2P antigen D614G+Delta variant of SARS-CoV-2 (dual antigen) were administered at 125 μg plasmid DNA with 0.5% crown poloxamer and aluminum P04 adjuvant by intramuscular injection (i.m.) at day 0 (prime) and day 14 (boost) to BalbC mice. SARS-CoV-2 D614-2P mRNA and PBS were administered as controls. IgG titers were determined on day 35 after prime and showed efficient induction of IgG with pVac-15, -16, and -17 as well as the positive mRNA control (FIG. 16A).

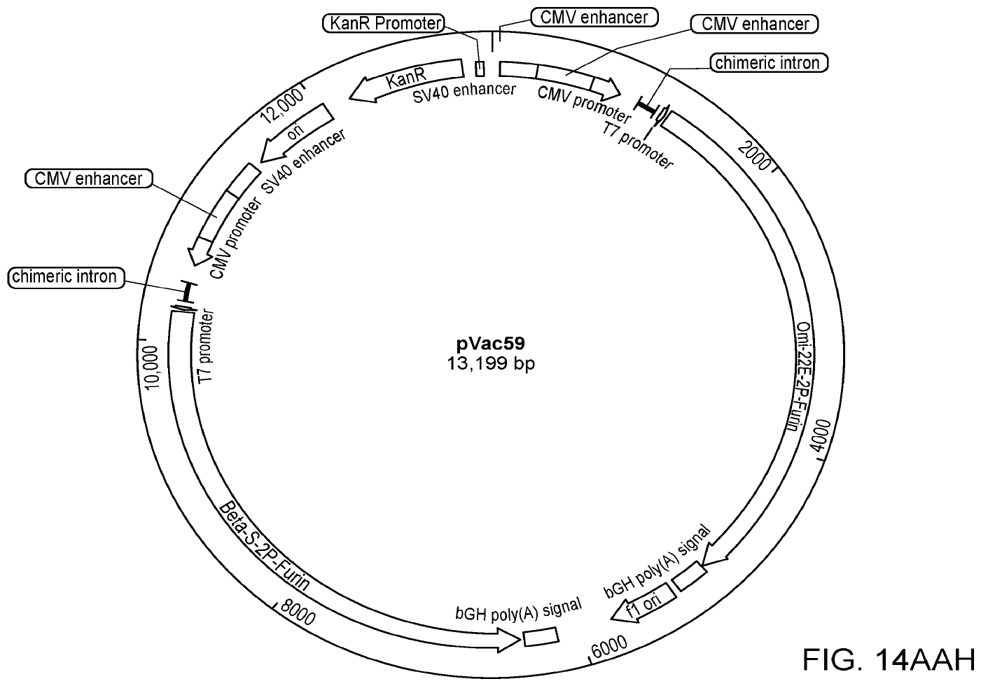

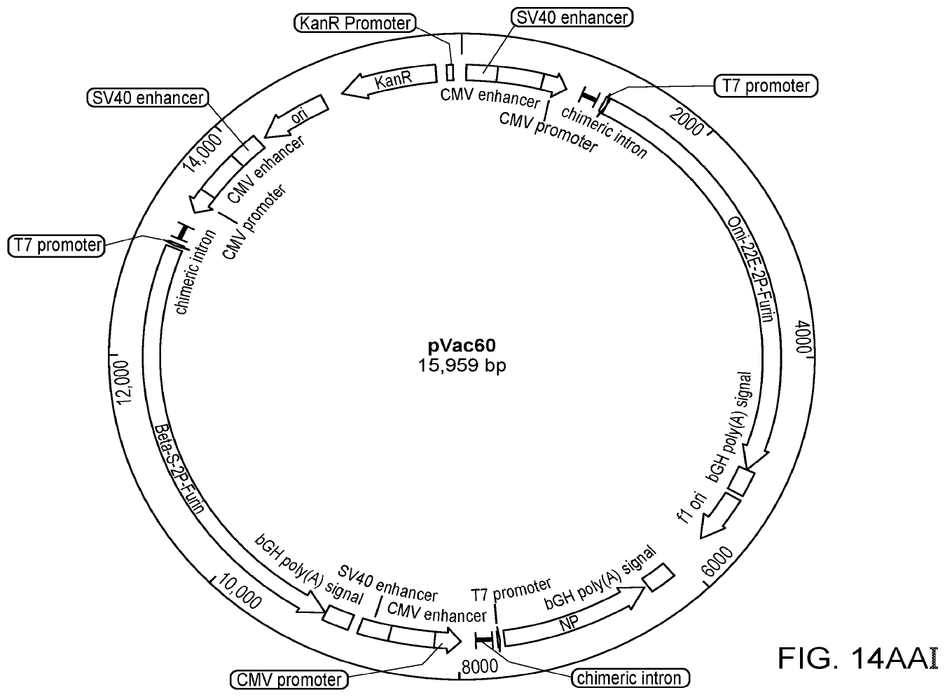

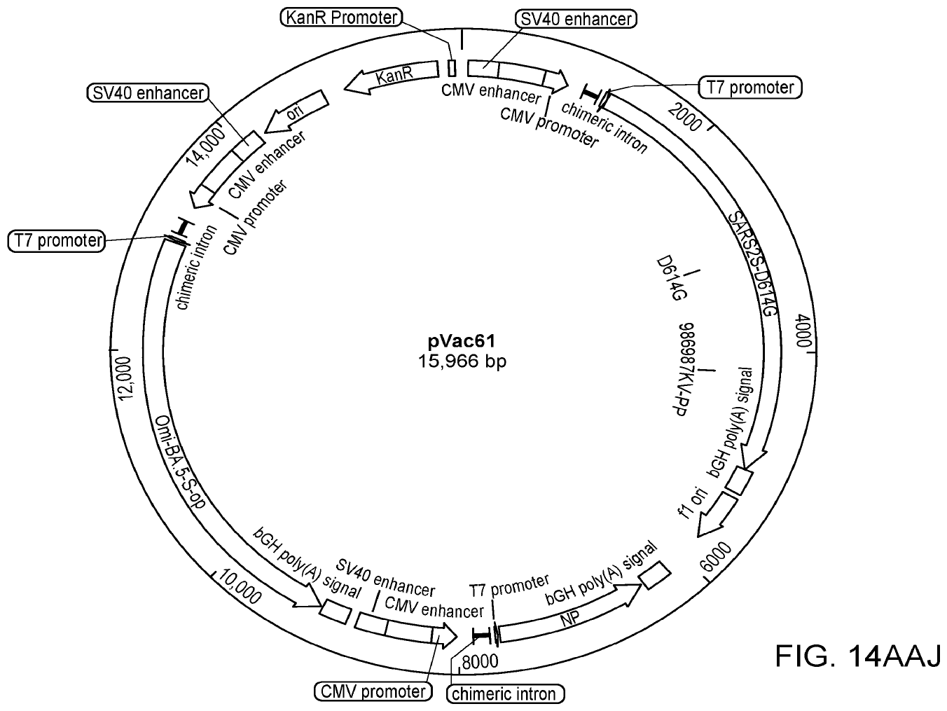

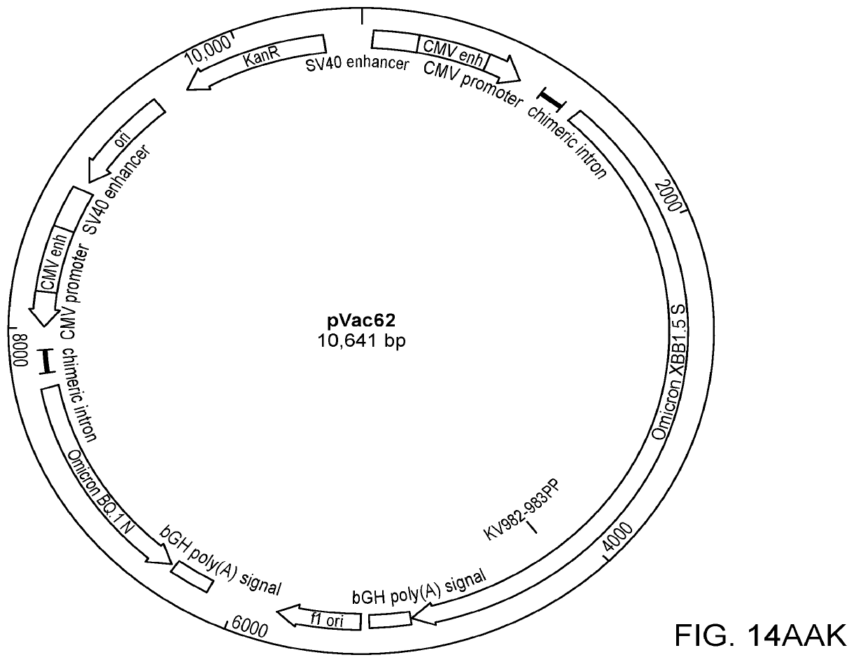

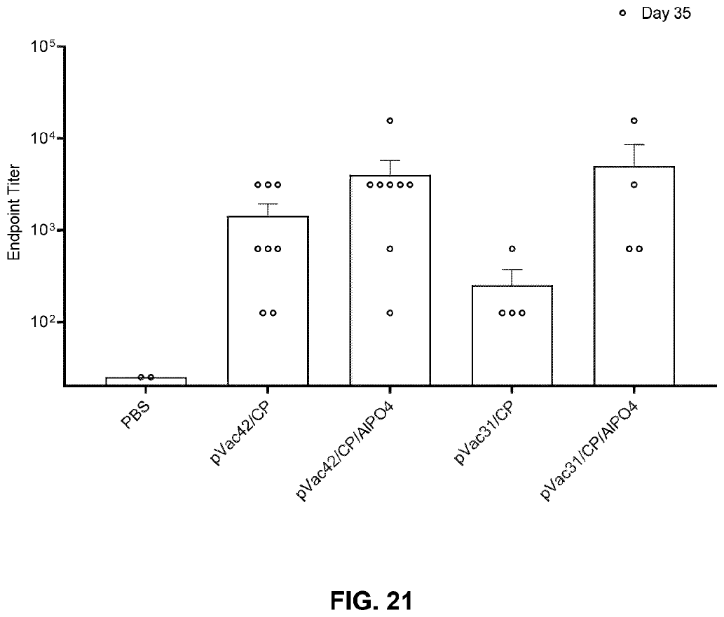

Plasmid vectors including pVac42 encoding the SARS-CoV-2 Spike Protein with the 2P modification (single antigen) and pVac31 encoding the SARS-CoV-2 Spike Protein with D614G mutation and the 2P modification were administered at 125 μg plasmid DNA with 0.5% crown poloxamer or with 0.5% crown poloxamer and 0.5 mg/mL aluminum P04 adjuvant by intramuscular injection (i.m.) at day 0 (prime) and day 14 (boost) to Balb/C mice. PBS was administered as a control. IgG titers were determined 35 days after administration and showed increased induction of IgG with pVac42 and pVac31 administered with 0.5% crown poloxamer and 0.5 mg/mL aluminum P04 adjuvant compared to 0.5% crown poloxamer alone (FIG. 21).

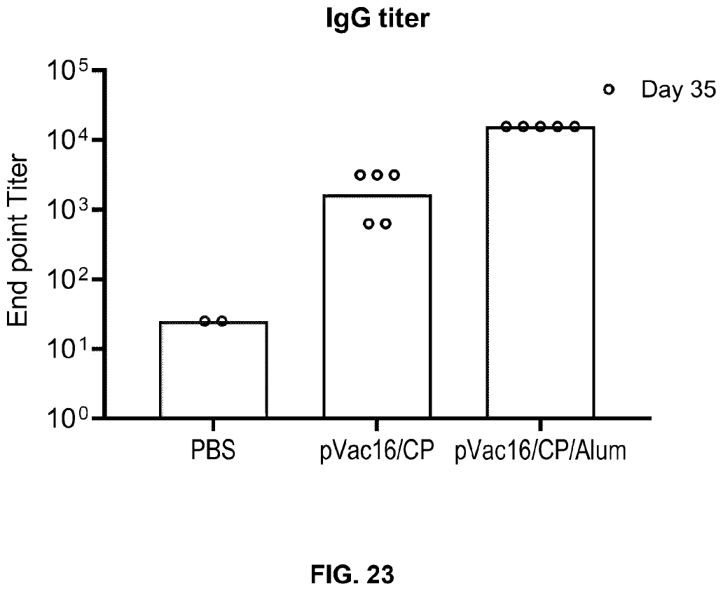

The plasmid vector pVac16 encoding full length SARS-CoV-2 delta variant Spike Protein with the 2P modification was administered at 125 μg plasmid DNA with 0.5% crown poloxamer or with 0.5% crown poloxamer and 0.5 mg/mL aluminum P04 adjuvant by intramuscular injection (i.m.) at day 0 (prime) and day 14 (boost) to Balb/C mice. IgG titers were determined 35 days after administration and showed increased induction of IgG after administration of pVac16 (FIG. 23). pVac16 with crown poloxamer and aluminum P04 adjuvant elicited a higher IgG titer compared to pVac16 with crown poloxamer alone.

### II. Neutralizing Activity of a Multi-Antigen Single Plasmid Vaccine Against Multiple Viral Strains

Plasmid pVac-17 encoding the CMV-Spike-2P antigen D614G+Delta variants of SARS-CoV-2 (dual antigen) were administered at 125 μg plasmid DNA with 0.5% crown poloxamer and aluminum P04 adjuvant i.m. at day 0 (prime) and day 14 (boost) to BalbC mice. SARS-CoV-2 D614-2P mRNA was administered as control. Virus neutralizing activity (IC50 titer) was determined on day 35 after prime in a pseudoviral challenge assay using D614G Strain (FIG. 16B, left graph) and Delta Strain (FIG. 16B, right graph). Both, pVac-17 and the mRNA control elicited neutralizing activity against the D614G strain indicated by high IC50 titers. However, only pVac-17 elicited similarly high neutralizing activity against the Delta Strain, wherein a drop in titer was observed with the mRNA control indicating reduced neutralizing activity of the mRNA control.

### III. Neutralizing Activity of Two Single Antigen Plasmid Vaccines

Plasmid vectors pVac-15 encoding the CMV-Spike-2P antigen of D614G variant of SARS-CoV-2 (single antigen) and pVac-16 encoding the CMV-Spike-2P antigen of Delta variant of SARS-CoV-2 (single antigen) were administered at 125 μg plasmid DNA with 0.5% crown poloxamer and aluminum P04 adjuvant i.m. at day 0 (prime) and day 14 (boost) to BalbC mice. Virus neutralizing activity (IC50 titer) was determined on day 35 after prime in a pseudoviral challenge assay using D614G Strain (FIG. 16C, left graph) and Delta Strain (FIG. 16C, right graph). Both, pVac-15 and pVac-16 elicited neutralizing activity against the D614G strain indicated by high IC50 titers. However, pVac-16 elicited significantly higher neutralizing activity against the Delta Strain compared to pVac-15 indicating efficient neutralizing activity of the pVac-16 against its respective antigen strain.

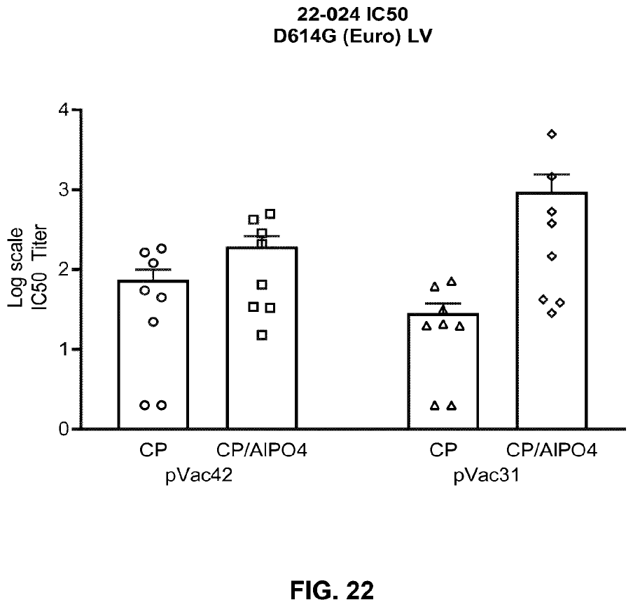

Plasmid vectors including pVac42 encoding the SARS-CoV-2 Spike Protein with the 2P modification and pVac31 encoding the SARS-CoV-2 Spike Protein with D614G mutation and the 2P modification were administered at 125 μg plasmid DNA with 0.5% crown poloxamer or 0.5% crown poloxamer and 0.5 mg/mL aluminum P04 adjuvant by intramuscular injection (i.m.) at day 0 and day 14 to Balb/C mice. Virus neutralizing activity (IC50 titer) was determined on day 35 after prime in a pseudoviral challenge assay using D614G Strain. Both, pVac42 and pVac31 elicited neutralizing activity against the D614G strain indicated by high IC50 titers (FIG. 22). Both, pVac42 and pVac31 elicited higher neutralizing activity when administered with crown poloxamer and aluminum P04 adjuvant compared to crown poloxamer alone.

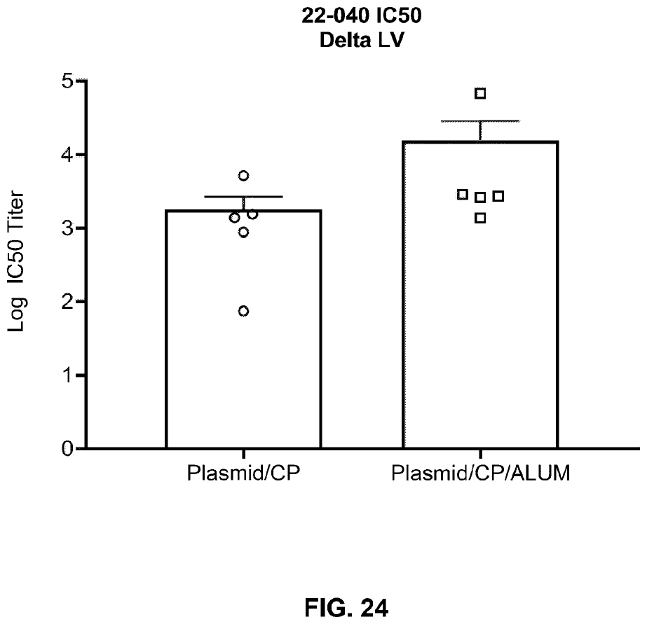

The plasmid vector pVac16 encoding the full length SARS-CoV-2 delta variant Spike Protein with the 2P modification was administered at 125 μg plasmid DNA with 0.5% crown poloxamer or with 0.5% crown poloxamer and 0.5 mg/mL aluminum P04 adjuvant by intramuscular injection (i.m.) at day 0 and day 21 to Balb/C mice. Virus neutralizing activity (IC50 titer) was determined on day 42 after prime in a pseudoviral challenge assay using Delta Strain. pVac16 formulated with crown poloxamer and aluminum PO₄ adjuvant elicted higher neutralizing activity compared to crown poloxamer alone (FIG. 24).

IV. EF1 Promoter Based Vectors in Combination with Crown Poloxamer and Aluminum Adjuvant and STING agonist

Plasmid vector pVac-9 encoding the full length SARS-CoV-2 D614G variant spike protein with 2P modification (single antigen) was administered at 125 μg plasmid DNA with crown poloxamer and aluminum P04 adjuvant or STING agonist i.m. at day 0 (prime) and day 14 (boost) to BalbC mice. PBS was administered as control. IFN-7 production per 1 Mil. Spleen cells was measured on day 35 after prime and demonstrated improved vaccine immunogenicity by aluminum adjuvant and STING agonist (FIG. 17A). IgG titers were also determined on day 35 after prime and demonstrated increases in IgG titers with aluminum adjuvant and STING agonist (FIG. 17B).

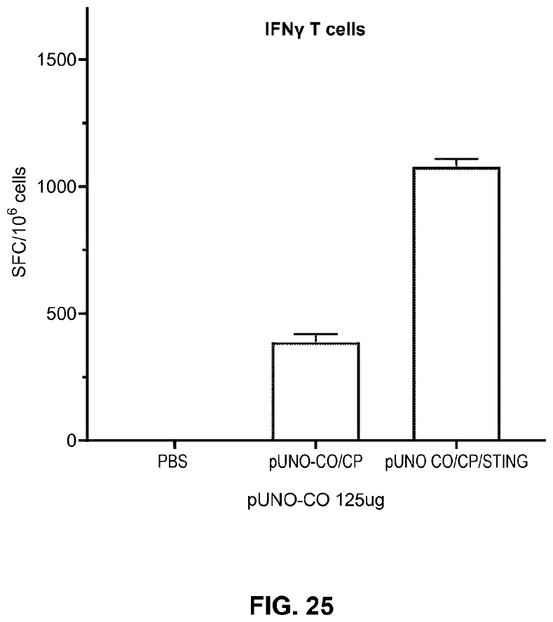

The plasmid vector pUNO encoding the Spike protein was administered at 125 μg plasmid DNA with 0.5% crown poloxamer or with 0.5% crown poloxamer and STING agonist by intramuscular injection (i.m.) at day 0 (prime) and day 14 (boost) to Balb/C mice. PBS was administered as control. IFN-7 production per 1 Mil. Spleen cells was measured on day 35 after immunization and demonstrated improved vaccine immunogenicity by crown poloxamer and STING agonist compared to crown poloxamer alone (FIG. 25).

V. EF1 Promoter Based Vectors in Combination with Crown Poloxamer

Plasmid vector pVac-9 encoding the full length SARS-CoV-2 D614G variant spike protein with 2P modification (single antigen) was administered at 250 μg plasmid DNA with 0.5% crown poloxamer i.m. at day 0 (prime) and day 14 (boost) to BalbC mice. PBS was administered as control. IgG titers determined on days 14 and 35 after prime demonstrated increases in IgG titers at day 35 compared to day 14 with pVac-9 in combination with crown poloxamer (FIG. 18 A, 2) compared to pVac-9 naked plasmid DNA only (FIG. 18A, 3). IFN-7 production per 1 Mio. Spleen cells as measured on day 35 after prime demonstrated improved vaccine immunogenicity of the plasmid DNA crown poloxamer combination (FIG. 18B, 2) over naked plasmid DNA (FIG. 18B, 3).

VI. H₁N1 Vector in Combination with Crown Poloxamer

Plasmid vector pH1N1 encoding the H₁N1 HA Brisbane 2007 antigen was administered at 125 μg plasmid DNA with crown poloxamer, or crown poloxamer and aluminum P04 adjuvant i.m. at day 0 (prime) and day 14 (boost) to BalbC mice. PBS was administered as control. IgG titers were determined on day 14 and 35 after prime and demonstrated increases in IgG titers with crown poloxamer and crown poloxamer and aluminum adjuvant on day 14 and more pronounced on day 35 after prime (FIG. 19).

### VII. Body Weight

BALB/c mice immunized with pUNO-Spike vector (C2) alone or in combination with 0.5% crown poloxamer (C2/F1), 0.5% crown poloxamer and 10 μg STING agonist (C2/F2), or 0.5% crown poloxamer and 50 μg aluminum P04 (C2/F3) were weighed during the first three days after immunization and after the first booster immunization (days 14-16). The weight of BALB/c mice immunized with 10⁸ viral particles (VIRAL), g of spike protein (PROTEIN) and mRNA/2P control was also determined, FIG. 20 shows that the weights of all treated mice did not differ substantially from the weight of control mice (FIG. 20).

VIII. CMV Promoter Based Vectors in Combination with Crown Poloxamer and Sting Agonist

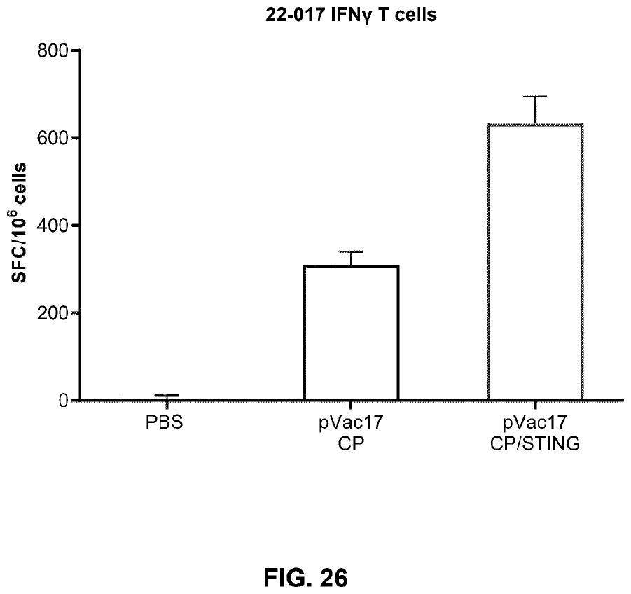

The plasmid vector pVac17 encoding the full length SARS-CoV-2 D614G variant Spike Protein with the 2P modification and expressing the SARS-CoV-2 full length delta variant Spike Protein with the 2P modification was administered at 125 μg plasmid DNA with 0.5% crown poloxamer or with 0.5% crown poloxamer and STING agonist by intramuscular injection (i.m.) at day 0 (prime) and day 14 to Balb/C mice. PBS was administered as control. IFN-7 production per 1 Mil. spleen cells was measured on day 35 after immunization and demonstrated improved vaccine immunogenicity by crown poloxamer and STING agonist compared to crown poloxamer alone (FIG. 26).

### Example 9: In Vivo Testing of Compositions Comprising Crown Poloxamer and/or Adjuvant in Mice

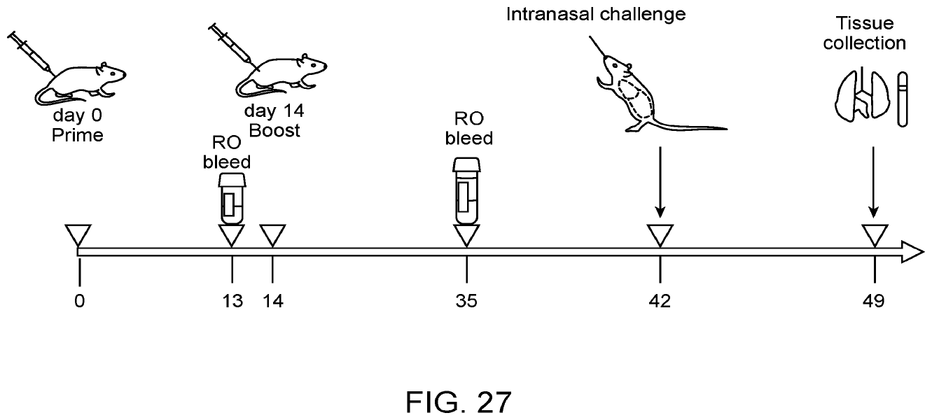

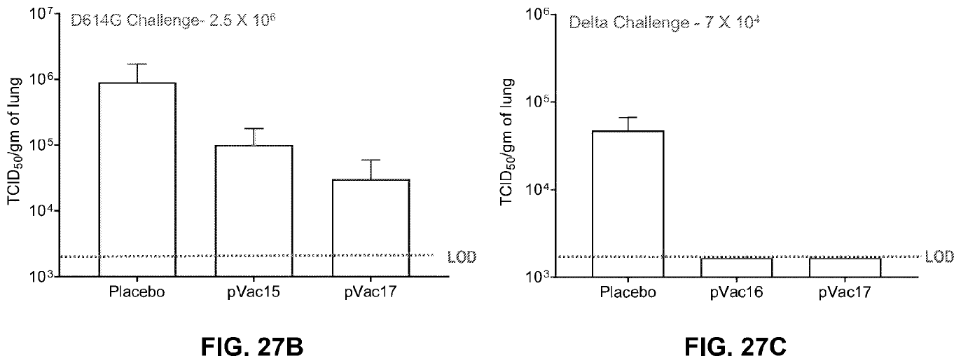

The plasmid vectors pVac15 encoding the full length SARS-CoV-2 Spike Protein with the 2P modification, pVac16 encoding the full length SARS-CoV-2 delta variant Spike Protein with the 2P modification, and pVac17 encoding the full length SARS-CoV-2 Spike Protein with the 2P modification and the SARS-CoV-2 full length delta variant Spike Protein with the 2P modification, were administered at 125 μg plasmid DNA with 0.5% crown poloxamer and 0.5 mg/mL aluminum P04 adjuvant by intramuscular injection (i.m.) to hACE2:K18 mice at day 0 (prime) and day 14 (boost). Bleeds were taken at days 13 and 35. Mice were challenged intranasally at day 42 and tissue was collected 7 days after challenge (day 49). A schematic of the dosing schedule is shown in FIG. 27A.

The ability of pVac15, pVac16, pVac17, or placebo to provide protection after challenge with D614G strain or Delta strain was assessed by a tissue culture infectious dose (TCID₅₀) assay. pVac15 and pVac17 were protective against challenge with the D614G strain (FIG. 27B) and pVac16 and pVac17 were protective against challenge with the Delta strain (FIG. 27C).

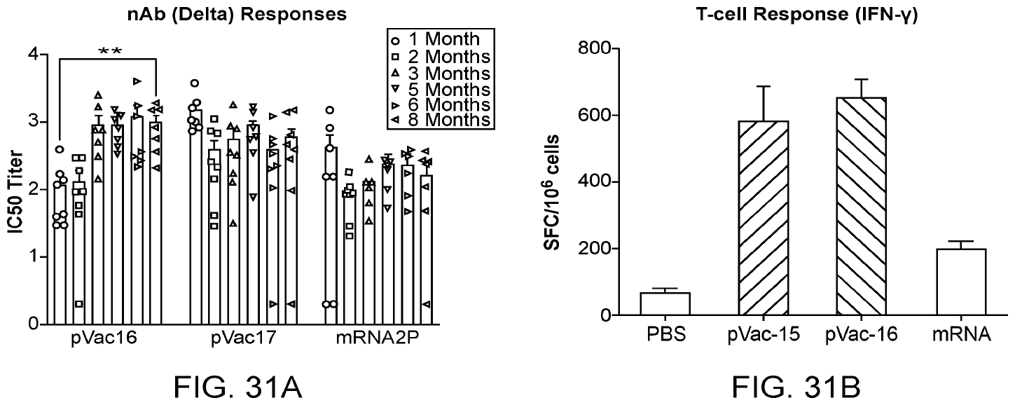

The long-term durability of the immunization was assessed by analyzing neutralizing antibody titers and IFN-7 production. Virus neutralizing activity (IC50 titer) was determined 1, 2, 3, 5, 6, and 8 months after prime in a pseudoviral challenge assay. pVac16 and pVac17 elicited a neutralizing activity through 8 months post-immunization (FIG. 31A). pVac16 showed significantly higher neutralizing activity 8 months post-immunization compared to 1 month post-immunization. IFN-7 production per 1 Mil. spleen cells was measured 12 months after immunization and long-term vaccine immunogenicity of pVac15 and pVac16 (FIG. 31B). 12 months after immunization, pVac15 and pVac16 elicited a higher level IFN-7 production per 1 Mil. spleen cells compared to control mRNA vaccine.

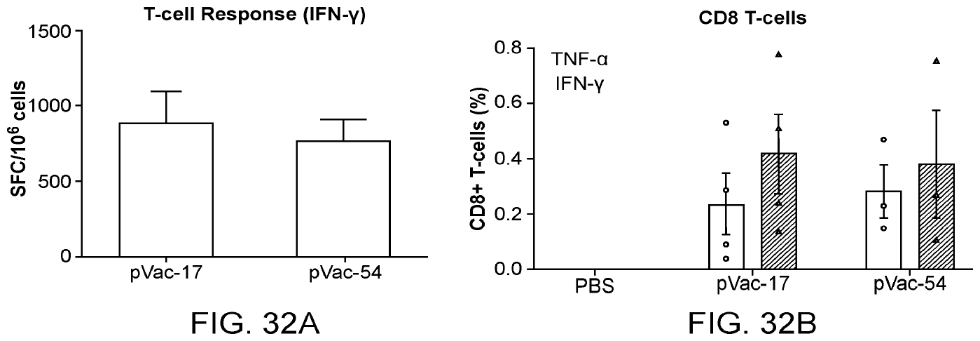

The plasmid vectors pVac17 encoding the full length SARS-CoV-2 Spike Protein with the 2P modification and the SARS-CoV-2 full length delta variant Spike Protein with the 2P modification and pVac54 encoding the full length SARS-CoV-2 Spike Protein with D614G mutation and the 2P modification driven by a CMV promoter with the aid of a SV40 enhancer sequence and expressing the full length SARS-CoV-2 delta variant Spike Protein with the 2P modification were administered at 125 g plasmid DNA with 0.5% crown poloxamer and 0.5 mg/mL aluminum P04 adjuvant by intramuscular injection (i.m.) to Balb/c mice mice. PBS was administered as a control. Mice were challenged with pseudotyped lentivirus and IFN-7 production in spleen cells and the percent of CD8+ cells expressing IFN-7 and TNF-α was assessed. Immunization of pVac17 and pVac54 elicted IFN-7 production in spleenocytes (FIG. 32A) and increased the population of CD8+ T cells in spleenocytes (FIG. 32B).

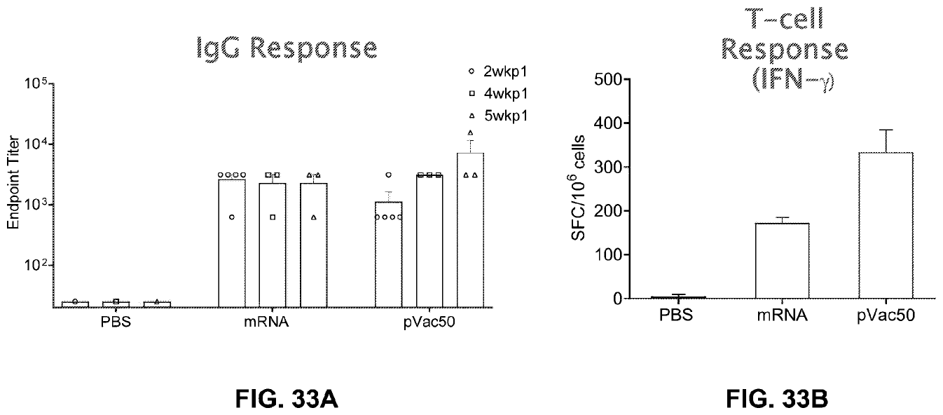

The plasmid vector pVac50 encoding the full length SARS-CoV-2 delta variant Spike Protein with the 2P modification was administered a single dose of 125 g plasmid DNA with 0.5% crown poloxamer and 0.5 mg/mL aluminum P04 adjuvant by intramuscular injection (i.m.) to mice. PBS and an mRNA vaccine were administered as controls. IgG titers were determined at 2wkp1, 4wkp1, and 5wkp1 and showed an induction of IgG after immunization with pVac50 (FIG. 33A). IFN-7 production per 1 Mil. spleen cells was measured and demonstrated improved vaccine immunogenicity by immunization with pVac50 compared to immunization with the mRNA vaccine (FIG. 33B).

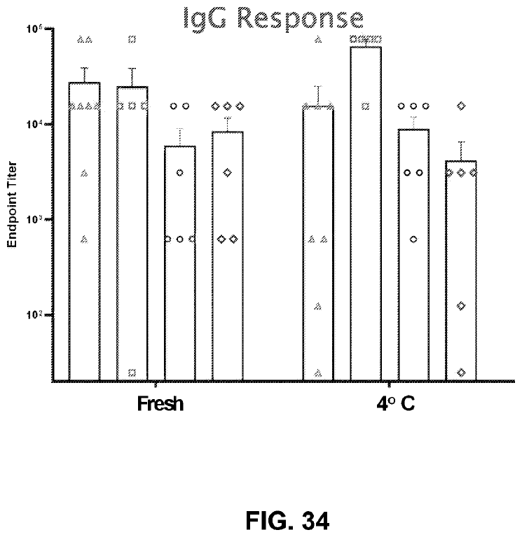

Either freshly prepared pVac17 or pVac17 that was stored at 4° C. for 12 months was administered at 125 g with 0.5% crown poloxamer and 0.5 mg/mL aluminum P04 adjuvant by intramuscular injection (i.m.) to mice. IgG titers showed that pVac17 was stable at 4° C. for at least 9 months (FIG. 34).

### Example 10: In Vivo Testing of Compositions Comprising Crown Poloxamer and/or Adjuvant in Cynomolgus Monkeys

The plasmid vectors pVac15 encoding the full length SARS-CoV-2 Spike Protein with the 2P modification, pVac16 encoding the full length SARS-CoV-2 delta variant Spike Protein with the 2P modification, or a combination of pVac15 and pVac16 were administered at 1 mg (pVac15 alone), 2 mg (pVac16 alone), or 5 mg (pVac15)+1 mg (pVac16) to Cynomolgus monkeys at day 0 (prime), day 28 (boost), and day 84 (boost). Monkeys administered the combination of pVac15 and pVac16 were administered 2 mg pVac16 at the day 84 second boost. All plasmid vectors were formulated with 0.5% crown poloxamer and 0.5 mg/mL aluminum P04 adjuvant. Placebo and 100 g of a mRNA vaccine formulated with a LNP were administered as controls.

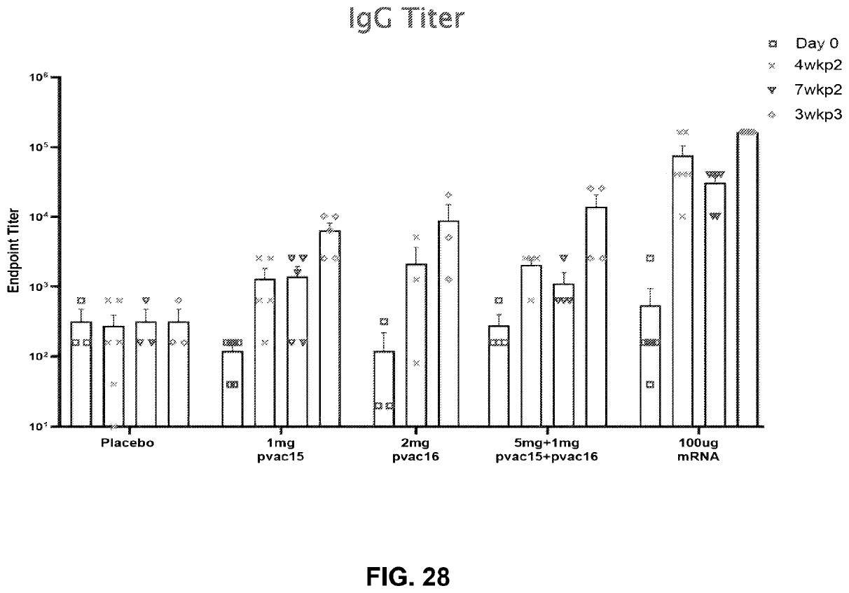

IgG titer was assessed at 4wkp2, 7wkp2, and 3wkp3 after day 0. Immunization with pVac15 alone, pVac16 alone, or a combination of pVac15 and pVac16 induced an increase in IgG titer (FIG. 28).

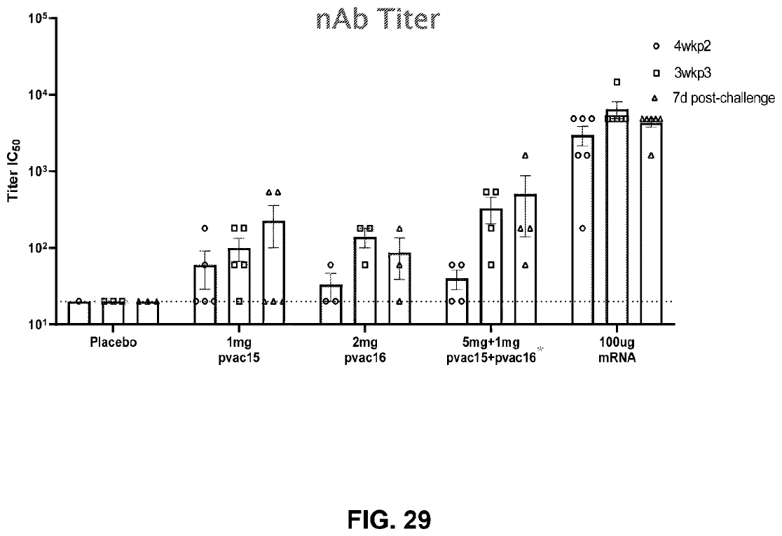

Cynomolgus monkeys were challenged with SARS-CoV-2 D614G variant at 1×10⁶ TCID₅₀ on day 98 and virus neutralizing activity (IC50 titer) was assessed on 4wkp2, 3wkp3, and 7 days post-challenge. pVac15 alone, pVac16 alone, and the combination of pVac15 and pVac16 elicited neutralizing activity against the challenged strain (FIG. 29). 90% of the immunized subjects showed a neutralizing antibody response.

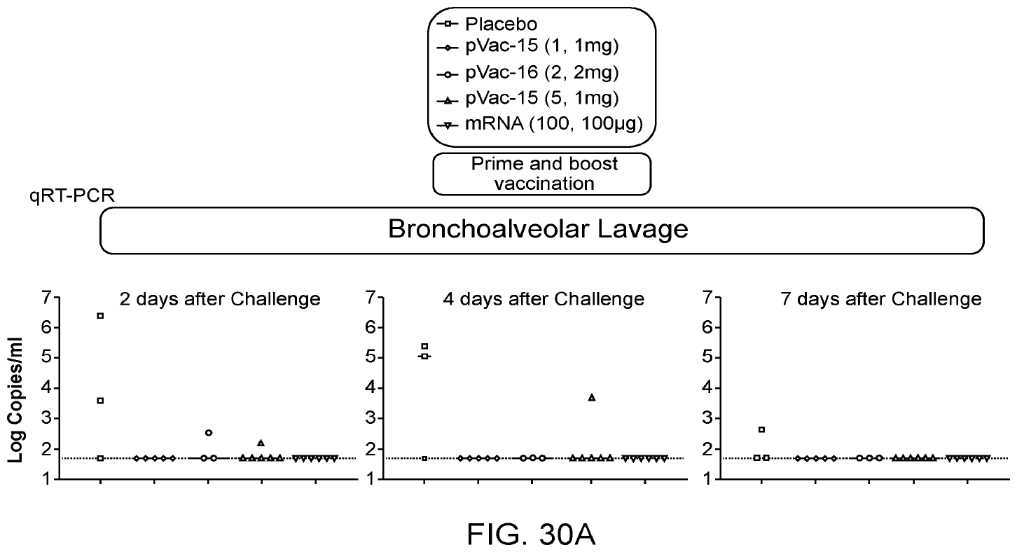

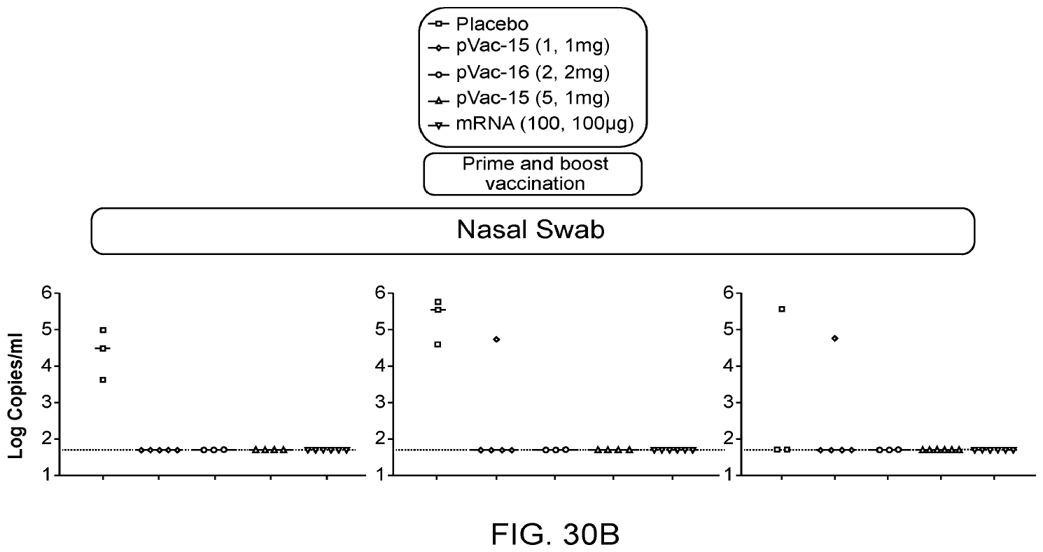

mRNA levels of the challenged virus in the lung (FIG. 30A) and nasal cavity (FIG. 30B) was assessed 2, 4, and 7 days post-challenge. Nearly all of the monkeys immunized with pVac15 alone, pVac16 alone, or a combination of pVac15 and pVac16 did not express detectable levels of challenged virus mRNA (Table 3). Immunization with pVac15 alone, pVac16 alone, or a combination of pVac15 and pVac16 showed comparable efficacy to the mRNA vaccine control.

TABLE 3
mRNA Levels of Challenged Virus

| | Bronchoalveolar Lavage | | | Nasal Swab | | |
|---|---|---|---|---|---|---|
| Vaccine | Day 2 | Day 4 | Day 7 | Day 2 | Day 4 | Day 7 |
| Placebo | 6.20 | 4.37 | 3.70 | 5.37 | 4.70 | 4.20 |
| | 3.20 | 4.37 | <2.7 | 6.37 | 5.20 | 3.70 |
| | 5.20 | <2.7 | <2.7 | 5.20 | 5.70 | 3.37 |
| pVac15 | <2.7 | <2.7 | <2.7 | <2.7 | <2.7 | <2.7 |
| (1 mg) | <2.7 | <2.7 | <2.7 | <2.7 | <2.7 | <2.7 |
| | <2.7 | <2.7 | <2.7 | <2.7 | 4.20 | <2.7 |
| | <2.7 | <2.7 | <2.7 | 4.70 | 5.37 | <2.7 |
| | <2.7 | <2.7 | <2.7 | <2.7 | <2.7 | <2.7 |
| pVac16 | <2.7 | <2.7 | <2.7 | <2.7 | <2.7 | <2.7 |
| (2 mg) | <2.7 | <2.7 | <2.7 | <2.7 | <2.7 | <2.7 |
| | <2.7 | <2.7 | <2.7 | <2.7 | <2.7 | <2.7 |
| pVac15/ | <2.7 | <2.7 | <2.7 | <2.7 | <2.7 | <2.7 |
| pVac16 | <2.7 | <2.7 | <2.7 | <2.7 | <2.7 | <2.7 |
| (5 mg, | <2.7 | <2.7 | <2.7 | <2.7 | <2.7 | <2.7 |
| 1 mg) | <2.7 | <2.7 | <2.7 | <2.7 | <2.7 | <2.7 |
| mRNA | <2.7 | <2.7 | <2.7 | <2.7 | <2.7 | <2.7 |
| (100 | <2.7 | <2.7 | <2.7 | <2.7 | <2.7 | <2.7 |
| μg) | <2.7 | <2.7 | <2.7 | <2.7 | <2.7 | <2.7 |
| | <2.7 | <2.7 | <2.7 | <2.7 | <2.7 | <2.7 |
| | <2.7 | <2.7 | <2.7 | <2.7 | <2.7 | <2.7 |
| | <2.7 | <2.7 | <2.7 | <2.7 | <2.7 | <2.7 |

<2.7 means below the lower limit of detection.

TABLE 4
Sequences

| | |
|---|---|
| SEQ ID NO: 1 | ATGTTTGTTTTTCTTGTTTTATTGCCACTAGTCTCTAGTCAGT |
| SARS-CoV-2 S | GTGTTAATCTTACAACCAGAACTCAATTACCCCCTGCATACA |
| Protein (codon | CTAATTCTTTCACACGTGGTGTTTATTACCCTGACAAAGTTTT |
| optimized | CAGATCCTCAGTTTTACATTCAACTCAGGACTTGTTCTTACCT |
| nucleic acid | TTCTTTTCCAATGTTACTTGGTTCCATGCTATACATGTCTCTG |
| sequence) | GGACCAATGGTACTAAGAGGTTTGATAACCCTGTCCTACCAT |
| | TTAATGATGGTGTTTATTTTGCTTCCACTGAGAAGTCTAACA |
| | TAATAAGAGGCTGGATTTTTGGTACTACTTTAGATTCGAAGA |
| | CCCAGTCCCTACTTATTGTTAATAACGCTACTAATGTTGTTAT |
| | TAAAGTCTGTGAATTTCAATTTTGTAATGATCCATTTTTGGGT |
| | GTTTATTACCACAAAAACAACAAAAGTTGGATGGAAAGTGA |
| | GTTCAGAGTTTATTCTAGTGCGAATAATTGCACTTTTGAATA |
| | TGTCTCTCAGCCTTTTCTTATGGACCTTGAAGGAAAACAGGG |
| | TAATTTCAAAAATCTTAGGGAATTTGTGTTTAAGAATATTGA |
| | TGGTTATTTTAAAATATATTCTAAGCACACGCCTATTAATTT |
| | AGTGCGTGATCTCCCTCAGGGTTTTTCGGCTTTAGAACCATT |
| | GGTAGATTTGCCAATAGGTATTAACATCACTAGGTTTCAAAC |
| | TTTACTTGCTTTACATAGAAGTTATTTGACTCCTGGTGATTCT |
| | TCTTCAGGTTGGACAGCTGGTGCTGCAGCTTATTATGTGGGT |
| | TATCTTCAACCTAGGACTTTTCTATTAAAATATAATGAAAAT |
| | GGAACCATTACAGATGCTGTAGACTGTGCACTTGACCCTCTC |
| | TCAGAAACAAAGTGTACGTTGAAATCCTTCACTGTAGAAAA |
| | AGGAATCTATCAAACTTCTAACTTTAGAGTCCAACCAACAGA |
| | ATCTATTGTTAGATTTCCTAATATTACAAACTTGTGCCCTTTT |
| | GGTGAAGTTTTTAACGCCACCAGATTTGCATCTGTTTATGCT |
| | TGGAACAGGAAGAGAATCAGCAACTGTGTTGCTGATTATTCT |
| | GTCCTATATAATTCCGCATCATTTTCCACTTTTAAGTGTTATG |
| | GAGTGTCTCCTACTAAATTAAATGATCTCTGCTTTACTAATG |
| | TCTATGCAGATTCATTTGTAATTAGAGGTGATGAAGTCAGAC |
| | AAATCGCTCCAGGGCAAACTGGAAAGATTGCTGATTATAAT |
| | TATAAATTACCAGATGATTTTACAGGCTGCGTTATAGCTTGG |
| | AATTCTAACAATCTTGATTCTAAGGTTGGTGGTAATTATAAT |
| | TACCTGTATAGATTGTTTAGGAAGTCTAATCTCAAACCTTTT |
| | GAGAGAGATATTTCAACTGAAATCTATCAGGCCGGTAGCAC |
| | ACCTTGTAATGGTGTTGAAGGTTTTAATTGTTACTTTCCTTTA |
| | CAATCATATGGTTTCCAACCCACTAATGGTGTTGGTTACCAA |
| | CCATACAGAGTAGTAGTACTTTCTTTTGAACTTCTACATGCA |
| | CCAGCAACTGTTTGTGGACCTAAAAAGTCTACTAATTTGGTT |
| | AAAAACAAATGTGTCAATTTCAACTTCAATGGTTTAACAGGC |
| | ACAGGTGTTCTTACTGAGTCTAACAAAAAGTTTCTGCCTTTC |
| | CAACAATTTGGCAGAGACATTGCTGACACTACTGATGCTGTC |
| | CGTGATCCACAGACACTTGAGATTCTTGACATTACACCATGT |
| | TCTTTTGGTGGTGTCAGTGTTATAACACCAGGAACAAATACT |
| | TCTAACCAGGTTGCTGTTCTTTATCAGGATGTTAACTGCACA |
| | GAAGTCCCTGTTGCTATTCATGCAGATCAACTTACTCCTACT |
| | TGGCGTGTTTATTCTACAGGTTCTAATGTTTTTCAAACACGTG |
| | CAGGCTGTTTAATAGGGGCTGAACATGTCAACAACTCATATG |
| | AGTGTGACATACCCATTGGTGCAGGTATATGCGCTAGTTATC |
| | AGACTCAGACTAATTCTCCTCGGCGGGCACGTAGTGTAGCTA |
| | GTCAATCCATCATTGCCTACACTATGTCACTTGGTGCAGAAA |
| | ATTCAGTTGCTTACTCTAATAACTCTATTGCCATACCCACAA |
| | ATTTTACTATTAGTGTTACCACAGAAATTCTACCAGTGTCTA |
| | TGACCAAGACATCAGTAGATTGTACAATGTACATTTGTGGTG |
| | ATTCAACTGAATGCAGCAATCTTTTGTTGCAATATGGCAGTT |
| | TTTGTACACAATTAAACCGTGCTTTAACTGGAATAGCTGTTG |
| | AACAAGACAAAAACACCCAAGAAGTTTTTGCACAAGTCAAA |
| | CAAATTTACAAAACACCACCAATTAAAGATTTTGGTGGTTTT |
| | AATTTTTCACAAATATTACCAGATCCATCAAAACCAAGCAAG |
| | AGGTCATTTATTGAAGATCTACTTTTCAACAAAGTGACACTT |
| | GCAGATGCTGGCTTCATCAAACAATATGGTGATTGCCTTGGT |
| | GATATTGCTGCTAGAGACCTCATTTGTGCACAAAAGTTTAAC |
| | GGCCTTACTGTTTTGCCACCTTTGCTCACAGATGAAATGATT |
| | GCTCAATACACTTCTGCACTGTTAGCGGGTACAATCACTTCT |
| | GGTTGGACCTTTGGTGCAGGTGCTGCATTACAAATACCATTT |
| | GCTATGCAAATGGCTTATAGGTTTAATGGTATTGGAGTTACA |
| | CAGAATGTTCTCTATGAGAACCAAAAATTGATTGCCAACCA |
| | ATTTAATAGTGCTATTGGCAAAATTCAAGACTCACTTTCTTC |
| | CACAGCAAGTGCACTTGGAAAACTTCAAGATGTGGTCAACC |
| | AAAATGCACAAGCTTTAAACACGCTTGTTAAACAACTTAGCT |
| | CCAATTTTGGTGCAATTTCAAGTGTTTTAAATGATATCCTTTC |
| | ACGTCTTGACAAAGTTGAGGCTGAAGTGCAAATTGATAGGT |
| | TGATCACAGGCAGACTTCAAAGTTTGCAGACATATGTGACTC |
| | AACAATTAATTAGAGCTGCAGAAATCAGAGCTTCTGCTAATC |
| | TTGCTGCTACTAAAATGTCAGAGTGTGTACTTGGACAATCAA |
| | AAAGAGTTGATTTTTGTGGAAAGGGCTATCATCTTATGTCCT |
| | TCCCTCAGTCAGCACCTCATGGTGTAGTCTTCTTGCATGTGA |
| | CTTATGTCCCTGCACAAGAAAAGAACTTCACAACTGCTCCTG |
| | CCATTTGTCATGATGGAAAAGCACACTTTCCTCGTGAAGGTG |
| | TCTTTGTTTCAAATGGCACACACTGGTTTGTAACACAAAGGA |
| | ATTTTTATGAACCACAAATCATTACTACAGACAACACATTTG |
| | TGTCTGGTAACTGTGATGTTGTAATAGGAATTGTCAACAACA |
| | CAGTTTATGATCCTTTGCAACCTGAATTAGACTCATTCAAGG |
| | AGGAGTTAGATAAATATTTTAAGAATCATACATCACCAGAT |
| | GTTGATTTAGGTGACATCTCTGGCATTAATGCTTCAGTTGTA |
| | AACATTCAAAAAGAAATTGACCGCCTCAATGAGGTTGCCAA |
| | GAATTTAAATGAATCTCTCATCGATCTCCAAGAACTTGGAAA |
| | GTATGAGCAGTATATAAAATGGCCATGGTACATTTGGCTAG |
| | GTTTTATAGCTGGCTTGATTGCCATAGTAATGGTGACAATTA |
| | TGCTTTGCTGTATGACCAGTTGCTGTAGTTGTCTCAAGGGCT |
| | GTTGTTCTTGTGGATCCTGCTGCAAATTTGATGAAGACGACT |
| | CTGAGCCAGTGCTCAAAGGAGTCAAATTACATTACACATAA |
| SEQ ID NO: 2 | MFVFLVLLPLVSSQCVNLTTRTQLPPAYTNSFTRGVYYPDKVFR |
| SARS-CoV-2 S | SSVLHSTQDLFLPFFSNVTWFHAIHVSGTNGTKRFDNPVLPFND |
| Protein (amino | GVYFASTEKSNIIRGWIFGTTLDSKTQSLLIVNNATNVVIKVCEF |
| acid sequence) | QFCNDPFLGVYYHKNNKSWMESEFRVYSSANNCTFEYVSQPFL |
| | MDLEGKQGNFKNLREFVFKNIDGYFKIYSKHTPINLVRDLPQGF |
| | SALEPLVDLPIGINITRFQTLLALHRSYLTPGDSSSGWTAGAAAY |
| | YVGYLQPRTFLLKYNENGTITDAVDCALDPLSETKCTLKSFTVE |
| | KGIYQTSNFRVQPTESIVRFPNITNLCPFGEVENATRFASVYAW |
| | NRKRISNCVADYSVLYNSASFSTFKCYGVSPTKLNDLCFTNVY |
| | ADSFVIRGDEVRQIAPGQTGKIADYNYKLPDDFTGCVIAWNSN |
| | NLDSKVGGNYNYLYRLFRKSNLKPFERDISTEIYQAGSTPCNGV |
| | EGFNCYFPLQSYGFQPTNGVGYQPYRVVVLSFELLHAPATVCG |
| | PKKSTNLVKNKCVNFNFNGLTGTGVLTESNKKFLPFQQFGRDI |
| | ADTTDAVRDPQTLEILDITPCSFGGVSVITPGTNTSNQVAVLYQ |
| | DVNCTEVPVAIHADQLTPTWRVYSTGSNVFQTRAGCLIGAEHV |
| | NNSYECDIPIGAGICASYQTQTNSPRRARSVASQSIIAYTMSLGA |
| | ENSVAYSNNSIAIPTNFTISVTTEILPVSMTKTSVDCTMYICGDST |
| | ECSNLLLQYGSFCTQLNRALTGIAVEQDKNTQEVFAQVKQIYK |
| | TPPIKDFGGFNFSQILPDPSKPSKRSFIEDLLFNKVTLADAGFIKQ |
| | YGDCLGDIAARDLICAQKFNGLTVLPPLLTDEMIAQYTSALLAG |
| | TITSGWTFGAGAALQIPFAMQMAYRENGIGVTQNVLYENQKLI |
| | ANQFNSAIGKIQDSLSSTASALGKLQDVVNQNAQALNTLVKQL |
| | SSNFGAISSVLNDILSRLDKVEAEVQIDRLITGRLQSLQTYVTQQ |
| | LIRAAEIRASANLAATKMSECVLGQSKRVDFCGKGYHLMSFPQ |
| | SAPHGVVFLHVTYVPAQEKNFTTAPAICHDGKAHFPREGVFVS |
| | NGTHWFVTQRNFYEPQIITTDNTFVSGNCDVVIGIVNNTVYDPL |
| | QPELDSFKEELDKYFKNHTSPDVDLGDISGINASVVNIQKEIDRL |
| | NEVAKNLNESLIDLQELGKYEQYIKWPWYIWLGFIAGLIAIVMV |
| | TIMLCCMTSCCSCLKGCCSCGSCCKFDEDDSEPVLKGVKLHYT |
| SEQ ID NO: 3 | ATGTTTGTTTTTCTTGTTTTATTGCCACTAGTCTCTAGTCAGT |
| SARS-CoV-2 | GTGTTAATCTTACAACCAGAACTCAATTACCCCCTGCATACA |
| D614G S Protein | CTAATTCTTTCACACGTGGTGTTTATTACCCTGACAAAGTTTT |
| (codon | CAGATCCTCAGTTTTACATTCAACTCAGGACTTGTTCTTACCT |
| optimized | TTCTTTTCCAATGTTACTTGGTTCCATGCTATACATGTCTCTG |
| nucleic acid | GGACCAATGGTACTAAGAGGTTTGATAACCCTGTCCTACCAT |
| sequence) | TTAATGATGGTGTTTATTTTGCTTCCACTGAGAAGTCTAACA |
| | TAATAAGAGGCTGGATTTTTGGTACTACTTTAGATTCGAAGA |
| | CCCAGTCCCTACTTATTGTTAATAACGCTACTAATGTTGTTAT |
| | TAAAGTCTGTGAATTTCAATTTTGTAATGATCCATTTTTGGGT |
| | GTTTATTACCACAAAAACAACAAAAGTTGGATGGAAAGTGA |
| | GTTCAGAGTTTATTCTAGTGCGAATAATTGCACTTTTGAATA |
| | TGTCTCTCAGCCTTTTCTTATGGACCTTGAAGGAAAACAGGG |
| | TAATTTCAAAAATCTTAGGGAATTTGTGTTTAAGAATATTGA |
| | TGGTTATTTTAAAATATATTCTAAGCACACGCCTATTAATTT |
| | AGTGCGTGATCTCCCTCAGGGTTTTTCGGCTTTAGAACCATT |
| | GGTAGATTTGCCAATAGGTATTAACATCACTAGGTTTCAAAC |
| | TTTACTTGCTTTACATAGAAGTTATTTGACTCCTGGTGATTCT |
| | TCTTCAGGTTGGACAGCTGGTGCTGCAGCTTATTATGTGGGT |
| | TATCTTCAACCTAGGACTTTTCTATTAAAATATAATGAAAAT |
| | GGAACCATTACAGATGCTGTAGACTGTGCACTTGACCCTCTC |
| | TCAGAAACAAAGTGTACGTTGAAATCCTTCACTGTAGAAAA |
| | AGGAATCTATCAAACTTCTAACTTTAGAGTCCAACCAACAGA |
| | ATCTATTGTTAGATTTCCTAATATTACAAACTTGTGCCCTTTT |
| | GGTGAAGTTTTTAACGCCACCAGATTTGCATCTGTTTATGCT |
| | TGGAACAGGAAGAGAATCAGCAACTGTGTTGCTGATTATTCT |
| | GTCCTATATAATTCCGCATCATTTTCCACTTTTAAGTGTTATG |
| | GAGTGTCTCCTACTAAATTAAATGATCTCTGCTTTACTAATG |
| | TCTATGCAGATTCATTTGTAATTAGAGGTGATGAAGTCAGAC |
| | AAATCGCTCCAGGGCAAACTGGAAAGATTGCTGATTATAAT |
| | TATAAATTACCAGATGATTTTACAGGCTGCGTTATAGCTTGG |
| | AATTCTAACAATCTTGATTCTAAGGTTGGTGGTAATTATAAT |
| | TACCTGTATAGATTGTTTAGGAAGTCTAATCTCAAACCTTTT |
| | GAGAGAGATATTTCAACTGAAATCTATCAGGCCGGTAGCAC |
| | ACCTTGTAATGGTGTTGAAGGTTTTAATTGTTACTTTCCTTTA |
| | CAATCATATGGTTTCCAACCCACTAATGGTGTTGGTTACCAA |
| | CCATACAGAGTAGTAGTACTTTCTTTTGAACTTCTACATGCA |
| | CCAGCAACTGTTTGTGGACCTAAAAAGTCTACTAATTTGGTT |
| | AAAAACAAATGTGTCAATTTCAACTTCAATGGTTTAACAGGC |
| | ACAGGTGTTCTTACTGAGTCTAACAAAAAGTTTCTGCCTTTC |
| | CAACAATTTGGCAGAGACATTGCTGACACTACTGATGCTGTC |
| | CGTGATCCACAGACACTTGAGATTCTTGACATTACACCATGT |
| | TCTTTTGGTGGTGTCAGTGTTATAACACCAGGAACAAATACT |
| | TCTAACCAGGTTGCTGTTCTTTATCAGGGTGTTAACTGCACA |
| | GAAGTCCCTGTTGCTATTCATGCAGATCAACTTACTCCTACT |
| | TGGCGTGTTTATTCTACAGGTTCTAATGTTTTTCAAACACGTG |
| | CAGGCTGTTTAATAGGGGCTGAACATGTCAACAACTCATATG |
| | AGTGTGACATACCCATTGGTGCAGGTATATGCGCTAGTTATC |
| | AGACTCAGACTAATTCTCCTCGGCGGGCACGTAGTGTAGCTA |
| | GTCAATCCATCATTGCCTACACTATGTCACTTGGTGCAGAAA |
| | ATTCAGTTGCTTACTCTAATAACTCTATTGCCATACCCACAA |
| | ATTTTACTATTAGTGTTACCACAGAAATTCTACCAGTGTCTA |
| | TGACCAAGACATCAGTAGATTGTACAATGTACATTTGTGGTG |
| | ATTCAACTGAATGCAGCAATCTTTTGTTGCAATATGGCAGTT |
| | TTTGTACACAATTAAACCGTGCTTTAACTGGAATAGCTGTTG |
| | AACAAGACAAAAACACCCAAGAAGTTTTTGCACAAGTCAAA |
| | CAAATTTACAAAACACCACCAATTAAAGATTTTGGTGGTTTT |
| | AATTTTTCACAAATATTACCAGATCCATCAAAACCAAGCAAG |
| | AGGTCATTTATTGAAGATCTACTTTTCAACAAAGTGACACTT |
| | GCAGATGCTGGCTTCATCAAACAATATGGTGATTGCCTTGGT |
| | GATATTGCTGCTAGAGACCTCATTTGTGCACAAAAGTTTAAC |
| | GGCCTTACTGTTTTGCCACCTTTGCTCACAGATGAAATGATT |
| | GCTCAATACACTTCTGCACTGTTAGCGGGTACAATCACTTCT |
| | GGTTGGACCTTTGGTGCAGGTGCTGCATTACAAATACCATTT |
| | GCTATGCAAATGGCTTATAGGTTTAATGGTATTGGAGTTACA |
| | CAGAATGTTCTCTATGAGAACCAAAAATTGATTGCCAACCA |
| | ATTTAATAGTGCTATTGGCAAAATTCAAGACTCACTTTCTTC |
| | CACAGCAAGTGCACTTGGAAAACTTCAAGATGTGGTCAACC |
| | AAAATGCACAAGCTTTAAACACGCTTGTTAAACAACTTAGCT |
| | CCAATTTTGGTGCAATTTCAAGTGTTTTAAATGATATCCTTTC |
| | ACGTCTTGACAAAGTTGAGGCTGAAGTGCAAATTGATAGGT |
| | TGATCACAGGCAGACTTCAAAGTTTGCAGACATATGTGACTC |
| | AACAATTAATTAGAGCTGCAGAAATCAGAGCTTCTGCTAATC |
| | TTGCTGCTACTAAAATGTCAGAGTGTGTACTTGGACAATCAA |
| | AAAGAGTTGATTTTTGTGGAAAGGGCTATCATCTTATGTCCT |
| | TCCCTCAGTCAGCACCTCATGGTGTAGTCTTCTTGCATGTGA |
| | CTTATGTCCCTGCACAAGAAAAGAACTTCACAACTGCTCCTG |
| | CCATTTGTCATGATGGAAAAGCACACTTTCCTCGTGAAGGTG |
| | TCTTTGTTTCAAATGGCACACACTGGTTTGTAACACAAAGGA |
| | ATTTTTATGAACCACAAATCATTACTACAGACAACACATTTG |
| | TGTCTGGTAACTGTGATGTTGTAATAGGAATTGTCAACAACA |
| | CAGTTTATGATCCTTTGCAACCTGAATTAGACTCATTCAAGG |
| | AGGAGTTAGATAAATATTTTAAGAATCATACATCACCAGAT |
| | GTTGATTTAGGTGACATCTCTGGCATTAATGCTTCAGTTGTA |
| | AACATTCAAAAAGAAATTGACCGCCTCAATGAGGTTGCCAA |
| | GAATTTAAATGAATCTCTCATCGATCTCCAAGAACTTGGAAA |
| | GTATGAGCAGTATATAAAATGGCCATGGTACATTTGGCTAG |
| | GTTTTATAGCTGGCTTGATTGCCATAGTAATGGTGACAATTA |
| | TGCTTTGCTGTATGACCAGTTGCTGTAGTTGTCTCAAGGGCT |
| | GTTGTTCTTGTGGATCCTGCTGCAAATTTGATGAAGACGACT |
| | CTGAGCCAGTGCTCAAAGGAGTCAAATTACATTACACATAA |
| SEQ ID NO: 4 | MFVFLVLLPLVSSQCVNLTTRTQLPPAYTNSFTRGVYYPDKVFR |
| SARS-CoV-2 | SSVLHSTQDLFLPFFSNVTWFHAIHVSGTNGTKRFDNPVLPEND |
| D614G S Protein | GVYFASTEKSNIIRGWIFGTTLDSKTQSLLIVNNATNVVIKVCEF |
| (amino acid | QFCNDPFLGVYYHKNNKSWMESEFRVYSSANNCTFEYVSQPFL |
| sequence) | MDLEGKQGNFKNLREFVFKNIDGYFKIYSKHTPINLVRDLPQGF |
| | SALEPLVDLPIGINITRFQTLLALHRSYLTPGDSSSGWTAGAAAY |
| | YVGYLQPRTFLLKYNENGTITDAVDCALDPLSETKCTLKSFTVE |
| | KGIYQTSNFRVQPTESIVRFPNITNLCPFGEVENATRFASVYAW |
| | NRKRISNCVADYSVLYNSASFSTFKCYGVSPTKLNDLCFTNVY |
| | ADSFVIRGDEVRQIAPGQTGKIADYNYKLPDDFTGCVIAWNSN |
| | NLDSKVGGNYNYLYRLFRKSNLKPFERDISTEIYQAGSTPCNGV |
| | EGFNCYFPLQSYGFQPTNGVGYQPYRVVVLSFELLHAPATVCG |
| | PKKSTNLVKNKCVNFNFNGLTGTGVLTESNKKFLPFQQFGRDI |
| | ADTTDAVRDPQTLEILDITPCSFGGVSVITPGTNTSNQVAVLYQ |
| | GVNCTEVPVAIHADQLTPTWRVYSTGSNVFQTRAGCLIGAEHV |
| | NNSYECDIPIGAGICASYQTQTNSPRRARSVASQSIIAYTMSLGA |
| | ENSVAYSNNSIAIPTNFTISVTTEILPVSMTKTSVDCTMYICGDST |
| | ECSNLLLQYGSFCTQLNRALTGIAVEQDKNTQEVFAQVKQIYK |
| | TPPIKDFGGFNFSQILPDPSKPSKRSFIEDLLFNKVTLADAGFIKQ |
| | YGDCLGDIAARDLICAQKFNGLTVLPPLLTDEMIAQYTSALLAG |
| | TITSGWTFGAGAALQIPFAMQMAYRENGIGVTQNVLYENQKLI |
| | ANQFNSAIGKIQDSLSSTASALGKLQDVVNQNAQALNTLVKQL |
| | SSNFGAISSVLNDILSRLDKVEAEVQIDRLITGRLQSLQTYVTQQ |
| | LIRAAEIRASANLAATKMSECVLGQSKRVDFCGKGYHLMSFPQ |
| | SAPHGVVFLHVTYVPAQEKNFTTAPAICHDGKAHFPREGVFVS |
| | NGTHWFVTQRNFYEPQIITTDNTFVSGNCDVVIGIVNNTVYDPL |
| | QPELDSFKEELDKYFKNHTSPDVDLGDISGINASVVNIQKEIDRL |
| | NEVAKNLNESLIDLQELGKYEQYIKWPWYIWLGFIAGLIAIVMV |
| | TIMLCCMTSCCSCLKGCCSCGSCCKFDEDDSEPVLKGVKLHYT |
| SEQ ID NO: 5 | CGGGTCCAACCCACCGAAAGCATTGTGCGGTTCCCAAATATC |
| SARS-CoV-2 | ACCAATCTGTGTCCCTTTGGCGAAGTGTTCAATGCTACAAGG |
| RBD S Protein | TTTGCTTCTGTGTACGCATGGAATAGGAAACGCATCTCCAAT |
| (nucleic acid | TGTGTCGCTGATTACTCCGTGCTGTACAATTCCGCCTCTTTCT |
| sequence) | CAACCTTCAAGTGTTATGGCGTTTCACCTACCAAACTTAACG |
| | ACCTGTGCTTCACTAATGTGTATGCCGACTCTTTTGTGATAC |
| | GAGGCGATGAAGTGAGACAGATTGCACCAGGGCAGACCGGC |
| | AAAATTGCCGACTACAACTACAAGCTTCCAGATGACTTTACC |
| | GGATGTGTTATTGCATGGAACTCAAACAATCTGGATTCCAAG |
| | GTGGGTGGCAACTATAACTACCTGTATAGACTGTTCAGGAA |
| | ATCCAACCTGAAACCATTCGAGCGAGATATAAGCACAGAAA |
| | TCTACCAGGCTGGAAGTACGCCCTGCAACGGCGTGGAAGGG |
| | TTCAACTGCTACTTCCCATTGCAGAGTTACGGATTCCAGCCT |
| | ACAAACGGGGTGGGTTACCAACCCTATCGTGTCGTAGTCCTG |
| | AGTTTTGAGCTCCTCCATGCCCCAGCCACAGTCTGTGGCCCC |
| | AAGAAAAGCACCAATCTGGTGAAGAACAAATGCGTGAACTT |
| | TTAG |
| SEQ ID NO: 6 | RVQPTESIVRFPNITNLCPFGEVENATRFASVYAWNRKRISNCV |
| SARS-CoV-2 | ADYSVLYNSASFSTFKCYGVSPTKLNDLCFTNVYADSFVIRGDE |
| RBD S Protein | VRQIAPGQTGKIADYNYKLPDDFTGCVIAWNSNNLDSKVGGN |
| (amino acid | YNYLYRLFRKSNLKPFERDISTEIYQAGSTPCNGVEGENCYFPL |
| sequence) | QSYGFQPTNGVGYQPYRVVVLSFELLHAPATVCGPKKSTNLVK |
| | NKCVNF |
| SEQ ID NO: 7 | ATGGCAGATTCCAACGGTACTATTACCGTTGAGGAACTTAAA |
| SARS-CoV-2 M | AAGCTCCTTGAACAATGGAACCTAGTAATAGGTTTCCTATTC |
| Protein (nucleic | CTTACATGGATTTGTCTTCTACAATTTGCCTATGCCAACAGG |
| acid sequence) | AATAGGTTTTTGTATATAATTAAGTTAATTTTCCTCTGGCTGT |
| | TATGGCCAGTAACTTTAGCTTGTTTTGTGCTTGCTGCTGTTTA |
| | CAGAATAAATTGGATCACCGGTGGAATTGCTATCGCAATGG |
| | CTTGTCTTGTAGGCTTGATGTGGCTCAGCTACTTCATTGCTTC |
| | TTTCAGACTGTTTGCGCGTACGCGTTCCATGTGGTCATTCAA |
| | TCCAGAAACTAACATTCTTCTCAACGTGCCACTCCATGGCAC |
| | TATTCTGACCAGACCGCTTCTAGAAAGTGAACTCGTAATCGG |
| | AGCTGTGATCCTTCGTGGACATCTTCGTATTGCTGGACACCA |
| | TCTAGGACGCTGTGACATCAAGGACCTGCCTAAAGAAATCA |
| | CTGTTGCTACATCACGAACGCTTTCTTATTACAAATTGGGAG |
| | CTTCGCAGCGTGTAGCAGGTGACTCAGGTTTTGCTGCATACA |
| | GTCGCTACAGGATTGGCAACTATAAATTAAACACAGACCAT |
| | TCCAGTAGCAGTGACAATATTGCTTTGCTTGTACAGTAA |
| SEQ ID NO: 8 | MADSNGTITVEELKKLLEQWNLVIGFLFLTWICLLQFAYANRN |
| SARS-CoV-2 M | RFLYIIKLIFLWLLWPVTLACFVLAAVYRINWITGGIAIAMACLV |
| Protein (amino | GLMWLSYFIASFRLFARTRSMWSFNPETNILLNVPLHGTILTRPL |
| acid sequence) | LESELVIGAVILRGHLRIAGHHLGRCDIKDLPKEITVATSRTLSY |
| | YKLGASQRVAGDSGFAAYSRYRIGNYKLNTDHSSSSDNIALLV |
| | Q |
| SEQ ID NO: 9 | ATGGCAGATTCCAACGGTACTATTACCGTTGAAGAGCTTAAA |
| WA9-UW6 | AAGCTCCTTGAACAATGGAACCTAGTAATAGGTTTCCTATTC |
| Variant M | CTTACATGGATTTGTCTTCTACAATTTGCCTATGCCAACAGG |
| Protein (nucleic | AATAGGTTTTTGTATATAATTAAGTTAATTTTCCTCTGGCTGT |
| acid sequence) | TATGGCCAGTAACTTTAGCTTGTTTTGTGCTTGCTGCTATTTA |
| | CAGAATAAATTGGATCACCGGTGGAATTGCTATCGCAATGG |
| | CTTGTCTTGTAGGCTTGATGTGGCTCAGCTACTTCATTGCTTC |
| | TTTCAGACTGTTTGCGCGTACGCGTTCCATGTGGTCATTCAA |
| | TCCAGAAACTAACATTCTTCTCAACGTGCCACTCCATGGCAC |
| | TATTCTGACCAGACCGCTTCTAGAAAGTGAACTCGTAATCGG |
| | AGCTGTGATCCTTCGTGGACATCTTCGTATTGCTGGACACCA |
| | TCTAGGACGCTGTGACATCAAGGACCTGCCTAAAGAAATCA |
| | CTGTTGCTACATCACGAACGCTTTCTTATTACAAATTGGGAG |
| | CTTCGCAGCGTGTAGCAGGTGACTCAGGTTTTGCTGCATACA |
| | GTCGCTACAGGATTGGCAACTATAAATTAAACACAGACCAT |
| | TCCAGTAGCAGTGACAATATTGCTTTGCTTGTACAGTAA |
| SEQ ID NO: 10 | MADSNGTITVEELKKLLEQWNLVIGFLFLTWICLLQFAYANRN |
| WA9-UW6 | RFLYIIKLIFLWLLWPVTLACFVLAAIYRINWITGGIAIAMACLV |
| Variant M | GLMWLSYFIASFRLFARTRSMWSFNPETNILLNVPLHGTILTRPL |
| Protein (amino | LESELVIGAVILRGHLRIAGHHLGRCDIKDLPKEITVATSRTLSY |
| acid sequence) | YKLGASQRVAGDSGFAAYSRYRIGNYKLNTDHSSSSDNIALLV |
| | Q |
| SEQ ID NO: 11 | ATGGCAGATTCCAACGGTACTATTACCGTTGAAGAGCTTAAA |
| NIHE Variant M | AAGCTCCTTGAACAATGGAACCTAGTAATAGGTTTCCTATTC |
| Protein (nucleic | CTTACATGGATTTGTCTTCTACAATTTGCCTATGCCAACAGG |
| acid sequence) | AATAGGTTTTTGTATATAATTAAGTTAATTTTCCTCTGGCTGG |
| | TATGGCCAGTAACTTTAGCTTGTTTTGTGCTTGCTGCCGTTTA |
| | CAGAATAAATTGGATCACCGGTGGAATTGCTATCGCAATGG |
| | CTTGTCTTGTACGCTTGATGTGGCTCAGCTACTTCATTGCTTC |
| | TTTCAGACTGTTTGCGCGTACGCGTTCCATGTGGTCATTCAA |
| | TCCAGAAACTAACATTCTTCTCAACGTGCCACTCCATGGCAC |
| | TATTCTGACCAGACCGCTTCTAGAAAGTGAACTCGTAATCGG |
| | AGCTGTGATCCTTCGTGGACATCTTCGTATTGCTGGACACCA |
| | TCTAGGACGCTGTGACATCAAGGACCTGCCTAAAGAAATCA |
| | CTGTTGCTACATCACGAACGCTTTCTTATTACAAATTGGGAG |
| | CTTCGCAGCGTGTAGCAGGTGACTCAGGTTTTGCTGCATACA |
| | GTCGCTACAGGATTGGCAACTATAAATTAAACACAGACCAT |
| | TCCAGTAGCAGTGACAATATTGCTTTGCTTGTACAGTAA |
| SEQ ID NO: 12 | MADSNGTITVEELKKLLEQWNLVIGFLFLTWICLLQFAYANRN |
| NIHE Variant M | RFLYIIKLIFLWLVWPVTLACFVLAAVYRINWITGGIAIAMACL |
| Protein (amino | VRLMWLSYFIASFRLFARTRSMWSFNPETNILLNVPLHGTILTRP |
| acid sequence) | LLESELVIGAVILRGHLRIAGHHLGRCDIKDLPKEITVATSRTLS |
| | YYKLGASQRVAGDSGFAAYSRYRIGNYKLNTDHSSSSDNIALL |
| | VQ |
| SEQ ID NO: 13 | ATGTCAGATTCCAACGGTACTATTACCGTTGAAGAGCTTAAA |
| Wuhan_IME_BJO7_ | AAGCTCCTTGAACAATGGAACCTAGTAATAGGTTTCCTATTC |
| Variant M | CTTACATGGATTTGTCTTCTACAATTTGCCTATGCCAACAGG |
| Protein (nucleic | AATAGGTTTTTGTATATAATTAAGTTAATTTTCCTCTGGCTGT |
| acid sequence) | TATGGCCAGTAACTTTAGCTTGTTTTGTGCTTGCTGCTGTTTA |
| | CAGAATAAATTGGATCACCGGTGGAATTGCTATCGCAATGG |
| | CTTGTCTTGTAGGCTTGATGTGGCTCAGCTACTTCATTGCTTC |
| | TTTCAGACTGTTTGCGCGTACGCGTTCCATGTGGTCATTCAA |
| | TCCAGAAACTAACATTCTTCTCAACGTGCCACTCCATGGCAC |
| | TATTCTGACCAGACCGCTTCTAGAAAGTGAACTCGTAATCGG |
| | AGCTGTGATCCTTCGTGGACATCTTCGTATTGCTGGACACCA |
| | TCTAGGACGCTGTGACATCAAGGACCTGCCTAAAGAAATCA |
| | CTGTTGCTACATCACGAACGCTTTCTTATTACAAATTGGGAG |
| | CTTCGCAGCGTGTAGCAGGTGACTCAGGTTTTGCTGCATACA |
| | GTCGCTACAGGATTGGCAACTATAAATTAAACACAGACCAT |
| | TCCAGTAGCAGTGACAATATTGCTTTGCTTGTACAGTAA |
| SEQ ID NO: 14 | MSDSNGTITVEELKKLLEQWNLVIGFLFLTWICLLQFAYANRNR |
| Wuhan_IME_BJO7_ | FLYIIKLIFLWLLWPVTLACFVLAAVYRINWITGGIAIAMACLVG |
| Variant M | LMWLSYFIASFRLFARTRSMWSFNPETNILLNVPLHGTILTRPLL |
| Protein (amino | ESELVIGAVILRGHLRIAGHHLGRCDIKDLPKEITVATSRTLSYY |
| acid sequence) | KLGASQRVAGDSGFAAYSRYRIGNYKLNTDHSSSSDNIALLVQ |
| SEQ ID NO: 15 | ATGGCAGATTCCAACGGTACTATTACCGTTGAAGAGCTTAAA |
| WA-UW-1753 | AAGCTCCTTGAACAATGGAACCTAGTAATAGGTTTCCTATTC |
| Variant M | CTTACATGGATTTGTCTTCTACAATTTGCCTATGCCAACAGG |
| Protein (nucleic | AATAGGTTTTTGTATATAATTAAGTTAATTTTCCTCTGGCTGT |
| acid sequence) | TATGGCCAGTAACTTTAGCTTGTTTTGTGCTTGCTGCTGTTTA |
| | CAGAATAAATTGGATCACCGGTGGAATTGCTATCGCAATGTC |
| | TTGTCTTGTAGGCTTGATGTGGCTCAGCTACTTCATTGCTTCT |
| | TTCAGACTGTTTGCGCGTACGCGTTCCATGTGGTCATTCAAT |
| | CCAGAAACTAACATTCTTCTCAACGTGCCACTCCATGGCACT |
| | ATTCTGACCAGACCGCTTCTAGAAAGTGAACTCGTAATCGGA |
| | GCTGTGATCCTTCGTGGACATCTTCGTATTGCTGGACACCAT |
| | CTAGGACGCTGTGACATCAAGGACCTGCCTAAAGAAATCAC |
| | TGTTGCTACATCACGAACGCTTTCTTATTACAAATTGGGAGC |
| | TTCGCAGCGTGTAGCAGGTGACTCAGGTTTTGCTGCATACAG |
| | TCGCTACAGGATTGGCAACTATAAATTAAACACAGACCATTC |
| | CAGTAGCAGTGACAATATTGCTTTGCTTGTACAGTAA |
| SEQ ID NO: 16 | MADSNGTITVEELKKLLEQWNLVIGFLFLTWICLLQFAYANRN |
| WA-UW-1753 | RFLYIIKLIFLWLLWPVTLACFVLAAVYRINWITGGIAIAMSCLV |
| Variant M | GLMWLSYFIASFRLFARTRSMWSFNPETNILLNVPLHGTILTRPL |
| Protein (amino | LESELVIGAVILRGHLRIAGHHLGRCDIKDLPKEITVATSRTLSY |
| acid sequence) | YKLGASQRVAGDSGFAAYSRYRIGNYKLNTDHSSSSDNIALLV |
| | Q |
| SEQ ID NO: 17 | ATGGCAGATTCCAACGGTACTATTACCGTTGAAGAGCTTAAA |
| WA UW-1755 | AAGCTCCTTGAACAATGGAACCTAGTAATAGGTTTCCTATTC |
| Variant M | CTTACATGGATTTGTCTTCTACAATTTGCCTATGCCAACAGG |
| Protein (nucleic | AATAGGTTTTTGTATATAATTAAGTTAATTTTCCTCTGGCTGT |
| acid sequence) | TATGGCCAGTAACTTTAGCTTGTTTTGTGCTTGCTGCTGTTTA |
| | CAGAATAAATTGGATCACCGGTGGAATTGCTATCGCAATGG |
| | CTTGTCTTGTAGGCTTGATGTGGCTCAGCTACTTCATTGCTTC |
| | TTTCAGACTGTTTGCGCGTACGCGTTCCATGTGGTCATTCAA |
| | TCCAGAAACTAACATTCTTCTCAACGTGCCACTCCATGGCAC |
| | TATTCTGACCAGACCGCTTCTAGAAAGTGAACTCGTAATCGG |
| | AGCTGTGATCCTTCGTGGACATCTTCGTATTGCTGGACACCA |
| | TCTAGGACGCTGTGACATCAAGGACCTGCCTAAAGAAATCA |
| | CTGTTGCTACATCACGAATGCTTTCTTATTACAAATTGGGAG |
| | CTTCGCAGCGTGTAGCAGGTGACTCAGGTTTTGCTGCATACA |
| | GTCGCTACAGGATTGGCAACTATAAATTAAACACAGACCAT |
| | TCCAGTAGCAGTGACAATATTGCTTTGCTTGTACAGTAA |
| SEQ ID NO: 18 | MADSNGTITVEELKKLLEQWNLVIGFLFLTWICLLQFAYANRN |
| WA UW-1755 | RFLYIIKLIFLWLLWPVTLACFVLAAVYRINWITGGIAIAMACLV |
| Variant M | GLMWLSYFIASFRLFARTRSMWSFNPETNILLNVPLHGTILTRPL |
| Protein (amino | LESELVIGAVILRGHLRIAGHHLGRCDIKDLPKEITVATSRMLSY |
| acid sequence) | YKLGASQRVAGDSGFAAYSRYRIGNYKLNTDHSSSSDNIALLV |
| | Q |
| SEQ ID NO: 19 | ATGGCAGGTTCCAACGGTACTATTACCGTTGAAGAGCTTAAA |
| USA_SC_3572 | AAGCTCCTTGAACAATGGAACCTAGTAATAGGTTTCCTATTC |
| Variant M | CTTACATGGATTTGTCTTCTACAATTTGCCTATGCCAACAGG |
| Protein (nucleic | AATAGGTTTTTGTATATAATTAAGTTAATTTTCCTCTGGCTGT |
| acid sequence) | TATGGCCAGTAACTTTAGCTTGTTTTGTGCTTGCTGCTGTTTA |
| | CAGAATAAATTGGATCACCGGTGGAATTGCTATCGCAATGG |
| | CTTGTCTTGTAGGCTTGATGTGGCTCAGCTACTTCATTGCTTC |
| | TTTCAGACTGTTTGCGCGTACGCGTTCCATGTGGTCATTCAA |
| | TCCAGAAACTAACATTCTTCTCAACGTGCCACTCCATGGCAC |
| | TATTCTGACCAGACCGCTTCTAGAAAGTGAACTCGTAATCGG |
| | AGCTGTGATCCTTCGTGGACATCTTCGTATTGCTGGACACCA |
| | TCTAGGACGCTGTGACATCAAGGACCTGCCTAAAGAAATCA |
| | CTGTTGCTACATCACGAACGCTTTCTTATTACAAATTGGGAG |
| | CTTCGCAGCGTGTAGCAGGTGACTCAGGTTTTGCTGCATACA |
| | GTCGCTACAGGATTGGCAACTATAAATTAAACACAGACCAT |
| | TCCAGTAGCAGTGACAATATTGCTTTGCTTGTACAGTAA |
| SEQ ID NO: 20 | MAGSNGTITVEELKKLLEQWNLVIGFLFLTWICLLQFAYANRN |
| USA_SC_3572 | RFLYIIKLIFLWLLWPVTLACFVLAAVYRINWITGGIAIAMACLV |
| Variant M | GLMWLSYFIASFRLFARTRSMWSFNPETNILLNVPLHGTILTRPL |
| Protein (amino | LESELVIGAVILRGHLRIAGHHLGRCDIKDLPKEITVATSRTLSY |
| acid sequence) | YKLGASQRVAGDSGFAAYSRYRIGNYKLNTDHSSSSDNIALLV |
| | Q |
| SEQ ID NO: 21 | ATGTACTCATTCGTTTCGGAAGAGACAGGTACGTTAATAGTT |
| SARS-CoV-2 E | AATAGCGTACTTCTTTTTCTTGCTTTCGTGGTATTCTTGCTAG |
| Protein (nucleic | TTACACTAGCCATCCTTACTGCGCTTCGATTGTGTGCGTACT |
| acid sequence) | GCTGCAATATTGTTAACGTGAGTCTTGTAAAACCTTCTTTTTA |
| | CGTTTACTCTCGTGTTAAAAATCTGAATTCTTCTAGAGTTCCT |
| | GATCTTCTGGTCTAA |
| SEQ ID NO: 22 | MYSFVSEETGTLIVNSVLLFLAFVVFLLVTLAILTALRLCAYCC |
| SARS-CoV-2 E | NIVNVSLVKPSFYVYSRVKNLNSSRVPDLLV |
| Protein (amino | |
| acid sequence) | |
| SEQ ID NO: 23 | ATGTACTCATTCGTTTCGGAAGAGACAGGTACGTTAATAGTT |
| USA_WA-UW- | AATAGCGTACTTCTTTTTCTTGCTTTCGTGGTATTCTTGCTAG |
| 1588 Variant E | TTACACTAGCCATCCTTACTGCGCTTCGATTGTGTGCGTACT |
| Protein (nucleic | GCTGCAATATTGTTAACGTGAGTCTTGTAAAACCTTCTTTTTA |
| acid sequence) | CGTTTACTCTCGTGTTAAAAATCTGAATTCTTCTAGAGTTCTT |
| | GATCTTCTGGTCTAA |
| SEQ ID NO: 24 | MYSFVSEETGTLIVNSVLLFLAFVVFLLVTLAILTALRLCAYCC |
| USA_WA-UW- | NIVNVSLVKPSFYVYSRVKNLNSSRVLDLLV |
| 1588 Variant E | |
| Protein (amino | |
| acid sequence) | |
| SEQ ID NO: 25 | ATGTACTCATTCGTTTCGGAAGAGACAGGTACGTTAATAGTT |
| SNU01 Variant | AATAGCGTACTTCTTTTTCTTGCTTTCGTGGTATTCTTGCTAG |
| E Protein | TTACACTAGCCATCCTTACTGCGCATCGATTGTGTGCGTACT |
| (nucleic acid | GCTGCAATATTGTTAACGTGAGTCTTGTAAAACCTTCTTTTTA |
| sequence) | CGTTTACTCTCGTGTTAAAAATCTGAATTCTTCTAGAGTTCCT |
| | GATCTTCTGGTCTAA |
| SEQ ID NO: 26 | MYSFVSEETGTLIVNSVLLFLAFVVFLLVTLAILTAHRLCAYCC |
| SNU01 Variant | NIVNVSLVKPSFYVYSRVKNLNSSRVPDLLV |
| E Protein (amino | |
| acid sequence) | |
| SEQ ID NO: 27 | ATGTCTGATAATGGACCCCAAAATCAGCGAAATGCACCCCG |
| SARS-CoV-2 N | CATTACGTTTGGTGGACCCTCAGATTCAACTGGCAGTAACCA |
| Protein (nucleic | GAATGGAGAACGCAGTGGGGCGCGATCAAAACAACGTCGGC |
| acid sequence) | CCCAAGGTTTACCCAATAATACTGCGTCTTGGTTCACCGCTC |
| | TCACTCAACATGGCAAGGAAGACCTTAAATTCCCTCGAGGA |
| | CAAGGCGTTCCAATTAACACCAATAGCAGTCCAGATGACCA |
| | AATTGGCTACTACCGAAGAGCTACCAGACGAATTCGTGGTG |
| | GTGACGGTAAAATGAAAGATCTCAGTCCAAGATGGTATTTCT |
| | ACTACCTAGGAACTGGGCCAGAAGCTGGACTTCCCTATGGT |
| | GCTAACAAAGACGGCATCATATGGGTTGCAACTGAGGGAGC |
| | CTTGAATACACCAAAAGATCACATTGGCACCCGCAATCCTGC |
| | TAACAATGCTGCAATCGTGCTACAACTTCCTCAAGGAACAAC |
| | ATTGCCAAAAGGCTTCTACGCAGAAGGGAGCAGAGGCGGCA |
| | GTCAAGCCTCTTCTCGTTCCTCATCACGTAGTCGCAACAGTT |
| | CAAGAAATTCAACTCCAGGCAGCAGTAGGGGAACTTCTCCT |
| | GCTAGAATGGCTGGCAATGGCGGTGATGCTGCTCTTGCTTTG |
| | CTGCTGCTTGACAGATTGAACCAGCTTGAGAGCAAAATGTCT |
| | GGTAAAGGCCAACAACAACAAGGCCAAACTGTCACTAAGAA |
| | ATCTGCTGCTGAGGCTTCTAAGAAGCCTCGGCAAAAACGTA |
| | CTGCCACTAAAGCATACAATGTAACACAAGCTTTCGGCAGA |
| | CGTGGTCCAGAACAAACCCAAGGAAATTTTGGGGACCAGGA |
| | ACTAATCAGACAAGGAACTGATTACAAACATTGGCCGCAAA |
| | TTGCACAATTTGCCCCCAGCGCTTCAGCGTTCTTCGGAATGT |
| | CGCGCATTGGCATGGAAGTCACACCTTCGGGAACGTGGTTG |
| | ACCTACACAGGTGCCATCAAATTGGATGACAAAGATCCAAA |
| | TTTCAAAGATCAAGTCATTTTGCTGAATAAGCATATTGACGC |
| | ATACAAAACATTCCCACCAACAGAGCCTAAAAAGGACAAAA |
| | AGAAGAAGGCTGATGAAACTCAAGCCTTACCGCAGAGACAG |
| | AAGAAACAGCAAACTGTGACTCTTCTTCCTGCTGCAGATTTG |
| | GATGATTTCTCCAAACAATTGCAACAATCCATGAGCAGTGCT |
| | GACTCAACTCAGGCCTAA |
| SEQ ID NO: 28 | MSDNGPQNQRNAPRITFGGPSDSTGSNQNGERSGARSKQRRPQ |
| SARS-CoV-2 N | GLPNNTASWFTALTQHGKEDLKFPRGQGVPINTNSSPDDQIGY |
| Protein (amino | YRRATRRIRGGDGKMKDLSPRWYFYYLGTGPEAGLPYGANKD |
| acid sequence) | GIIWVATEGALNTPKDHIGTRNPANNAAIVLQLPQGTTLPKGFY |
| | AEGSRGGSQASSRSSSRSRNSSRNSTPGSSRGTSPARMAGNGGD |
| | AALALLLLDRLNQLESKMSGKGQQQQGQTVTKKSAAEASKKP |
| | RQKRTATKAYNVTQAFGRRGPEQTQGNFGDQELIRQGTDYKH |
| | WPQIAQFAPSASAFFGMSRIGMEVTPSGTWLTYTGAIKLDDKD |
| | PNFKDQVILLNKHIDAYKTFPPTEPKKDKKKKADETQALPQRQ |
| | KKQQTVTLLPAADLDDFSKQLQQSMSSADSTQA |
| SEQ ID NO: 29 | CTAGCTGGCCAGACATGATAAGATACATTGATGAGTTTGGA |
| SV40 p(A) tail | CAAACCACAACTAGAATGCAGTGAAAAAAATGCTTTATTTG |
| (nucleic acid | TGAAATTTGTGATGCTATTGCTTTATTTGTAACCATTATAAGC |
| sequence) | TGCAATAAACAAGTTAACAACAACAATTGCATTCATTTTATG |
| | TTTCAGGTTCAGGGGGAGGTGTGGGAGGTTTTTTAAAGCAA |
| | GTAAAACCTCTACAAATGTGGTATGG |
| SEQ ID NO: 30 | CTGTGCCTTCTAGTTGCCAGCCATCTGTTGTTTGCCCCTCCCC |
| bGH p(A) tail | CGTGCCTTCCTTGACCCTGGAAGGTGCCACTCCCACTGTCCT |
| (nucleic acid | TTCCTAATAAAATGAGGAAATTGCATCGCATTGTCTGAGTAG |
| sequence) | GTGTCATTCTATTCTGGGGGGTGGGGTGGGGCAGGACAGCA |
| | AGGGGGAGGATTGGGAAGACAATAGCAGGCATGCTGGGGA |
| | TGCGGTGGGCTCTATGG |
| SEQ ID NO: 31 | TCAATATTGGCCATTAGCCATATTATTCATTGGTTATATAGC |
| Human CMV | ATAAATCAATATTGGCTATTGGCCATTGCATACGTTGTATCT |
| promoter | ATATCATAATATGTACATTTATATTGGCTCATGTCCAATATG |
| (nucleic acid | ACCGCCATGTTGGCATTGATTATTGACTAGTTATTAATAGTA |
| sequence) | ATCAATTACGGGGTCATTAGTTCATAGCCCATATATGGAGTT |
| | CCGCGTTACATAACTTACGGTAAATGGCCCGCCTGGCTGACC |
| | GCCCAACGACCCCCGCCCATTGACGTCAATAATGACGTATGT |
| | TCCCATAGTAACGCCAATAGGGACTTTCCATTGACGTCAATG |
| | GGTGGAGTATTTACGGTAAACTGCCCACTTGGCAGTACATCA |
| | AGTGTATCATATGCCAAGTCCGCCCCCTATTGACGTCAATGA |
| | CGGTAAATGGCCCGCCTGGCATTATGCCCAGTACATGACCTT |
| | ACGGGACTTTCCTACTTGGCAGTACATCTACGTATTAGTCAT |
| | CGCTATTACCATGGTGATGCGGTTTTGGCAGTACACCAATGG |
| | GCGTGGATAGCGGTTTGACTCACGGGGATTTCCAAGTCTCCA |
| | CCCCATTGACGTCAATGGGAGTTTGTTTTGGCACCAAAATCA |
| | ACGGGACTTTCCAAAATGTCGTAATAACCCCGCCCCGTTGAC |
| | GCAAATGGGCGGTAGGCGTGTACGGTGGGAGGTCTATATAA |
| | GCAGAGCTCGTTTAGTGAACCGTCAGATC |
| SEQ ID NO: 32 | GCGCAGCACCATGGCCTGAAATAACCTCTGAAAGAGGAACT |
| SV40 promoter | TGGTTAGGTACCTTCTGAGGCGGAAAGAACCAGCTGTGGAA |
| (nucleic acid | TGTGTGTCAGTTAGGGTGTGGAAAGTCCCCAGGCTCCCCAGC |
| sequence) | AGGCAGAAGTATGCAAAGCATGCATCTCAATTAGTCAGCAA |
| | CCAGGTGTGGAAAGTCCCCAGGCTCCCCAGCAGGCAGAAGT |
| | ATGCAAAGCATGCATCTCAATTAGTCAGCAACCATAGTCCCG |
| | CCCCTAACTCCGCCCATCCCGCCCCTAACTCCGCCCAGTTCC |
| | GCCCATTCTCCGCCCCATGGCTGACTAATTTTTTTTATTTATG |
| | CAGAGGCCGAGGCCGCCTCGGCCTCTGAGCTATTCCAGAAG |
| | TAGTGAGGAGGCTTTTTTGGAGGCCTAGGCTTTTGCAAAAAG |
| | CTT |
| SEQ ID NO: 33 | CGTTACATAACTTACGGTAAATGGCCCGCCTGGCTGACCGCC |
| CAG promoter | CAACGACCCCCGCCCATTGACGTCAATAATGACGTATGTTCC |
| (nucleic acid | CATAGTAACGCCAATAGGGACTTTCCATTGACGTCAATGGGT |
| sequence) | GGAGTATTTACGGTAAACTGCCCACTTGGCAGTACATCAAGT |
| | GTATCATATGCCAAGTACGCCCCCTATTGACGTCAATGACGG |
| | TAAATGGCCCGCCTGGCATTATGCCCAGTACATGACCTTATG |
| | GGACTTTCCTACTTGGCAGTACATCTACGTATTAGTCATCGC |
| | TATTACCATGGTGATGCGGTTTTGGCAGTACATCAATGGGCG |
| | TGGATAGCGGTTTGACTCACGGGGATTTCCAAGTCTCCACCC |
| | CATTGACGTCAATGGGAGTTTGTTTTGGCACCAAAATCAACG |
| | GGACTTTCCAAAATGTCGTAACAACTCCGCCCCATTGACGCA |
| | AATGGGCGGTAGGCGTGTACGGTGGGAGGTCTATATAAGCA |
| | GAGCT |
| SEQ ID NO: 34 | PPFGAGRPGQFGDI |
| Chikungunya | |
| virus antigen | |
| (amino acid | |
| sequence) | |
| SEQ ID NO: 35 | TAECKDKNL |
| Chikungunya | |
| virus antigen | |
| (amino acid | |
| sequence) | |
| SEQ ID NO: 36 | VRYKCNCGG |
| Chikungunya | |
| virus antigen | |
| (amino acid | |
| sequence) | |
| SEQ ID NO: 37 | GGACCTGCAGGGCCTGAAATAACCTCTGAAAGAGGAACTTG |
| SV40 Enhancer | GTTAGGTACCTTCTGAGGCGGAAAGAACCAGCTGTGGAATG |
| (nucleic acid | TGTGTCAGTTAGGGTGTGGAAAGTCCCCAGGCTCCCCAGCA |
| sequence) | GGCAGAAGTATGCAAAGCATGCATCTCAATTAGTCAGCAAC |
| | CAGGTGTGGAAAGTCCCCAGGCTCCCCAGCAGGCAGAAGTA |
| | TGCAAAGCATGCATCTCAATTAGTCAGCAACCATAGTCCCAC |
| | TAGT |
| SEQ ID NO: 38 | GCTCCGGTGCCCGTCAGTGGGCAGAGCGCACATCGCCCACA |
| hEF1-HTLV | GTCCCCGAGAAGTTGGGGGGAGGGGTCGGCAATTGAACGGG |
| Promoter | TGCCTAGAGAAGGTGGCGCGGGGTAAACTGGGAAAGTGATG |
| (nucleic acid | TCGTGTACTGGCTCCGCCTTTTTCCCGAGGGTGGGGGAGAAC |
| sequence) | CGTATATAAGTGCAGTAGTCGCCGTGAACGTTCTTTTTCGCA |
| | ACGGGTTTGCCGCCAGAACACAGCTGAAGCTTCGAGGGGCT |
| | CGCATCTCTCCTTCACGCGCCCGCCGCCCTACCTGAGGCCGC |
| | CATCCACGCCGGTTGAGTCGCGTTCTGCCGCCTCCCGCCTGT |
| | GGTGCCTCCTGAACTGCGTCCGCCGTCTAGGTAAGTTTAAAG |
| | CTCAGGTCGAGACCGGGCCTTTGTCCGGCGCTCCCTTGGAGC |
| | CTACCTAGACTCAGCCGGCTCTCCACGCTTTGCCTGACCCTG |
| | CTTGCTCAACTCTACGTCTTTGTTTCGTTTTCTGTTCTGCGCA |
| | GTTACAGATCCAAGCTGTGACCGGCGCCTACCTGAGATCACC |
| | GGTCACCATGGAGATCAAGGTGCTGTTTGCCCTCATCTGTAT |
| | TGCTGTTGCTGAGGCA |
| SEQ ID NO: 39 | GTTAATCTCACCACCCGAACTCAACTCCCACCCGCATATACA |
| SARS-CoV-2 | AATTCCTTCACCAGAGGAGTGTACTATCCTGACAAAGTGTTT |
| D614G Spike | CGGTCAAGTGTCCTCCACTCTACTCAGGACCTCTTTCTGCCTT |
| Truncated S1 | TCTTTTCTAACGTTACATGGTTTCATGCAATCCATGTGTCTGG |
| (nucleic acid | GACAAACGGCACCAAACGCTTCGACAACCCTGTATTGCCATT |
| sequence) | CAATGATGGGGTGTACTTTGCCTCCACAGAGAAATCCAACAT |
| | CATTCGAGGATGGATTTTCGGGACTACTCTGGACTCAAAGAC |
| | ACAGAGCCTGCTGATCGTTAACAACGCCACAAACGTTGTCAT |
| | CAAAGTGTGCGAATTCCAGTTTTGCAATGATCCCTTCCTGGG |
| | AGTGTACTATCACAAGAATAACAAGTCCTGGATGGAGAGCG |
| | AATTTCGGGTCTACAGCAGCGCAAACAACTGCACCTTCGAGT |
| | ACGTGAGTCAACCCTTTCTGATGGACCTGGAAGGGAAACAG |
| | GGAAACTTCAAGAACCTGAGAGAGTTTGTCTTTAAGAACAT |
| | CGACGGCTATTTTAAGATCTATAGTAAGCATACGCCTATCAA |
| | CCTGGTAAGGGATCTTCCCCAGGGCTTTTCAGCCCTGGAACC |
| | TTTGGTTGACTTGCCTATTGGTATCAATATCACCAGATTTCA |
| | GACCCTTCTGGCATTGCATCGGTCTTATCTTACTCCAGGTGA |
| | TTCCTCCTCCGGGTGGACTGCCGGCGCCGCTGCCTACTATGT |
| | CGGCTATCTGCAACCAAGAACGTTCCTGCTCAAGTACAACG |
| | AAAACGGCACTATTACGGATGCTGTTGATTGTGCCCTGGACC |
| | CTCTGTCTGAGACTAAATGCACCCTCAAGAGCTTTACCGTTG |
| | AGAAGGGGATTTACCAAACCAGTAATTTCCGGGTCCAACCC |
| | ACCGAAAGCATTGTGCGGTTCCCAAATATCACCAATCTGTGT |
| | CCCTTTGGCGAAGTGTTCAATGCTACAAGGTTTGCTTCTGTG |
| | TACGCATGGAATAGGAAACGCATCTCCAATTGTGTCGCTGAT |
| | TACTCCGTGCTGTACAATTCCGCCTCTTTCTCAACCTTCAAGT |
| | GTTATGGCGTTTCACCTACCAAACTTAACGACCTGTGCTTCA |
| | CTAATGTGTATGCCGACTCTTTTGTGATACGAGGCGATGAAG |
| | TGAGACAGATTGCACCAGGGCAGACCGGCAAAATTGCCGAC |
| | TACAACTACAAGCTTCCAGATGACTTTACCGGATGTGTTATT |
| | GCATGGAACTCAAACAATCTGGATTCCAAGGTGGGTGGCAA |
| | CTATAACTACCTGTATAGACTGTTCAGGAAATCCAACCTGAA |
| | ACCATTCGAGCGAGATATAAGCACAGAAATCTACCAGGCTG |
| | GAAGTACGCCCTGCAACGGCGTGGAAGGGTTCAACTGCTAC |
| | TTCCCATTGCAGAGTTACGGATTCCAGCCTACAAACGGGGTG |
| | GGTTACCAACCCTATCGTGTCGTAGTCCTGAGTTTTGAGCTC |
| | CTCCATGCCCCAGCCACAGTCTGTGGCCCCAAGAAAAGCAC |
| | CAATCTGGTGAAGAACAAATGCGTGAACTTTAACTTTAACG |
| | GACTCACAGGAACCGGCGTATTGACGGAGAGTAACAAGAAG |
| | TTCCTGCCATTCCAGCAGTTCGGTCGCGATATTGCCGACACT |
| | ACCGACGCTGTCCGAGATCCCCAGACATTGGAGATTCTTGAT |
| | ATCACACCCTGTAGTTTCGGCGGAGTGAGCGTGATTACGCCC |
| | GGAACCAATACCAGCAATCAGGTTGCCGTCCTGTATCAGGA |
| | CGTGAATTGCACCGAGGTACCTGTCGCCATCCACGCTGACCA |
| | ACTTACACCCACATGGCGAGTATATTCCACCGGCTCCAACGT |
| | CTTTCAGACACGTGCTGGATGTCTGATCGGTGCAGAACACGT |
| | TAATAATAGCTACGAGTGTGATATCCCCATCGGTGCTGGAAT |
| | ATGCGCCTCTTATCAAACTCAAACCAACTCTCCTAGGCGGGC |
| | ACGT |
| SEQ ID NO: 40 | VNLTTRTQLPPAYTNSFTRGVYYPDKVFRSSVLHSTQDLFLPFF |
| SARS-CoV-2 | SNVTWFHAIHVSGTNGTKRFDNPVLPFNDGVYFASTEKSNIIRG |
| D614G Spike | WIFGTTLDSKTQSLLIVNNATNVVIKVCEFQFCNDPFLGVYYHK |
| Truncated S1 | NNKSWMESEFRVYSSANNCTFEYVSQPFLMDLEGKQGNFKNL |
| (amino acid | REFVFKNIDGYFKIYSKHTPINLVRDLPQGFSALEPLVDLPIGINI |
| sequence) | TRFQTLLALHRSYLTPGDSSSGWTAGAAAYYVGYLQPRTFLLK |
| | YNENGTITDAVDCALDPLSETKCTLKSFTVEKGIYQTSNFRVQP |
| | TESIVRFPNITNLCPFGEVENATRFASVYAWNRKRISNCVADYS |
| | VLYNSASFSTFKCYGVSPTKLNDLCFTNVYADSFVIRGDEVRQI |
| | APGQTGKIADYNYKLPDDFTGCVIAWNSNNLDSKVGGNYNYL |
| | YRLFRKSNLKPFERDISTEIYQAGSTPCNGVEGENCYFPLQSYGF |
| | QPTNGVGYQPYRVVVLSFELLHAPATVCGPKKSTNLVKNKCV |
| | NFNFNGLTGTGVLTESNKKFLPFQQFGRDIADTTDAVRDPQTLE |
| | ILDITPCSFGGVSVITPGTNTSNQVAVLYQDVNCTEVPVAIHAD |
| | QLTPTWRVYSTGSNVFQTRAGCLIGAEHVNNSYECDIPIGAGIC |
| | ASYQTQTNSPRRAR |
| SEQ ID NO: 41 | CCCCTCTCCCTCCCCCCCCCCTAACGTTACTGGCCGAAGCCG |
| IRES Bridge | CTTGGAATAAGGCCGGTGTGCGTTTGTCTATATGTTATTTTCC |
| (nucleic acid | ACCATATTGCCGTCTTTTGGCAATGTGAGGGCCCGGAAACCT |
| sequence) | GGCCCTGTCTTCTTGACGAGCATTCCTAGGGGTCTTTCCCCTC |
| | TCGCCAAAGGAATGCAAGGTCTGTTGAATGTCGTGAAGGAA |
| | GCAGTTCCTCTGGAAGCTTCTTGAAGACAAACAACGTCTGTA |
| | GCGACCCTTTGCAGGCAGCGGAACCCCCCACCTGGCGACAG |
| | GTGCCTCTGCGGCCAAAAGCCACGTGTATAAGATACACCTG |
| | CAAAGGCGGCACAACCCCAGTGCCACGTTGTGAGTTGGATA |
| | GTTGTGGAAAGAGTCAAATGGCTCTCCTCAAGCGTATTCAAC |
| | AAGGGGCTGAAGGATGCCCAGAAGGTACCCCATTGTATGGG |
| | ATCTGATCTGGGGCCTCGGTGCACATGCTTTACATGTGTTTA |
| | GTCGAGGTTAAAAAAACGTCTAGGCCCCCCGAACCACGGGG |
| | ACGTGGTTTTCCTTTGAAAAACACGA |
| SEQ ID NO: 42 | ATGTGTCAATCACGCTACCTCCTCTTTTTGGCCACCCTTGCCC |
| Mouse IL-12 | TCCTAAACCACCTCAGTTTGGCCAGGGTCATTCCAGTCTCTG |
| p35 (nucleic acid | GACCTGCCAGGTGTCTTAGCCAGTCCCGAAACCTGCTGAAG |
| sequence) | ACCACAGATGACATGGTGAAGACGGCCAGAGAAAAACTGA |
| | AACATTATTCCTGCACTGCTGAAGACATCGATCATGAAGACA |
| | TCACACGGGACCAAACCAGCACATTGAAGACCTGTTTACCA |
| | CTGGAACTACACAAGAACGAGAGTTGCCTGGCTACTAGAGA |
| | GACTTCTTCCACAACAAGAGGGAGCTGCCTGCCCCCACAGA |
| | AGACGTCTTTGATGATGACCCTGTGCCTTGGTAGCATCTATG |
| | AGGACTTGAAGATGTACCAGACAGAGTTCCAGGCCATCAAC |
| | GCAGCACTTCAGAATCACAACCATCAGCAGATCATTCTAGA |
| | CAAGGGCATGCTGGTGGCCATCGATGAGCTGATGCAGTCTCT |
| | GAATCATAATGGCGAGACTCTGCGCCAGAAACCTCCTGTGG |
| | GAGAAGCAGACCCTTACAGAGTGAAAATGAAGCTCTGCATC |
| | CTGCTTCACGCCTTCAGCACCCGCGTCGTGACCATCAACAGG |
| | GTGATGGGCTATCTGAGCTCCGCCTGA |
| SEQ ID NO: 43 | MCQSRYLLFLATLALLNHLSLARVIPVSGPARCLSQSRNLLKTT |
| Mouse IL-12 | DDMVKTAREKLKHYSCTAEDIDHEDITRDQTSTLKTCLPLELH |
| p35 (amino acid | KNESCLATRETSSTTRGSCLPPQKTSLMMTLCLGSIYEDLKMYQ |
| sequence) | TEFQAINAALQNHNHQQIILDKGMLVAIDELMQSLNHNGETLR |
| | QKPPVGEADPYRVKMKLCILLHAFSTRVVTINRVMGYLSSA |
| SEQ ID NO: 44 | ATGTGTCCTCAGAAGCTAACCATCTCCTGGTTTGCCATCGTT |
| Mouse IL-12 | TTGCTGGTGTCTCCACTCATGGCCATGTGGGAGCTGGAGAAA |
| p40 (nucleic acid | GACGTTTATGTTGTAGAGGTGGACTGGACTCCCGATGCCCCT |
| sequence) | GGAGAAACAGTGAACCTCACCTGTGACACGCCTGAAGAAGA |
| | TGACATCACCTGGACCTCAGACCAGAGACATGGAGTCATAG |
| | GCTCTGGAAAGACCCTGACCATCACTGTCAAAGAGTTTCTAG |
| | ATGCTGGCCAGTACACCTGCCACAAAGGAGGCGAGACTCTG |
| | AGCCACTCACATCTGCTGCTCCACAAGAAGGAAAATGGAAT |
| | TTGGTCCACTGAAATTTTAAAAAATTTCAAAAACAAGACTTT |
| | CCTGAAGTGTGAAGCACCAAATTACTCCGGACGGTTCACGT |
| | GCTCATGGCTGGTGCAAAGAAACATAGACTTGAAGTTCAAC |
| | ATCAAGAGCAGTAGCAGTTCCCCTGACTCTCGGGCAGTGAC |
| | ATGTGGAATGGCGTCTCTGTCTGCAGAGAAGGTCACACTGG |
| | ACCAAAGGGACTATGAGAAGTATTCAGTGTCCTGCCAGGAG |
| | GATGTCACCTGCCCAACTGCCGAGGAGACCCTGCCCATTGA |
| | ACTGGCGTTGGAAGCACGGCAGCAGAATAAATATGAGAACT |
| | ACAGCACCAGCTTCTTCATCAGGGACATCATCAAACCAGAC |
| | CCGCCCAAGAACTTGCAGATGAAGCCTTTGAAGAACTCACA |
| | GGTGGAGGTCAGCTGGGAGTACCCTGACTCCTGGAGCACTC |
| | CCCATTCCTACTTCTCCCTCAAGTTCTTTGTTCGAATCCAGCG |
| | CAAGAAAGAAAAGATGAAGGAGACAGAGGAGGGGTGTAAC |
| | CAGAAAGGTGCGTTCCTCGTAGAGAAGACATCTACCGAAGT |
| | CCAATGCAAAGGCGGGAATGTCTGCGTGCAAGCTCAGGATC |
| | GCTATTACAATTCCTCATGCAGCAAGTGGGCATGTGTTCCCT |
| | GCAGGGTCCGATCCTAG |
| SEQ ID NO: 45 | MCPQKLTISWFAIVLLVSPLMAMWELEKDVYVVEVDWTPDAP |
| Mouse IL-12 | GETVNLTCDTPEEDDITWTSDQRHGVIGSGKTLTITVKEFLDAG |
| p40 (amino acid | QYTCHKGGETLSHSHLLLHKKENGIWSTEILKNFKNKTFLKCE |
| sequence) | APNYSGRFTCSWLVQRNIDLKFNIKSSSSSPDSRAVTCGMASLS |
| | AEKVTLDQRDYEKYSVSCQEDVTCPTAEETLPIELALEARQQN |
| | KYENYSTSFFIRDIIKPDPPKNLQMKPLKNSQVEVSWEYPDSWS |
| | TPHSYFSLKFFVRIQRKKEKMKETEEGCNQKGAFLVEKTSTEV |
| | QCKGGNVCVQAQDRYYNSSCSKWACVPCRVRS |
| SEQ ID NO: 46 | ATGGGTCCAGCGCGCAGCCTCCTCCTTGTGGCTACCCTGGTC |
| Human IL-12 | CTCCTGGACCACCTCAGTTTGGCCAGAAACCTCCCCGTGGCC |
| p35 (nucleic acid | ACTCCAGACCCAGGAATGTTCCCATGCCTTCACCACTCCCAA |
| sequence) | AACCTGCTGAGGGCCGTCAGCAACATGCTCCAGAAGGCCAG |
| | ACAAACTCTAGAATTTTACCCTTGCACTTCTGAAGAGATTGA |
| | TCATGAAGATATCACAAAAGATAAAACCAGCACAGTGGAGG |
| | CCTGTTTACCATTGGAATTAACCAAGAATGAGAGTTGCCTAA |
| | ATTCCAGAGAGACCTCTTTCATAACTAATGGGAGTTGCCTGG |
| | CCTCCAGAAAGACCTCTTTTATGATGGCCCTGTGCCTTAGTA |
| | GTATTTATGAAGACTTGAAGATGTACCAGGTGGAGTTCAAG |
| | ACCATGAATGCAAAGCTTCTGATGGATCCTAAGAGGCAGAT |
| | CTTTCTAGATCAAAACATGCTGGCAGTTATTGATGAGCTGAT |
| | GCAGGCCCTGAATTTCAACAGTGAGACTGTGCCACAAAAAT |
| | CCTCCCTTGAAGAACCGGATTTTTATAAAACTAAAATCAAGC |
| | TCTGCATACTTCTTCATGCTTTCAGAATTCGGGCAGTGACTA |
| | TTGATAGAGTGATGAGCTATCTGAATGCTTCCTAA |
| SEQ ID NO: 47 | MGPARSLLLVATLVLLDHLSLARNLPVATPDPGMFPCLHHSQN |
| Human IL-12 | LLRAVSNMLQKARQTLEFYPCTSEEIDHEDITKDKTSTVEACLP |
| p35 (amino acid | LELTKNESCLNSRETSFITNGSCLASRKTSFMMALCLSSIYEDLK |
| sequence) | MYQVEFKTMNAKLLMDPKRQIFLDQNMLAVIDELMQALNENS |
| | ETVPQKSSLEEPDFYKTKIKLCILLHAFRIRAVTIDRVMSYLNAS |
| SEQ ID NO: 48 | ATGGGTCACCAGCAGTTGGTCATCTCTTGGTTTTCCCTGGTTT |
| Human IL-12 | TTCTGGCATCTCCCCTCGTGGCCATATGGGAACTGAAGAAAG |
| p40 (nucleic acid | ATGTTTATGTCGTAGAATTGGATTGGTATCCGGATGCCCCTG |
| sequence) | GAGAAATGGTGGTCCTCACCTGTGACACCCCTGAAGAAGAT |
| | GGTATCACCTGGACCTTGGACCAGAGCAGTGAGGTCTTAGG |
| | CTCTGGCAAAACCCTGACCATCCAAGTCAAAGAGTTTGGAG |
| | ATGCTGGCCAGTACACCTGTCACAAAGGAGGCGAGGTTCTA |
| | AGCCATTCGCTCCTGCTGCTTCACAAAAAGGAAGATGGAATT |
| | TGGTCCACTGATATTTTAAAGGACCAGAAAGAACCCAAAAA |
| | TAAGACCTTTCTAAGATGCGAGGCCAAGAATTATTCTGGACG |
| | TTTCACCTGCTGGTGGCTGACGACAATCAGTACTGATTTGAC |
| | ATTCAGTGTCAAAAGCAGCAGAGGCTCTTCTGACCCCCAAG |
| | GGGTGACGTGCGGAGCTGCTACACTCTCTGCAGAGAGAGTC |
| | AGAGGGGACAACAAGGAGTATGAGTACTCAGTGGAGTGCCA |
| | GGAGGACAGTGCCTGCCCAGCTGCTGAGGAGAGTCTGCCCA |
| | TTGAGGTCATGGTGGATGCCGTTCACAAGCTCAAGTATGAA |
| | AACTACACCAGCAGCTTCTTCATCAGGGACATCATCAAACCT |
| | GACCCACCCAAGAACTTGCAGCTGAAGCCATTAAAGAATTC |
| | TCGGCAGGTGGAGGTCAGCTGGGAGTACCCTGACACCTGGA |
| | GTACTCCACATTCCTACTTCTCCCTGACATTCTGCGTTCAGGT |
| | CCAGGGCAAGAGCAAGAGAGAAAAGAAAGATAGAGTCTTC |
| | ACGGACAAGACCTCAGCCACGGTCATCTGCCGCAAAAATGC |
| | CAGCATTAGCGTGCGGGCCCAGGACCGCTACTATAGCTCATC |
| | TTGGAGCGAATGGGCATCTGTGCCCTGCAGTTAGAC |
| SEQ ID NO: 49 | MGHQQLVISWFSLVFLASPLVAIWELKKDVYVVELDWYPDAP |
| Human IL-12 | GEMVVLTCDTPEEDGITWTLDQSSEVLGSGKTLTIQVKEFGDA |
| p40 (amino acid | GQYTCHKGGEVLSHSLLLLHKKEDGIWSTDILKDQKEPKNKTF |
| sequence) | LRCEAKNYSGRFTCWWLTTISTDLTFSVKSSRGSSDPQGVTCG |
| | AATLSAERVRGDNKEYEYSVECQEDSACPAAEESLPIEVMVDA |
| | VHKLKYENYTSSFFIRDIIKPDPPKNLQLKPLKNSRQVEVSWEY |
| | PDTWSTPHSYFSLTFCVQVQGKSKREKKDRVFTDKTSATVICR |
| | KNASISVRAQDRYYSSSWSEWASVPCS |
| SEQ ID NO: 51 | ACCGGCGCCTACCTGAGATCACCGGTAACGTAGATAAGCGC |
| AgeIMhis-F | TTGCCACAACCCGGGATC |
| SEQ ID NO: 52 | TATTTGAATGTATTTAGAAAAATAAACAAATAGGGAAATGA |
| pA-R | AGAGCCCATACCACATTTGTAGAGGTTTTAC |
| SEQ ID NO: 53 | ACCGGCGCCTACCTGAGATCACCGGTGCCGCCACCATGGAG |
| AgeIsp1-F | ATCAAGGTGCTGTTTG |
| SEQ ID NO: 54 | TGAGGGAAGCGGCCGCCCGGGTCGACTCTAGAGGATCCCG |
| IRESSalI-R | GGTTGTGGCAAGCTTATCATCGTGTTTTTCAAAGGAAAAC |
| SEQ ID NO: 56 | CGGCTAAGCGGCCGCAGCCTCGAGCAGCTAGCTGGCCAGAC |
| PspXI-SV40pA- | |
| F | |
| SEQ ID NO: 57 | TATTTGAATGTATTTAGAAAAATAAACAAATAGG |
| Cls-SapI-R | |
| SEQ ID NO: 58 | ATTGGACATGAGCCAATATAAATGTACGAAGATCTGTCGAG |
| Cls-BglII-R-2 | CCATGTG |
| SEQ ID NO: 59 | AGAACTTTAAAAGTGCTCATCATTG |
| Cls-XmnI-R | |
| SEQ. ID NO. 60 | ATACGTAAGCGGCCGCAGCCTCG |
| CLS-PspXI- | |
| IRES-F | |
| SEQ. ID NO. 61 | TCGTGTTTTTCAAAGGAAAACCAC |
| IRES-R | |
| SEQ. ID NO. 62 | ACGTGGTTTTCCTTTGAAAAACACGATGATAAGCTTGCCACA |
| Mhis-F | ACCCG |
| SEQ. ID NO. 63 | ATCATGTCTGGCCAGCTAGC |
| CLS-M-R | |
| SEQ. ID NO. 64 | CTAGACTCGAGACAGTCCACCACAATCTAGAGCCACCATGG |
| CLS-XbaI-M-F | CTGACTCT |
| SEQ. ID NO. 65 | ACTCATCAATGTATCTTATCATGTCTGCTCGAacgcgtttattgtac |
| CLS-MluI-M-R | |
| SEQ ID NO. 66 | ATGGAACTTTTGATACTCAAGGCCAATGCTATAACTACCATT |
| RSV F nucleic | TTAACCGCTGTCACGTTTTGTTTCGCTTCTGGCCAAAATATTA |
| acid sequence | CCGAAGAATTCTATCAATCTACTTGCTCCGCAGTGAGCAAAG |
| | GTTACTTATCTGCTTTGAGAACAGGATGGTACACATCCGTGA |
| | TAACAATTGAGCTCTCTAATATAAAGGAGAACAAGTGTAAT |
| | GGTACCGATGCAAAGGTCAAACTTATAAAGCAAGAGCTGGA |
| | TAAATACAAGAATGCAGTAACTGAGTTGCAACTGCTGATGC |
| | AATCTACTCCTGCTACTAATAATAGAGCGAGACGGGAACTTC |
| | CCAGGTTCATGAACTACACACTTAATAATGCTAAGAAAACT |
| | AACGTTACGCTCAGTAAGAAGCGGAAGCGCCGTTTCTTAGG |
| | ATTTCTCCTTGGCGTTGGCAGTGCCATAGCTTCCGGAGTCGC |
| | AGTCTCTAAAGTCTTGCACCTTGAAGGTGAAGTCAACAAAAT |
| | TAAATCAGCACTGTTAAGCACCAATAAGGCAGTCGTATCTCT |
| | GAGTAATGGCGTCTCTGTGCTCACTTCCAAGGTCCTGGACCT |
| | TAAGAACTACATAGACAAACAATTACTCCCGATTGTGAACA |
| | AACAGAGCTGCAGCATATCAAATATAGAAACCGTGATCGAA |
| | TTCCAACAGAAGAATAACCGTTTACTGGAGATAACGAGGGA |
| | ATTCTCCGTAAATGCCGGTGTCACAACTCCTGTGTCTACTTA |
| | TATGTTGACCAACAGCGAACTGCTGTCACTTATAAACGACAT |
| | GCCCATCACTAATGACCAAAAGAAGCTTATGTCCAACAACG |
| | TTCAAATCGTCAGACAACAGAGCTATTCTATCATGTCTATAA |
| | TAAAAGAGGAAGTTCTTGCATACGTCGTACAACTGCCATTGT |
| | ACGGTGTGATCGACACTCCCTGCTGGAAATTGCATACATCTC |
| | CGCTTTGTACAACAAATACGAAGGAAGGCAGTAATATATGT |
| | TTGACCCGTACAGATCGTGGTTGGTATTGCGATAACGCCGGG |
| | TCCGTGTCCTTCTTCCCCCAAGCAGAAACCTGTAAAGTCCAG |
| | TCTAACAGGGTCTTCTGTGACACCATGAATAGCCTCACCTTA |
| | CCATCCGAGGTGAACTTATGTAATGTTGATATTTTCAATCCC |
| | AAGTATGACTGTAAAATAATGACCAGTAAAACCGACGTTAG |
| | CAGTAGTGTGATTACCTCTTTGGGTGCAATTGTTAGCTGTTA |
| | CGGTAAAACAAAGTGCACAGCGTCCAACAAGAATCGCGGCA |
| | TTATCAAGACATTCTCTAACGGATGTGACTATGTCAGCAACA |
| | AAGGAGTGGATACGGTCTCTGTGGGCAACACCCTCTACTAC |
| | GTCAACAAACAAGAGGGGAAGAGCTTATATGTGAAAGGCGA |
| | ACCTATCATCAACTTTTACGATCCACTTGTATTTCCTAGCGAT |
| | GAGTTCGATGCCAGTATCTCTCAAGTAAACGAAAAGATAAA |
| | TCAGTCTCTGGCGTTCATTCGCAAAAGCGACGAGCTTTTAGG |
| | TGGGGGGGGAAGCGGTTACATTCCCGAGGCCCCACGGGACG |
| | GCCAGGCTTATGTTCGCAAGGATGGAGAATGGGTGCTTTTGA |
| | GTACGTTCCTTTAA |
| SEQ ID NO. 67 | MELLILKANAITTILTAVTFCFASGQNITEEFYQSTCSAVSKGYL |
| RSV F amino | SALRTGWYTSVITIELSNIKENKCNGTDAKVKLIKQELDKYKNA |
| acid sequence | VTELQLLMQSTPATNNRARRELPRFMNYTLNNAKKTNVTLSK |
| | KRKRRFLGFLLGVGSAIASGVAVSKVLHLEGEVNKIKSALLSTN |
| | KAVVSLSNGVSVLTSKVLDLKNYIDKQLLPIVNKQSCSISNIETV |
| | IEFQQKNNRLLEITREFSVNAGVTTPVSTYMLTNSELLSLINDMP |
| | ITNDQKKLMSNNVQIVRQQSYSIMSIIKEEVLAYVVQLPLYGVI |
| | DTPCWKLHTSPLCTTNTKEGSNICLTRTDRGWYCDNAGSVSFF |
| | PQAETCKVQSNRVFCDTMNSLTLPSEVNLCNVDIFNPKYDCKI |
| | MTSKTDVSSSVITSLGAIVSCYGKTKCTASNKNRGIIKTFSNGCD |
| | YVSNKGVDTVSVGNTLYYVNKQEGKSLYVKGEPIINFYDPLVF |
| | PSDEFDASISQVNEKINQSLAFIRKSDELLGGGGSGYIPEAPRDG |
| | QAYVRKDGEWVLLSTFL* |
| SEQ ID NO. 68 | ATGGAGAGCAGGATCTGGTGTTTAGTTGTTTGTGTTAATCTG |
| CMV gB nucleic | TGCATTGTTTGTCTGGGTGCAGCAGTTAGTTCAAGTTCCACA |
| acid sequence | CGCGGCACATCAGCCACTCATAGTCACCATAGTTCCCATACC |
| | ACCAGCGCAGCCCATAGTAGGTCTGGTTCAGTGTCTCAGCGG |
| | GTGACCTCAAGCCAAACGGTCTCACACGGGGTCAATGAGAC |
| | AATTTATAATACAACACTCAAGTACGGGGATGTAGTAGGTG |
| | TAAACACTACCAAGTATCCATACAGAGTCTGTTCTATGGCAC |
| | AGGGCACAGATCTTATAAGGTTTGAACGAAATATTGTATGTA |
| | CGTCCATGAAACCCATTAATGAGGACCTCGACGAAGGAATC |
| | ATGGTGGTCTACAAGCGGAATATCGTGGCCCATACATTCAA |
| | AGTACGTGTTTACCAGAAAGTCCTCACTTTCAGACGGTCCTA |
| | CGCTTATATTCATACCACATATCTTCTGGGTTCCAATACTGA |
| | ATATGTCGCACCACCTATGTGGGAAATCCATCATATAAACTC |
| | CCACTCACAATGCTATAGCTCCTATTCCAGAGTTATTGCTGG |
| | GACAGTGTTCGTGGCTTATCACAGGGATAGTTATGAAAATA |
| | AGACCATGCAACTGATGCCGGACGATTACAGTAACACACAC |
| | TCCACCCGTTACGTAACCGTCAAGGATCAGTGGCACTCTCGC |
| | GGCAGTACATGGCTGTACCGTGAGACTTGCAACTTGAACTGT |
| | ATGGTCACTATTACCACTGCAAGGTCCAAATATCCCTATCAT |
| | TTCTTCGCGACGTCTACCGGAGATGTGGTTGACATATCCCCG |
| | TTTTATAACGGTACAAACCGCAACGCCAGTTACTTCGGAGAA |
| | AATGCCGATAAGTTCTTCATCTTCCCCAACTACACAATAGTG |
| | TCAGACTTTGGGCGCCCGAATAGCGCCCTCGAAACTCACCGC |
| | TTGGTCGCCTTTCTTGAGAGAGCAGATTCTGTCATCTCTTGG |
| | GACATTCAGGACGAAAAGAACGTCACTTGTCAGTTAACCTTC |
| | TGGGAAGCGTCTGAGCGCACCATCAGATCTGAGGCGGAAGA |
| | TTCTTACCATTTTAGCTCAGCCAAGATGACCGCTACCTTTCTC |
| | AGCAAGAAGCAAGAGGTGAACATGAGCGATAGCGCTCTGGA |
| | CTGCGTAAGAGATGAAGCAATCAACAAGTTACAGCAAATAT |
| | TTAACACCAGTTACAACCAAACGTACGAGAAGTATGGGAAC |
| | GTATCCGTTTTCGAGACAACCGGCGGACTTGTGGTCTTTTGG |
| | CAGGGTATTAAACAAAAGTCTTTAGTCGAACTGGAAAGACT |
| | GGCGAACAGATCCAGCCTGAACCTGACACACAATCGCACTA |
| | AACGTTCAACTGATGGAAATAATGCAACCCATCTCAGCAAC |
| | ATGGAGAGTGTGCACAACCTTGTCTACGCTCAGTTACAATTC |
| | ACTTATGACACCCTGCGAGGTTACATTAATCGAGCTCTGGCA |
| | CAAATCGCAGAGGCATGGTGCGTTGATCAACGCCGGACACT |
| | GGAAGTGTTTAAGGAATTGAGTAAGATTAATCCCAGTGCAA |
| | TCTTGTCCGCGATCTATAACAAGCCCATTGCAGCTCGTTTCA |
| | TGGGAGATGTCCTCGGATTAGCTTCCTGCGTCACTATTAATC |
| | AAACATCCGTTAAAGTCCTGAGAGACATGAATGTTAAAGAG |
| | TCACCGGGACGGTGTTATAGTCGGCCCGTGGTCATATTCAAT |
| | TTTGCCAACTCTAGCTACGTCCAGTATGGCCAATTGGGTGAA |
| | GATAATGAAATCTTATTGGGGAATCACAGGACCGAAGAATG |
| | CCAACTCCCCAGCTTGAAGATCTTTATAGCTGGTAACTCAGC |
| | CTACGAGTATGTCGACTATCTCTTCAAAAGGATGATCGATCT |
| | CTCAAGTATCAGTACGGTCGACTCAATGATCGCTCTCGACAT |
| | AGACCCGCTCGAAAACACTGATTTCCGGGTGCTGGAACTCTA |
| | CTCACAAAAGGAACTCCGGTCATCAAATGTCTTTGATCTGGA |
| | AGAGATCATGCGCGAGTTTAATTCCTACAAGCAAAGGGTCA |
| | AATACGTTGAAGACAAGGTCGTGGATCCTCTGCCCCCATATT |
| | TGAAAGGATTAGACGATCTCATGTCTGGACTTGGAGCCGCC |
| | GGAAAAGCAGTTGGTGTAGCTATCGGGGCAGTTGGCGGTGC |
| | TGTTGCTAGTGTCGTGGAAGGGGTGGCGACCTTTCTGAAGAA |
| | CCCCTTTGGCGCATTCACGATAATCTTAGTTGCGATCGCAGT |
| | TGTTATCATTATTTACTTAATATATACTAGACAAAGACGCCT |
| | GTGTATGCAACCCCTGCAAAATCTGTTTCCGTATCTCGTATC |
| | TGCCGACGGGACCACCGTGACGAGTGGGAACACCAAAGACA |
| | CTTCCCTGCAAGCTCCCCCATCTTACGAAGAAAGCGTGTATA |
| | ATAGCGGTCGAAAAGGGCCTGGACCCCCATCAAGTGATGCC |
| | TCTACTGCTGCTCCCCCTTATACTAATGAACAAGCCTACCAA |
| | ATGCTGCTCGCCCTGGTTCGGTTAGACGCCGAACAACGGGC |
| | GCAACAGAACGGTACTGATTCACTTGACGGACAAACCGGGA |
| | CACAAGATAAAGGGCAAAAGCCCAACTTACTGGACAGATTG |
| | AGACACAGAAAGAACGGGTACCGGCATCTTAAGGACAGCGA |
| | TGAGGAAGAGAATGTGTGA |
| SEQ ID NO. 69 | MESRIWCLVVCVNLCIVCLGAAVSSSSTRGTSATHSHHSSHTTS |
| CMV gB amino | AAHSRSGSVSQRVTSSQTVSHGVNETIYNTTLKYGDVVGVNTT |
| acid sequence | KYPYRVCSMAQGTDLIRFERNIVCTSMKPINEDLDEGIMVVYK |
| | RNIVAHTFKVRVYQKVLTFRRSYAYIHTTYLLGSNTEYVAPPM |
| | WEIHHINSHSQCYSSYSRVIAGTVFVAYHRDSYENKTMQLMPD |
| | DYSNTHSTRYVTVKDQWHSRGSTWLYRETCNLNCMVTITTAR |
| | SKYPYHFFATSTGDVVDISPFYNGTNRNASYFGENADKFFIFPN |
| | YTIVSDFGRPNSALETHRLVAFLERADSVISWDIQDEKNVTCQL |
| | TFWEASERTIRSEAEDSYHFSSAKMTATFLSKKQEVNMSDSAL |
| | DCVRDEAINKLQQIFNTSYNQTYEKYGNVSVFETTGGLVVFWQ |
| | GIKQKSLVELERLANRSSLNLTHNRTKRSTDGNNATHLSNMES |
| | VHNLVYAQLQFTYDTLRGYINRALAQIAEAWCVDQRRTLEVF |
| | KELSKINPSAILSAIYNKPIAARFMGDVLGLASCVTINQTSVKVL |
| | RDMNVKESPGRCYSRPVVIFNFANSSYVQYGQLGEDNEILLGN |
| | HRTEECQLPSLKIFIAGNSAYEYVDYLFKRMIDLSSISTVDSMIA |
| | LDIDPLENTDFRVLELYSQKELRSSNVFDLEEIMREFNSYKQRV |
| | KYVEDKVVDPLPPYLKGLDDLMSGLGAAGKAVGVAIGAVGG |
| | AVASVVEGVATFLKNPFGAFTIILVAIAVVIIIYLIYTRQRRLCM |
| | QPLQNLFPYLVSADGTTVTSGNTKDTSLQAPPSYEESVYNSGRK |
| | GPGPPSSDASTAAPPYTNEQAYQMLLALVRLDAEQRAQQNGT |
| | DSLDGQTGTQDKGQKPNLLDRLRHRKNGYRHLKDSDEEENV* |
| SEQ ID NO. 70 | ATGAAGGTGAAACTGCTGGTGCTGCTGTGTACCTTCACAGCC |
| H1N1 2007 | ACCTATGCTGACACCATCTGTATTGGCTACCATGCCAACAAC |
| Brisbane HA | AGCACAGACACAGTGGACACAGTGTTGGAGAAGAATGTGAC |
| Nucleic Acid | AGTGACCCACTCTGTGAACCTGTTGGAGAACAGCCACAATG |
| Sequence | GCAAACTGTGTCTGCTGAAAGGCATTGCCCCACTCCAACTTG |
| | GCAACTGTTCTGTGGCTGGCTGGATTCTGGGCAACCCTGAGT |
| | GTGAACTGCTGATTAGCAAGGAGTCCTGGTCCTACATTGTGG |
| | AGAAGCCAAACCCTGAGAATGGCACTTGTTACCCTGGACAC |
| | TTTGCTGACTATGAGGAACTGAGGGAACAACTTTCCTCTGTG |
| | TCCTCCTTTGAGAGGTTTGAGATTTTTCCAAAGGAGTCCTCC |
| | TGGCCAAACCACACAGTGACAGGAGTGTCTGCCTCCTGTAG |
| | CCACAATGGAGAGTCCTCCTTCTACAGGAACCTGCTGTGGCT |
| | GACAGGCAAGAATGGACTCTACCCAAACCTGAGCAAGTCCT |
| | ATGCCAACAACAAGGAGAAGGAGGTGCTGGTGCTGTGGGGA |
| | GTGCATCACCCTCCAAACATTGGAGACCAGAAGGCTCTCTAC |
| | CACACAGAGAATGCCTATGTGTCTGTGGTGTCCAGCCACTAC |
| | AGCAGGAAGTTCACACCTGAGATTGCCAAGAGACCAAAGGT |
| | GAGGGACCAGGAGGGCAGGATAAACTACTACTGGACCCTGT |
| | TGGAACCTGGAGACACCATCATCTTTGAGGCTAATGGCAAC |
| | CTGATTGCCCCAAGATATGCCTTTGCCCTGAGCAGGGGCTTT |
| | GGCTCTGGCATCATCAACAGCAATGCCCCTATGGACAAGTGT |
| | GATGCCAAGTGTCAGACACCACAGGGAGCCATCAACTCCTC |
| | CCTGCCATTCCAGAATGTGCATCCTGTGACCATTGGAGAGTG |
| | TCCAAAATATGTGAGGTCTGCCAAACTGAGGATGGTGACAG |
| | GACTGAGGAACATCCCAAGCATCCAGAGCAGGGGACTGTTT |
| | GGAGCCATTGCTGGCTTCATTGAGGGAGGCTGGACAGGGAT |
| | GGTGGATGGCTGGTATGGCTACCACCACCAGAATGAACAGG |
| | GCTCTGGCTATGCTGCTGACCAGAAGAGCACCCAGAATGCC |
| | ATCAATGGCATCACCAACAAGGTGAACTCTGTGATTGAGAA |
| | GATGAACACCCAGTTCACAGCAGTGGGCAAGGAGTTCAACA |
| | AATTGGAGAGGAGGATGGAGAACCTGAACAAGAAGGTGGA |
| | TGATGGCTTCATTGACATCTGGACCTACAATGCTGAACTGCT |
| | GGTGCTGTTGGAGAATGAGAGGACCCTGGACTTCCATGACA |
| | GCAATGTGAAGAACCTCTATGAGAAGGTGAAGAGCCAACTT |
| | AAAAACAATGCCAAGGAGATTGGCAATGGCTGTTTTGAGTT |
| | CTACCACAAGTGTAATGATGAGTGTATGGAGTCTGTGAAGA |
| | ATGGCACCTATGACTACCCAAAATACTCTGAGGAGAGCAAA |
| | CTGAACAGGGAGAAGATTGATGGAGTGAAATTGGAGAGTAT |
| | GGGAGTCTACCAGATTCTGGCTATCTACAGCACAGTGGCATC |
| | CTCCCTGGTGCTGCTGGTGTCCCTGGGAGCCATCTCCTTCTG |
| | GATGTGTAGCAATGGCTCCCTCCAATGTAGGATTTGTATTTA |
| | A |
| SEQ ID NO. 71 | MKVKLLVLLCTFTATYADTICIGYHANNSTDTVDTVLEKNVTV |
| H1N1 2007 | THSVNLLENSHNGKLCLLKGIAPLQLGNCSVAGWILGNPECELL |
| Brisbane HA | ISKESWSYIVEKPNPENGTCYPGHFADYEELREQLSSVSSFERFE |
| Amino Acid | IFPKESSWPNHTVTGVSASCSHNGESSFYRNLLWLTGKNGLYP |
| sequence | NLSKSYANNKEKEVLVLWGVHHPPNIGDQKALYHTENAYVSV |
| | VSSHYSRKFTPEIAKRPKVRDQEGRINYYWTLLEPGDTIIFEANG |
| | NLIAPRYAFALSRGFGSGIINSNAPMDKCDAKCQTPQGAINSSLP |
| | FQNVHPVTIGECPKYVRSAKLRMVTGLRNIPSIQSRGLFGAIAGF |
| | IEGGWTGMVDGWYGYHHQNEQGSGYAADQKSTQNAINGITN |
| | KVNSVIEKMNTQFTAVGKEFNKLERRMENLNKKVDDGFIDIWT |
| | YNAELLVLLENERTLDFHDSNVKNLYEKVKSQLKNNAKEIGNG |
| | CFEFYHKCNDECMESVKNGTYDYPKYSEESKLNREKIDGVKLE |
| | SMGVYQILAIYSTVASSLVLLVSLGAISFWMCSNGSLQCRICI* |
| SEQ ID NO. 72 | MFVFLVLLPLVSSQCVNLTTRTQLPPAYTNSFTRGVYYPDKVFR |
| SARS-COV-2 | SSVLHSTQDLFLPFFSNVTWFHVISGTNGTKRFDNPVLPFNDGV |
| Omicron BA.1 | YFASIEKSNIIRGWIFGTTLDSKTQSLLIVNNATNVVIKVCEFQFC |
| Amino Acid Seq | NDPFLDHKNNKSWMESEFRVYSSANNCTFEYVSQPFLMDLEG |
| | KQGNFKNLREFVFKNIDGYFKIYSKHTPIIVREPEDLPQGFSALE |
| | PLVDLPIGINITRFQTLLALHRSYLTPGDSSSGWTAGAAAYYVG |
| | YLQPRTFLLKYNENGTITDAVDCALDPLSETKCTLKSFTVEKGI |
| | YQTSNFRVQPTESIVRFPNITNLCPFDEVFNATRFASVYAWNRK |
| | RISNCVADYSVLYNLAPFFTFKCYGVSPTKLNDLCFTNVYADSF |
| | VIRGDEVRQIAPGQTGNIADYNYKLPDDFTGCVIAWNSNKLDS |
| | KVSGNYNYLYRLFRKSNLKPFERDISTEIYQAGNKPCNGVAGF |
| | NCYFPLRSYSFRPTYGVGHQPYRVVVLSFELLHAPATVCGPKK |
| | STNLVKNKCVNFNFNGLKGTGVLTESNKKFLPFQQFGRDIADT |
| | TDAVRDPQTLEILDITPCSFGGVSVITPGTNTSNQVAVLYQGVN |
| | CTEVPVAIHADQLTPTWRVYSTGSNVFQTRAGCLIGAEYVNNS |
| | YECDIPIGAGICASYQTQTKSHRRARSVASQSIIAYTMSLGAENS |
| | VAYSNNSIAIPTNFTISVTTEILPVSMTKTSVDCTMYICGDSTECS |
| | NLLLQYGSFCTQLKRALTGIAVEQDKNTQEVFAQVKQIYKTPPI |
| | KYFGGFNFSQILPDPSKPSKRSFIEDLLFNKVTLADAGFIKQYGD |
| | CLGDIAARDLICAQKFKGLTVLPPLLTDEMIAQYTSALLAGTITS |
| | GWTFGAGAALQIPFAMQMAYRENGIGVTQNVLYENQKLIANQ |
| | FNSAIGKIQDSLSSTASALGKLQDVVNHNAQALNTLVKQLSSKF |
| | GAISSVLNDIFSRLDKVEAEVQIDRLITGRLQSLQTYVTQQLIRA |
| | AEIRASANLAATKMSECVLGQSKRVDFCGKGYHLMSFPQSAPH |
| | GVVFLHVTYVPAQEKNFTTAPAICHDGKAHFPREGVFVSNGTH |
| | WFVTQRNFYEPQIITTDNTFVSGNCDVVIGIVNNTVYDPLQPEL |
| | DSFKEELDKYFKNHTSPDVDLGDISGINASVVNIQKEIDRLNEV |
| | AKNLNESLIDLQELGKYEQYIKWPWYIWLGFIAGLIAIVMVTIM |
| | LCCMTSCCSCLKGCCSCGSCCKFDEDDSEPVLKGVKLHYT |
| SEQ ID NO. 73 | MFVFLVLLPLVSSQCVNLITRTQSYTNSFTRGVYYPDKVFRSSV |
| SARS-CoV-2 | LHSTQDLFLPFFSNVTWFHAIHVSGTNGTKRFDNPVLPFNDGVY |
| Omicron BA.2 | FASTEKSNIIRGWIFGTTLDSKTQSLLIVNNATNVVIKVCEFQFC |
| Amino Acid Seq | NDPFLDVYYHKNNKSWMESEFRVYSSANNCTFEYVSQPFLMD |
| | LEGKQGNFKNLREFVFKNIDGYFKIYSKHTPINLGRDLPQGFSA |
| | LEPLVDLPIGINITRFQTLLALHRSYLTPGDSSSGWTAGAAAYY |
| | VGYLQPRTFLLKYNENGTITDAVDCALDPLSETKCTLKSFTVEK |
| | GIYQTSNFRVQPTESIVRFPNITNLCPFDEVFNATRFASVYAWNR |
| | KRISNCVADYSVLYNFAPFFAFKCYGVSPTKLNDLCFTNVYAD |
| | SFVIRGNEVSQIAPGQTGNIADYNYKLPDDFTGCVIAWNSNKLD |
| | SKVGGNYNYLYRLFRKSNLKPFERDISTEIYQAGNKPCNGVAG |
| | FNCYFPLRSYGFRPTYGVGHQPYRVVVLSFELLHAPATVCGPK |
| | KSTNLVKNKCVNFNFNGLTGTGVLTESNKKFLPFQQFGRDIAD |
| | TTDAVRDPQTLEILDITPCSFGGVSVITPGTNTSNQVAVLYQGV |
| | NCTEVPVAIHADQLTPTWRVYSTGSNVFQTRAGCLIGAEYVNN |
| | SYECDIPIGAGICASYQTQTKSHRRARSVASQSIIAYTMSLGAEN |
| | SVAYSNNSIAIPTNFTISVTTEILPVSMTKTSVDCTMYICGDSTEC |
| | SNLLLQYGSFCTQLKRALTGIAVEQDKNTQEVFAQVKQIYKTPP |
| | IKYFGGFNFSQILPDPSKPSKRSFIEDLLFNKVTLADAGFIKQYG |
| | DCLGDIAARDLICAQKFNGLTVLPPLLTDEMIAQYTSALLAGTI |
| | TSGWTFGAGAALQIPFAMQMAYRFNGIGVTQNVLYENQKLIA |
| | NQFNSAIGKIQDSLSSTASALGKLQDVVNHNAQALNTLVKQLS |
| | SKFGAISSVLNDILSRLDKVEAEVQIDRLITGRLQSLQTYVTQQL |
| | IRAAEIRASANLAATKMSECVLGQSKRVDFCGKGYHLMSFPQS |
| | APHGVVFLHVTYVPAQEKNFTTAPAICHDGKAHFPREGVFVSN |
| | GTHWFVTQRNFYEPQIITTDNTFVSGNCDVVIGIVNNTVYDPLQ |
| | PELDSFKEELDKYFKNHTSPDVDLGDISGINASVVNIQKEIDRLN |
| | EVAKNLNESLIDLQELGKYEQYIKWPWYIWLGFIAGLIAIVMVT |
| | IMLCCMTSCCSCLKGCCSCGSCCKFDEDDSEPVLKGVKLHYT |
| SEQ ID NO. 74 | MFVFLVLLPLVSSQCVNLRTRTQLPPAYTNSFTRGVYYPDKVF |
| SARS-CoV-2 | RSSVLHSTQDLFLPFFSNVTWFHAIHVSGTNGTKRFDNPVLPFN |
| Delta Variant | DGVYFASIEKSNIIRGWIFGTTLDSKTQSLLIVNNATNVVIKVCE |
| Amino Acid Seq | FQFCNDPFLDVYYHKNNKSWMESGVYSSANNCTFEYVSQPFL |
| | MDLEGKQGNFKNLREFVFKNIDGYFKIYSKHTPINLVRDLPQGF |
| | SALEPLVDLPIGINITRFQTLLALHRSYLTPGDSSSGWTAGAAAY |
| | YVGYLQPRTFLLKYNENGTITDAVDCALDPLSETKCTLKSFTVE |
| | KGIYQTSNFRVQPTESIVRFPNITNLCPFGEVENATRFASVYAW |
| | NRKRISNCVADYSVLYNSASFSTFKCYGVSPTKLNDLCFTNVY |
| | ADSFVIRGDEVRQIAPGQTGKIADYNYKLPDDFTGCVIAWNSN |
| | NLDSKVGGNYNYRYRLFRKSNLKPFERDISTEIYQAGSKPCNG |
| | VEGFNCYFPLQSYGFQPTNGVGYQPYRVVVLSFELLHAPATVC |
| | GPKKSTNLVKNKCVNFNFNGLTGTGVLTESNKKFLPFQQFGRD |
| | IADTTDAVRDPQTLEILDITPCSFGGVSVITPGTNTSNQVAVLYQ |
| | GVNCTEVPVAIHADQLTPTWRVYSTGSNVFQTRAGCLIGAEHV |
| | NNSYECDIPIGAGICASYQTQTNSRRRARSVASQSIIAYTMSLGE |
| | ENSVAYSNNSIAIPTNFTISVTTEILPVSMTKTSVDCTMYICGDST |
| | ECSNLLLQYGSFCTQLNRALTGIAVEQDKNTQEVFAQVKQIYK |
| | TPPIKDFGGFNFSQILPDPSKPSKRSFIEDLLFNKVTLADAGFIKQ |
| | YGDCLGDIAARDLICAQKFNGLTVLPPLLTDEMIAQYTSALLAG |
| | TITSGWTFGAGAALQIPFAMQMAYRENGIGVTQNVLYENQKLI |
| | ANQFNSAIGKIQDSLSSTASALGKLQNVVNQNAQALNTLVKQL |
| | SSNFGAISSVLNDILSRLDPPEAEVQIDRLITGRLQSLQTYVTQQL |
| | IRAAEIRASANLAATKMSECVLGQSKRVDFCGKGYHLMSFPQS |
| | APHGVVFLHVTYVPAQEKNFTTAPAICHDGKAHFPREGVFVSN |
| | GTHWFVTQRNFYEPQIITTDNTFVSGNCDVVIGIVNNTVYDPLQ |
| | PELDSFKEELDKYFKNHTSPDVDLGDISGINASVVNIQKEIDRLN |
| | EVAKNLNESLIDLQELGKYEQYIKWPWYIWLGFIAGLIAIVMVT |
| | IMLCCMTSCCSCLKGCCSCGSCCKFDEDDSEPVLKGVKLHYT |
| SEQ ID NO. 75 | MMTPENDEEQTSVFSATVYGDKIQGKNKRKRVIGLCIRISMVIS |
| Monkeypox | LLSMITMSAFLIVRLNQCMSANEAAITDSAVAVAAASSTHRKV |
| virus A35R | ASSTTQYDHKESCNGLYYQGSCYILHSDYKSFEDAKANCAAES |
| Amino Acid | STLPNKSDVLTTWLIDYVEDTWGSDGNPITKTTSDYQDSDVSQ |
| Seq. | EVRKYFCT |
| SEQ ID NO. 76 | MAAVKTPVIVVPVIDRPPSETFPNVHEHINDQKFDDVKDNEVM |
| Monkeypox | QEKRDVVIVNDDPDHYKDYVFIQWTGGNIRDDDKYTHFFSGFC |
| virus H3L | NTMCTEETKRNIARHLALWDSKFFTELENKNVEYVVIIENDNVI |
| Amino Acid | EDITFLRPVLKAIHDKKIDILQMREIITGNKVKTELVIDKDHAIFT |
| Seq. | YTGGYDVSLSAYIIRVTTALNIVDEIIKSGGLSSGFYFEIARIENE |
| | MKINRQIMDNSAKYVEHDPRLVAEHRFETMKPNFWSRIGTVA |
| | AKRYPGVMYTFTTPLISFFGLFDINVIGLIVILFIMFMLIFNVKSK |
| | LLWFLTGTFVTAFI |
| SEQ ID NO. 77 | MDHNQYLLTMFFADDDSFFKYFASQDDESSLSDILQITQYLDFL |
| Monkeypox | LLLLIQSKNKLEAVGHCYESLSEEYRQLTKFTDSQDFKKLFNKV |
| virus L1R | PIVTDGRVKLNKGYLFDFVISLMRFKKESALATTAIDPVRYIDPR |
| Amino Acid | RDIAFSNVMDILKSNKVEQ |
| Seq. | |
| SEQ ID NO. 78 | MIYMPNLPSLGSKAPDFKANTTNGPIRLSDYKGNWIVLFSHPGD |
| Clostridioides | FTPVCTTEFLCFAKYYDEFKKRNTELIGLSVDSNSSHLAWMYNI |
| difficile 630 | SLLTGVEIPFPIIEDRDMRIAKLYGMISKPMSDTSTVRSVFIIDNN |
| spore coat | QILRTILYYPLTTGRNIPEILRIVDALQTSDRDNIVTPANWFPGMP |
| protein: | VILPYPKNYKELKNRVNSCNKKYSCMDWYLCFVPDNYNDEEV |
| peroxiredoxin/ | SKKIDNTCSWKKEHTKNIENECNCEHEHHDYLNKALDCKQEH |
| chitinase Amino | KTDIKDDCNHEKKHTKNTNKVHNSKQDKFKDKSCDEMNFNY |
| Acid Seq. | DKDESCDKINSSYNKEDSSYEDFYKHNYKNYDYTSEKNTKKIA |
| | MKTLKDSKKLVRPQITDPYNPIVENANCPDINPIVAEYVLGNPT |
| | NVDAQLLDAVIFAFAEIDQSGNLFIPYPRFLNQLLALKGEKPSLK |
| | VIVAIGGWGAEGFSDAALTPTSRYNFARQVNQMINEYALDGIDI |
| | DWEYPGSSASGITSRPQDRENFTLLLTAIRDVIGDDKWLSVAGT |
| | GDRGYINSSAEIDKIAPIIDYFNLMSYDFTAGETGPNGRKHQAN |
| | LFDSDLSLPGYSVDAMVRNLENAGMPSEKILLGIPFYGRLGATI |
| | TRTYDELRRDYINKNGYEYRFDNTAQVPYLVKDGDFAMSYDD |
| | ALSIFLKTQYVLRNCLGGVFSWTSTYDQANILARTMSIGINDPE |
| | VLKEELEGIYGQF |
| SEQ ID NO. 79 | MRVNTNVSALIANNQMGRNVNGQSKSMEKLSSGVRIKRAADD |
| Clostridioides | AAGLAISEKMRAQIKGLDQAGRNVQDGISVVQTAEGSLEETGN |
| difficile 630 | ILQRMRTLSLQSANEINNTEEREKIADELTQLKDEIERISSSTEFN |
| flagellin C | GKKLLDGTSSTIRLQVGASYGTNVSGTSNNNNEIKIQLVNTASI |
| Amino Acid | MASAGITTASIGSMKAGGTTGTDAAKTMVSSLDAALKSLNSSR |
| Seq. | AKLGAQQNRLESTQNNLNNTLENVTAAESRIRDTDVASEMVN |
| | LSKMNILVQASQSMLAQANQQPQGVLQLLG |
| SEQ ID NO. 80 | GDGDYVDFLITSPGQGDKITTSKLVALKDLTGASADAIIAGTSS |
| Clostridioides | ADGVVTNTGAASGSTETNSAGTKLAMSAIFDTAYTDSSETAVK |
| difficile Surface | ITIKADMNDTKFGKAGETTYSTGLTFEDGSTEKIVKLGDSDIIDI |
| layer protein A | TKALKLTVVPGSKATVKFAEKTPSASVQPVITKLRIINAKEETID |
| (Fragment) | IDASSSKTAQDLA |
| Amino Acid | |
| Seq. | |
| SEQ ID NO. 81 | MSDEGPGTGPGNGLGEKGDTSGPEGSGGSGPQRRGGDNHGRG |
| Epstein-Barr | RGRGRGRGGGRPGAPGGSGSGPRHRDGVRRPQKRPSCIGCKGT |
| virus (strain | HGGTGAGAGAGGAGAGGAGAGGGAGAGGGAGGAGGAGGA |
| B95-8) | GAGGGAGAGGGAGGAGGAGAGGGAGAGGGAGGAGAGGGA |
| EBNA1_EBVB9 | GGAGGAGAGGGAGAGGGAGGAGAGGGAGGAGGAGAGGGA |
| Epstein-Barr | GAGGAGGAGGAGAGGAGAGGGAGGAGGAGAGGAGAGGAG |
| nuclear antigen 1 | AGGAGAGGAGGAGAGGAGGAGAGGAGGAGAGGGAGGAGA |
| Amino Acid Seq | GGGAGGAGAGGAGGAGAGGAGGAGAGGAGGAGAGGGAGA |
| | GGAGAGGGGRGRGGSGGRGRGGSGGRGRGGSGGRRGRGRER |
| | ARGGSRERARGRGRGRGEKRPRSPSSQSSSSGSPPRRPPPGRRPF |
| | FHPVGEADYFEYHQEGGPDGEPDVPPGAIEQGPADDPGEGPST |
| | GPRGQGDGGRRKKGGWFGKHRGQGGSNPKFENIAEGLRALLA |
| | RSHVERTTDEGTWVAGVFVYGGSKTSLYNLRRGTALAIPQCRL |
| | TPLSRLPFGMAPGPGPQPGPLRESIVCYFMVFLQTHIFAEVLKD |
| | AIKDLVMTKPAPTCNIRVTVCSFDDGVDLPPWFPPMVEGAAAE |
| | GDDGDDGDEGGDGDEGEEGQE |
| SEQ ID NO. 82 | MTRRRVLSVVVLLAALACRLGAQTPEQPAPPATTVQPTATRQQ |
| Epstein-Barr | TSFPFRVCELSSHGDLFRFSSDIQCPSFGTRENHTEGLLMVFKDN |
| virus (strain | IIPYSFKVRSYTKIVTNILIYNGWYADSVTNRHEEKFSVDSYETD |
| B95-8) | QMDTIYQCYNAVKMTKDGLTRVYVDRDGVNITVNLKPTGGLA |
| GB_EBVB9 | NGVRRYASQTELYDAPGWLIWTYRTRTTVNCLITDMMAKSNS |
| Envelope | PFDFFVTTTGQTVEMSPFYDGKNKETFHERADSFHVRTNYKIV |
| glycoprotein B | DYDNRGTNPQGERRAFLDKGTYTLSWKLENRTAYCPLQHWQT |
| Amino Acid | FDSTIATETGKSIHFVTDEGTSSFVTNTTVGIELPDAFKCIEEQVN |
| Seq. | KTMHEKYEAVQDRYTKGQEAITYFITSGGLLLAWLPLTPRSLA |
| | TVKNLTELTTPTSSPPSSPSPPAPSAARGSTPAAVLRRRRRDAGN |
| | ATTPVPPTAPGKSLGTLNNPATVQIQFAYDSLRRQINRMLGDLA |
| | RAWCLEQKRQNMVLRELTKINPTTVMSSIYGKAVAAKRLGDVI |
| | SVSQCVPVNQATVTLRKSMRVPGSETMCYSRPLVSFSFINDTKT |
| | YEGQLGTDNEIFLTKKMTEVCQATSQYYFQSGNEIHVYNDYHH |
| | FKTIELDGIATLQTFISLNTSLIENIDFASLELYSRDEQRASNVFD |
| | LEGIFREYNFQAQNIAGLRKDLDNAVSNGRNQFVDGLGELMDS |
| | LGSVGQSITNLVSTVGGLFSSLVSGFISFFKNPFGGMLILVLVAG |
| | VVILVISLTRRTRQMSQQPVQMLYPGIDELAQQHASGEGPGINPI |
| | SKTELQAIMLALHEQNQEQKRAAQRAAGPSVASRALQAARDR |
| | FPGLRRRRYHDPETAAALLGEAETEF |
| SEQ ID NO. 83 | MQLLCVFCLVLLWEVGAASLSEVKLHLDIEGHASHYTIPWTEL |
| Epstein-Barr | MAKVPGLSPEALWREANVTEDLASMLNRYKLIYKTSGTLGIAL |
| virus (strain | AEPVDIPAVSEGSMQVDASKVHPGVISGLNSPACMLSAPLEKQL |
| B95-8) | FYYIGTMLPNTRPHSYVFYQLRCHLSYVALSINGDKFQYTGAM |
| GH_EBVB9 | TSKFLMGTYKRVTEKGDEHVLSLVFGKTKDLPDLRGPFSYPSL |
| Envelope | TSAQSGDYSLVIVTTFVHYANFHNYFVPNLKDMFSRAVTMTAA |
| glycoprotein H | SYARYVLQKLVLLEMKGGCREPELDTETLTTMFEVSVAFFKVG |
| Amino Acid | HAVGETGNGCVDLRWLAKSFFELTVLKDIIGICYGATVKGMQS |
| Seq. | YGLERLAAMLMATVKMEELGHLTTEKQEYALRLATVGYPKA |
| | GVYSGLIGGATSVLLSAYNRHPLFQPLHTVMRETLFIGSHVVLR |
| | ELRLNVTTQGPNLALYQLLSTALCSALEIGEVLRGLALGTESGL |
| | FSPCYLSLRFDLTRDKLLSMAPQEATLDQAAVSNAVDGFLGRL |
| | SLEREDRDAWHLPAYKCVDRLDKVLMIIPLINVTFIISSDREVRG |
| | SALYEASTTYLSSSLFLSPVIMNKCSQGAVAGEPRQIPKIQNFTR |
| | TQKSCIFCGFALLSYDEKEGLETTTYITSQEVQNSILSSNYFDFD |
| | NLHVHYLLLTTNGTVMEIAGLYEERAHVVLAIILYFIAFALGIFL |
| | VHKIVMFFL |
| SEQ ID NO. 84 | MEAALLVCQYTIQSLIHLTGEDPGFFNVEIPEFPFYPTCNVCTAD |
| Epstein-Barr | VNVTINFDVGGKKHQLDLDFGQLTPHTKAVYQPRGAFGGSEN |
| virus (strain | ATNLFLLELLGAGELALTMRSKKLPINVTTGEEQQVSLESVDVY |
| B95-8) | FQDVFGTMWCHHAEMQNPVYLIPETVPYIKWDNCNSTNITAV |
| GP350_EBVB9 | VRAQGLDVTLPLSLPTSAQDSNFSVKTEMLGNEIDIECIMEDGEI |
| Envelope | SQVLPGDNKFNITCSGYESHVPSGGILTSTSPVATPIPGTGYAYS |
| glycoprotein | LRLTPRPVSRFLGNNSILYVFYSGNGPKASGGDYCIQSNIVESDE |
| GP350 Amino | IPASQDMPTNTTDITYVGDNATYSVPMVTSEDANSPNVTVTAF |
| Acid Seq. | WAWPNNTETDFKCKWTLTSGTPSGCENISGAFASNRTFDITVS |
| | GLGTAPKTLIITRTATNATTTTHKVIFSKAPESTTTSPTLNTTGFA |
| | DPNTTTGLPSSTHVPTNLTAPASTGPTVSTADVTSPTPAGTTSGA |
| | SPVTPSPSPWDNGTESKAPDMTSSTSPVTTPTPNATSPTPAVTTP |
| | TPNATSPTPAVTTPTPNATSPTLGKTSPTSAVTTPTPNATSPTLG |
| | KTSPTSAVTTPTPNATSPTLGKTSPTSAVTTPTPNATGPTVGETS |
| | PQANATNHTLGGTSPTPVVTSQPKNATSAVTTGQHNITSSSTSS |
| | MSLRPSSNPETLSPSTSDNSTSHMPLLTSAHPTGGENITQVTPASI |
| | STHHVSTSSPAPRPGTTSQASGPGNSSTSTKPGEVNVTKGTPPQ |
| | NATSPQAPSGQKTAVPTVTSTGGKANSTTGGKHTTGHGARTST |
| | EPTTDYGGDSTTPRPRYNATTYLPPSTSSKLRPRWTFTSPPVTTA |
| | QATVPVPPTSQPRFSNLSMLVLQWASLAVLTLLLLLVMADCAF |
| | RRNLSTSHTYTTPPYDDAETYV |
| SEQ. ID NO. 85 | MEHDLERGPPGPRRPPRGPPLSSSLGLALLLLLLALLFWLYIVM |
| Epstein-Barr | SDWTGGALLVLYSFALMLIIIILIIFIFRRDLLCPLGALCILLLMIT |
| virus (strain | LLLIALWNLHGQALFLGIVLFIFGCLLVLGIWIYLLEMLWRLGA |
| B95-8) Latent | TIWQLLAFFLAFFLDLILLIIALYLQQNWWTLLVDLLWLLLFLAI |
| membrane | LIWMYYHGQRHSDEHHHDDSLPHPQQATDDSGHESDSNSNEG |
| protein 1 Amino | RHHLLVSGAGDGPPLCSQNLGAPGGGPDNGPQDPDNTDDNGP |
| Acid Seq. | QDPDNTDDNGPHDPLPQDPDNTDDNGPQDPDNTDDNGPHDPL |
| | PHSPSDSAGNDGGPPQLTEEVENKGGDQGPPLMTDGGGGHSH |
| | DSGHGGGDPHLPTLLLGSSGSGGDDDDPHGPVQLSYYD |
| SEQ ID NO. 86 | MGSLEMVPMGAGPPSPGGDPDGYDGGNNSQYPSASGSSGNTP |
| Epstein-Barr | TPPNDEERESNEEPPPPYEDPYWGNGDRHSDYQPLGTQDQSLY |
| virus (strain | LGLQHDGNDGLPPPPYSPRDDSSQHIYEEAGRGSMNPVCLPVIV |
| B95-8) Latent | APYLFWLAAIAASCFTASVSTVVTATGLALSLLLLAAVASSYA |
| membrane | AAQRKLLTPVTVLTAVVTFFAICLTWRIEDPPFNSLLFALLAAA |
| protein 2 Amino | GGLQGIYVLVMLVLLILAYRRRWRRLTVCGGIMFLACVLVLIV |
| Acid Seq. | DAVLQLSPLLGAVTVVSMTLLLLAFVLWLSSPGGLGTLGAALL |
| | TLAAALALLASLILGTLNLTTMFLLMLLWTLVVLLICSSCSSCPL |
| | SKILLARLFLYALALLLLASALIAGGSILQTNFKSLSSTEFIPNLFC |
| | MLLLIVAGILFILAILTEWGSGNRTYGPVFMCLGGLLTMVAGA |
| | VWLTVMSNTLLSAWILTAGFLIFLIGFALFGVIRCCRYCCYYCL |
| | TLESEERPPTPYRNTV |
| SEQ ID NO. 87 | MNRTTFCCLSLTTALILTACSSGGGGVAADIGAGLADALTAPLD |
| Neisseria | HKDKSLQSLTLDQSVRKNEKLKLAAQGAEKTYGNGDSLNTGK |
| meningitidis | LKNDKVSRFDFIRQIEVDGQLITLESGEFQIYKQDHSAVVALQIE |
| Factor H-binding | KINNPDKIDSLINQRSFLVSGLGGEHTAFNQLPGGKAEYHGKAF |
| protein Amino | SSDDPNGRLHYSIDFTKKQGYGRIEHLKTPEQNVELASAELKAD |
| Acid Seq. | EKSHAVILGDTRYGSEEKGTYHLALFGDRAQEIAGSATVKIREK |
| | VHEIGIAGKQ |
| SEQ ID NO. 88 | MKHFPSKVLTTAILATFCSGALAATNDDDVKKAATVAIAAAYN |
| Neisseria | NGQEINGFKAGETIYDIDEDGTITKKDATAADVEADDFKGLGL |
| meningitidis | KKVVTNLTKTVNENKQNVDAKVKAAESEIEKLTTKLADTDAA |
| serogroup B | LADTDAALDATTNALNKLGENITTFAEETKTNIVKIDEKLEAVA |
| Neisseria | DTVDKHAEAFNDIADSLDETNTKADEAVKTANEAKQTAEETK |
| adhesin A | QNVDAKVKAAETAAGKAEAAAGTANTAADKAEAVAAKVTDI |
| Amino Acid | KADIATNKDNIAKKANSADVYTREESDSKFVRIDGLNATTEKL |
| Seq. | DTRLASAEKSIADHDTRLNGLDKTVSDLRKETRQGLAEQAALS |
| | GLFQPYNVGRFNVTAAVGGYKSESAVAIGTGFRFTENFAAKAG |
| | VAVGTSSGSSAAYHVGVNYEW |
| SEQ ID NO. 89 | MFERSVIAMACIFALSACGGGGGGSPDVKSADTLSKPAAPVVS |
| Neisseria | EKETEAKEDAPQAGSQGQGAPSAQGSQDMAAVSEENTGNGGA |
| meningitidis | ATADNPKNEDEGAQDDMPQNAADTDSLTPNHTPASNMPAVN |
| Neisserial | MENQAPDTGESVQPANQPDMANAADGMQGDDPSAGGQNAG |
| heparin binding | NTAAQGANQAGNNQAAGSSDPIPASNPATTNSGGDFGRTNVA |
| antigen Amino | NGIKLDGPSQNITLTHCKDTVCGSNFLAEEAPSKSEFESLDDSGR |
| Acid Seq. | INKYKKDGQDKFTNLVATEVKANGTNKYVIIYKDKSTSSARVR |
| | RSARSRRSLPAEMPLIPVNQADTLIVDGEAVSLTGHSGNIFAPEG |
| | NYRYLTYGAEKLSGGSYALRVQGEPAKGEMLAGTAVYNGEVL |
| | HFHTENGRPYPTRGRFAAKVDFGSKSVDGIIDSGDDLHMGTQK |
| | FKAAIDGNGFKGTWTENGGGDVSGRFYGPAGEEVAGKYSYRP |
| | TDAEKGGFGVFAGKKEQD |
| SEQ ID NO. 90 | MDGTLFPGDDDLAIPATEFFSTKAAKKPEAKREAIVKADEDDN |
| Vaccinia virus | EETLKQRLTNLEKKITNVTTKFEQIEKCCKRNDEVLFRLENHAE |
| (strain Western | TLRAAMISLAKKIDVQTGRRPYE |
| Reserve) Protein | |
| A27 Amino Acid | |
| Seq. | |
| SEQ ID NO. 91 | MMTPENDEEQTSVFSATVYGDKIQGKNKRKRVIGLCIRISMVIS |
| Vaccinia virus | LLSMITMSAFLIVRLNQCMSANEAAITDAAVAVAAASSTHRKV |
| (strain Western | ASSTTQYDHKESCNGLYYQGSCYILHSDYQLFSDAKANCTAES |
| Reserve) EEV | STLPNKSDVLITWLIDYVEDTWGSDGNPITKTTSDYQDSDVSQE |
| membrane | VRKYFCVKTMN |
| phosphoglycoprotein | |
| Amino Acid | |
| Seq. | |
| SEQ ID NO. 92 | MKTISVVTLLCVLPAVVYSTCTVPTMNNAKLTSTETSFNDKQK |
| Vaccinia virus | VTFTCDQGYHSSDPNAVCETDKWKYENPCKKMCTVSDYISEL |
| B5R (Fragment) | YNKPLYEVNSTMTLSCNGETKYFRCEEKNGNTSWNDTVTCPN |
| Amino Acid | AECQPLQLEHGSCQPVKEKYSFGEYMTINCDVGYEVIGASYISC |
| Seq. | TANSWNVIPSCQQKCDMPSLSNGLISGSTFSIGGVIHLSCKSGFT |
| | LTGSPSSTCIDGKWNPVLPICVRTNEEFDPVDDGPDDETDLSKL |
| | SKDVVQYEQEIESLEATYHIIIVALTIMGVIFLISVIVLVCSCDKN |
| | NDQYKFHKLLP |
| SEQ ID NO. 93 | MAAVKTPVIVVPVIDRPPSETFPNVHEHINDQKFDDVKDNEVM |
| Vaccinia virus | PEKRNVVVVKDDPDHYKDYAFIQWTGGNIRNDDKYTHFFSGF |
| Envelope protein | CNTMCTEETKRNIARHLALWDSNFFTELENKKVEYVVIVENDN |
| H3 Amino Acid | VIEDITFLRPVLKAMHDKKIDILQMREIITGNKVKTELVMDKNH |
| Seq. | AIFTYTGGYDVSLSAYIIRVTTALNIVDEIIKSGGLSSGFYFEIARI |
| | ENEMKINRQILDNAAKYVEHDPRLVAEYRFENMKPNFWSRIGT |
| | AAAKRYPGVMYAFTTPLISFFGLFDINVIGLIVILFIMFMLIFNVK |
| | SKLLWFLTGTFVTAFI |
| SEQ ID NO. 94 | MGAAASIQTTVNTLSERISSKLEQEANASAQTKCDIEIGNFYIRQ |
| Vaccinia virus | NHGCNLTVKNMCSADADAQLDAVLSAATETYSGLTPEQKAYV |
| (strain Western | PAMFTAALNIQTSVNTVVRDFENYVKQTCNSSAVVDNKLKIQN |
| Reserve) IMV | VIIDECYGAPGSPTNLEFINTGSSKGNCAIKALMQLTTKATTQIA |
| membrane | PKQVAGTGVQFYMIVIGVIILAALFMYYAKRMLFTSTNDKIKLI |
| protein Amino | LANKENVHWTTYMDTFFRTSPMVIATTDMQN |
| Acid Seq. | |
| SEQ ID NO. 95 | MAVILNKRYYSNLLILILMISECSVGILHYEKLSKIGLVKGITRK |
| Nipah virus | YKIKSNPLTKDIVIKMIPNVSNMSQCTGSVMENYKTRLNGILTPI |
| Fusion | KGALEIYKNNTHDLVGDVRLAGVIMAGVAIGIATAAQITAGVA |
| glycoprotein F0 | LYEAMKNADNINKLKSSIESTNEAVVKLQETAEKTVYVLTALQ |
| Amino Acid | DYINTNLVPTIDKISCKQTELSLDLALSKYLSDLLFVFGPNLQDP |
| Seq. | VSNSMTIQAISQAFGGNYETLLRTLGYATEDFDDLLESDSITGQI |
| | IYVDLSGYYIIVRVYFPILTEIQQAYIQELLPVSFNNDNSEWISIVP |
| | NFILVRNTLISNIEIGFCLITKRSVICNQDYATPMTNNMRECLTGS |
| | TEKCPRELVVSSHVPRFALSNGVLFANCISVTCQCQTTGRAISQS |
| | GEQTLLMIDNTTCPTAVLGNVIISLGKYLGSVNYNSEGIAIGPPV |
| | FTDKVDISSQISSMNQSLQQSKDYIKEAQRLLDTVNPSLISMLSM |
| | IILYVLSIASLCIGLITFISFIIVEKKRNTYSRLEDRRVRPTSSGDLY |
| | YIGT |
| SEQ ID NO. 96 | MPAENKKVRFENTTSDKGKIPSKVIKSYYGTMDIKKINEGLLDS |
| Nipah virus | KILSAFNTVIALLGSIVIIVMNIMIIQNYTRSTDNQAVIKDALQGI |
| Glycoprotein G | QQQIKGLADKIGTEIGPKVSLIDTSSTITIPANIGLLGSKISQSTASI |
| Amino Acid | NENVNEKCKFTLPPLKIHECNISCPNPLPFREYRPQTEGVSNLVG |
| Seq. | LPNNICLQKTSNQILKPKLISYTLPVVGQSGTCITDPLLAMDEGY |
| | FAYSHLERIGSCSRGVSKQRIIGVGEVLDRGDEVPSLFMTNVWT |
| | PPNPNTVYHCSAVYNNEFYYVLCAVSTVGDPILNSTYWSGSLM |
| | MTRLAVKPKSNGGGYNQHQLALRSIEKGRYDKVMPYGPSGIK |
| | QGDTLYFPAVGFLVRTEFKYNDSNCPITKCQYSKPENCRLSMGI |
| | RPNSHYILRSGLLKYNLSDGENPKVVFIEISDQRLSIGSPSKIYDS |
| | LGQPVFYQASFSWDTMIKFGDVLTVNPLVVNWRNNTVISRPGQ |
| | SQCPRFNTCPEICWEGVYNDAFLIDRINWISAGVFLDSNQTAEN |
| | PVFTVFKDNEILYRAQLASEDTNAQKTITNCFLLKNKIWCISLVE |
| | IYDTGDNVIRPKLFAVKIPEQCT |
| SEQ ID NO. 97 | MGTVNKPVVGVLMGFGIITGTLRITNPVRASVLRYDDFHTDED |
| Varicella-zoster | KLDTNSVYEPYYHSDHAESSWVNRGESSRKAYDHNSPYIWPR |
| virus (strain | NDYDGFLENAHEHHGVYNQGRGIDSGERLMQPTQMSAQEDLG |
| Dumas) | DDTGIHVIPTLNGDDRHKIVNVDQRQYGDVFKGDLNPKPQGQR |
| Envelope | LIEVSVEENHPFTLRAPIQRIYGVRYTETWSFLPSLTCTGDAAPA |
| glycoprotein E | IQHICLKHTTCFQDVVVDVDCAENTKEDQLAEISYRFQGKKEA |
| Amino Acid | DQPWIVVNTSTLFDELELDPPEIEPGVLKVLRTEKQYLGVYIWN |
| Seq | MRGSDGTSTYATFLVTWKGDEKTRNPTPAVTPQPRGAEFHMW |
| | NYHSHVFSVGDTFSLAMHLQYKIHEAPFDLLLEWLYVPIDPTC |
| | QPMRLYSTCLYHPNAPQCLSHMNSGCTFTSPHLAQRVASTVYQ |
| | NCEHADNYTAYCLGISHMEPSFGLILHDGGTTLKFVDTPESLSG |
| | LYVFVVYFNGHVEAVAYTVVSTVDHFVNAIEERGFPPTAGQPP |
| | ATTKPKEITPVNPGTSPLLRYAAWTGGLAAVVLLCLVIFLICTA |
| | KRMRVKAYRVDKSPYNQSMYYAGLPVDDFEDSESTDTEEEFG |
| | NAIGGSHGGSSYTVYIDKTR |
| SEQ ID NO. 98 | METLCQRLNVCQDKILTHYENDSTDLRDHIDYWKHMRLECAI |
| Human | YYKAREMGFKHINHQVVPTLAVSKNKALQAIELQLTLETIYNS |
| papillomavirus | QYSNEKWTLQDVSLEVYLTAPTGCIKKHGYTVEVQFDGDICNT |
| type 16 | MHYTNWTHIYICEEASVTVVEGQVDYYGLYYVHEGIRTYFVQF |
| Regulatory | KDDAEKYSKNKVWEVHAGGQVILCPTSVFSSNEVSSPEIIRQHL |
| protein E2 | ANHPAATHTKAVALGTEETQTTIQRPRSEPDTGNPCHTTKLLHR |
| Amino Acid | DSVDSAPILTAFNSSHKGRINCNSNTTPIVHLKGDANTLKCLRY |
| Seq. | RFKKHCTLYTAVSSTWHWTGHNVKHKSAIVTLTYDSEWQRDQ |
| | FLSQVKIPKTITVSTGFMSI |
| SEQ ID NO. 99 | MHQKRTAMFQDPQERPRKLPQLCTELQTTIHDIILECVYCKQQL |
| Human | LRREVYDFAFRDLCIVYRDGNPYAVCDKCLKFYSKISEYRHYC |
| papillomavirus | YSLYGTTLEQQYNKPLCDLLIRCINCQKPLCPEEKQRHLDKKQR |
| type 16 Protein | FHNIRGRWTGRCMSCCRSSRTRRETQL |
| E6 Amino Acid | |
| Seq. | |
| SEQ ID NO. 100 | MHGDTPTLHEYMLDLQPETTDLYCYEQLNDSSEEEDEIDGPAG |
| Human | QAEPDRAHYNIVTFCCKCDSTLRLCVQSTHVDIRTLEDLLMGTL |
| papillomavirus | GIVCPICSQKP |
| type 16 Protein | |
| E7 Amino Acid | |
| Seq. | |
| SEQ ID NO. 101 | MQTPKETLSERLSCVQDKIIDHYENDSKDIDSQIQYWQLIRWEN |
| Human | AIFFAAREHGIQTLNHQVVPAYNISKSKAHKAIELQMALQGLA |
| papillomavirus | QSAYKTEDWTLQDTCEELWNTEPTHCFKKGGQT |
| type 18 | VQVYFDGNKDNCMTYVAWDSVYYMTDAGTWDKTATCVSHR |
| Regulatory | GLYYVKEGYNTFYIEFKSECEKYGNTGTWEVHFGNNVIDCNDS |
| protein E2 | MCSTSDDTVSATQLVKQLQHTPSPYSSTVSVGTAKTYGQTSAA |
| Amino Acid | TRPGHCGLAEKQHCGPVNPLLGAATPTGNNKRRKLCSGNTTPII |
| Seq. | HLKGDRNSLKCLRYRLRKHSDHYRDISSTWHWTGAGNEKTGI |
| | LTVTYHSETQRTKFLNTVAIPDSVQILVGYMTM |
| SEQ ID NO. 102 | MARFEDPTRRPYKLPDLCTELNTSLQDIEITCVYCKTVLELTEVF |
| Human | EFAFKDLFVVYRDSIPHAACHKCIDFYSRIRELRHYSDSVYGDT |
| papillomavirus | LEKLTNTGLYNLLIRCLRCQKPLNPAEKLRHLNEKRRFHNIAGH |
| type 18 Protein | YRGQCHSCCNRARQERLQRRRETQV |
| E6 Amino Acid | |
| Seq. | |
| SEQ ID NO. 103 | MHGPKATLQDIVLHLEPQNEIPVDLLCHEQLSDSEEENDEIDGV |
| Human | NHQHLPARRAEPQRHTMLCMCCKCEARIKLVVESSADDLRAF |
| papillomavirus | QQLFLNTLSFVCPWCASQQ |
| type 18 Protein | |
| E7 Amino Acid | |
| Seq. | |
| SEQ ID NO. 104 | MEAIAKRLDACQEQLLELYEENSTDLNKHVLHWKCMRHESVL |
| Human | LYKAKQMGLSHIGMQVVPPLKVSEAKGHNAIEMQMHLESLLK |
| papillomavirus | TEYSMEPWTLQETSYEMWQTPPKRCFKKRGKTVEVKFDGCAN |
| type 6a | NTMDYVVWTDVYVQDTDSWVKVHSMVDAKGIYYTCGQFKT |
| Regulatory | YYVNFVKEAEKYGSTKQWEVCYGSTVICSPASVSSTTQEVSIPE |
| protein E2 | STTYTPAQTSTPVSSSTQEDAVQTPPRKRARGVQQSPCNALCVA |
| Amino Acid | HIGPVDSGNHNLITNNHDQHQRRNNSNSSATPIVQFQGESNCLK |
| Seq. | CFRYRLNDKHRHLFDLISSTWHWASPKAPHKHAIVTVTYHSEE |
| | QRQQFLNVVKIPPTIRHKLGFMSLHLL |
| SEQ ID NO. 105 | MESANASTSATTIDQLCKTFNLSMHTLQINCVFCKNALTTAEIY |
| Human | SYAYKQLKVLFRGGYPYAACACCLEFHGKINQYRHFDYAGYA |
| papillomavirus | TTVEEETKQDILDVLIRCYLCHKPLCEVEKVKHILTKARFIKLNC |
| type 6a Protein | TWKGRCLHCWTTCMEDMLP |
| E6 Amino Acid | |
| Seq. | |
| SEQ ID NO. 106 | MHGRHVTLKDIVLDLQPPDPVGLHCYEQLVDSSEDEVDEVDG |
| Human | QDSQPLKQHFQIVTCCCGCDSNVRLVVQCTETDIREVQQLLLG |
| papillomavirus | TLDIVCPICAPKT |
| type 6a Protein | |
| E7 Amino Acid | |
| Seq. | |
| SEQ ID NO. 107 | MEAIAKRLDACQDQLLELYEENSIDIHKHIMHWKCIRLESVLLH |
| Human | KAKQMGLSHIGLQVVPPLTVSETKGHNAIEMQMHLESLAKTQ |
| papillomavirus | YGVEPWTLQDTSYEMWLTPPKRCFKKQGNTVEVKFDGCEDNV |
| 11 Regulatory | MEYVVWTHIYLQDNDSWVKVTSSVDAKGIYYTCGQFKTYYV |
| protein E2 | NFNKEAQKYGSTNHWEVCYGSTVICSPASVSSTVREVSIAEPTT |
| Amino Acid | YTPAQTTAPTVSACTTEDGVSAPPRKRARGPSTNNTLCVANIRS |
| Seq. | VDSTINNIVTDNYNKHQRRNNCHSAATPIVQLQGDSNCLKCFR |
| | YRLNDKYKHLFELASSTWHWASPEAPHKNAIVTLTYSSEEQRQ |
| | QFLNSVKIPPTIRHKVGFMSLHLL |
| SEQ ID NO. 108 | MESKDASTSATSIDQLCKTFNLSLHTLQIQCVFCRNALTTAEIYA |
| Human | YAYKNLKVVWRDNFPFAACACCLELQGKINQYRHFNYAAYAP |
| papillomavirus | TVEEETNEDILKVLIRCYLCHKPLCEIEKLKHILGKARFIKLNNQ |
| 11 Protein E6 | WKGRCLHCWTTCMEDLLP |
| Amino Acid | |
| Seq. | |
| SEQ ID NO. 109 | MHGRLVTLKDIVLDLQPPDPVGLHCYEQLEDSSEDEVDKVDKQ |
| Human | DAQPLTQHYQILTCCCGCDSNVRLVVECTDGDIRQLQDLLLGT |
| papillomavirus | LNIVCPICAPKP |
| 11 Protein E7 | |
| Amino Acid | |
| Seq. | |
| SEQ ID NO. 110 | MSTNPKPQKKNKRNTNRRPQDVKFPGGGQIVGGVYLLPRRGP |
| Hepatitis C virus | RLGVRATRKTSERSQPRGRRQPIPKARRPEGRTWAQPGYPWPL |
| genotype 1a | YGNEGCGWAGWLLSPRGSRPSWGPTDPRRRSRNLGKVIDTLTC |
| Genome | GFADLMGYIPLVGAPLGGAARALAHGVRVLEDGVNYATGNLP |
| polyprotein | GCSFSIFLLALLSCLTVPASAYQVRNSTGLYHVTNDCPNSSIVYE |
| Amino Acid | AADAILHTPGCVPCVREGNASRCWVAMTPTVATRDGKLPATQ |
| Seq | LRRHIDLLVGSATLCSALYVGDLCGSVFLVGQLFTFSPRRHWT |
| | TQGCNCSIYPGHITGHRMAWDMMMNWSPTTALVMAQLLRIPQ |
| | AILDMIAGAHWGVLAGIAYFSMVGNWAKVLVVLLLFAGVDAE |
| | THVTGGSAGHTVSGFVSLLAPGAKQNVQLININGSWHLNSTAL |
| | NCNDSLNTGWLAGLFYHHKFNSSGCPERLASCRPLTDFDQGW |
| | GPISYANGSGPDQRPYCWHYPPKPCGIVPAKSVCGPVYCFTPSP |
| | VVVGTTDRSGAPTYSWGENDTDVFVLNNTRPPLGNWFGCTW |
| | MNSTGFTKVCGAPPCVIGGAGNNTLHCPTDCFRKHPDATYSRC |
| | GSGPWITPRCLVDYPYRLWHYPCTINYTIFKIRMYVGGVEHRLE |
| | AACNWTRGERCDLEDRDRSELSPLLLTTTQWQVLPCSFTTLPA |
| | LSTGLIHLHQNIVDVQYLYGVGSSIASWAIKWEYVVLLFLLLAD |
| | ARVCSCLWMMLLISQAEAALENLVILNAASLAGTHGLVSFLVF |
| | FCFAWYLKGKWVPGAVYTFYGMWPLLLLLLALPQRAYALDT |
| | EVAASCGGVVLVGLMALTLSPYYKRYISWCLWWLQYFLTRVE |
| | AQLHVWIPPLNVRGGRDAVILLMCAVHPTLVFDITKLLLAVFG |
| | PLWILQASLLKVPYFVRVQGLLRFCALARKMIGGHYVQMVIIK |
| | LGALTGTYVYNHLTPLRDWAHNGLRDLAVAVEPVVFSQMETK |
| | LITWGADTAACGDIINGLPVSARRGREILLGPADGMVSKGWRL |
| | LAPITAYAQQTRGLLGCIITSLTGRDKNQVEGEVQIVSTAAQTFL |
| | ATCINGVCWTVYHGAGTRTIASPKGPVIQMYTNVDQDLVGWP |
| | APQGSRSLTPCTCGSSDLYLVTRHADVIPVRRRGDSRGSLLSPR |
| | PISYLKGSSGGPLLCPAGHAVGIFRAAVCTRGVAKAVDFIPVEN |
| | LETTMRSPVFTDNSSPPVVPQSFQVAHLHAPTGSGKSTKVPAAY |
| | AAQGYKVLVLNPSVAATLGFGAYMSKAHGIDPNIRTGVRTITT |
| | GSPITYSTYGKFLADGGCSGGAYDIIICDECHSTDATSILGIGTVL |
| | DQAETAGARLVVLATATPPGSVTVPHPNIEEVALSTTGEIPFYG |
| | KAIPLEVIKGGRHLIFCHSKKKCDELAAKLVALGINAVAYYRGL |
| | DVSVIPTSGDVVVVATDALMTGYTGDFDSVIDCNTCVTQTVDF |
| | SLDPTFTIETITLPQDAVSRTQRRGRTGRGKPGIYRFVAPGERPS |
| | GMFDSSVLCECYDAGCAWYELTPAETTVRLRAYMNTPGLPVC |
| | QDHLEFWEGVFTGLTHIDAHFLSQTKQSGENLPYLVAYQATVC |
| | ARAQAPPPSWDQMWKCLIRLKPTLHGPTPLLYRLGAVQNEITL |
| | THPVTKYIMTCMSADLEVVTSTWVLVGGVLAALAAYCLSTGC |
| | VVIVGRVVLSGKPAIIPDREVLYREFDEMEECSQHLPYIEQGMM |
| | LAEQFKQKALGLLQTASRQAEVIAPAVQTNWQKLETFWAKHM |
| | WNFISGIQYLAGLSTLPGNPAIASLMAFTAAVTSPLTTSQTLLEN |
| | ILGGWVAAQLAAPGAATAFVGAGLAGAAIGSVGLGKVLIDILA |
| | GYGAGVAGALVAFKIMSGEVPSTEDLVNLLPAILSPGALVVGV |
| | VCAAILRRHVGPGEGAVQWMNRLIAFASRGNHVSPTHYVPESD |
| | AAARVTAILSSLTVTQLLRRLHQWISSECTTPCSGSWLRDIWDW |
| | ICEVLSDFKTWLKAKLMPQLPGIPFVSCQRGYKGVWRVDGIMH |
| | TRCHCGAEITGHVKNGTMRIVGPRTCRNMWSGTFPINAYTTGP |
| | CTPLPAPNYTFALWRVSAEEYVEIRQVGDFHYVTGMTTDNLKC |
| | PCQVPSPEFFTELDGVRLHRFAPPCKPLLREEVSFRVGLHEYPV |
| | GSQLPCEPEPDVAVLTSMLTDPSHITAEAAGRRLARGSPPSVAS |
| | SSASQLSAPSLKATCTANHDSPDAELIEANLLWRQEMGGNITRV |
| | ESENKVVILDSFDPLVAEEDEREISVPAEILRKSRRFAQALPVWA |
| | RPDYNPPLVETWKKPDYEPPVVHGCPLPPPKSPPVPPPRKKRTV |
| | VLTESTLSTALAELATRSFGSSSTSGITGDNTTTSSEPAPSGCPPD |
| | SDAESYSSMPPLEGEPGDPDLSDGSWSTVSSEANAEDVVCCSM |
| | SYSWTGALVTPCAAEEQKLPINALSNSLLRHHNLVYSTTSRSAC |
| | QRQKKVTFDRLQVLDSHYQDVLKEVKAAASKVKANLLSVEEA |
| | CSLTPPHSAKSKFGYGAKDVRCHARKAVTHINSVWKDLLEDN |
| | VTPIDTTIMAKNEVFCVQPEKGGRKPARLIVFPDLGVRVCEKM |
| | ALYDVVTKLPLAVMGSSYGFQYSPGQRVEFLVQAWKSKKTPM |
| | GFSYDTRCFDSTVTESDIRTEEAIYQCCDLDPQARVAIKSLTERL |
| | YVGGPLTNSRGENCGYRRCRASGVLTTSCGNTLTCYIKARAAC |
| | RAAGLQDCTMLVCGDDLVVICESAGVQEDAASLRAFTEAMTR |
| | YSAPPGDPPQPEYDLELITSCSSNVSVAHDGAGKRVYYLTRDPT |
| | TPLARAAWETARHTPVNSWLGNIIMFAPTLWARMILMTHFFSV |
| | LIARDQLEQALDCEIYGACYSIEPLDLPPIIQRLHGLSAFSLHSYS |
| | PGEINRVAACLRKLGVPPLRAWRHRARSVRARLLARGGRAAI |
| | CGKYLFNWAVRTKLKLTPIAAAGQLDLSGWFTAGYSGGDIYH |
| | SVSHARPRWIWFCLLLLAAGVGIYLLPNR |
| SEQ ID NO. 111 | MSTNPKPQRKTKRNTNRRPQDVKFPGGGQIVGGVYLLPRRGPR |
| Hepatitis C virus | LGVRATRKTSERSQPRGRRQPIPKARQPEGRAWAQPGYPWPLY |
| genotype 1b | GNEGMGWAGWLLSPRGSRPSWGPTDPRRRSRNLGKVIDTLTC |
| Genome | GCSFSIFLLALLSCLTIPASAYEVRNVSGIYHVTNDCSNSSIVYEA |
| polyprotein | GFADLMGYIPLVGAPLGGVARALAHGVRVVEDGVNYATGNLP |
| Amino Acid | ADMIMHTPGCVPCVREGNSSRCWVALTPTLAARNASVPTTAIR |
| Seq. | RHVDLLVGAAAFCSAMYVGDLCGSVFLVSQLFTFSPRRHET |
| | IQDCNCSIYPGHVSGHRMAWDMMMNWSPTTALVVSQLLRIPQ |
| | AIVDMVAGAHWGVLAGLAYYSMVGNWAKVLIVMLLFAGVD |
| | LNCNDSLNTGFLAALFYTHKFNASGCPERLASCRPIDKFDQGW |
| | GETRVTGGQIARNAYSLTTLFSSGSAQNIQLININGSWHINRTA |
| | GPITYAEQGGQDQRPYCWHYAPKPCGIVSASKVCGPVYCFTPS |
| | PVVVGTTDRFGVPTYSWGENETDVLLLNNTRPPQGNWFGCTW |
| | MNGTGFTKTCGGPPCNIGGGGNNTLTCPTDCFRKHPAATYTKC |
| | GSGPWLTPRCLVDYPYRLWHYPCTANFTIFKVRMYVGGVEHR |
| | LDAACNWTRGERCNLEDRDRLELSPLLLSTTEWQVLPCSFTTLP |
| | ALSTGLIHLHQNIVDVQYLYGIGSAVVSFAIKWDYIVILFLLLAD |
| | ARVCACLWMMLLIAQAEAALENLVVLNAASVAGAHGILSFLV |
| | FFCAAWYIKGKLVPGAAYAFYGVWPLLLLLLALPPRAYAMER |
| | EMAASCGGAVFVGLVLLTLSPYYKEFLARLIWWLQYFITRAEA |
| | HLQVWIPPLNIRGGRDAIILLACVVHPELIFDITKLLLAILGPLM |
| | VLQASITQVPYFVRAQGLIRACMLVRKVAGGHYVQMAFVKLT |
| | ALTGTYVYDHLTPLRDWAHAGLRDLAVAVEPVVFSDMETKVI |
| | TWGADTAACGDIILGLPVSARRGREILLGPADSLEGQGWRLLA |
| | PITAYSQQTRGLLGCIITSLTGRDKNQVEGEVQVVSTATQSFLA |
| | TCVNGACWTVFHGAGSKTLAGPKGPITQMYTNVDLDLVGWQ |
| | APPGSRSLTPCTCGSSDLYLVTRHADVIPVRRRGDSRGSLLSPRP |
| | VSYLKGSSGGPLLCPSRHAVGIFRAAVCTRGVAKAVDFIPVES |
| | METTMRSPVFTDNSSPPAVPQTFQVAHLHAPTGSGKSTKVPAA |
| | YAAQGYKVLVLNPSVAATLGFGAYMSKAHGIDPNIRTGVRAIT |
| | TGAPITYSTYGKFLADGGCSGGAYDIIICDECHSTDSTTILGIGTV |
| | LDQAETAGARLVVLATATPPGSVTVPHPNIEEVALSNAGEIPFY |
| | GKAIPIEVIKGGRHLIFCHSKKKYDELAAKLSALGLNAVAYYRG |
| | LDVSVIPTNGDVVVVATDALMTGFTGDFDSVIDCNTCVTQTVD |
| | FSLDPTFTIETTTVPQDAVARSQRRGRTGRGRRGIYRFVTPGERP |
| | SGMFDSSVLCECYDAGCAWYELTPAETSVRLRAYLNTPGLPVC |
| | QDHLEFWESVSTGLTHIDAHFLSQTKQAGDNFPYLVAYQATVC |
| | ARAQAPPPSWDQMWKCLIRLKPTLHGPTPLLYRLGAVQNEITL |
| | THPMTKFIMACMSADLEVVTSTWVLVGGVLAALAAYCLTTGS |
| | VVIVGRIILSGRPAVIPDREVLYREFDEMEECASHLPYIEQGMQL |
| | AEQFKQKALGLLQTATKQAEAAAPVVESKWRALETFWAKHM |
| | WNFISGIQYLAGLSTLPGNPAIASLMAFTASITSPLSTQNTLLFNI |
| | WGGWVAAQLAPPSAASAFVGAGIAGAAVGSIGLGKVLVDILA |
| | GYGAGVAGALVAFKIMSGEVPSTEDLVNLLPAILSPGALVVGV |
| | VCAAILRRHVGPGEGAVQWMNRLIAFASRGNHVSPAHYVPES |
| | DAAARVTQILSGLTITQLLKRLHHWINEDCSTPCSGSWLRDV |
| | WDWICTVLTDFKTWLQSKLLPRLPGVPFFSCQRGYKGVWRGD |
| | GIMQTTCPCGAQITGHVKNGSMRIVGPKTCSSTWHGTFPINAYT |
| | TGPCAPSPAPNYSRALWRVAAEEYVEVTRVGDFHYVTGMTTD |
| | NVKCPCQVPAPEFFTEVDGVRLHRYAPACKPLLREEVTFQVGL |
| | NQYLVGSQLPCEPEPDVAVLTSMLTDPSHITAETAKRRLARGSP |
| | PSLASSSASQLSAPSLKATCTTHHDSPDVDLIEANLLWRQEMGG |
| | NITRVESENKVVILDSFDPLRAEEDEREPSVAAEILRKTKRFPPA |
| | MPIWARPDYNPPLLESWKDPDYVPPVVHGCPLPPTKAPPIPPPR |
| | RKRTVVLTESTVSSALAELATKTFGSSGSSAVDSGTATAPPDQA |
| | SDDGDQGSDVESYSSMPPLEGEPGDPDLSDGSWSTVSEEAGED |
| | VICCSMSYTWTGALITPCAAEESKLPINPLSNSLLRHHNMVYAT |
| | TSRSAGLRQKKVTFDRLQVLDDHYRDVLKEMKAKASTVKAKL |
| | LSIEEACKLTPPHSARSKFGYGAKDVRNLSSKAVNHIRSVWKD |
| | LLEDTETPIDTTVMAKSEVFCVQPEKGGRKPARLIVFPDLGVRV |
| | CEKMALYDVVSTLPQAVMGSSYGFQYSPGQRVEFLVNAWKSK |
| | KCPMGFSYDTRCFDSTVTESDIRVEESIYQCCDLAPEARQAIKSL |
| | TERLYIGGPLTNSKGQNCGYRRCRASGVLTTSCGNTLTCYLKA |
| | SAACRAAKLRDCTMLVNGDDLVVICESAGTQEDEANLRVFTE |
| | AMTRYSAPPGDPPRPEYDLELITSCSSNVSVAHDASGKRVYYLT |
| | RDPSTPLARAAWETARHTPVNSWLGNIIMYAPTLWARMILMT |
| | HFFSILLAQEQLEKALDCQIYGACYSIEPLDLPQIIERLHGLSAFS |
| | LHSYSPGEINRVASCLRKLGVPPLRVWRHRARSVRAKLLSQGG |
| | RAATCGKYLFNWAVRTKLKLTPIPAASQLDLSSWFVAGYSGG |
| | DIYHSLSRARPRWFMLCLLLLSVGVGIYLLPNR |
| SEQ ID NO. 112 | MSTNPKPQRKTKRNTNRRPQDVKFPGGGQIVGGVYLLPRRGPR |
| Hepatitis C virus | LGVRATRKTSERSQPRGRRQPIPKDRRSTGKSWGKPGYPWPLY |
| genotype 2a | GNEGLGWAGWLLSPRGSRPSWGPNDPRHRSRNVGKVIDTLTC |
| Genome | GFADLMGYIPVVGAPLGGVARALAHGVRVLEDGVNFATGNLP |
| polyprotein | GCSFSIFLLALLSCITTPVSAAEVKNISTGYMVTNDCTNDSITWQ |
| Amino Acid | LQAAVLHVPGCVPCEKVGNTSRCWIPVSPNVAVQQPGALTQG |
| Seq. | LRTHIDMVVMSATLCSALYVGDLCGGVMLAAQMFIVSPQHHW |
| | FVQDCNCSIYPGTITGHRMAWDMMMNWSPTATMILAYAMRV |
| | PEVIIDIIGGAHWGVMFGLAYFSMQGAWAKVVVILLLAAGVDA |
| | QTHTVGGSTAHNARTLTGMFSLGARQKIQLININGSWHINRTA |
| | LNCNDSLHTGFLASLFYTHSFNSSGCPERMSACRSIEAFRVGWG |
| | ALQYEDNVTNPEDMRPYCWHYPPRQCGVVSASSVCGPVYCFT |
| | PSPVVVGTTDRLGAPTYTWGENETDVFLLNSTRPPQGSWFGCT |
| | WMNSTGYTKTCGAPPCRIRADFNASMDLLCPTDCFRKHPDTTY |
| | IKCGSGPWLTPRCLIDYPYRLWHYPCTVNYTIFKIRMYVGGVE |
| | HRLTAACNFTRGDRCNLEDRDRSQLSPLLHSTTEWAILPCTYSD |
| | LPALSTGLLHLHQNIVDVQFMYGLSPALTKYIVRWEWVVLLFL |
| | LLADARVCACLWMLILLGQAEAALEKLVVLHAASAASCNGFL |
| | YFVIFFVAAWYIKGRVVPLATYSLTGLWSFGLLLLALPQQAYA |
| | YDASVHGQIGAALLVLITLFTLTPGYKTLLSRFLWWLCYLLTLA |
| | EAMVQEWAPPMQVRGGRDGIIWAVAIFCPGVVFDITKWLLAV |
| | LGPAYLLKGALTRVPYFVRAHALLRMCTMVRHLAGGRYVQM |
| | VLLALGRWTGTYIYDHLTPMSDWAANGLRDLAVAVEPIIFSPM |
| | EKKVIVWGAETAACGDILHGLPVSARLGREVLLGPADGYTSKG |
| | WSLLAPITAYAQQTRGLLGTIVVSMTGRDKTEQAGEIQVLSTVT |
| | QSFLGTTISGVLWTVYHGAGNKTLAGSRGPVTQMYSSAEGDL |
| | VGWPSPPGTKSLEPCTCGAVDLYLVTRNADVIPARRRGDKRGA |
| | LLSPRPLSTLKGSSGGPVLCPRGHAVGVFRAAVCSRGVAKSIDF |
| | IPVETLDIVTRSPTFSDNSTPPAVPQTYQVGYLHAPTGSGKSTKV |
| | PVAYAAQGYKVLVLNPSVAATLGFGAYLSKAHGINPNIRTGVR |
| | TVTTGAPITYSTYGKFLADGGCAGGAYDIIICDECHAVDSTTILG |
| | IGTVLDQAETAGVRLTVLATATPPGSVTTPHPNIEEVALGQEGEI |
| | PFYGRAIPLSYIKGGRHLIFCHSKKKCDELAAALRGMGLNAVA |
| | YYRGLDVSVIPTQGDVVVVATDALMTGFTGDFDSVIDCNVAV |
| | TQVVDFSLDPTFTITTQTVPQDAVSRSQRRGRTGRGRLGIYRYV |
| | STGERASGMFDSVVLCECYDAGAAWYELTPAETTVRLRAYFN |
| | TPGLPVCQDHLEFWEAVFTGLTHIDAHFLSQTKQSGENFAYLT |
| | AYQATVCARAKAPPPSWDVMWKCLTRLKPTLVGPTPLLYRLG |
| | SVTNEVTLTHPVTKYIATCMQADLEVMTSTWVLAGGVLAAVA |
| | AYCLATGCVCIIGRLHVNQRAVVAPDKEVLYEAFDEMEECASR |
| | AALIEEGQRIAEMLKSKIQGLLQQASKQAQDIQPAVQASWPKV |
| | EQFWAKHMWNFISGIQYLAGLSTLPGNPAVASMMAFSAALTSP |
| | LSTSTTILLNILGGWLASQIAPPAGATGFVVSGLVGAAVGSIGLG |
| | KVLVDILAGYGAGISGALVAFKIMSGEKPSMEDVVNLLPGILSP |
| | GALVVGVICAAILRRHVGPGEGAVQWMNRLIAFASRGNHVAP |
| | THYVTESDASQRVTQLLGSLTITSLLRRLHNWITEDCPIPCSGSW |
| | LRDVWDWVCTILTDFKNWLTSKLFPKMPGLPFISCQKGYKGV |
| | WAGTGIMTTRCPCGANISGNVRLGSMRITGPKTCMNIWQGTFPI |
| | NCYTEGQCVPKPAPNFKIAIWRVAASEYAEVTQHGSYHYITGL |
| | TTDNLKVPCQLPSPEFFSWVDGVQIHRFAPIPKPFFRDEVSFCVG |
| | LNSFVVGSQLPCDPEPDTDVLTSMLTDPSHITAETAARRLARGS |
| | PPSEASSSASQLSAPSLRATCTTHGKAYDVDMVDANLFMGGDV |
| | TRIESESKVVVLDSLDPMVEERSDLEPSIPSEYMLPKKRFPPALP |
| | AWARPDYNPPLVESWKRPDYQPATVAGCALPPPKKTPTPPPRR |
| | RRTVGLSESSIADALQQLAIKSFGQPPPSGDSGLSTGADAADSGS |
| | RTPPDELALSETGSISSMPPLEGEPGDPDLEPEQVELQPPPQGGV |
| | VTPGSGSGSWSTCSEEDDSVVCCSMSYSWTGALITPCSPEEEKL |
| | PINPLSNSLLRYHNKVYCTTSKSASLRAKKVTFDRMQALDAHY |
| | DSVLKDIKLAASKVTARLLTLEEACQLTPPHSARSKYGFGAKE |
| | VRSLSGRAVNHIKSVWKDLLEDTQTPIPTTIMAKNEVFCVDPTK |
| | GGKKAARLIVYPDLGVRVCEKMALYDITQKLPQAVMGASYGF |
| | QYSPAQRVEFLLKAWAEKKDPMGFSYDTRCFDSTVTERDIRTE |
| | ESIYRACSLPEEAHTAIHSLTERLYVGGPMFNSKGQTCGYRRCR |
| | ASGVLTTSMGNTITCYVKALAACKAAGIIAPTMLVCGDDLVVI |
| | SESQGTEEDERNLRAFTEAMTRYSAPPGDPPRPEYDLELITSCSS |
| | NVSVALGPQGRRRYYLTRDPTTPIARAAWETVRHSPVNSWLG |
| | NIIQYAPTIWARMVLMTHFFSILMAQDTLDQNLNFEMYGAVYS |
| | VSPLDLPAIIERLHGLDAFSLHTYTPHELTRVASALRKLGAPPLR |
| | AWKSRARAVRASLISRGGRAAVCGRYLFNWAVKTKLKLTPLP |
| | EARLLDLSSWFTVGAGGGDIYHSVSRARPRLLLLGLLLLFVGV |
| | GLFLLPAR |
| SEQ ID NO. 113 | MSTLPKPQRKTKRNTIRRPQDVKFPGGGQIVGGVYVLPRRGPR |
| Hepatitis C virus | LGVRATRKTSERSQPRGRRQPIPKARRSEGRSWAQPGYPWPLY |
| genotype 3a | GNEGCGWAGWLLSPRGSRPSWGPNDPRRRSRNLGKVIDTLTC |
| Genome | GFADLMGYIPLVGAPVGGVARALAHGVRALEDGINFATGNLP |
| polyprotein | GCSFSIFLLALFSCLIHPAASLEWRNTSGLYVLTNDCSNSSIVYE |
| Amino Acid | ADDVILHTPGCVPCVQDGNTSTCWTPVTPTVAVRYVGATTASI |
| Seq. | VQTCNCSLYPGHLSGHRMAWDMMMNWSPAVGMVVAHVLRL |
| | RSHVDLLVGAATMCSALYVGDMCGAVFLVGQAFTFRPRRHQT |
| | PQTLFDIMAGAHWGILAGLAYYSMQGNWAKVAIIMVMFSGVD |
| | AHTYTTGGTASRHTQAFAGLFDIGPQQKLQLVNTNGSWHINST |
| | ALNCNESINTGFIAGLFYYHKFNSTGCPQRLSSCKPITFFRQGWG |
| | PLTDANITGPSDDRPYCWHYAPRPCDIVPASSVCGPVYCFTPSP |
| | VVVGTTDARGVPTYTWGENEKDVFLLKSQRPPSGRWFGCSW |
| | MNSTGFLKTCGAPPCNIYGGEGNPHNESDLFCPTDCFRKHPETT |
| | YSRCGAGPWLTPRCMVDYPYRLWHYPCTVDFRLFKVRMFVG |
| | GFEHRFTAACNWTRGERCDIEDRDRSEQHPLLHSTTELAILPCS |
| | FTPMPALSTGLIHLHQNIVDVQYLYGVGSGMVGWALKWEFVIL |
| | VFLLLADARVCVALWLMLMISQTEAALENLVTLNAVAAAGTH |
| | GIGWYLVAFCAAWYVRGKLVPLVTYSLTGLWSLALLVLLLPQ |
| | RAYAWSGEDSATLGAGVLVLFGFFTLSPWYKHWIGRLMWWN |
| | QYTICRCESALHVWVPPLLARGSRDGVILLTSLLYPSLIFDITKL |
| | LMAVLGPLYLIQATITTTPYFVRAHVLVRLCMLVRSVIGGKYFQ |
| | MIILSIGRWFNTYLYDHLAPMQHWAAAGLKDLAVATEPVIFSP |
| | MEIKVITWGADTAACGDILCGLPVSARLGREVLLGPADDYREM |
| | GWRLLAPITAYAQQTRGLLGTIVTSLTGRDKNVVTGEVQVLST |
| | ATQTFLGTTVGGVIWTVYHGAGSRTLAGAKHPALQMYTNVDQ |
| | DLVGWPAPPGAKSLEPCACGSSDLYLVTRDADVIPARRRGDST |
| | ASLLSPRPLACLKGSSGGPVMCPSGHVAGIFRAAVCTRGVAKS |
| | LQFIPVETLSTQARSPSFSDNSTPPAVPQSYQVGYLHAPTGSGKS |
| | TKVPAAYVAQGYNVLVLNPSVAATLGFGSFMSRAYGIDPNIRT |
| | GNRTVTTGAKLTYSTYGKFLADGGCSGGAYDVIICDECHAQDA |
| | TSILGIGTVLDQAETAGVRLTVLATATPPGSITVPHSNIEEVALG |
| | SEGEIPFYGKAIPIALLKGGRHLIFCHSKKKCDEIASKLRGMGLN |
| | AVAYYRGLDVSVIPTTGDVVVCATDALMTGFTGDFDSVIDCNV |
| | AVEQYVDFSLDPTFSIETRTAPQDAVSRSQRRGRTGRGRLGTYR |
| | YVASGERPSGMFDSVVLCECYDAGCSWYDLQPAETTVRLRAY |
| | LSTPGLPVCQDHLDFWESVFTGLTHIDAHFLSQTKQQGLNFSYL |
| | TAYQATVCARAQAPPPSWDEMWKCLVRLKPTLHGPTPLLYRL |
| | GPVQNETCLTHPITKYLMACMSADLEVTTSTWVLLGGVLAAL |
| | AAYCLSVGCVVIVGHIELEGKPALVPDKEVLYQQYDEMEECSQ |
| | AAPYIEQAQVIAHQFKEKILGLLQRATQQQAVIEPIVTTNWQKL |
| | EAFWHKHMWNFVSGIQYLAGLSTLPGNPAVASLMAFTASVTS |
| | PLTTNQTMFFNILGGWVATHLAGPQSSSAFVVSGLAGAAIGGIG |
| | LGRVLLDILAGYGAGVSGALVAFKIMGGECPTAEDMVNLLPAI |
| | LSPGALVVGVICAAILRRHVGPGEGAVQWMNRLIAFASRGNHV |
| | SPTHYVPESDAAARVTALLSSLTVTSLLRRLHQWINEDYPSPCS |
| | DDWLRTIWDWVCSVLADFKAWLSAKIMPALPGLPFISCQKGY |
| | KGVWRGDGVMSTRCPCGAAITGHVKNGSMRLAGPRTCANMW |
| | HGTFPINEYTTGPSTPCPSPNYTRALWRVAANSYVEVRRVGDF |
| | HYITGATEDELKCPCQVPAAEFFTEVDGVRLHRYAPPCKPLLRD |
| | DITFMVGLHSYTIGSQLPCEPEPDVSVLTSMLRDPSHITAETAAR |
| | RLARGSPPSEASSSASQLSAPSLKATCQTHRPHPDAELVDANLL |
| | WRQEMGSNITRVESETKVVVLDSFEPLRAETDDVEPSVAAEC |
| | FKKPPKYPPALPIWARPDYNPPLLDRWKAPDYVPPTVHGCALP |
| | PRGAPPVPPPRRKRTIQLDGSNVSAALAALAEKSFPSSKPQEENS |
| | SSSGVDTQSSTTSKVPPSPGGESDSESCSSMPPLEGEPGDPDLSC |
| | DSWSTVSDSEEQSVVCCSMSYSWTGALITPCSAEEEKLPISPLSN |
| | SLLRHHNLVYSTSSRSASQRQKKVTFDRLQVLDDHYKTALKEV |
| | KERASRVKARMLTIEEACALVPPHSARSKFGYSAKDVRSLSSR |
| | AINQIRSVWEDLLEDTTTPIPTTIMAKNEVFCVDPAKGGRKPAR |
| | LIVYPDLGVRVCEKRALYDVIQKLSIETMGPAYGFQYSPQQRVE |
| | RLLKMWTSKKTPLGFSYDTRCFDSTVTEQDIRVEEEIYQCCNLE |
| | PEARKVISSLTERLYCGGPMFNSKGAQCGYRRCRASGVLPTSFG |
| | NTITCYIKATAAAKAANLRNPDFLVCGDDLVVVAESDGVDEDR |
| | AALRAFTEAMTRYSAPPGDAPQATYDLELITSCSSNVSVARDD |
| | KGRRYYYLTRDATTPLARAAWETARHTPVNSWLGNIIMYAPTI |
| | WVRMVMMTHFFSILQSQEILDRPLDFEMYGATYSVTPLDLPAII |
| | ERLHGLSAFTLHSYSPVELNRVAGTLRKLGCPPLRAWRHRARA |
| | VRAKLIAQGGKAKICGLYLFNWAVRTKTNLTPLPAAGQLDLSS |
| | WFTVGVGGNDIYHSVSRARTRHLLLCLLLLTVGVGIFLLPAR |
| SEQ ID NO: 114 | ATGTTTGTGTTTCTGGTCTTACTGCCTCTGGTCTCCTCTCAGT |
| Omicron | GTGTTAACTTAATAACTAGGACTCAATCCTACACCAATTCCT |
| BA.2.75.2 spike | TCACACGAGGAGTGTATTATCCCGATAAAGTTTTCCGTTCAT |
| protein with 2P | CAGTTCTCCACTCTACCCAAGACCTGTTTCTGCCCTTCTTCTC |
| modification | AAACGTAACCTGGTTCCACGCTATCCATGTTTCCGGCACAAA |
| nucleic acid | CGGTACAAAACGGTTCGATAATCCCGTGTTGCCATTCAACGA |
| sequence | CGGTGTTTATTTTGCTTCCACCGAAAAGTCCAACATCATAAG |
| | GGGCTGGATTTTCGGAACCACTCTGGATTCTAAGACTCAGTC |
| | CCTGCTCATTGTGAATAACGCTACCAACGTCGTAATAAAAGT |
| | GTGCGAGTTCCAATTCTGTAATGATCCCTTTCTCGACGTCTA |
| | CTATCACGAAAATAATAAGTCTAGGATGGAATCCGAACTGA |
| | GAGTATACAGCTCCGCTAATAATTGTACGTTTGAATACGTCT |
| | CCCAACCGTTTCTCATGGATCTCGAAGGCAAACAAGGAAAC |
| | TTCAAGAATCTGCGGGAGTTCGTCTTCAAGAATATTGACGGG |
| | TATTTCAAAATTTATTCCAAACATACACCAGTTAACCTCGGA |
| | CGTGATCTTCCGCAAGGATTCTCCGCCCTGGAGCCGCTTGTC |
| | GACTTACCAATCGGGATAAATATAACCAGGTTCCAAACACT |
| | CTTAGCACTTCATCGTTCATATTTAACACCCGGCGACTCCTC |
| | ATCTAGTTGGACTGCTGGGGCCGCTGCATACTACGTCGGGTA |
| | TCTGCAACCACGGACATTTCTGCTCAAGTATAATGAGAATGG |
| | AACGATTACAGACGCAGTCGATTGTGCCTTGGATCCCCTCAG |
| | CGAAACAAAATGTACTCTCAAAAGCTTTACAGTAGAAAAGG |
| | GGATTTATCAAACCAGTAACTTCAGAGTCCAGCCAACAGAA |
| | TCCATAGTGCGCTTTCCTAATATTACCAATCTCTGTCCCTTCC |
| | ATGAAGTATTCAATGCTACTACCTTTGCTTCCGTCTACGCCT |
| | GGAATAGGAAAAGGATATCCAATTGCGTGGCCGATTATAGC |
| | GTTTTGTACAACTTTGCACCCTTCTTCGCTTTCAAGTGTTACG |
| | GCGTCAGTCCTACAAAACTGAATGACTTATGTTTCACCAATG |
| | TCTACGCCGATTCCTTCGTTATTCGAGGCAACGAAGTCAGTC |
| | AAATCGCACCTGGCCAAACAGGTAATATCGCAGATTACAAT |
| | TATAAATTACCAGACGACTTTACTGGGTGCGTGATTGCCTGG |
| | AATAGCAACAAACTCGATTCAAAAGTAAGTGGTAATTACAA |
| | CTATCTCTACAGATTGTTTCGGAAAAGTAAGCTCAAGCCTTT |
| | CGAACGTGACATAAGTACTGAGATCTACCAAGCGGGAAATA |
| | AACCTTGTAATGGAGTTGCAGGTTCCAACTGTTATTTCCCCC |
| | TGCAATCTTATGGCTTTCGTCCCACATACGGTGTAGGCCATC |
| | AGCCCTATCGCGTCGTAGTCCTCTCCTTTGAACTGCTCCACG |
| | CTCCTGCAACAGTGTGCGGTCCTAAGAAATCAACAAACCTG |
| | GTAAAGAATAAATGTGTCAATTTCAACTTCAATGGGCTGACT |
| | GGGACCGGAGTATTAACAGAAAGCAATAAGAAGTTTCTTCC |
| | GTTTCAGCAATTTGGAAGGGATATAGCCGACACCACGGACG |
| | CAGTAAGAGACCCACAAACACTTGAGATTTTGGATATCACC |
| | CCATGTTCATTCGGCGGTGTCTCTGTGATCACTCCCGGCACA |
| | AACACAAGCAACCAAGTTGCAGTACTGTACCAAGGCGTCAA |
| | TTGTACCGAAGTACCTGTCGCAATACACGCCGACCAACTCAC |
| | GCCTACTTGGCGGGTCTACTCCACTGGAAGCAACGTGTTCCA |
| | AACACGGGCCGGATGTTTGATCGGAGCCGAGTATGTGAATA |
| | ATTCCTATGAGTGCGACATCCCTATAGGAGCCGGCATTTGTG |
| | CGTCTTACCAAACACAAACCAAGAGCCATAGACGAGCAAGA |
| | AGTGTTGCTAGTCAGTCCATCATAGCCTACACAATGTCCCTT |
| | GGCGCAGAAAATAGTGTGGCCTATTCAAATAATTCCATCGCT |
| | ATCCCAACGAACTTTACCATAAGCGTCACTACTGAAATACTC |
| | CCTGTCTCCATGACCAAGACTAGCGTGGACTGTACCATGTAT |
| | ATATGTGGGGACAGCACAGAATGCTCCAATCTGTTGTTGCAA |
| | TATGGATCCTTTTGCACCCAGCTGAAGAGGGCCTTGACCGGC |
| | ATTGCGGTGGAACAAGATAAGAATACACAGGAAGTCTTTGC |
| | CCAAGTGAAACAAATATATAAAACCCCACCGATTAAGTACT |
| | TCGGCGGTTTCAACTTTAGCCAGATTCTCCCCGATCCCTCAA |
| | AACCTAGCAAGCGGTCATTTATTGAGGATCTGCTTTTCAATA |
| | AAGTGACCCTGGCCGATGCTGGCTTCATCAAACAATACGGC |
| | GACTGCCTGGGTGACATCGCCGCCAGGGATCTCATTTGCGCC |
| | CAGAAGTTTAACGGATTGACTGTACTGCCGCCCTTGCTTACC |
| | GACGAGATGATAGCTCAATACACCAGTGCGCTCCTTGCAGG |
| | GACCATCACGAGCGGATGGACATTTGGAGCTGGGGCCGCGC |
| | TTCAAATTCCTTTTGCAATGCAGATGGCTTACAGGTTCAACG |
| | GAATTGGGGTGACCCAAAACGTACTCTATGAGAATCAAAAG |
| | CTCATTGCTAACCAGTTCAATTCAGCCATTGGGAAAATTCAA |
| | GACAGCTTAAGTTCCACTGCAAGCGCGTTGGGGAAACTGCA |
| | AGATGTTGTGAACCACAATGCGCAGGCGCTCAACACTCTGG |
| | TGAAGCAGCTCAGCTCAAAGTTTGGCGCTATAAGTAGTGTTT |
| | TGAATGACATCCTGTCTCGGCTGGACCCACCCGAGGCCGAA |
| | GTGCAAATTGATCGTCTGATCACAGGAAGGCTTCAAAGCCT |
| | GCAGACGTATGTTACACAACAACTGATTCGGGCCGCAGAAA |
| | TAAGGGCATCTGCCAACTTGGCCGCTACCAAAATGTCTGAGT |
| | GTGTTTTGGGACAATCCAAACGGGTCGATTTCTGCGGTAAAG |
| | GATATCATCTCATGTCTTTCCCTCAGAGTGCCCCTCACGGCG |
| | TAGTCTTCTTACACGTAACCTACGTACCCGCACAAGAGAAGA |
| | ATTTCACTACCGCACCAGCAATCTGCCATGACGGGAAAGCC |
| | CACTTTCCGAGGGAAGGAGTATTCGTATCTAACGGCACGCA |
| | CTGGTTCGTGACCCAAAGAAATTTCTATGAGCCACAGATCAT |
| | AACTACAGACAATACTTTCGTTAGTGGTAACTGTGACGTTGT |
| | GATAGGAATAGTCAATAACACCGTGTACGATCCCCTTCAACC |
| | TGAGCTCGACTCCTTCAAGGAAGAGCTCGATAAGTATTTTAA |
| | GAATCACACTAGCCCGGATGTGGACTTGGGCGACATCAGCG |
| | GGATTAACGCAAGCGTAGTGAACATCCAGAAAGAGATTGAC |
| | AGGCTCAATGAAGTGGCCAAGAATCTCAACGAATCTCTGAT |
| | AAATCTCCAGGAACTGGGAAAGTATGAGCAATACATAAAGT |
| | GGCCATGGTATATCTGGCTGGGATTTATCGCAGGGCTGATAG |
| | CTATTGTTATGGTCACCATTATGTTGTGTTGTATGACGTCATG |
| | CTGCTCATGTCTGAAAGGTTGCTGTAGTTGCGGGAGTTGCTG |
| | CAAATTTGACGAAGACGATTCAGAGCCAGTCCTCAAGGGCG |
| | TTAAGCTTCACTACACTTAG |
| SEQ ID NO: 115 | MFVFLVLLPLVSSQCVNLITRTQSYTNSFTRGVYYPDKVFRSSV |
| Omicron | LHSTQDLFLPFFSNVTWFHAIHVSGTNGTKRFDNPVLPFNDGVY |
| BA.2.75.2 spike | FASTEKSNIIRGWIFGTTLDSKTQSLLIVNNATNVVIKVCEFQFC |
| protein with 2P | NDPFLDVYYHENNKSRMESELRVYSSANNCTFEYVSQPFLMDL |
| modification | EGKQGNFKNLREFVFKNIDGYFKIYSKHTPVNLGRDLPQGFSAL |
| amino acid | EPLVDLPIGINITRFQTLLALHRSYLTPGDSSSSWTAGAAAYYV |
| sequence | GYLQPRTFLLKYNENGTITDAVDCALDPLSETKCTLKSFTVEKG |
| | IYQTSNFRVQPTESIVRFPNITNLCPFHEVFNATTFASVYAWNRK |
| | RISNCVADYSVLYNFAPFFAFKCYGVSPTKLNDLCFTNVYADSF |
| | VIRGNEVSQIAPGQTGNIADYNYKLPDDFTGCVIAWNSNKLDS |
| | KVSGNYNYLYRLFRKSKLKPFERDISTEIYQAGNKPCNGVAGS |
| | NCYFPLQSYGFRPTYGVGHQPYRVVVLSFELLHAPATVCGPKK |
| | STNLVKNKCVNFNFNGLTGTGVLTESNKKFLPFQQFGRDIADT |
| | TDAVRDPQTLEILDITPCSFGGVSVITPGTNTSNQVAVLYQGVN |
| | CTEVPVAIHADQLTPTWRVYSTGSNVFQTRAGCLIGAEYVNNS |
| | YECDIPIGAGICASYQTQTKSHRRARSVASQSIIAYTMSLGAENS |
| | VAYSNNSIAIPTNFTISVTTEILPVSMTKTSVDCTMYICGDSTECS |
| | NLLLQYGSFCTQLKRALTGIAVEQDKNTQEVFAQVKQIYKTPPI |
| | KYFGGFNFSQILPDPSKPSKRSFIEDLLFNKVTLADAGFIKQYGD |
| | CLGDIAARDLICAQKFNGLTVLPPLLTDEMIAQYTSALLAGTITS |
| | GWTFGAGAALQIPFAMQMAYRFNGIGVTQNVLYENQKLIANQ |
| | FNSAIGKIQDSLSSTASALGKLQDVVNHNAQALNTLVKQLSSKF |
| | GAISSVLNDILSRLDPPEAEVQIDRLITGRLQSLQTYVTQQLIRA |
| | AEIRASANLAATKMSECVLGQSKRVDFCGKGYHLMSFPQSAPH |
| | GVVFLHVTYVPAQEKNFTTAPAICHDGKAHFPREGVFVSNGTH |
| | WFVTQRNFYEPQIITTDNTFVSGNCDVVIGIVNNTVYDPLQPEL |
| | DSFKEELDKYFKNHTSPDVDLGDISGINASVVNIQKEIDRLNEV |
| | AKNLNESLINLQELGKYEQYIKWPWYIWLGFIAGLIAIVMVTIM |
| | LCCMTSCCSCLKGCCSCGSCCKFDEDDSEPVLKGVKLHYT |
| SEQ ID NO: 116 | ATGTTTGTATTTCTCGTTCTGCTCCCTTTAGTGTCTAGCCAAT |
| Omicron-22E | GCGTAAACCTGATCACTCGCACCCAATCTTATACCAATTCTT |
| (BQ.1) spike | TTACTAGAGGAGTGTACTATCCCGATAAGGTTTTCCGCAGCT |
| protein with 2P | CCGTGCTCCATAGTACGCAGGATCTTTTCCTGCCATTCTTCA |
| modification | GTAACGTCACATGGTTTCATGCTATTTCAGGGACAAACGGGA |
| nucleic acid | CAAAGAGATTCGACAATCCCGTGCTGCCCTTCAATGACGGTG |
| sequence | TATATTTTGCCAGCACGGAGAAGAGTAACATAATAAGGGGA |
| | TGGATCTTTGGTACTACATTGGACTCAAAGACACAAAGCCTG |
| | TTGATTGTGAATAACGCCACCAACGTGGTGATCAAGGTCTGT |
| | GAATTTCAATTCTGTAATGACCCATTCCTCGACGTATATTAC |
| | CATAAGAACAACAAGAGTTGGATGGAGAGTGAATTTCGAGT |
| | CTATAGTAGTGCAAATAATTGCACTTTCGAATACGTTAGTCA |
| | ACCTTTCCTGATGGATCTGGAAGGGAAACAAGGCAACTTCA |
| | AGAATTTAAGAGAGTTCGTCTTCAAGAATATAGACGGATATT |
| | TCAAAATCTATTCCAAGCATACTCCAATCAATCTGGGCAGGG |
| | ATCTGCCCCAGGGCTTCAGCGCACTGGAGCCCCTCGTCGATT |
| | TGCCTATAGGCATCAATATAACAAGGTTTCAAACCCTTCTGG |
| | CCTTACATCGAAGCTATCTCACACCCGGCGACAGCAGTAGTG |
| | GATGGACAGCAGGAGCTGCTGCATATTACGTGGGATATCTTC |
| | AACCGAGAACCTTCTTATTAAAATACAATGAGAACGGGACT |
| | ATAACAGATGCAGTTGATTGTGCACTCGACCCCCTCAGCGAG |
| | ACAAAGTGTACACTGAAATCATTCACAGTCGAGAAAGGCAT |
| | CTACCAGACAAGCAATTTCCGAGTGCAGCCCACTGAATCTAT |
| | AGTGAGGTTCCCAAATATCACTAATTTGTGCCCATTTGACGA |
| | AGTATTCAATGCTACCACTTTTGCCTCTGTGTATGCATGGAA |
| | TCGCAAGAGAATTTCCAATTGCGTGGCTGATTACTCAGTGTT |
| | GTACAACTTTGCTCCATTCTTTGCTTTCAAGTGCTATGGAGTA |
| | TCCCCAACCAAATTAAACGATCTTTGTTTTACCAATGTCTAT |
| | GCTGATTCATTCGTAATTCGAGGGAATGAGGTTAGTCAAATT |
| | GCTCCAGGACAAACTGGCAACATTGCCGATTATAACTATAA |
| | ACTCCCAGACGATTTTACCGGGTGCGTGATCGCTTGGAACTC |
| | CAATAAGTTGGACAGTACTGTCGGCGGAAACTATAATTATA |
| | GATATCGCCTCTTCAGGAAAAGTAAGCTTAAACCCTTTGAAC |
| | GCGATATCTCCACCGAGATTTATCAAGCAGGTAATAAACCAT |
| | GCAACGGTGTAGCTGGAGTTAACTGTTATTTCCCTCTGCAGT |
| | CATACGGTTTTCGACCAACTTACGGGGTGGGCCATCAACCCT |
| | ACCGAGTTGTAGTGCTTAGCTTCGAACTGCTCCATGCTCCGG |
| | CCACCGTCTGTGGCCCCAAGAAATCTACTAATCTTGTAAAGA |
| | ATAAGTGTGTGAACTTCAATTTTAACGGTCTTACCGGCACAG |
| | GTGTGCTCACAGAGAGTAACAAGAAATTCCTTCCATTTCAAC |
| | AATTCGGGAGAGACATTGCTGATACCACCGATGCAGTGCGC |
| | GACCCCCAAACGTTAGAGATACTTGATATAACCCCATGTTCA |
| | TTCGGTGGAGTATCAGTGATCACTCCTGGTACTAATACTTCT |
| | AACCAAGTTGCTGTCTTATACCAGGGCGTAAATTGTACAGAA |
| | GTGCCGGTCGCGATCCATGCGGACCAACTCACCCCTACATGG |
| | CGCGTGTACTCCACGGGGAGTAACGTGTTTCAAACCAGGGC |
| | AGGCTGCCTCATTGGTGCTGAGTATGTGAACAACTCCTACGA |
| | GTGCGATATCCCTATAGGTGCCGGGATTTGCGCCTCATATCA |
| | GACCCAAACTAAAAGTCATCGCAGGGCCAGGTCTGTAGCAT |
| | CTCAAAGTATAATTGCATATACAATGTCACTTGGCGCTGAGA |
| | ATAGCGTTGCGTATTCTAATAATAGCATTGCCATACCTACCA |
| | ATTTCACTATCTCCGTGACAACAGAGATTCTGCCAGTCTCTA |
| | TGACCAAAACATCAGTCGACTGCACGATGTATATCTGCGGG |
| | GATAGTACCGAGTGCTCTAATCTGCTGCTCCAATATGGAAGT |
| | TTCTGTACTCAGCTTAAAAGGGCTCTTACTGGTATCGCCGTA |
| | GAGCAAGATAAGAATACGCAGGAAGTCTTTGCGCAAGTTAA |
| | ACAAATTTACAAGACACCGCCGATAAAATATTTTGGCGGTTT |
| | CAACTTTTCCCAGATCCTGCCCGATCCCTCAAAACCAAGCAA |
| | ACGCTCTTTCATCGAAGACCTGTTGTTTAATAAAGTGACCTT |
| | GGCCGATGCTGGGTTTATCAAACAATACGGTGACTGCCTTGG |
| | GGACATAGCAGCTAGGGACTTAATCTGCGCCCAGAAGTTCA |
| | ATGGACTGACTGTTCTCCCACCGTTGTTGACAGACGAAATGA |
| | TAGCCCAATACACGTCCGCTCTCCTGGCTGGAACAATAACCT |
| | CAGGGTGGACCTTCGGGGCTGGCGCTGCTCTGCAAATTCCTT |
| | TCGCAATGCAAATGGCTTACCGTTTTAATGGCATCGGCGTTA |
| | CCCAGAACGTACTGTACGAGAATCAAAAGCTGATTGCAAAT |
| | CAATTCAATAGTGCCATCGGCAAGATTCAAGATAGCCTGTCC |
| | TCTACGGCAAGCGCTTTGGGTAAACTTCAAGACGTGGTGAAT |
| | CATAATGCTCAAGCCCTTAACACACTGGTGAAGCAACTCTCC |
| | AGCAAGTTTGGCGCAATTAGCTCTGTACTTAATGATATTCTG |
| | AGCCGGCTGGACCCACCAGAGGCCGAAGTCCAAATAGACCG |
| | TCTGATTACGGGACGGCTTCAATCCTTGCAGACCTACGTTAC |
| | CCAACAACTTATCAGGGCTGCCGAGATAAGAGCAAGTGCCA |
| | ACCTTGCTGCCACCAAGATGTCCGAGTGTGTACTCGGCCAAT |
| | CCAAGCGCGTCGACTTTTGTGGGAAGGGATATCACTTGATGT |
| | CTTTCCCTCAATCTGCCCCTCACGGCGTGGTGTTCCTGCATGT |
| | GACCTACGTGCCAGCGCAAGAAAAGAACTTTACCACTGCTC |
| | CAGCAATTTGTCACGACGGCAAAGCACATTTTCCCAGGGAA |
| | GGAGTGTTTGTTTCTAATGGAACGCACTGGTTCGTAACACAA |
| | AGAAATTTCTATGAACCCCAAATCATTACTACTGATAACACC |
| | TTCGTTTCTGGTAACTGTGACGTAGTTATCGGAATCGTAAAC |
| | AACACTGTATATGACCCTCTGCAGCCAGAGCTCGACAGTTTT |
| | AAGGAAGAGTTGGATAAGTATTTTAAGAATCATACCTCTCCT |
| | GACGTCGACCTCGGCGACATTTCCGGCATAAATGCCTCAGTC |
| | GTGAATATCCAGAAAGAAATAGACCGCTTGAACGAGGTTGC |
| | GAAGAATTTGAACGAGTCCTTGATAGACCTGCAAGAGCTGG |
| | GAAAGTATGAGCAATACATCAAGTGGCCGTGGTATATATGG |
| | CTCGGGTTTATTGCTGGTTTAATCGCTATCGTGATGGTGACA |
| | ATAATGCTTTGTTGTATGACATCATGTTGCAGTIGTCTCAAA |
| | GGGTGTTGCAGTTGCGGCTCCTGCTGCAAGTTCGATGAGGAC |
| | GATTCTGAACCAGTGCTCAAAGGGGTTAAGCTTCATTATACG |
| | TAG |
| SEQ ID NO: 117 | MFVFLVLLPLVSSQCVNLITRTQSYTNSFTRGVYYPDKVFRSSV |
| Omicron-22E | LHSTQDLFLPFFSNVTWFHAISGTNGTKRFDNPVLPFNDGVYFA |
| (BQ.1) spike | STEKSNIIRGWIFGTTLDSKTQSLLIVNNATNVVIKVCEFQFCND |
| protein with 2P | PFLDVYYHKNNKSWMESEFRVYSSANNCTFEYVSQPFLMDLE |
| modification | GKQGNFKNLREFVFKNIDGYFKIYSKHTPINLGRDLPQGFSALE |
| amino acid | PLVDLPIGINITRFQTLLALHRSYLTPGDSSSGWTAGAAAYYVG |
| sequence | YLQPRTFLLKYNENGTITDAVDCALDPLSETKCTLKSFTVEKGI |
| | YQTSNFRVQPTESIVRFPNITNLCPFDEVFNATTFASVYAWNRK |
| | RISNCVADYSVLYNFAPFFAFKCYGVSPTKLNDLCFTNVYADSF |
| | VIRGNEVSQIAPGQTGNIADYNYKLPDDFTGCVIAWNSNKLDST |
| | VGGNYNYRYRLFRKSKLKPFERDISTEIYQAGNKPCNGVAGVN |
| | CYFPLQSYGFRPTYGVGHQPYRVVVLSFELLHAPATVCGPKKS |
| | TNLVKNKCVNFNFNGLTGTGVLTESNKKFLPFQQFGRDIADTT |
| | DAVRDPQTLEILDITPCSFGGVSVITPGTNTSNQVAVLYQGVNC |
| | TEVPVAIHADQLTPTWRVYSTGSNVFQTRAGCLIGAEYVNNSY |
| | ECDIPIGAGICASYQTQTKSHRRARSVASQSIIAYTMSLGAENSV |
| | AYSNNSIAIPTNFTISVTTEILPVSMTKTSVDCTMYICGDSTECSN |
| | LLLQYGSFCTQLKRALTGIAVEQDKNTQEVFAQVKQIYKTPPIK |
| | YFGGFNFSQILPDPSKPSKRSFIEDLLFNKVTLADAGFIKQYGDC |
| | LGDIAARDLICAQKFNGLTVLPPLLTDEMIAQYTSALLAGTITSG |
| | WTFGAGAALQIPFAMQMAYRENGIGVTQNVLYENQKLIANQF |
| | NSAIGKIQDSLSSTASALGKLQDVVNHNAQALNTLVKQLSSKF |
| | GAISSVLNDILSRLDPPEAEVQIDRLITGRLQSLQTYVTQQLIRA |
| | AEIRASANLAATKMSECVLGQSKRVDFCGKGYHLMSFPQSAPH |
| | GVVFLHVTYVPAQEKNFTTAPAICHDGKAHFPREGVFVSNGTH |
| | WFVTQRNFYEPQIITTDNTFVSGNCDVVIGIVNNTVYDPLQPEL |
| | DSFKEELDKYFKNHTSPDVDLGDISGINASVVNIQKEIDRLNEV |
| | AKNLNESLIDLQELGKYEQYIKWPWYIWLGFIAGLIAIVMVTIM |
| | LCCMTSCCSCLKGCCSCGSCCKFDEDDSEPVLKGVKLHYT |
| SEQ ID NO: 118 | CGTATAATGAAGCTTAACCCCTTTGAGCACTGGTTCAGAATC |
| Omicron-22E | GTCCTCATCGAACTTGCAGCAGGAGCCGCAACTGCAACACC |
| (BQ.1) spike | CTTTGAGACAACTGCAACATGATGTCATACAACAAAGCATT |
| protein with 2P | ATTGTCACCATCACGATAGCGATTAAACCAGCAATAAACCC |
| modification and | GAGCCATATATACCACGGCCACTTGATGTATTGCTCATACTT |
| furin cleavage | TCCCAGCTCTTGCAGGTCTATCAAGGACTCGTTCAAATTCTT |
| mutation nucleic | CGCAACCTCGTTCAAGCGGTCTATTTCTTTCTGGATATTCAC |
| acid sequence | GACTGAGGCATTTATGCCGGAAATGTCGCCGAGGTCGACGT |
| | CAGGAGAGGTATGATTCTTAAAATACTTATCCAACTCTTCCT |
| | TAAAACTGTCGAGCTCTGGCTGCAGAGGGTCATATACAGTGT |
| | TGTTTACGATTCCGATAACTACGTCACAGTTACCAGAAACGA |
| | AGGTGTTATCAGTAGTAATGATTTGGGGTTCATAGAAATTTC |
| | TTTGTGTTACGAACCAGTGCGTTCCATTAGAAACAAACACTC |
| | CTTCCCTGGGAAAATGTGCTTTGCCGTCGTGACAAATTGCTG |
| | GAGCAGTGGTAAAGTTCTTTTCTTGCGCTGGCACGTAGGTCA |
| | CATGCAGGAACACCACGCCGTGAGGGGCAGATTGAGGGAAA |
| | GACATCAAGTGATATCCCTTCCCACAAAAGTCGACGCGCTTG |
| | GATTGGCCGAGTACACACTCGGACATCTTGGTGGCAGCAAG |
| | GTTGGCACTTGCTCTTATCTCGGCAGCCCTGATAAGTTGTTG |
| | GGTAACGTAGGTCTGCAAGGATTGAAGCCGTCCCGTAATCA |
| | GACGGTCTATTTGGACTTCGGCCTCTGGTGGGTCCAGCCGGC |
| | TCAGAATATCATTAAGTACAGAGCTAATTGCGCCAAACTTGC |
| | TGGAGAGTTGCTTCACCAGTGTGTTAAGGGCTTGAGCATTAT |
| | GATTCACCACGTCTTGAAGTTTACCCAAAGCGCTTGCCGTAG |
| | AGGACAGGCTATCTTGAATCTTGCCGATGGCACTATTGAATT |
| | GATTTGCAATCAGCTTTTGATTCTCGTACAGTACGTTCTGGG |
| | TAACGCCGATGCCATTAAAACGGTAAGCCATTTGCATTGCGA |
| | AAGGAATTTGCAGAGCAGCGCCAGCCCCGAAGGTCCACCCT |
| | GAGGTTATTGTTCCAGCCAGGAGAGCGGACGTGTATTGGGC |
| | TATCATTTCGTCTGTCAACAACGGTGGGAGAACAGTCAGTCC |
| | ATTGAACTTCTGGGCGCAGATTAAGTCCCTAGCTGCTATGTC |
| | CCCAAGGCAGTCACCGTATTGTTTGATAAACCCAGCATCGGC |
| | CAAGGTCACTTTATTAAACAACAGGTCTTCGATGAAAGAGC |
| | GTTTGCTTGGTTTTGAGGGATCGGGCAGGATCTGGGAAAAGT |
| | TGAAACCGCCAAAATATTTTATCGGCGGTGTCTTGTAAATTT |
| | GTTTAACTTGCGCAAAGACTTCCTGCGTATTCTTATCTTGCTC |
| | TACGGCGATACCAGTAAGAGCCCTTTTAAGCTGAGTACAGA |
| | AACTTCCATATTGGAGCAGCAGATTAGAGCACTCGGTACTAT |
| | CCCCGCAGATATACATCGTGCAGTCGACTGATGTTTTGGTCA |
| | TAGAGACTGGCAGAATCTCTGTTGTCACGGAGATAGTGAAA |
| | TTGGTAGGTATGGCAATGCTATTATTAGAATACGCAACGCTA |
| | TTCTCAGCGCCAAGTGACATTGTATATGCAATTATACTTTGA |
| | GATGCTACAGAGCTGGCGCTCCCATGACTTTTAGTTTGGGTC |
| | TGATATGAGGCGCAAATCCCGGCACCTATAGGGATATCGCA |
| | CTCGTAGGAGTTGTTCACATACTCAGCACCAATGAGGCAGCC |
| | TGCCCTGGTTTGAAACACGTTACTCCCCGTGGAGTACACGCG |
| | CCATGTAGGGGTGAGTTGGTCCGCATGGATCGCGACCGGCA |
| | CTTCTGTACAATTTACGCCCTGGTATAAGACAGCAACTTGGT |
| | TAGAAGTATTAGTACCAGGAGTGATCACTGATACTCCACCG |
| | AATGAACATGGGGTTATATCAAGTATCTCTAACGTTTGGGGG |
| | TCGCGCACTGCATCGGTGGTATCAGCAATGTCTCTCCCGAAT |
| | TGTTGAAATGGAAGGAATTTCTTGTTACTCTCTGTGAGCACA |
| | CCTGTGCCGGTAAGACCGTTAAAATTGAAGTTCACACACTTA |
| | TTCTTTACAAGATTAGTAGATTTCTTGGGGCCACAGACGGTG |
| | GCCGGAGCATGGAGCAGTTCGAAGCTAAGCACTACAACTCG |
| | GTAGGGTTGATGGCCCACCCCGTAAGTTGGTCGAAAACCGT |
| | ATGACTGCAGAGGGAAATAACAGTTAACTCCAGCTACACCG |
| | TTGCATGGTTTATTACCTGCTTGATAAATCTCGGTGGAGATA |
| | TCGCGTTCAAAGGGTTTAAGCTTACTTTTCCTGAAGAGGCGA |
| | TATCTATAATTATAGTTTCCGCCGACAGTACTGTCCAACTTA |
| | TTGGAGTTCCAAGCGATCACGCACCCGGTAAAATCGTCTGG |
| | GAGTTTATAGTTATAATCGGCAATGTTGCCAGTTTGTCCTGG |
| | AGCAATTTGACTAACCTCATTCCCTCGAATTACGAATGAATC |
| | AGCATAGACATTGGTAAAACAAAGATCGTTTAATTTGGTTGG |
| | GGATACTCCATAGCACTTGAAAGCAAAGAATGGAGCAAAGT |
| | TGTACAACACTGAGTAATCAGCCACGCAATTGGAAATTCTCT |
| | TGCGATTCCATGCATACACAGAGGCAAAAGTGGTAGCATTG |
| | AATACTTCGTCAAATGGGCACAAATTAGTGATATTTGGGAAC |
| | CTCACTATAGATTCAGTGGGCTGCACTCGGAAATTGCTTGTC |
| | TGGTAGATGCCTTTCTCGACTGTGAATGATTTCAGTGTACAC |
| | TTTGTCTCGCTGAGGGGGTCGAGTGCACAATCAACTGCATCT |
| | GTTATAGTCCCGTTCTCATTGTATTTTAATAAGAAGGTTCTCG |
| | GTTGAAGATATCCCACGTAATATGCAGCAGCTCCTGCTGTCC |
| | ATCCACTACTGCTGTCGCCGGGTGTGAGATAGCTTCGATGTA |
| | AGGCCAGAAGGGTTTGAAACCTTGTTATATTGATGCCTATAG |
| | GCAAATCGACGAGGGGCTCCAGTGCGCTGAAGCCCTGGGGC |
| | AGATCCCTGCCCAGATTGATTGGAGTATGCTTGGAATAGATT |
| | TTGAAATATCCGTCTATATTCTTGAAGACGAACTCTCTTAAA |
| | TTCTTGAAGTTGCCTTGTTTCCCTTCCAGATCCATCAGGAAA |
| | GGTTGACTAACGTATTCGAAAGTGCAATTATTTGCACTACTA |
| | TAGACTCGAAATTCACTCTCCATCCAACTCTTGTTGTTCTTAT |
| | GGTAATATACGTCGAGGAATGGGTCATTACAGAATTGAAAT |
| | TCACAGACCTTGATCACCACGTTGGTGGCGTTATTCACAATC |
| | AACAGGCTTTGTGTCTTTGAGTCCAATGTAGTACCAAAGATC |
| | CATCCCCTTATTATGTTACTCTTCTCCGTGCTGGCAAAATATA |
| | CACCGTCATTGAAGGGCAGCACGGGATTGTCGAATCTCTTTG |
| | TCCCGTTTGTCCCTGAAATAGCATGAAACCATGTGACGTTAC |
| | TGAAGAATGGCAGGAAAAGATCCTGCGTACTATGGAGCACG |
| | GAGCTGCGGAAAACCTTATCGGGATAGTACACTCCTCTAGTA |
| | AAAGAATTGGTATAAGATTGGGTGCGAGTGATCAGGTTTAC |
| | GCATTGGCTAGACACTAAAGGGAGCAGAACGAGAAATACAA |
| | ACAT |
| SEQ ID NO: 119 | MFVFLVLLPLVSSQCVNLITRTQSYTNSFTRGVYYPDKVFRSSV |
| Omicron-22E | LHSTQDLFLPFFSNVTWFHAISGTNGTKRFDNPVLPFNDGVYFA |
| (BQ.1) spike | STEKSNIIRGWIFGTTLDSKTQSLLIVNNATNVVIKVCEFQFCND |
| protein with 2P | PFLDVYYHKNNKSWMESEFRVYSSANNCTFEYVSQPFLMDLE |
| modification and | GKQGNFKNLREFVFKNIDGYFKIYSKHTPINLGRDLPQGFSALE |
| furin cleavage | PLVDLPIGINITRFQTLLALHRSYLTPGDSSSGWTAGAAAYYVG |
| mutation amino | YLQPRTFLLKYNENGTITDAVDCALDPLSETKCTLKSFTVEKGI |
| acid sequence | YQTSNFRVQPTESIVRFPNITNLCPFDEVFNATTFASVYAWNRK |
| | RISNCVADYSVLYNFAPFFAFKCYGVSPTKLNDLCFTNVYADSF |
| | VIRGNEVSQIAPGQTGNIADYNYKLPDDFTGCVIAWNSNKLDST |
| | VGGNYNYRYRLFRKSKLKPFERDISTEIYQAGNKPCNGVAGVN |
| | CYFPLQSYGFRPTYGVGHQPYRVVVLSFELLHAPATVCGPKKS |
| | TNLVKNKCVNFNFNGLTGTGVLTESNKKFLPFQQFGRDIADTT |
| | DAVRDPQTLEILDITPCSFGGVSVITPGTNTSNQVAVLYQGVNC |
| | TEVPVAIHADQLTPTWRVYSTGSNVFQTRAGCLIGAEYVNNSY |
| | ECDIPIGAGICASYQTQTKSHGSASSVASQSIIAYTMSLGAENSV |
| | AYSNNSIAIPTNFTISVTTEILPVSMTKTSVDCTMYICGDSTECSN |
| | LLLQYGSFCTQLKRALTGIAVEQDKNTQEVFAQVKQIYKTPPIK |
| | YFGGFNFSQILPDPSKPSKRSFIEDLLFNKVTLADAGFIKQYGDC |
| | LGDIAARDLICAQKFNGLTVLPPLLTDEMIAQYTSALLAGTITSG |
| | WTFGAGAALQIPFAMQMAYRFNGIGVTQNVLYENQKLIANQF |
| | NSAIGKIQDSLSSTASALGKLQDVVNHNAQALNTLVKQLSSKF |
| | GAISSVLNDILSRLDPPEAEVQIDRLITGRLQSLQTYVTQQLIRA |
| | AEIRASANLAATKMSECVLGQSKRVDFCGKGYHLMSFPQSAPH |
| | GVVFLHVTYVPAQEKNFTTAPAICHDGKAHFPREGVFVSNGTH |
| | WFVTQRNFYEPQIITTDNTFVSGNCDVVIGIVNNTVYDPLQPEL |
| | DSFKEELDKYFKNHTSPDVDLGDISGINASVVNIQKEIDRLNEV |
| | AKNLNESLIDLQELGKYEQYIKWPWYIWLGFIAGLIAIVMVTIM |
| | LCCMTSCCSCLKGCCSCGSCCKFDEDDSEPVLKGVKLHYT |
| SEQ ID NO: 120 | ATGTTCGGTTTTCTGGTCCTCTTACCCCTTGTGTCCTCACAAT |
| Codon optimized | GCGTCAACCTGATAACCAGGACACAAAGTTATACCAACTCC |
| Omicron BA.5 | TTTACTCGTGGTGTCTACTACCCTGATAAAGTATTTAGAAGC |
| spike protein | AGCGTGCTGCATAGCACACAGGATCTGTTCCTGCCCTTCTTC |
| with 2P | TCCAACGTCACATGGTTCCACGCAATCAGTGGCACGAACGG |
| modification | AACCAAGAGATTTGATAATCCGGTTCTCCCCTTCAACGACGG |
| nucleic acid | CGTCTATTTTGCTTCTACAGAGAAAAGCAACATTATTCGAGG |
| sequence | TTGGATTTTCGGGACCACTCTTGATTCCAAAACACAAAGTCT |
| | CCTCATCGTAAATAACGCCACGAATGTGGTCATAAAAGTTTG |
| | CGAATTTCAGTTTTGCAACGATCCTTTCCTGGATGTCTACTAT |
| | CACAAGAACAACAAAAGTTGGATGGAATCCGAATTTCGAGT |
| | ATATTCCTCAGCAAACAACTGCACTTTTGAGTATGTGTCTCA |
| | ACCATTCCTGATGGATCTCGAAGGCAAACAAGGAAACTTTA |
| | AGAACCTGCGCGAGTTTGTCTTTAAGAACATAGACGGGTATT |
| | TCAAAATTTATTCTAAACATACGCCGATTAACTTGGGGCGCG |
| | ATCTCCCGCAAGGGTTTTCCGCCTTAGAGCCCCTCGTCGATC |
| | TGCCTATTGGAATCAACATCACACGGTTTCAGACTTTACTTG |
| | CACTGCACAGAAGTTATCTCACACCCGGAGACTCCTCATCAG |
| | GATGGACTGCGGGTGCTGCTGCTTATTACGTCGGGTATCTGC |
| | AACCAAGAACATTTCTTCTGAAATATAATGAGAATGGCACT |
| | ATTACGGATGCCGTGGATTGCGCACTTGACCCCCTCTCCGAA |
| | ACAAAATGTACTCTGAAGAGTTTTACAGTGGAAAAGGGAAT |
| | TTATCAAACAAGCAATTTCCGGGTGCAACCGACAGAAAGTA |
| | TCGTGAGGTTTCCAAACATTACAAATCTTTGCCCATTCGACG |
| | AGGTATTCAACGCTACTCGGTTTGCTTCTGTTTATGCATGGA |
| | ACCGGAAGAGGATCTCCAATTGTGTAGCAGATTACAGCGTG |
| | CTTTACAACTTCGCTCCATTCTTCGCATTTAAATGTTATGGCG |
| | TTTCTCCTACTAAACTTAATGATCTGTGCTTCACAAATGTGTA |
| | TGCCGACAGCTTCGTTATTAGGGGTAATGAGGTATCCCAGAT |
| | TGCTCCCGGACAAACAGGGAACATCGCGGACTATAACTACA |
| | AGTTGCCTGATGATTTTACTGGCTGCGTAATTGCCTGGAACT |
| | CTAATAAGCTTGACTCCAAAGTAGGCGGGAATTATAACTATC |
| | GATATAGACTGTTTCGCAAGTCTAACCTTAAACCCTTTGAGC |
| | GGGATATCTCAACAGAAATTTATCAAGCCGGAAATAAGCCC |
| | TGCAATGGGGTTGCAGGAGTTAATTGTTATTTCCCCCTGCAA |
| | AGTTACGGGTTCCGCCCCACATATGGGGTCGGTCACCAACCA |
| | TACCGGGTCGTCGTTCTGTCATTCGAATTGTTACATGCACCC |
| | GCTACCGTATGCGGCCCTAAGAAGTCAACTAATCTTGTCAAG |
| | AATAAATGCGTAAATTTTAACTTTAATGGATTGACTGGGACA |
| | GGTGTGTTGACCGAGTCAAACAAGAAGTTTCTGCCCTTTCAA |
| | CAATTTGGACGGGACATAGCCGATACGACAGATGCGGTTCG |
| | GGACCCGCAAACACTGGAAATACTGGATATAACTCCGTGTA |
| | GTTTTGGCGGTGTTAGCGTAATTACACCCGGTACAAATACAA |
| | GCAACCAAGTAGCAGTCCTGTATCAAGGCGTTAACTGTACTG |
| | AGGTACCAGTGGCAATTCACGCAGATCAGCTCACACCCACC |
| | TGGCGAGTGTACTCTACCGGATCTAATGTGTTCCAAACTCGT |
| | GCCGGATGCCTGATAGGAGCTGAGTACGTGAATAATAGTTA |
| | TGAGTGCGATATACCCATTGGGGCCGGCATATGCGCATCCTA |
| | CCAAACCCAAACCAAATCTCATAGGCGTGCTAGAAGTGTGG |
| | CCAGCCAATCTATCATCGCTTATACAATGTCCCTTGGCGCAG |
| | AAAATTCCGTGGCTTACTCAAACAACTCCATAGCCATTCCCA |
| | CTAATTTTACGATTAGTGTTACTACTGAGATCCTGCCGGTGT |
| | CTATGACTAAAACCTCTGTAGATTGTACGATGTACATCTGTG |
| | GGGACTCCACTGAATGCTCCAATCTGCTCCTGCAATACGGGA |
| | GCTTCTGTACCCAACTGAAACGAGCACTGACTGGCATTGCCG |
| | TGGAACAAGACAAGAACACACAAGAAGTATTCGCACAAGTA |
| | AAACAAATTTATAAGACACCCCCAATCAAATATTTCGGTGGT |
| | TTCAACTTTAGCCAGATCTTACCCGATCCAAGCAAACCCTCA |
| | AAACGGAGCTTTATTGAGGACTTACTCTTTAATAAAGTTACC |
| | TTGGCGGACGCTGGATTTATTAAACAGTACGGGGATTGTCTG |
| | GGTGATATTGCAGCACGCGATTTAATTTGTGCCCAAAAGTTC |
| | AATGGTCTTACCGTTCTGCCGCCTCTGTTAACAGACGAGATG |
| | ATCGCCCAATATACCTCTGCCCTGCTGGCAGGCACTATAACT |
| | TCCGGTTGGACGTTTGGCGCTGGGGCCGCTCTCCAGATCCCA |
| | TTTGCTATGCAAATGGCTTATCGGTTCAACGGCATTGGGGTT |
| | ACACAGAACGTGTTGTATGAGAACCAGAAATTAATTGCCAA |
| | CCAGTTTAATTCTGCCATCGGTAAGATACAAGATTCATTGTC |
| | TTCTACTGCTAGTGCACTCGGAAAGTTGCAAGACGTGGTCAA |
| | TCACAATGCACAGGCCTTAAATACTCTGGTTAAACAACTCAG |
| | CTCTAAATTCGGTGCCATTTCATCTGTCCTGAATGATATTTTG |
| | TCAAGACTCGACAAGGTAGAGGCGGAAGTACAAATTGATAG |
| | GCTCATCACAGGGCGACTCCAGTCATTGCAGACATACGTGA |
| | CACAACAGCTGATTCGCGCAGCGGAAATAAGGGCCTCAGCA |
| | AATCTGGCTGCAACCAAGATGTCAGAATGCGTTTTAGGTCAA |
| | AGTAAACGGGTTGATTTCTGTGGTAAGGGATACCATCTCATG |
| | TCATTCCCACAAAGCGCCCCTCACGGCGTTGTTTTCTTGCAT |
| | GTCACATATGTGCCAGCCCAAGAAAAGAATTTTACAACTGC |
| | CCCCGCCATTTGTCACGATGGTAAAGCTCATTTTCCACGCGA |
| | GGGTGTTTTCGTGTCAAATGGAACCCATTGGTTCGTCACTCA |
| | ACGCAATTTCTATGAACCCCAAATCATAACCACTGACAATAC |
| | GTTTGTTTCAGGCAATTGCGACGTGGTCATAGGAATTGTTAA |
| | CAATACTGTTTATGATCCGCTGCAACCCGAGTTGGATTCCTT |
| | TAAGGAAGAGTTGGACAAGTATTTTAAGAACCACACCAGCC |
| | CGGATGTTGATCTGGGAGATATTTCAGGAATCAATGCAAGC |
| | GTGGTTAATATCCAAAAGGAGATCGATCGTCTCAATGAAGT |
| | GGCCAAGAATTTGAATGAAAGTTTAATTGACCTTCAAGAACT |
| | CGGGAAGTACGAACAATATATTAAATGGCCCTGGTACATAT |
| | GGCTTGGCTTCATCGCCGGCCTCATTGCCATCGTGATGGTTA |
| | CAATAATGTTATGTTGCATGACATCTTGCTGCTCCTGTCTCA |
| | AGGGATGTTGCTCTTGTGGTAGCTGTTGTAAATTCGATGAGG |
| | ATGATTCTGAACCAGTGCTTAAGGGAGTCAAACTTCACTATA |
| | CATGA |
| SEQ ID NO: 121 | MFGFLVLLPLVSSQCVNLITRTQSYTNSFTRGVYYPDKVFRSSV |
| Omicron BA.5 | LHSTQDLFLPFFSNVTWFHAISGTNGTKRFDNPVLPFNDGVYFA |
| spike protein | STEKSNIIRGWIFGTTLDSKTQSLLIVNNATNVVIKVCEFQFCND |
| with 2P | PFLDVYYHKNNKSWMESEFRVYSSANNCTFEYVSQPFLMDLE |
| modification | GKQGNFKNLREFVFKNIDGYFKIYSKHTPINLGRDLPQGFSALE |
| amino acid | PLVDLPIGINITRFQTLLALHRSYLTPGDSSSGWTAGAAAYYVG |
| sequence | YLQPRTFLLKYNENGTITDAVDCALDPLSETKCTLKSFTVEKGI |
| | YQTSNFR VQPTESIVRFPNITNLCPFDEVFNATRFASVYAWNRK |
| | RISNCVADYSVLYNFAPFFAFKCYGVSPTKLNDLCFTNVYADSF |
| | VIRGNEVSQIAPGQTGNIADYNYKLPDDFTGCVIAWNSNKLDS |
| | KVGGNYNYRYRLFRKSNLKPFERDISTEIYQAGNKPCNGVAGV |
| | NCYFPLQSYGFRPTYGVGHQPYRVVVLSFELLHAPATVCGPKK |
| | STNLVKNKCVNFNFNGLTGTGVLTESNKKFLPFQQFGRDIADT |
| | TDAVRDPQTLEILDITPCSFGGVSVITPGTNTSNQVAVLYQGVN |
| | CTEVPVAIHADQLTPTWRVYSTGSNVFQTRAGCLIGAEYVNNS |
| | YECDIPIGAGICASYQTQTKSHRRARSVASQSIIAYTMSLGAENS |
| | VAYSNNSIAIPTNFTISVTTEILPVSMTKTSVDCTMYICGDSTECS |
| | NLLLQYGSFCTQLKRALTGIAVEQDKNTQEVFAQVKQIYKTPPI |
| | KYFGGFNFSQILPDPSKPSKRSFIEDLLFNKVTLADAGFIKQYGD |
| | CLGDIAARDLICAQKFNGLTVLPPLLTDEMIAQYTSALLAGTITS |
| | GWTFGAGAALQIPFAMQMAYRENGIGVTQNVLYENQKLIANQ |
| | FNSAIGKIQDSLSSTASALGKLQDVVNHNAQALNTLVKQLSSKF |
| | GAISSVLNDILSRLDKVEAEVQIDRLITGRLQSLQTYVTQQLIRA |
| | AEIRASANLAATKMSECVLGQSKRVDFCGKGYHLMSFPQSAPH |
| | GVVFLHVTYVPAQEKNFTTAPAICHDGKAHFPREGVFVSNGTH |
| | WFVTQRNFYEPQIITTDNTFVSGNCDVVIGIVNNTVYDPLQPEL |
| | DSFKEELDKYFKNHTSPDVDLGDISGINASVVNIQKEIDRLNEV |
| | AKNLNESLIDLQELGKYEQYIKWPWYIWLGFIAGLIAIVMVTIM |
| | LCCMTSCCSCLKGCCSCGSCCKFDEDDSEPVLKGVKLHYT |
| SEQ ID NO: 122 | ATGAGCGACAACGGCCCCCAAAACCAGAGGAACGCACTGCG |
| Omicron BQ.1 | GATAACATTCGGTGGTCCCTCAGATTCTACAGGAAGTAACCA |
| Nucleocapsid | AAACGGCGGAGCTCGGAGTAAGCAAAGAAGGCCGCAAGGC |
| nucleic acid | CTGCCTAACAACACTGCGTCTTGGTTCACTGCCCTGACTCAA |
| sequence | CACGGCAAAGAGGGACTGAAATTCCCCAGAGGTCAAGGGGT |
| | GCCAATCAACACTAACAGCTCCCCTGATGACCAAATTGGCTA |
| | TTACCGGAGAGCTACGCGACGGATCAGGGGCGGCGACGGCA |
| | AAATGAAGGACCTTAGCCCCCGCTGGTACTTCTACTATTTAG |
| | GTACCGGACCCGAAGCCGGCCTTCCCTACGGCGCTAATAAA |
| | GACGGCATTATATGGGTGGCCACTGATGGAGCATTGAATAC |
| | ACCTAAAGATCATATAGGAACCCGCAATCCGGCCAATAACG |
| | CCGCTATTGTGTTACAATTGCCCCAGGGTACAACCCTTCCTA |
| | AAGGCTTCTACGCCGAGGGGAGCCGGGGCGGAAGCCAAGCC |
| | TCTTCCCGGAGTAGCTCTCGGAGCAGGAACTCCTCAAGGAA |
| | CTCCACTCCTGGGAGCAGCAAAAGAACAAGCCCTGCTCGTA |
| | TGGCCGGGAATGGTGGGGATGCTGCATTGGCCTTGTTACTCC |
| | TGGATAGGCTGAATCAACTTGAATCCAAGATGTCAGGGAAA |
| | GGTCAACAACAACAAGGACAAACCGTAACTAAGAAGAGCG |
| | CCGCCGAAGCCAGCAAGAAACCCAGACAAAAGCGCACTGCT |
| | ACTAAGGCTTATAATGTCACCCAAGCCTTTGGGCGCCGCGGA |
| | CCTGAGCAGACCCAGGGGAACTTCGGGGATCAGGAGCTGAT |
| | CAGGCAAGGCACTGATTATAAGCACTGGCCTCAGATAGCGC |
| | AATTCGCCCCATCAGCTTCCGCATTCTTTGGCATGTCTCGCAT |
| | CGGCATGGAAGTTACTCCTTCTGGAACATGGTTGACGTACAC |
| | CGGGGCAATCAAACTCGATGATAAAGACCCCAACTTTAAGG |
| | ACCAAGTTATCTTGTTGAACAAACACATTGACGCTTATAAAA |
| | CATTCCCACCCACCGAACCTAAGAAGGATAAGAAGAATAAG |
| | GCAGATGAGACCCAGGCTCTCCCCCAAAGGCAAAAGAAGCA |
| | ACAAACTGTAACCCTGCTGCCTGCCGCAGATTTAGACGATTT |
| | CAGCAAGCAACTCCAACAAAGCATGAGCAGAGCTGATTCTA |
| | CCCAAGCCTGA |
| SEQ ID NO: 123 | MSDNGPQNQRNALRITFGGPSDSTGSNQNGGARSKQRRPQGLP |
| Omicron BQ.1 | NNTASWFTALTQHGKEGLKFPRGQGVPINTNSSPDDQIGYYRR |
| Nucleocapsid | ATRRIRGGDGKMKDLSPRWYFYYLGTGPEAGLPYGANKDGII |
| amino acid | WVATDGALNTPKDHIGTRNPANNAAIVLQLPQGTTLPKGFYAE |
| sequence | GSRGGSQASSRSSSRSRNSSRNSTPGSSKRTSPARMAGNGGDAA |
| | LALLLLDRLNQLESKMSGKGQQQQGQTVTKKSAAEASKKPRQ |
| | KRTATKAYNVTQAFGRRGPEQTQGNFGDQELIRQGTDYKHWP |
| | QIAQFAPSASAFFGMSRIGMEVTPSGTWLTYTGAIKLDDKDPNF |
| | KDQVILLNKHIDAYKTFPPTEPKKDKKNKADETQALPQRQKKQ |
| | QTVTLLPAADLDDFSKQLQQSMSRADSTQA |
| SEQ ID NO: 124 | ATGTTTGTGTTCCTCGTACTGCTTCCTCTTGTATCCTCACAAT |
| Omicron | GCGTGAACCTGATAACGCGTACACAATCCTATACCAACTCAT |
| XBB1.5 spike | TTACACGGGGAGTCTACTACCCCGACAAAGTGTTTAGGTCCT |
| protein with 2P | CTGTTTTGCATAGCACGCAAGATCTGTTTCTCCCATTCTTTTC |
| modification | AAATGTAACATGGTTCCACGCCATACACGTGTCCGGAACCA |
| nucleic acid | ATGGCACCAAGCGCTTCGACAATCCAGCTCTCCCTTTTAACG |
| sequence | ATGGTGTATATTTCGCATCAACTGAGAAGAGCAATATAATAC |
| | GCGGCTGGATTTTCGGAACAACTTTAGACTCAAAGACGCAA |
| | TCTCTCCTCATTGTTAATAACGCCACCAACGTGGTCATCAAA |
| | GTCTGTGAATTTCAATTCTGTAATGATCCGTTCTTGGATGTCT |
| | ACCAAAAGAACAATAAATCCTGGATGGAATCTGAGTTTCGG |
| | GTGTATTCCTCAGCTAACAATTGTACTTTCGAGTACGTGTCT |
| | CAGCCTTTCTTCATGGACCTCGAGGGTAAAGAAGGCAATTTC |
| | AAGAACCTTCGCGAGTTCGTCTTCAAGAATATTGACGGGTAT |
| | TTTAAGATTTATTCTAAACACACACCTATTAATCTTGAGCGG |
| | GATCTCCCCCAAGGCTTCAGTGCACTGGAGCCACTTGTCGAC |
| | TTGCCGATAGGCATCAATATTACAAGGTTTCAAACTTTACTG |
| | GCCTTGCATCGGTCATACCTGACACCTGTCGATTCCTCATCC |
| | GGCTGGACGGCTGGCGCGGCTGCCTACTATGTCGGCTACTTA |
| | CAACCTCGCACATTTCTGCTCAAATATAACGAAAACGGTACA |
| | ATTACCGATGCCGTAGACTGTGCCTTAGACCCCCTCTCAGAA |
| | ACTAAGTGCACATTGAAATCCTTCACAGTGGAAAAGGGGAT |
| | TTATCAGACCTCAAATTTTCGGGTTCAGCCAACCGAAAGCAT |
| | TGTCCGGTTCCCAAACATTACTAATCTTTGTCCCTTCCACGA |
| | AGTCTTCAACGCAACCACCTTCGCAAGCGTCTACGCATGGAA |
| | TAGAAAACGAATTTCAAATTGTGTCGCCGATTATAGCGTTAT |
| | CTATAATTTTGCACCGTTCTTTGCATTTAAGTGCTATGGCGTG |
| | AGCCCAACAAAGTTAAACGACCTGTGTTTCACAAATGTATAC |
| | GCTGACTCATTCGTGATAAGGGGCAACGAGGTTAGCCAAAT |
| | CGCCCCGGGACAAACAGGTAACATAGCTGACTATAACTACA |
| | AACTCCCAGACGACTTCACGGGTTGTGTGATCGCATGGAACT |
| | CTAACAAACTCGATAGTAAGCCCTCTGGAAATTATAATTACC |
| | TTTATCGCTTATTCCGTAAATCCAAACTGAAACCTTTTGAAC |
| | GGGACATCAGTACTGAAATTTACCAGGCAGGCAACAAGCCT |
| | TGTAACGGGGTGGCCGGTCCCAATTGTTACTCTCCCCTGCAA |
| | AGCTATGGTTTCCGACCGACGTACGGCGTCGGGCATCAGCCC |
| | TACCGCGTGGTCGTCCTGTCCTTTGAGCTTCTGCACGCACCA |
| | GCCACAGTTTGCGGCCCTAAGAAGTCCACAAACCTGGTAAA |
| | GAACAAATGTGTTAACTTCAACTTTAACGGGCTCACCGGAAC |
| | TGGAGTATTGACTGAGTCCAATAAGAAGTTCTTACCCTTTCA |
| | ACAATTCGGCCGAGACATCGCAGACACAACTGACGCCGTTC |
| | GGGATCCACAAACCTTGGAAATTTTAGATATTACCCCCTGTA |
| | GTTTTGGTGGGGTGTCCGTTATTACACCCGGCACTAACACGT |
| | CTAACCAGGTCGCCGTGCTGTATCAGGGCGTCAATTGTACTG |
| | AGGTACCCGTTGCAATTCATGCCGATCAGCTGACACCAACTT |
| | GGAGAGTATACAGTACCGGCAGCAATGTGTTTCAGACTCGC |
| | GCCGGCTGCCTGATAGGAGCTGAATACGTGAACAATAGTTA |
| | TGAGTGTGATATCCCCATTGGTGCTGGGATCTGTGCCTCATA |
| | CCAGACGCAAACAAAGAGCCATCGTCGCGCACGTTCCGTTG |
| | CAAGCCAATCTATAATCGCATATACCATGTCCCTCGGAGCAG |
| | AAAACTCCGTGGCATATTCAAACAATAGTATCGCGATTCCGA |
| | CAAATTTTACAATATCCGTCACGACCGAGATTTTGCCAGTCA |
| | GCATGACTAAAACATCTGTCGATTGTACTATGTATATATGTG |
| | GTGATAGTACAGAATGCAGTAATCTGTTGCTGCAGTATGGA |
| | AGCTTTTGCACCCAACTCAAGCGGGCTCTTACTGGAATTGCC |
| | GTTGAACAAGATAAGAATACCCAGGAAGTGTTTGCTCAGGT |
| | CAAACAGATCTATAAGACTCCACCAATAAAATACTTTGGTG |
| | GTTTTAATTTCTCCCAAATACTCCCCGATCCCAGCAAGCCGT |
| | CTAAGAGGTCTTTTATCGAAGATCTTCTTTTCAACAAGGTCA |
| | CACTTGCTGATGCTGGATTTATTAAACAATATGGCGATTGTT |
| | TGGGAGACATTGCGGCGCGGGACCTTATATGTGCGCAAAAG |
| | TTCAATGGTCTCACCGTCCTTCCACCCTTATTAACAGATGAG |
| | ATGATCGCCCAATACACTAGCGCATTACTGGCTGGCACTATC |
| | ACCTCTGGGTGGACATTCGGAGCAGGCGCTGCTCTGCAAATC |
| | CCATTTGCAATGCAGATGGCATACAGATTTAATGGCATTGGG |
| | GTCACGCAAAACGTTCTTTATGAAAACCAAAAGTTGATTGCC |
| | AACCAGTTCAATTCCGCGATCGGGAAGATCCAAGATTCACT |
| | GTCAAGTACCGCTTCCGCACTCGGTAAACTCCAAGATGTGGT |
| | CAACCATAATGCACAAGCTCTTAATACCCTGGTAAAACAACT |
| | CTCTTCAAAGTTCGGCGCAATCAGTTCCGTGCTCAATGATAT |
| | TCTTTCCAGGCTCGACCCGCCAGAGGCTGAGGTCCAAATCGA |
| | CAGATTAATAACAGGCCGTTTACAATCACTCCAAACTTACGT |
| | GACACAACAATTAATTAGGGCAGCGGAAATCCGCGCTTCTG |
| | CCAACCTGGCCGCAACAAAGATGTCCGAATGTGTCCTGGGG |
| | CAATCCAAAAGAGTTGACTTTTGTGGCAAGGGTTACCACTTA |
| | ATGTCCTTTCCCCAAAGCGCTCCTCATGGCGTGGTTTTCCTGC |
| | ACGTGACATATGTGCCTGCACAAGAGAAGAACTTTACTACT |
| | GCACCTGCCATATGCCATGATGGCAAGGCTCACTTTCCCCGC |
| | GAGGGCGTGTTCGTTTCCAACGGGACCCACTGGTTCGTCACC |
| | CAACGGAACTTTTATGAGCCACAAATAATTACAACCGATAA |
| | TACCTTTGTATCAGGTAACTGCGATGTGGTCATTGGAATAGT |
| | GAACAATACCGTATATGACCCTCTCCAACCTGAGCTCGATTC |
| | CTTCAAAGAGGAATTAGACAAATACTTTAAGAATCATACTTC |
| | CCCCGATGTCGACCTCGGCGACATCAGTGGTATCAACGCCA |
| | GTGTTGTCAATATCCAAAAGGAAATCGACAGACTCAATGAG |
| | GTAGCAAAGAATCTCAACGAAAGTCTTATAGATCTGCAAGA |
| | ACTGGGGAAGTATGAACAATATATCAAGTGGCCCTGGTATA |
| | TCTGGCTTGGGTTTATCGCTGGACTCATCGCCATTGTGATGG |
| | TTACAATTATGCTCTGTTGTATGACTAGCTGTTGCAGCTGTCT |
| | TAAAGGATGCTGCAGCTGTGGATCTTGCTGTAAGTTCGATGA |
| | GGATGACTCAGAGCCTGTATTGAAGGGCGTGAAACTCCACT |
| | ACACCTGA |
| SEQ ID NO: 125 | MFVFLVLLPLVSSQCVNLITRTQSYTNSFTRGVYYPDKVFRSSV |
| Omicron | LHSTQDLFLPFFSNVTWFHAIHVSGTNGTKRFDNPALPFNDGVY |
| XBB1.5 spike | FASTEKSNIIRGWIFGTTLDSKTQSLLIVNNATNVVIKVCEFQFC |
| protein with 2P | NDPFLDVYQKNNKSWMESEFRVYSSANNCTFEYVSQPFFMDL |
| modification | EGKEGNFKNLREFVFKNIDGYFKIYSKHTPINLERDLPQGFSALE |
| amino acid | PLVDLPIGINITRFQTLLALHRSYLTPVDSSSGWTAGAAAYYVG |
| sequence | YLQPRTFLLKYNENGTITDAVDCALDPLSETKCTLKSFTVEKGI |
| | YQTSNFRVQPTESIVRFPNITNLCPFHEVFNATTFASVYAWNRK |
| | RISNCVADYSVIYNFAPFFAFKCYGVSPTKLNDLCFTNVYADSF |
| | VIRGNEVSQIAPGQTGNIADYNYKLPDDFTGCVIAWNSNKLDS |
| | KPSGNYNYLYRLFRKSKLKPFERDISTEIYQAGNKPCNGVAGPN |
| | CYSPLQSYGFRPTYGVGHQPYRVVVLSFELLHAPATVCGPKKS |
| | TNLVKNKCVNFNFNGLTGTGVLTESNKKFLPFQQFGRDIADTT |
| | DAVRDPQTLEILDITPCSFGGVSVITPGTNTSNQVAVLYQGVNC |
| | TEVPVAIHADQLTPTWRVYSTGSNVFQTRAGCLIGAEYVNNSY |
| | ECDIPIGAGICASYQTQTKSHRRARSVASQSIIAYTMSLGAENSV |
| | AYSNNSIAIPTNFTISVTTEILPVSMTKTSVDCTMYICGDSTECSN |
| | LLLQYGSFCTQLKRALTGIAVEQDKNTQEVFAQVKQIYKTPPIK |
| | YFGGFNFSQILPDPSKPSKRSFIEDLLFNKVTLADAGFIKQYGDC |
| | LGDIAARDLICAQKFNGLTVLPPLLTDEMIAQYTSALLAGTITSG |
| | WTFGAGAALQIPFAMQMAYRENGIGVTQNVLYENQKLIANQF |
| | NSAIGKIQDSLSSTASALGKLQDVVNHNAQALNTLVKQLSSKF |
| | GAISSVLNDILSRLDPPEAEVQIDRLITGRLQSLQTYVTQQLIRA |
| | AEIRASANLAATKMSECVLGQSKRVDFCGKGYHLMSFPQSAPH |
| | GVVFLHVTYVPAQEKNFTTAPAICHDGKAHFPREGVFVSNGTH |
| | WFVTQRNFYEPQIITTDNTFVSGNCDVVIGIVNNTVYDPLQPEL |
| | DSFKEELDKYFKNHTSPDVDLGDISGINASVVNIQKEIDRLNEV |
| | AKNLNESLIDLQELGKYEQYIKWPWYIWLGFIAGLIAIVMVTIM |
| | LCCMTSCCSCLKGCCSCGSCCKFDEDDSEPVLKGVKLHYT |
| SEQ ID NO: 126 | ATGTTCGTTTTCCTCGTGCTGCTCCCCCTCGTCTCTTCACAAT |
| Beta spike | GCGTGAATCTTACGACAAGAACACAATTACCGCCTGCCTAC |
| protein with 2P | ACAAATTCATTTACAAGGGGCGTATACTATCCCGATAAGGTG |
| modification and | TTTCGATCTTCCGTGCTGCATTCAACCCAAGACCTCTTTCTTC |
| furin cleavage | CCTTCTTCTCCAACGTGACTTGGTTCCACGCCATACATGTTA |
| mutation nucleic | GCGGCACAAATGGTACTAAACGCTTCGCCAACCCCGTTCTCC |
| acid sequence | CATTCAATGATGGAGTGTATTTCGCAAGCACCGAAAAGTCTA |
| | ACATTATTCGAGGCTGGATATTTGGAACTACTCTGGACTCCA |
| | TGATTAAAGTCTGTGAGTTCCAATTTTGTAACGATCCATTTCT |
| | AGACACAATCTTTGCTCATTGTTAACAACGCTACTAATGTGG |
| | GGGAGTCTATTATCACAAGAACAACAAATCCTGGATGGAGA |
| | AATACGTATCCCAGCCGTTTCTCATGGACTTGGAAGGGAAAC |
| | GTGAATTTCGGGTATACAGCTCCGCAAACAACTGTACGTTCG |
| | AAGGGAACTTTAAGAATCTCCGTGAATTTGTGTTCAAGAACA |
| | TCGACGGTTATTTCAAAATATATTCTAAACACACGCCCATAA |
| | ACCTTGTCCGAGGACTCCCCCAGGGATTTTCCGCTCTCGAAC |
| | CCTTGGTTGACCTGCCTATAGGCATTAACATCACTAGATTCC |
| | AAACTCTCCACAGATCATACTTAACGCCTGGAGATAGCTCAT |
| | CTGGATGGACAGCCGGGGCAGCAGCATATTATGTCGGGTAC |
| | CTGCAACCGAGAACTTTCCTCTTAAAATACAACGAAAATGG |
| | CACCATTACTGACGCCGTGGATTGTGCCCTTGACCCTTTGAG |
| | CGAAACCAAGTGTACTTTAAAATCCTTCACTGTAGAGAAAG |
| | GCATATACCAAACTTCCAATTTTAGGGTGCAACCAACTGAAT |
| | CAATTGTCAGATTCCCCAATATTACGAACCTGTGTCCGTTTG |
| | GCGAAGTGTTTAACGCCACGCGCTTCGCGTCTGTCTATGCCT |
| | GGAATCGGAAGAGAATAAGCAATTGTGTCGCAGACTACAGT |
| | GTCTTGTACAATAGCGCTTCCTTCAGTACCTTCAAATGTTAT |
| | GGGGTGTCTCCTACGAAACTCAACGATTTATGCTTCACCAAC |
| | GTCTACGCTGACTCCTTTGTGATAAGGGGCGATGAAGTACGA |
| | CAAATAGCGCCAGGACAAACCGGCAACATCGCTGACTACAA |
| | CTATAAGCTGCCAGATGATTTTACAGGGTGTGTCATCGCTTG |
| | GAACTCTAATAACTTGGACTCAAAAGTTGGTGGCAATTACA |
| | ACTATCTCTACAGGTTATTTCGCAAAAGCAACCTCAAACCAT |
| | TTGAGAGAGATATCTCTACCGAAATCTACCAGGCCGGCAGC |
| | ACCCCTTGTAACGGTGTGAAGGGTTTCAATTGTTATTTTCCTC |
| | TCCAATCCTATGGCTTTCAACCAACTTATGGTGTCGGTTACC |
| | AACCATATAGAGTAGTTGTGTTGTCCTTTGAGTTACTCCATG |
| | CCCCTGCCACTGTGTGTGGACCTAAGAAATCTACCAACTTAG |
| | TTAAGAACAAATGCGTAAACTTTAACTTTAACGGATTAACCG |
| | GAACCGGGGTGTTGACTGAAAGTAACAAGAAATTCTTGCCT |
| | TTCCAACAATTTGGCAGAGACATCGCAGATACCACAGATGC |
| | CGTTCGTGATCCCCAAACACTCGAAATCTTGGACATAACACC |
| | CTGTTCTTTTGGTGGAGTAAGCGTTATAACTCCAGGCACTAA |
| | CACTTCCAACCAAGTCGCAGTACTCTATCAAGGGGTTAACTG |
| | CACAGAGGTACCAGTGGCCATCCATGCAGACCAACTCACCC |
| | CCACATGGAGAGTTTACAGCACGGGGTCCAATGTCTTTCAAA |
| | CGCGAGCGGGCTGTCTGATAGGAGCAGAACACGTCAATAAC |
| | TCATACGAGTGTGATATCCCCATTGGAGCAGGGATTTGCGCC |
| | TCTTATCAAACGCAAACCAACTCACCTGGTTCCGCATCCAGC |
| | GTCGCGAGCCAATCAATTATAGCCTATACAATGTCCTTAGGC |
| | GTTGAAAACAGCGTCGCTTACTCAAATAACAGCATAGCCAT |
| | ACCCACAAACTTCACAATTAGCGTTACCACTGAAATACTCCC |
| | AGTTAGCATGACAAAGACTTCCGTTGATTGCACCATGTACAT |
| | CTGTGGCGACTCAACAGAGTGCAGCAATCTGCTGCTCCAATA |
| | CGGGTCTTTCTGTACCCAGCTCAATAGGGCACTGACTGGTAT |
| | AGCAGTGGAACAAGACAAGAATACTCAGGAAGTATTCGCCC |
| | AGGTGAAACAGATTTACAAAACTCCCCCTATCAAAGATTTCG |
| | GCGGCTTTAACTTCTCCCAAATCCTGCCCGATCCTTCCAAAC |
| | CGTCCAAAAGGTCCTTTATTGAAGACCTGCTGTTTAACAAAG |
| | TGACCCTTGCAGATGCCGGATTCATTAAACAATATGGGGACT |
| | GTCTCGGGGATATAGCCGCGAGGGATTTAATCTGTGCTCAAA |
| | AGTTCAATGGCCTCACCGTTCTCCCACCCCTTCTTACCGACG |
| | AAATGATTGCCCAATATACTAGTGCGCTCCTGGCTGGTACGA |
| | TAACTAGCGGCTGGACTTTCGGCGCCGGTGCGGCCCTGCAA |
| | ATACCCTTTGCTATGCAAATGGCGTACCGCTTTAATGGCATC |
| | GGGGTTACACAAAATGTACTTTACGAGAATCAAAAGCTCAT |
| | CGCAAACCAATTTAATTCAGCAATCGGAAAGATCCAAGACT |
| | CCTTGAGCAGCACCGCGTCTGCCTTGGGCAAACTCCAGGAC |
| | GTTGTGAACCAAAATGCGCAGGCACTGAACACTCTTGTTAA |
| | ACAATTGAGTAGCAACTTTGGCGCAATTAGCTCTGTACTTAA |
| | TGATATTCTGAGCCGGCTGGACCCACCAGAGGCCGAAGTCC |
| | AAATAGACCGTCTGATTACGGGACGGCTTCAATCCTTGCAGA |
| | CCTACGTTACCCAACAACTTATCAGGGCTGCCGAGATAAGA |
| | GCAAGTGCCAACCTTGCTGCCACCAAGATGTCCGAGTGTGTA |
| | CTCGGCCAATCCAAGCGCGTCGACTTTTGTGGGAAGGGATAT |
| | CACTTGATGTCTTTCCCTCAATCTGCCCCTCACGGCGTGGTGT |
| | TCCTGCATGTGACCTACGTGCCAGCGCAAGAAAAGAACTTT |
| | ACCACTGCTCCAGCAATTTGTCACGACGGCAAAGCACATTTT |
| | CCCAGGGAAGGAGTGTTTGTTTCTAATGGAACGCACTGGTTC |
| | GTAACACAAAGAAATTTCTATGAACCCCAAATCATTACTACT |
| | GATAACACCTTCGTTTCTGGTAACTGTGACGTAGTTATCGGA |
| | ATCGTAAACAACACTGTATATGACCCTCTGCAGCCAGAGCTC |
| | GACAGTTTTAAGGAAGAGTTGGATAAGTATTTTAAGAATCAT |
| | ACCTCTCCTGACGTCGACCTCGGCGACATTTCCGGCATAAAT |
| | GCCTCAGTCGTGAATATCCAGAAAGAAATAGACCGCTTGAA |
| | CGAGGTTGCGAAGAATTTGAACGAGTCCTTGATAGACCTGC |
| | AAGAGCTGGGAAAGTATGAGCAATACATCAAGTGGCCGTGG |
| | TATATATGGCTCGGGTTTATTGCTGGTTTAATCGCTATCGTG |
| | ATGGTGACAATAATGCTTTGTTGTATGACATCATGTTGCAGT |
| | TGTCTCAAAGGGTGTTGCAGTTGCGGCTCCTGCTGCAAGTTC |
| | GATGAGGACGATTCTGAACCAGTGCTCAAAGGGGTTAAGCT |
| | TCATTATACGTAG |
| SEQ ID NO: 127 | MFVFLVLLPLVSSQCVNLTTRTQLPPAYTNSFTRGVYYPDKVFR |
| Beta spike | SSVLHSTQDLFLPFFSNVTWFHAIHVSGTNGTKRFANPVLPEND |
| protein with 2P | GVYFASTEKSNIIRGWIFGTTLDSKTQSLLIVNNATNVVIKVCEF |
| modification and | QFCNDPFLGVYYHKNNKSWMESEFRVYSSANNCTFEYVSQPFL |
| furin cleavage | MDLEGKQGNFKNLREFVFKNIDGYFKIYSKHTPINLVRGLPQGF |
| mutation amino | SALEPLVDLPIGINITRFQTLHRSYLTPGDSSSGWTAGAAAYYV |
| acid sequence | GYLQPRTFLLKYNENGTITDAVDCALDPLSETKCTLKSFTVEKG |
| | IYQTSNFRVQPTESIVRFPNITNLCPFGEVENATRFASVYAWNRK |
| | RISNCVADYSVLYNSASFSTFKCYGVSPTKLNDLCFTNVYADSF |
| | VIRGDEVRQIAPGQTGNIADYNYKLPDDFTGCVIAWNSNNLDS |
| | KVGGNYNYLYRLFRKSNLKPFERDISTEIYQAGSTPCNGVKGFN |
| | CYFPLQSYGFQPTYGVGYQPYRVVVLSFELLHAPATVCGPKKS |
| | TNLVKNKCVNFNFNGLTGTGVLTESNKKFLPFQQFGRDIADTT |
| | DAVRDPQTLEILDITPCSFGGVSVITPGTNTSNQVAVLYQGVNC |
| | TEVPVAIHADQLTPTWRVYSTGSNVFQTRAGCLIGAEHVNNSY |
| | ECDIPIGAGICASYQTQTNSPGSASSVASQSIIAYTMSLGVENSV |
| | AYSNNSIAIPTNFTISVTTEILPVSMTKTSVDCTMYICGDSTECSN |
| | LLLQYGSFCTQLNRALTGIAVEQDKNTQEVFAQVKQIYKTPPIK |
| | DFGGFNFSQILPDPSKPSKRSFIEDLLFNKVTLADAGFIKQYGDC |
| | LGDIAARDLICAQKFNGLTVLPPLLTDEMIAQYTSALLAGTITSG |
| | WTFGAGAALQIPFAMQMAYRENGIGVTQNVLYENQKLIANQF |
| | NSAIGKIQDSLSSTASALGKLQDVVNQNAQALNTLVKQLSSNF |
| | GAISSVLNDILSRLDPPEAEVQIDRLITGRLQSLQTYVTQQLIRA |
| | AEIRASANLAATKMSECVLGQSKRVDFCGKGYHLMSFPQSAPH |
| | GVVFLHVTYVPAQEKNFTTAPAICHDGKAHFPREGVFVSNGTH |
| | WFVTQRNFYEPQIITTDNTFVSGNCDVVIGIVNNTVYDPLQPEL |
| | DSFKEELDKYFKNHTSPDVDLGDISGINASVVNIQKEIDRLNEV |
| | AKNLNESLIDLQELGKYEQYIKWPWYIWLGFIAGLIAIVMVTIM |
| | LCCMTSCCSCLKGCCSCGSCCKFDEDDSEPVLKGVKLHYT |
| SEQ ID NO: 128 | GTAAGTACCGCCTATAGACTCTATAGGCACACCCCTTTGGCT |
| CMV intron A | CTTATGCATGCTATACTGTTTTTGGCTTGGGGCCTATACACCC |
| | CCGCTCCTTATGCTATAGGTGATGGTATAGCTTAGCCTATAG |
| | GTGTGGGTTATTGACCATTATTGACCACTCCCCTATTGGTGA |
| | CGATACTTTCCATTACTAATCCATAACATGGCTCTTTGCCAC |
| | AACTATCTCTATTGGCTATATGCCAATACTCTGTCCTTCAGA |
| | GACTGACACGGACTCTGTATTTTTACAGGATGGGGTCCCATT |
| | TATTATTTACAAATTCACATATACAACAACGCCGTCCCCCGT |
| | GCCCGCAGTTTTTATTAAACATAGCGTGGGATCTCCACGCGA |
| | ATCTCGGGTACGTGTTCCGGACATGGGCTCTTCTCCGGTAGC |
| | GGCGGAGCTTCCACATCCGAGCCCTGGTCCCATGCCTCCAGC |
| | GGCTCATGGTCGCTCGGCAGCTCCTTGCTCCTAACAGTGGAG |
| | GCCAGACTTAGGCACAGCACAATGCCCACCACCACCAGTGT |
| | GCCGCACAAGGCCGTGGCGGTAGGGTATGTGTCTGAAAATG |
| | AGCTCGGAGATTGGGCTCGCACCGTGACGCAGATGGAAGAC |
| | TTAAGGCAGCGGCAGAAGAAGATGCAGGCAGCTGAGTTGTT |
| | GTATTCTGATAAGAGTCAGAGGTAACTCCCGTTGCGGTGCTG |
| | TTAACGGTGGAGGGCAGTGTAGTCTGAGCAGTACTCGTTGCT |
| | GCCGCGCGCGCCACCAGACATAATAGCTGACAGACTAACAG |
| | ACTGTTCCTTTCCATGGGTCTTTTCTGCAG |
| SEQ ID NO: 129 | ATGGATTGGACCTGGATCCTGTTCCTGGTTGCAGCAGCTACC |
| IgE leader | CGTGTGCACTCT |
| sequence nucleic | |
| acid sequence | |
| SEQ ID NO: 130 | MDWTWILFLVAAATRVHS |
| IgE leader | |
| sequence amino | |
| acid sequence | |
| SEQ ID NO: 131 | GCGCAGCACCATGGCCTGAAATAACCTCTAAAGAGGAACTT |
| SV40 promoter | GGTTAGGTACCTTCTGAGGCGGAAAGAACCAGCTGTGGAAT |
| | GTGTGTCAGTTAGGGTGTGGAAAGTCCCCAGGCTCCCCAGC |
| | AGGCAGAAGTATGCAAAGCATGCATCTCAATTAGTCAGCAA |
| | CCAGGTGTGGAAAGTCCCCAGGCTCCCCAGCAGGCAGAAGT |
| | ATGCAAAGCATGCATCTCAATTAGTCAGCAACCATAGTCCCG |
| | CCCCTAACTCCGCCCATCCCGCCCCTAACTCCGCCCAGTTCC |
| | GCCCATTCTCCGCCCCATGGCTGACTAATTTTTTTTATTTATG |
| | CAGAGGCCGAGGCCGCCTCGGCCTCTGAGCTATTCCAGAAG |
| | TAGTGAGGAGGCTTTTTTGGAGGCCTAGGCTTTTGCAAAAAG |
| | CTT |
| SEQ ID NO: 132 | atggcagattccaacggtactattaccgttgaagagcttaaaaagctccttgaacaatggaacctagta |
| SARS-CoV-2 M | ataggtttcctattccttacatggatttgtcttctacaatttgcctatgccaacaggaataggtttttgta |
| Protein (nucleic | tataattaagttaattttcctctggctgttatggccagtaactttagcttgttttgtgcttgctgctgttt |
| acid sequence) | acagaataaattggatcaccggtggaattgcttcgcaatggcttgtcttgtaggcttgatgtggctcagct |
| | acttcattgcttctttcagactgtttgcgcgtacgcgttccatgtggtcattcaatccagaaactaacatt |
| | cttctcaacgtgccactccatggcactattctgaccagaccgcttctagaaagtgaactcgtaatcggagc |
| | tgtgatccttcgtggacatcttcgtattgctggacaccatctaggacgctgtgacatcaaggacctgccta |
| | aagaaatcactgttgctacatcacgaacgctttcttattacaaattgggagcttcgcagcgtgtagcaggt |
| | gactcaggttttgctgcatacagtcgctacaggattggcaactataaattaaacacagaccattccagtagc |
| | agtgacaatattgctttgcttgtacagtaa |

What is claimed is:

1. A composition comprising:
(a) a polynucleotide comprising an antigen nucleic acid which encodes a pathogen protein or an antigenic fragment thereof, wherein the antigen nucleic acid is operably linked to a promoter;
(b) a delivery component selected from the group consisting of a cationic polymer, a poly-inosinic-polycytidylic acid, a poloxamer, or derivative thereof, and, optionally,
(c) an adjuvant comprising an aluminum or aluminum-salt based adjuvant, a stimulator of interferon genes (STING) agonist, an unmethylated cytosine-guanine dinucleotide-containing oligodeoxynucleotide (CpG), a M59 oil-in-water emulsion of squalene oil, an AS03 α-tocopherol, squalene, and polysorbate 80 in an oil-in-water emulsion, or any combination thereof.

2. The composition of claim 1, wherein the aluminum or aluminum-salt based adjuvant is selected from the group consisting of an aluminum phosphate, aluminum carbonate, an aluminum hydroxide, aluminum oxyhydroxide, an aluminum bicarbonate, a potassium aluminum sulfate [KAl(SO₄)₂], an aluminum hydroxyphosphate, an aluminum hydroxyphosphate sulfate, an aluminum chloride, an aluminum silicate, and any combination thereof.

3. The composition of claim 1 or 2, wherein the poloxamer is crown poloxamer and the adjuvant comprises an aluminum or aluminum-salt based adjuvant, a STING agonist, an unmethylated cytosine-guanine dinucleotide-containing oligodeoxynucleotide (CpG), a M59 oil-in-water emulsion of squalene oil, an AS03 α-tocopherol, squalene, and polysorbate 80 in an oil-in-water emulsion, or any combination thereof.

4. The compositions of any one of claims 1-3, comprising (c) an adjuvant comprising an aluminum or aluminum-salt based adjuvant, a stimulator of interferon genes (STING) agonist, an unmethylated cytosine-guanine dinucleotide-containing oligodeoxynucleotide (CpG), a M59 oil-in-water emulsion of squalene oil, an AS03 α-tocopherol, squalene, and polysorbate 80 in an oil-in-water emulsion, or any combination thereof.

5. The composition of any one of claims 1-4, wherein the composition comprises the aluminum salt-based adjuvant at 0.005%-0.5% (w/v).

6. The composition of any one of claims 1-5, wherein the STING agonist is selected from a group consisting of a cyclic di-nucleotides, a non-cyclic di-nucleotide small molecule, an amidobenzimidazole (ABZI), a flavonoid, a nanovaccine, an antibody drug conjugate, a bacterial vector, and an ENPP1 inhibitor; and
wherein the STING agonist is present in the composition from about 0.001 to about 1 mg/ml.

7. The composition of any one of claims 1-6, wherein the STING agonist is cyclic guanosine monophosphate (cGMP), cyclic adenosine monophosphate (cAMP), cyclic guanosine monophosphate-adenosine monophosphate (cGAMP), an amidobenzimidazole, or flavonoid.

8. The composition of any one of claims 1-7, wherein the STING agonist is cGMP.

9. The composition of any one of claims 1-8, wherein the pathogen protein is selected from the group consisting of a viral protein, a bacterial protein, a parasite protein, and any antigenic fragments thereof.

10. The composition of any one of claims 1-9, wherein the polynucleotide comprises a single nucleic acid which encodes a single pathogen protein or antigenic fragment thereof.

11. The composition of any one of claims 1-9, wherein the polynucleotide comprises an additional antigen nucleic acid, which encodes a second pathogen protein or an antigenic fragment thereof.

12. The composition of claim 11, wherein the second pathogen protein is selected from the group consisting of a viral protein, a bacterial protein, a parasite protein, and any antigenic fragments thereof.

13. The composition of any one of claims 1-12, wherein the pathogen protein and/or the second pathogen protein is/are selected from the group consisting of a Yersinia pestis antigen, a Mycobacterium tuberculosis antigen, a Meningococcus antigen, an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a Rabies virus antigen, a Cytomegalovirus antigen, a Yellow fever virus antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a Delta variant SARS-CoV antigen, an Omicron variant SARS-CoV antigen, a orthopoxvirus antigen, a monkeypox antigen, a vaccinia antigen, a smallpox antigen, a Epstein bar virus antigen, a nipha virus antigen, a varicella-zoster virus antigen, a Clostridioides difficile antigen, a Streptococcus pneumonia antigen, a Neisseria meningitides antigen, a Toxoplasma gondii antigen, a Plasmodium falciparum antigen, antigenic fragments thereof, and any composition thereof.

14. The composition of claim 13, wherein the pathogen protein and/or the second pathogen protein is/are selected from the group consisting of: a Yersinia pestis F1-Ag, a Yersinia pestis V-Ag, a Mycobacterium tuberculosis Apa antigen, a Mycobacterium tuberculosis HP65 antigen, a Mycobacterium tuberculosis rAg85A antigen, an E71 VP1 antigen, a GST-tagged E71-VP1 antigen, a Cox protein antigen, a GST-tagged Cox protein antigen, an HSV-1 envelope antigen, an HSV-2 envelope antigen, an HSV-2 gB2 antigen, an HSV-2 gC2 antigen, an HSV-2 gD2 antigen, an HSV-2 gE2 antigen, an HIV Env antigen, an HIV Gag antigen, an HIV Nef antigen, an HIV Pol antigen, an HPV minor capsid protein L2 antigen, a human papillomavirus type 16 Regulatory protein E2 antigen, a human papillomavirus type 16 Protein E6 antigen, a human papillomavirus type 16 Protein E7 antigen, a human papillomavirus type 18 Regulatory protein E2 antigen, a human papillomavirus type 18 Protein E6 antigen, a human papillomavirus type 18 Protein E7 antigen, a human papillomavirus type 6a Regulatory protein E2 antigen, a human papillomavirus type 6a Protein E6 antigen, a human papillomavirus type 6a Protein E7 antigen, a human papillomavirus 11 Regulatory protein E2 antigen, a human papillomavirus 11 Protein E6 antigen, a human papillomavirus 11 Protein E7 antigen, an HCV NS3 antigen, a hepatitis C virus genotype 1a Genome polyprotein antigen, a hepatitis C virus genotype 1b Genome polyprotein antigen, a hepatitis C virus genotype 2a Genome polyprotein antigen, a hepatitis C virus genotype 3a Genome polyprotein antigen, a RSV F antigen, a RSV G antigen, a Dengue virus E protein antigen, a Dengue virus EDIII antigen, a Dengue virus NS1 antigen, a Dengue virus DEN-80E antigen, an Ebola virus GB antigen, an Ebola virus VP24 antigen, an Ebola virus VP40 antigen, an Ebola virus NP antigen, an Ebola virus VP30 antigen, an Ebola virus VP35 antigen, a Zika virus envelope domain III antigen, a Zika virus CKD antigen, a Chikungunya virus E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), a measles virus hemagglutinin protein MV-H antigen, a measles virus fusion protein MV-F antigen, a MERS-CoV S protein antigen, an antigen from the receptor-binding domain of the MERS-CoV S protein, an antigen from the membrane fusion domain of the MERS-CoV S protein, a SARS-CoV S protein antigen, an antigen from the receptor binding domain of the SARS-CoV S protein, an antigen from the membrane fusion domain of the SARS-CoV S protein, a SARS-CoV E protein antigen, a SARS-CoV M protein antigen, a SARS-CoV N protein antigen, a monkeypox A35R protein antigen, a monkeypox H3L protein antigen, a monkeypox L1R protein antigen a Clostridioides difficile 630 spore coat protein: peroxiredoxin/chitinase antigen, a Clostridioides difficile 630 flagellin C antigen, a Clostridioides difficile Surface layer protein A (Fragment) antigen, an Epstein-Barr virus (strain B95-8) nuclear antigen 1 antigen, an Epstein-Barr virus (strain B95-8) Envelope glycoprotein B antigen, an Epstein-Barr virus (strain B95-8) Envelope glycoprotein H antigen, an Epstein-Barr virus (strain B95-8) Envelope glycoprotein GP350 antigen, an Epstein-Barr virus (strain B95-8) Latent membrane protein 1 antigen, an Epstein-Barr virus (strain B95-8) Latent membrane protein 2 antigen, a Neisseria meningitides Factor H-binding protein antigen, a Neisseria meningitidis serogroup B Neisseria adhesin A antigen, a Neisseria meningitidis Neisserial heparin binding antigen antigen, a Vaccinia virus (strain Western Reserve) Protein A27 antigen, a Vaccinia virus (strain Western Reserve) EEV membrane phosphoglycoprotein antigen, a Vaccinia virus B5R (Fragment) antigen, a Vaccinia virus Envelope protein H₃ antigen, a Vaccinia virus (strain Western Reserve) IMV membrane protein antigen, a Nipah virus Fusion glycoprotein FO antigen, a Nipah virus Glycoprotein G antigen, a Varicella-zoster virus (strain Dumas) Envelope glycoprotein E antigen, a Toxoplasma gondii MIC8 antigen, a Plasmodium falciparum SERA5 polypeptide antigen, a Plasmodium falciparum circumsporozite protein antigen, antigenic fragments thereof, and any combination thereof.

15. The composition of any one of claims 1-14, wherein the pathogen protein and/or the second pathogen protein is a SARS-CoV-2 antigen or an antigenic fragment thereof.

16. The composition of claim 15, wherein the pathogen protein is a SARS-CoV-2 protein or an antigenic fragment thereof selected from the group consisting of: a SARS CoV-2 spike (S) protein, a Delta variant SARS CoV-2 spike (S) protein, an Omicron variant SARS CoV-2 spike (S) protein, a SARS-CoV-2 membrane (M) protein, a Delta variant SARS-CoV-2 membrane (M) protein, an Omicron variant SARS-CoV-2 membrane (M) protein, a SARS-CoV-2 envelope (E) protein, a Delta variant SARS-CoV-2 envelope (E) protein, an Omicron variant SARS-CoV-2 envelope (E) protein, a SARS-CoV-2 nucleocapsid (N) protein, a Delta variant SARS-CoV-2 nucleocapsid (N) protein, an Omicron variant SARS-CoV-2 nucleocapsid (N) protein, or an antigenic fragment thereof, and wherein the second pathogen protein is a SARS-CoV-2 protein or an antigenic fragment thereof selected from the group consisting of: a SARS CoV-2 spike (S) protein, a Delta variant SARS CoV-2 spike (S) protein, an Omicron variant SARS CoV-2 spike (S) protein a SARS-CoV-2 membrane (M) protein, a Delta variant SARS-CoV-2 membrane (M) protein, an Omicron variant SARS-CoV-2 membrane (M) protein a SARS-CoV-2 envelope (E) protein, a Delta variant SARS-CoV-2 envelope (E) protein, an Omicron variant SARS-CoV-2 envelope (E) protein a SARS-CoV-2 nucleocapsid (N) protein, a Delta variant SARS-CoV-2 nucleocapsid (N) protein, an Omicron variant SARS-CoV-2 nucleocapsid (N) protein, or an antigenic fragment thereof.

17. The composition of claim 16, wherein the second pathogen protein is selected from the group consisting of an influenza virus hemagglutinin (HA) antigen, an influenza virus neuraminidase (NA) antigen, an influenza virus matrix-1 (M1) protein antigen, an influenza virus matrix-2 (M2) protein antigen, an influenza RNA polymerase subunit PB1 antigen, an influenza RNA polymerase subunit PB2 antigen, an influenza RNA polymerase subunit PA antigen, an influenza non-structural protein 1 (NS1) antigen, an influenza non-structural protein 2 (NS2) protein antigen, antigenic fragments thereof, and any combination thereof.

18. The composition of any one of claims 1-12, wherein the antigen nucleic acid of encodes a SARS CoV-2 S protein or an antigenic fragment thereof.

19. The composition of any one of claims 11-18, wherein the second pathogen protein or antigenic fragment thereof that is selected from the group consisting of: a SARS-CoV-2 M protein or an antigenic fragment thereof, a SARS-CoV-2 E protein or an antigenic fragment thereof, a SARS-CoV-2 N protein or an antigenic fragment thereof, and any combination thereof.

20. The composition of claim 19, wherein the pathogen protein is selected from a SARS-CoV-2 protein or an antigenic fragment thereof selected from the group consisting of: a SARS CoV-2 spike (S) protein, a SARS-CoV-2 membrane (M) protein, a SARS-CoV-2 envelope (E) protein, a SARS-CoV-2 nucleocapsid (N) protein, or an antigenic fragment thereof, and wherein the second pathogen protein is selected from a SARS-CoV-2 protein or an antigenic fragment thereof selected from the group consisting of: a SARS CoV-2 spike (S) protein, a SARS-CoV-2 membrane (M) protein, a SARS-CoV-2 envelope (E) protein, a SARS-CoV-2 nucleocapsid (N) protein, or an antigenic fragment thereof.

21. The composition of claim 20, wherein the pathogen protein is a SARS-CoV-2 S protein or an antigenic fragment thereof, and wherein the second pathogen protein is a SARS-CoV-2 S protein or an antigenic fragment thereof, and wherein pathogen proteins are derived from different strains of SARS-CoV-2.

22. The composition of any one of claims 11-21, wherein the additional antigen nucleic acid is operably linked to the promoter through an internal ribosome entry site (IRES) sequence.

23. The composition of any one of claims 11-21, wherein the additional antigen nucleic acid is operably linked a second promoter.

24. The composition of any one of claims 1-23, wherein the polynucleotide further comprises a second additional antigen nucleic acid, which encodes a third pathogen protein or an antigenic fragment thereof.

25. The composition of any one of claims 1-24, wherein the second additional antigen nucleic acid is operably linked to a third promoter.

26. The composition of any one of claims 1-25, wherein the promoter and/or the second promoter and/or the third promoter is selected from the group consisting of: a cytomegalovirus (CMV) promoter, a Rouse sarcoma virus (RSV) promoter, a Moloney murine leukemia virus (Mo-MuLV) long terminal repeat (LTR) promoter, a human ubiquitin C promoter, a mammalian elongation factor 1 (EF1) promoter, a human elongation factor 1α/Human T cell Leukemia Virus Type 1 Long Terminal Repeat (hEF1/HTLV) promoter, a cytokeratin 18 (CK18) promoter, a cytokeratin 19 (CK19) promoter, a simian virus 40 (SV40) promoter, a murine U6 promoter, a skeletal α-actin promoter, a β-actin promoter, a murine phosphoglycerate kinase 1 (PGK1) promoter, a human PGK1 promoter, a CBA promoter, a CAG promoter, and any combination thereof.

27. The composition of any one of claims 1-26, wherein the antigen nucleic acid encodes a polypeptide comprising the amino acid sequence of SEQ ID NO: 2, SEQ ID NO: 4, SEQ ID NO: 115, SEQ ID NO: 117, SEQ ID NO: 119, SEQ ID NO: 121, SEQ ID NO: 125, or SEQ ID NO: 127.

28. The composition of any one of claims 1-27, wherein the antigen nucleic acid comprises a nucleic acid sequence having at least 70%, at least 75%, at least 80%, at least 85%, at least 90%, at least 95%, at least 99%, or 100% sequence identity to SEQ ID NO: 1, SEQ ID NO: 3, SEQ ID NO: 114, SEQ ID NO: 116, SEQ ID NO: 118, SEQ ID NO: 120, SEQ ID NO: 124, or SEQ ID NO: 126.

29. The composition of any one of claims 1-26, wherein the antigen nucleic acid encodes the receptor binding domain (RBD) of the SARS-Cov-2 S protein or an antigenic fragment thereof.

30. The composition of any one of claims 1-26, wherein the antigen nucleic acid encodes a polypeptide comprising the amino acid sequence of SEQ ID NO: 6.

31. The composition of any one of claims 1-26, wherein the antigen nucleic acid comprises a nucleic acid sequence having at least 70%, at least 75%, at least 80%, at least 85%, at least 90%, at least 95%, at least 99%, or 100% sequence identity to SEQ ID NO: 5.

32. The composition of any one of claims 1-31, wherein the antigen nucleic acid of the polynucleotide encodes the S1 subunit of the SARS-Cov-2 S protein or an antigenic fragment thereof.

33. The composition of any one of claims 1-26, wherein the antigen nucleic acid encodes a polypeptide comprising the amino acid sequence of SEQ ID NO: 40.

35. The composition of any one of claims 1-26, wherein the antigen nucleic acid comprises a nucleic acid sequence having at least 70%, at least 75%, at least 80%, at least 85%, at least 90%, at least 95%, at least 99%, or 100% sequence identity to SEQ ID NO: 39.

36. The composition of any one of claims 11-33, wherein the first and/or second additional antigen nucleic acid encodes a polypeptide comprising the amino acid sequence of SEQ ID NO: 8, SEQ ID NO: 10 SEQ ID NO: 12, SEQ ID NO: 14, SEQ ID NO: 16, SEQ ID NO: 18, or SEQ ID NO: 20.

37. The composition of any one of claims 11-33, wherein the first and/or second additional antigen nucleic acid comprises a nucleic acid sequence having at least 70%, at least 75%, at least 80%, at least 85%, at least 90%, at least 95%, at least 99%, or 100% sequence identity to SEQ ID NO: 7, SEQ ID NO: 9, SEQ ID NO: 11, SEQ ID NO: 13, SEQ ID NO: 15, SEQ ID NO: 17, SEQ ID NO: 19, or SEQ ID NO: 131.

38. The composition of any one of claims 11-33, wherein the first and/or second additional antigen nucleic acid encodes a polypeptide comprising the amino acid sequence of SEQ ID NO: 22, SEQ ID NO: 24, or SEQ ID NO: 26.

39. The composition of any one of claims 11-33, wherein the first and/or second additional antigen nucleic acid comprises a nucleic acid sequence having at least 70%, at least 75%, at least 80%, at least 85%, at least 90%, at least 95%, at least 99%, or 100% sequence identity to SEQ ID NO: 21, SEQ ID NO: 23, or SEQ ID NO: 25.

40. The composition of any one of claims 11-33, wherein the first and/or second additional antigen nucleic acid encodes a polypeptide comprising the amino acid sequence of SEQ ID NO: 28 or SEQ ID NO: 123.

41. The composition of any one of claims 11-33, wherein the first and/or second additional antigen nucleic acid comprises a nucleic acid sequence having at least 70%, at least 75%, at least 80%, at least 85%, at least 90%, at least 95%, at least 99%, or 100% sequence identity to SEQ ID NO: 27 or SEQ ID NO: 122.

42. The composition of any one of claims 11-33, wherein the first and/or second additional antigen nucleic acid encodes a polypeptide comprising the amino acid sequence of SEQ ID NO: 67, SEQ ID NO: 69, SEQ ID NO: 71, SEQ ID NO: 73, SEQ ID NO: 74, SEQ ID NO: 75, SEQ ID NO: 76, SEQ ID NO: 77 SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, or SEQ ID NO: 113.

43. The composition of any one of claims 11-33, wherein the first and/or second additional antigen nucleic acid comprises a nucleic acid sequence having at least 70%, at least 75%, at least 80%, at least 85%, at least 90%, at least 95%, at least 99%, or 100% sequence identity to SEQ ID NO: 66, SEQ ID NO: 68, SEQ ID NO: 70, or SEQ ID NO; 72.

44. The composition of any one of claims 1-33, further comprising a nucleic acid sequence encoding one or more immune modifier proteins.

45. The composition of claim 44, wherein the one or more immune modifier proteins is a cytokine or a chemokine.

46. The composition of any of claims 44-45, wherein the one or more immune modifier proteins is selected from the group consisting of: IL-2, IL-12 p35, IL-12 p40, IL-12 p70, IL-15, IL-18, TNFα, GM-CSF, IFN-α, IFN-β, MHC I, MHC II, HLA-DR, CD80, CD86, and any combination thereof.

47. The composition of any of claims 44-46, wherein the nucleic acid sequence encoding the one or more immune modifier proteins is operably linked to a promoter.

48. The composition of any of claims 44-47, wherein the promoter is selected from the group consisting of: a cytomegalovirus (CMV) promoter, a Rouse sarcoma virus (RSV) promoter, a Moloney murine leukemia virus (Mo-MuLV) long terminal repeat (LTR) promoter, a human ubiquitin C promoter, a mammalian elongation factor 1 (EF1) promoter, a human elongation factor 1α/Human T cell Leukemia Virus Type 1 Long Terminal Repeat (hEF1/HTLV) promoter, a cytokeratin 18 (CK18) promoter, a cytokeratin 19 (CK19) promoter, a simian virus 40 (SV40) promoter, a murine U6 promoter, a skeletal α-actin promoter, a β-actin promoter, a murine phosphoglycerate kinase 1 (PGK1) promoter, a human PGK1 promoter, a CBA promoter, a CAG promoter, and any combination thereof.

49. The composition of any one of claims 1-48, wherein the polynucleotide further comprises one or more post-transcriptional regulatory elements.

50. The composition of claim 49, wherein the post-transcriptional regulatory element is a wood chuck hepatitis virus post-transcriptional regulatory element (WPRE).

51. The composition of any one of claims 1-45, wherein the polynucleotide further comprises at least one 3′ UTR poly(a) tail sequence operably linked to the antigen nucleic acid, the second antigen nucleic acid, or any combination thereof.

52. The composition of claim 51, wherein the 3′ UTR poly(a) tail sequence is a 3′ UTR SV40 poly(a) tail sequence, a 3′ UTR bovine growth hormone (bGH) poly(A) sequence, a 3′ UTR actin poly(A) tail sequence, a 3′ UTR hemoglobin poly(A) sequence, or combinations thereof.

53. The composition of any one of claims 1-52, wherein the polynucleotide further comprises an enhancer sequence.

54. The composition of claim 53, wherein the enhancer sequence comprises a human actin enhancer sequence, a human myosin enhancer sequence, a human hemoglobin enhancer sequence, a human muscle creatine enhancer sequence, a viral enhancer sequence, a polynucleotide function enhancer sequence, or any combination thereof.

55. The composition of claim 53 or 54, wherein the enhancer sequence comprises a CMV intronic sequence, a β-actin intronic sequence, or the combination thereof.

56. The composition of claim 55, wherein the enhancer sequence is a CMV intronic sequence.

57. The composition of claim 55 or 56, wherein the enhancer sequence is a CMV intronic sequence, a SV40 enhancer sequence, a β-actin intronic sequence, or combinations thereof.

58. The composition of any one of claims 1-57, wherein the polynucleotide is an expression vector.

59. The composition of claim 58, wherein the expression vector is a DNA plasmid.

60. The composition of claim 59, wherein the DNA plasmid vector comprises the elements of a vector selected from the group consisting of pVac 1, pVac 2, pVac 3, pVac 4, pVac 5, pVac 6, pVac 7, pVac 8 pVac 9, pVac 10 pVac 11, pVac 12, pVac 13, pVac 14, pVac 15 pVac 16, pVac 17, pVac 18, pVac 19, pVac 20, pVac 21, pVac 22, pVac 23, pVac 24, pVac 25, pVac 26, pVac 27, pVac 28, pVac 29, pVac 30, pVac 31, pVac 32, pVac 33, pVac 34, pVac 35, pVac 36, pVac 37, pVac 38, pH1N1 Brisbane, pVac40, pVac42, pVac43, pVac44, pVac45, pVac46, pVac47, pVac48, pVac49, pVac50, pVac51, pVac52, pVac53, pVac54, pVac55, pVac56, pVac57, pVac58, pVac59, pVac60, pVac61, and pVac62.

61. The composition of any one of claims 1-60, wherein the composition is a pharmaceutical composition comprising a pharmaceutically acceptable carrier.

62. The composition of any one of claims 1-61, wherein the composition is a vaccine.

63. The composition, pharmaceutical composition, or vaccine of any one of claims 1-62, wherein the cationic polymer comprises a synthetic functionalized polymer, a β-amino ester, a lipid, a lipopolymer, or a chemical derivative thereof.

64. The composition, pharmaceutical composition, or vaccine of claim 63, wherein the synthetic functionalized polymer is a biodegradable cross-linked cationic multi-block copolymer.

65. The composition, pharmaceutical composition, or vaccine of claim 64, wherein the biodegradable cross-linked cationic multi-block copolymer is represented by the formula:
(CP)xLyYz, wherein:
(a) CP represents a cationic polymer containing at least one secondary amine group, wherein the cationic polymer has a number averaged molecular weight within the range of 1,000 to 25,000 Dalton,
(b) Y represents a bifunctional biodegradable linker containing ester, amide, disulfide, or phosphate linages,
(c) L represents a ligand,
(d) x is an integer in the range from 1 to 20,
(e) y is an integer in the range from 0 to 100, and
(f) z is an integer in the range from 0 to 40.

66. The composition, pharmaceutical composition, or vaccine of claim 65, wherein the cationic polymer comprises biodegradable cross-linked linear polyethyleneimine (LPEI).

67. The composition, pharmaceutical composition, or vaccine of claim 65 or 66, wherein the bifunctional biodegradable linker is hydrophilic and comprises a biodegradable linkage comprising a disulfide bond.

68. The composition, pharmaceutical composition, or vaccine of claim 65 or 66, wherein the bifunctional biodegradable linker is a dithiodipropionyl linker.

69. The composition, pharmaceutical composition, or vaccine of claim 64, wherein the biodegradable cross-linked cationic multi-block copolymer comprises LPEI and a dithiodipropionyl linker for cross-linking the multi-block copolymer, wherein the LPEI has an average molecular weight of 1,000 to 25,000 Dalton.

70. The composition, pharmaceutical composition, or vaccine of claim 69, wherein the biodegradable cross-linked cationic multi-block copolymer is covalently linked to at least one ligand.

71. The composition, pharmaceutical composition, or vaccine of claims 65-70, wherein the ligand is a targeting ligand selected from the group consisting of: a sugar moiety, a polypeptide, folate, and an antigen.

72. The composition, pharmaceutical composition, or vaccine of claim 71, wherein the sugar moiety is a monosaccharide or an oligosaccharide.

73. The composition, pharmaceutical composition, or vaccine of claim 72, wherein the monosaccharide is galactose.

74. The composition, pharmaceutical composition, or vaccine of claim 71, wherein the polypeptide is a glycoprotein, an antibody, an antibody fragment, a cell receptor, a cytokine receptor, or a growth factor receptor.

75. The composition, pharmaceutical composition, or vaccine of claim 74, wherein the growth factor receptor is an epidermal growth factor receptor.

76. The composition, pharmaceutical composition, or vaccine of claim 74, wherein the glycoprotein is transferrin or asialoorosomucoid (ASOR).

77. The composition, pharmaceutical composition, or vaccine of claim 71, wherein the antigen is a viral antigen, a bacterial antigen, or a parasite antigen.

78. The composition, pharmaceutical composition, or vaccine of any one of claims 64-77, wherein the biodegradable cross-linked cationic multi-block copolymer is covalently linked to polyethylene glycol (PEG) of molecular weight ranging from 500 to 20,000 Dalton.

79. The composition, pharmaceutical composition, or vaccine of any one of claims 59-73, wherein the biodegradable cross-linked cationic multi-block copolymer is covalently linked to a fatty acyl chain selected from the group consisting of: oleic acid, palmitic acid, and stearic acid.

80. The composition, pharmaceutical composition, or vaccine of any one of claims 64-79, wherein the biodegradable cross-linked cationic multi-block copolymer comprises at least one amine group that is electrostatically attracted to a polyanionic compound.

81. The composition, pharmaceutical composition, or vaccine of claim 80, wherein the polyanionic compound is a nucleic acid, wherein the biodegradable cross-linked cationic multi-block copolymer condenses the nucleic acid to form a compact structure.

82. The composition, pharmaceutical composition, or vaccine of claim 63, wherein the lipopolymer is a cationic lipopolymer comprising a PEI backbone covalently linked to a lipid or a PEG.

83. The composition, pharmaceutical composition, or vaccine of claim 82, wherein the PEI backbone is covalently linked to a lipid and a PEG.

84. The composition, pharmaceutical composition, or vaccine of claim 83, wherein the lipid and the PEG are directly attached to the PEI backbone by covalent bonds.

85. The composition, pharmaceutical composition, or vaccine of claim 83, wherein the lipid is attached to the PEI backbone through a PEG spacer.

86. The composition, pharmaceutical composition, or vaccine of any one of claims 82-85, wherein the PEG has a molecular weight of between 50 to 20,000 Dalton.

87. The composition, pharmaceutical composition, or vaccine of any one of claims 82-86, wherein the molar ratio of PEG to PEI is within a range of 0.1:1 to 500:1.

88. The composition, pharmaceutical composition, or vaccine of any one of claims 82-87, wherein the molar ratio of the lipid to the PEI is within a range of 0.1:1 to 500:1.

89. The composition, pharmaceutical composition, or vaccine of any one of claims 82-88, wherein the lipid is a cholesterol, a cholesterol derivative, a C12 to C18 fatty acid, or a fatty acid derivative.

90. The composition, pharmaceutical composition, or vaccine of claim 89, wherein the PEI is covalently linked to cholesterol and PEG, and wherein the average PEG:PEI:cholesterol molar ratio in the cationic lipopolymer is within the range of 1-5 PEG:1 PE:0.4-1.5 cholesterol.

91. The composition, pharmaceutical composition, or vaccine of any one of claims 82-90, wherein the PEI has a linear or branch configuration with a molecular weight of 100 to 500,000 Dalton.

92. The composition, pharmaceutical composition, or vaccine of any one of claims 82-91, wherein the cationic lipopolymer further comprises a pendant functional moiety selected from the group consisting of: a receptor ligand, a membrane permeating agent, an endosomolytic agent, a nuclear localization sequence, and a pH sensitive endosomolytic peptide.

93. The composition, pharmaceutical composition, or vaccine of any one of claims 82-92, wherein the cationic lipopolymer further comprises a targeting ligand, wherein the targeting ligand is directly attached to the PEI backbone or is attached through a PEG linker.

94. The composition, pharmaceutical composition, or vaccine of claim 93, wherein the targeting ligand is selected from the group consisting of: a sugar moiety, a polypeptide, folate, and an antigen.

95. The composition, pharmaceutical composition, or vaccine of claim 94, wherein the sugar moiety is a monosaccharide or an oligosaccharide.

96. The composition, pharmaceutical composition, or vaccine of claim 95, wherein the monosaccharide is galactose.

97. The composition, pharmaceutical composition, or vaccine of claim 94, wherein the polypeptide is a glycoprotein, an antibody, an antibody fragment, a cell receptor, a cytokine receptor, or a growth factor receptor.

98. The composition, pharmaceutical composition, or vaccine of any one of claims 1-97, wherein the cationic polymer is present in an amount sufficient to produce a ratio of amine nitrogen in the cationic polymer to phosphate in the DNA plasmid vector from about 0.01:1 to about 50:1.

99. The composition, pharmaceutical composition, or vaccine of claim 98, wherein the ratio of amine nitrogen in the cationic polymer to phosphate in the DNA plasmid vector from about 1:10 to about 10:1.

100. The composition, pharmaceutical composition, or vaccine of any one of claims 1-99, wherein the composition, comprises about 0.1 mg/ml to about 10.0 mg/ml nucleic acid complexed with the cationic polymer.

101. The composition, pharmaceutical composition, or vaccine of any one of claims 1-62, wherein the delivery component comprises a lipopolyamine with the following formula:

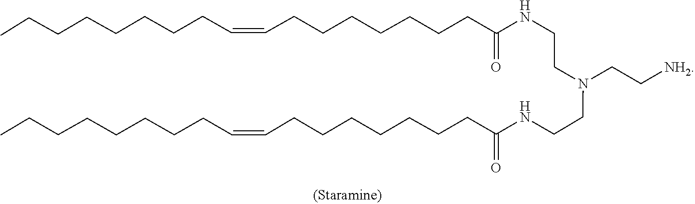 [SMILES: CCCCCCCC/C=C\CCCCCCCC(=O)NCCN(CCN)CCNC(=O)CCCCCCC/C=C\CCCCCCCC]

102. The composition, pharmaceutical composition, or vaccine of claim 101, wherein the delivery component comprises a mixture of the lipopolyamine and an alkylated derivative of the lipopolyamine.

103. The composition, pharmaceutical composition, or vaccine of claim 102, wherein the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride.

104. The composition, pharmaceutical composition, or vaccine of claim 103, wherein the alkylated derivative of the lipopolyamine has the following formula: 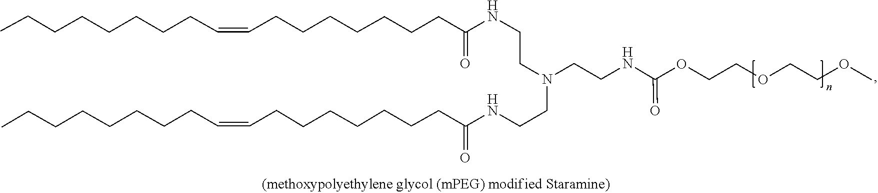
wherein n represents an integer from 10 to 100 repeating units containing of 2-5 carbon atoms each.

105. The composition, pharmaceutical composition, or vaccine of claim 102 or 103, wherein the ratio of the lipopolyamine to the alkylated derivative of the lipopolyamine in the mixture is 1:1 to 10:1.

106. The composition, pharmaceutical composition, or vaccine of any one of claims 103-105, wherein the lipopolyamine is present in an amount sufficient to produce a ratio of amine nitrogen in the lipopolyamine to phosphate in the DNA plasmid vector from about 0.01:1 to about 50:1.

107. The composition, pharmaceutical composition, or vaccine of claim 106, wherein the lipopolyamine is present in an amount sufficient to produce a ratio of amine nitrogen in the lipopolyamine to phosphate in the DNA plasmid vector from about 1:10 to about 10:1.

108. The composition, pharmaceutical composition, or vaccine of any one of claims 1-62, wherein the delivery component comprises a lipopolyamine with the following formula:

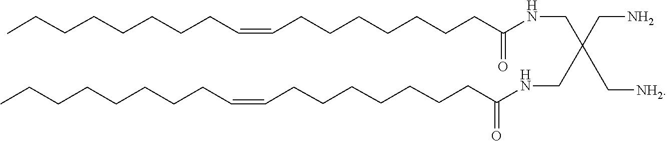 [SMILES: CCCCCCCC/C=C\CCCCCCCC(=O)NCC(CN)(CN)CNC(=O)CCCCCCC/C=C\CCCCCCCC]

109. The composition, pharmaceutical composition, or vaccine of claim 108, wherein the delivery component comprises a mixture of the lipopolyamine and an alkylated derivative of the lipopolyamine.

110. The composition, pharmaceutical composition, or vaccine of claim 109, wherein the alkylated derivative of the lipopolyamine is a polyoxyalkylene, polyvinylpyrrolidone, polyacrylamide, polydimethylacrylamide, polyvinyl alcohol, dextran, poly (L-glutamic acid), styrene maleic anhydride, poly-N-(2-hydroxypropyl) methacrylamide, or polydivinylether maleic anhydride.

111. The composition, pharmaceutical composition, or vaccine of claim 109 or 110, wherein the ratio of the lipopolyamine to the alkylated derivative of the lipopolyamine in the mixture is 1:1 to 10:1.

112. The composition, pharmaceutical composition, or vaccine of any one of claims 108-111, wherein the lipolyamine is present in an amount sufficient to produce a ratio of amine nitrogen in the lipopolyamine to phosphate in the DNA plasmid vector from about 0.01:1 to about 50:1.

113. The composition, pharmaceutical composition, or vaccine of claim 112, wherein the lipolyamine is present in an amount sufficient to produce a ratio of amine nitrogen in the lipopolyamine to phosphate in the DNA plasmid vector from about 0.1:10 to about 10:0.1.

114. The composition, pharmaceutical composition, or vaccine of any one of claims 1-62, wherein the delivery component comprises a poloxamer with the following formula: 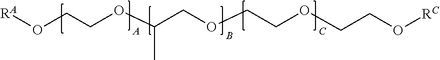
or a pharmaceutically acceptable salt thereof, wherein:
A represents an integer from 2 to 141;
B represents an integer from 16 to 67;
C represents an integer from 2 to 141;
RA and RC are the same or different, and are R′-L- or H;
L is a bond, —CO—, —CH₂—O—, or —O—CO—; and
R′ is a metal chelator.

115. The composition of claim 114, wherein at least one of RA and RC is R′-L-.

116. The composition, pharmaceutical composition, or vaccine of claim 114, wherein the R′ is covalently bound to the poloxamer.

117. The composition, pharmaceutical composition, or vaccine of claim 114-116, wherein one metal chelator or two or more metal chelators is/are bound to the poloxamer.

118. The composition, pharmaceutical composition, or vaccine of claim 114-117, wherein 2-100 metal chelators are bound to the poloxamer.

119. The composition of claim 114, wherein the metal chelator is RNNH—, RN2N—, or (R″—(N(R″)—CH2CH2)x)2-N—CH2CO—, wherein each x is independently 0-2, and wherein R″ is HO2C—CH2-.

120. The composition, pharmaceutical composition, or vaccine of claim 114, wherein the metal chelator is a crown ether, a substituted-crown ether, a cryptand, or a substituted-cryptand.

121. The composition, pharmaceutical composition, or vaccine of claim 114, wherein the delivery component further comprises a PEG-PEI-cholesterol lipopolymer, benzalkonium chloride, Omnifect, or a linear polyethyleneimine.

122. The composition, pharmaceutical composition, or vaccine of any one of claims 114-115, wherein the poloxamer is crown poloxamer and is present in a solution with the polynucleotide or DNA plasmid vector from about 0.01%-about 5%.

123. The composition, pharmaceutical composition, or vaccine of claim 122, wherein the solution is co-formulated with a metal chelator.

124. The composition, pharmaceutical composition, or vaccine of claim 123, wherein the co-formulated metal chelator is present in the solution at a concentration of about 0.1 mg/mL to about 20 mg/mL.

125. The composition, pharmaceutical composition, or vaccine of any one of claims 122-124, wherein the co-formulated metal chelator is crown ether, a substituted-crown ether, a cryptand, or a substituted-cryptand.

126. The composition, pharmaceutical composition, or vaccine of any one of claim 114-125, wherein the metal chelator and/or co-formulated metal chelator is crown ether (Aza-18-crown-6).

127. The composition, pharmaceutical composition, or vaccine of any one of claims 65-120, wherein the delivery component comprises BD15-12.

128. The composition, pharmaceutical composition, or vaccine of claim 127, wherein the nucleotide to polymer (N:P) ratio is 0.1:1 to 5:1.

129. The composition, pharmaceutical composition, or vaccine of any one of claims 65-120, wherein the delivery component is PEG-PEI-cholesterol lipopolymer.

130. The composition, pharmaceutical composition, or vaccine of claim 129, wherein the nucleotide to polymer (N:P) ratio is 0.1:1 to 5:1.

131. The composition, pharmaceutical composition, or vaccine of any one of claims 65-120, wherein the delivery component comprises Omnifect.

132. The composition, pharmaceutical composition, or vaccine of claim 131, wherein the nucleotide to polymer (N:P) ratio is 0.1:1 to 5:1.

133. The composition, pharmaceutical composition, or vaccine of any one of claims 65-120, wherein the delivery component comprises crown poloxamer connected by a covalent bond directly or through a linker to an aluminum or aluminum-salt based adjuvant.

134. The composition, pharmaceutical composition, or vaccine of claim 132, wherein the nucleotide to polymer (N:P) ratio is 0.1:1 to 5:1.

135. The composition, pharmaceutical composition, or vaccine of any one of claims 60-111, wherein the delivery component comprises Staramine and mPEG modified Staramine.

136. The composition, pharmaceutical composition, or vaccine of claim 130, wherein the mPEG modified Staramine is Staramine-mPEG515.

137. The composition, pharmaceutical composition, or vaccine of claim 135, wherein the mPEG modified Staramine is Staramine-mPEG11.

138. The composition, pharmaceutical composition, or vaccine of any one of claims 135-137, wherein the ratio of Staramine to mPEG modified Staramine is 10:1.

139. The composition, pharmaceutical composition, or vaccine of any one of claims 135-138, wherein the nucleotide to polymer (N:P) ratio is 0.01:1 to 5:1.

140. The composition, pharmaceutical composition, or vaccine of any one of claims 135-139, wherein the delivery component further comprises crown poloxamer.

141. The pharmaceutical composition, or vaccine of any one of claims 1-132, wherein the crown poloxamer is derivatized with a cationic molecule, a ligand, or other chemical entity.

142. The composition, pharmaceutical composition, or vaccine of any one of claims 1-140, wherein the composition is stable at 0° C. to 5° C. for at least about 1 month, about 2 months, about 3 months, about 4 months, about 5 months, about 6 months, about 7 months, about 8 months, about 9 months, about 10 months, about 11 months, about 12 months, about 24 months or about 36 months.

143. The composition, pharmaceutical composition, or vaccine of any one of claims 1-140, wherein the composition is stable at 25° C. for at least about 7 days, about 10 day, about 14 days, or about 60 days.

144. The composition, pharmaceutical composition, or vaccine of any one of claims 1-135, wherein the composition is stable at −20° C. for at least about 1 month, about 2 months, about 3 months, about 4 months, about 5 months, about 6 months, about 7 months, about 8 months, about 9 months, about 10 months, about 11 months, about 12 months, about 24 months or about 36 months.

145. The composition, pharmaceutical composition, or vaccine of any one of claims 1-141, wherein the composition is lyophilized and is substantially free of aqueous components.

146. The composition, pharmaceutical composition, or vaccine of claim 145, wherein the composition is reconstituted with a diluent.

147. The composition, pharmaceutical composition, or vaccine of claim 146, wherein the diluent is water.

148. A kit comprising the composition, pharmaceutical composition, or vaccine of any one of claims 1-147.

149. The kit of claim 148, further comprising a glass vial.

150. The kit of claim 148 or 149, further comprising instructions for using the composition or lyophilized composition in a method for inducing an immune response in a subject.

151. The kit of claim 149 or 150, further comprising instructions for using the composition or lyophilized composition in a method for preventing, reducing the incidence of, attenuating or treating an infection in a subject.

152. The kit of claim 151, wherein the infection is a viral infection, a bacterial infection, or a parasite infection.

153. The kit of claim 152, wherein the infection is a SARS-CoV-2 infection.

154. The kit of claim 152, wherein the infection is a Yersinia pestis infection, a Mycobacterium tuberculosis infection, a Meningococcus infection, an enterovirus infection, a herpes simplex virus (HSV) infection, a human immunodeficiency virus (HIV) infection, a human papillomavirus (HPV) infection, a hepatitis C virus (HCV) infection, a respiratory syncytial virus (RSV) infection, a Rabies virus infection, a Cytomegalovirus infection, a Yellow fever virus infection, a dengue virus infection, an Ebola virus infection, a Zika virus infection, a chikungunya virus infection, a measles virus infection, a Middle East Respiratory Syndrome Coronavirus (MERS-CoV) infection, a orthopoxvirus infection, a monkeypox virus infection, a vaccinia virus infection, a smallpox virus infection, a Epstein bar virus infection, a nipha virus infection, a varicella-zoster virus infection, a Clostridioides difficile infection, a Streptococcus pneumonia infection, a Neisseria meningitides infection, a Toxoplasma gondii infection, or a Plasmodium falciparum infection.

155. A method of inducing an immune response in a subject, the method comprising administering an effective amount of the composition, pharmaceutical composition, or vaccine of any one of claims 1-147 to the subject.

156. The method of claim 155, wherein the immune response is to one or more SARS-CoV-2 antigens.

157. The method of claim 150, wherein the immune response is to an antigen selected from a Yersinia pestis antigen, a Mycobacterium tuberculosis antigen, a Meningococcus antigen, an enterovirus antigen, a herpes simplex virus (HSV) antigen, a human immunodeficiency virus (HIV) antigen, a human papillomavirus (HPV) antigen, a hepatitis C virus (HCV) antigen, a respiratory syncytial virus (RSV) antigen, a Rabies virus antigen, a Cytomegalovirus antigen, a Yellow fever virus antigen, a dengue virus antigen, an Ebola virus antigen, a Zika virus, a chikungunya virus antigen, a measles virus antigen, a Middle East Respiratory Syndrome Coronavirus (MERS-CoV) antigen, a SARS-CoV antigen, a orthopoxvirus antigen, a monkeypox antigen, a vaccinia antigen, a smallpox antigen, a Epstein bar virus antigen, a nipha virus antigen, a varicella-zoster virus antigen, a Clostridioides difficile antigen, a Streptococcus pneumonia antigen, a Neisseria meningitides antigen, a Toxoplasma gondii antigen, a Plasmodium falciparum antigen, antigenic fragments thereof, and any combinations thereof.

158. The method of claim 157, wherein the immune response is to an antigen selected from a Yersinia pestis F1-Ag, a Yersinia pestis V-Ag, a Mycobacterium tuberculosis Apa antigen, a Mycobacterium tuberculosis HP65 antigen, a Mycobacterium tuberculosis rAg85A antigen, an E71 VP1 antigen, a GST-tagged E71-VP1 antigen, a Cox protein antigen, a GST-tagged Cox protein antigen, an HSV-1 envelope antigen, an HSV-2 envelope antigen, an HSV-2 gB2 antigen, an HSV-2 gC2 antigen, an HSV-2 gD2 antigen, an HSV-2 gE2 antigen, an HIV Env antigen, an HIV Gag antigen, an HIV Nef antigen, an HIV Pol antigen, an HPV minor capsid protein L2 antigen, a human papillomavirus type 16 Regulatory protein E2 antigen, a human papillomavirus type 16 Protein E6 antigen, a human papillomavirus type 16 Protein E7 antigen, a human papillomavirus type 18 Regulatory protein E2 antigen, a human papillomavirus type 18 Protein E6 antigen, a human papillomavirus type 18 Protein E7 antigen, a human papillomavirus type 6a Regulatory protein E2 antigen, a human papillomavirus type 6a Protein E6 antigen, a human papillomavirus type 6a Protein E7 antigen, a human papillomavirus 11 Regulatory protein E2 antigen, a human papillomavirus 11 Protein E6 antigen, a human papillomavirus 11 Protein E7 antigen, an HCV NS3 antigen, a hepatitis C virus genotype 1a Genome polyprotein antigen, a hepatitis C virus genotype 1b Genome polyprotein antigen, a hepatitis C virus genotype 2a Genome polyprotein antigen, a hepatitis C virus genotype 3a Genome polyprotein antigen, a RSV F antigen, a RSV G antigen, a Dengue virus E protein antigen, a Dengue virus EDIII antigen, a Dengue virus NS1 antigen, a Dengue virus DEN-80E antigen, an Ebola virus GB antigen, an Ebola virus VP24 antigen, an Ebola virus VP40 antigen, an Ebola virus NP antigen, an Ebola virus VP30 antigen, an Ebola virus VP35 antigen, a Zika virus envelope domain III antigen, a Zika virus CKD antigen, a Chikungunya virus E1 glycoprotein subunit antigen, the MHC class I epitope PPFGAGRPGQFGDI (SEQ ID NO: 34), the MHC class I epitope TAECKDKNL (SEQ ID NO: 35), the MHC class II epitope VRYKCNCGG (SEQ ID NO: 36), a measles virus hemagglutinin protein MV-H antigen, a measles virus fusion protein MV-F antigen, a MERS-CoV S protein antigen, an antigen from the receptor-binding domain of the MERS-CoV S protein, an antigen from the membrane fusion domain of the MERS-CoV S protein, a SARS-CoV S protein antigen, an antigen from the receptor binding domain of the SARS-CoV S protein, an antigen from the membrane fusion domain of the SARS-CoV S protein, a SARS-CoV E protein antigen, a SARS-CoV M protein antigen, a Delta variant SARS CoV-2 spike (S) protein, an Omicron variant SARS CoV-2 spike (S) protein, a Delta variant SARS-CoV-2 membrane (M) protein, an Omicron variant SARS-CoV-2 membrane (M) protein, a Delta variant SARS-CoV-2 envelope (E) protein, an Omicron variant SARS-CoV-2 envelope (E) protein, a Delta variant SARS-CoV-2 nucleocapsid (N) protein, an Omicron variant SARS-CoV-2 nucleocapsid (N) protein, a monkeypox A35R protein antigen, a monkeypox H₃L protein antigen, a monkeypox L1R protein antigen, a Clostridioides difficile 630 spore coat protein: peroxiredoxin/chitinase antigen, a Clostridioides difficile 630 flagellin C antigen, a Clostridioides difficile Surface layer protein A (Fragment) antigen, an Epstein-Barr virus (strain B95-8) nuclear antigen 1 antigen, an Epstein-Barr virus (strain B95-8) Envelope glycoprotein B antigen, an Epstein-Barr virus (strain B95-8) Envelope glycoprotein H antigen, an Epstein-Barr virus (strain B95-8) Envelope glycoprotein GP350 antigen, an Epstein-Barr virus (strain B95-8) Latent membrane protein 1 antigen, an Epstein-Barr virus (strain B95-8) Latent membrane protein 2 antigen, a Neisseria meningitides Factor H-binding protein antigen, a Neisseria meningitidis serogroup B Neisseria adhesin A antigen, a Neisseria meningitidis Neisserial heparin binding antigen antigen, a Vaccinia virus (strain Western Reserve) Protein A27 antigen, a Vaccinia virus (strain Western Reserve) EEV membrane phosphoglycoprotein antigen, a Vaccinia virus B5R (Fragment) antigen, a Vaccinia virus Envelope protein H₃ antigen, a Vaccinia virus (strain Western Reserve) IMV membrane protein antigen, a Nipah virus Fusion glycoprotein FO antigen, a Nipah virus Glycoprotein G antigen, a Varicella-zoster virus (strain Dumas) Envelope glycoprotein E antigen, a Toxoplasma gondii MIC8 antigen, a Plasmodium falciparum SERA5 polypeptide antigen, a Plasmodium falciparum circumsporozite protein antigen, antigenic fragments thereof, and any combination thereof.

159. The method of claim 155 or 158, wherein the immune response is a protective immune response.

160. A method of preventing, reducing the incidence of, attenuating or treating an infection in a subject, the method comprising administering an effective amount of the composition, pharmaceutical composition, or vaccine of any one of claims 1-147 to the subject.

161. The method of any one of claims 159-160, wherein the composition is administered to the subject by an intramuscular, subcutaneous, intralymphatic, transdermal, transnasal, or intraperitoneal route of administration.

162. The method of any one of claims 160-161, wherein the composition is administered to the subject between one and twenty times.

163. The method of any one of claims 160-162, wherein the composition is administered to the subject in an interval of from 1 day to about 14 weeks.

164. The method of ay one of claims 160-163, wherein the infection is a viral infection, a bacterial infection, or a parasite infection.

165. The method of any one of claims 160-164, wherein the infection is a SARS-CoV-2 infection.

166. The method of any one of claims 160-165, wherein the infection is a Yersinia pestis infection, a Mycobacterium tuberculosis infection, a Meningococcus infection, an enterovirus infection, a herpes simplex virus (HSV) infection, a human immunodeficiency virus (HIV) infection, a human papillomavirus (HPV) infection, a hepatitis C virus (HCV) infection, a respiratory syncytial virus (RSV) infection, a Rabies virus infection, a Cytomegalovirus infection, a Yellow fever virus infection, a dengue virus infection, an Ebola virus infection, a Zika virus infection, a chikungunya virus infection, a measles virus infection, a Middle East Respiratory Syndrome Coronavirus (MERS-CoV) infection, a orthopoxvirus antigen, a monkeypox antigen, a vaccinia antigen, a smallpox antigen, a Epstein bar virus antigen, a nipha virus antigen, a varicella-zoster virus antigen, a Clostridioides difficile antigen, a Streptococcus pneumonia antigen, a Neisseria meningitides antigen, a Toxoplasma gondii infection, or a Plasmodium falciparum infection.

167. A method of making a vaccine, the method comprising the steps of: (a) combining the delivery component with the polynucleotide of the composition or pharmaceutical composition of any one of claims 1-147, (b) lyophilizing the combined delivery component and polynucleotide to a powder, and (c) reconstituting the powder with a diluent that comprises the adjuvant to form a vaccine solution.
